(19) United States
(12) Patent Application Publication
(10) Pub. No.: US 2012/0101100 A1
(43) Pub. Date: Apr. 26, 2012

(54) HETEROCYCLIC UREA DERIVATIVES AND METHODS OF USE THEREOF-211

(75) Inventors: Shanta Bist, Waltham, MA (US); Brian Dangel, Waltham, MA (US); Brian Sherer, Waltham, MA (US)

(73) Assignee: ASTRAZENECA AB, Sodertalje (SE)

(21) Appl. No.: 12/392,730

(22) Filed: Feb. 25, 2009

Related U.S. Application Data
(60) Provisional application No. 61/031,621, filed on Feb. 26, 2008.

(51) Int. Cl. A61K 31/5377; A61K 31/444; A61P 31/04; A61K 31/5025; C07D 413/14; C07D 401/14; C07D 487/04

(52) U.S. Cl. 514/236.8; 546/256; 514/333; 544/236; 514/248; 544/131

(57) ABSTRACT

Compounds of formula (I) and their pharmaceutically acceptable salts are described. Processes for their preparation, pharmaceutical compositions containing them, their use as medicaments and their use in the treatment of bacterial infections are also described.

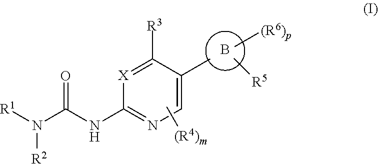

### RELATED APPLICATIONS

This application claims priority to U.S. 61/031,621 filed Feb. 26, 2008, the entire teachings of which are incorporated herein by reference.

### FIELD OF THE INVENTION

The present invention relates to compounds which demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. In particular, this invention relates to compounds useful for the treatment of bacterial infections in warm-blooded animals such as humans, more particularly to the use of these compounds in the manufacture of medicaments for use in the treatment of bacterial infections in warm-blooded animals such as humans.

### BACKGROUND OF THE INVENTION

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus aureus (MRSA), methicillin resistant coagulase negative staphylococci (MRCNS), penicillin resistant Streptococcus pneumoniae and multiple resistant Enterococcus faecium.

The preferred clinically effective antibiotic for treatment of last resort of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Consequently, in order to overcome the threat of widespread multi-drug resistant organisms, there is an on-going need to develop new antibiotics, particularly those with either a novel mechanism of action and/or containing new pharmacophoric groups.

Deoxyribonucleic acid (DNA) gyrase is a member of the type II family of topoisomerases that control the topological state of DNA in cells (Champoux, J. J.; 2001. Ann. Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

DNA gyrase is a well-validated target of antibacterials, including the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated primarily by mutations in the target (DNA gyrase and topoisomerase IV) is becoming an increasing problem in several organisms, including S. aureus and Streptococcus pneumoniae (Hooper, D. C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

Synthetic inhibitors that target the B subunit of DNA gyrase and topoisomeraselV are known in the art. For example, coumarin-containing compounds are described in patent application number WO 99/35155, 5,6-bicyclic heteroaromatic compounds are described in patent application WO 02/060879, and pyrazole compounds are described in patent application WO 01/52845 (U.S. Pat. No. 6,608,087). AstraZeneca has also published certain applications describing anti-bacterial compounds: WO2005/026149, WO2006/087544, WO2006/087548, WO2006/087543, WO2006/092599, WO2006/092608, WO2007/071965, WO2008/020227, WO2008/020222, WO2008/020229, WO2008/068470, and WO2008/152418.

### SUMMARY OF THE INVENTION

We have discovered a new class of compounds which are useful for inhibiting DNA gyrase and/or topoisomerase IV. The compounds of the present invention are regarded as effective against both Gram-positive and certain Gram-negative pathogens.

In one embodiment, according to the present invention there is provided a compound of formula (I):

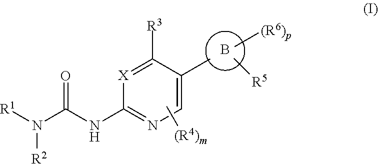

or a pharmaceutically acceptable salt thereof, wherein:
X is N, CH or CR⁴;
R¹ is selected from C₁₋₆alkyl, C₂₋₆alkenyl, C₂₋₆alkynyl or C₃₋₆cycloalkyl; wherein R¹ may be optionally substituted on carbon by one or more R⁷;
R² is selected from hydrogen or C₁₋₆alkyl; wherein said C₁₋₆alkyl may be optionally substituted by one or more groups independently selected from halo, cyano, hydroxy, nitro and amino;
or R¹ and R² together with the nitrogen to which they are attached form a heterocyclyl; wherein said heterocyclyl may be optionally substituted on one or more carbon atoms with one or more R⁸; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹;
R³ is a C₃₋₁₄carbocyclyl or a heterocyclyl; wherein the carbocyclyl or heterocyclyl may be optionally substituted on one or more carbon atoms by one or more R¹⁰; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹¹;
R⁴, for each occurrence, is independently selected from the group consisting of halo, nitro, cyano, hydroxy, amino, mercapto, C₁₋₆alkyl, C₂₋₆alkenyl, C₂₋₆alkynyl, C₁₋₆alkoxy, N—(C₁₋₆alkyl)amino, N,N—(C₁₋₆alkyl)₂amino, and C₁₋₆alkylsulfanyl; wherein R⁴, for each occurrence, is independently optionally substituted on one or more carbon atoms with one or more R¹²;
R⁵ is hydrogen or a heterocyclyl; wherein the heterocyclyl may be optionally substituted on one or more carbon atoms with an ═O, ═S, or one or more R¹⁴; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹⁵;
R⁶, for each occurrence, is independently selected from the group consisting of halo, nitro, cyano, hydroxy, amino, mercapto, sulphamoyl, ═O, ═S, C₁₋₆alkyl, C₂₋₆alkenyl, C₂₋₆alkynyl, C₁₋₆alkoxy, N—(C₁₋₆alkyl)amino, N,N—(C₁₋₆alkyl)₂amino, C₁₋₆ alkylS(O)ₐ— wherein a is 0, 1 or 2, N—(C₁₋₆alkyl)sulphamoyl, N,N—(C₁₋₆alkyl)₂sulphamoyl, C₁₋₆alkylsulphonylamino, N-hydroxycarbamimidoyl, carbamimidoyl, C₃₋₁₄carbocyclyl-L- and heterocyclyl-L-; wherein R⁶, for each occurrence, is independently optionally substituted on one or more carbon atoms with one or more R¹⁶; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹³;
m is 0 or 1;
p is 0, 1, 2, or 3;
Ring B is C₃₋₁₄carbocyclyl or heterocyclyl; wherein if said heterocyclyl contains an —NH— moiety that nitrogen may be optionally substituted by a group selected from R¹⁵; and wherein if said heterocyclyl contains an ═N— or a —S— moiety that nitrogen may be optionally substituted by one oxo group and that sulfur may be optionally substituted by one or two oxo groups;
R⁷, R⁸, R¹⁰, R¹², R¹⁴ and R¹⁶ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆carbocyclyl-L- or heterocyclyl-L-; wherein R⁷, R⁸, R¹⁰, R¹², R¹⁴ and R¹⁶ independently of each other may be optionally substituted on one or more carbon by one or more R¹⁹; and wherein if said heterocyclyl contains an —NH— moiety that nitrogen may be optionally substituted by a group selected from R²⁰; and wherein if said heterocyclyl contains an ═N— or a —S— moiety that nitrogen may be optionally substituted by one oxo group and that sulfur may be optionally substituted by one or two oxo groups
R⁹, R₁₁, R¹³, R¹⁵, and R²⁰, for each occurrence, are independently selected from C₁₋₆alkyl, C₃₋₆cycloalkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, imidazolylcarbonyl, amino, benzoyl and phenylsulphonyl; wherein R⁹, R¹¹, R¹³, R¹⁵, and R²⁰ independently of each other may be optionally substituted on carbon by one or more R²³;
R¹⁹ and R²³, for each occurrence, are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, 2-methoxyethoxy, morpholinyl, piperazinyl, acetyl, acetoxy, methylamino, ethylamino, dimethylamino, diethylamino, N-methyl-N-ethylamino, N-(2-morpholinoethyl)-amino, cyclohexylamino, cyclopentylamino, cyclohexyl, acetylamino, 2-methyoxyethylamino, tetrahydropyran-4-ylamino, N-methylcarbamoyl, N-ethylcarbamoyl, N,N-dimethylcarbamoyl, N,N-diethylcarbamoyl, N-methyl-N-ethylcarbamoyl, benzyloxy, 9H-fluoren-9-ylmethoxycarbonylamino, t-butoxycarbonylamino, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; and
L is a direct bond, —O—, —C(O)—, —C(O)NR²⁵—, —NR²⁵C(O)—, or —CH₂—; and
R²⁵ is H or a C₁₋₆alkyl.

In a particular embodiment, the present invention provides compounds having a structural formula (I) as recited above, or a pharmaceutically acceptable salt thereof, wherein:
R⁶, for each occurrence, is independently selected from the group consisting of halo, nitro, cyano, hydroxy, amino, mercapto, sulphamoyl, ═O, ═S, C₁₋₆alkyl, C₂₋₆alkenyl, C₂₋₆alkynyl, C₁₋₆alkoxy, N—(C₁₋₆alkyl)amino, N,N—(C₁₋₆alkyl)₂amino, C₁₋₆alkylS(O)ₐ— wherein a is 0, 1 or 2, N—(C₁₋₆alkyl)sulphamoyl, N,N—(C₁₋₆alkyl)₂sulphamoyl, C₁₋₆alkylsulphonylamino, C₃₋₁₄carbocyclyl and heterocyclyl; wherein R⁶, for each occurrence, is independently optionally substituted on one or more carbon atoms with one or more R¹⁶; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹³;
R⁷, R⁸, R¹⁰, R¹², R¹⁴ and R¹⁶ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆carbocyclyl or heterocyclyl; wherein R⁷, R⁸, R¹⁰, R¹², R¹⁴ and R¹⁶ independently of each other may be optionally substituted on one or more carbon by one or more R¹⁹; and wherein if said heterocyclyl contains an —NH— moiety that nitrogen may be optionally substituted by a group selected from R²⁰; and wherein if said heterocyclyl contains an ═N— or a —S— moiety that nitrogen may be optionally substituted by one oxo group and that sulfur may be optionally substituted by one or two oxo groups;
R⁹, R¹¹, R¹³, R¹⁵, and R²⁰, for each occurrence, are independently selected from C₁₋₆alkyl, C₃₋₆cycloalkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹¹, R¹³, R¹⁵, and R²⁰ independently of each other may be optionally substituted on carbon by one or more R²³; and
R¹⁹ and R²³, for each occurrence, are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl.

In another embodiment, the invention provides pharmaceutical compositions comprising a compound represented by formula (I), or a pharmaceutically acceptable salt thereof, and a pharmaceutically acceptable excipient or carrier.

In another embodiment, the invention provides a method of inhibiting bacterial DNA gyrase and/or bacterial topoisomerase IV in a warm-blooded animal in need of such treatment, comprising administering to the animal an effective amount of a compound represented by formula (I), or a pharmaceutically acceptable salt thereof. In a particular embodiment, the warm-blooded animal is a human.

In another embodiment, the invention provides a method of producing an antibacterial effect in a warm-blooded animal in need of such treatment, comprising administering to the animal an effective amount of a compound represented by formula (I), or a pharmaceutically acceptable salt thereof. In a particular embodiment, the warm-blooded animal is a human.

In another embodiment, the invention provides a method of treating a bacterial infection in a warm-blooded animal in need thereof, comprising administering to the animal an effective amount of a compound represented by formula (I), or a pharmaceutically acceptable salt thereof. In a particular embodiment, the warm-blooded animal is a human. In one embodiment, the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci. In a particular embodiment, the warm-blooded animal is a human.

In another embodiment, the invention provides the use of a compound represented by formula (I), or a pharmaceutically acceptable salt thereof, for the manufacture of a medicament for use in the production of an antibacterial effect in a warm-blooded animal. In a particular embodiment, the warm-blooded animal is a human.

In another embodiment, the invention provides the use of a compound represented by formula (I), or a pharmaceutically acceptable salt thereof, for the manufacture of a medicament for use in inhibition of bacterial DNA gyrase and/or topoisomerase IV in a warm-blooded animal. In a particular embodiment, the warm-blooded animal is a human.

In another embodiment, the invention provides the use of a compound represented by formula (I), or a pharmaceutically acceptable salt thereof, for the manufacture of a medicament for use the treatment of a bacterial infection in a warm-blooded animal. In one embodiment, the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis and Vancomycin-Resistant Enterococci. In a particular embodiment, the warm-blooded animal is a human.

In another embodiment, the invention provides a compound represented by formula (I), or a pharmaceutically acceptable salt thereof, for use in production of an anti-bacterial effect in a warm-blooded animal.

In another embodiment, the invention provides a compound represented by formula (I), or a pharmaceutically acceptable salt thereof, for use in inhibition of bacterial DNA gyrase and/or topoisomerase IV in a warm-blooded animal.

In another embodiment, the invention provides a compound represented by formula (I), or a pharmaceutically acceptable salt thereof, for use in the treatment of a bacterial infection in a warm-blooded animal.

In another embodiment, the invention provides a compound represented by formula (I), or a pharmaceutically acceptable salt thereof, for use in the treatment of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis or Vancomycin-Resistant Enterococci.

### DETAILED DESCRIPTION OF THE INVENTION

In this specification the term alkyl includes both straight chained and branched saturated hydrocarbon groups. For example, “C₁₋₆alkyl” refers to an alkyl that has from 1 to 6 carbon atom and includes, for example, methyl, ethyl, propyl, isopropyl and t-butyl. However references to individual alkyl groups such as propyl are specific for the straight chain version only unless otherwise indicated (e.g., isopropyl). An analogous convention applies to other generic terms.

As used herein, the term “C₁₋₆haloalkyl” refers to an alkyl group that has from 1 to 6 carbon atoms in which one or more of the carbon atoms are substituted with a halo group. Representative haloalkyl groups include —CF₃, —CHF₂, —CCl₃, —CH₂CH₂Br, —CH₂CH(CH₂CH₂Br)CH₃, —CHICH₃, and the like.

As used herein, the term “halo” refers to fluoro, chloro, bromo, and iodo.

A “heterocyclyl” is a saturated, partially saturated or unsaturated, mono or bicyclic ring containing 4-14 atoms of which at least one atom is chosen from nitrogen, sulphur or oxygen, which may, unless otherwise specified, be carbon or nitrogen linked, wherein a —CH₂— group can optionally be replaced by a —C(O)— and a ring sulphur atom may be optionally oxidised to form the S-oxide(s). In one embodiment of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides. In a further aspect of the invention a “heterocyclyl” is an unsaturated, carbon-linked, monocyclic ring containing 5 or 6 atoms of which at least one atom is chosen from nitrogen, sulphur or oxygen. Examples and suitable values of the term “heterocyclyl” are morpholinyl, piperidyl, pyridinyl, pyranyl, pyrrolyl, pyrazolyl, isothiazolyl, indolyl, quinolinyl, thienyl, 1,3-benzodioxolyl, benzothiazolyl, thiadiazolyl, oxadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, 4,5-dihydro-oxazolyl, pyrimidinyl, pyrazinyl, pyridazinyl, isoxazolyl, thiazolyl, 1H-tetrazolyl, 1H-triazolyl, N-methylpyrrolyl, 4-pyridone, quinolin-4(1H)-one, pyridin-2(1H)-one, imidazo[1,2-a]pyridinyl, 1-isoquinolone, 2-pyrrolidone, 4-thiazolidone, quinoxalinyl, 5,6-dihydro[1,3]thiazolo[4,5-d]pyridazinyl, pyridine-N-oxide and quinoline-N-oxide. Suitable examples of “a nitrogen linked heterocyclyl” are morpholino, piperazin-1-yl, piperidin-1-yl and imidazol-1-yl. The term “heterocyclyl” encompasses the term “heteroaryl.” A “heteroaryl” is an aromatic mono-, bi- or tricyclic heterocycle.

A “carbocyclyl” is a saturated, partially saturated or unsaturated, mono-, bi- or tricyclic carbon ring that contains 3-14 atoms; wherein a —CH₂— group can optionally be replaced by a —C(O)—. In one embodiment, “carbocyclyl” is a monocyclic ring containing 5 or 6 atoms or a bicyclic ring containing 9 or 10 atoms. Examples of carbocyclyls include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. The term carbocyclyl encompasses both cycloalkyl and aryl groups. The term cycloalkyl refers to a carbocyclyl which is completely saturated, for example cyclopropyl, cyclobutyl, cyclopentyl, and cyclohexyl. The term “aryl” refers to a carbocyclyl which is completely unsaturated and is aromatic. A C₆₋₁₄aryl is an aromatic, mono-, bi- or tricyclic carbon ring that contains 6-14 atoms, for example phenyl or naphthenyl.

An example of “C₁₋₆alkanoyloxy” is acetoxy. Examples of “C₁₋₆alkoxycarbonyl” are methoxycarbonyl, ethoxycarbonyl, n- and t-butoxycarbonyl. Examples of “C₁₋₆alkoxycarbonylamino” are methoxycarbonylamino, ethoxycarbonylamino, n- and t-butoxycarbonylamino Examples of “C₁₋₆alkoxy” are methoxy, ethoxy and propoxy. Examples of “C₁₋₆alkanoylamino” are formamido, acetamido and propionylamino Examples of “C₁₋₆alkylS(O)ₐ wherein a is 0, 1, or 2” are methylthio, ethylthio, methylsulphinyl, ethylsulphinyl, mesyl and ethylsulphonyl. Examples of “C₁₋₆alkanoyl” are propionyl and acetyl. Examples of “N—(C₁₋₆alkyl)amino” are methylamino and ethylamino Examples of “N,N—(C₁₋₆alkyl)₂amino” are di-N-methylamino, di-(N-ethyl)amino and N-ethyl-N-methylamino Examples of “C₂₋₄alkenyl” are vinyl, allyl and 1-propenyl. Examples of “C₂₋₄alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N,N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are methylaminocarbonyl and ethylaminocarbonyl. Examples of “N,N—(C₁₋₆alkyl)₂carbamoyl” are dimethylaminocarbonyl and methylethylaminocarbonyl. Examples of “N—(C₁₋₆alkoxy)carbamoyl” are methoxyaminocarbonyl and isopropoxyaminocarbonyl. Examples of “N—(C₁₋₆alkyl)-N—(C₁₋₆alkoxy)carbamoyl” are N-methyl-N-methoxyaminocarbonyl and N-methyl-N-ethoxyaminocarbonyl. Examples of “C₃₋₆cycloalkyl” are cyclopropyl, cyclobutyl, cyclopropyl and cyclohexyl. Examples of “C₁₋₆alkylsulphonylamino” are methylsulphonylamino, isopropylsulphonylamino and t-butylsulphonylamino Examples of “C₁₋₆alkylsulphonylaminocarbonyl” are methylsulphonylaminocarbonyl, isopropylsulphonylaminocarbonyl and t-butylsulphonylaminocarbonyl. Examples of “C₁₋₆alkylsulphonyl” are methylsulphonyl, isopropylsulphonyl and t-butylsulphonyl.

A compound of formula (I) may form stable acid or basic salts, and in such cases administration of a compound as a salt may be appropriate, and pharmaceutically acceptable salts may be made by conventional methods such as those described below.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

Within the present invention it is to be understood that a compound of the formula (I), or a salt thereof may exhibit the phenomenon of tautomerism and that the formulae drawings within this specification can represent only one of the possible tautomeric forms. It is to be understood that the invention encompasses any tautomeric form which inhibits DNA gyrase and/or topoisomerase IV and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names.

It will be appreciated by those skilled in the art that certain compounds of formula (I) contain an asymmetrically substituted carbon and/or sulphur atom, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which form possesses properties useful in the inhibition of DNA gyrase and/or topoisomerase IV, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DNA gyrase and/or topoisomerase IV by the standard tests described hereinafter.

By way of clarity, compounds of the invention included all isotopes of the atoms present in formula (I) and any of the examples or embodiments disclosed herein. For example, H (or hydrogen) represents any isotopic form of hydrogen including ¹H, ²H (D), and ³H (T); C represents any isotopic form of carbon including ¹²C, ¹³C, and ¹⁴C; O represents any isotopic form of oxygen including ¹⁶O, ¹⁷O and ¹⁸O; N represents any isotopic form of nitrogen including ¹³N, ¹⁴N and ¹⁵N; P represents any isotopic form of phosphorous including ³¹P and ³²P; S represents any isotopic form of sulfur including ³²S and ³⁵S; F represents any isotopic form of fluorine including ¹⁹F and ¹⁸F; Cl represents any isotopic form of chlorine including ³⁵Cl, ³⁷Cl and ³⁶Cl; and the like. In a preferred embodiment, compounds represented by formula (I) comprises isomers of the atoms therein in their naturally occurring abundance. However, in certain instances, it is desirable to enrich one or more atom in a particular isotope which would normally be present in less abundance. For example, ¹H would normally be present in greater than 99.98% abundance; however, a compound of the invention can be enriched in ²H or ³H at one or more positions where H is present. In particular embodiments of the compounds of formula (I), when, for example, hydrogen is enriched in the deuterium isotope, the symbol “D” may be used to represent the enrichment in deuterium. In one embodiment, when a compound of the invention is enriched in a radioactive isotope, for example ³H and ¹⁴C, they may be useful in drug and/or substrate tissue distribution assays. It is to be understood that the invention encompasses all such isotopic forms which inhibit DNA gyrase and/or topoisomerase IV.

It is also to be understood that certain compounds of the formula (I), and salts thereof can exist in solvated as well as unsolvated forms such as, for example, hydrated forms. It is to be understood that the invention encompasses all such solvated forms which inhibit DNA gyrase and/or topoisomerase IV.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

In one embodiment the invention provides compounds represented by formula (I) wherein X is CH.

In another embodiment the invention provides compounds represented by formula (I) wherein X is N.

In another embodiment the invention provides compounds represented by formula (I) wherein X is CR⁴ and R⁴ is fluoro, chloro, bromo, iodo, a C₁₋₄alkyl, or a C₁₋₄alkoxy.

In another embodiment the invention provides compounds represented by formula (I) wherein ring B is a 5- or 6-membered heteroaryl, and wherein if said heteroaryl contains an —NH— moiety that nitrogen may be optionally substituted by a group selected from R¹⁵; and wherein if said heteroaryl contains an ═N— or a —S— moiety that nitrogen may be optionally substituted by one oxo group and that sulfur may be optionally substituted by one or two oxo groups.

In another embodiment the invention provides compounds represented by formula (I) wherein ring B is pyridinyl, pyrazinyl, pyrimidinyl or thiazolyl; and wherein each ═N— of pyridinyl, pyrazinyl, pyrimidinyl, or thiazolyl may be independently optionally substituted with one oxo group; and wherein the —S— moiety of the thiazolyl may be optionally by one or two oxo groups.

In another embodiment the invention provides compounds represented by formula (I) wherein ring B is pyridinyl, pyrazinyl, pyridazinyl, pyrimidinyl or thiazolyl; and wherein each ═N— of pyridinyl, pyrazinyl, pyrimidinyl, or thiazolyl may be independently optionally substituted with one oxo group; and wherein the —S— moiety of the thiazolyl may be optionally by one or two oxo groups.

In another embodiment the invention provides compounds represented by formula (I) wherein ring B is a bicyclic heterocyclyl; and wherein if said heterocyclyl contains an —NH— moiety that nitrogen may be optionally substituted by a group selected from R¹⁵; and wherein if said heterocyclyl contains an ═N— or a —S— moiety that nitrogen may be optionally substituted by one oxo group and that sulfur may be optionally substituted by one or two oxo groups.

In another embodiment the invention provides compounds represented by formula (I) wherein ring B is a quinoxalinyl or 5,6-dihydro[1,3]thiazolo[4,5-d]pyridazine-4,7-dione; and wherein each —NH— moiety of 5,6-dihydro[1,3]thiazolo[4,5-d]pyridazine-4,7-dione may be independently optionally substituted by a group selected from R¹⁵; and wherein each ═N— of quinoxalinyl or 5,6-dihydro[1,3]thiazolo[4,5-d]pyridazine-4,7-dione may be independently optionally substituted with one oxo group; and wherein the —S— moiety of the 5,6-dihydro[1,3]thiazolo[4,5-d]pyridazine-4,7-dione may be optionally by one or two oxo groups.

In another embodiment the invention provides compounds represented by formula (I) wherein ring B is a quinoxalinyl, 5,6-dihydro[1,3]thiazolo[4,5-d]pyridazine-4,7-dione or 2,3-dihydrophthalazine-1,4-dione; and wherein each —NH— moiety of 5,6-dihydro[1,3]thiazolo[4,5-d]pyridazine-4,7-dione or 2,3-dihydrophthalazine-1,4-dione may be independently optionally substituted by a group selected from R¹⁵; and wherein each ═N— of quinoxalinyl or 5,6-dihydro[1,3]thiazolo[4,5-d]pyridazine-4,7-dione may be independently optionally substituted with one oxo group; and wherein the —S— moiety of the 5,6-dihydro[1,3]thiazolo[4,5-d]pyridazine-4,7-dione may be optionally by one or two oxo groups.

In another embodiment the invention provides compounds represented by formula (I) wherein R¹ is a C₁₋₆alkyl which is optionally substituted by a halo. For example, R¹ is methyl, ethyl, propyl, isopropyl, butyl, isobutyl, sec-butyl, and tert-butyl, 2,2,2-trifluoroethyl, or 2,2-difluoroethyl. In a particular embodiment, R¹ is ethyl.

In another embodiment the invention provides compounds represented by formula (I) wherein R¹ is a C₁₋₆alkyl. For example, R¹ is methyl, ethyl, propyl, isopropyl, butyl, isobutyl, sec-butyl, and tert-butyl. In a particular embodiment, R¹ is ethyl.

In another embodiment the invention provides compounds represented by formula (I) wherein R¹ is a C₁₋₆alkyl which is substituted with a halo. For example, R¹ is 2,2,2-trifluoroethyl or 2,2-difluoroethyl.

In another embodiment the invention provides compounds represented by formula (I) wherein R¹ is a C₃₋₆cylcoalkyl. For example, R¹ is cyclopropyl or cyclohexyl.

In another embodiment the invention provides compounds represented by formula (I) wherein R² is hydrogen.

In another embodiment the invention provides compounds represented by formula (I) wherein R² is a C₁₋₆alkyl. For example, R² is methyl, ethyl, propyl, isopropyl, butyl, isobutyl, sec-butyl, and tert-butyl.

In another embodiment the invention provides compounds represented by formula (I) wherein R³ is a 5-membered heteroaryl; and wherein the heteroaryl may be optionally substituted on one or more carbon atoms by one or more R¹⁰; and wherein if said heteroaryl contains an ═N— or a —S— moiety that nitrogen may be optionally substituted by one oxo group and that sulfur may be optionally substituted by one or two oxo groups; and wherein if said heteroaryl contains an —NH— moiety that nitrogen may be optionally substituted by a group selected from R¹¹. In one aspect of this embodiment, R¹⁰, for each occurrence, is selected from the group consisting of methyl, phenyl, trifluoromethyl, and pyridinyl. In another aspect of this embodiment, R¹¹ is methyl.

In another embodiment the invention provides compounds represented by formula (I) wherein R³ is a thiazolyl; and wherein the thiazolyl may be optionally substituted on carbon by one or more R¹⁰; and wherein the ═N— of the thiazolyl may be optionally substituted by one oxo group; and wherein the —S— of the thiazolyl may be optionally substituted by one or two oxo groups. In one aspect of this embodiment, R¹⁰, for each occurrence, is independently selected from the group consisting of methyl, phenyl, trifluoromethyl, and pyridinyl. In one aspect of this embodiment, R¹⁰, for each occurrence, is independently selected from the group consisting of methyl, phenyl, trifluoromethyl, pyridinyl, 1-methyl-1H-pyrazol-4-yl, N-(2-morpholinoethyl)aminomethyl, N-cyclohexylaminomethyl, cyclopentylaminomethyl, N-(2-methoxyethyl)aminomethyl, N-(tetrahydro-2H-pyran-4-yl)aminomethyl, N-(2-methoxyethyl)-carbamoyl, N-(2-morpholinoethyl)-carbamoyl, N-[2-(N-methyl-piperazino)-ethyl]-carbamoyl, N-cyclopropyl-carbamoyl, N-cyclopentyl-carbamoyl, N-cyclohexyl-carbamoyl, methoxy, 6-methoxypyridin-2-yl, 6-methoxypyridin-3-yl, 2-fluoropyridin-3-yl, 2-(2-methoxyethoxy)pyridin-2-yl, 6-methoxypyridin-2-yl, pyridin-4-ylmethyl, cyclopropyl, 2,2-dimethyl-2H-tetrahydropyran-4-yl, N-(1H-imidazol-1-ylcarbonyl)-piperidin-4-yl, cyclopentyl, and cyclohexyl. In another aspect R¹⁰ is trifluoromethyl.

In another embodiment the invention provides compounds represented by formula (I) wherein R³ is a 1,3,4-oxadiazolyl; and wherein the 1,3,4-oxadiazolyl may be optionally substituted on one or more carbon by one or more R¹⁰; and wherein each ═N— of the 1,3,4-oxadiazolyl may be independently optionally substituted by one oxo group. In one aspect of this embodiment, R¹⁰, for each occurrence, is independently selected from the group consisting of methyl, phenyl, trifluoromethyl, and pyridinyl. In another aspect of this embodiment, R¹⁰, for each occurrence, is selected from pyridinyl, phenyl, and 4-fluorophenyl.

In another embodiment the invention provides compounds represented by formula (I) wherein R³ is a 1H-pyrazolyl; and wherein the 1H-pyrazolyl may be optionally substituted on one or more carbon by one or more R¹⁰; and wherein the ═N— of the 1H-pyrazolyl may be optionally substituted by one oxo group; and wherein the —NH— moiety of the 1H-pyrazolyl may be optionally substituted by a group selected from R¹¹. In one aspect of this embodiment, R¹⁰, for each occurrence, is independently selected from the group consisting of methyl, phenyl, trifluoromethyl, and pyridinyl. In another aspect of this embodiment, R¹¹ is methyl. In another aspect of this embodiment, R¹¹ is methyl, 2-morpholinoethyl, or isopropyl.

In another embodiment the invention provides compounds represented by formula (I) wherein R³ is a 1H-1,2,3-triazolyl; and wherein the 1H-1,2,3-triazolyl may be optionally substituted on one or more carbon by one or more R¹⁰; and wherein the ═N— of the 1H-1,2,3-triazolyl may be optionally substituted by one oxo group; and wherein the —NH— moiety of the 1H-1,2,3-triazolyl may be optionally substituted by a group selected from R¹¹. In one aspect of this embodiment, R¹⁰, for each occurrence, is independently selected from the group consisting of methyl, phenyl, trifluoromethyl, and pyridinyl. In another aspect of this embodiment, R¹¹ is benzyl.

In another embodiment the invention provides compounds represented by formula (I) wherein R³ is 1,3-benzothiazolyl; and wherein the 1,3-benzothiazolyl may be optionally substituted on one or more carbon by one or more R¹⁰; and wherein the ═N— of the 1,3-benzothiazolyl may be optionally substituted by one oxo group; and wherein the —S— of the 1,3-benzothiazolyl may be optionally substituted by one or two oxo groups. In one aspect of this embodiment, R¹⁰ is selected from the group consisting of methyl, phenyl, trifluoromethyl, and pyridinyl.

In another embodiment the invention provides compounds represented by formula (I) wherein R³ is 4-trifluoromethyl-thiazol-2-yl, 4-(pyridin-2-yl)-thiazol-2-yl, 4-phenyl-thiazol-2-yl, 1,3-benzothiazol-2-yl, 2-(pyridin-4-yl)-1,3,4-oxadiazol-5-yl, 1-methyl-1H-pyrazol-5-yl, 1-methyl-1H-pyrazol-4-yl, 2-methyl-1,3,4-oxadiazol-5-yl, or 4-(pyridin-4-yl)-thiazol-2-yl.

In another embodiment the invention provides compounds represented by formula (I) wherein R³ is an aryl which may be optionally substituted on one or more carbon atoms with one or more R¹⁰.

In another embodiment the invention provides compounds represented by formula (I) wherein R³ is a morpholinyl wherein the morpholinyl may be optionally substituted on one or more carbon atoms with one or more R¹⁰, and wherein the —NH— moiety of the morpholinyl may be optionally substituted by a group selected from R¹¹.

In another embodiment the invention provides compounds represented by formula (I) wherein R³ is a piperidinyl wherein the piperidinyl may be optionally substituted on one or more carbon atoms with one or more R¹⁰, and wherein the —NH— moiety of the piperidinyl may be optionally substituted by a group selected from R¹¹.

In one embodiment, R⁵ is hydrogen.

In another embodiment the invention provides compounds represented by formula (I) wherein R⁵ is a five membered aromatic heterocyclyl; wherein the heterocyclyl may be optionally substituted on one or more carbon atoms with one or more R¹⁴; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹⁵. In one aspect of this embodiment, R¹⁴, for each occurrence, is independently selected from the group consisting of C₁₋₄alkyl and hydroxy. In another aspect of this embodiment, R¹⁵ is a C₁₋₄alkyl.

In one embodiment, R⁵ is a 5-oxo-4,5-dihydro-1,3,4-oxadiazolyl-2-yl wherein the 5-oxo-4,5-dihydro-1,3,4-oxadiazolyl-2-yl may be optionally substituted on one or more carbon atoms with one or more R¹⁴; and wherein the ═N— moiety of the 5-oxo-4,5-dihydro-1,3,4-oxadiazolyl-2-yl may be optionally substituted by one oxo group and wherein the —NH— moiety of the 5-oxo-4,5-dihydro-1,3,4-oxadiazolyl-2-yl may be optionally substituted by a group selected from R¹⁵. In a particular embodiment, R⁵ is a 5-oxo-4,5-dihydro-1,3,4-oxadiazolyl-2-yl.

In one embodiment, R⁵ is a 1,3,4-oxadiazolyl wherein the 1,3,4-oxadiazolyl may be optionally substituted on one or more carbon atoms with one or more R¹⁴; and wherein the ═N— moieties of the 1,3,4-oxadiazolyl may be independently optionally substituted by one oxo group. In a particular embodiment, R⁵ and R¹⁴ together are a 5-methyl-1,3,4-oxadiazol-2-yl. In another particular embodiment, R⁵ and R¹⁴ together are selected from 5-isopropyl-1,3,4-oxadiazol-2-yl, 5-amino-1,3,4-oxadiazol-2-yl, a 5-(1-amino-isobutyl)-1,3,4-oxadiazol-2-yl, 5-[3-(N,N-dimethylamino)-propylamino]-1,3,4-oxadiazol-2-yl, 5-morpholino-1,3,4-oxadiazol-2-yl, 5-(morpholin-3-yl)-1,3,4-oxadiazol-2-yl, 5-cyclopropyl-1,3,4-oxadiazol-2-yl, 5-(3-hydroxypiperidino)-1,3,4-oxadiazol-2-yl, 5-(4-hydroxypiperidino)-1,3,4-oxadiazol-2-yl, 5-(3-hydroxyazetidino)-1,3,4-oxadiazol-2-yl, 5-(1-hydroxyethyl)-1,3,4-oxadiazol-2-yl, 5-(1-hydroxyisopropyl)-1,3,4-oxadiazol-2-yl, 5-(1-acetoxyisopropyl)-1,3,4-oxadiazol-2-yl, 542-oxo-propyl)-1,3,4-oxadiazol-2-yl, 5-benzyloxymethyl-1,3,4-oxadiazol-2-yl, 5-(N,N-diethylamino)-1,3,4-oxadiazol-2-yl, 5-(N,N-dimethylaminomethyl)-1,3,4-oxadiazol-2-yl, 5-(methoxymethyl)-1,3,4-oxadiazol-2-yl, 5-ethoxy-1,3,4-oxadiazol-2-yl, 1,3,4-oxadiazol-2-yl, 5-(1-hydroxycyclopropyl)-1,3,4-oxadiazol-2-yl, 5-(N,N-dimethylcarbamoyl)-1,3,4-oxadiazol-2-yl, 5-(2-methoxyethoxymethyl)-1,3,4-oxadiazol-2-yl, 5-(1-amino-1-cyclohexylmethyl)-1,3,4-oxadiazol-2-yl, and 5-(aminomethyl)-1,3,4-oxadiazol-2-yl.

In another embodiment the invention provides compounds represented by formula (I) wherein R⁵ is selected from the group consisting of 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, morpholinyl, 4,5-dihydro-oxazolyl, and 1H-1,2,4-triazolyl, wherein the 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, morpholinyl, 4,5-dihydro-oxazolyl, and 1H-1,2,4-triazolyl may be optionally substituted on one or more carbon atoms with one or more R¹⁴; and wherein the ═N— moiety of 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, 4,5-dihydro-oxazolyl, and 1H-1,2,4-triazolyl may be optionally substituted with one oxo group and the —S— moiety of 1,3,4-thiadiazolyl or 3H-1,2,3,5-oxathiadiazolyl may be optionally substituted by one or two oxo groups; and wherein the —NH— moiety of the 1H-tetrazolyl, 1H-pyrazolyl, 3H-1,2,3,5-oxathiadiazolyl, 1H-imidazolyl, morpholinyl, or the 1H-1,2,4-triazolyl may be optionally substituted by a group selected from R¹⁵. In one aspect of this embodiment, R¹⁴ is selected from the group consisting of C₁₋₄alkyl or hydroxy. In another aspect of this embodiment, R¹⁵ is a C₁₋₄alkyl.

In another embodiment the invention provides compounds represented by formula (I) wherein R⁵ is selected from the group consisting of 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, morpholinyl, 4,5-dihydro-oxazolyl, oxazolyl, thiazolyl, and 1H-1,2,4-triazolyl, wherein the 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, morpholinyl, 4,5-dihydro-oxazolyl, and 1H-1,2,4-triazolyl may be optionally substituted on one or more carbon atoms with one or more R¹⁴; and wherein the ═N— moiety of 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, 4,5-dihydro-oxazolyl, and 1H-1,2,4-triazolyl may be optionally substituted with one oxo group and the —S— moiety of 1,3,4-thiadiazolyl or 3H-1,2,3,5-oxathiadiazolyl may be optionally substituted by one or two oxo groups; and wherein the —NH— moiety of the 1H-tetrazolyl, 1H-pyrazolyl, 3H-1,2,3,5-oxathiadiazolyl, 1H-imidazolyl, morpholinyl, or the 1H-1,2,4-triazolyl may be optionally substituted by a group selected from R¹⁵. In one aspect of this embodiment, R¹⁴ is selected from the group consisting of C₁₋₄alkyl or hydroxy. In another aspect of this embodiment, R¹⁵ is a C₁₋₄alkyl.

In another embodiment the invention provides compounds represented by formula (I) wherein R¹⁴ is selected from methyl, isopropyl, amino, trifluoromethyl, difluoromethyl, 1-amino-isobutyl, 3-(N,N-dimethylamino)-propylamino, morpholino, morpholin-3-yl, cyclopropyl, 3-hydroxypiperidino, 4-hydroxypiperidino, 3-hydroxyazetidino, 1-hydroxyethyl, 1-hydroxyisopropyl, 1-acetoxyisopropyl, 2-oxo-propyl, benzyloxymethyl, N,N-diethylamino, N,N-dimethylaminomethyl, methoxymethyl, ethoxy, 1-hydroxycyclopropyl, N,N-dimethylcarbamoyl, 2-methoxyethoxymethyl, 1-amino-1-cyclohexylmethyl, and aminomethyl).

In another embodiment the invention provides compounds represented by formula (I) wherein R¹⁵ is selected from methyl, morpholinocarbonyl, and piperidinocarbonyl.

In another embodiment the invention provides compounds represented by formula (I) wherein m is 0.

In another embodiment the invention provides compounds represented by formula (I) wherein m is 0 and X is CH.

In another embodiment the invention provides compounds represented by formula (I) wherein m is 0 and X is N.

In another embodiment the invention provides compounds represented by formula (I) wherein p is 0.

In another embodiment the invention provides compounds represented by formula (I) wherein p is 0 and R⁵ is hydrogen. In one aspect of this embodiment, ring B is pyridine or quinoxalinyl.

In another embodiment the invention provides compounds represented by formula (I) wherein p is 1. In one aspect of this embodiment, R⁶ is cyano, bromo, methylsulfonyl, sulphamoyl, or butyloxy.

In another embodiment the invention provides compounds represented by formula (I) wherein p is 1 and R⁵ is hydrogen. In one aspect of this embodiment, R⁶ is cyano, bromo, methylsulfonyl, sulphamoyl, or butyloxy.

In another embodiment the invention provides compounds represented by formula (I) wherein p is 2. In one aspect of this embodiment, R⁶, for each occurrence, is independently selected from cyano, bromo, methylsulfonyl, sulphamoyl, and butyloxy.

In another embodiment the invention provides compounds represented by formula (I) wherein p is 3. In one aspect of this embodiment, R⁶, for each occurrence, is independently selected from cyano, bromo, methylsulfonyl, sulphamoyl, and butyloxy.

In another embodiment the invention provides compounds represented by formula (I) wherein R⁶, for each occurrence, is independently selected cyano, fluoro, bromo, ethyl, methylsulfonyl, sulphamoyl, methylsulfonyl, N′ hydroxycarbamimidoyl, carbamimidoyl, pyrrolidinoethoxy, butyloxy, methoxy, ethoxy, isopropoxy, morpholino, cyclopropylmethoxy, N-methylpiperidin-4-yloxy, N-methyl-1H-1,2,4-triazol-5-yl, 5-methyl-1,3,4-oxadiazol-2-yl, pyrimidin-2-yl, N-methyl-piperazin-1-ylethoxy, N-methyl-piperazin-1-ylmethoxy, 2-(N,N-dimethylamino)-ethoxy, 2-morpholinoethoxy, piperidin-4-yloxy, 2-carboxyethoxy, 2H-tetrahydropyran-4-ylmethoxy, 1-methyl-2-(N,N-dimethylamino)-ethoxy, 2-(N,N-diethylamino)-ethoxy, 2-(N,N-diisopropylamino)-ethoxy, 1,2,2,6,6-pentamethyl-piperazin-4-yloxy, 2H-tetrahydropyran-4-yloxy, cyclohexyloxy, cyclopropylmethoxy, cyclopentyloxy, N-isopropylpiperadin-4-yloxy, 3-cyclopentylpropoxy, 2-oxo-propoxy, 2-hydroxy-propoxy, and (1R,3R,5S)-8-methyl-8-azabicyclo[3.2.1]octan-3-yloxy.

In a particular embodiment, the present invention provides compounds having a structural formula (I), or pharmaceutically acceptable salts thereof, as recited above wherein:
X is CH;
Ring B is pyridinyl;
R¹ is C₁₋₄alkyl;
R² is hydrogen;
R³ is a thiazolyl; wherein the thiazolyl may be optionally substituted on carbon by one or more R¹⁰;
R⁵ is selected from the group consisting of 1,3,4-oxadiazolyl, 1H-tetrazolyl, 1,3,4-thiadiazolyl, 1H-1,2,4-triazolyl, 1,2,4-oxadiazolyl, 4,5-dihydro-oxazolyl, 1H-pyrazolyl, 2-oxo-3H-1,2,3,5-oxathiadiazolyl, 1H-imidazolyl, and morpholinyl; wherein the 1,3,4-oxadiazolyl, 1H-tetrazolyl, 1,3,4-thiadiazolyl, 1H-1,2,4-triazolyl, 1,2,4-oxadiazolyl, 4,5-dihydro-oxazolyl, 1H-pyrazolyl, 2-oxo-3H-1,2,3,5-oxathiadiazolyl, 1H-imidazolyl, and morpholinyl may be optionally substituted on one or more carbon atoms with one or more R¹⁴; and wherein the —NH— moiety of the 1H-tetrazolyl, 1H-pyrazolyl, 1H-imidazolyl, morpholinyl, or the 1H-1,2,4-triazolyl may be optionally substituted by methyl;
R¹⁰ is trifluoromethylpyridinyl, phenyl, 1-methyl-1H-pyrazolyl;
m is 0; and
p is 0.

In a particular embodiment, the present invention provides compounds having a structural formula (I), or pharmaceutically acceptable salts thereof, as recited above wherein:
X is CH;
Ring B is pyridinyl;
R¹ is C₁₋₄alkyl;
R² is hydrogen;
R³ is a thiazolyl; wherein the thiazolyl may be optionally substituted on carbon by one or more R¹⁰;
R⁵ is selected from the group consisting of 5-oxo-4,5-dihydro-1,3,4-oxadiazolyl-2-yl, wherein the 5-oxo-4,5-dihydro-1,3,4-oxadiazolyl-2-yl;
R¹⁰ is trifluoromethylpyridinyl, phenyl, 1-methyl-1H-pyrazolyl;
m is 0; and
p is 0.

In a particular embodiment, the present invention provides compounds having a structural formula (I), or pharmaceutically acceptable salts thereof, as recited above wherein:
X is CH;
Ring B is pyridinyl;
R¹ is C₁₋₄alkyl;
R² is hydrogen;
R³ is a thiazolyl; wherein the thiazolyl may be optionally substituted on carbon by one or more R¹⁰;
R⁵ is selected from the group consisting of 1,3,4-oxadiazolyl, wherein the 1,3,4-oxadiazolyl, may be optionally substituted on one or more carbon atoms with one or more R¹⁴;
R¹⁰ is trifluoromethylpyridinyl, phenyl, 1-methyl-1H-pyrazolyl;
m is 0; and
p is 0.

In a particular embodiment, the present invention provides compounds having a structural formula (I), or pharmaceutically acceptable salts thereof, as recited above wherein:
X is CH;
Ring B is pyridinyl;
p is 1;
R¹ is C₁₋₄alkyl;
R² is hydrogen;
R³ is a thiazolyl; wherein the thiazolyl may be optionally substituted on carbon by one or more R¹⁰;
R⁵ is hydrogen;
R⁶ is sulfamoyl, mesyl, cyano, or halo;
R¹⁰ is trifluoromethylpyridinyl, phenyl, 1-methyl-1H-pyrazolyl; and
m is 0.

In a particular embodiment, the present invention provides compounds having a structural formula (I), or pharmaceutically acceptable salts thereof, as recited above wherein:
X is CH;
Ring B is pyridinyl, quinoxalinyl or 5,6-dihydro[1,3]thiazolo[4,5-d]pyridazine-4,7-dione;
R¹ is C₁₋₄alkyl;
R² is hydrogen;
R³ is a thiazolyl; wherein the thiazolyl may be optionally substituted on carbon by one or more R¹⁰;
R⁵ is hydrogen;
R¹⁰ is trifluoromethylpyridinyl, phenyl, 1-methyl-1H-pyrazolyl;
m is 0; and
p is 0.

In a particular embodiment, the present invention provides compounds having a structural formula (I), or pharmaceutically acceptable salts thereof, as recited above wherein:
X is CH;
Ring B is pyridin-3-yl;
p is 1;
R¹ is C₁₋₄alkyl;
R² is hydrogen;
R³ and R¹⁰ together are a 4-trifluoromethyl-thiazole-2-yl;
R⁵ is 5-oxo-4,5-dihydro-1,3,4-oxadiazolyl-2-yl;
R⁶ is sulfamoyl, mesyl, cyano, or halo; and
m is 0.

Particular compounds of the invention are the compounds of the Examples, and pharmaceutically acceptable salts thereof, each of which provides a further independent aspect of the invention. In further aspects, the present invention also comprises any two or more compounds of the Examples.

In another embodiment, the invention provides pharmaceutical compositions comprising a pharmaceutically acceptable excipient or carrier and a compound represented by formula (I), or a pharmaceutically acceptable salt thereof.

In a further aspect the present invention provides a process for preparing a compound of formula (I), or a pharmaceutically-acceptable salt thereof, wherein variable groups in the schemes below are as defined in formula (I) unless otherwise specified. In general, the compounds of the invention can be prepared by a palladium catalyzed Suzuki coupling reaction of a boronic ester derivative (i) or (iv) and a halo derivative (ii) or (iii), as shown in Schemes I and II. Typically, the coupling reaction is heated and is carried out in the presence of a base such as Cs₂CO₃.

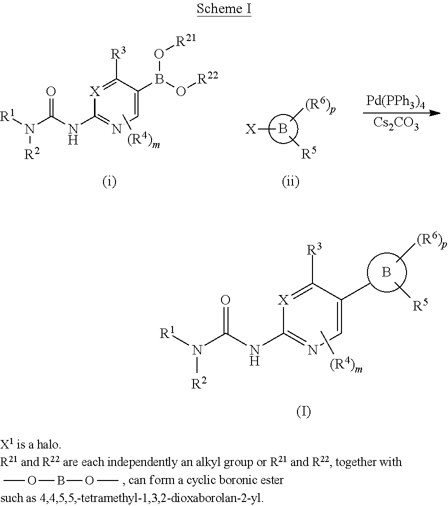

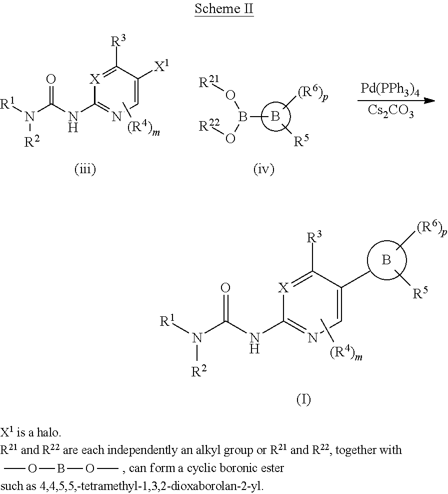

Boronic ester derivatives can be prepared by heating a halo derivative with a diboron compound such as 4,4,4′,4′,5,5,5′,5′-octamethyl-2,2′-bi(1,3,2-dioxaborolane) in the presence of 1,1′-bis(diphenylphosphino)ferrocene-palladium dichloride in an organic solvent.

The urea portion of the compounds of the invention can be prepared from an isocyanate derivative and an amine derivative either before or after the Suzuki coupling reaction (as shown in Schemes I and II). If the Suzuki coupling reaction is preformed before formation of the urea, the amine is protected with an amine protecting group. When forming the urea derivative, the isocyanate derivative (vi) is typically combined with the amine derivative (v) in an organic solvent and heated, as shown in Scheme III. The solvent can be aqueous, organic or a mixture of an aqueous miscible organic solvent and water.

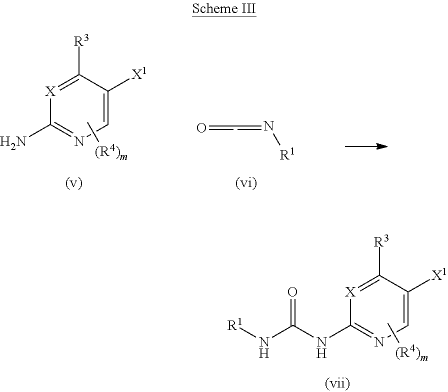

When R³ is an aryl or a heteroaryl, a Suzuki coupling reaction can be used to attach it to the pyridinyl or pyrimidinyl center ring as shown in Scheme IV. Although Scheme IV shows the coupling reaction of R³ occurring before the coupling reaction to attach ring B, the reactions could be preformed in the alternative order. When the R³ group is attached before the coupling reaction to attach ring B, the center ring the ring can be brominated by heating it with 1-bromopyrrolidine-2,5-dione to form a substrate for the Suzuki coupling reaction shown in Scheme II.

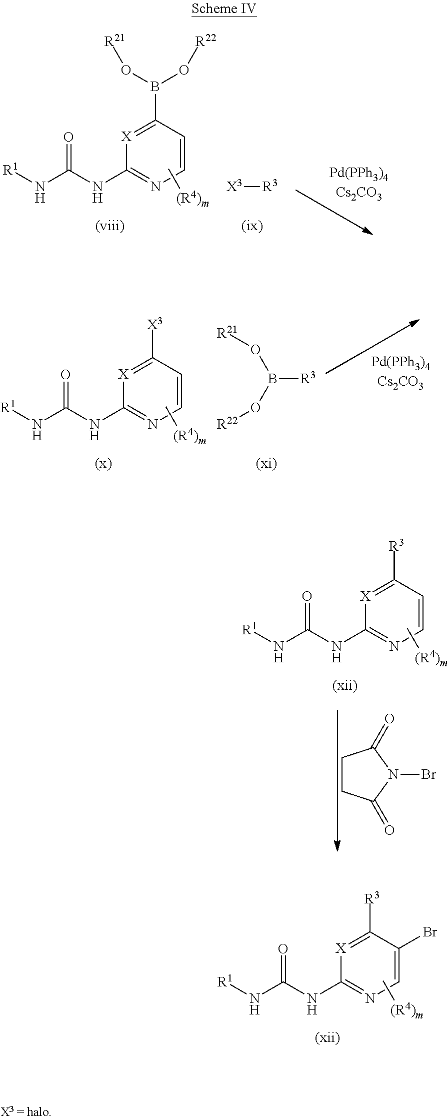

In general, when R⁵ is a heteroaryl, it can be added by a Suzuki coupling reaction analogous to that shown for R³. Likewise, R⁵ can be coupled to ring B either before or after ring B is coupled to the pyridinyl or pyrimidinyl center ring.

Alternatively, when R³ or R⁵ is a heterocyclyl, it can be prepared from an ester derivative either before or after coupling of ring B to the pyridinyl or pyrimidinyl center ring. For example, when R³ is a thiazolyl group, an ester derivative (xiii) can be converted to an amide (xiv) by treating it with a solution of ammonia in an alcohol. The amide derivative (xiv) can then be converted to a thioamide (xv) by treating the amide with Lawessons reagent. The thioamide (xv) is then heated with an α-halo-ketone or an α-halo-aldehyde (xvi) followed by treatment with an acid such as trifluoroacetic acid to form the thiazole (xvii) (see Scheme V). Although the thiazole ring is prepared before the Suzuki coupling reaction to attach ring B in Scheme V, it could also be prepared after the coupling reaction from the ester derivative. When R⁵ is a thiazolyl group, it can be prepared in an analogous manner either before or after coupling of ring B.

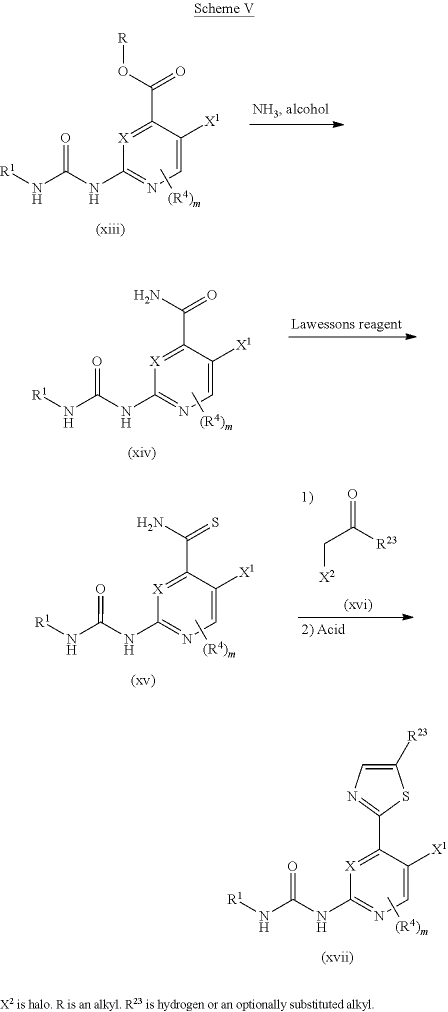

When R³ or R⁵ is tetrazolyl, it can be prepared by heating a cyano derivative with sodium azide and ammonium chloride in a solvent as shown in Scheme VI for an R⁵ tetrazolyl group. When R³ is a tetrazolyl group it can be prepared in an analogous manner to that shown in Scheme VI. In addition R³ or R⁵ tetrazolyl groups can be prepared by the reaction shown in Scheme VI either before or after coupling of ring B to the pyridinyl or pyrimidinyl center ring.

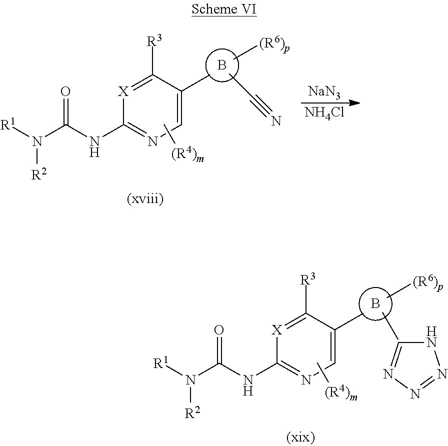

When R³ or R⁵ is a 1,3,4-oxadiazolyl group, it can be prepared from an ester derivative (xx) by treating the ester with a base in to form a carboxylic acid (xxi). The carboxylic acid (xxi) is then coupled to a hydrazide derivative (xxii) in the presence of the amide coupling reagent HATU to form a dihydrazide derivative (xxiii). The dihydrazide (xxiii) is then treated with triphenyl phosphine in an aprotic organic solvent in the presence of an excess amount of an aprotic base to form a compound of the invention in which the R⁵ group is 1,3,4-oxadiazolyl (xxiv) as shown in Scheme VII. When R³ is a 1,3,4-oxadiazolyl group it can be prepared in an analogous manner to that shown in Scheme VII. In addition R³ or R⁵ 1,3,4-oxadiazolyl groups can be prepared by the reaction shown in Scheme VII either before or after coupling of ring B to the pyridinyl or pyrimidinyl center ring.

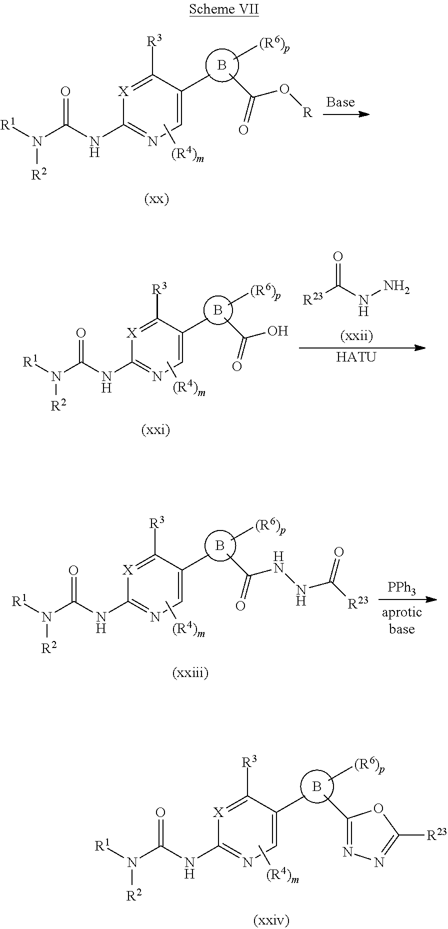

When R³ or R⁵ is a 1,3,4-thiadiazolyl group, it can be prepared from a dihydrazide derivative (xxiii) (see Scheme VII for preparation of dihydrazide derivatives). The dihydrazide derivative (xxiii) is heated with phosphorous pentasulfide and hexamethyldisiloxane in an organic solvent to form a compound of the invention having an R⁵ 1,3,4-thiadiazolyl group (xxv) as shown in Scheme VIII. When R³ is a 1,3,4-thiadiazolyl group it can be prepared in an analogous manner to that shown in Scheme VIII. In addition R³ or R⁵ 1,3,4-thiadiazolyl groups can be prepared by the reaction shown in Scheme VIII either before or after coupling of ring B to the pyridinyl or pyrimidinyl center ring.

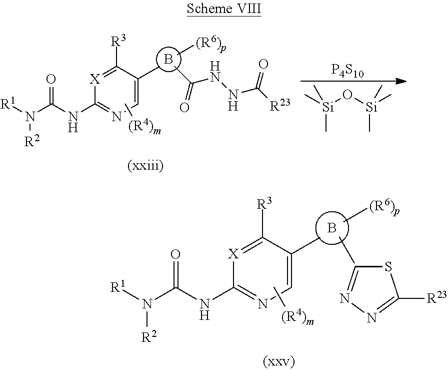

When R³ or R⁵ is a 5-oxo-4,5-dihydro-1,3,4-oxadiazol-2-yl group, it can be prepared from a carboxylic acid (xxi) or an ester (x) (see Scheme VII for preparation of the carboxylic acid derivative). The carboxylic acid (xxi) or ester (x) derivative is heated with hydrazine hydrate in an alcohol to form a hydrazide derivative (xxvi). The hydrazide derivative (xxvi) is then reacted with carbonyl diimidazole (xxvii) in the presence of an aprotic base in an aprotic solvent to form a compound of the invention which has an R⁵ is 5-oxo-4,5-dihydro-1,3,4-oxadiazol-2-yl (xxviii) as shown in Scheme IX. When R³ is a 5-oxo-4,5-dihydro-1,3,4-oxadiazol-2-yl group it can be prepared in an analogous manner to that shown in Scheme IX. In addition, R³ or R⁵⁵-oxo-4,5-dihydro-1,3,4-oxadiazol-2-yl groups can be prepared by the reaction shown in Scheme IX either before or after coupling of ring B to the pyridinyl or pyrimidinyl center ring.

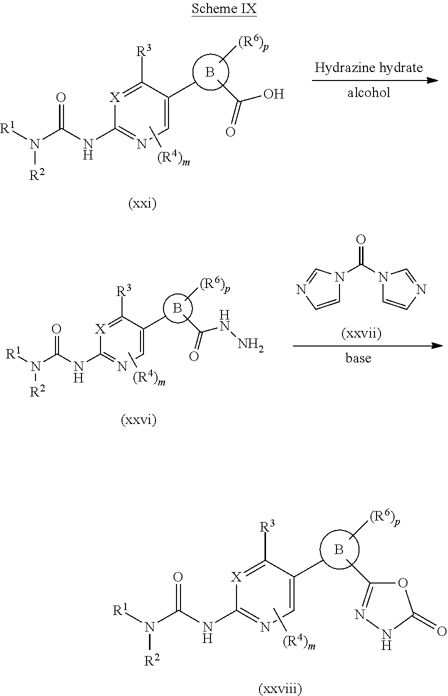

When R³ or R⁵ is a 1,2,4-triazolyl group, it can be prepared from an amide derivative (xxix) by heating it in 1-(N,N-dimethylamino)-1,1-dimethoxy-ethane (xxx) to form (xxxi) (xxxi) is then heated with acetohydrazide in acetic acid to form a compound of the invention that has an R⁵ 1,2,4-triazolyl group (xxxii) as shown in Scheme X. When R³ is a 1,2,4-triazolyl group it can be prepared in an analogous manner to that shown in Scheme X. In addition, R³ or R⁵ 1,2,4-triazolyl groups can be prepared by the reaction shown in Scheme X either before or after coupling of ring B to the pyridinyl or pyrimidinyl center ring.

When R³ or R⁵ is a 1,2,4-oxadiazolyl group, it can be prepared from (xxxi) by heating (xxxi) with hydroxylamine hydrochloride in a solution of sodium hydroxide in 70% acetic acid in dioxane to form a compound of the invention in which R⁵ is a 1,2,4-oxadiazolyl group (xxxiii) as shown in Scheme X. When R³ is a 1,2,4-oxadiazolyl group it can be prepared in an analogous manner to that shown in Scheme X. In addition, R³ or R⁵ 1,2,4-oxadiazolyl groups can be prepared by the reaction shown in Scheme X either before or after coupling of ring B to the pyridinyl or pyrimidinyl center ring.

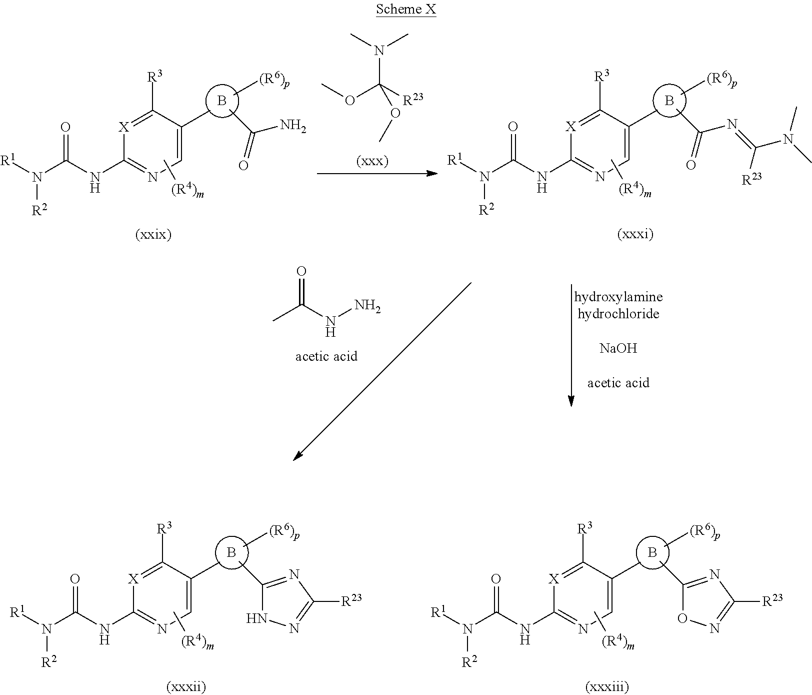

When R³ or R⁵ is an imidazolyl group, it can be prepared from a cyano derivative (xvii) by stirring the cyano derivative (xvii) at room temperature in a solution of sodium methoxide in methanol for several hours. 1,1-Dimethoxy-2-aminoethane (xxxiv) is then added to the solution and it is heated to give a compound of the invention in which R⁵ is an imidazolyl group (xxxv) as shown in Scheme XI. When R³ is an imidazolyl group it can be prepared in an analogous manner to that shown in Scheme XI. In addition, R³ or R⁵ imidazolyl groups can be prepared by the reaction shown in Scheme XI either before or after coupling of ring B to the pyridinyl or pyrimidinyl center ring.

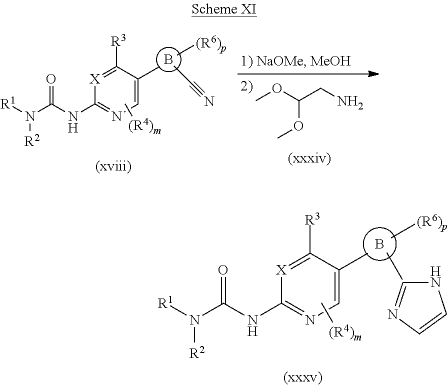

The formation of a pharmaceutically-acceptable salt is within the skill of an ordinary organic chemist using standard techniques.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. The reagents used to introduce such ring substituents are either commercially available or are made by processes known in the art.

Introduction of substituents into a ring may convert one compound of the formula (I) into another compound of the formula (I). Such reactions and modifications include, for example, introduction of a substituent by means of an aromatic substitution reaction, reduction of substituents, alkylation of substituents, oxidation of substituents, esterification of substituents, amidation of substituents, formation of heteroaryl rings. The reagents and reaction conditions for such procedures are well known in the chemical art. Particular examples of aromatic substitution reactions include the introduction of alkoxides, diazotization reactions followed by introduction of thiol group, alcohol group, halogen group. Examples of modifications include; oxidation of alkylthio to alkylsulphinyl or alkylsulphonyl.

The skilled organic chemist will be able to use and adapt the information contained and referenced within the above references, and accompanying Examples therein and also the Examples herein, to obtain necessary starting materials, and products. If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples. It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon, or for example, an allyl group which may be removed, for example, by use of a palladium catalyst such as palladium acetate.

The protecting groups may be removed at any convenient stage in the synthesis using conventional techniques well known in the chemical art, or they may be removed during a later reaction step or work-up.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

### Enzyme Potency Testing Methods

Compounds were tested for inhibition of GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays were performed in multiwell plates in 100 μl reactions containing: 50 mM TRIS buffer pH 7.5, 75 mM ammonium acetate, 5.5 mM magnesium chloride, 0.5 mM ethylenediaminetetraacetic acid, 5% glycerol, 1 mM 1,4-Dithio-DL-threitol, 200 nM bovine serum albumin, 16 μg/ml sheared salmon sperm DNA, 4 nM E. coli GyrA, 4 nM E. coli GyrB, 250 μM ATP, and compound in dimethylsulfoxide. Reactions were quenched with 150 μl of ammonium molybdate/malachite green detection reagent containing 1.2 mM malachite green hydrochloride, 8.5 mM ammonium molybdate tetrahydrate, and 1 M hydrochloric acid. Plates were read in an absorbance plate reader at 625 nm and percent inhibition values were calculated using dimethylsulfoxide (2%)-containing reactions as 0% inhibition and novobiocin-containing (2 μM) reactions as 100% inhibition controls. Compound potency was based on IC₅₀ measurements determined from reactions performed in the presence of 10 different compound concentrations.

Compounds were tested for inhibition of topoisomerase IV ATPase activity as described above for GyrB except the 100 μl reactions contained the following: 20 mM TRIS buffer pH 8, 50 mM ammonium acetate, 8 mM magnesium chloride, 5% glycerol, 5 mM 1,4-Dithio-DL-threitol, 0.005% Brij-35, 5 μg/ml sheared salmon sperm DNA, 10 nM E. coli ParC, 10 nM E. coli ParE, 160 μM ATP, and compound in dimethylsulfoxide. Compound potency was based on IC₅₀ measurements determined from reactions performed in the presence of 10 different compound concentrations.

Compounds of the invention generally have IC₅₀ values of <200 μg/ml in one or both assays described herein above.

Compounds were tested for inhibition of GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays were performed in multiwell plates in 100 μl reactions containing: 50 mM Hepes buffer pH 7.5, 75 mM ammonium acetate, 8.0 mM magnesium chloride, 1.0 mM ethylenediaminetetraacetic acid, 5% glycerol, 2 mM 1,4-Dithio-DL-threitol, 400 nM bovine serum albumin, 5 μg/ml sheared salmon sperm DNA, 1.25 nM E. coli GyrA, 1.25 nM S. aureus GyrB, 500 μM ATP, and compound in dimethylsulfoxide. Reactions were quenched with 150 μl of ammonium molybdate/malachite green detection reagent containing 1.2 mM malachite green hydrochloride, 8.5 mM ammonium molybdate tetrahydrate, and 1 M hydrochloric acid. Plates were read in an absorbance plate reader at 650 nm and percent inhibition values were calculated using dimethylsulfoxide (2%)-containing reactions as 0% inhibition and novobiocin-containing (2 μM) reactions as 100% inhibition controls. Compound potency was based on IC₅₀ measurements determined from reactions performed in the presence of 10 different compound concentrations.

Table 1 shows S. aureus (SAU) GyrB ATPase IC₅₀ values for representative compounds of the invention.

TABLE 1

| Example | IC₅₀ |
|---|---|
| Number | (μm) |
| 14 | 0.010 |
| 17 | 0.010 |
| 25 | 0.003 |
| 32 | 0.062 |

Table 2 shows S. aureus (SAU) GyrB ATPase percent inhibition for compounds of the invention at a compound concentration of 1.0 μM unless otherwise noted. Where the assay was carried out more than one time for a particular compound of the invention, the percent inhibition shown in Table 2 is an average value.

TABLE 2

| Example | % Inhibition |
|---|---|
| Number | (μm) |
| 1 | 99 |
| 2 | 88 |
| 3 | 88 |
| 4 | 99 |
| 5 | 112 |
| 6 | 106 |
| 7 | 90 |
| 8 | 95 |
| 9 | 106 |
| 10 | 95 |
| 11 | 107 |
| 12 | 108 |
| 13 | 103 |
| 14 | 86 |
| 15 | 93 |
| 16 | 115 |
| 17 | 102.2 |
| 18 | 113.0 |
| 19 | 109.9 |
| 20 | 110.6 |
| 21 | No data |
| 22 | 114 |
| 23 | 110 |
| 24 | 109 |
| 25 | 100 |
| 26 | 105 |
| 27 | 109 |
| 28 | 70 |
| 29 | 114 |
| 30 | 105 |
| 31 | 113 |
| 32 | 106 |
| 33 | 117 |
| 34 | 93 |
| 35 | 103 |
| 36 | 107 |
| 37 | 112 |
| 38 | 108 |
| 39 | 102 |
| 40 | 117 |
| 41 | 109 |
| 42 | 106 |
| 43 | No data |
| 44 | 96 |
| 45 | 103 |
| 46 | −1 |
| 47 | 72 |
| 48 | 95 |
| 49 | 99 |
| 50 | 105 |
| 51 | 98 |
| 52 | 108 |
| 53 | 109 |
| 54 | 97 |
| 55 | 96 |
| 56 | 96 |
| 57 | 96 |
| 58 | 97 |
| 59 | 98 |
| 60 | 92 |
| 61 | 95 |
| 62 | 86 |
| 63 | 96 |
| 64 | 98 |
| 65 | 94 |
| 66 | 93 |
| 67 | 93 |
| 68 | 96 |
| 69 | 91 |
| 70 | 96 |
| 71 | 93 |
| 72 | 95 |
| 73 | 97 |
| 74 | 94 |
| 75 | 99 |
| 76 | 100 |
| 77 | 97 |
| 78 | 99 |
| 79 | 95 |
| 80 | 96 |
| 81 | 94 |
| 82 | 86 |
| 83 | 94 |
| 84 | No data |
| 85 | 93 |
| 86 | 97 |
| 87 | 99 |
| 88 | 94 |
| 89 | 87 |
| 90 | 116 |
| 91 | No data |
| 92 | 104 |
| 93 | No data |
| 94 | 98 |
| 95 | 99 |
| 96 | 100 |
| 97 | 99 |
| 98 | 100 |
| 99 | 97 |
| 100 | 98 |
| 101 | 97 |
| 102 | 92 |
| 103 | 86 |
| 104 | 98 |
| 105 | 101 |
| 106 | 102 |
| 107 | 97 |
| 108 | 103 |
| 109 | 98 |
| 110 | 95 |
| 111 | 106 |
| 112 | 95 |
| 113 | 45 |
| 114 | 97 |
| 115 | 96 |
| 116 | 90 |
| 117 | 105 |
| 118 | 118 |
| 119 | 96 |
| 120 | 118 |
| 121 | No data |
| 122 | 102 |
| 123 | 78 |
| 124 | No data |
| 125 | 103 |
| 126 | 102 |
| 127 | 100 |
| 128 | 92 |
| 129 | 102 |
| 130 | 103 |
| 131 | 93 |
| 132 | 92 |
| 133 | 104 |
| 134 | 120 |
| 135 | 101 |
| 136 | 102 |
| 137 | 101 |
| 138 | 104 |
| 139 | 103 |
| 140 | 97 |
| 141 | 97 |
| 142 | 96 |
| 143 | 98 |
| 144 | 90 |
| 145 | 100 |
| 146 | 97 |
| 147 | 95 |
| 148 | 97 |
| 149 | 94 |
| 150 | 96 |
| 151 | 97 |
| 152 | 95 |
| 153 | 100 |
| 154 | 105 |
| 155 | 96 |
| 156 | 95 |
| 157 | 96 |
| 158 | 97 |
| 159 | 97 |
| 160 | 98 |
| 161 | 97 |
| 162 | 98 |
| 163 | 96 |
| 164 | 96 |
| 165 | 98 |
| 166 | 72 |
| 167 | 96 |
| 168 | 69 |
| 169 | 96 |
| 170 | No data |
| 171 | 106 |
| 172 | No data |
| 173 | 95 |
| 174 | No data |
| 175 | 93 |
| 176 | 98 |
| 177 | 86 |
| 178 | 97 |
| 179 | 101 |
| 180 | 96 |
| 181 | 97 |
| 182 | 99 |
| 183 | 96 |
| 184 | No data |
| 185 | 99 |
| 186 | 110 |
| 187 | 100 |
| 188 | 97 |
| 189 | 103 |
| 190 | 100 |
| 191 | 99 |
| 192 | 101 |
| 193 | 89 |
| 194 | 101 |
| 195 | 98 |
| 196 | 118 |
| 197 | 106 |
| 198 | 104 |
| 199 | 94 |
| 200 | 93 |
| 201 | 107 |
| 202 | 43 |
| 203 | 104 |
| 204 | 101 |
| 205 | 100 |
| 206 | 99 |
| 207 | 101 |
| 208 | 100 |
| 209 | 95 |
| 210 | 65 |
| 211 | 109 |
| 212 | 97 |
| 213 | 95 |
| 214 | 109 |
| 215 | 96 |
| 216 | 96 |
| 217 | 95 |
| 218 | 99 |
| 219 | 97 |
| 220 | 91 |
| 221 | 99 |
| 222 | 97 |
| 223 | 95 |
| 224 | 94 |
| 225 | 117 |
| 226 | 109 |
| 227 | 100 |
| 228 | 93 |
| 229 | 99 |
| 230 | 91 |
| 231 | 96 |
| 232 | 99 |
| 233 | 100 |
| 234 | 105 |
| 235 | 101 |
| 236 | 109 |
| 237 | 110 |
| 238 | 96 |
| 239 | 94 |
| 240 | 95 |
| 241 | 97 |
| 242 | 118 |
| 243 | 122 |
| 244 | 96 |
| 245 | No data |
| 246 | No data |
| 247 | 100 |
| 248 | 104 |
| 249 | 97 |
| 250 | 101 |
| 251 | 99 |
| 252 | 99 |
| 253 | No data |
| 254 | 97 |
| 255 | 98 |
| 256 | 103 |
| 257 | 102 |
| 258 | 96 |
| 259 | 95 |
| 260 | 96 |
| 261 | 96 |
| 262 | 97 |
| 263 | 100 |
| 264 | 98 |
| 265 | 101 |
| 266 | 98 |
| 267 | 98 |
| 268 | 98 |
| 269 | No data |
| 270 | 97 |
| 271 | 100 |
| 272 | 90 |
| 273 | 98 |
| 274 | 99 |
| 275 | 98 |
| 276 | 98 |
| 277 | No data |
| 278 | 68 |
| 279 | No data |
| 280 | 95 |
| 281 | 94 |
| 282 | 96 |
| 283 | 94 |
| 284 | No data |
| 285 | No data |
| 286 | No data |
| 287 | 80 |
| 288 | 91 |

### Bacterial Susceptibility Testing Methods

Compounds were tested for antimicrobial activity by susceptibility testing in liquid media. Compounds were dissolved in dimethylsulfoxide and tested in 10 doubling dilutions in the susceptibility assays. The organisms used in the assay were grown overnight on suitable agar media and then suspended in a liquid medium appropriate for the growth of the organism. The suspension was a 0.5 McFarland and a further 1 in 10 dilution was made into the same liquid medium to prepare the final organism suspension in 100 μL. Plates were incubated under appropriate conditions at 37° C. for 24 hrs prior to reading. The Minimum Inhibitory Concentration was determined as the lowest drug concentration able to reduce growth by 80% or more.

Example 14 had an MIC of 0.39 uM against Streptococcus pneumoniae.

According to a further feature of the invention there is provided a compound of the formula (I), or a pharmaceutically-acceptable salt thereof, for use in a method of treatment of the human or animal body by therapy.

In one embodiment, the invention provides a method of treating a bacterial infection in an animal, such as a human, comprising administering to the animal or human an effective amount of a compound of any one of formulas (I), or a pharmaceutically acceptable salt thereof.

We have found that compounds of the present invention inhibit bacterial DNA gyrase and/or topoisomerase IV and are therefore of interest for their antibacterial effects. In one aspect of the invention the compounds of the invention inhibit bacterial DNA gyrase and are therefore of interest for their antibacterial effects. In one aspect of the invention, the compounds of the invention inhibit topoisomerase IV and are therefore of interest for their antibacterial effects. In one aspect of the invention, the compounds of the invention inhibit both DNA gyrase and topoisomerase IV and are therefore of interest for their antibacterial effects. Thus, the compounds of the invention are useful in treating or preventing bacterial infections.

In one aspect of the invention an “infection” or “bacterial infection” refers to an infection caused by Acinetobacter baumanii. In one aspect of the invention an “infection” or “bacterial infection” refers to an infection caused by Acinetobacter haemolyticus. In one aspect of the invention an “infection” or “bacterial infection” refers to an infection caused by Acinetobacter junii. In one aspect of the invention an “infection” or “bacterial infection” refers to an infection caused by Acinetobacter johnsonii. In one aspect of the invention an “infection” or “bacterial infection” refers to an infection caused by Acinetobacter lwoffi. In one aspect of the invention an “infection” or “bacterial infection” refers to an infection caused by Bacteroides bivius. In one aspect of the invention an “infection” or “bacterial infection” refers to an infection caused by Bacteroides fragilis. In one aspect of the invention an “infection” or “bacterial infection” refers to an infection caused by Burkholderia cepacia. In one aspect of the invention an “infection” or “bacterial infection” refers to an infection caused by Campylobacter jejuni. In one aspect of the invention an “infection” or “bacterial infection” refers to an infection caused by Chlamydia pneumoniae. In one aspect of the invention an “infection” or “bacterial infection” refers to an infection caused by Chlamydia urealyticus. In one aspect of the invention an “infection” or “bacterial infection” refers to an infection caused by Chlamydophila pneumoniae. In one aspect of the invention an “infection” or “bacterial infection” refers to an infection caused by Clostridium difficile. In one aspect of the invention an “infection” or “bacterial infection” refers to an infection caused by Enterobacter aerogenes. In one aspect of the invention an “infection” or “bacterial infection” refers to an infection caused by Enterobacter cloacae. In one aspect of the invention an “infection” or “bacterial infection” refers to an infection caused by Enterococcus faecalis. In one aspect of the invention an “infection” or “bacterial infection” refers to an infection caused by Enterococcus faecium. In one aspect of the invention an “infection” or “bacterial infection” refers to an infection caused by Escherichia coli. In one aspect of the invention an “infection” or “bacterial infection” refers to an infection caused by Gardnerella vaginalis. In one aspect of the invention an “infection” or “bacterial infection” refers to an infection caused by Haemophilus parainfluenzae. In one aspect of the invention an “infection” or “bacterial infection” refers to an infection caused by Haemophilus influenzae. In one aspect of the invention an “infection” or “bacterial infection” refers to an infection caused by Helicobacter pylori. In one aspect of the invention an “infection” or “bacterial infection” refers to an infection caused by Klebsiella pneumoniae. In one aspect of the invention an “infection” or “bacterial infection” refers to an infection caused by Legionella pneumophila. In one aspect of the invention an “infection” or “bacterial infection” refers to an infection caused by Methicillin-resistant Staphylococcus aureus. In one aspect of the invention an “infection” or “bacterial infection” refers to an infection caused by Methicillin-susceptible Staphylococcus aureus. In one aspect of the invention an “infection” or “bacterial infection” refers to an infection caused by Moraxella catarrhalis. In one aspect of the invention an “infection” or “bacterial infection” refers to an infection caused by Morganella morganii. In one aspect of the invention an “infection” or “bacterial infection” refers to an infection caused by Mycoplasma pneumoniae. In one aspect of the invention an “infection” or “bacterial infection” refers to an infection caused by Neisseria gonorrhoeae. In one aspect of the invention an “infection” or “bacterial infection” refers to an infection caused by Penicillin-resistant Streptococcus pneumoniae. In one aspect of the invention an “infection” or “bacterial infection” refers to an infection caused by Penicillin-susceptible Streptococcus pneumoniae. In one aspect of the invention an “infection” or “bacterial infection” refers to an infection caused by Peptostreptococcus magnus. In one aspect of the invention an “infection” or “bacterial infection” refers to an infection caused by Peptostreptococcus micros. In one aspect of the invention an “infection” or “bacterial infection” refers to an infection caused by Peptostreptococcus anaerobius. In one aspect of the invention an “infection” or “bacterial infection” refers to an infection caused by Peptostreptococcus asaccharolyticus. In one aspect of the invention an “infection” or “bacterial infection” refers to an infection caused by Peptostreptococcus prevotii. In one aspect of the invention an “infection” or “bacterial infection” refers to an infection caused by Peptostreptococcus tetradius. In one aspect of the invention an “infection” or “bacterial infection” refers to an infection caused by Peptostreptococcus vaginalis. In one aspect of the invention an “infection” or “bacterial infection” refers to an infection caused by Proteus mirabilis. In one aspect of the invention an “infection” or “bacterial infection” refers to an infection caused by Pseudomonas aeruginosa. In one aspect of the invention an “infection” or “bacterial infection” refers to an infection caused by Quinolone-Resistant Staphylococcus aureus. In one aspect of the invention an “infection” or “bacterial infection” refers to an infection caused by Quinolone-Resistant Staphylococcus epidermis. In one aspect of the invention an “infection” or “bacterial infection” refers to an infection caused by Salmonella typhi. In one aspect of the invention an “infection” or “bacterial infection” refers to an infection caused by Salmonella paratyphi. In one aspect of the invention an “infection” or “bacterial infection” refers to an infection caused by Salmonella enteritidis. In one aspect of the invention an “infection” or “bacterial infection” refers to an infection caused by Salmonella typhimurium. In one aspect of the invention an “infection” or “bacterial infection” refers to an infection caused by Serratia marcescens. In one aspect of the invention an “infection” or “bacterial infection” refers to an infection caused by Staphylococcus aureus. In one aspect of the invention an “infection” or “bacterial infection” refers to an infection caused by Staphylococcus epidermidis. In one aspect of the invention an “infection” or “bacterial infection” refers to an infection caused by Staphylococcus saprophyticus. In one aspect of the invention an “infection” or “bacterial infection” refers to an infection caused by Streptoccocus agalactiae. In one aspect of the invention an “infection” or “bacterial infection” refers to an infection caused by Streptococcus pneumoniae. In one aspect of the invention an “infection” or “bacterial infection” refers to an infection caused by Streptococcus pyogenes. In one aspect of the invention an “infection” or “bacterial infection” refers to an infection caused by Stenotrophomonas maltophilia. In one aspect of the invention an “infection” or “bacterial infection” refers to an infection caused by Ureaplasma urealyticum. In one aspect of the invention an “infection” or “bacterial infection” refers to an infection caused by Vancomycin-Resistant Enterococcus faecium. In one aspect of the invention an “infection” or “bacterial infection” refers to an infection caused by Vancomycin-Resistant Enterococcus faecalis. In one aspect of the invention an “infection” or “bacterial infection” refers to an infection caused by Vancomycin-Resistant Staphylococcus aureus. In one aspect of the invention an “infection” or “bacterial infection” refers to an infection caused by Vancomycin-Resistant Staphylococcus epidermis. In one aspect of the invention an “infection” or “bacterial infection” refers to an infection caused by Mycobacterium tuberculosis. In one aspect of the invention an “infection” or “bacterial infection” refers to an infection caused by Clostridium perfringens. In one aspect of the invention an “infection” or “bacterial infection” refers to an infection caused by Klebsiella oxytoca. In one aspect of the invention an “infection” or “bacterial infection” refers to an infection caused by Neisseria miningitidis. In one aspect of the invention an “infection” or “bacterial infection” refers to an infection caused by Fusobacterium spp. In one aspect of the invention an “infection” or “bacterial infection” refers to an infection caused by Peptococcus spp. In one aspect of the invention an “infection” or “bacterial infection” refers to an infection caused by Proteus vulgaris. In one aspect of the invention an “infection” or “bacterial infection” refers to an infection caused by Coagulase-negative Staphylococcus (including Staphylococcus lugdunensis, Staphylococcus capitis, Staphylococcus hominis, and Staphylococcus saprophyticus).

In one aspect of the invention an “infection” or “bacterial infection” refers to an infection caused by Acinetobacter spp. In one aspect of the invention an “infection” or “bacterial infection” refers to an infection caused by Bacteroides spp. In one aspect of the invention an “infection” or “bacterial infection” refers to an infection caused by Burkholderia spp. In one aspect of the invention an “infection” or “bacterial infection” refers to an infection caused by Campylobacter spp. In one aspect of the invention an “infection” or “bacterial infection” refers to an infection caused by Chlamydia spp. In one aspect of the invention an “infection” or “bacterial infection” refers to an infection caused by Chlamydophila spp. In one aspect of the invention an “infection” or “bacterial infection” refers to an infection caused by Clostridium spp. In one aspect of the invention an “infection” or “bacterial infection” refers to an infection caused by Enterobacter spp. In one aspect of the invention an “infection” or “bacterial infection” refers to an infection caused by Enterococcus spp. In one aspect of the invention an “infection” or “bacterial infection” refers to an infection caused by Escherichia spp. In one aspect of the invention an “infection” or “bacterial infection” refers to an infection caused by Gardnerella spp. In one aspect of the invention an “infection” or “bacterial infection” refers to an infection caused by Haemophilus spp. In one aspect of the invention an “infection” or “bacterial infection” refers to an infection caused by Helicobacter spp. In one aspect of the invention an “infection” or “bacterial infection” refers to an infection caused by Klebsiella spp. In one aspect of the invention an “infection” or “bacterial infection” refers to an infection caused by Legionella spp. In one aspect of the invention an “infection” or “bacterial infection” refers to an infection caused by Moraxella spp. In one aspect of the invention an “infection” or “bacterial infection” refers to an infection caused by Morganella spp. In one aspect of the invention an “infection” or “bacterial infection” refers to an infection caused by Mycoplasma spp. In one aspect of the invention an “infection” or “bacterial infection” refers to an infection caused by Neisseria spp. In one aspect of the invention an “infection” or “bacterial infection” refers to an infection caused by Peptostreptococcus spp. In one aspect of the invention an “infection” or “bacterial infection” refers to an infection caused by Proteus spp. In one aspect of the invention an “infection” or “bacterial infection” refers to an infection caused by Pseudomonas spp. In one aspect of the invention an “infection” or “bacterial infection” refers to an infection caused by Salmonella spp. In one aspect of the invention an “infection” or “bacterial infection” refers to an infection caused by Serratia spp. In one aspect of the invention an “infection” or “bacterial infection” refers to an infection caused by Staphylococcus spp. In one aspect of the invention an “infection” or “bacterial infection” refers to an infection caused by Streptoccocus spp. In one aspect of the invention an “infection” or “bacterial infection” refers to an infection caused by Stenotrophomonas spp. In one aspect of the invention an “infection” or “bacterial infection” refers to an infection caused by Ureaplasma spp. In one aspect of the invention an “infection” or “bacterial infection” refers to an infection caused by aerobes. In one aspect of the invention an “infection” or “bacterial infection” refers to an infection caused by obligate anaerobes. In one aspect of the invention an “infection” or “bacterial infection” refers to an infection caused by facultative anaerobes. In one aspect of the invention an “infection” or “bacterial infection” refers to an infection caused by gram-positive bacteria. In one aspect of the invention an “infection” or “bacterial infection” refers to an infection caused by gram-negative bacteria. In one aspect of the invention an “infection” or “bacterial infection” refers to an infection caused by gram-variable bacteria. In one aspect of the invention an “infection” or “bacterial infection” refers to an infection caused by atypical respiratory pathogens. In one aspect of the invention an “infection” or “bacterial infection” refers to an infection caused by Enterics. In one aspect of the invention an “infection” or “bacterial infection” refers to an infection caused by Shigella spp. In one aspect of the invention an “infection” or “bacterial infection” refers to an infection caused by Citrobacter.

In one aspect of the invention “infection” or “bacterial infection” refers to a gynecological infection. In one aspect of the invention “infection” or “bacterial infection” refers to a respiratory tract infection (RTI). In one aspect of the invention “infection” or “bacterial infection” refers to a sexually transmitted disease. In one aspect of the invention “infection” or “bacterial infection” refers to a urinary tract infection. In one aspect of the invention “infection” or “bacterial infection” refers to acute exacerbation of chronic bronchitis (ACEB). In one aspect of the invention “infection” or “bacterial infection” refers to acute otitis media. In one aspect of the invention “infection” or “bacterial infection” refers to acute sinusitis. In one aspect of the invention “infection” or “bacterial infection” refers to an infection caused by drug resistant bacteria. In one aspect of the invention “infection” or “bacterial infection” refers to catheter-related sepsis. In one aspect of the invention “infection” or “bacterial infection” refers to chancroid. In one aspect of the invention “infection” or “bacterial infection” refers to chlamydia. In one aspect of the invention “infection” or “bacterial infection” refers to community-acquired pneumonia (CAP). In one aspect of the invention “infection” or “bacterial infection” refers to complicated skin and skin structure infection. In one aspect of the invention “infection” or “bacterial infection” refers to uncomplicated skin and skin structure infection. In one aspect of the invention “infection” or “bacterial infection” refers to endocarditis. In one aspect of the invention “infection” or “bacterial infection” refers to febrile neutropenia. In one aspect of the invention “infection” or “bacterial infection” refers to gonococcal cervicitis. In one aspect of the invention “infection” or “bacterial infection” refers to gonococcal urethritis. In one aspect of the invention “infection” or “bacterial infection” refers to hospital-acquired pneumonia (HAP). In one aspect of the invention “infection” or “bacterial infection” refers to osteomyelitis. In one aspect of the invention “infection” or “bacterial infection” refers to sepsis. In one aspect of the invention “infection” or “bacterial infection” refers to syphilis. In one aspect of the invention “infection” or “bacterial infection” refers to ventilator-associated pneumonia. In one aspect of the invention “infection” or “bacterial infection” refers to intraabdominal infections. In one aspect of the invention “infection” or “bacterial infection” refers to gonorrhoeae. In one aspect of the invention “infection” or “bacterial infection” refers to meningitis. In one aspect of the invention “infection” or “bacterial infection” refers to tetanus. In one aspect of the invention “infection” or “bacterial infection” refers to tuberculosis.

In one embodiment, it is expected that the compounds of the present invention will be useful in treating bacterial infections including, but not limited to community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt thereof.

According to a further feature of the invention there is provided a method for inhibition of bacterial DNA gyrase and/or topoisomerase IV in a warm-blooded animal, such as a human being, in need of such treatment which comprises administering to said animal an effective amount of a compound of formula (I), or a pharmaceutically acceptable salt thereof as defined hereinbefore.

According to a further feature of the invention there is provided a method of treating a bacterial infection in a warm-blooded animal, such as a human being, in need of such treatment which comprises administering to said animal an effective amount of a compound of formula (I), or a pharmaceutically acceptable salt thereof as defined hereinbefore.

According to a further feature of the invention there is provided a method of treating a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococciin a warm-blooded animal, such as a human being, in need of such treatment which comprises administering to said animal an effective amount of a compound of formula (I), or a pharmaceutically acceptable salt thereof as defined hereinbefore.

A further feature of the present invention is a compound of formula (I), and pharmaceutically acceptable salts thereof for use as a medicament. Suitably the medicament is an antibacterial agent.

According to a further aspect of the invention there is provided the use of a compound of formula (I), or a pharmaceutically acceptable salt thereof in the manufacture of a medicament for use in the production of an anti-bacterial effect in a warm-blooded animal such as a human being.

According to a further aspect of the invention there is provided the use of a compound of formula (I), or a pharmaceutically acceptable salt thereof, in the manufacture of a medicament for use in inhibition of bacterial DNA gyrase and/or topoisomerase IV in a warm-blooded animal such as a human being.

Thus according to a further aspect of the invention there is provided the use of a compound of formula (I), or a pharmaceutically acceptable salt thereof, in the manufacture of a medicament for use in the treatment of a bacterial infection in a warm-blooded animal such as a human being.

Thus according to a further aspect of the invention there is provided the use of a compound of formula (I), or a pharmaceutically acceptable salt thereof, in the manufacture of a medicament for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in a warm-blooded animal such as a human being.

According to a further aspect of the invention there is provided a compound of formula (I), or a pharmaceutically acceptable salt thereof, for use in the production of an anti-bacterial effect in a warm-blooded animal such as a human being.

According to a further aspect of the invention there is provided a compound of formula (I), or a pharmaceutically acceptable salt thereof, for use in inhibition of bacterial DNA gyrase and/or topoisomerase IV in a warm-blooded animal such as a human being.

Thus according to a further aspect of the invention there is provided a compound of formula (I), or a pharmaceutically acceptable salt thereof, for use in the treatment of a bacterial infection in a warm-blooded animal such as a human being.

Thus according to a further aspect of the invention there is provided a compound of formula (I), or a pharmaceutically acceptable salt thereof, for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in a warm-blooded animal such as a human being.

In order to use a compound of the formula (I), or a pharmaceutically-acceptable salt thereof, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the formula (I), or a pharmaceutically-acceptable salt thereof, and a pharmaceutically-acceptable diluent or carrier.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in producing an anti-bacterial effect in an warm-blooded animal, such as a human being.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in inhibition of bacterial DNA gyrase and/or topoisomerase IV in an warm-blooded animal, such as a human being.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in the treatment of a bacterial infection in an warm-blooded animal, such as a human being.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in an warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The compounds of the invention described herein may be applied as a sole therapy or may involve, in addition to a compound of the invention, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination. Suitable classes and substances may be selected from one or more of the following:
i) other antibacterial agents for example macrolides e.g. erythromycin, azithromycin or clarithromycin; quinolones e.g. ciprofloxacin or levofloxacin; β-lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or
ii) anti-infective agents for example, an antifungal triazole e.g. or amphotericin; and/or
iii) biological protein therapeutics for example antibodies, cytokines, bactericidal/permeability-increasing protein (BPI) products; and/or
iv) efflux pump inhibitors.

Therefore, in a further aspect of the invention there is provided a compound of the formula (I), or a pharmaceutically acceptable salt thereof, and a chemotherapeutic agent selected from:
i) one or more additional antibacterial agents; and/or
ii) one or more anti-infective agents; and/or
iii) biological protein therapeutics for example antibodies, cytokines, bactericidal/permeability-increasing protein (BPI) products; and/or
iv) one or more efflux pump inhibitors.

In another embodiment, the invention relates to a method of treating a bacterial infection in an animal, such as a human, comprising administering to the animal an effective amount of a compound of formula (I), or a pharmaceutically acceptable salt thereof, and a chemotherapeutic agent selected from:
i) one or more additional antibacterial agents; and/or
ii) one or more anti-infective agents; and/or
iii) biological protein therapeutics for example antibodies, cytokines, bactericidal/permeability-increasing protein (BPI) products; and/or
iv) one or more efflux pump inhibitors.

As stated above the size of the dose required for the therapeutic or prophylactic treatment of a particular disease state will necessarily be varied depending on the host treated, the route of administration, the severity of the illness being treated, and whether or not an additional chemotherapeutic agent is administered in combination with a compound of the invention. Preferably a daily dose in the range of 1-50 mg/kg is employed. However the daily dose will necessarily be varied depending upon the host treated, the particular route of administration, the severity of the illness being treated, and whether or not an additional chemotherapeutic agent is administered in combination with a compound of the invention. Accordingly the optimum dosage may be determined by the practitioner who is treating any particular patient.

As noted above, one embodiment of the present invention is directed to treating or preventing diseases caused by bacterial infections, wherein the bacteria comprise a GyrB ATPase or topoisomerase IV ATPase enzyme. “Treating a subject with a disease caused by a bacterial infection” includes achieving, partially or substantially, one or more of the following: the reducing or amelioration of the progression, severity and/or duration of the infection, arresting the spread of an infection, ameliorating or improving a clinical symptom or indicator associated with a the infection (such as tissue or serum components), and preventing the reoccurrence of the infection.

As used herein, the terms “preventing a bacterial infection” refer to the reduction in the risk of acquiring the infection, or the reduction or inhibition of the recurrence of the infection. In a preferred embodiment, a compound of the invention is administered as a preventative measure to a patient, preferably a human, before a surgical procedure is preformed on the patient to prevent infection.

As used herein, the term “effective amount” refers to an amount of a compound of this invention for treating or preventing a bacterial infection is an amount which is sufficient to prevent the onset of an infection, reduce or ameliorate the severity, duration, or progression, of an infection, prevent the advancement of an infection, cause the regression of an infection, prevent the recurrence, development, onset or progression of a symptom associated with an infection, or enhance or improve the prophylactic or therapeutic effect(s) of another therapy.

In addition to its use in therapeutic medicine, compounds of formula (I), and their pharmaceutically acceptable salts, are also useful as pharmacological tools in the development and standardization of in-vitro and in-vivo test systems for the evaluation of the effects of inhibitors of DNA gyrase and/or topoisomerase IV in laboratory animals such as cats, dogs, rabbits, monkeys, rats and mice, as part of the search for new therapeutic agents.

In the above other, pharmaceutical composition, process, method, use and medicament manufacture features, the alternative and particular embodiments of the compounds of the invention described herein also apply.

### EXAMPLES

The invention is now illustrated but not limited by the following Examples in which unless otherwise stated:—

(i) evaporations were carried out by rotary evaporation in-vacuo and work-up procedures were carried out after removal of residual solids by filtration;
(ii) operations were generally carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable; the structure of the end-products of the invention were generally confirmed by NMR and mass spectral techniques; proton magnetic resonance spectra is quoted and was generally determined in DMSO-d₆ unless otherwise stated using a Bruker DRX-300 spectrometer operating at a field strength of 300 MHz. Chemical shifts are reported in parts per million downfield from tetramethysilane as an internal standard (δ scale) and peak multiplicities are shown thus: s, singlet; d, doublet; AB or dd, doublet of doublets; dt, doublet of triplets; dm, doublet of multiplets; t, triplet, m, multiplet; br, broad; fast-atom bombardment (FAB) mass spectral data were generally obtained using a Platform spectrometer (supplied by Micromass) run in electrospray and, where appropriate, either positive ion data or negative ion data were collected or using Agilent 1100 series LC/MSD equipped with Sedex 75ELSD, run in atmospheric pressure chemical ionisation mode and, where appropriate, either positive ion data or negative ion data were collected; mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported;
(vi) each intermediate was purified to the standard required for the subsequent stage and was characterised in sufficient detail to confirm that the assigned structure was correct; purity was assessed by high pressure liquid chromatography, thin layer chromatography, or NMR and identity was determined by infra-red spectroscopy (IR), mass spectroscopy or NMR spectroscopy as appropriate;
(vii) the following abbreviations may be used:
ACN is acetonitrile;
CDCl₃ is deuterated chloroform;
CDI is 1,1′-carbonyl diimidazole;
DBU is 1,8-diazabicyclo[5.4.0]undec-7-ene;
DCM is dichloromethane;
DIEA is diisopropyl ethylamine;
DMAP is N,N-dimethylaminopyridine;
DMF is N,N-dimethylformamide;
DMSO is dimethylsulfoxide;
EDC is 1-ethyl-3-(3-dimethylaminopropyl)carbodiimide;
EtOAc is ethyl acetate;
EtOH is ethanol;
HATU is N-[(dimethylamino)-1H,2,3-triazolo[4,5-b-]pyridin-1-ylmethylene]-N-methylmethanaminium hexafluorophosphate N-oxide;
HOBT is 1-hydroxybenzotriazole;
MeOH is methanol;
MS is mass spectroscopy;
MTBE is methyl tert-butyl ether;
RT or rt is room temperature;
SM is starting material;
TBFA is tetra-n-butylammonium fluoride;
TFA is trifluoroacetic acid;
TFAA is trifluoroacetic anhydride;
THF is tetrahydrofuran;
XPhos is dicyclohexyl(2′,4′,6′-triisopropylbiphenyl-2-yl)phosphine; and
   (viii) temperatures are quoted as ° C.

### Example 1

### 1-Ethyl-3-(5′-(5-methyl-1,3,4-oxadiazol-2-yl)-4-(4-(trifluoromethyl)thiazol-2-yl)-3,3′-bipyridin-6-yl)urea

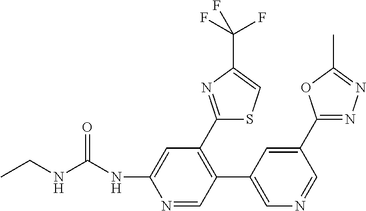

Triethylamine (0.054 mL, 0.39 mmol) and acetohydrazide (14.40 mg, 0.19 mmol) were added to a solution of 6′-(3-ethylureido)-4′-(4-(trifluoromethyl)thiazol-2-yl)-3,3′-bipyridine-5-carboxylic acid (Intermediate 1, 85 mg, 0.19 mmol) in DMF (1.5 mL). The mixture was stirred for 5 minutes and then HATU (89 mg, 0.23 mmol) was added. The resulting light yellow solution was stirred at room temperature for one hour. Then the reaction was diluted with water and the aqueous layer was lyophilized to remove water. The residue obtained was extracted with THF and concentrated to give thick oil. The oil was taken up in DCM (5 mL) and triphenyl phosphine (6 eq, 306 mg, 1.16 mmol), carbon tetrachloride (3 eq, 180 mg, 113 uL, 0.58 mmol), and triethylamine (6 eq, 319 mg, 0.431 uL, 1.16 mmol) were added and allowed to stir overnight at room temperature. The reaction was concentrated and partitioned between water and dichloromethane. The organic layer was washed with water and brine, then dried over magnesium sulfate. The crude residue was purified by normal phase chromatography to give a white solid as the product (48 mg).

MS (ES) MH⁺: 476 for C₂₀H₁₆F₃N₇O₂S

¹H-NMR (DMSO-d₆) δ: 1.09 (t, 3H); 2.58 (s, 3H); 3.16-3.24 (m, 2H); 7.54 (brs, 1H); 8.23 (s, 1H); 8.35 (s, 1H); 8.40 (s, 1H); 8.56 (s, 1H); 8.69 (s, 1H); 9.15 (d, 1H); 9.51 (s, 1H).

### Example 2

### 1-(5′-Cyano-4-(4-(trifluoromethyl)thiazol-2-yl)-3,3′-bipyridin-6-yl)-3-ethylurea

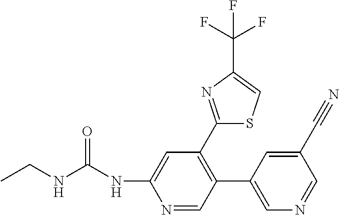

1-(5-Bromo-4-(4-(trifluoromethyl)thiazol-2-yl)pyridin-2-yl)-3-ethylurea (Intermediate 3, 300 mg, 0.76 mmol), cesium carbonate (495 mg, 1.52 mmol), tetrakis(triphenylphosphine)palladium (0) (88 mg, 0.08 mmol), and 5-(4,4,5,5-tetramethyl-1,3,2-dioxaborolan-2-yl)nicotinonitrile (349 mg, 1.52 mmol) were taken in a microwave vial and degassed with nitrogen. Then dioxane:water (4:1, 6 mL) was added to the vial and the mixture was microwaved at 100° C. for half an hour. The reaction mixture was partitioned between water and ethyl acetate and the layers were separated. The aqueous layer was back extracted with ethyl acetate (2-3 times). The combined organic layers were washed with saturated sodium bicarbonate solution, water, brine and dried over magnesium sulfate. The solvent was removed and the residue was washed with acetonitrile to give the title compound as a white solid (270 mg).

MS (ESP): 419 (M+1) for C₁₈H₁₃FN₆OS

¹H-NMR (DMSO-d₆) δ: 1.09 (t, 3H); 3.16-3.22 (m, 2H); 7.49 (t, 1H); 8.22 (s, 1H); 8.36 (s, 1H); 8.38 (d, 1H); 8.60 (s, 1H); 8.76 (s, 1H); 9.04 (s, 1H); 9.52 (s, 1H).

### Example 3

### 1-(5′-(2H-tetrazol-5-yl)-4-(4-(trifluoromethyl)thiazol-2-yl)-3,3′-bipyridin-6-yl)-3-ethylurea

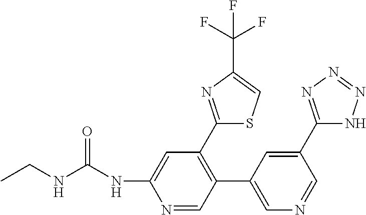

Sodium azide (18.65 mg, 0.29 mmol) and ammonium chloride (14.57 mg, 0.27 mmol) were added to a solution of 1-(5′-cyano-4-(4-(trifluoromethyl)thiazol-2-yl)-3,3′-bipyridin-6-yl)-3-ethylurea (Example 2, 60 mg, 0.14 mmol) in DMF (1.5 mL), and the mixture was heated at 100° C. for 5-6 hours. The reaction was then concentrated and dissolved in water and methanol (3 mL, 1:1) and purified by reverse phase. Fractions were collected and lyophilized to give the product as a white solid (23 mg).

MS (ESP): 462 (M+1) for C₁₈H₁₄FN₉OS

¹H-NMR (DMSO-d₆: 1.09 (t, 3H); 3.17-3.22 (m, 2H); 7.53 (t, 1H); 8.25 (s, 1H); 8.35 (s, 1H); 8.40 (s, 1H); 8.55 (s, 1H); 8.77 (d, 1H); 9.22 (s, 1H); 9.53 (s, 1H).

### Example 4

### 1-Ethyl-3-5′-5-isopropyl-1,3,4-oxadiazol-2-yl)-4-(4-trifluoromethyl)thiazol-2-yl)-3,3′-bipyridin-6-yl)urea

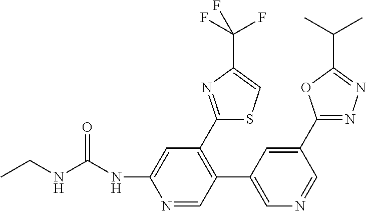

Triphenylphosphine (211 mg, 0.81 mmol), carbon tetrachloride (0.039 mL, 0.40 mmol) and triethylamine (0.112 mL, 0.81 mmol) were added to a mixture of 1-ethyl-3-(5′-(2-isobutyrylhydrazinecarbonyl)-4-(4-(trifluoromethyl)thiazol-2-yl)-3,3′-bipyridin-6-yl)urea (Intermediate 8, 70 mg, 0.13 mmol) in DCM (4 mL). The resulting mixture was allowed to stir overnight at room temperature, then was partitioned between water and dichloromethane. The layers were separated and the aqueous was back extracted with dichloromethane. The combined extract was washed with water, dried over magnesium sulfate and concentrated. The residue obtained was purified by normal phase (1% MeOH to 5% MeOH in dichloromethane) to give the product as a white solid (23 mg).

MS (ESP): 504 (M+1) for C₂₂H₂₀F₃N₇O₂S

¹H-NMR (DMSO-d₆) δ: 1.10 (t, 3H); 1.35 (d, 6H); 3.10-3.25 (m, 2H); 3.23-3.30 (m, 1H); 7.55 (brs, 1H); 8.22 (s, 1H); 8.29 (s, 1H); 8.41 (s, 1H); 8.57 (s, 1H); 8.70 (d, 1H); 9.18 (s, 1H); 9.51 (s, 1H).

### Example 5

### 1-Ethyl-3-(5′-(5-isopropyl-1,3,4-thiadiazol-2-yl)-4-(4-(trifluoromethyl)thiazol-2-yl)-3,3′-bipyridin-6-yl)urea

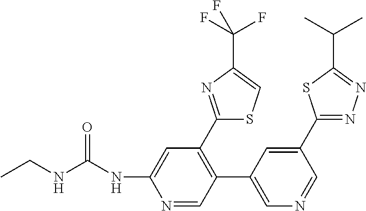

Phosphorus pentasulfide (79 mg, 0.35 mmol) and hexamethyldisiloxane (0.030 mL, 0.14 mmol) were added to a mixture of 1-ethyl-3-(5′-(2-isobutyrylhydrazinecarbonyl)-4-(4-(trifluoromethyl)thiazol-2-yl)-3,3′-bipyridin-6-yl)urea (Intermediate 8, 70 mg, 0.14 mmol) in toluene, and the mixture was refluxed overnight. The reaction was cooled to the room temperature and diluted with acetone (5 mL) and potassium carbonate (31.4 mg, 0.23 mmol) was added slowly. After the completion of the addition, the reaction was concentrated and the residue was partitioned between water and dichloromethane. The layers separated and the aqueous was back extracted with dichloromethane. The combined organic layers were washed with water, dried over magnesium sulfate and concentrated. The crude residue was purified by normal phase chromatography (1% MeOH in dichloromethane to 5% MeOH) to give the desired product (20 mg).

MS (ESP): 520 (M+1) for C₂₂H₂₀F₃N₇OS

¹H-NMR (DMSO-d₆) δ: 1.10 (t, 3H); 1.40 (d, 6H); 3.10-3.26 (m, 2H); 3.43-3.63 (m, 1H); 7.55 (brs, 1H); 8.23 (s, 1H); 8.28 (s, 1H); 8.41 (s, 1H); 8.56 (s, 1H); 8.64 (d, 1H); 9.16 (d, 1H); 9.50 (s, 1H).

### Example 6

### 1-Ethyl-3-(5′-(5-oxo-4,5-dihydro-1,3,4-oxadiazol-2-yl)-4-(4-(trifluoromethyl)thiazol-2-yl)-3,3′-bipyridin-6-yl)urea

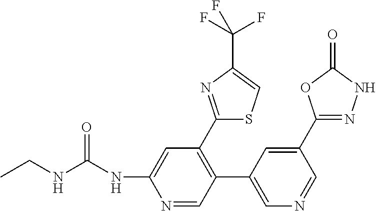

Diisopropylethylamine (0.061 mL, 0.35 mmol) and carbonyldiimidazole (56.6 mg, 0.35 mmol) were added to a solution of 1-ethyl-3-(5′-(hydrazinecarbonyl)-4-(4-(trifluoromethyl)thiazol-2-yl)-3,3′-bipyridin-6-yl)urea (Intermediate 9, 105 mg, 0.23 mmol) in DMF (1.5 mL), and the mixture was stirred at room temperature for 1.5 hours. The reaction was diluted with water and extracted with 5% methanol in dichloromethane. The combined extract was washed with water, brine, dried over magnesium sulfate and concentrated under reduced pressure. The crude residue obtained was purified by normal phase chromatography (2% MeOH in dichloromethane to 8% MeOH) to give the desired compound as a white solid (65 mg).

MS (ESP): 478 (M+1) for C₁₉H₁₄F₃N₇O₃S

¹H-NMR (DMSO-d₆) δ: 1.10 (t, 3H); 3.16-3.28 (m, 2H); 7.55 (brs, 1H); 8.09 (s, 1H); 8.22 (s, 1H); 8.37 (s, 1H); 8.57 (s, 1H); 8.62 (s, 1H); 8.97 (s, 1H); 9.50 (s, 1H); 12.80 (s, 1H).

### Example 7

### 1-Ethyl-3-(5′-(3-methyl-1H-1,2,4-triazol-5-yl)-4-(4-(trifluoromethyl)thiazol-2-yl)-3,3′-bipyridin-6-yl)urea

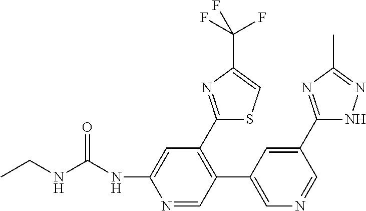

N-(1-(dimethylamino)ethylidene)-6′-(3-ethylureido)-4′-(4-(trifluoromethyl)thiazol-2-yl)-3,3′-bipyridine-5-carboxamide (Intermediate 10, 80 mg, 0.16 mmol) was added to a solution of acetohydrazide (12.90 mg, 0.17 mmol) in acetic acid (2 mL), and the resulting solution was heated at 90° C. for one hour. Then the reaction was concentrated, diluted with water and extracted with dichloromethane. During the work up process, the product started to precipitate. The mixture was washed with a 1M potassium carbonate solution twice and the precipitate was collected by filtration and dried to give the product as a white solid (35 mg).

MS (ESP): 475 (M+1) for C₂₀H₁₇F₃N₈OS

¹H-NMR (DMSO-d₆) δ: 1.10 (t, 3H); 2.31 (s, 3H); 3.12-3.26 (m, 2H); 7.74 (brs, 1H); 8.18 (s, 1H); 8.27 (s, 1H); 8.34 (s, 1H); 8.38 (s, 1H); 8.51 (s, 1H); 9.14 (d, 1H); 9.61 (s, 1H).

### Example 8

### 1-Ethyl-3-(5′43-methyl-1,2,4-oxadiazol-5-yl)-4-(4-(trifluoromethyl)thiazol-2-yl)-3,3′-bipyridin-6-yl)urea

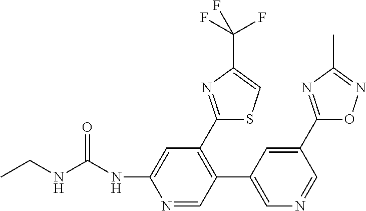

N-(1-(dimethylamino)ethylidene)-6′-(3-ethylureido)-4′-(4-(trifluoromethyl)thiazol-2-yl)-3,3′-bipyridine-5-carboxamide (Intermediate 10, 80 mg, 0.16 mmol) was added to a solution of hydroxylamine hydrochloride (13.20 mg, 0.19 mmol) in a mixture of sodium hydroxide (0.038 mL, 0.19 mmol) and 70% aq acetic acid (2 mL), and 3 ml of dioxane. The resulting mixture was slowly warmed to temperature 80° C. Most of the solid went into solution at 35° C. and solid precipitated out of solution at 50° C. The mixture was allowed to stir for 30 minutes then concentrated. The residue was partitioned between with water and dichloromethane, the layers separated and the aqueous layer was back extracted 2-3 times. During the process, the product started to precipitate. The mixture was washed with saturated sodium bicarbonate solution and water. Then the precipitated product was filtered off and dried to give the title compound as a white solid (55 mg).

MS (ESP): 476 (M+1) for C₂₀H₁₆F₃N₇O₂S

¹H-NMR (DMSO-d₆) δ: 1.10 (t, 3H); 2.31 (s, 3H); 3.05-3.28 (m, 2H); 7.74 (brs, 1H); 8.24 (s, 1H); 8.40 (s, 2H); 8.56 (s, 1H); 8.77 (d, 1H); 9.25 (d, 1H); 9.60 (s, 1H).

### Example 9

### 1-ethyl-3-(5-(5-(5-oxo-4,5-dihydro-1,3,4-oxadiazol-2-yl)thiazol-2-yl)-4-(4-(trifluoromethyl)thiazol-2-yl)pyridin-2-yl)urea

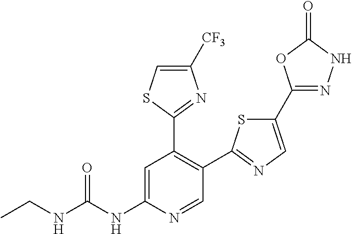

Methyl 2-(6-(3-ethylureido)-4-(4-(trifluoromethyl)thiazol-2-yl)pyridin-3-yl)thiazole-5-carboxylate (Intermediate 13; 128 mg, 0.28 mmol) was suspended hydrazine hydrate (0.2 mL, 6.43 mmol) and ethanol (5 mL). The slurry was heated until it became homogeneous. The reaction was monitored by LC/MS, and once it was determined to be complete, the reaction mixture concentrated to dryness. The solids were suspended in THF (5 mL) and diisopropylethylamine (0.073 mL, 0.42 mmol) and di(1H-imidazol-1-yl)methanone (45.4 mg, 0.28 mmol) were added. The mixture was heated to reflux, and the product precipitated out of solution. The solids were filtered and washed with methanol, then dried in vacuo. Isolation gave 24 mg of the title compound.

MS (ESP): 484 (M+1) for C₁₇H₁₂F₃N₇O₃S₂.

¹H NMR (300 MHz, d₆-DMSO): 1.03 (t, 3H), 3.14 (m, 2H), 7.42 (t, 1H), 8.03 (s, 1H), 8.30 (s, 1H), 8.66 (s, 1H), 8.68 (s, 1H), 9.63 (s, 1H), 12.73 (s, 1H).

### Example 10

### N-ethyl-N′-[5′-(5-oxo-4,5-dihydro-1,3,4-oxadiazol-2-yl)-4-(4-pyridin-2-yl-1,3-thiazol-2-yl)-3,3′-bipyridin-6-yl]urea

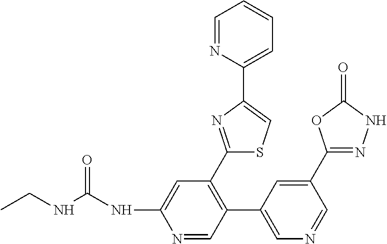

1,1′-Carbonylbis-1H-imidazole (0.050 g, 0.31 mmol) and DIEA (0.053 mL, 0.31 mmol) were added to a suspension of N-ethyl-N′45′-(hydrazinocarbonyl)-4-(4-pyridin-2-yl-1,3-thiazol-2-yl)-3,3′-bipyridin-6-yl]urea (Intermediate 22, 94 mg, 0.31 mmol) in DMF (2 mL), and the mixture was stirred at room temperature for 4 hours. The reaction mixture was concentrated under reduced pressure, then purified by Gilson HPLC (5-95% ACN/0.1% TFA in 14 min). Isolation gave 19 mg of the title compound.

LC/MS (ES⁺)[(M+H)⁺]: 487 for C₂₃H₁₈N₈O₃S.

¹H NMR (300 MHz, d₆-DMSO): 1.11 (t, 3H), 3.22 (m, 2H), 7.36 (t, 1H), 7.62 (t, 1H), 7.67 (d, 1H), 7.84 (m, 1H), 8.21 (t, 1H), 8.28 (s, 1H), 8.34 (s, 1H), 8.37 (s, 1H), 8.6 (d, 1H), 8.70 (d, 1H), 8.98 (d, 1H), 9.39 (s, 1H), 12.79 (s, 1H).

### Examples 11-12

The following compounds have been synthesized as described for Example 10 from the starting materials indicated in the table below.

| Ex | Compound | Data | SM |
|---|---|---|---|
| 11 | 1-ethyl-3-(5′-(5-oxo- 4,5-dihydro-1,3,4- oxadiazol-2-yl)-4-(4- phenylthiazol-2-yl)-3,3′- bipyridin-6-yl)urea 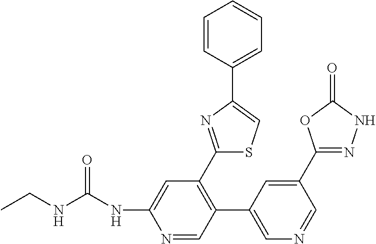 | LC/MS (ES⁺)[(M + H)⁺]: 486 for C₂₄H₁₉N₇O₃S. ¹H NMR (300 MHz, d₆-DMSO): 1.10 (t, 3H), 3.20 (m, 2H), 7.35 (m, 3H), 7.63 (t, 1H), 7.71 (d, 1H), 7.73 (d, 1H), 8.20 (t, 1H), 8.23 (s, 1H), 8.25 (s, 1H), 8.32 (s, 1H), 8.69 (d, 1H), 8.99 (d, 1H), 9.48 (s, 1H), 12.80 (s, 1H). | Intermediate 23 and CDI |
| 12 | 1-(4-(benzo[d]thiazol-2- yl)-5′-(5-oxo-4,5- dihydro-1,3,4- oxadiazol-2-yl)-3,3′- bipyridin-6-yl)-3- ethylurea 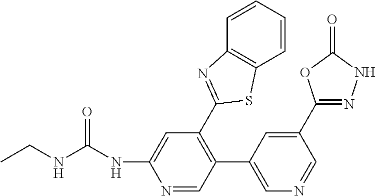 | LC/MS (ES⁺)[(M + H)⁺]: 460 for C₂₂H₁₇N₇O₃S. ¹H NMR (300 MHz, d₆-DMSO): 1.10 (t, 3H), 3.21 (m, 2H), 7.46-7.58 (m, 3H), 7.97 (d, 1H), 8.09 (d, 1H), 8.19 (t, 1H), 8.28 (s, 1H), 8.39 (s, 1H), 8.6 (d, 1H), 8.96 (d, 1H), 9.54 (s, 1H), 12.78 (s, 1H). | Intermediate 24 and CDI |

### Example 13

### 1-ethyl-3-(5-(quinoxalin-6-yl)-4-(4-trifluoromethyl)thiazol-2-yl)pyrimidin-2-yl)urea

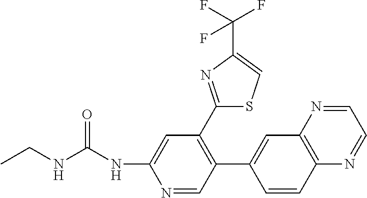

A reaction mixture of 1-ethyl-3-(5-(4,4,5,5-tetramethyl-1,3,2-dioxaborolan-2-yl)-4-(4-(trifluoromethyl)thiazol-2-yl)pyridin-2-yl)urea (Intermediate 12, 100 mg, 0.23 mmol), 6-bromoquinoxaline (43.0 mg, 0.21 mmol), Tetrakis (23.75 mg, 0.02 mmol), and cesium carbonate (73.7 mg, 0.23 mmol) in dioxane and water was prepared. The reaction mixture was degassed with nitrogen for 15 minutes and then heated to 100° C. for 1 h. The reaction mixture was partitioned between methylene chloride and water. The organic layer was washed with saturated sodium chloride, dried over sodium sulfate, filtered, and concentrated under reduced pressure. Purification by flash column chromatography (silica, 15:1 methylene chloride/methanol) gave 44 mg of desired product.

MS (ESP): 445 (M+1) for C₂₀H₁₅F₃N₆OS.

¹H NMR (300 MHz, DMSO-d₆): 1.12 (t, J=7 Hz, 3H), 3.24 (m, 2H), 7.23 (m, 1H), 7.43 (m, 1H), 8.04 (m, 1H), 8.21 (m, 1H), 8.36 (m, 1H), 8.55 (m, 1H), 9.02 (br s, 2H), 9.36 (s, 1H), 9.52 (s, 1H).

### Example 14

### 1-Ethyl-3-(4-(4-(trifluoromethyl)thiazol-2-yl)-3,3′-bipyridin-6-yl)urea

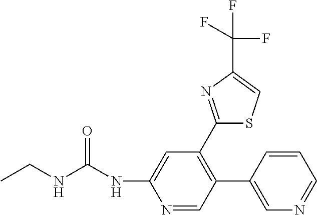

1-(5-Bromo-4-(4-(trifluoromethyl)thiazol-2-yl)pyridin-2-yl)-3-ethylurea (Intermediate 3, 70 mg, 0.18 mmol), 3-(4,4,5,5-tetramethyl-1,3,2-dioxaborolan-2-yl)pyridine, cesium carbonate (115 mg, 0.35 mmol), tetrakis(triphenylphosphine)palladium (0) (20.47 mg, 0.02 mmol) were taken in a microwave vial and degassed with nitrogen. Then dioxane:water (4:1, 5 mL) was added and the resulting mixture was heated in a microwave at 100° C. for 30 minutes. The palladium catalyst was filtered off and the filtrate was partitioned between water and ethyl acetate. The layers were separated and the aqueous layer was back extracted with ethyl acetate three times. The combined organic extract was washed with water and brine, dried over magnesium sulfate and concentrated under reduced pressure. The crude residue was washed with acetonitrile several times to give off-white solid (42 mg).

MS (ESP): 394 (M+1) for C₁₇H₁₄F₃N₅OS

¹H-NMR (DMSO-d₆) δ: 1.09 (t, 3H); 3.17-3.22 (m, 2H); 7.44-7.50 (m, 1H); 7.55 (t, 1H); 7.76 (d, 1H); 8.20 (s, 1H); 8.30 (s, 1H); 8.49 (s, 1H); 8.55 (s, 1H); 8.64 (d, 1H); 9.45 (s, 1H).

### Example 15

### 6′-(3-Ethylureido)-4′-(4-(trifluoromethyl)thiazol-2-yl)-3,3′-bipyridine 1-oxide

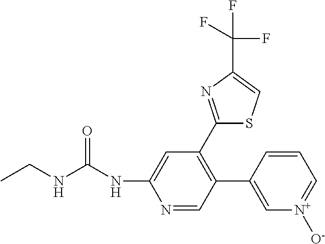

1-Ethyl-3-(5-(4,4,5,5-tetramethyl-1,3,2-dioxaborolan-2-yl)-4-(4-(trifluoromethyl)thiazol-2-yl)pyridin-2-yl)urea (Intermediate 12, 150 mg, 0.34 mmol), 3-bromopyridine 1-oxide, tetrakis(triphenylphosphine)palladium (0) (39.2 mg, 0.03 mmol), cesium carbonate (221 mg, 0.68 mmol) were taken in a microwave vial and degassed with nitrogen. Then dioxane:water (4:1, 5 mL) was added and the resulting mixture was heated in a microwave at 100° C. for 30 minutes. The product precipitated from the reaction as a white solid and was collected by filtration and washed with water and 1% methanol in dichloromethane to provide the desired product (27 mg).

MS (ESP): 410 (M+1) for C₁₇H₁₄F₃N₅O₂S

¹H-NMR (DMSO-d₆) δ: 1.09 (t, 3H); 3.12-3.22 (m, 2H); 7.23 (d, 1H); 7.40-7.45 (m, 1H); 7.45 (brs, 1H); 8.19 (s, 1H); 8.27 (d, 1H); 8.29 (s, 1H); 8.31 (s, 1H); 8.61 (s, 1H); 9.48 (s, 1H).

### Example 16

### 1-{5-(4,7-dioxo-4,5,6,7-tetrahydro[1,3]thiazolo[4,5-d]pyridazin-2-yl)-4-[4-(trifluoromethyl)-1,3-thiazol-2-yl]pyridin-2-yl}-3-ethylurea

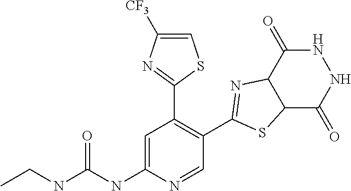

A solution of diethyl 2-(6-(3-ethylureido)-4-(4-(trifluoromethyl)thiazol-2-yl)pyridin-3-yl)thiazole-4,5-dicarboxylate (Intermediate 25, 150 mg, 0.28 mmol) and hydrazine hydrate (0.4 mL, 1.0N in MeOH) in methanol (10 mL) was refluxed for 5 h. The mixture was cooled and additional 0.4 mL of hydrazine hydrate-MeOH solution was added. The mixture was stirred for additional 5 h. The reaction mixture was cooled and 1.0 N HCl (1 mL) was added. The mixture was stirred at 45° C. for 1 h, cooled to room temperature, neutralized with powdered NaHCO₃, then purified via a reverse phase C18 column (10%-75% MeOH-water) to afford 70 mg (53%) of desired product as off-white powder.

MS (ESP): 484.0 (M+H⁺) for C₁₇H₁₂F₃N₇O₃S₂

¹H NMR (DMSO-d₆): δ ppm 1.11 (t, 3H), 3.21 (m, 2H), 7.46 (t, 1H), 8.16 (s, 1H), 8.72 (d, 1H), 8.78 (s, 1H), 9.78 (s, 1H)

### Example 17

### 1-Ethyl-3-(5′-(5-oxo-2,5-dihydro-1H-pyrazol-3-yl)-3,3′-bipyridin-6-yl)urea

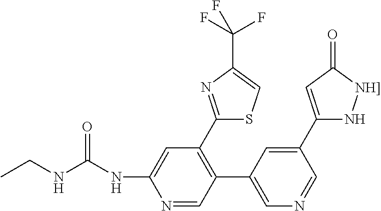

1-Ethyl-3-(5-(4,4,5,5-tetramethyl-1,3,2-dioxaborolan-2-yl)-4-(4-(trifluoromethyl)thiazol-2-yl)pyridin-2-yl)urea (Intermediate 12, 140 mg, 0.32 mmol), 5-(5-bromopyridin-3-yl)-1H-pyrazol-3(2H)-one (Intermediate 26, 76 mg, 0.32 mmol), cesium carbonate (103 mg, 0.32 mmol), tetrakis(triphenylphosphine)palladium(0) (36.6 mg, 0.03 mmol) and water (1.500 mL) were taken in a microwave vial and degassed with nitrogen. Then dioxane:water (8 mL 4:1) was added and the reaction mixture was heated in a microwave at 100° C. for 2 h. The reaction mixture was diluted with water and extracted with 5% MeOH in dichloromethane. The combined extract was dried over magnesium sulfate and concentrated under reduced pressure. The crude product was purified by reverse phase HPLC (25% to 70% ACN in water, 0.01% TFA). The fractions containing the product were combined, concentrated under reduced pressure and lypholized to give a white solid (42 mg) that was triturated with acetonitrile and dried under high vacuum.

MS (ESP): 476 (M+1) for C₂₀H₁₆F₃N₇O₂S

1H-NMR (DMSO-d₆) δ: 1.11 (t, 3H); 3.16-3.24 (m, 2H); 6.05 (s, 1H); 7.58 (brs, 1H); 8.18 (s, 1H); 8.28 (s, 1H); 8.38 (s, 1H); 8.45 (s, 1H); 8.56 (s, 1H); 9.01 (s, 1H); 9.51 (s, 1H).

### Example 18

### 1-Ethyl-3-(5′-(5-thioxo-4,5-dihydro-1,2,4-oxadiazol-3-yl)-4-(4-(trifluoromethyl)thiazol-2-yl)-3,3′-bipyridin-6-yl)urea

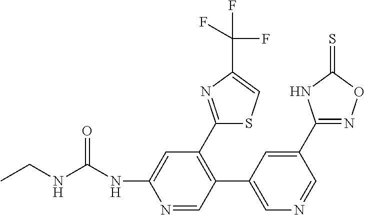

DBU (0.080 mL, 0.53 mmol) followed by di(1H-imidazol-2-yl)methanethione (35.5 mg, 0.20 mmol) were added to a mixture of 6′-(3-ethylureido)-N-hydroxy-4′-(4-(trifluoromethyl)thiazol-2-yl)-3,3′-bipyridine-5-carboximidamide (Intermediate 27, 60 mg, 0.13 mmol) in acetonitrile (3 mL), and the mixture was stirred at room temperature overnight. The reaction was concentrated and the residue was partitioned between water and ethyl acetate. The layers were separated and the organic layer was washed with water and brine, then dried over magnesium sulfate and concentrated under reduced pressure. The residue was purified by reverse phase chromatography and the fractions containing the product were combined and lyophilized to give white solid (22 mg, low yield).

MS (ESP): 494 (M+1) for C₁₉H₁₄F₃N₇O₂S₂

¹H-NMR (DMSO-d₆) δ: 1.10 (t, 3H); 3.16-3.24 (m, 2H); 7.56 (brs, 1H); 8.23 (s, 1H); 8.24 (s, 1H); 8.37 (s, 1H); 8.58 (s, 1H); 8.70 (d, 1H); 9.06 (s, 1H); 9.52 (s, 1H).

### Example 19

### 1-Ethyl-3-(5′-(5-oxo-4,5-dihydro-1,2,4-oxadiazol-3-yl)-4-(4-(trifluoromethyl)thiazol-2-yl)-3,3′-bipyridin-6-yl)urea

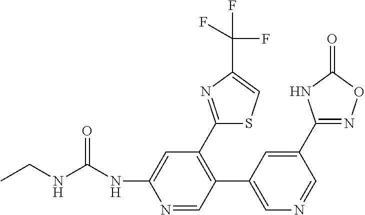

DBU (0.023 mL, 0.16 mmol) followed by carbonyl diimidazole (25.1 mg, 0.16 mmol) were added to a suspension of 6′-(3-ethylureido)-N′-hydroxy-4′-(4-(trifluoromethyl)thiazol-2-yl)-3,3′-bipyridine-5-carboximidamide (Intermediate 27, 70 mg, 0.16 mmol) in dioxane (3 mL). The resulting solution was stirred overnight at room temperature. The solvent was removed and the crude residue was partitioned between water and ethyl acetate. The layers were separated and the aqueous was back extracted with ethyl acetate three times. The aqueous layer was concentrated under reduced pressure and purified by reverse phase HPLC (5% ACN in water to 70% ACN) to give the title compound as a white solid (33 mg).

MS (ESP): 478 (M+1) for C₁₉H₁₄F₃N₇O₃S

¹H-NMR (DMSO-d₆) δ: 1.09 (t, 3H); 3.15-3.24 (m, 2H); 7.51 (br s, 1H); 8.15 (s, 1H); 8.24 (s, 1H); 8.37 (s, 1H); 8.59 (s, 1H); 8.70 (s, 1H); 8.99 (d, 1H); 9.52 (s, 1H); 13.14 (br s, 1H).

### Example 20

### N-Ethyl-N′-{5′-(2-Oxido-3H-1,2,3,5-oxathiadiazol-4-yl)-4-[4-trifluoromethyl)-1,3-thiazol-2-yl]-3,3′-bipyridine-6-yl}urea

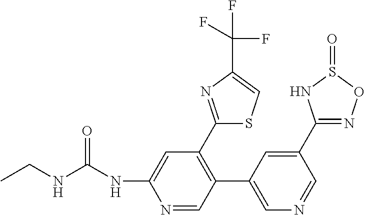

To a suspension of 6′-(3-ethylureido)-N-hydroxy-4′-(4-(trifluoromethyl)thiazol-2-yl)-3,3′-bipyridine-5-carboximidamide (Intermediate 27, 70 mg, 0.16 mmol) in THF (1.5 mL) at 0° C., pyridine (0.025 mL, 0.31 mmol) was added followed by a drop wise addition of a solution of sulfurous dichloride (0.023 mL, 0.31 mmol) in dichloromethane (1.5 mL). The resulting mixture was slowly warmed up to room temperature and allowed to stir for an hour. Then the reaction was quenched by adding water (1 mL). The layers were separated and the aqueous layer was back extracted with 1% MeOH in DCM twice and the combined organic layers were washed with water and brine, then dried over magnesium sulfate, filtered and concentrated under reduced pressure. The resulting residue was purified by normal phase chromatography to give the title compound as a white solid (25 mg).

MS (ESP): 498 (M+1) for C₁₈H₁₄F₃N₇O₃S₂

¹H-NMR (DMSO-d₆) δ: 1.10 (t, 3H); 3.18-3.22 (m, 2H); 7.58 (br s, 1H); 8.17 (t, 1H); 8.25 (s, 1H); 8.36 (s, 1H); 8.54 (d, 1H); 8.57 (d, 1H); 9.04 (d, 1H); 9.50 (br s, 1H).

### Example 21

### 1-(5′-Bromo-4-(4-(trifluoromethyl)thiazol-2-yl)-3,3′-bipyridin-6-yl)-3-ethylurea

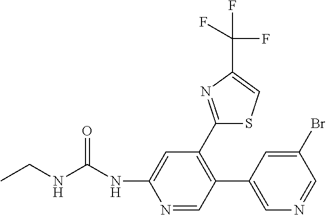

3-Bromo-5-(4,4,5,5-tetramethyl-1,3,2-dioxaborolan-2-yl)pyridine (596 mg, 2.10 mmol), 1-(5-bromo-4-(4-(trifluoromethyl)thiazol-2-yl)pyridin-2-yl)-3-ethylurea (Intermediate 3, 830 mg, 2.10 mmol), tris(dibenzylideneacetone)dipalladium(0) (192 mg, 0.21 mmol), 2-dicyclohexylphosphino-2′,4′,6′-tri-iso-propyl-1,1′-biphenyl (300 mg, 0.63 mmol) and sodium carbonate (223 mg, 2.10 mmol) were taken in a round bottomed flask, and the flask was flushed with nitrogen. Solvent (5:1; acetonitrile, water, 10 mL) was added and degassed with nitrogen, and the mixture was heated at 100° C. for 3 h. The reaction mixture was filtered and the filtrate was concentrated under reduced pressure. The resulting crude residue was partitioned between water and ethyl acetate. The layers were separated and the aqueous was back extracted with ethyl acetate three times. The combined organic layers were washed with water and brine, dried over magnesium sulfate and concentrated under reduced pressure. The residue obtained was purified by normal phase chromatography (gradient of MeOH in DCM) to give a white solid (483 mg).

MS (ESP): 473 (M+1) for C₁₇H₁₃BrF₃N₅OS

### Example 22-24

The following Examples were synthesized according to the procedure described for Intermediate 2 from the starting materials indicated.

| Ex | Compound | Data | SM |
|---|---|---|---|
| 22 | 1-Ethyl-3-(5′- (methylsulfonyl)-4-(4- (trifluoromethyl)thiazol- 2-yl)-3,3′-bipyridin-6- yl)urea 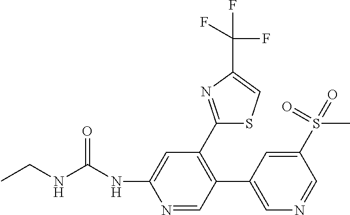 | MS (ESP): 472 (M + 1) for C₁₈H₁₆F₃N₅O₃S₂ ¹H-NMR (DMSO-d₆) δ: 1.11 (t, 3H); 3.16-3.28 (m, 2H); 3.31 (s, 3H); 7.53 (brs, 1H); 8.23 (s, 1H); 8.25 (t, 1H); 8.42 (s, 1H); 8.60 (d, 1H); 8.84 (d, 1H); 9.08 (d, 1H); 9.54 (brs, 1H). | Intermediate 3 and 5- (methylsulfonyl)pyridin- 3-ylboronic acid |
| 23 | 6′-(3-Ethylureido)-4′-(4- (trifluoromethyl)thiazol- 2-yl)-3,3′-bipyridine-5- sulfonamide 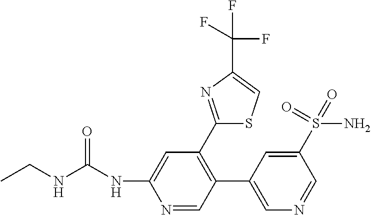 | MS (ESP): 473 (M + 1) for C₁₇H₁₅F₃N₆O₃S₂ ¹H-NMR (DMSO-d₆) δ: 1.11 (t, 3H); 3.17-3.28 (m, 2H); 7.43 (brs, 1H); 7.63 (s, 2H); 8.10 (d, 1H); 8.25 (s, 1H); 8.36 (s, 1H); 8.60 (s, 1H); 8.72 (d, 1H); 8.98 (d, 1H); 9.53 (s, 1H). | Intermediate 12 and 5- bromopyridine-3- sulfonamide |
| 24 | 1-Ethyl-3-(5′-(1-methyl- 1H-pyrazol-4-yl)-4-(4- (trifluoromethyl)thiazol- 2-yl)-3,3′-bipyridin-6- yl)urea 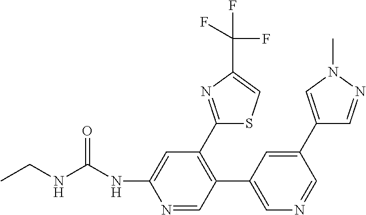 | MS (ESP): 474 (M + 1) for C₂₁H₁₈F₃N₇OS ¹H-NMR (DMSO-d₆) δ: 1.11 (t, 3H); 3.14-3.25 (m, 2H); 3.87 (s, 3H); 7.59 (brs, 1H); 7.98 (s, 1H); 8.02 (s, 1H); 8.28 (d, 3H); 8.35 (s, 1H); 8.54 (s, 1H); 8.90 (d, 1H); 9.47 (s, 1H). | Example 21 and 1- methyl-4-(4,4,5,5- tetramethyl-1,3,2- dioxaborolan-2-yl)- 1H-pyrazole |

### Example 25

### 1-(5′-(1H-Imidazol-2-yl)-4-(4-(trifluoromethyl)thiazol-2-yl)-3,3′-bipyridin-6-yl)-3-ethylurea

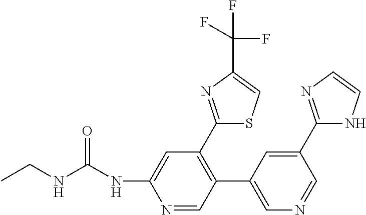

Sodium methoxide (10.291 μl, 0.06 mmol) was added to a suspension of 1-(5′-cyano-4-(4-(trifluoromethyl)thiazol-2-yl)-3,3′-bipyridin-6-yl)-3-ethylurea (Example 2, 120 mg, 0.29 mmol) in methanol (3 mL), and the resulting mixture was stirred overnight at room temperature. 2,2-Dimethoxyethanamine (30.9 μl, 0.29 mmol) followed by acetic acid (32.8 μl, 0.57 mmol) were added, and the mixture was heated to 50° C. for 1.5 hours. The reaction mixture was cooled to room temperature and isopropanol (3 mL) followed by HCl (500 μl, 6N) were added and the mixture was refluxed overnight. The solvent was removed and the residue was dissolved in water and neutralized by adding 2N NaOH. The aqueous layer was extracted with ethyl acetate, and the ethyl acetate layer was washed with water and brine, dried over magnesium sulfate, and concentrated under reduced pressure. The off-white solid obtained was triturated with acetonitrile and dried to give a white solid (43 mg).

MS (ESP): 460 (M+1) for C₂₀H₁₆F₃N₇OS

¹H-NMR (DMSO-d₆) δ: 1.11 (t, 3H); 3.14-3.25 (m, 2H); 7.09 (s, 1H); 7.35 (s, 1H); 7.58 (brs, 1H); 8.24 (s, 1H); 8.28 (s, 1H); 8.37 (s, 1H); 8.43 (s, 1H); 8.53 (s, 1H); 9.19 (s, 1H); 9.49 (s, 1H); 12.73 (s, 1H).

### Example 26

### 1-(5′-(4,5-Dihydrooxazol-2-yl)-4-(4-(trifluoromethyl)thiazol-2-yl)-3,3′-bipyridin-6-yl)-3-ethylurea

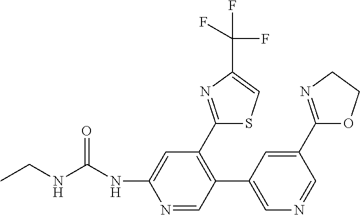

Bismuth(III) trifluoromethanesulfonate (10.98 mg, 0.02 mmol) was added to a suspension of 1-(5′-cyano-4-(4-(trifluoromethyl)thiazol-2-yl)-3,3′-bipyridin-6-yl)-3-ethylurea (Example 2, 100 mg, 0.24 mmol) and 2-aminoethanol (115 μl, 1.91 mmol), and the resulting reaction mixture was stirred at 70° C. for overnight. The reaction mixture was partitioned between water and 3% MeOH in ethyl acetate. The layers were separated and the aqueous layer was back extracted twice with 3% methanol in ethyl acetate. The combined organic layers were dried over magnesium sulfate and concentrated under reduced pressure to give a white solid. The solid was triturated with acetonitrile and dried under high vacuum to give the product as a white solid (26 mg).

MS (ESP): 463 (M+1) for C₂₀H₁₇F₃N₆O₂S

¹H-NMR (DMSO-d₆) δ: 1.11 (t, 3H); 3.14-3.28 (m, 2H); 3.99 (t, 2H); 4.43 (t, 2H); 7.57 (t, 1H); 8.12 (t, 1H); 8.23 (s, 1H); 8.36 (s, 1H); 8.56 (s, 1H); 8.65 (d, 1H); 9.04 (s, 1H); 9.50 (s, 1H).

### Examples 27-28

The following Examples were synthesized according to the procedure described for Example 10 from the starting materials indicated.

| Ex | Compound | Data | SM |
|---|---|---|---|
| 27 | 1-ethyl-3-(4-(4-(1- methyl-1H-pyrazol-4- yl)thiazol-2-yl)-5′-(5- oxo-4,5-dihydro-1,3,4- oxadiazol-2-yl)-3,3′- bipyridin-6-yl)urea 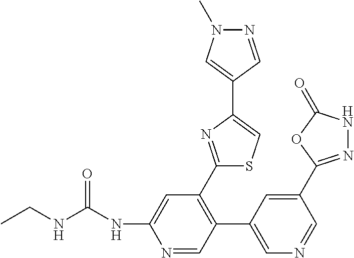 | LC/MS (ES⁺)[(M + H)⁺]: 490 for C₂₂H₁₉N₉O₃S. ¹H NMR (300 MHz, d₆-DMSO): 1.11 (t, 3H), 3.22 (m, 2H), 3.84 (s, 3H), 7.62 (s, 1H), 7.63 (m, 1H), 7.78 (s, 1H), 7.92 (s, 1H), 8.16 (m, 1H), 8.19 (s, 1H), 8.32 (s, 1H), 8.66 (d, 1H), 8.98 (d, 1H), 9.42 (s, 1H), 12.81 (s, 1H). | Intermediate 36 and 1,1′-carbonylbis-1H- imidazole, diispropylethyl amine |
| 28 | 1-ethyl-3-(4-(1-methyl- 1H-pyrazol-5-yl)-5′-(5- oxo-4,5-dihydro-1,3,4- oxadiazol-2-yl)-3,3′- bipyridin-6-yl)urea 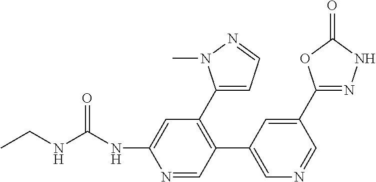 | LC/MS (ES⁺)[(M + H)⁺]: 407 for C₁₉H₁₈N₈O₃. ¹H NMR (300 MHz, d₆-DMSO): 1.06 (t, 3H), 3.16 (m, 2H), 3.78 (s, 3H), 7.03 (m, 1H), 7.40 (s, 1H), 7.86 (m, 1H), 7.92 (t, 1H), 8.07 (s, 1H), 8.32 (m, 1H), 8.53 (d, 1H), 8.77 (d, 1H), 9.44 (s, 1H), 12.76 (s, 1H). | Intermediate 35 and 1,1′-carbonylbis-1H- imidazole, diispropylethyl amine |

### Example 29

### 1-(6-1H-pyrazol-1-yl)-4′-(4-trifluoromethyl)thiazol-2-yl)-2,3′-bipyridin-6′-yl)-3-ethylurea

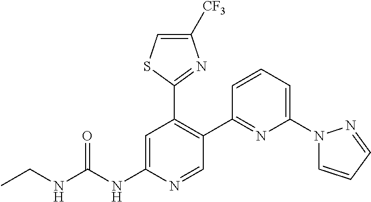

In a microwave reaction vessel, 1-(5-bromo-4-(4-(trifluoromethyl)thiazol-2-yl)pyridin-2-yl)-3-ethylurea (Intermediate 3, 200 mg, 0.51 mmol), 2-(1H-pyrazol-1-yl)-6-(4,4,5,5-tetramethyl-1,3,2-dioxaborolan-2-yl)pyridine (137 mg, 0.51 mmol) and cesium carbonate (64.4 mg, 0.61 mmol) were combined and suspended in a 4:1 mixture of dioxane and water. Pd(PPh₃)₄ (29.2 mg, 0.03 mmol) was added in a single portion. The vessel was sealed, degassed, purged with nitrogen and heated to 100° C. in the microwave for 120 min. The crude reaction mixture was concentrated to dryness. The resulting residue was dissolved in DMSO, filtered and then purified by Gilson HPLC (5-95% ACN/0.1% TFA water in 14 minutes). Isolation gave 56 mg of the title compound.

LC/MS (ES⁺)[(M+H)⁺]: 460 for C₂₀H₁₆F₃N₇OS.

¹H NMR (300 MHz, d₆-DMSO): 1.11 (t, 3H), 3.20 (m, 2H), 6.47 (m, 1H), 7.59 (d, 1H), 7.61 (m, 1H), 7.79 (m, 1H), 7.85 (d, 1H), 7.95 (d, 1H), 8.06 (m, 2H), 8.55 (m, 1H), 8.62 (s, 1H), 9.56 (s, 1H).

### Example 30

### 1-ethyl-3-(5-(2-morpholinothiazol-4-yl)-4-(4-(trifluoromethyl)thiazol-2-yl)pyridin-2-yl)urea

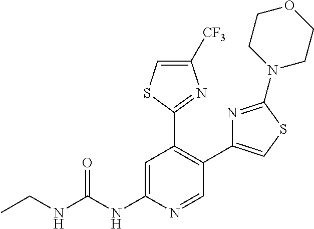

In a microwave reaction vessel, 1-(5-bromo-4-(4-(trifluoromethyl)thiazol-2-yl)pyridin-2-yl)-3-ethylurea (Intermediate 3, 100 mg, 0.25 mmol), 4-(4-(4,4,5,5-tetramethyl-1,3,2-dioxaborolan-2-yl)thiazol-2-yl)morpholine (82 mg's, 0.28 mmol), sodium carbonate (40 mg, 0.38 mmol), Pd₂(dba)₃ (23.17 mg, 0.03 mmol) and X-Phos (2-(Dicyclohexylphosphino)-2′,4′,6′-tri-1-propyl-1,1′-biphenyl) (38.1 mg, 0.08 mmol) were combined and suspended in 4:1 mixture of acetonitrile (3 mL) and water (0.75 mL). The vessel was sealed and heated to 90° C. in an oil bath for 30 min. The reaction mixture was cooled to room temperature and concentrated to dryness. The crude residue was dissolved in minimal DMSO, filtered and then purified by Gilson HPLC (5-95% ACN/0.1% TFA water in 14 min.). Isolation gave 58 mg of the compound.

LC/MS (ES⁺)[(M+H)⁺]: 485 for C₁₉H₁₉F₃N₆O₂S₂.

¹H NMR (300 MHz, d₆-DMSO): 1.03 (t, 3H), 3.11 (m, 2H), 3.18 (m, 4H), 3.58 (m, 4H), 7.01 (s, 1H), 7.54 (t, 1H), 8.00 (s, 1H), 8.35 (s, 1H), 8.56 (s, 1H), 9.31 (s, 1H).

### Examples 31-32

The following Examples were synthesized according to the procedure for Example 30 from the starting materials indicated below.

| Ex | Compound | Data | SM |
|---|---|---|---|
| 31 | 1-(5-(6-cyanopyrazin-2- yl)-4-(4- (trifluoromethyl)thiazol- 2-yl)pyridin-2-yl)-3- ethylurea 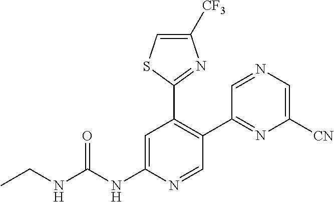 | LC/MS (ES⁺)[(M + H)⁺]: 420 for C₁₇H₁₂F₃N₇OS. ¹H NMR (300 MHz, d₆-DMSO): 1.11 (t, 3H), 3.20 (m, 2H), 7.50 (m, 1H), 8.18 (s, 1H), 8.61 (s, 1H), 8.68 (s, 1H), 9.08 (s, 1H), 9.20 (s, 1H), 9.67 (s, 1H). | Intermediate 3 and 6- (4,4,5,5-tetramethyl- 1,3,2-dioxaborolan-2- yl)pyrazine-2- carbonitrile |
| 32 | 1-(6-cyano-4′-(4- (trifluoromethyl)thiazol- 2-yl)-2,3′-bipyridin-6′- yl)-3-ethylurea 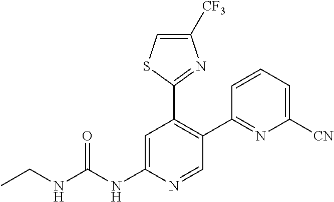 | LC/MS (ES⁺)[(M + H)⁺]: 418 for C₁₈H₁₂F₃N₆OS. ¹H NMR (300 MHz, d₆-DMSO): 1.04 (t, 3H), 3.12 (m, 2H), 7.49 (m, 1H), 7.80 (m, 1H), 7.93 (m, 1H), 8.01 (m, 1H), 8.04 (s, 1H), 8.45 (s, 1H), 8.56 (s, 1H), 9.51 (s, 1H). | Intermediate 3 and 6- (4,4,5,5-tetramethyl- 1,3,2-dioxaborolan-2- yl)picolinonitrile |

### Example 33

### 1-ethyl-3-(2′-(5-oxo-4,5-dihydro-1,3,4-oxadiazol-2-yl)-4-(4-(trifluoromethyl)thiazol-2-yl)-3,4′-bipyridin-6-yl)urea

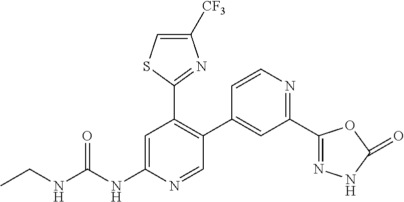

6-(3-Ethylureido)-4-(4-(trifluoromethyl)thiazol-2-yl)-3,4′-bipyridine-2′-carboxylic acid (Intermediate 50, 72.1 mg, 0.16 mmol) was dissolved in a DMF solution containing diisopropylethyl amine (0.057 mL, 0.33 mmol) and HATU (75 mg, 0.20 mmol). The solution was allowed to stir for 30 min., then hydrazine monohydrate (0.052 mL, 1.65 mmol) was added in a single in portion. The reaction mixture was diluted with EtOAc then washed with water. The organic layer was dried over Na₂SO₄, filtered and concentrated under reduced pressure.

The crude reaction mixture was dissolved in THF (2 mL) and carbonyl diimidazole (66 mg, 0.41 mmol) was added in a single portion. The reaction mixture was heated to reflux in a sealed microwave vial. The crude reaction mixture was concentrated under reduced pressure. The resulting residue was treated with water and the solid that formed was collected by filtration, washed with water and dried in vacuo. Isolation gave 61 mg of the crude product. The crude product was dissolved in minimal DMSO and purified by Gilson HPLC (5-95% ACN/0.1% TFA water in 14 min). Isolation gave 21 mg of the title compound.

LC/MS (ES⁺)[(M+H)⁺]: 478 for C₁₉H₁₄F₃N₇O₃S.

¹H NMR (300 MHz, d₆-DMSO): 1.04 (t, 3H), 3.12 (m, 2H), 7.43 (d, 1H), 7.46 (t, 1H), 7.73 (s, 1H), 8.10 (s, 1H), 8.32 (s, 1H), 8.55 (s, 1H), 8.62 (d, 1H), 9.49 (s, 1H), 12.74 (s, 1H).

### Example 34-39

The following Examples were prepared according to the procedure described for Example 9 from the indicated starting materials.

| Ex | Compound | Data | SM |
|---|---|---|---|
| 34 | 1-ethyl-3-(5-(4-methyl-6- (5-oxo-4,5-dihydro-1,3,4- oxadiazol-2-yl)pyrimidin- 2-yl)-4-(4- (trifluoromethyl)thiazol- 2-yl)pyridin-2-yl)urea | LC/MS (ES⁺)[(M + H)⁺]: 493 for C₁₉H₁₅F₃N₈O₃S. ¹H NMR (300 MHz, d₆-DMSO): 1.04 (t, 3H), 2.35 (s, 3H), 3.12 (m, 2H), 7.54 (t, 1H), 7.68 (s, 1H), 7.92 (s, 1H), 8.55 (s, 1H), 8.79 (s, 1H), 9.62 (s, 1H), 12.99 (s, 1H). | Intermediate 41 and CDI |
| | 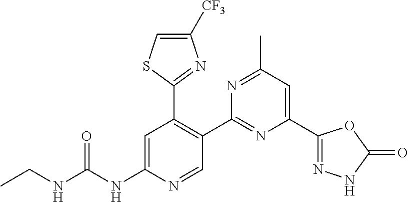 | | |
| 35 | 1-ethyl-3-(5-(6-(5-oxo- 4,5-dihydro-1,3,4- oxadiazol-2-yl)pyrazin- 2-yl)-4-(4- (trifluoromethyl)thiazol- 2-yl)pyridin-2-yl)urea | LC/MS (ES⁺)[(M + H)⁺]: 479 for C₁₈H₁₃F₃N₈O₃S. ¹H NMR (300 MHz, d₆-DMSO): 1.10 (t, 3H), 3.19 (m, 2H), 7.53 (t, 1H), 8.15 (s, 1H), 8.60 (s, 1H), 8.61 (s, 1H), 8.88 (s, 1H), 9.06 (s, 1H), 9.63 (s, 1H), 12.97 (s, 1H). | Intermediate 42 and CDI |
| | 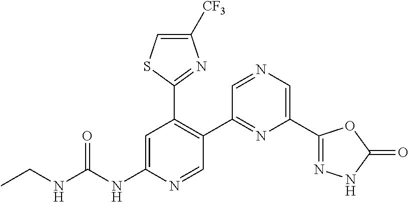 | | |
| 36 | 1-ethyl-3-(5-(5-(5-oxo- 4,5-dihydro-1,3,4- oxadiazol-2-yl)-4- (pyrimidin-2-yl)thiazol- 2-yl)-4-(4- (trifluoromethyl)thiazol- 2-yl)pyridin-2-yl)urea | LC/MS (ES⁺)[(M + H)⁺]: 562 for C₂₁H₁₄F₃N₉O₃S₂. ¹H NMR (300 MHz, d₆-DMSO): 1.09 (t, 3H), 3.18 (m, 2H), 7.47 (t, 1H), 7.57 (t, 1H), 8.15 (s, 1H), 8.72 (s, 1H), 8.79 (s, 1H), 8.91 (d, 2H), 9.72 (s, 1H), 12.08 (s, 1H). | Intermediate 39 and CDI |
| | 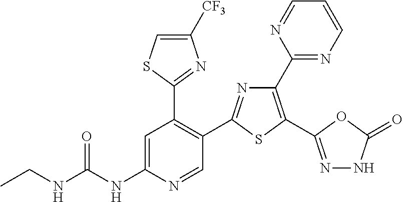 | | |
| 37 | 1-ethyl-3-(5-(4-(1- mcthyl-1H-1,2,4- triazol-5-yl)-5-(5-oxo- 4,5-dihydro-1,3,4- oxadiazol-2-yl)thiazol- 2-yl)-4-(4- (trifluoromethyl)thiazol- 2-yl)pyridin-2-yl)urea | LC/MS (ES⁺)[(M + H)⁺]: 565 for C₂₀H₁₅F₃N₁₀O₃S₂. ¹H NMR (300 MHz, d₆-DMSO): 1.09 (t, 3H), 3.17 (m, 2H), 3.77 (s, 3H), 7.46 (t, 1H), 8.07 (s, 1H), 8.09 (s, 1H), 8.73 (s, 1H), 8.82 (s, 1H), 9.74 (s, 1H), 12.88 (s, 1H). | Intermediate 40 and CDI |
| | 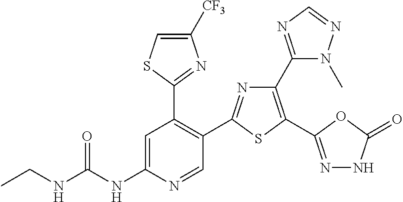 | | |
| 38 | 1-ethyl-3-(5′-(5-oxo-4,5- dihydro-1,3,4-oxadiazol- 2-yl)-4-(5-(pyridin-4-yl)- 1,3,4-oxadiazol-2-yl)- 3,3′-bipyridin-6-yl)urea: | LC/MS (ES⁺)[(M + H)⁺]: 472 for C₂₂H₁₇N₉O₄. ¹H NMR (300 MHz, d₆-DMSO): 1.11 (t, 3H), 3.22 (m, 2H), 7.44 (t, 1H), 7.68 (d, 2H), 8.28 (d, 1H), 8.44 (s, 1H), 8.51 (s, 1H), 8.80 (s, 1H), 8.81 (d, 2H), 9.03 (d, 1H), 9.59 (s, 1H), 12.77 (s, 1H). | Intermediate 54 and CDI |
| | 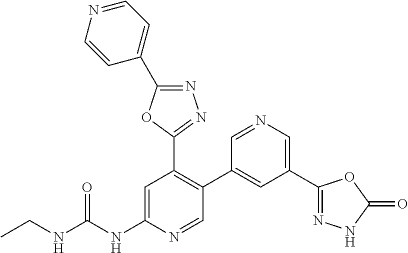 | | |
| 39 | N-ethyl-N′-[5′-(5-oxo- 4,5-dihydro-1,3,4- oxadiazol-2-yl)-4-(4- pyridin-4-yl-1,3-thiazol- 2-yl)-3,3′-bipyridin-6- yl]urea | LC/MS (ES⁺)[(M+H)⁺]: 487 for C₂₃H₁₈N₈O₃S. ¹H NMR (300 MHz, d₆-DMSO): 1.11 (t, 3H), 3.22 (m, 2H), 7.36 (t, 1H), 7.62 (t, 1H), 7.67 (d, 1H), 7.84 (m, 1H), 8.21 (t, 1H), 8.28 (s, 1H), 8.34 (s, 1H), 8.37 (s, 1H), 8.62 (d, 1H), 8.65 (d, 1H), 8.98 (d, 1H), 9.39 (s, 1H), 12.79 (s, 1H). | Intermediate 34 and CDI |
| | 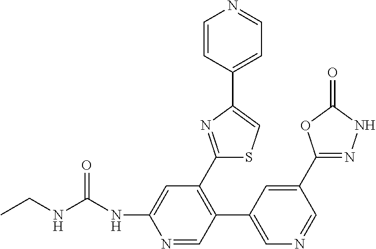 | | |

### Example 40

### 1-(6′-butoxy-5′-(5-oxo-4,5-dihydro-1,3,4-oxadiazol-2-yl)-4-(4-(trifluoromethyl)thiazol-2-yl)-3,3′-bipyridin-6-yl)-3-ethylurea

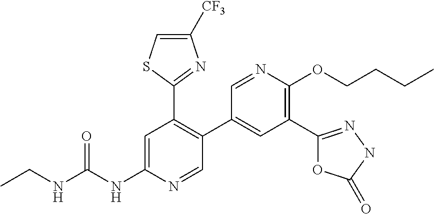

6-Butoxy-6′-(3-ethylureido)-4′-(4-(trifluoromethyl)thiazol-2-yl)-3,3′-bipyridine-5-carboxylic acid (Intermediate 57, <100 mg) was stirred in anhydrous MeOH in the presence of catalytic amount of SOCl₂ overnight at rt. The mixture was concentrated and the residue was dissolved in EtOH, and treated with >10 eq. of hydrazine hydrate at 70-80° C. for 48 h. The mixture was concentrated and residue was purified via a reverse phase column (10-60% EtOH-water). The hydrazide product was dissolved in THF, treated with 1.5 eq. of carbonyl diimidazole and Et₃N at room temperature for 1 h. The reaction mixture was concentrated and purified via a silica gel column chromatograph with heptane-EtOAc (1:1)+2% EtOH to give a 15% yield of 1-(6′-butoxy-5′-(5-oxo-4,5-dihydro-1,3,4-oxadiazol-2-yl)-4-(4-(trifluoromethyl)thiazol-2-yl)-3,3′-bipyridin-6-yl)-3-ethylurea.

MS (ESP): 550.2 (M+H⁺) for C₂₃H₂₂F₃N₇O₄S.

¹H NMR (CD₃OD): δ ppm 0.99 (t, 3H), 1.22 (t, 3H), 1.43-1.59 (m, 2H), 1.75-1.83 (m, 2H), 3.31 (q, 2H), 4.49 (t, 2H), 7.87 (s, 1H), 7.99 (d, 1H), 8.16 (d, 1H), 8.21 (d, 1H), 8.32 (d, 1H)

### Example 41

The following Example was prepared according to the procedure for Example 1 from the starting materials indicated.

| Ex | Compound | Data | SM |
|---|---|---|---|
| 41 | 6-Butoxy-1-ethyl-3-(5′- (5-methyl-1,3,4- oxadiazol-2-yl)-4-(4- (trifluoromethyl)thiazol- 2-yl)-3,3′-bipyridin-6- yl)urea | ¹H NMR (CD₃OD): δ ppm 0.99 (t, 3H), 1.22 (t, 3H), 1.43-1.59 (m, 2 H), 1.75-1.83 (m, 2H), 2.61 (s, 3H), 3.31 (q, 2H), 4.49 (t, 2H), 7.87 (s, 1H), 7.99 (d, 1 H), 8.16 (d, 1H), 8.21 (d, 1H), 8.32 (d, 1H) | Intermediate 57 and acetohydrazide |
| | 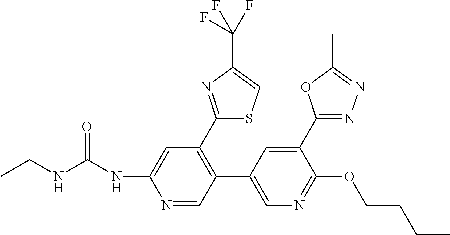 | | |

### Example 42

### 1-isopropyl-3-(5′-(5-oxo-4,5-dihydro-1,3,4-oxadiazol-2-yl)-4-(4-(trifluoromethyl)thiazol-2-yl)-3,3′-bipyridin-6-yl)urea

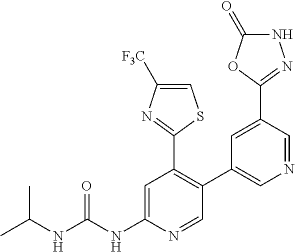

To a 100 mL of round bottom flask was charged methyl 6′-(3-isopropylureido)-4′-(4-(trifluoromethyl)thiazol-2-yl)-3,3′-bipyridine-5-carboxylate (Intermediate 79, 80 mg, 0.172 mmol) with ethanol (20 mL), then hydrazine monohydrate (3 mL) was added. The mixture was heated at reflux for 1.5 h. The mixture was concentrated under reduced pressure to give a white solid. To the crude material was charged anhydrous tetrahydrofuran (20 mL) with 1,1′-carbonyl diimidazole (1.43 g). The mixture was allowed to stir at room temperature overnight. The mixture was concentrated to dryness, water was added and the mixture was allowed to stand for 1-2 hours. A white solid precipitated from the water and was collected then dried in vacuo overnight at 50° C. to give a white solid (56 mg, 66.4%).

MS (ESP): 492.0 (MH⁺) for C₂₀H₁₆F₃N₇O₃S

¹H NMR (300 MHz, CD₃OD): δ 1.25 (d, 6H), 3.99 (m, 1H), 7.90 (s, 1H), 8.17 (t, 1H), 8.25 (d, 1H), 8.37 (d, 1H), 8.57 (d, 1H), 9.00 (d, 1H)

¹⁹F NMR (CD₃OD) −66.00

### Example 43-50

The following Examples were prepared as described for Example 42 from the starting materials indicated in the Table.

| Ex | Compound | Data | SM |
|---|---|---|---|
| 43 | 1-methyl-3-(5′-(5-oxo- 4,5-dihydro-1,3,4- oxadiazol-2-yl)-4-(4- (trifluoromethyl)thiazol- 2-yl)-3,3′-bipyridin-6- yl)urea | MS (ESP): 464.1 (MH⁺) for C₁₈H₁₂F₃N₇O₃S ¹H NMR (300 MHz, CD₃OD): δ 2.90 (s, 3H), 7.81 (s, 1H), 8.16 (t, 1H), 8.25 (d, 1H), 8.37 (d, 1H), 8.54 (d, 1H), 9.00 (d, 1H) ¹⁹F NMR (300 MHz, CD₃OD) −65.99 | Intermediate 70 |
| | 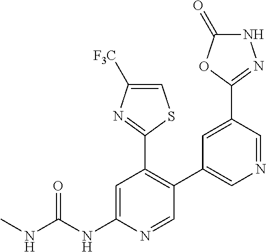 | | |
| 44 | 1-(5′-(5-oxo-4,5-dihydro- 1,3,4-oxadiazol-2-yl)-4- (4- (trifluoromethyl)thiazol- 2-yl)-3,3′-bipyridin-6-yl)- 3-propylurea | MS (ESP): 492.0 (MH⁺) for C₂₀H₁₉F₃N₇O₃S ¹H NMR (300 MHz, CD₃OD): δ 0.99 (t, 3H), 1.59-1.66 (m, 2H), 3.29 (t, 2H), 7.86 (s, 1H), 8.17 (t, 1H), 8.25 (d, 1H), 8.38 (d, 1H), 8.58 (d, 1H), 9.00 (d, 1H) ¹⁹F NMR (300 MHz, CD₃OD) −66.00 | Intermediate 80 |
| | 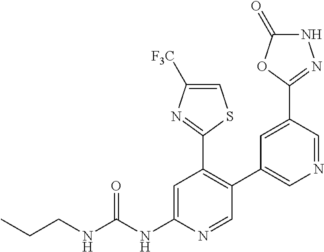 | | |
| 45 | 1-cyclopropyl-3-(5′-(5- oxo-4,5-dihydro-1,3,4- oxadiazol-2-yl)-4-(4- (trifluoromethyl)thiazol- 2-yl)-3,3′-bipyridin-6- yl)urea | MS (ESP): 490.2 (MH⁺) for C₂₀H₁₄F₃N₇O₃S ¹H NMR (300 MHz, CD₃OD): δ 0.56-0.61 (m, 2H), 0.76-0.82 (m, 2H), 2.67-2.72 (m, 1H), 7.96 (br, 1H), 8.14 (s, 1H), 8.24 (s, 1H), 8.36 (s, 1H), 8.50 (s, 1H), 8.99 (s, 1H) ¹⁹F NMR (300 MHz, CD₃OD) −65.97 | Intermediate 71 |
| | 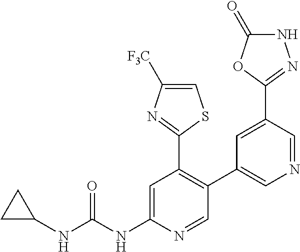 | | |
| 46 | 1-cyclohexyl-3-(5′-(5- oxo-4,5-dihydro-1,3,4- oxadiazol-2-yl)-4-(4- (trifluoromethyl)thiazol- 2-yl)-3,3′-bipyridin-6- yl)urea | MS (ESP): 532.2 (MH⁺) for C₂₃H₂₀F₃N₇O₃S ¹H NMR (300 MHz, CD₃OD): δ 1.28-1.46 (m, 5H), 1.60-1.99 (m, 5H), 3.70 (br, 1H), 7.89 (d, 1H), 8.17 (t, 1H), 8.26 (d, 1H), 8.38 (d, 1H), 8.58 (d, 1H), 9.00 (d, 1H) ¹⁹F NMR (300 MHz, CD₃OD) −66.00 | Intermediate 72 |
| | 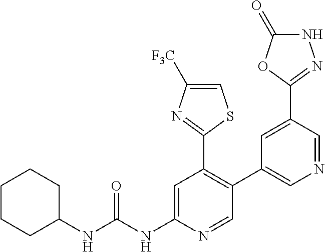 | | |
| 47 | 1,1-diethyl-3-(5′-(5-oxo- 4,5-dihydro-1,3,4- oxadiazol-2-yl)-4-(4- (trifluoromethyl)thiazol- 2-yl)-3,3′-bipyridin-6- yl)urea | MS (ESP): 506.1 (MH⁺) for C₂₁H₁₈F₃N₇O₃S ¹H NMR (300 MHz, CD₃OD): δ 1.26 (t, 6H), 3.53 (q, 4H), 8.30 (s, 1H), 8.34 (s, 1H), 8.41 (s, 1H), 8.52 (s, 1H), 8.69 (s, 1H), 9.10 (d, 1H) ¹⁹F NMR (300 MHz, CD₃OD) −65.99 | Intermediate 73 |
| | 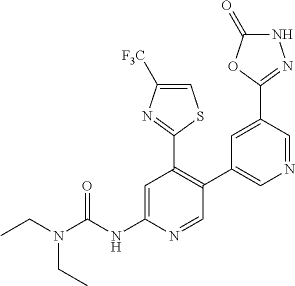 | | |
| 48 | 1-(cyclopropylmethyl)-3- (5′-(5-oxo-4,5-dihydro- 1,3,4-oxadiazol-2-yl)-4- (4- (trifluoromethyl)thiazol- 2-yl)-3,3′-bipyridin-6- yl)urea | MS (ESP): 504.0 (MH⁺) for C₂₁H₁₆F₃N₇O₃S ¹H NMR (300 MHz, CD₃OD): δ 0.28-0.32 (m, 2H), 0.52-0.56 (m, 2H), 1.09-1.20 (m, 1H), 3.20 (d, 2H), 8.00 (s, 1H), 8.36 (s, 1H), 8.41 (d, 1H), 8.48 (s, 1H), 8.80 (s, 1H), 9.16 (s, 1H) ¹⁹F NMR (300 MHz, CD₃OD) −66.01 | Intermediate 74 |
| | 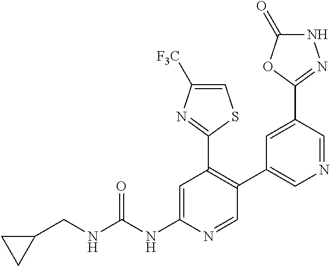 | | |
| 49 | 1-(5′-(5-oxo-4,5-dihydro- 1,3,4-oxadiazol-2-yl)-4- (4- (trifluoromethyl)thiazol- 2- yl)-3,3′-bipyridin-6-yl)- 3-(2,2,2- trifluoroethyl)urea | MS (ESP): 531.9 (MH⁺) for C₁₉H₁₁F₆N₇O₃S ¹H NMR (300 MHz, CD₃OD): δ 4.06 (q, 2H), 7.92 (s, 1H), 8.18 (t, 1H), 8.26 (d, 1H), 8.40 (d, 1H), 8.56 (d, 1H), 9.00 (d, 1H) ¹⁹F NMR (300 MHz, CD₃OD) −65.99 (s, 3F), −74.94 (t, 3F) | Intermediate 75 |
| | 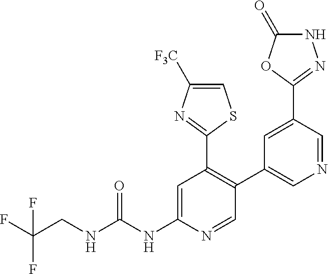 | | |
| 50 | 1-(2,2-difluoroethyl)-3- (5′-(5-oxo-4,5-dihydro- 1,3,4-oxadiazol-2-yl)-4- (4- (trifluoromethyl)thiazol- 2-yl)-3,3′-bipyridin-6- yl)urea | MS (ESP): 514.2 (MH⁺) for C₁₉H₁₂F₅N₇O₃S ¹H NMR (300 MHz, CD₃OD): δ 3.71 (td, 2H), 5.99 (tt, 1H), 7.89 (s, 1H), 8.17 (t, 1H), 8.26 (d, 1H), 8.39 (d, 1H), 8.56 (d, 1H), 9.00 (d, 1H) ¹⁹F NMR (300 MHz, CD₃OD) −65.99 (s, 3F), −125.32 (t, 1F), −125.52 (t, 1F) | Intermediate 76 |
| | 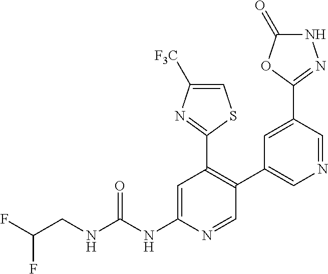 | | |

### Example 51

### 1-ethyl-3-(5′-(5-hydroxy-1,3,4-oxadiazol-2-yl)-4-(5-((2-morpholinoethylamino)methyl)-4-(trifluoromethyl)thiazol-2-yl)-3,3′-bipyridin-6-yl)urea trihydrochloride

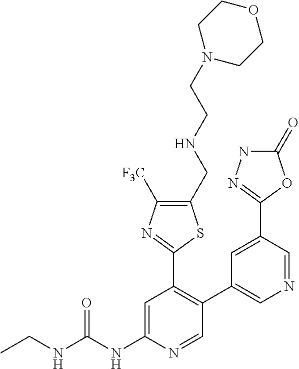

Methyl 6′-(3-ethylureido)-4′-(5-((2-morpholinoethylamino)methyl)-4-(trifluoromethyl)thiazol-2-yl)-3,3′-bipyridine-5-carboxylate (Intermediate 98, 0.35 mmol) was dissolved in tetrahydrofuran (5 mL) and saturated sodium bicarbonate solution (3 mL) was added followed by di-tert-butyl dicarbonate (0.7 mmol) and the reaction was stirred at room temperature for 96 hours at 35° C. Ethyl acetate (10 mL) was added, the layers separated and the solvent was removed in vacuo. The residue was dissolved in ethanol (20 mL), hydrazine monohydrate (1 mL) was added and the solution stirred at room temperature for 3 hours. The solvent was removed in vacuo, and the residue was twice suspended in 2:1 toluene:tetrahydrofuran (10 mL) and the solvent was removed in vacuo. This residue was then dissolved in anhydrous tetrahydrofuran (10 mL) and 1,1′-carbonyl diimidazole (500 mg) was added. The reaction was stirred at room temperature for 5 hours and the solvent was removed. The residue was chromatographed on an 8 g Analogix silica gel column eluting with 0-10% methanol in dichloromethane. The product containing fractions were combined and subjected to further HPLC purification using water and acetonitrile. The product fractions were combined, and hydrochloric acid (1 mL) was added. As the solvent was removed from the product on a rotovap at 45° C., the Boc group was cleaved to give final product (18% yield)

MS (ESP): 620.1 (M+H⁺) for C₂₆H₃₁Cl₃F₃N₉O₄S

¹H NMR (300 MHz, DMSO-d₆): δ 1.11 (t, 3H), 3.19-3.22 (m, 2H), 3.42-3.52 (m, 4H), 3.80-3.99 (m, 4H), 4.58 (s, 2H), 7.52 (bt, 1H), 8.18 (t, 1H), 8.29 (s, 1H), 8.38 (s, 1H), 8.64 (d, 1H), 8.99 (d, 1H), 9.63 (s, 1H), 12.88 (s, 1H).

### Examples 52-53

The following compounds were prepared according to the procedure described for Example 51 from the starting material as indicated in the Table.

| Ex | Compound | Data | SM |
|---|---|---|---|
| 52 | 1-(4-(5- ((cyclohexylamino)methyl)- 4- (trifluoromethyl)thiazol- 2-yl)-5′-(5-hydroxy-1,3,4- oxadiazol-2-yl)-3,3′- bipyridin-6-yl)-3- ethylurea hydrochloride | MS (ESP): 464.1 (MH⁺) for C₁₈H₁₂F₃N₇O₃S ¹H NMR (300 MHz, CD₃OD): δ 2.90 (s, 3H), 7.81 (s, 1H), 8.16 (t, 1H), 8.25 (d, 1H), 8.37 (d, 1H), 8.54 (d, 1H), 9.00 (d, 1H) ¹⁹F NMR (300 MHz, CD₃OD) −65.99 | Intermediate 101 |
| | 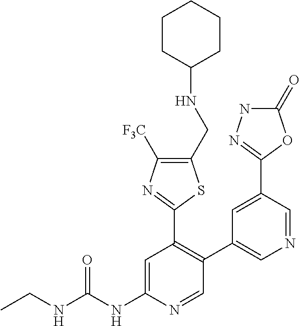 | | |
| 53 | 1-(4-(5- ((cyclopentylamino)methyl)- 4- (trifluoromethyl)thiazol- 2-yl)-5′-(5-hydroxy-1,3,4- oxadiazol-2-yl)-3,3′- bipyridin-6-yl)-3- ethylurea hydrochloride | MS (ESP): 575.1 (M + H⁺) for C₂₅H₂₆ClF₃N₈O₃S; ¹H NMR (300 MHz, DMSO-d₆): δ 1.11 (t, 3H), 1.51 (m, 2H), 1.52- 1.76 (m, 4H), 1.93 (m, 2H), 1.72- 1.76 (m, 2H), 3.18-3.22 (m, 2H), 3.49 (m, 1H), 4.49 (m, 2H), 7.52 (m, 1H), 8.18 (t, 1H), 8.27 (s, 1H), 8.38 (s, 1H), 8.65 (d, 1H), 9.00 (d, 1H), 9.61 (m, 2H), 12.88 (bs, 1H). | Intermediate 102 |
| | 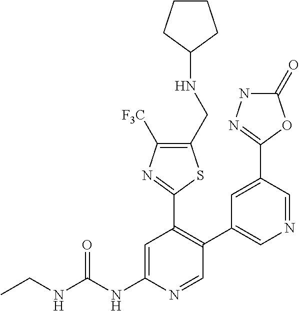 | | |

### Example 54

### 1-ethyl-3-(4-(5-((2-methoxyethylamino)methyl)-4-(trifluoromethyl)thiazol-2-yl)-5′-(5-methyl-1,3,4-oxadiazol-2-yl)-3,3′-bipyridin-6-yl)urea

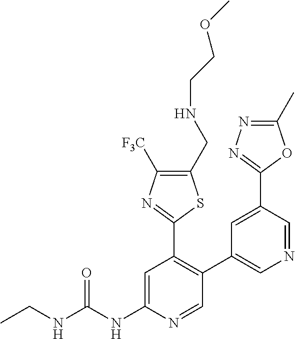

Methyl 6′-(3-ethylureido)-4′-(5-((2-methoxyethylamino)methyl)-4-(trifluoromethyl)thiazol-2-yl)-3,3′-bipyridine-5-carboxylate (Intermediate 97, 200 mg) was dissolved in tetrahydrofuran (3 mL) and methanol (3 mL). 1N Sodium hydroxide (3 mL) was added, and the reaction mixture was stirred at room temperature for 3 h. The organics were removed and the residual aqueous phase was acidified to pH ˜2 with 1N hydrochloric acid. The water was then removed in vacuo. The residue was dissolved in phosphorous oxychloride (3 mL), acetic hydrazide (200 mg) was added and the solution heated at 60° C. for 3 hours. Most of the phosphorous oxychloride was removed in vacuo and then saturated sodium bicarbonate was added to pH ˜7. The solution was extracted with 2:1 ethyl acetate: tetrahydrofuran (3×, 3 mL each). The organic phases were combined, dried over sodium sulfate, and the solvent was removed in vacuo. The crude product was chromatographed on a 4 g Analogix silica gel column using 0-10% methanol in dichloromethane.

MS (ESP): 563.1 (M+H⁺) for C₂₄H₂₅F₃N₈O₃S

¹H NMR (300 MHz, DMSO-d₆): δ 1.22 (t, 3H), 2.63 (s, 3H), 2.71 (t, 2H), 3.31 (s, 3H), 3.31-3.41 (m, 4H), 4.07 (d, 2H), 7.79 (s, 1H), 8.34-8.36 (m, 2H), 8.63 (d, 1H), 9.17 (d, 1H).

### Examples 55-58

The following Examples were prepared according to the procedure described for Example 54 from the starting materials indicated in the Table.

| Ex | Compound | Data | SM |
|---|---|---|---|
| 55 | 1-ethyl-3-(5′-(5-methyl- 1,3,4-oxadiazol-2-yl)-4- (5-((2- morpholinoethylamino) methyl)-4- (trifluoromethyl)thiazol- 2-yl)-3,3′-bipyridin-6- yl)urea | MS (ESP): 618.3 (M + H⁺) for C₂₇H₃₀F₃N₉O₃S; ¹H NMR (300 MHz, CDCl₃): δ 1.27 (t, 3H), 2.66 (s, 3H), 2.66-2.72 (bs, 4H), 2.82-2.88 (m, 2H), 3.42-3.48 (m, 2H), 3.77-3.84 (m, 4H), 4.10 (d, 2H), 5.05 (bs, 1H), 7.62 (s, 1H), 8.23 (s, 1H), 8.30 (t, 1H), 8.59 (d, 1H), 8.98 (bs, 1H), 9.24 (d, 1H), 9.64 (s, 1H) | Intermediate 98 |
| | 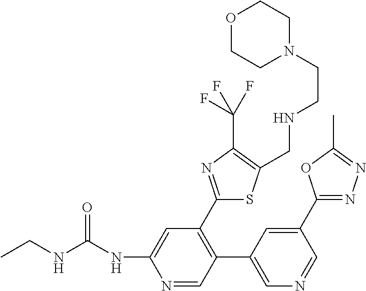 | | |
| 56 | 1-(4-(5- ((cyclohexylamino)methyl)- 4- (trifluoromethyl)thiazol- 2-yl)-5′-(5-methyl-1,3,4- oxadiazol-2-yl)-3,3′- bipyridin-6-yl)-3- ethylurea | MS (ESP): 587.1 (M + H⁺) for C₂₇H₂₉F₃N₈O₂S; ¹H NMR (300 MHz, CD₃OD): δ 0.97-1.18 (m, 2H), 1.19-1.25 (m, 3H), 1.22 (t, 3H), 1.59-1.79 (m, 5H), 2.32-2.37 (m, 1H), 2.64 (s, 3H), 3.30-3.38 (m, 2H), 4.05 (m, 2H), 7.82 (s, 1H), 8.35-8.38 (m, 2H), 8.65 (d, 1H), 9.19 (d, 1H). | Intermediate 101 |
| | 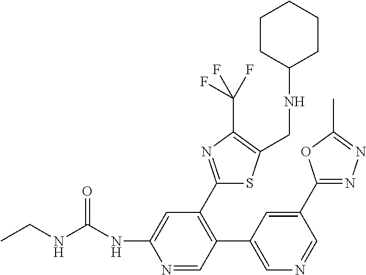 | | |
| 57 | 1-(4-(5- ((cyclopentylamino)methyl)- 4- (trifluoromethyl)thiazol- 2-yl)-5′-(5-methyl-1,3,4- oxadiazol-2-yl)-3,3′- bipyridin-6-yl)-3- ethylurea | MS (ESP): 573.3 (M + H⁺) for C₂₆H₂₇F₃N₈O₂S; ¹H NMR (300 MHz, CD₃OD): δ 1.22 (t, 3H), 1.23-1.31 (m, 2H), 1.48-1.52 (m, 2H), 1.61-1.65 (m, 2H), 1.72-1.76 (m, 2H), 2.64 (s, 3H), 3.02-3.06 (m, 1H), 3.31-3.39 (m, 2H), 4.03 (m, 2H), 7.81 (m, 1H), 8.34-8.37 (m, 2H), 8.64 (d, 1H), 9.17 (d, 1H). | Intermediate 102 |
| | 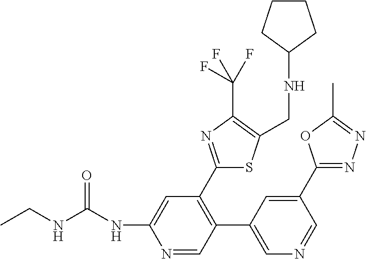 | | |
| 58 | 1-ethyl-3-(5′-(5-methyl- 1,3,4-oxadiazol-2-yl)-4- (5-((tetrahydro-2H-pyran- 4-ylamino)methyl)-4- (trifluoromethyl)thiazol- 2-yl)-3,3′-bipyridin-6- yl)urea | MS (ESP): 589.2 (M + H⁺) for C₂₆H₂₇F₃N₈O₃S; ¹H NMR (300 MHz, DMSO-d₆): δ 1.29 (t, 3H), 1.24-1.38 (m, 2H), 1.69-1.78 (m, 2H), 2.60-2.69 (m, 1H), 2.67 (s, 3H), 3.30-3.38 (m, 2H), 3.43-3.52 (m, 2H), 3.90-3.96 (m, 2H), 4.07 (d, 2H), 7.45 (s, 1H), 8.25 (s, 1H), 8.35 (t, 1H), 8.61 (d, 1H), 8.85 (bs, 1H), 9.01 (bs, 1H), 9.26 (d, 1H). | Intermediate 103 |
| | 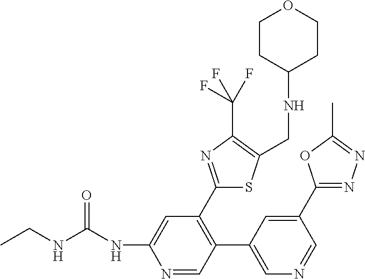 | | |

### Example 59

### 2-(6-(3-ethylureido)-5′-(5-oxo-4,5-dihydro-1,3,4-oxadiazol-2-yl)-3,3′-bipyridin-4-yl)-N-(2-methoxyethyl)-4-(trifluoromethyl)thiazole-5-carboxamide

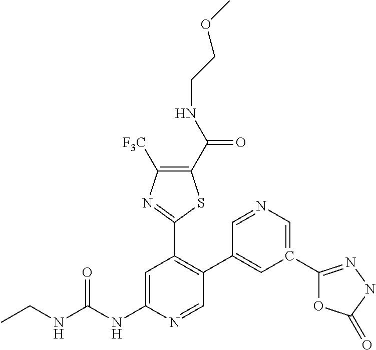

To a 50 mL round bottom flask was added methyl 6′-(3-ethylureido)-4′-(5-(2-methoxyethylcarbamoyl)-4-(trifluoromethyl)thiazol-2-yl)-3,3′-bipyridine-5-carboxylate (Intermediate 104, 80 mg), ethanol (10 mL) and hydrazine (0.3 mL) and the solution heated to reflux for 3 h. The solvent was removed in vacuo and the residue evaporated from toluene (3×, 60 mL) to remove excess hydrazine. The yellowish gum was then dried at 50° C. in a vacuum oven overnight. The crude solid was re-dissolved in tetrahydrofuran (10 mL), then triethylamine (1 mL) and 1,1′-carbonyl diimidazole (0.5 g) were added. The solution was then stirred at room temperature for 2 hrs. The solvent was removed under reduced pressure, DIUF water (10 mL) was added, and the mixture was stirred for 30 min. A white solid precipitated out which was filtered and dried to give 40 mg product as off-white solid.

MS (ESP): 579.0 (M+H⁺) for C₂₃H₂₁F₃N₈O₅S

¹H NMR (300 MHz, DMSO-d₆): δ 1.11 (t, 3H), 3.21 (m, 2H), 3.24 (s, 3H), 3.36 (m, 4H), 7.01 and 7.64 (bs, tautomers, 1H), 7.49 (t, 1H), 8.20 (m, 1H), 8.26 (s, 1H), 8.37 (s, 1H), 8.64 (d, 1H), 9.01 (m, 2H), 9.52 (bs, 1H).

### Example 60

### 2-(6-(3-ethylureido)-5′-(5-methyl-1,3,4-oxadiazol-2-yl)-3,3′-bipyridin-4-yl)-N-(2-methoxyethyl)-4-(trifluoromethyl)thiazole-5-carboxamide

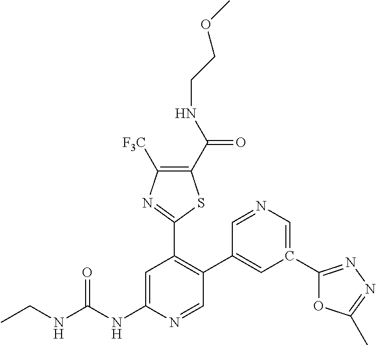

To a 50 mL round bottom flask was added crude 6′-(3-ethylureido)-4′-(5-(2-methoxyethylcarbamoyl)-4-(trifluoromethyl)thiazol-2-yl)-3,3′-bipyridine-5-carboxylic acid (Intermediate 105, 90 mg), phosphorus oxychloride (3 mL) and acetyl hydrazine (0.2 g). The solution was then heated to 65° C. for 3 h after which time no starting material remained by LC/MS (LC purity was ˜40%). The solvent was removed under reduced pressure and toluene (3×, 60 mL) was added to remove excess phosphorus oxychloride. Saturated sodium bicarbonate was then added until pH ˜7. The solution was extracted with ethyl acetate (3×, 100 mL). The combined organics were dried over sodium sulfate, filtered, and concentrated. The residue was dissolved in methanol and purified by prep HPLC to give 20 mg light yellow solid.

MS (ESP): 577.2 (M+H⁺) for C₂₄H₂₃F₃N₈O₄S

¹H NMR (300 MHz, CD₃OD): δ 1.22 (t, 3H), 2.35 and 2.64 (s, 3H), 3.30 (m, 6H), 3.47 (s, 3H), 7.91 (s, 1H), 8.39 (m, 2H), 8.67 (d, 1H), 9.21 (s, 1H).

### Example 61

### 2-(6-(3-ethylureido)-5′-(5-oxo-4,5-dihydro-1,3,4-oxadiazol-2-yl)-3,3′-bipyridin-4-yl)-N-(2-morpholinoethyl)-4-(trifluoromethyl)thiazole-5-carboxamide

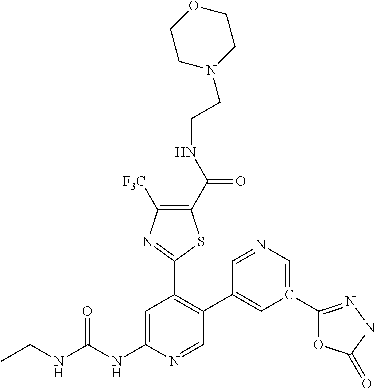

To a 50 mL round bottom flask was added methyl 6′-(3-ethylureido)-4′-(5-(2-morpholinoethylcarbamoyl)-4-(trifluoromethyl)thiazol-2-yl)-3,3′-bipyridine-5-carboxylate (Intermediate 106, 200 mg), ethanol (10 mL) and hydrazine (0.5 mL) and the solution heated to reflux for 3 h. The solvent was removed in vacuo and the residue evaporated from toluene (3×, 60 mL) to remove excess hydrazine. The crude intermediate was then dried at 50° C. in a vacuum oven overnight. The solid was re-dissolved in tetrahydrofuran (10 mL), then triethylamine (1 mL) and 1,1′-carbonyl diimidazole (0.5 g) were added. The solution was then allowed to stir at room temperature for 2 hrs. The solvent was removed under reduced pressure and water (10 mL) was added. The mixture was stirred at room temperature overnight however no product precipitated. The crude solution was then purified on a 30 g Analogix C18 column (water/methanol: 40% MeOH/H₂O) to give 60 mg light yellow solid.

MS (ESP): 634.2 (M+H⁺) for C₂₆H₂₆F₃N₉O₅S

¹H NMR (300 MHz, DMSO-d₆): δ 1.11 (t, 3H), 2.36 (m, 8H), 3.21 (m, 2H), 3.52 (m, 4H), 7.02 and 7.63 (bs, tautomers, 1H), 7.51 (t, 1H), 8.17 (t, 1H), 8.27 (s, 1H), 8.36 (s, 1H), 8.62 (d, 1H), 8.85 (t, 1H), 8.99 (d, 1H), 9.53 (s, 1H).

### Example 62

### 2-(6-(3-ethylureido)-5′-(5-methyl-1,3,4-oxadiazol-2-yl)-3,3′-bipyridin-4-yl)-N-(2-morpholinoethyl)-4-(trifluoromethyl)thiazole-5-carboxamide

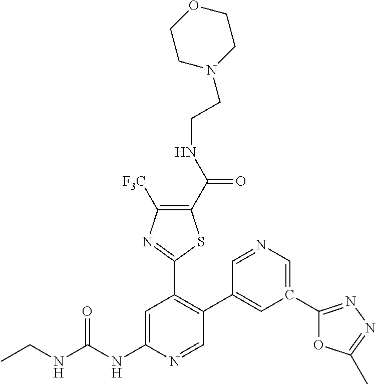

To a 50 mL round bottom flask was added crude 6′-(3-ethylureido)-4′-(5-(2-morpholinoethylcarbamoyl)-4-(trifluoromethyl)thiazol-2-yl)-3,3′-bipyridine-5-carboxylic acid (Intermediate 107, 200 mg), phosphorus oxychloride (3 mL) and acetyl hydrazine (0.2 g) and the solution heated to 65° C. for 3 h. The solvent was removed under reduced pressure and toluene (3×, 60 mL) was added to remove excess phosphorus oxychloride. Saturated sodium bicarbonate was added until pH ˜7 and the solution was extracted with ethyl acetate (3×, 100 mL). The combined organics were dried over sodium sulfate, filtered, and concentrated. The residue was then dissolved in methanol and purified by prep HPLC to give 60 mg light yellow solid.

MS (ESP): 632.1 (M+H⁺) for C₂₇H₂₈F₃N₉O₄S

¹H NMR (300 MHz, CD₃OD): δ 1.22 (t, 3H), 2.50 (m, 6H), 2.62 and 2.64 (s, 3H), 3.34 (m, 2H), 3.44 (t, 2H), 3.64 (t, 4H), 7.92 (s, 1H), 8.41 (m, 2H), 8.68 (d, 1H), 9.21 (s, 1H).

### Example 63

### 2-(6-(3-ethylureido)-5′-(5-oxo-4,5-dihydro-1,3,4-oxadiazol-2-yl)-3,3′-bipyridin-4-yl)-N-(2-(4-methylpiperazin-1-yl)ethyl)-4-trifluoromethyl)thiazole-5-carboxamide

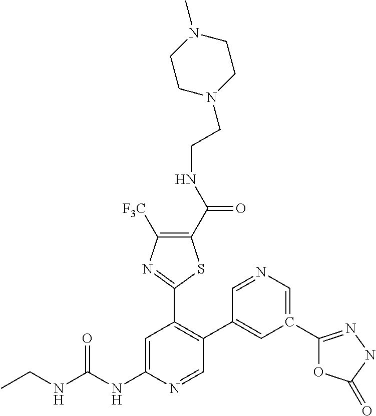

To a 50 mL round bottom flask was added methyl 6′-(3-ethylureido)-4′-(5-(2-(4-methylpiperazin-1-yl)ethylcarbamoyl)-4-(trifluoromethyl)thiazol-2-yl)-3,3′-bipyridine-5-carboxylate (Intermediate 108, 300 mg), ethanol (10 mL) and hydrazine (0.5 mL) and the solution heated to reflux for 3 hrs. The solvent was removed in vacuo and the residue evaporated from toluene (3×, 60 mL) to remove excess hydrazine. The crude yellowish gum was then dried at 50° C. in a vacuum oven overnight. The crude solid was re-dissolved in tetrahydrofuran (10 mL), 1,1′-carbonyl diimidazole (0.5 g) was added and the solution stirred at room temperature for 2 hrs. The solvent was then removed in vacuo. Water (10 mL) was added and the mixture was then left to stir overnight however no product precipitated. The product was purified by prep HPLC (water/acetonitrile) to give 150 mg off-white solid.

MS (ESP): 647.1 (M+H⁺) for C₂₇H₂₉F₃N₁₀O₄S

¹H NMR (300 MHz, DMSO-d₆): δ 1.11 (t, 3H), 2.15 (s, 3H), 2.38 (m, 10H), 3.21 (m, 4H), 7.50 (t, 1H), 8.20 (t, 1H), 8.27 (s, 1H), 8.36 (s, 1H), 8.64 (d, 1H), 8.81 (t, 1H), 9.00 (d, 1H), 9.52 (bs, 1H).

### Example 64

### 2-(6-(3-ethylureido)-5′-(5-methyl-1,3,4-oxadiazol-2-yl)-3,3′-bipyridin-4-yl)-N-(2-(4-methylpiperazin-1-yl)ethyl)-4-(trifluoromethyl)thiazole-5-carboxamide

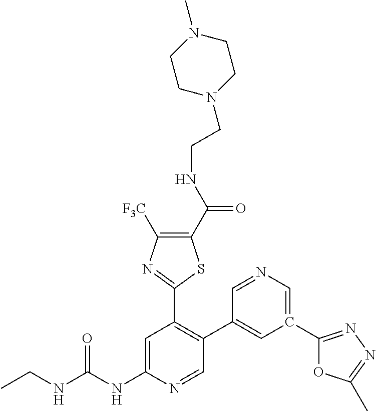

To a 50 mL round bottom flask was added crude 6′-(3-ethylureido)-4′-(5-(2-(4-methylpiperazin-1-yl)ethylcarbamoyl)-4-(trifluoromethyl)thiazol-2-yl)-3,3′-bipyridine-5-carboxylic acid (Intermediate 109, 200 mg), phosphorus oxychloride (3 mL) and acetyl hydrazine (0.2 g). The solution was then heated at 65° C. for 3 h. The solvent was removed in vacuo and the residue evaporated from toluene (3×, 60 mL) to remove excess phosphorus oxychloride. Saturated sodium bicarbonate was added to pH ˜7 and the solution was extracted with ethyl acetate/tetrahydrofuran (1/1) (5×, 100 mL). The combined organics were dried over sodium sulfate, filtered, and concentrated. The residue was then dissolved in methanol and purified by prep HPLC to give 30 mg pale yellow solid.

MS (ESP): 645.3 (M+H⁺) for C₂₈H₃₁F₃N₁₀O₃S

¹H NMR (300 MHz, DMSO-d₆): δ 1.11 (t, 3H), 2.44 (s, 3H), 2.58 (m, 10H), 2.60 (s, 3H), 3.21 (m, 4H), 7.47 (t, 1H), 8.28 (s, 1H), 8.36 (s, 1H), 8.41 (s, 1H), 8.71 (s, 1H), 8.87 (t, 1H), 9.18 (s, 1H), 9.54 (bs, 1H).

### Example 65

### N-cyclopropyl-2-(6-(3-ethylureido)-5′-(5-oxo-4,5-dihydro-1,3,4-oxadiazol-2-yl)-3,3′-bipyridin-4-yl)-4-(trifluoromethyl)triazole-5-carboxamide

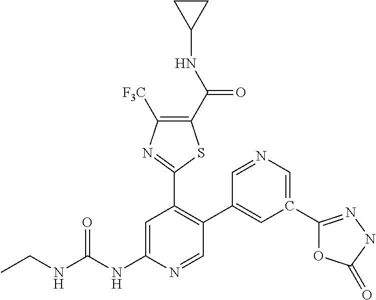

To a 50 mL round bottom flask was added methyl 4′-(5-(cyclopropylcarbamoyl)-4-(trifluoromethyl)thiazol-2-yl)-6′-(3-ethylureido)-3,3′-bipyridine-5-carboxylate (Intermediate 110, 300 mg), ethanol (10 mL) and hydrazine (0.5 mL) and the solution was heated to reflux for 3 hrs. The solvent was removed in vacuo and the residue evaporated from toluene (3×, 60 mL) to remove excess hydrazine. The crude dark yellowish gum was then dried at 50° C. in a vacuum oven overnight. The crude solid was re-dissolved in tetrahydrofuran (10 mL), 1,1′-carbonyl diimidazole (0.5 g) was added and the solution was stirred at room temperature for 2 hrs. The solvent was removed under reduced pressure, water (10 mL) was added and the mixture left to stir at room temperature overnight. The yellow solids were filtered and washed with water to give 80 mg product at ˜80% purity. The material was further purified by prep HPLC (water/acetonitrile) to give 30 mg white solid.

MS (ESP): 561.3 (M+H⁺) for C₂₃H₁₉F₃N₈O₄S

¹H NMR (300 MHz, DMSO-d₆): δ 0.48 (m, 2H), 0.69 (m, 2H), 1.11 (t, 3H), 2.76 (m, 1H), 3.22 (m, 2H), 7.78 (t, 1H), 8.19 (s, 1H), 8.24 (s, 1H), 8.37 (s, 1H), 8.63 (s, 1H), 9.00 (s, 2H), 9.52 (bs, 1H).

### Example 66

### N-cyclopropyl-2-(6-(3-ethylureido)-5′-(5-methyl-1,3,4-oxadiazol-2-yl)-3,3′-bipyridin-4-yl)-4-(trifluoromethyl)thiazole-5-carboxamide

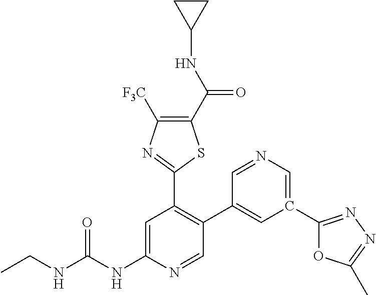

To a 50 mL round bottom flask was added crude 4′-(5-(cyclopropylcarbamoyl)-4-(trifluoromethyl)thiazol-2-yl)-6′-(3-ethylureido)-3,3′-bipyridine-5-carboxylic acid (Intermediate 111, 200 mg), phosphorus oxychloride (3 mL) and acetyl hydrazine (0.2 g). The solution was then heated at 65° C. for 3 h. The solvent was removed in vacuo and the residue evaporated from toluene (3×, 60 mL) to remove excess phosphorus oxychloride. Saturated sodium bicarbonate was added to pH ˜7 and the solution was extracted with ethyl acetate (3×, 100 mL). The combined organics were dried over sodium sulfate, filtered, and concentrated. The residue was then dissolved in methanol and purified by prep HPLC to give 40 mg pale yellow solid.

MS (ESP): 559.2 (M+H⁺) for C₂₄H₂₁F₃N₈O₃S

¹H NMR (300 MHz, DMSO-d₆): δ 0.47 (m, 2H), 0.69 (m, 2H), 1.11 (t, 3H), 2.60 (s, 3H), 2.74 (m, 1H), 3.22 (m, 2H), 7.47 (t, 1H), 8.24 (s, 1H), 8.34 (t, 1H), 8.41 (s, 1H), 8.69 (d, 1H), 8.99 (d, 1H), 9.17 (d, 1H), 9.53 (bs, 1H).

### Example 67

### N-cyclopentyl-2-(6-(3-ethylureido)-5′-(5-oxo-4,5-dihydro-1,3,4-oxadiazol-2-yl)-3,3′-bipyridin-4-yl)-4-(trifluoromethyl)thiazole-5-carboxamide

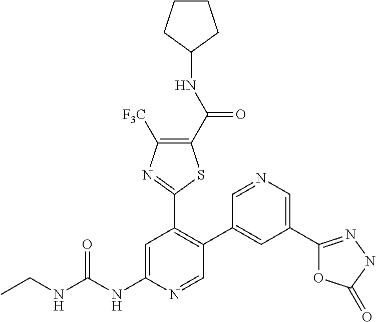

To a 50 mL round bottom flask was added methyl 4′-(5-(cyclopentylcarbamoyl)-4-(trifluoromethyl)thiazol-2-yl)-6′-(3-ethylureido)-3,3′-bipyridine-5-carboxylate (Intermediate 112, 200 mg), ethanol (10 mL) and hydrazine (1.0 mL). The solution was heated to reflux for 3 hrs. The solvent was removed in vacuo and the residue evaporated from toluene (3×, 60 mL) to remove excess hydrazine. The crude dark yellowish gum was then dried at 50° C. in a vacuum oven overnight. The crude solid was re-dissolved in tetrahydrofuran (10 mL), and 1,1′-carbonyl diimidazole (0.5 g) was added. The solution was then allowed to stir at room temperature for 18 hrs. The solvent was removed under reduced pressure, water (10 mL) was added and the mixture was then stirred at room temperature for 3 hours. A yellow solid precipitated out and was filtered and triturated with acetonitrile to give 84 mg of pale yellow solid.

MS (ESP): 589.2 (M+H⁺) for C₂₅H₂₃F₃N₈O₄S

¹H NMR (300 MHz, DMSO-d₆): δ 1.11 (t, 3H), 1.35-1.64 (m, 6H), 1.80-1.99 (m, 2H), 3.16-3.23 (m, 2H), 4.06-4.12 (m, 1H), 7.48 (bt, 1H), 8.20 (t, 1H), 8.24 (s, 1H), 8.38 (s, 1H), 8.63 (d, 1H), 8.91 (d, 1H), 9.00 (d, 1H), 9.53 (bs, 1H)

### Example 68

### N-cyclopentyl-2-(6-(3-ethylureido)-5′-(5-methyl-1,3,4-oxadiazol-2-yl)-3,3′-bipyridin-4-yl)-4-(trifluoromethyl)thiazole-5-carboxamide

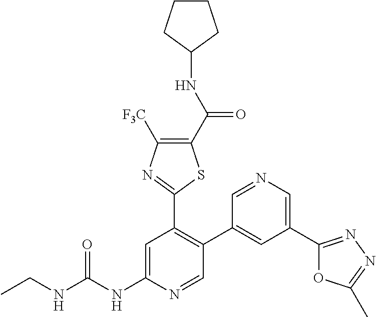

To a 20 mL vial was added crude 4′-(5-(cyclopentylcarbamoyl)-4-(trifluoromethyl)thiazol-2-yl)-6′-(3-ethylureido)-3,3′-bipyridine-5-carboxylic acid (Intermediate 113, 150 mg), phosphorus oxychloride (3 mL) and acetyl hydrazine (0.2 g). The solution was then heated at 60° C. for 3 h. The solvent was removed in vacuo and the residue evaporated from toluene (3×, 60 mL) to remove excess phosphorus oxychloride. Saturated sodium bicarbonate was added to pH ˜7 and the solution extracted with ethyl acetate (3×, 10 mL). The combined organics were dried over sodium sulfate, filtered, and concentrated. The residue was then chromatographed on a 4 g Analogix silica gel column using 0-10% methanol in dichloromethane to give 42 mg of pale yellow solid.

MS (ESP): 587.1 (M+H⁺) for C₂₆H₂₅F₃N₈O₃S

¹H NMR (300 MHz, DMSO-d₆): δ 1.11 (t, 3H), 1.39-1.61 (m, 6H), 1.79-1.83 (m, 2H), 2.61 (s, 3H), 3.18-3.25 (m, 2H), 4.02-4.16 (m, 1H), 7.48 (bt, 1H), 8.24 (s, 1H), 8.35 (t, 1H), 8.41 (d, 1H), 8.69 (d, 1H), 8.90 (d, 1H), 9.17 (d, 1H), 9.55 (bs, 1H).

### Example 69

### N-cyclohexyl-2-(6-(3-ethylureido)-5′-(5-oxo-4,5-dihydro-1,3,4-oxadiazol-2-yl)-3,3′-bipyridin-4-yl)-4-(trifluoromethyl)thiazole-5-carboxamide

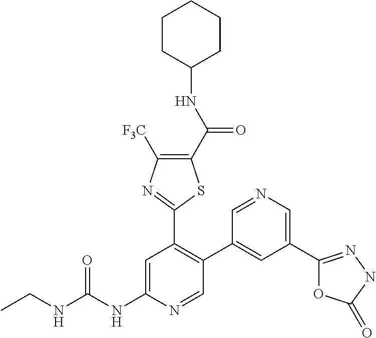

To a 50 mL round bottom flask was added methyl 4′-(5-(cyclohexylcarbamoyl)-4-(trifluoromethyl)thiazol-2-yl)-6′-(3-ethylureido)-3,3′-bipyridine-5-carboxylate (Intermediate 114, 200 mg), ethanol (10 mL) and hydrazine (0.5 mL). The solution was then heated to reflux for 3 hrs. The solvent was removed in vacuo and the residue evaporated from toluene (3×, 60 mL) to remove excess hydrazine. The crude yellowish gum was then dried at 50° C. in a vacuum oven overnight. The crude solid was re-dissolved in tetrahydrofuran (10 mL), 1,1′-carbonyl diimidazole (0.5 g) was added and the solution allowed to stir at room temperature for 2 hrs. The solvent was removed under reduced pressure, water (10 mL) was added and the mixture stirred at room temperature for 2 h. The solids were removed by filtration, triturated with acetonitrile and dried in a vacuum oven at 50° C. for 18 hours to give 73 mg pale yellow solid.

MS (ESP): 603.3 (M+H⁺) for C₂₆H₂₅F₃N₈O₄S

¹H NMR (300 MHz, DMSO-d₆): δ 1.11 (t, 3H), 1.12-1.36 (m, 4H), 1.53-1.60 (m, 1H), 1.60-1.78 (m, 5H), 3.16-3.25 (m, 2H), 3.60-3.65 (m, 1H), 7.48 (bt, 1H), 8.21 (t, 1H), 8.24 (s, 1H), 8.37 (s, 1H), 8.63 (d, 1H), 8.84 (d, 1H), 9.00 (d, 1H), 9.53 (s, 1H).

### Example 70

### N-cyclohexyl-2-(6-(3-ethylureido)-5′-(5-methyl-1,3,4-oxadiazol-2-yl)-3,3′-bipyridin-4-yl)-4-(trifluoromethyl)thiazole-5-carboxamide

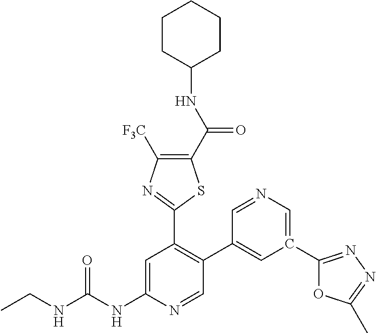

To a 50 mL round bottom flask was added crude 4′-(5-(cyclohexylcarbamoyl)-4-(trifluoromethyl)thiazol-2-yl)-6′-(3-ethylureido)-3,3′-bipyridine-5-carboxylic acid (Intermediate 115, 200 mg), phosphorus oxychloride (3 mL) and acetyl hydrazine (0.2 g). The solution was then heated at 60° C. for 3 h. The solvent was removed in vacuo and the residue evaporated from toluene (3×, 60 mL) to remove excess phosphorus oxychloride. Saturated sodium bicarbonate was added to pH ˜7 and the solution extracted with ethyl acetate (3×, 20 mL). The combined organics were dried over sodium sulfate, filtered, and concentrated. The residue was then chromatographed on a 4 g Analogix silica gel column using 0-10% methanol in dichloromethane to give 53 mg pale yellow solid.

MS (ESP): 601.2 (M+H⁺) for C₂₇H₂₇F₃N₈O₃S

¹H NMR (300 MHz, DMSO-d₆): δ 1.11 (t, 3H), 1.12-1.29 (m, 6H), 1.50-1.60 (m, 1H), 1.60-1.78 (m, 3H), 2.60 (s, 3H), 3.16-3.26 (m, 2H), 3.60-3.65 (m, 1H), 7.49 (bt, 1H), 8.25 (s, 1H), 8.35 (t, 1H), 8.41 (s, 1H), 8.69 (d, 1H), 8.83 (d, 1H), 9.17 (d, 1H), 9.55 (bs, 1H).

### Examples 71-72

The following Examples were prepared according to the general procedures described below from the starting materials indicated in the Table.

### General Procedure

A methyl ester (0.2 mmol) was suspended in ethanol (10 mL) and anhydrous hydrazine (0.1 mL) was added. The resulting suspension was heated at reflux for 3 h. The solvent was removed in vacuo. Toluene (5 mL) was added to the residue and removed in vacuo twice to remove traces of hydrazine. Anhydrous tetrahydrofuran (10 mL) and 1,1′-carbonyl diimidazole (100 mg) were added, and the reaction was stirred at room temperature for 16 h. The solvent was removed in vacuo and the residue subjected to reverse phase chromatography using 10-99% acetonitrile in water to isolate the product.

| Ex | Compound | Data | SM |
|---|---|---|---|
| 71 | 1-ethyl-3-(2′-ethyl-5′-(5- hydroxy-1,3,4-oxadiazol- 2-yl)-4-(4- (trifluoromethyl)thiazol- 2-yl)-3,3′-bipyridin-6- yl)urea | MS (ESP): 506.1 (MH⁺) for C₂₁H₁₈F₃N₇O₃S ¹H NMR (300 MHz, DMSO-d₆): 1.05 (t, 3H), 1.22 (t, 1H), 2.51-2.57 (m, 2H), 3.280-3.39 (m, 2H), 8.02 (s, 1H), 8.05 (d, 1H), 8.18 (d, 1H), 8.26 (d, 1H), 9.00 (d, 1H) | Intermediate 120 |
| | 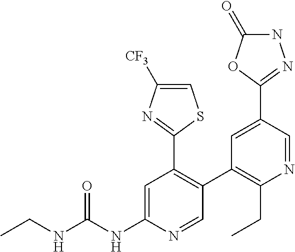 | | |
| 72 | 1-ethyl-3-(2′-ethyl-5′-(5- hydroxy-1,3,4-oxadiazol- 2-yl)-4-(4-phenylthiazol- 2-yl)-3,3′-bipyridin-6- yl)urea | MS (ESP): 514.2(MH⁺) for C₂₆H₂₃N₇O₃S ¹H NMR (300 MHz, DMSO-d₆): 1.06 (t, 3H), 1.24 (t, 1H), 2.58-2.62 (m, 2H), 3.30-3.40 (m, 2H), 7.31- 7.34 (m, 3H), 7.60-7.69 (m, 2H), 7.86 (s, 1H), 8.01 (s, 1H), 8.08 (d, 1H), 8.24 (d, 1H), 9.00 (d, 1H) | Intermediate 121 |
| | 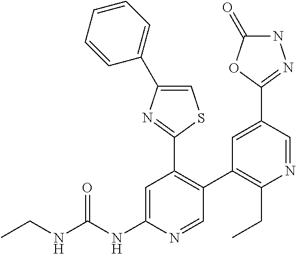 | | |

### Examples 73-74

The following Examples were prepared according to the general procedures described below from the starting materials indicated in the Table.

### General Procedure

The appropriate carboxylic acid (0.1 mmol) and acetic hydrazide (0.15 mmol) were suspended in phosphorous oxychloride (3 mL). The reaction was heated at 65° C. for 3 h. The phosphorous oxychloride was removed in vacuo and toluene (5 mL) was added and also removed in vacuo. Saturated sodium bicarbonate solution (10 mL) was added and the suspension was extracted with 2:1 ethyl acetate:tetrahydrofuran (3×, 5 mL). The organic phases were combined and the solvent was removed in vacuo. The residue was dissolved twice in methyl tert-butyl ether (5 mL each) and the solvent removed in vacuo to remove trace solvents. The residue was given a final trituration with methyl tert-butyl ether (5 mL) and filtered to give the appropriate methyl oxadiazole.

| Ex | Compound | Data | SM |
|---|---|---|---|
| 73 | 1-ethyl-3-(2′-ethyl-5′-(5- methyl-1,3,4-oxadiazol- 2-yl)-4-(4- (trifluoromethyl)thiazol- 2-yl)-3,3′-bipyridin-6- yl)urea | MS (ESP): 504.0 (MH⁺) C₂₂H₂₀F₃N₇O₂S ¹H NMR (300 MHz, DMSO-d₆): 1.01 (t, 3H), 1.12 (t, 1H), 2.42-2.53 (m, 2H), 2.58 (s, 3H), 3.20-3.26 (m, 2H), 7.61 (bt, 1H), 8.19 (d, 1H), 8.30 (s, 1H), 8.37 (s, 1H), 9.20 (d, 1H), 9.48 (bs, 1H) | Intermediate 122 |
| | 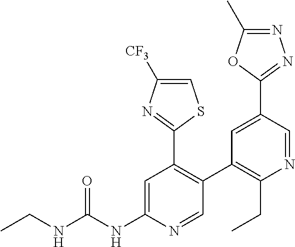 | | |
| 74 | 1-ethyl-3-(2′-ethyl-5′-(5- hydroxy-1,3,4-oxadiazol- 2-yl)-4-(4-phenylthiazol- 2-yl)-3,3′-bipyridin-6- yl)urea | MS (ESP): 512.1 (MH⁺) for C₂₇H₂₅N₇O₂S ¹H NMR (300 MHz, DMSO-d₆): 1.02 (t, 3H), 1.12 (t, 1H), 2.41-2.56 (m, 2H), 2.58 (s, 3H), 3.20-3.31 (m, 2H), 7.34-7.38 (m, 3H), 7.68-7.72 (m, 3H), 8.17 (d, 1H), 8.19 (s, 1H), 8.27 (s, 1H), 8.39 (s, 1H), 9.19 (d, 1H), 9.48 (bs, 1H) | Intermediate 123 |
| | 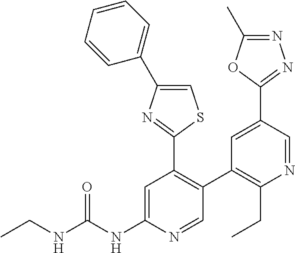 | | |

### Example 75

### 1-(2′-ethoxy-6′-(5-oxo-4,5-dihydro-1,3,4-oxadiazol-2-yl)-4-(4-(trifluoromethyl)thiazol-2-yl)-3,4′-bipyridin-6-yl)-3-ethylurea

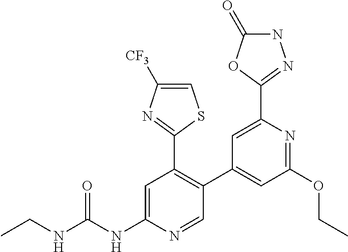

To a 100 mL round bottom flask was charged methyl 6′-ethoxy-6-(3-ethylureido)-4-(4-(trifluoromethyl)thiazol-2-yl)-3,4′-bipyridine-2′-carboxylate (Intermediate 145, 160 mg, 0.323 mmol) with ethanol (20 mL). Hydrazine monohydrate (4 mL) was added and the mixture was heated to reflux for 1 h. After concentrating under reduced pressure, the crude product was further dried in vacuum oven at 50° C. for overnight.

To the crude product was charged tetrahydrofuran (30 mL) with 1,1′-carbonyl diimidazole (160 mg, 0.97 mmol) and the mixture was stirred at room temperature for 0.5 h. Starting material remained so another portion of 1,1′-carbonyl diimidazole (110 mg) was added and the mixture stirred for another 1 h. After concentration under reduced pressure, the crude product was triturated with water. The precipitate was collected by filtration and dried in oven at 60° C. to give a beige solid (140 mg, 83.3% over two steps).

MS (ESP): 522.0 (MH⁺) for C₂₁H₁₈F₃N₇O₄S

¹H NMR (300 MHz, CD₃OD): δ 1.22 (t, 3H), 1.39 (t, 3H), 3.31 (q, 2H), 4.45 (q, 2H), 6.86 (d, 1H), 7.32 (d, 1H), 7.79 (s, 1H), 8.26 (s, 1H), 8.35 (s, 1H)

¹⁹F NMR (300 MHz, CD₃OD): −66.04

### Example 76

### 1-(2′-ethoxy-6′-(5-methyl-1,3,4-oxadiazol-2-yl)-4-(4-(trifluoromethyl)thiazol-2-yl)-3,4′-bipyridin-6-yl)-3-ethylurea

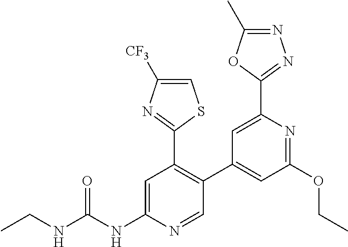

To a vial was charged methyl 6′-ethoxy-6-(3-ethylureido)-4-(4-(trifluoromethyl)thiazol-2-yl)-3,4′-bipyridine-2′-carboxylate (Intermediate 145, 200 mg, 0.404 mmol) with tetrahydrofuran (2 mL) and water (2 mL). Lithium hydroxide (100 mg) was added and the resulting mixture was stirred at room temperature overnight. The reaction mixture was diluted with water and methyl tert-butyl ether was added. Solid was observed between the layers and was collected and dried in a vacuum oven at 50° C. overnight.

The carboxylic salt (140 mg) was treated with acetic hydrazide (28 mg, 0.342 mmol) and phosphorus oxychloride (3 mL) then heated at 65° C. for 2 h. The excess phosphorus oxychloride was removed in vacuo and the residue was quenched by saturated sodium bicarbonate (30 mL). The product was extracted with ethyl acetate and tetrahydrofuran (3× each). The organic layers was combined and dried over sodium sulfate. After concentration, the crude mixture was triturated with ethanol (5 mL), and washed with methyl tert-butyl ether (3 mL) to give a white solid (45 mg, 30.4%).

MS (ESP): 520.2 (MH⁺) for C₂₂H₂₀F₃N₇O₃S

¹H NMR (300 MHz, CD₃OD): δ 1.22 (t, 3H), 1.39 (t, 3H), 3.31 (q, 2H), 4.45 (q, 2H), 6.86 (d, 1H), 7.32 (d, 1H), 7.79 (s, 1H), 8.26 (s, 1H), 8.35 (s, 1H)

¹⁹F NMR (300 MHz, CD₃OD): −66.04

### Example 77

### 1-(2′-ethoxy-6′-(5-oxo-4,5-dihydro-1,3,4-oxadiazol-2-yl)-4-(4-phenylthiazol-2-yl)-3,4′-bipyridin-6-yl)-3-ethylurea

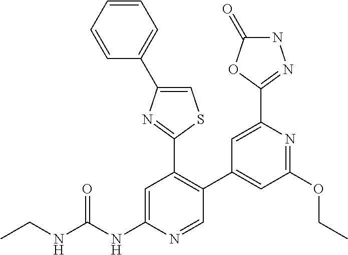

To a 100 mL round bottom flask was charged impure methyl 6′-ethoxy-6-(3-ethylureido)-4-(4-phenylthiazol-2-yl)-3,4′-bipyridine-2′-carboxylate (Intermediate 146, 400 mg) with ethanol (40 mL). Hydrazine monohydrate (6 mL) was added and the reaction mixture was heated to reflux for 2 h. After concentrating to dryness, the crude product was triturated with ethanol to remove Pd residue from previous step. The filtrate was concentrated under reduced pressure and dissolved in tetrahydrofuran (30 mL) with 1,1′-carbonyl diimidazole (230 mg, 1.42 mmol), and the mixture was stirred at room temperature overnight. After concentration under reduced pressure, the crude product was purified by Analogix (dichloromethane/methanol) to give an off-white solid (60 mg).

MS (ESP): 530.1 (MH⁺) for C₂₆H₂₃N₇O₄S

¹H NMR (300 MHz, CD₃OD): δ 1.23 (t, 3H), 1.38 (t, 3H), 3.35 (q, 2H), 4.46 (q, 2H), 6.90 (d, 1H), 7.32 (m, 3H), 7.42 (d, 1H), 7.70 (d, 1H), 7.73 (d, 1H), 7.81 (s, 1H), 7.91 (s, 1H), 8.31 (d, 1H)

### Example 78

### 1-(2′-ethoxy-6′-(5-methyl-1,3,4-oxadiazol-2-yl)-4-(4-phenylthiazol-2-yl)-3,4′-bipyridin-6-yl)-3-ethylurea

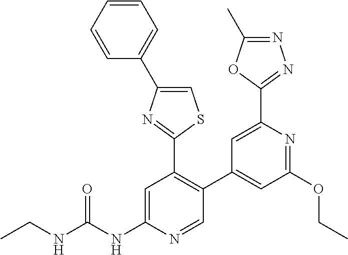

To a vial was charged methyl 6′-ethoxy-6-(3-ethylureido)-4-(4-phenylthiazol-2-yl)-3,4′-bipyridine-2′-carboxylate (Intermediate 146, 250 mg), tetrahydrofuran (30 mL) and water (30 mL). Lithium hydroxide (500 mg) was added and the resulting mixture was stirred at room temperature overnight. The reaction mixture was diluted with water and a cloudy mixture resulted. Filtration was applied to remove the solid. Methyl tert-butyl ether was added to the filtrate and a white solid precipitated which was collected as clean carboxylate salt (220 mg). The carboxylic salt (130 mg) was treated with acetic hydrazide (35 mg, 0.405 mmol) and phosphorus oxychloride (5 mL) then heated at 60° C. for 3 h. The solution was poured into cold saturated sodium bicarbonate (30 mL) in an ice bath and extracted with ethyl acetate (3×). The combined organic layers were dried over sodium sulfate and after concentration under reduced pressure, the crude material was purified by Analogix (dichloromethane/methanol) to give an off-white solid (60 mg, 43.3%).

MS (ESP): 528.0 (MH⁺) for C₂₇H₂₅N₇O₃S

¹H NMR (300 MHz, CD₃OD): δ 1.23 (t, 3H), 1.40 (d, 6H), 2.57 (s, 3H), 3.35 (q, 2H), 4.50 (q, 2H), 6.97 (d, 1H), 7.31 (m, 3H), 7.63 (d, 1H), 7.68 (d, 1H), 7.70 (d, 1H), 7.83 (s, 1H), 7.91 (s, 1H), 8.33 (d, 1H)

### Example 79

### 1-ethyl-3-(2′-isopropoxy-6′-(5-oxo-4,5-dihydro-1,3,4-oxadiazol-2-yl)-4-(4-(trifluoromethyl)thiazol-2-yl)-3,4′-bipyridin-6-yl)urea

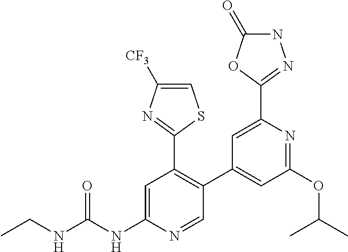

To a 100 mL round bottom flask was charged methyl 6-(3-ethylureido)-6′-isopropoxy-4-(4-(trifluoromethyl)thiazol-2-yl)-3,4′-bipyridine-2′-carboxylate (Intermediate 147, 450 mg) with ethanol (20 mL). Hydrazine monohydrate (6 mL) was added and the mixture heated to reflux for 1 h. After concentration under reduced pressure, the crude product was dried in a vacuum oven at 50° C. for 2 h. The residue was dissolved in tetrahydrofuran (30 mL), 1,1′-carbonyl diimidazole (0.53 g) was added and the mixture was stirred at room temperature for 3 h. Since starting material remained, another portion of 1,1′-carbonyl diimidazole (1 g) was added and the mixture was stirred for another 10 min. After concentration, the crude product was purified by prep. HPLC to give an off-white solid (130 mg)

MS (ESP): 536.1 (MH⁺) for C₂₂H₂₀F₃N₇O₄S

¹H NMR (300 MHz, CD₃OD): 1.22 (t, 3H), 1.36 (d, 6H), 3.35 (q, 2H), 5.46 (m, 1H), 6.89 (s, 1H), 7.41 (s, 1H), 7.92 (s, 1H), 8.33 (s, 1H), 8.36 (s, 1H)

¹⁹F NMR (300 MHz, CD₃OD): −65.92

### Example 80

### 1-ethyl-3-(2′-isopropoxy-6′-(5-methyl-1,3,4-oxadiazol-2-yl)-4-(4-(trifluoromethyl)thiazol-2-yl)-3,4′-bipyridin-6-yl)urea

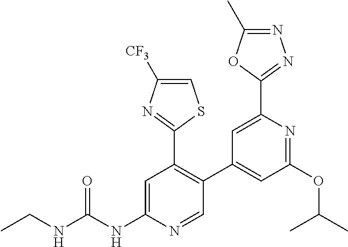

To a vial was charged methyl 6-(3-ethylureido)-6′-isopropoxy-4-(4-(trifluoromethyl)thiazol-2-yl)-3,4′-bipyridine-2′-carboxylate (Intermediate 147, 450 mg), tetrahydrofuran (30 mL) and water (30 mL). Lithium hydroxide (0.8 g) was added and the resulting mixture was stirred at room temperature for 1 h. The reaction mixture was filtered, the solid was washed with methyl tert-butyl ether and determined to be by-products. The aqueous layer was diluted with water and extracted with methyl tert-butyl ether twice. The aqueous was acidified with 6 N HCl to pH 2-3, and extracted with ethyl acetate (3×). The combined ethyl acetate layers were dried over sodium sulfate and dried in a vacuum oven at 50° C. for overnight to give a yellow solid (190 mg) as clean carboxylic acid.

The carboxylic acid (180 mg, 0.364 mmol) was treated with acetic hydrazide (48 mg, 0.581 mmol) and phosphorus oxychloride (3 mL) then heated at 65° C. for 2 h. The excess phosphorus oxychloride was removed in vacuo and the residue quenched by saturated sodium bicarbonate (30 mL). The resulting mixture was extracted with ethyl acetate (3×). The organic layers were combined and dried over sodium sulfate. After concentration under reduced pressure, the crude mixture was purified by Analogix (dichloromethane/methanol) to give a yellow solid (52 mg, 26.8%)

MS (ESP): 534.3 (MH⁺) for C₂₃H₂₂F₃N₇O₃S

¹H NMR (300 MHz, CD₃OD): 1.22 (t, 3H), 1.36 (d, 6H), 2.62 (s, 3H), 3.35 (q, 2H), 5.48 (m, 1H), 6.85 (d, 1H), 7.53 (d, 1H), 7.80 (s, 1H), 8.26 (d, 1H), 8.37 (d, 1H)

¹⁹F NMR (300 MHz, CD₃OD): −65.94

### Example 81

### 1-ethyl-3-(2′-isopropoxy-6′-(5-oxo-4,5-dihydro-1,3,4-oxadiazol-2-yl)-4-(4-phenylthiazol-2-yl)-3,4′-bipyridin-6-yl)urea

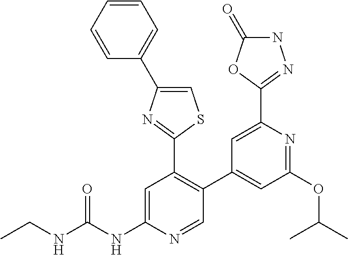

To a 100 mL round bottom flask was charged methyl 6-(3-ethylureido)-6′-isopropoxy-4-(4-phenylthiazol-2-yl)-3,4′-bipyridine-2′-carboxylate (Intermediate 148, 500 mg) with anhydrous ethanol (20 mL). Hydrazine monohydrate (6 mL) was added and the mixture was heated to reflux for 2 h. After concentration the crude product was dried in a vacuum oven at 60° C. overnight.

The crude hydrazide was dissolved in tetrahydrofuran (30 mL), 1,1′-carbonyl diimidazole (660 mg) added, and the mixture was stirred at room temperature for 1 h. After concentration under reduced pressure, the crude product was purified by prep. HPLC to give a yellow solid (100 mg).

MS (ESP): 544.2 (MH⁺) for C₂₇H₂₅N₇O₄S

¹H NMR (300 MHz, CD₃OD): δ 1.23 (t, 3H), 1.35 (d, 6H), 3.35 (q, 2H), 5.46 (m, 1H), 6.92 (d, 1H), 7.34 (m, 2H), 7.46 (d, 1H), 7.58 (d, 1H), 7.72 (d, 1H), 7.75 (d, 1H), 7.93 (s, 1H), 8.02 (s, 1H), 8.29 (s, 1H)

### Example 82

### 1-ethyl-3-(2′-isopropoxy-6′-(5-methyl-1,3,4-oxadiazol-2-yl)-4-(4-phenylthiazol-2-yl)-3,4′-bipyridin-6-yl)urea

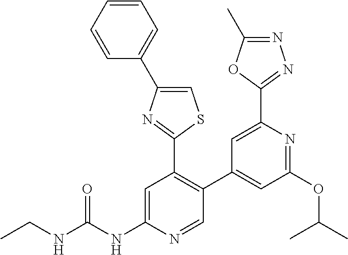

To a vial was charged methyl 6-(3-ethylureido)-6′-isopropoxy-4-(4-phenylthiazol-2-yl)-3,4′-bipyridine-2′-carboxylate (Intermediate 148, 0.66 g), tetrahydrofuran (30 mL) and water (30 mL). Lithium hydroxide (1 g) was added and the resulting mixture was stirred at room temperature overnight. The reaction mixture was diluted with water and a cloudy mixture resulted. Filtration was applied to remove the solid and the filtrate was extracted with methyl tert-butyl ether (3×). The aqueous layer was acidified by 6 N HCl to pH 2-3, and extracted with ethyl acetate (3×). The combined ethyl acetate layers were dried over sodium sulfate, to give solid carboxylic acid (100 mg). The carboxylic acid (100 mg, 0.199 mmol) was treated with acetic hydrazide (25 mg, 0.298 mmol) and phosphorus oxychloride (5 mL) then heated at 60° C. for 3 h. The solution was poured into cold saturated sodium bicarbonate (30 mL) in an ice bath and extracted with ethyl acetate (3×). The combined organic layers were dried over sodium sulfate. After concentration, the crude mixture was purified by Analogix (dichloromethane/methanol) to give a white solid (50 mg, 46.5%).

MS (ESP): 542.1 (MH⁺) for C₂₈H₂₇N₇O₃S

¹H NMR (300 MHz, CD₃OD): δ 1.23 (t, 3H), 1.35 (d, 6H), 2.57 (s, 3H), 3.35 (q, 2H), 5.48 (m, 1H), 6.91 (d, 1H), 7.29 (m, 3H), 7.60 (d, 1H), 7.66 (s, 1H), 7.67 (s, 1H), 7.82 (s, 1H), 7.91 (s, 1H), 8.33 (d, 1H).

### Example 83

### 1-(2′-(cyclopropylmethoxy)-6′-(5-oxo-4,5-dihydro-1,3,4-oxadiazol-2-yl)-4-(4-(trifluoromethyl)thiazol-2-yl)-3,4′-bipyridin-6-yl)-3-ethylurea

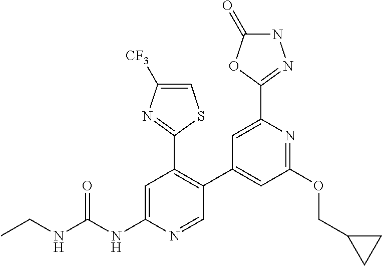

To a 100 mL round bottom flask was charged methyl 6′-(cyclopropylmethoxy)-6-(3-ethylureido)-4-(4-(trifluoromethyl)thiazol-2-yl)-3,4′-bipyridine-2′-carboxylate (Intermediate 149, 100 mg) with ethanol (20 mL). Hydrazine monohydrate (1.2 mL) was added and the mixture heated to reflux for 1 h. After concentrating the crude product was dried in a vacuum oven at 50° C. for 2 h.

The crude product was dissolved in anhydrous 1,4-dioxane (30 mL), 1,1′-carbonyl diimidazole (0.53 g) added and the mixture was stirred at room temperature for 3 h, starting material remained. Another portion of 1,1′-carbonyl diimidazole (0.4 g) was added, and the mixture was stirred for another 10 min. After concentration, the crude product was purified by Analogix (dichloromethane/methanol) to give an off-white solid (35 mg)

MS (ESP): 548.1 (MH⁺) for C₂₃H₂₀F₃N₇O₄S

¹H NMR (300 MHz, CD₃OD): 0.37-0.41 (m, 2H), 0.58-0.61 (m, 2H), 1.23 (t, 3H), 1.20-1.30 (m, 1H), 3.35 (q, 2H), 4.24 (d, 2H), 6.88 (d, 1H), 7.32 (d, 1H), 7.80 (s, 1H), 8.26 (s, 1H), 8.36 (s, 1H)

¹⁹F NMR (300 MHz, CD₃OD): −66.01

### Example 84

### 1-(2′-(cyclopropylmethoxy)-6′-(5-methyl-1,3,4-oxadiazol-2-yl)-4-(4-(trifluoromethyl)thiazol-2-yl)-3,4′-bipyridin-6-yl)-3-ethylurea

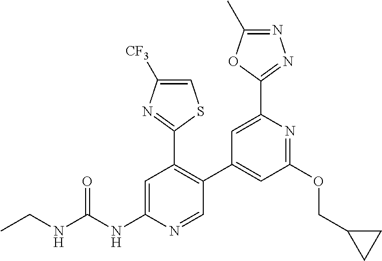

To a vial was charged methyl 6′-(cyclopropylmethoxy)-6-(3-ethylureido)-4-(4-(trifluoromethyl)thiazol-2-yl)-3,4′-bipyridine-2′-carboxylate (Intermediate 149), tetrahydrofuran (30 mL) and water (30 mL). Lithium hydroxide (0.5 g) was added and the resulting mixture was stirred at room temperature for 2 h. The mixture was extracted with ethyl acetate once then the aqueous layer acidified by 6 N HCl to pH 2-3. The resulting acidic solution was extracted with ethyl acetate (3×), the combined organic layers dried over sodium sulfate and concentrated. The crude carboxylic acid (450 mg) was treated with acetic hydrazide (70 mg, 0.945 mmol) and phosphorus oxychloride (4 mL). The mixture was heated at 65° C. for 3 hours, to give very impure product. The solution was poured into cold saturated sodium bicarbonate (30 mL) in an ice bath and extracted with ethyl acetate (3×). The organic layers was combined and dried over sodium sulfate. After concentration under reduced pressure, the crude mixture was purified by Analogix (dichloromethane/methanol) to give a light yellow solid (24 mg)

MS (ESP): 546.0 (MH⁺) for C₂₄H₂₂F₃N₇O₃S

¹H NMR (300 MHz, CD₃OD): 0.37-0.41 (m, 2H), 0.58-0.61 (m, 2H), 1.23 (t, 3H), 1.20-1.30 (m, 1H), 3.35 (q, 2H), 4.24 (d, 2H), 6.88 (d, 1H), 7.55 (d, 1H), 7.81 (s, 1H), 8.26 (d, 1H), 8.38 (d, 1H).

¹⁹F NMR (300 MHz, CD₃OD): −67.56

### Example 85

### 1-(2′-(cyclopropylmethoxy)-6′-(5-oxo-4,5-dihydro-1,3,4-oxadiazol-2-yl)-4-(4-phenylthiazol-2-yl)-3,4′-bipyridin-6-yl)-3-ethylurea

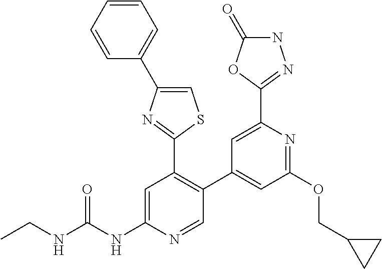

To a 100 mL round bottom flask was charged methyl 6′-(cyclopropylmethoxy)-6-(3-ethylureido)-4-(4-phenylthiazol-2-yl)-3,4′-bipyridine-2′-carboxylate (Intermediate 150, 600 mg) with anhydrous ethanol (20 mL). Hydrazine monohydrate (2.5 mL) was added and the mixture was heated to reflux for 2 h. After concentration under reduced pressure, the crude product was dried in a vacuum oven at 60° C. overnight.

The residue was dissolved in anhydrous 1,4-dioxane (30 mL), and 1,1′-carbonyl diimidazole (600 mg) was added, and the mixture was stirred at room temperature for 1 h. Another portion of 1,1′-carbonyl diimidazole (0.7 g) was added, and the reaction went to completion. After concentration under reduced pressure, the crude product was purified by prep. HPLC to give a white solid (15 mg).

MS (ESP): 556.2 (MH⁺) for C₂₈H₂₅N₇O₄S

¹H NMR (300 MHz, DMSO-d₆): δ 0.3-0.4 (m, 2H), 0.5-0.6 (m, 2H), 1.11 (t, 3H), 1.2-1.3 (m, 1H), 3.35 (q, 2H), 4.17 (d, 2H), 6.98 (s, 1H), 7.36-7.40 (m, 3H), 7.62 (s, 1H), 7.74-7.77 (m, 2H), 8.21 (s, 1H), 8.23 (d, 1H), 8.30 (s, 1H), 9.47 (s, 1H).

### Example 86

### 1-ethyl-3-(2′-morpholino-6′-(5-oxo-4,5-dihydro-1,3,4-oxadiazol-2-yl)-4-(4-(trifluoromethyl)thiazol-2-yl)-3,4′-bipyridin-6-yl)urea

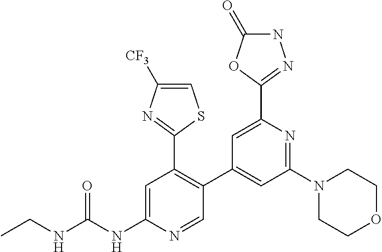

To a 100 mL round bottom flask was charged methyl 6-(3-ethylureido)-6′-morpholino-4-(4-(trifluoromethyl)thiazol-2-yl)-3,4′-bipyridine-2′-carboxylate (Intermediate 151, 150 mg, 0.28 mmol) with ethanol (20 mL). Hydrazine monohydrate (3 mL) was added and the mixture was heated to reflux for 0.5 h. While hot, the reaction mixture was filtered through a Celite pad to remove residual Pd catalyst from previous step. The filtrate was concentrated and dried in a vacuum oven at 50° C. for 2 h.

The crude was dissolved in tetrahydrofuran (30 mL), 1,1′-carbonyl diimidazole (0.2 g) added, and the mixture was stirred at room temperature for 2 h. Since starting material remained, another portion of 1,1′-carbonyl diimidazole (0.3 g) was added, and the mixture was stirred for another 1 h. After concentration under reduced pressure, the crude product was purified by Analogix, but the product still contained imidazole. The material was triturated by water to give an off-white solid (60 mg, 38.2%)

MS (ESP): 563.1 (MH⁺) for C₂₃H₂₁F₃N₈O₄S

¹H NMR (300 MHz, DMSO-d₆): 1.10 (t, 3H), 3.35 (q, 2H), 3.52-3.56 (m, 4H), 3.64-3.70 (m, 4H), 7.01 (d, 2H), 7.56 (m, 1H), 8.18 (s, 1H), 8.36 (s, 1H), 8.58 (d, 1H), 9.51 (s, 1H), 12.7 (m, 1H)

¹⁹F NMR (300 MHz, DMSO-d₆): −65.76

### Example 87

### 1-ethyl-3-(2′-morpholino-6′-(5-methyl-1,3,4-oxadiazol-2-yl)-4-(4-(trifluoromethyl)thiazol-2-yl)-3,4′-bipyridin-6-yl)urea

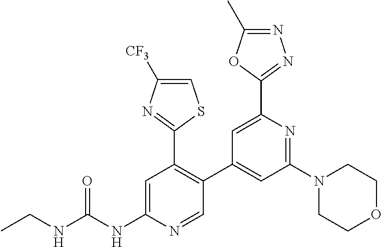

To a vial was charged methyl 6-(3-ethylureido)-6′-morpholino-4-(4-(trifluoromethyl)thiazol-2-yl)-3,4′-bipyridine-2′-carboxylate (Intermediate 151, 200 mg, 0.373 mmol), tetrahydrofuran (30 mL) and water (10 mL). Lithium hydroxide (0.5 g) was added, and the resulting mixture was stirred at room temperature for 1 h. The mixture was diluted with water and extracted with ethyl acetate once. The aqueous layer was acidified by 6 N HCl to pH 2-3, and extracted with ethyl acetate (3×). The combined organic layers were dried over sodium sulfate and after concentration dried in a vacuum oven at 50° C. overnight to give a yellow solid (120 mg, 61.5%).

The carboxylic acid (110 mg, 0.21 mmol) was treated with acetic hydrazide (26 mg, 0.32 mmol) and phosphorus oxychloride (3 mL) then heated at 60° C. for 2 h. The solution was poured into cold saturated sodium bicarbonate in an ice bath. The resulting mixture was extracted with ethyl acetate (3×). The organic layers were combined and dried over sodium sulfate. After concentration under reduced pressure, the crude mixture was purified by Analogix (dichloromethane/methanol) to give a yellow solid (41 mg, 34.9%)

MS (ESP): 561.3 (MH⁺) for C₂₄H₂₃F₃N₈O₃S

¹H NMR (300 MHz, CD₃OD): 1.22 (t, 3H), 2.61 (s, 3H), 3.34 (q, 2H), 3.60 (t, 4H), 3.77 (t, 4H), 6.92 (d, 1H), 7.30 (d, 1H), 7.82 (s, 1H), 8.25 (d, 1H), 8.36 (d, 1H)

¹⁹F NMR (300 MHz, CD₃OD): −65.79

### Example 88

### 1-ethyl-3-(2′-isopropoxy-6′-(5-oxo-4,5-dihydro-1,3,4-oxadiazol-2-yl)-4-(4-phenylthiazol-2-yl)-3,4′-bipyridin-6-yl)urea

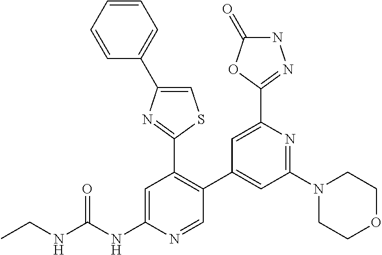

To a 100 mL round bottom flask was charged methyl 6-(3-ethylureido)-6′-morpholino-4-(4-phenylthiazol-2-yl)-3,4′-bipyridine-2′-carboxylate (Intermediate 152, 350 mg) with anhydrous ethanol (50 mL). Hydrazine monohydrate (2 mL) was added and the mixture was heated to reflux for 1 hour. After concentration under reduced pressure, the crude product was dried in a vacuum oven at 60° C. overnight.

The crude residue was dissolved in tetrahydrofuran (30 mL), 1,1′-carbonyl diimidazole (600 mg) added, and the mixture was stirred at room temperature for 1 h. After concentration under reduced pressure, the crude product was purified by Analogix (dichloromethane/methanol) to give a white solid (62 mg).

MS (ESP): 571.2 (MH⁺) for C₂₈H₂₆N₈O₄S

¹H NMR (300 MHz, CD₃OD): δ 1.11 (t, 3H), 3.22 (q, 2H), 3.50 (t, 4H), 3.65 (t, 4H), 6.97 (s, 1H), 7.10 (d, 2H), 7.36-7.45 (m, 3H), 7.67 (br, 1H), 7.83 (d, 1H), 7.85 (d, 1H), 8.22 (s, 1H), 8.24 (s, 1H), 8.29 (d, 1H), 9.48 (s, 1H)

### Example 89

### 1-ethyl-3-(2′-(5-methyl-1,3,4-oxadiazol-2-yl)-6′-morpholino-4-(4-phenylthiazol-2-yl)-3,4′-bipyridin-6-yl)urea

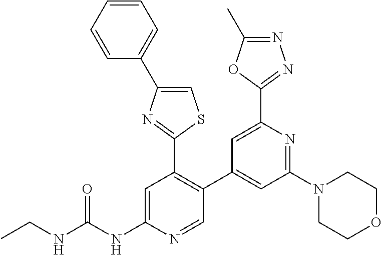

To a vial was charged methyl 6-(3-ethylureido)-6′-morpholino-4-(4-phenylthiazol-2-yl)-3,4′-bipyridine-2′-carboxylate (Intermediate 152, 0.34 g), tetrahydrofuran (30 mL) and water (30 mL). Lithium hydroxide (300 mg) was added, and the resulting mixture was stirred at room temperature overnight. The mixture was diluted with water and extracted with ethyl acetate once. The aqueous layer was then acidified to pH 2-3, and extracted with ethyl acetate (3×). The combined organic layers were dried over sodium sulfate, and after concentration, the resulting solid (acid) was used without further purification (100 mg).

The carboxylic acid (80 mg, 0.151 mmol) was treated with acetic hydrazide (25 mg, 0.305 mmol) and phosphorus oxychloride (3 mL) then heated at 60° C. for 4 h. The solution was poured into cold saturated sodium bicarbonate (30 mL) in an ice bath and extracted with ethyl acetate (3×). The combined organic layers were dried over sodium sulfate. After concentration, the crude mixture was purified by Analogix (dichloromethane/methanol) to give an off-white solid (25 mg).

MS (ESP): 569.1 (MH⁺) for C₂₉H₂₈N₈O₃S

¹H NMR (300 MHz, CD₃OD): δ ppm 1.23 (t, 3H), 2.57 (s, 3H), 3.35 (q, 2H), 3.57 (t, 4H), 3.71 (t, 4H), 6.94 (s, 1H), 7.33-7.39 (m, 4H), 7.75 (d, 1H), 7.78 (d, 1H), 7.82 (s, 1H), 7.90 (s, 1H), 8.35 (d, 1H)

### Example 90

### 1-ethyl-3-(2′-(1-methylpiperidin-4-yloxy)-6′-(5-oxo-4,5-dihydro-1,3,4-oxadiazol-2-yl)-4-(4-(trifluoromethyl)thiazol-2-yl)-3,4′-bipyridin-6-yl)urea

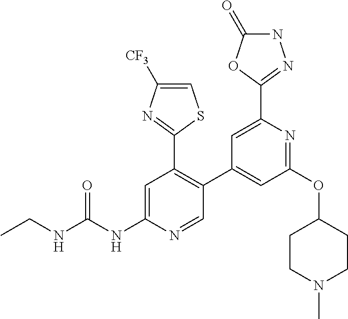

To a 100 mL round bottom flask was charged methyl 6-(3-ethylureido)-6′-(1-methylpiperidin-4-yloxy)-4-(4-(trifluoromethyl)thiazol-2-yl)-3,4′-bipyridine-2′-carboxylate (Intermediate 153, 100 mg, 0.213 mmol) with ethanol (20 mL). Hydrazine monohydrate (0.2 mL) was added and the mixture heated to reflux for overnight. The reaction was concentrated and dried in a vacuum oven at 40° C. for 2 h.

The crude product was dissolved in anhydrous tetrahydrofuran (10 mL), 1,1′-carbonyl diimidazole (80 mg) added, and the mixture was stirred at room temperature for 1 h. After concentration, the crude product was triturated with water to give an off-white solid (56 mg, 53.8% in two steps)

MS (ESP): 563.1 (MH⁺) for C₂₅H₂₅F₃N₈O₄S

¹H NMR (300 MHz, DMSO-d₆): 1.10 (t, 3H), 1.67-1.80 (m, 2H), 1.98-2.05 (m, 2H), 2.20 (s, 3H), 2.15-2.30 (m, 2H), 2.60-2.70 (m, 2H), 3.16-3.25 (m, 2H), 4.03 (br, 1H), 5.06 (m, 1H), 6.93 (d, 1H), 7.30 (d, 1H), 7.52 (s, 1H), 8.15 (d, 1H), 8.36 (s, 1H), 8.60 (d, 1H), 9.51 (s, 1H)

¹⁹F NMR (300 MHz, DMSO-d₆): −62.91

### Example 91

### 1-ethyl-3-(2′-(5-methyl-1,3,4-oxadiazol-2-yl)-6′-(1-methylpiperidin-4-yloxy)-4-(4-(trifluoromethyl)thiazol-2-yl)-3,4′-bipyridin-6-yl)urea

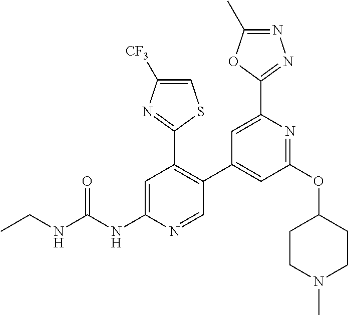

To a vial was charged methyl 6-(3-ethylureido)-6′-(1-methylpiperidin-4-yloxy)-4-(4-(trifluoromethyl)thiazol-2-yl)-3,4′-bipyridine-2′-carboxylate (Intermediate 153, 150 mg, 0.266 mmol), tetrahydrofuran (30 mL), sodium hydroxide (24 wt % in water, 0.5 mL) and the resulting mixture was stirred at room temperature for overnight. The mixture was concentrated to dryness and the crude salt used for the cyclization.

The carboxylic salt was treated with acetic hydrazide (37 mg, 0.449 mmol) and phosphorus oxychloride (4 mL) then heated at 65° C. for 1 h. The reaction went to completion based on LC, and the solution was poured into cold saturated sodium bicarbonate in an ice bath and extracted with ethanol/tetrahydrofuran (1:1) three times. The organic layers were combined and dried over sodium sulfate. After concentration, the crude mixture was purified by Analogix (dichloromethane/methanol) to give a light yellow solid (35 mg, 22.4%)

MS (ESP): 589.2 (MH⁺) for C₂₆H₂₇F₃N₈O₃S

¹H NMR (300 MHz, DMSO-d₆): 1.18 (t, 3H), 1.9-2.1 (br, 2H), 2.2-2.3 (br, 2H), 2.59 (s, 3H), 2.6-2.8 (br, 2H), 3.05 (q, 2H), 3.6-3.8 (br, 2H), 5.2-5.3 (br, 1H), 7.06 (s, 1H), 7.56 (br, 2H), 8.20 (s, 1H), 8.40 (s, 1H), 8.62 (s, 1H), 9.57 (s, 1H)

¹⁹F NMR (300 MHz, DMSO-d₆): −62.97

### Example 92

### 1-(2′-(2-(dimethylamino)ethoxy)-6′-(5-oxo-4,5-dihydro-1,3,4-oxadiazol-2-yl)-4-(4-(trifluoromethyl)thiazol-2-yl)-3,4′-bipyridin-6-yl)-3-ethylurea

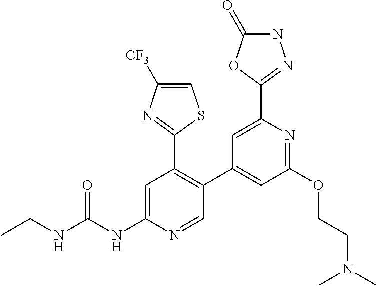

To a 100 mL round bottom flask was charged methyl 6′-(2-(dimethylamino)ethoxy)-6-(3-ethylureido)-4-(4-(trifluoromethyl)thiazol-2-yl)-3,4′-bipyridine-2′-carboxylate (Intermediate 154, 100 mg, 0.185 mmol) with of ethanol (20 mL). Hydrazine monohydrate (0.4 mL) was added and the mixture was heated to reflux for overnight. While hot, the reaction was filtered through a Celite pad to remove residual Pd catalyst and the filtrate was concentrated to dryness.

The crude product was dissolved in anhydrous tetrahydrofuran (10 mL), 1,1′-carbonyl diimidazole (110 mg) was added, and the mixture was stirred at room temperature for overnight. After concentration, the brown oily solid was triturated with water to give a light brown solid (50 mg, 47.6% in two steps)

MS (ESP): 565.2 (MH⁺) for C₂₃H₂₃F₃N₈O₄S

¹H NMR (300 MHz, DMSO-d₆): 1.10 (t, 3H), 2.22 (s, 6H), 2.65 (t, 2H), 3.20 (m, 2H), 4.42 (t, 2H), 6.99 (d, 1H), 7.32 (d, 1H), 7.54 (t, br, 1H), 7.62-7.70 (m, 1H), 8.15 (d, 1H), 8.36 (s, 1H), 8.60 (d, 1H), 9.53 (s, 1H)

¹⁹F NMR (DMSO-d₆): −62.90

### Example 93

### 1-(2′-(2-(dimethylamino)ethoxy)-6′-(5-methyl-1,3,4-oxadiazol-2-yl)-4-(4-(trifluoromethyl)thiazol-2-yl)-3,4′-bipyridin-6-yl)-3-ethylurea

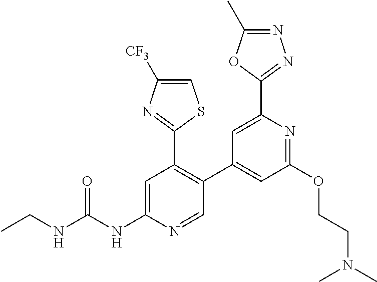

Methyl 6′-(2-(dimethylamino)ethoxy)-6-(3-ethylureido)-4-(4-(trifluoromethyl)thiazol-2-yl)-3,4′-bipyridine-2′-carboxylate (Intermediate 154, 500 mg) was treated with acetic hydrazide (100 mg, 1.21 mmol) and phosphorus oxychloride (5 mL) then heated at 65° C. for 1 h. After cooling the solution was poured into cold saturated sodium bicarbonate in an ice bath. The resulting mixture was extracted with ethanol/tetrahydrofuran (1:1) three times. The organic layers was combined and dried over sodium sulfate. After concentration, the crude mixture was purified by prep. HPLC

MS (ESP): 563.1 (MH⁺) for C₂₄H₂₅F₃N₈O₃S

### Example 94

### 1-ethyl-3-(5′-(5-methyl-1,3,4-oxadiazol-2-yl)-4-(4-phenylthiazol-2-yl)-3,3′-bipyridin-6-yl)urea

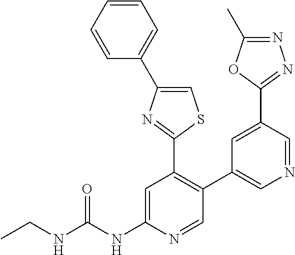

6-(3-ethylureido)-4-(4-phenylthiazol-2-yl)pyridin-3-ylboronic acid (0.100 g, 0.27 mmol, Intermediate 16), 2-(5-bromopyridin-3-yl)-5-methyl-1,3,4-oxadiazole (0.111 g, 0.46 mmol, Intermediate 418), cesium carbonate (0.150 g, 0.46 mmol), dicyclohexyl(2′,4′,6′-triisopropylbiphenyl-2-yl)phosphine (XPhos) (0.039 g, 0.08 mmol) and tris(dibenzylideneacetone)dipalladium(0) (0.025 g, 0.03 mmol) were combined in dioxane (2.00 mL)/water (0.50 mL) and heated to 100° C. The solution was cooled to room temperature and the reaction mixture was diluted with ethyl acetate and washed twice with water, once with saturated sodium bicarbonate and brine. The combined organic extracts were dried over magnesium sulfate, filtered and evaporated to a yellow solid. Isco column (0%-100% ethyl acetate/dichloromethane) afforded the desired compound as a white solid 0.106 g, 81% yield.

MS (ESP): 484 (M+H⁺) for C₂₃H₂₂F₃N₇O₄S.

¹H NMR (DMSO-d₆): δ 1.12 (t, 3H), 2.57 (s, 3H), 3.22 (q, 2H), 7.33 (q, 3H), 7.63 (m, 1H), 7.70 (d, 1H), 8.23 (s, 1H), 8.28 (s, 1H), 8.36 (s, 2H), 8.74 (s, 1H), 9.17 (s, 1H), 9.50 (s, 1H)

### Examples 95

The following compounds have been synthesized as described for Example 10 from the starting materials indicated in the table below.

| Ex | Compound | Data | SM |
|---|---|---|---|
| 95 | 1-ethyl-3-(2′-(5-oxo- 4,5-dihydro-1,3,4- oxadiazol-2-yl)-4-(4- phenylthiazol-2-yl)-3,4′- bipyridin-6-yl)urea | LC/MS (ES⁺)[(M + H)⁺]: 486 for C₂₄H₁₉N₇O₃S. ¹H NMR (300 MHz, d₆-DMSO): 1.1 (t, 3H), 3.2 (q, 2H), 7.54 (m, 2H), 7.68 (t, 1H), 7.9 (s, 1H), 8.20 (d, 2H), 8.33 (s, 1H), 8.7 (d, 1H), 9.52 (s, 1H), 12.78 (s, 1H). | Intermediate 155 |
| | 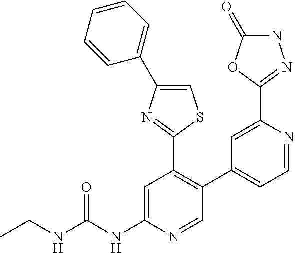 | | |

### Example 96

### 1-ethyl-3-(2′-(5-methyl-1,3,4-oxadiazol-2-yl)-4-(4-phenylthiazol-2-yl)-3,4′-bipyridin-6-yl)urea

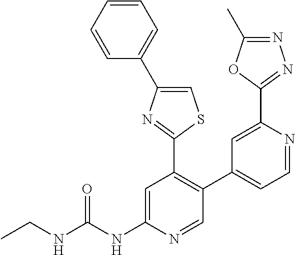

To a solution of 1-ethyl-3-(2′-(hydrazinecarbonyl)-4-(4-phenylthiazol-2-yl)-3,4′-bipyridin-6-yl)urea (0.036 g, 0.08 mmol, Intermediate 155) in THF (2.5 mL) and 1,1,1-trimethoxyethane (5 mL, 0.08 mmol) was added HCl (2.380 μL, 0.08 mmol) and the reaction was stirred at 120° C. DBU (0.118 mL, 0.78 mmol) was added and heating was continued. The reaction mixture was cooled to room temperature and concentrated to a red oil. Isco column (0%-10% methanol/dichloromethane) yielded pure product as a white solid 0.031 g, 82% yield.

MS (ESP): 484 (M+H⁺) for C₂₅H₂₁N₇O₂S.

¹H NMR (DMSO-d₆): δ 1.11 (t, 3H), 2.59 (s, 3H), 3.22 (q, 2H), 7.33 (q, 3H), 7.63 (dd, 4H), 8.11 (s, 1H), 8.22 (d, 1H), 8.36 (s, 1H), 8.75 (d, 1H), 9.53 (s, 1H).

### Example 97-98

The following compounds have been synthesized as described for Example 96 from the starting materials indicated in the table below.

| Ex | Compound | Data | SM |
|---|---|---|---|
| 97 | 1-ethyl-3-(5-(5-(5- methyl-1,3,4-oxadiazol- 2-yl)-4-(1-methyl-1H- 1,2,4-triazol-5- yl)thiazol-2-yl)-4-(4- phenylthiazol-2- yl)pyridin-2-yl)urea | LC/MS (ES⁺)[(M + H)⁺]: 571 for C₂₆H₂₂N₁₀O₂S₂. ¹H NMR (300 MHz, d₆-DMSO): 1.1 (t, 3H), 3.2 (q, 2H), 3.33 (s, 3H), 3.77 (s, 3H), 7.37 (m, 3H), 7.53 (m, 1H), 7.83 (d, 2H), 8.04 (s, 1H), 8.17 (s, 1H), 8.38 (s, 1H), 8.82 (s, 1H), 9.71 (s, 1H) | Intermediate 156 |
| | 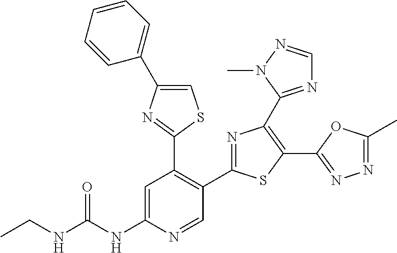 | | |
| 98 | 1-ethyl-3-(5-(5-(5- methyl-1,3,4-oxadiazol- 2-yl)-4-(pyrimidin-2- yl)thiazol-2-yl)-4-(4- phenylthiazol-2- yl)pyridin-2-yl)urea | LC/MS (ES⁺)[(M + H)⁺]: 568 for C₂₇H₂₁N₉O₂S₂. ¹H NMR (300 MHz, d₆-DMSO): 1.1 (t, 3H), 2.47 (s, 3H), 3.2 (q, 2H), 7.39 (m, 4H), 7.57 (m, 2H), 7.85 (d, 2H), 8.23 (s, 1H), 8.38 (s, 1H), 8.78 (s, 1H), 8.88 (d, 1H), 9.69 (s, 1H) | Intermediate 157 |
| | 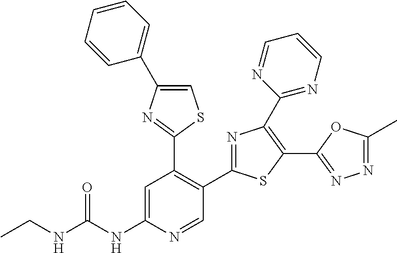 | | |

### Example 99

### 1-Ethyl-3-(2′-(2-(4-methylpiperazin-1-yl)ethoxy)-5′-(5-oxo-4,5-dihydro-1,3,4-oxadiazol-2-yl)-4-(4-(trifluoromethyl)thiazol-2-yl)-3,3′-bipyridin-6-yl)urea

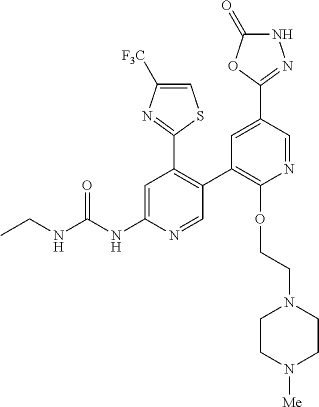

The 1-ethyl-3-(5′-(hydrazinecarbonyl)-2′-(2-(4-methylpiperazin-1-yl)ethoxy)-4-(4-(trifluoromethyl)thiazol-2-yl)-3,3′-bipyridin-6-yl)urea (Intermediate 165, 142 mg, 0.24 mmol) was dissolved in THF (2.4 mL) and cooled to 0° C. Diisopropylethylamine (46.0 μl, 0.26 mmol) was added dropwise, followed by the addition of 1,1′-carbonyldiimidazole (42.8 mg, 0.26 mmol). The ice bath was then removed and the mixture was stirred at RT for 3 h. The mixture was conc in vacuo and purified by silica gel chromatography (5-10% MeOH/CH₂Cl₂+1% NH₄OH) to give 16.6 mg (11%) of the title compound.

LC/MS (ES⁺)[(M+H)⁺]: 620 for C₂₆H₂₈F₃N₉O₄S

¹H NMR (DMSO-d₆): δ 9.47 (s, 1H); 8.65 (m, 1H); 8.54 (m, 1H); 8.25 (d, 2H); 8.14 (m, 1H); 7.61 (m, 1H); 4.15 (t, 2H); 3.21 (m, 2H); 2.18 (m, 10H); 2.07 (s, 3H); 1.10 (t, 3H).

### Example 100-103

The following compounds have been synthesized as described for Example 99 from the starting materials indicated in the table below.

| Ex | Compound | Data | SM |
|---|---|---|---|
| 100 | 1-(2′-(2- (Dimethylamino)ethoxy)- 5′-(5-oxo-4,5- dihydro-1,3,4- oxadiazol-2-yl)-4-(4- (trifluoromethyl)thiazol- 2-yl)-3,3′-bipyridin-6- yl)-3-ethylurea | LC/MS (ES⁺)[(M + H)⁺]: 565 for C₂₃H₂₃F₃N₈O₄S ¹H NMR (DMSO-d₆): δ 9.47 (s, 1H); 8.65 (m, 1H); 8.54 (s, 1H); 8.27 (s, 1H); 8.23 (s, 1H); 8.15 (m, 1H); 7.58 (m, 1H); 4.10 (t, 2H); 3.21 (m, 2H); 2.13 (t, 2H); 1.95 (s, 6H); 1.10 (t, 3H). | Intermediate 168 |
| | 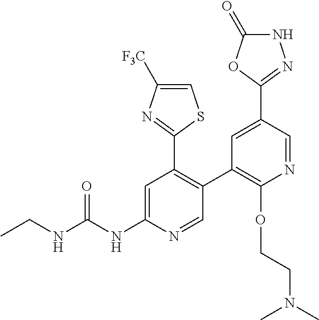 | | |
| 101 | 1-Ethyl-3-(2′-methoxy- 5′-(5-oxo-4,5-dihydro- 1,3,4-oxadiazol-2-yl)-4- (4- (trifluoromethyl)thiazol- 2-yl)-3,3′-bipyridin-6- yl)urea | LC/MS (ES⁺)[(M + H)⁺]: 508 for C₂₀H₁₆F₃N₇O₄S ¹H NMR (DMSO-d₆): δ 12.65 (br s, 1H); 9.47 (s, 1H); 8.66 (m, 1H); 8.55 (s, 1H); 8.29 (s, 1H); 8.20 (s, 1H); 8.13 (m, 1H); 7.58 (m, 1H); 3.59 (s, 3H); 3.21 (m, 2H); 1.10 (t, 3H). | Intermediate 170 |
| | 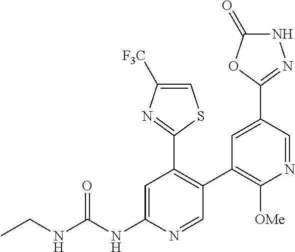 | | |
| 102 | 1-Ethyl-3-(2′-(2- morpholinoethoxy)-5′- (5-oxo-4,5-dihydro- 1,3,4-oxadiazol-2-yl)- 4-(4- (trifluoromethyl)thiazol- 2-yl)-3,3′-bipyridin-6- yl)urea | LC/MS (ES⁺)[(M + H)⁺]: 607 for C₂₅H₂₅F₃N₈O₅S ¹H NMR (DMSO-d₆): δ 12.67 (br s, 1H); 9.47 (s, 1H); 8.66 (m, 1H); 8.55 (s, 1H); 8.27 (s, 1H); 8.25 (s, 1H); 8.15 (m, 1H); 7.59 (m, 1H); 4.18 (m, 2H); 3.41 (m, 4H); 3.20 (m, 2H); 2.24 (m, 6H); 1.10 (t, 3H). | Intermediate 173 |
| | 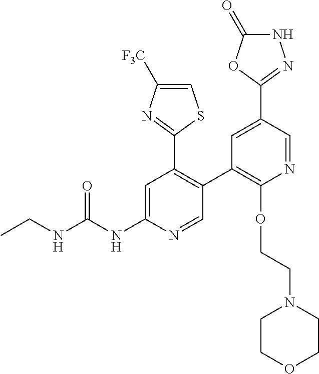 | | |
| 103 | 1-Ethyl-3-(2′-(5-methyl- 1,3,4-oxadiazol-2-yl)-4- (4-(pyridin-2-yl)thiazol- 2-yl)-3,4′-bipyridin-6- yl)urea | LC/MS (ES⁺)[(M + H)⁺]: 485 for C₂₄H₂₀N₈O₂S ¹H NMR (DMSO-d₆): δ 9.54 (s, 1H); 8.76 (d, 1H); 8.59 (m, 1H); 8.38 (s, 2H); 8.23 (s, 1H); 8.13 (s, 1H); 7.77 (m, 1H); 7.60 (m, 3H); 7.34 (m, 1H); 3.22 (m, 2H); 2.58 (s, 3H); 1.12 (t, 3H). | Intermediate 174 and Intermediate 176 |
| | 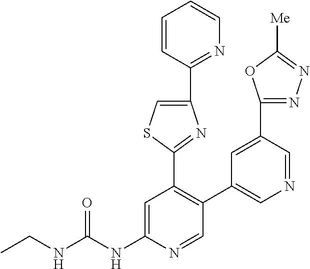 | | |

### Example 104

### 1-Ethyl-3-(5′-(5-methyl-1,3,4-oxadiazol-2-yl)-4-(4-(pyridin-2-yl)thiazol-2-yl)-3,3′-bipyridin-6-yl)urea

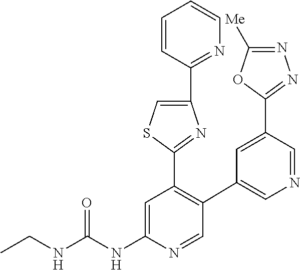

The 6-(3-ethylureido)-4-(4-(pyridin-2-yl)thiazol-2-yl)pyridin-3-ylboronic acid (Intermediate 174, 0.076 g, 0.21 mmol), 2-(5-bromopyridin-3-yl)-5-methyl-1,3,4-oxadiazole (Intermediate 418, 0.059 g, 0.25 mmol), tetrakis(triphenylphosphine)palladium (0) (0.024 g, 0.02 mmol) and cesium carbonate (0.101 g, 0.31 mmol) were placed in a microwave vessel. The vessel was degassed and purged with N₂ several times. Acetonitrile (2.5 ml) and water (0.625 ml) were added and the vessel was degassed and purged with N₂ again. The vessel was heated in the microwave at 100° C. for 2 h. The mixture was then conc in vacuo. Acetonitrile was added and the resultant precipitate was collected and washed with acetonitrile and water. Purification by silica gel chromatography (0-10% MeOH/CH₂Cl₂) gave 0.017 g (17%) of the title compound.

LC/MS (ES⁺)[(M+H)⁺]: 485 for C₂₄H₂₀N₈O₂S

¹H NMR (DMSO-d₆): δ 9.51 (s, 1H); 9.18 (d, 1H); 8.76 (d, 1H); 8.59 (m, 1H); 8.38 (m, 3H); 8.30 (s, 1H); 7.81 (m, 1H); 7.65 (m, 2H); 7.35 (m, 1H); 3.23 (m, 2H); 2.57 (s, 3H); 1.12 (t, 3H).

### Example 105

### 6′-(3-Ethylureido)-4′-(4-(pyridin-2-yl)thiazol-2-yl)-3,3′-bipyridine-5-sulfonamide

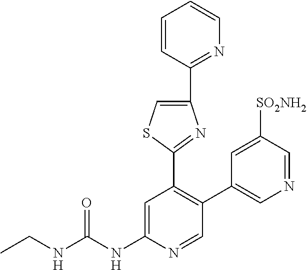

Following the procedure for Example 104, 6-(3-ethylureido)-4-(4-(pyridin-2-yl)thiazol-2-yl)pyridin-3-ylboronic acid (Intermediate 174, 0.076 g, 0.21 mmol) and 5-bromopyridine-3-sulfonamide (0.073 g, 0.31 mmol) were reacted in the microwave at 100° C. for 1 h. The mixture was then conc in vacuo. Acetonitrile was added and the resultant precipitate was collected and washed with acetonitrile and water to give 0.016 g (16%) of the title compound.

LC/MS (ES⁺)[(M+H)⁺]: 482 for C₂₁H₁₉N₇O₃S₂

¹H NMR (DMSO-d₆): δ 9.51 (s, 1H); 8.99 (m, 1H); 8.74 (m, 1H); 8.60 (m, 1H); 8.37 (s, 1H); 8.31 (m, 2H); 8.20 (m, 1H); 7.84 (m, 1H); 7.63 (m, 4H); 7.36 (m, 1H); 3.22 (m, 2H); 1.12 (t, 3H).

### Example 106

### 1-Ethyl-3-(2′-(5-oxo-4,5-dihydro-1,3,4-oxadiazol-2-yl)-4-(4-(pyridin-2-yl)thiazol-2-yl)-3,4′-bipyridin-6-yl)urea

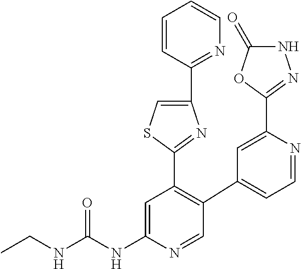

A solution of 1-ethyl-3-(2′-(hydrazinecarbonyl)-4-(4-(pyridin-2-yl)thiazol-2-yl)-3,4′-bipyridin-6-yl)urea (Intermediate 178, 56.4 mg, 0.12 mmol) in DMF (1 mL) was treated with diisopropylethylamine (0.03 mL, 0.18 mmol) and 1,1′-carbonyldiimidazole (29.8 mg, 0.18 mmol). The mixture was stirred at RT for 2 h. Methanol was added and the mixture was conc in vacuo. Purification by silica gel chromatography (0-10% MeOH/CH₂Cl₂) gave 15 mg (25%) of the title compound.

LC/MS (ES⁺)[(M+H)⁺]: 487 for C₂₃H₁₈N₈O₃S

¹H NMR (500 MHz, CDCl₃): δ 12.77 (br s, 1H); 9.52 (s, 1H); 8.73 (d, 1H); 8.61 (m, 1H); 8.39 (s, 1H); 8.36 (s, 1H); 8.24 (s, 1H); 7.92 (s, 1H); 7.82 (m, 1H); 7.59 (m, 3H); 7.36 (m, 1H); 3.23 (m, 2H); 1.13 (t, 3H).

### Example 107

### 1-Ethyl-3-(4-(1-isobutl-1H-pyrazol-4-yl)-5′-(5-oxo-4,5-dihydro-1,3,4-oxadiazol-2-yl)-3,3′-bipyridin-6-yl)urea

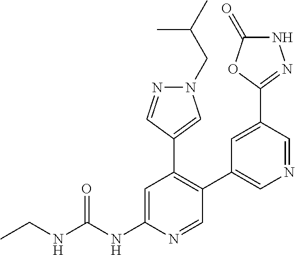

The 1-(4-chloro-5′-(5-oxo-4,5-dihydro-1,3,4-oxadiazol-2-yl)-3,3′-bipyridin-6-yl)-3-ethylurea (Intermediate 183, 0.065 g, 0.18 mmol) and cesium carbonate (0.117 g, 0.36 mmol) were placed in a microwave vessel. The vessel was degassed and purged with N₂. Tetrakis (triphenylphosphine)palladium (0) (0.021 g, 0.02 mmol) was added and the vessel was degassed and purged with N₂. The 1-isobutyl-4-(4,4,5,5-tetramethyl-1,3,2-dioxaborolan-2-yl)-1H-pyrazole (0.10 mL, 0.40 mmol) was added, followed by dioxane (1.6 mL) and water (0.4 mL). The vessel was degassed and purged with N₂ twice. The vessel was placed in the microwave for 2 h at 100° C. Purification by silica gel chromatography (0-10% MeOH/CH₂Cl₂) gave 0.017 g (21%) of the title compound.

LC/MS (ES⁺)[(M+H)⁺]: 449 for C₂₂H₂₄N₈O₃

¹H NMR (DMSO-d₆): δ 12.79 (s, 1H); 9.28 (s, 1H); 8.94 (m, 1H); 8.61 (m, 1H); 8.19 (s, 1H); 7.90 (m, 2H); 7.60 (s, 1H); 7.43 (s, 1H); 7.27 (s, 1H); 3.82 (d, 2H); 3.20 (m, 2H); 1.96 (m, 1H); 1.10 (t, 3H); 0.71 (s, 3H); 0.69 (s, 3H).

### Example 108

### 1-Ethyl-3-(4-(4-morpholinophenyl)-5′-(5-oxo-4,5-dihydro-1,3,4-oxadiazol-2-yl)-3,3′-bipyridin-6-yl)urea

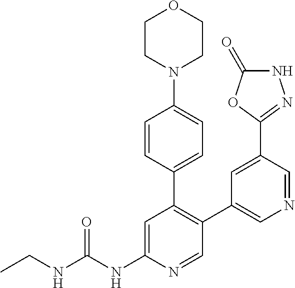

Diisopropylethylamine (0.058 mL, 0.33 mmol) was added to a solution of 1-ethyl-3-(5′-(hydrazinecarbonyl)-4-(4-morpholinophenyl)-3,3′-bipyridin-6-yl)urea (Intermediate 185, 0.102 g, 0.22 mmol) in DMF (2 mL). 1,1′-Carbonyldiimidazole (0.054 g, 0.33 mmol) was added in one portion and the resultant mixture was stirred at RT overnight. Purification by silica gel chromatography (0-10% MeOH/CH₂Cl₂) gave 0.066 g (62%) of the title compound.

LC/MS (ES⁺)[(M+H)⁺]: 488 for C₂₅H₂₅N₇O₄

¹H NMR (DMSO-d₆): δ 12.77 (s, 1H); 9.36 (s, 1H); 8.82 (m, 1H); 8.39 (m, 1H); 8.26 (s, 1H); 7.95 (m, 2H); 7.49 (s, 1H); 7.01 (m, 2H); 6.89 (m, 2H); 3.70 (m, 4H); 3.21 (m, 2H); 3.11 (m, 4H); 1.10 (t, 3H).

### Example 109

### 1-Ethyl-3-{5′-(5-oxo-4,5-dihydro-1,3,4-oxadiazol-2-yl)-2′-(piperidin-4-yloxy)-4-[4-(trifluoromethyl)-1,3-thiazol-2-yl]-3,3′-bipyridin-6-yl}urea

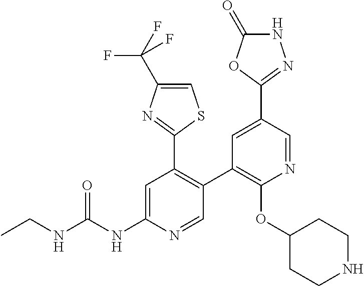

To a solution of tert-butyl 4-({6′-[(ethylcarbamoyl)amino]-5-(5-oxo-4,5-dihydro-1,3,4-oxadiazol-2-yl)-4′-[4-(trifluoromethyl)-1,3-thiazol-2-yl]-3,3′-bipyridin-2-yl}oxy)piperidine-1-carboxylate (Intermediate 191, 170 mg, 0.25 mmol) in dichloromethane (10 mL), trifluoroacetic acid (0.1 mL, 1.25 mmol) was added and stirred for 3 h at room temperature. Dichloromethane was evaporated from the reaction mixture, pH was adjusted to 8 with saturated sodium bicarbonate solution to obtain solid compound which was filtered and dried to afford 45 mg (31%) of 1-Ethyl-3-{5′-(5-oxo-4,5-dihydro-1,3,4-oxadiazol-2-yl)-2′-(piperidin-4-yloxy)-4-[4-(trifluoromethyl)-1,3-thiazol-2-yl]-3,3′-bipyridin-6-yl}urea.

¹H NMR (400 MHz, CDCl₃): δ 1.11 (m, 5H), 1.56 (br, 2H), 2.50 (m, 4H), 4.99 (m, 1H), 7.59 (m, 1H), 8.10 (m, 1H), 8.22-8.26 (m, 2H), 8.51-8.56 (m., 2H), 9.44 (s, 1H).

LC-MS: m/z 575.3 (M+H).

### Example 110

### 1-Ethyl-3-{5′-(5-methyl-1,3,4-oxadiazol-2-yl)-2′-(piperidin-4-yloxy)-4-[4-(trifluoromethyl)-1,3-thiazol-2-yl]-3,3′-bipyridin-6-yl}urea

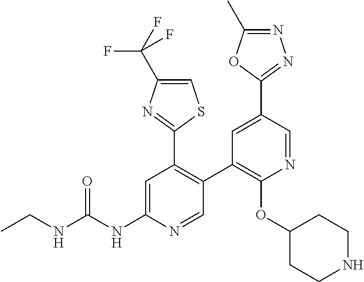

Tert-butyl 4-({6′-[(ethylcarbamoyl)amino]-5-(5-methyl-1,3,4-oxadiazol-2-yl)-4′-[4-(trifluoromethyl)-1,3-thiazol-2-yl]-3,3′-bipyridin-2-yl}oxy)piperidine-1-carboxylate (Intermediate 192, 150 mg, 0.22 mmol) was dissolved in dichloromethane (10 mL), trifluoroacetic acid (0.3 mL, 1.1 mM) was added and stirred for 3 h at room temperature. Dichloromethane was evaporated from the reaction mixture, pH was adjusted to 8 with saturated sodium bicarbonate solution to yield solid compound which was filtered and dried to afford 60 mg (47%) of 1-Ethyl-3-{5′-(5-methyl-1,3,4-oxadiazol-2-yl)-2′-(piperidin-4-yloxy)-4-[4-(trifluoromethyl)-1,3-thiazol-2-yl]-3,3′-bipyridin-6-yl}urea.

¹H NMR (400 MHz, CD₃OD): δ 1.34 (m, 5H), 1.76 (m, 3H), 2.62 (t, 3H), 2.72 (m, 5H), 4.28 (q, 2H), 5.21 (m, 1H), 8.21 (m, 1H), 8.29 (m, 2H), 8.58 (s, 1H), 8.85 (d, 1H)

LC-MS: m/z 576.2 (M+H)

### Example 111

### 3-({6′-[(ethylcarbamoyl)amino]-5-(5-oxo-4,5-dihydro-1,3,4-oxadiazol-2-yl)-4′-[4-(trifluoromethyl)-1,3-thiazol-2-yl]-3,3′-bipyridin-2-yl}oxy)propanoic acid

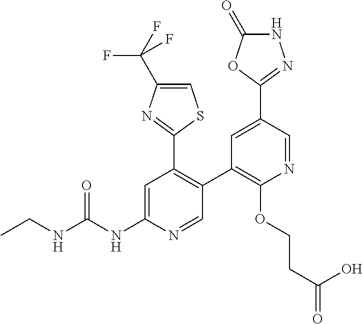

To a stirred solution of 3-({6′-[(ethylcarbamoyl)amino]-5-(hydrazinylcarbonyl)-4′-[4-(trifluoromethyl)-1,3-thiazol-2-yl]-3,3′-bipyridin-2-yl}oxy)propanoic acid (Intermediate 196, 240 mg, 0.44 mmol) in tetrahydrofuran (15 mL) which was cooled to 0° C., phosgene (66 mg, 0.66 mmol) (slowly added to the reaction mixture at 0° C. The reaction mixture was maintained at room temperature for 3 h. The solvent was distilled completely under reduced pressure to give crude product which was washed with diethyl ether and pentane and purified by reverse phase preparative HPLC to afford 45 mg (18%) of 3-({6′-[(ethylcarbamoyl)amino]-5-(5-oxo-4,5-dihydro-1,3,4-oxadiazol-2-yl)-4′-[4-(trifluoromethyl)-1,3-thiazol-2-yl]-3,3′-bipyridin-2-yl}oxy)propanoic acid.

¹H NMR (400 MHz, CD₃OD): δ 1.2.0-1.24 (t, 3H), 2.69 (br, 4H), 3.36 (m, 3H), 4.17 (br s, 2H), 7.789 (d, 1H), 7.98 (d, 1H), 8.19 (d, 1H), 8.26 (s, 1H), 8.39 (s, 2H).

LC-MS: m/z 566.3 (M+H).

### Example 112

### 1-Ethyl-3-{5′-(5-oxo-4,5-dihydro-1,3,4-oxadiazol-2-yl)-2′-(tetrahydro-2H-pyran-4-ylmethoxy)-4-[4-(trifluoromethyl)-1,3-thiazol-2-yl]-3,3′-bipyridin-6-yl}urea

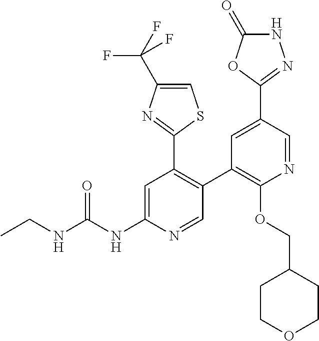

To a stirred solution of 1-Ethyl-3-{5′-(hydrazinylcarbonyl)-2′-(tetrahydro-2H-pyran-4-ylmethoxy)-4-[4-(trifluoromethyl)-1,3-thiazol-2-yl]-3,3′-bipyridin-6-yl}urea (Intermediate 199, 0.4 g, 0.70 mmol) in tetrahydrofuran (10 mL) which was cooled to 0° C., phosgene (0.1 g, 1.06 mmol) was slowly added to the reaction mixture at 0° C. The reaction mixture was maintained at room temperature for 3 h. The solvent was distilled off completely under reduced pressure to get crude compound which was washed with diethyl ether and pentane to afford 200 mg (48%) of 1-ethyl-3-{5′-(5-oxo-4,5-dihydro-1,3,4-oxadiazol-2-yl)-2′-(tetrahydro-2H-pyran-4-ylmethoxy)-4-[4-(trifluoromethyl)-1,3-thiazol-2-yl]-3,3′-bipyridin-6-yl}urea.

¹H NMR (400 MHz, DMSO-d₆): δ 0.91-1.23 (m, 4H), 1.55 (br, 1H), 1.76 (s, 1H), 1.90-1.92 (d, 1H), 2.08 (s, 1H), 3.08-3.14 (m, 2H), 3.19-3.22 (m, 2H), 3.67-3.69 (dd, 2H), 3.91-3.93 (d, 2H), 7.61 (br, 1H), 8.15 (d, 1H), 8.26-8.32 (m, 2H), 8.66-8.67 (d, 1H), 9.47-9.48 (d, 1H)

LC-MS: m/z 592.3 (M+2).

### Example 113

### 1-Ethyl-3-{5′-(5-methyl-1,3,4-oxadiazol-2-yl)-2′-(tetrahydro-2H-pyran-4-ylmethoxy)-4-[4-(trifluoromethyl)-1,3-thiazol-2-yl]-3,3′-bipyridin-6-yl}urea

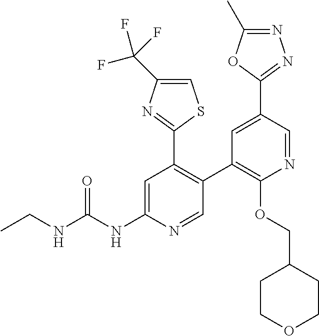

1-Ethyl-3-{5′-(hydrazinylcarbonyl)-2′-(tetrahydro-2H-pyran-4-ylmethoxy)-4-[4-(trifluoromethyl)-1,3-thiazol-2-yl]-3,3′-bipyridin-6-yl}urea (Intermediate 199, 400 mg, 1.7 mmol) was taken in triethylorthoacetate (5 mL) and the reaction mixture was heated to 120° C. for 12 h. The reaction mixture was cooled to room temperature, the solvent was distilled completely under reduced pressure to give crude product which was washed with diethyl ether and pentane to afford 150 mg 36.5% 1-ethyl-3-{5′-(5-methyl-1,3,4-oxadiazol-2-yl)-2′-(tetrahydro-2H-pyran-4-ylmethoxy)-4-[4-trifluoromethyl)-1,3-thiazol-2-yl]-3,3′-bipyridin-6-yl}urea as solid.

¹H NMR (400 MHz, DMSO-d₆): δ 1.09-1.13 (m, 2H), 1.31 (br, 2H), 1.37-1.56 (t, 3H), 1.69 (br, 1H), 2.63 (s, 3H), 3.23-3.29 (t, 2H), 3.84-3.87 (dd, 2H), 3.98-4.00 (d, 2H), 4.29-4.34 (q, 2H), 7.71 (s, 2H), 8.20 (d, 1H), 8.26 (s, 1H), 8.64 (s, 1H), 8.85 (d, 1H)

LC-MS: m/z 591 (M+2).

### Example 114-117

The following Examples were prepared according to the general procedure described below from the starting material indicated in the Table.

### General Procedure

A suspension of corresponding carboxylic acid (0.3 mmol), hydrazine acetate (0.9 mmol) in phosphorus oxychloride (2.5 mL) was heated at 70° C. for 2 h. The solution was then cooled and concentrated to dryness. A solution of saturated potassium carbonate was added to the crude and extracted with ethyl acetate (3×). The combined organic layers were washed with brine and dried over sodium sulfate. The solvent was removed under vacuum and the crude product was purified by Analogix using dichloromethane-methanol.

| Ex | Compound | Data | SM |
|---|---|---|---|
| 114 | 1-(2′-(1- (Dimethylamino)propan- 2-yloxy)-5′-(5- methyl-1,3,4- oxadiazol-2-yl)-4-(4- (trifluoromethyl)thiazol- 2-yl)-3,3′-bipyridin-6- yl)-3-ethylurea | MS (ESP): 577.2 (MH⁺) for C₂₅H₂₇F₃N₈O₃S ¹H NMR (300 MHz, CD₃OD): δ 0.88-0.91 (m, 3H), 1.24-1.28 (m, 3H), 1.57-1.59 (m, 6H), 2.63 (s, 3H), 2.72-2.87 (m, 2H), 3.44-347 (m, 2H), 4.84-4.87 (m, 2H), 5.65 (m, 1H), 7.91 (s, 1H), 8.26 (m, 1H) 8.32 (m, 1H), 8.36-8.37 (m, 1H), 8.90-8.91 (m, 1H). ¹⁹F NMR (300 MHz, CD₃OD): δ −65.81 | Intermediate 201 |
| | 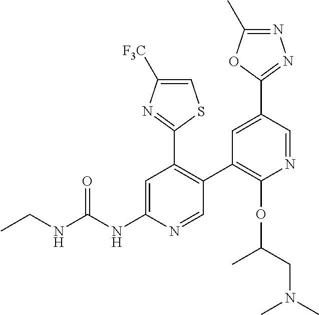 | | |
| 115 | 1-(2′-(2- (diethylamino)ethoxy)- 5′-(5-methyl-1,3,4- oxadiazol-2-yl)-4-(4- (trifluoromethyl)thiazol- 2-yl)-3,3′-bipyridin-6- yl)-3-ethylurea | MS (ESP): 591.2 (MH⁺) for C₂₆H₂₉F₃N₈O₃S ¹H NMR (300 MHz, CD₃OD): δ 0.91-0.94 (m, 6H), 1.21-1.22 (m, 3H), 2.47-2.50 (m, 4H), 2.52-2.55 (m, 2H) 2.62 (s, 3H), 3.34-3.36 (m, 2H), 4.25 (m, 2H), 7.91 (s, 1H), 8.22-8.26 (m, 1H), 8.30-8.31 (m, 2H), 8.86-8.86 (m, 1H). ¹⁹F NMR (300 MHz, CD₃OD) −65.83 | Intermediate 202 |
| | 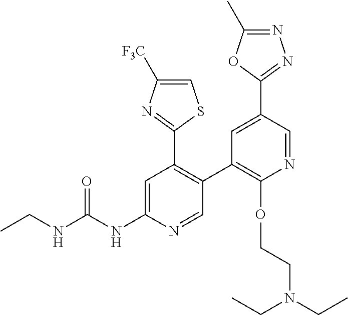 | | |
| 116 | 1-(2′-(2- (diisopropylamino)ethoxy)- 5′-(5-methyl-1,3,4- oxadiazol-2-yl)-4- (4(trifluoromethyl)thiazol- 2-yl)-3,3′-bipyridin- 6-yl)-3-ethylurea | MS (ESP): 619.2 (MH⁺) for C₂₈H₃₃F₃N₈O₃S ¹H NMR (300 MHz, CD₃OD): δ 1.15-1.19 (m, 3H), 1.33-1.39 (m, 12H), 2.62, (s, 3H), 3.24-3.28 (m, 4H), 3.77 (m, 2H), 3.82 (m, 2H), 7.94 (s, 1H), 8.26 (s, 1H), 8.32-8-34 (m, 2H), 8.89-8.90 (m, 1H) ¹⁹F NMR (300 MHz, CD₃OD): δ −65.798 | Intermediate 200 |
| | 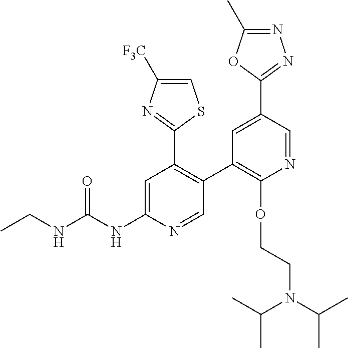 | | |
| 117 | 1-Ethyl-3-(5′-(5- methyl-1,3,4- oxadiazol-2-yl)-2′- (1,2,2,6,6- pentamethylpiperidin- 4-yloxy)-4-(4- (trifluoromethyl)thiazol- 2-yl)-3,3′-bipyridin-6- yl)urea | MS (ESP): 645.3 (MH⁺) for C₃₀H₃₅F₃N₈O₃S ¹H NMR (300 MHz, CD₃OD): δ 0.85-0.87 (m, 2H), 1.24-1.28 (m, 3H), 1.39 (s, 6H), 1.63 (s, 6H), 1.93 (m, 2H), 2.64 (s, 3H), 2.69 (m, 3H), 3.41-3.43 (m, 2H), 3.45-3.79 (m, 1H), 7.80 (m, 1H), 8.14-8.15 (m, 2H), 8.25 (m, 1H), 8.80-8.81 (m, 1H), 11.10 (m, 1H) ¹⁹F NMR (300 MHz, CDCl₃): δ −64.30 | Intermediate 203 |
| | 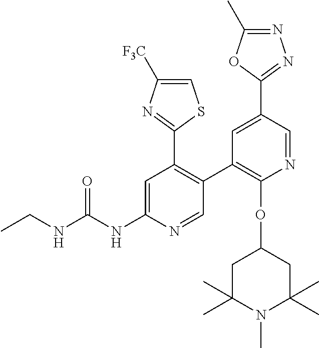 | | |

### Examples 118-121

The following Examples were prepared as described in the general procedure from the starting materials indicated in the Table.

### General Procedure

A suspension of the corresponding hydrazide (0.3 mmol) in anhydrous tetrahydrofuran (2 mL) was treated with triethyl amine (0.6 mmol) and 1,1′-carbonyl diimidazole (0.12 mmol). The reaction was stirred at room temperature for 12 h, concentrated to dryness and purified directly by Analogix using dichloromethane-methanol to give (˜50%) product as an off-white solid.

| Ex | Compound | Data | SM |
|---|---|---|---|
| 118 | 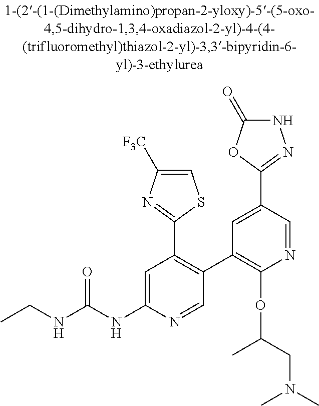 | MS (ESP): 579.3 (MH⁺) for C₂₄H₂₅F₃N₈O₄S ¹H NMR (300 MHz, DMSO-d₆): δ 0.81-0.87 (m, 3H), 1.08-1.13 (m, 3H), 1.97 (s. 6H), 3.18-3.25 (m, 4H), 5.10-5.16 (m, 1H), 7.02 (s, 1H), 7.57-7.60 (m, 1H), 8.15-8.19 (m, 2H), 8.26 (s, 1H), 8.54 (s, 1H), 8.62 (s, 1H), 9.45 (s, 1H). ¹⁹F NMR (300 MHz, DMSO-d₆): δ −63.007 | Intermediate 207 |
| 119 | 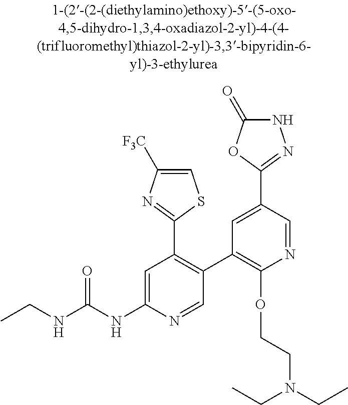 | MS (ESP): 593.1 (MH⁺) for C₂₅H₂₇F₃N₈O₄S H NMR (300 MHz, DMSO-d₆): δ 0.74-0.79 (m, 6H), 1.10-1.13 (m, 3H), 2.27-2.34 (m, 6H), 3.19-3.24 (m, 2H), 4.04-4.08 (m, 2H), 8.12- 8.14 (m, 1H), 8.23-8.26 (m, 2H), 8.54 (m, 1H), 8.63-8.64 (m, 1H), 9.44 (m, 1H) ¹⁹F NMR (300 MHz, DMSO-d₆): δ −102.2 | Intermediate 208 |
| 120 | 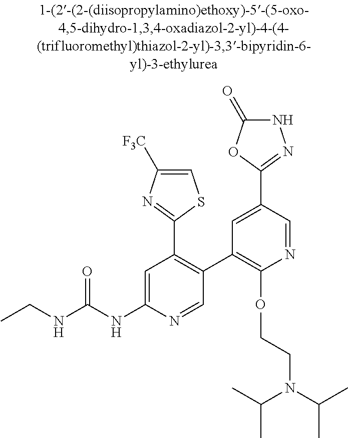 | MS (ESP): 621.3 (MH⁺) for C₂₇H₃₁F₃N₈O₄S ¹H NMR (300 MHz, CD₃OD): δ 0.80-0.85 (m, 12H), 0.93-0.95 (m, 3H), 2.19-2.27 (m, 1H), 2.31-2.33 (m, 1H), 2.73-2.81 (m, 2H), 3.21- 3.23 (m, 2H), 3.87-3.90 (m, 2H), 7.57 (m, 1H), 8.14-8.15 (m, 1H), 8.22-8.30 (m, 2H), 8.54-8.56 (m, 1H), 8.62-8.64 (m, 1H), 9.45 (m, 1H). ¹⁹F NMR (300 MHz, CD₃OD): δ - 63.07 | Intermediate 206 |
| 121 | 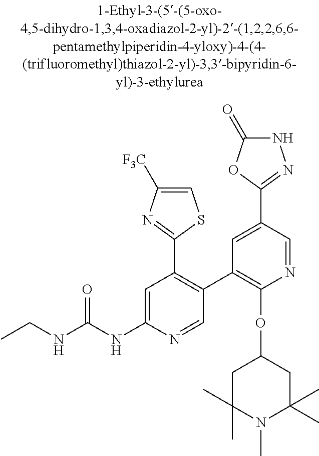 | MS (ESP): 647.1 (MH⁺) for C₂₉H₃₃F₃N₈O₄S ¹H NMR (300 MHz, CD₃OD): δ 0.87-0.89 (m, 3H), 1.19-1.22 (m, 6H), 1.24-1.28 (m, 12H), 1.30-1.41 (m, 4H), 2.74 (s, 3H), 3.31 (m, 3H), 7.91 (m, 1H), 8.18-8.19 (m, 1H), 8.25-8.26 (m, 2H), 8.69-8.70 (m, 1H). ¹⁹F NMR (300 MHz, CD₃OD): δ - 65.53 | Intermediate 209 |

### Example 122

### 1-(5′-(5-(1-amino-2-methylpropyl)-1,3,4-oxadiazol-2-yl)-2′-(2-(diethylamino)ethoxy)-4-(4-(trifluoromethyl)thiazol-2-yl)-3,3′-bipyridin-6-yl)-3-ethylurea hydrochloride

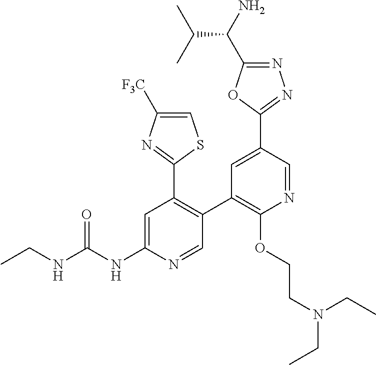

A suspension of (S)-tert-butyl 1-(5-(2-(2-(diethylamino)ethoxy)-6′-(3-ethylureido)-4′-(4-(trifluoromethyl)thiazol-2-yl)-3,3′-bipyridin-5-yl)-1,3,4-oxadiazol-2-yl)-2-methylpropylcarbamate (Intermediate 212, 0.2 mmol) in methanol (1 mL) was treated with HCl in 1,4-dioxane (4N, 2 mL) at room temperature for 6 h. The solution was then concentrated to dryness to give an off-white solid (80%).

MS (ESP): 648 (MH⁺) for C₂₉H₃₇ClF₃N₉O₃S

¹H NMR (300 MHz, CD₃OD): δ 1.06-1.28 (m, 15H), 2.48 (m, 2H), 2.65 (m, 1H), 3.10-3.11 (m, 4H), 3.31-3.32 (m, 2H), 3.73 (m, 1H), 4.76 (m, 2H), 8.01 (m, 1H), 8.35 (m, 1H), 8.40 (m, 1H), 8.44-8.45 (m, 1H), 9.01 (m, 1H)

¹⁹F NMR (300 MHz, CD₃OD): δ −65.81

### Example 123

### 1-(2′-(2-(Diisopropylamino)ethoxy)-5′-(5-methyl-1,3,4-oxadiazol-2-yl)-4-(4-(trifluoromethyl)thiazol-2-yl)-3,3′-bipyridin-6-yl)-3-propylurea

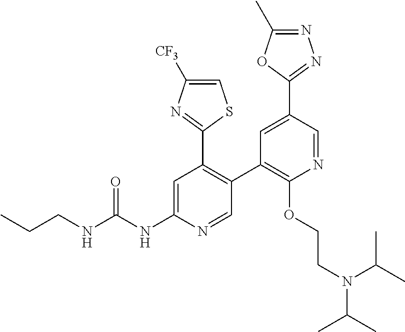

A suspension of 2-(2-(diisopropylamino)ethoxy)-6′-(3-propylureido)-4′-(4-(trifluoromethyl)thiazol-2-yl)-3,3′-bipyridine-5-carboxylic acid (Intermediate 220, 0.3 mmol), and hydrazine acetate (0.9 mmol) in phosphorus oxychloride (2.5 mL) was heated at 70° C. for 2 h. The solution was then cooled and concentrated to dryness. A solution of saturated potassium carbonate was added to the crude and product was extracted with ethyl acetate (3×). The combined organic layers were washed with brine and dried over sodium sulfate. All solvents were removed under vacuum and the crude was purified by Analogix using dichloromethane-methanol.

MS (ESP): 633.3 (M+H⁺) for C₂₉H₃₅F₃N₈O₃S

¹H NMR (300 MHz, CD₃OD): δ 0.95 (m, 12H), 0.99 (m, 3H), 1.64-1.66 (m, 2H), 2.35-2.40 (m, 2H) 2.62 9 (s, 3H), 2.88-2.92 (m, 2H), 3.27 (m, 2H), 4.02-4.06 (m, 2H), 7.84 (s, 1H), 8.27 (s, 1H), 8.29-8.30 (m, 2H), 8.83-8.84 (m, 1H).

¹⁹F NMR (300 MHz, CD₃OD): δ 65.92

### Example 124

### 1-(2′-(2-(Diisopropylamino)ethoxy)-5′-(5-oxo-4,5-dihydro-1,3,4-oxadiazol-2-yl)-4-(4-(trifluoromethyl)thiazol-2-yl)-3,3′-bipyridin-6-yl)-3-propylurea

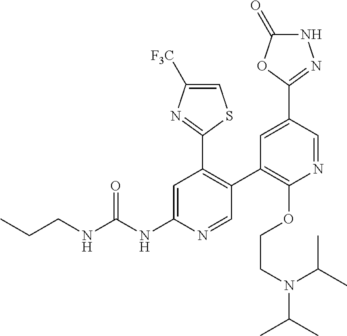

A suspension of 1-(2′-(2-(diisopropylamino)ethoxy)-5′-(5-oxo-4,5-dihydro-1,3,4-oxadiazol-2-yl)-4-(4-(trifluoromethyl)thiazol-2-yl)-3,3′-bipyridin-6-yl)-3-propylurea (Intermediate 221, 0.3 mmol) in anhydrous tetrahydrofuran (2 mL) was treated with triethyl amine (0.6 mmol) and 1,1′-carbonyl diimidazole (0.12 mmol). The reaction was stirred at room temperature for 12 h, concentrated to dryness and purified by Analogix chromatography using dichloromethane-methanol to give (50%) of an off-white solid.

MS (ESP): 635.1 (MH⁺) for C₂₈H₃₃F₃N₈O₄S

¹H NMR (300 MHz, DMSO-d₆): δ 0.81-0.88 (m, 12H), 0.90-0.93 (m, 3H), 1.51 (m, 2H), 2.16-2.18 (m, 2H), 2.82-2.84 (m, 2H), 3.14-3.18 (m, 2H), 3.84-3.86 (m, 2H), 7.01 (m, 1H), 7.63 (m, 1H), 8.02-8.02 (m, 1H) 8.21-8.25 (m, 2H), 8.48-8.51 (m, 2H), 9.43 (m, 1H)

¹⁹F NMR (300 MHz, DMSO-d₆): δ −62.97

### Examples 125-130

The following compounds have been synthesized as described for Example 21 from the starting materials indicated in the table below.

| Ex | Compound | Data | SM |
|---|---|---|---|
| 125 | 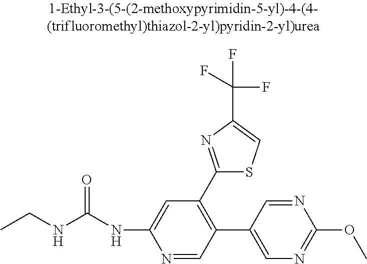 | MS (ESP): 425 (M + 1) for C₁₇H₁₅F₃N₆O₂S ¹H-NMR (DMSO-d₆) δ: 1.10 (t, 3H); 3.16-3.22 (m, 2H); 3.96 (s, 3H); 7.57 (brs, 1H); 8.24 (s, 1H); 8.32 (s, 1H); 8.58 (s, 3H); 9.47 (s, 1H). | 2-methoxy-5-(4,4,5,5- tetramethyl-1,3,2- dioxaborolan-2- yl)pyrimidine and Intermediate 3 |
| 126 | 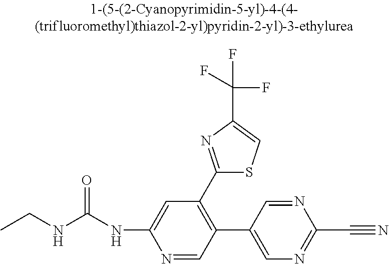 | MS (ESP): 420 (M + 1) for C₁₇H₁₂F₃N₇OS ¹H-NMR (DMSO-d₆) δ: 1.10 (t, 3H); 3.16-3.25 (m, 2H); 7.46 (brs, 1H); 8.25 (s, 1H); 8.44 (s, 1H); 8.65 (s, 1H); 9.00 (s, 2H); 9.60 (s, 1H). | 5-(4,4,5,5- tetramethyl-1,3,2- dioxaborolan-2- yl)pyrimidine-2- carbonitrile and Intermediate 3 |
| 127 | 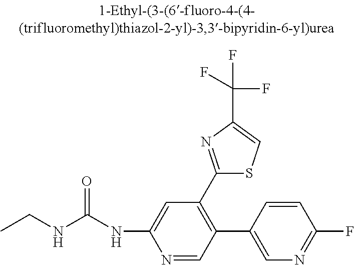 | MS (ESP): 412 (M + 1) for C₁₇H₁₃F₄N₅OS ¹H-NMR (DMSO-d₆) δ: 1.10 (t, 3H); 3.08-3.28 (m, 2H); 7.27 (dd, 1H); 7.57 (brs, 1H); 7.95 (td, 1H); 8.22 (s, 2H); 8.31 (s, 1H); 8.56 (s, 1H); 9.47 (s, 1H). | 6-fluoropyridin-3- ylboronic acid and Intermediate 3 |
| 128 | 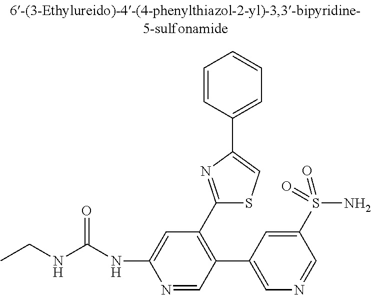 | MS (ESP): 481 (M + 1) for C₂₂H₂₀N₆O₃S₂ ¹H-NMR (DMSO-d₆) δ: 1.11 (t, 3H); 3.14-3.29 (m, 2H); 7.27-7.50 (m, 3H); 7.57 (brs, 1H); 7.66 (s, 2H); 7.73 (d, 2H); 8.18 (d, 1H); 8.25 (s, 1H); 8.31 (d, 2H); 8.73 (d, 1H); 9.00 (d, 1H); 9.50 (s, 1H). | Intermediate 161 and 5-bromopyridine-3- sulfonamide |
| 129 | 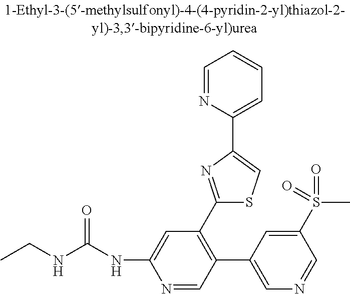 | MS (ESP): 481 (M + 1) for C₂₂H₂₀N₆O₃S₂ ¹H-NMR (DMSO-d₆) δ: 1.12 (t, 3H); 3.13-3.26 (m, 2H); 3.28 (s, 3H); 7.35 (dd, 1H); 7.51 (d, 1 H); 7.59 (brs, 1H); 7.82 (t, 1H); 8.28 (s, 1H); 8.33 (s, 1H); 8.38 (d, 2H); 8.59 (d, 1H); 8.87 (s, 1H); 9.09 (d, 1H); 9.52 (s, 1H). | Intermediate 15 and 5- (methylsulfonyl)pyridin- 3-ylboronic acid |
| 130 | 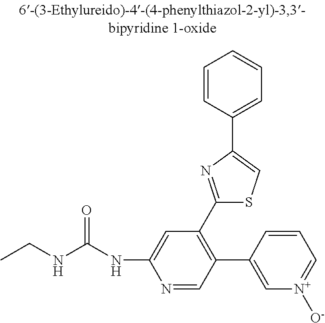 | MS (ESP): 418 (M + 1) for C₂₂H₁₉N₅O₂S ¹H-NMR (DMSO-d₆) δ: 1.11 (t, 3H); 3.17-3.29 (m, 2H); 7.21-7.53 (m, 5H); 7.62 (brs, 1H); 7.81 (d, 2H); 8.13-8.42 (m, 5H); 9.47 (s, 1H). | Intermediate 161 and 3-bromopyridine 1- oxide |

### Example 131

### 1-(5′-(2,4-Dioxo-1,2,3,4-tetrahydropyrimidin-5-yl)-4-(4-(trifluoromethyl)thiazol-2-yl)-3,3′-bipyridin-6-yl)-3-ethylurea

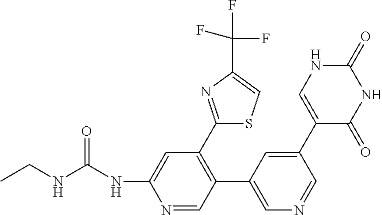

1-(5′-Bromo-4-(4-(trifluoromethyl)thiazol-2-yl)-3,3′-bipyridin-6-yl)-3-ethylurea (Example 21, 100 mg, 0.21 mmol), 2,4-dioxo-1,2,3,4-tetrahydropyrimidin-5-ylboronic acid (49.5 mg, 0.32 mmol), tris(dibenzylideneacetone)dipalladium(0) (19.39 mg, 0.02 mmol), 2-dicyclohexylphosphino-2′,4′,6′-tri-iso-propyl-1,1′-biphenyl (30.3 mg, 0.06 mmol) and sodium carbonate were taken in a round bottomed flask. It was degassed with nitrogen and 5 mL of dioxane:water (4:1) was added and degassed again. The resulting mixture was heated at 100° C. for 40 min, then the reaction mixture was filtered. The filtrate was concentrated under reduced pressure and the resulting residue was partitioned between water and 3% MeOH in dichloromethane. The layers were separated and the aqueous was back extracted with the solvent three times. The extracts were combined, washed with water and brine and dried over magnesium sulfate, then concentrated under reduced pressure and purified by reverse phase HPLC to give a white solid (62 mg).

MS (ESP): 504 (M+1) for C₂₁H₁₆F₃N₇O₃S

¹H-NMR (DMSO-d₆) δ: 1.10 (t, 3H); 3.01-3.48 (m, 2H); 7.57 (br s, 1H); 7.92 (d, 1H); 8.12 (s, 1H); 8.25 (s, 1H); 8.36 (s, 1H); 8.45 (d, 1H); 8.57 (s, 1H); 8.92 (d, 1H); 9.49 (s, 1H): 11.42 (brs, 2H).

### Examples 132-134

The following compounds have been synthesized as described for Example 131 from the starting materials indicated in the table below.

| Ex | Compound | Data | SM |
|---|---|---|---|
| 132 | 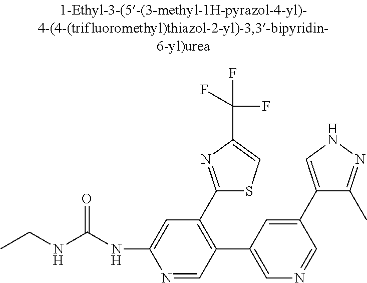 | MS (ESP): 474 (M + 1) for C₂₁H₁₈F₃N₇OS ¹H-NMR (DMSO-d₆) δ: 1.11 (t, 3H); 2.28 (s, 3H); 3.05-3.26 (m, 2H); 7.62 (br s, 1H); 7.79 (brs, 2H); 8.21 (s, 1H); 8.37 (d, 2H); 8.56 (s, 1H); 8.76 (d, 1H); 9.47 (s, 1H); 12.82 (brs, 1H). | Example 21 and 3- methyl-4-(4,4,5,5- tetramethyl-1,3,2- dioxaborolan-2-yl)- 1H-pyrazole |
| 133 | 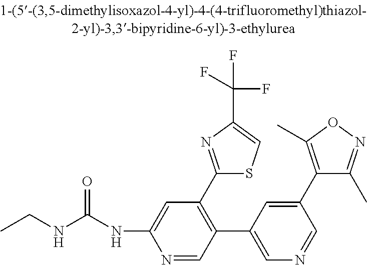 | MS (ESP): 489 (M + 1) for C₂₂H₁₉F₃N₆O₂S ¹H-NMR (DMSO-d₆) δ: 1.10 (t, 3H); 2.15 (s, 3H); 2.36 (s, 3H); 3.08-3.29 (m, 2H); 7.58 (br s, 1H); 7.80 (s, 1H); 8.21 (s, 1H); 8.37 (s, 1H); 8.55 (d, 1H); 8.57 (s, 1H); 8.67 (d, 1H); 9.48 (s, 1H). | Example 21 and 3,5- dimethyl-4-(4,4,5,5- tetramethyl-1,3,2- dioxaborolan-2- yl)isoxazole |
| 134 | 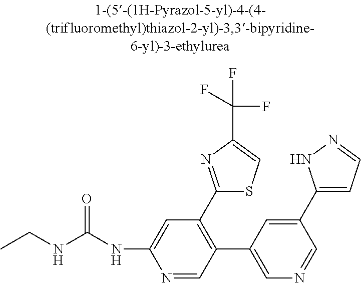 | MS (ESP): 460 (M + 1) for C₂₀H₁₆F₃N₇OS ¹H-NMR (DMSO-d₆) δ: 1.11 (t, 3H); 3.15-3.25 (m, 2H); 6.89 (d, 1H); 7.58 (br s, 1H); 7.83 (d, 1H); 8.27 (s, 1H); 8.30 (s, 1H); 8.39 (s, 1H); 8.50 (s, 1H); 8.55 (s, 1H); 9.14 (s, 1H); 9.51 (s, 1H). | Intermediate 12 and Intermediate 222 |

### Example 135

### 1-Ethyl-3-(5′-(5-oxo-4,5-dihydro-1H-1,2,4-triazol-3-yl)-4-(4-(trifluoromethyl)thiazol-2-yl)-3,3′-bipyridin-6-yl)urea

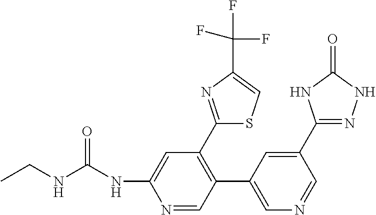

To a mixture of 1-(5′-(5-amino-1,3,4-oxadiazol-2-yl)-4-(4-(trifluoromethyl)thiazol-2-yl)-3,3′-bipyridin-6-yl)-3-ethylurea (Example 136, 70 mg, 0.15 mmol) in methanol (4 mL), potassium hydroxide (16.49 mg, 0.29 mmol) was added and heated at 70° C. for 20 h. The reaction mixture was cooled to room temperature, concentrated under reduced pressure, and the resulting residue was taken in conc. hydrochloric acid (3 mL) and heated at 80° C. for another 1 h. The reactiom mixture was cooled to room temperature and neutralized with 10 N sodium hydroxide solution. The solid that precipitated was collected by filtration, dried, and purified by reverse phase HPLC.

MS (ESP): 477 (M+1) for C₁₉H₁₅F₃N₈O₂S

¹H-NMR (DMSO-d₆) δ: 1.10 (t, 3H); 3.14-3.28 (m, 2H); 7.55 (brs, 1H); 8.10 (t, 1H); 8.25 (s, 1H); 8.36 (s, 1H); 8.56 (s, 2H); 9.00 (s, 1H); 9.51 (s, 1H); 11.89 (s, 1H); 12.17 (s, 1H)

### Example 136

### 1-(5′-(5-Amino-1,3,4-oxadiazol-2-yl)-4-(4-(trifluoromethyl)thiazol-2-yl)-3,3′-bipyridin-6-yl)-3-ethylurea

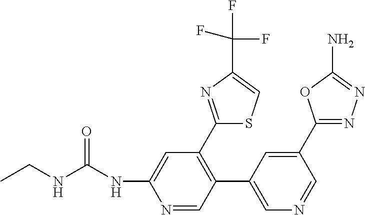

To a mixture of 1-ethyl-3-(5′-(hydrazinecarbonyl)-4-(4-(trifluoromethyl)thiazol-2-yl)-3,3′-bipyridin-6-yl)urea (Intermediate 9, 138 mg, 0.31 mmol) in 1,4-dioxane (3 mL), sodium bicarbonate (25.7 mg, 0.31 mmol) in water (1 mL) was added and the mixture was stirred for 5 min at room temperature. Cyanic bromide (0.122 mL, 0.37 mmol) (3M sol. in DCM) was added to the reactiom mixture and stirred at room temperature for 1 h. The product was precipitated with water, collected by filtration and dried to give a light yellow solid (101 mg).

MS (ESP): 477 (M+1) for C₁₉H₁₅F₃N₈O₂S

¹H-NMR (DMSO-d₆) δ: 1.11 (t, 3H); 3.14-3.28 (m, 2H); 7.43 (brs, 2H); 7.56 (brs, 1H); 8.08 (t, 1H); 8.24 (s, 1H); 8.38 (s, 1H); 8.56 (s, 1H); 8.61 (d, 1H); 9.00 (d, 1H); 9.51 (s, 1H).

### Example 137

### 1-Ethyl-3-(5-(5-(5-methyl-1,3,4-oxadiazol-2-yl)-4-(1-methyl-1H-1,2,4-triazol-5-yl)thiazol-2-yl)-4-(4-(trifluoromethyl)thiazol-2-yl)pyridin-2-yl)urea

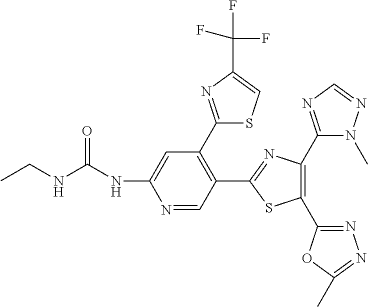

1-Ethyl-3-(5-(5-(hydrazinecarbonyl)-4-(1-methyl-1H-1,2,4-triazol-5-yl)thiazol-2-yl)-4-(4-(trifluoromethyl)thiazol-2-yl)pyridin-2-yl)urea (Intermediate 234, 70 mg, 0.13 mmol) was taken in 1,1,1-trimethoxyethane (2 mL, 0.13 mmol) and a drop of HCl was added to it. The mixture was refluxed at 120° C. for 25 min, then DMF (2 mL) and DBU (4-8 drops) were added and the mixture was refluxed for 20 h at 100° C. The reaction mixture was cooled to room temperature and water was added to precipitate the product. The product was collected via filtration and washed with 1:1 water and acetonitrile. The filtrate was extracted with ethyl acetate three times. The combined extracts were washed with water and brine, dried over magnesium sulfate and concentrated. The crude was combined with the precipitated product and purified by normal phase chromatography (2% MeOH in DCM to 6% MeOH in DCM). The fractions containing the product were combined and concentrated to give off-white solid (20 mg).

MS (ESP): 563 (M+1) for C₂₁H₁₇F₃N₁₀O₂S₂

¹H-NMR (DMSO-d₆) δ: 1.11 (t, 3H); 2.50 (s, 3H); 3.13-3.27 (m, 2H); 3.80 (s, 3H); 7.48 (brs, 1H); 8.05 (s, 1H); 8.11 (s, 1H); 8.74 (s, 1H); 8.85 (s, 1H); 9.76 (s, 1H).

### Example 138

### 1-Ethyl-3-(5′-(5-methyl-1,3,4-oxadiazol-2-yl)-4-(5-methyl-4-(trifluoromethyl)thiazol-2-yl)-3,3′-bipyridin-6-yl)urea

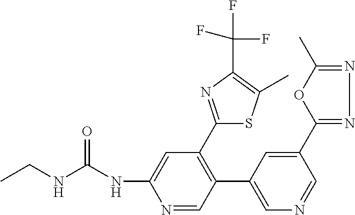

The title compound was synthesized by a method analogous to the synthesis of Example 137 starting with 1-ethyl-3-(5′-(hydrazinecarbonyl)-4-(5-methyl-4-(trifluoromethyl)thiazol-2-yl)-3,3′-bipyridin-6-yl)urea Intermediate 237 and 1,1,1-trimethoxyethane.

MS (ESP): 490 (M+1) for C₂₁H₁₈F₃N₇O₂S

¹H-NMR (DMSO-d₆) δ: 1.11 (t, 3H); 2.52 (s, 3H); 2.60 (s, 3H); 3.09-3.29 (m, 2H); 7.58 (br s, 1H); 8.18 (s, 1H); 8.31 (s, 1H); 8.36 (s, 1H); 8.70 (d, 1H); 9.17 (d, 1H); 9.51 (s, 1H).

### Examples 139-142

The following compounds have been synthesized as described for Example 21 from the starting materials indicated in the table below.

| Ex | Compound | Data | SM |
|---|---|---|---|
| 139 | 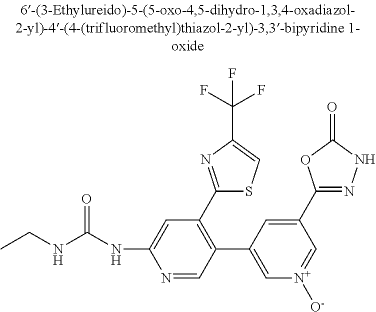 | MS (ESP): 494 (M + 1) for C₁₉H₁₄F₃N₇O₄S ¹H-NMR (DMSO-d₆) δ: 1.10 (t, 3H); 3.15-3.24 (m, 2H); 7.53 (brs, 1H); 7.61 (s, 1H); 8.22 (s, 1H); 8.38 (s, 1H); 8.49 (s, 1H); 8.53 (s, 1H); 8.64 (s, 1H); 9.54 (s, 1H); 12.95 (s, 1H) | 3-bromo-5-(5-oxo- 4,5-dihydro-1,3,4- oxadiazol-2- yl)pyridine 1-oxide (Intermediate 465) and Intermediate 12 |
| 140 | 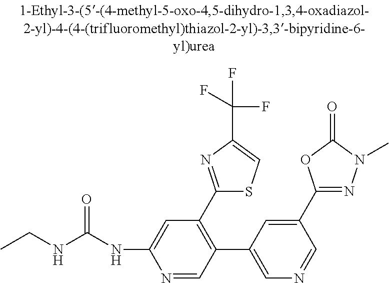 | MS (ESP): 492 (M + 1) for C₂₀H₁₆F₃N₇O₃S ¹H-NMR (DMSO-d₆) δ: 1.11 (t, 3H); 3.12-3.29 (m, 2H); 3.42 (s, 3H); 7.56 (brs, 1H); 8.13 (d, 1H); 8.23 (s, 1H); 8.38 (s, 1H); 8.59 (s, 1H); 8.66 (d, 1H); 8.99 (d, 1H); 9.52 (s, 1H) | Intermediate 12 and Intermediate 246 |
| 141 | 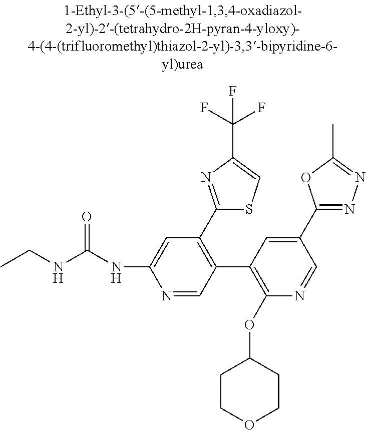 | MS (ESP): 576 (M + 1) for C₂₅H₂₄F₃N₇O₄S ¹H-NMR (DMSO-d₆) δ: 1.05-1.15 (m, 2H); 1.11 (t, 3H); 1.64 (br s, 2H); 2.59 (s, 3H); 3.09-3.27 (m, 2H); 3.30-3.36 (m, 2H); 3.38-3.71 (m, 2H); 4.81-5.28 (m, 1H); 7.59 (brs, 1H); 8.22 (s, 1H); 8.31 (s, 1H); 8.33 (d, 1H); 8.54 (s, 1H); 8.82 (d, 1H); 9.50 (s, 1H). | Intermediate 12 and Intermediate 484 |
| 142 | 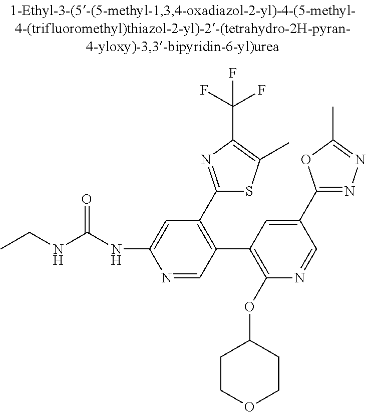 | MS (ESP): 590 (M + 1) for C₂₆H₂₆F₃N₇O₄S ¹H-NMR (DMSO-d₆) δ: 1.05-1.16 (m, 2 H); 1.11 (t, 3H); 1.65 (brs, 2H); 2.52 (s, 3H); 2.59 (s, 3H); 3.09-3.27 (m, 2H); 3.32-3.36 (m, 2H); 3.38-3.67 (m, 2H); 4.82-5.38 (m, 1H); 7.61 (brs, 1H); 8.16 (s, 1H); 8.27 (s, 1 H); 8.29 (d, 1H); 8.81 (d, 1H); 9.48 (s, 1H). | Intermediate 245 and Intermediate 484 |

### Examples 143-151

The following compounds have been synthesized as described for Example 6 from the starting materials indicated in the table below.

| Ex | Compound | Data | SM |
|---|---|---|---|
| 143 | 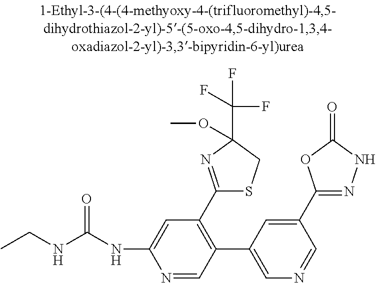 | MS (ESP): 510 (M + 1) for C₂₀H₁₈F₃N₇O₄S ¹H-NMR (DMSO-d₆) δ: 1.11 (t, 3H); 3.11 (s, 3H); 3.10-3.29 (m, 2H); 3.76 (s, 2H); 7.42 (brs, 1H); 8.05 (s, 1H); 8.09 (s, 1H); 8.41 (s, 1H); 8.71 (s, 1 H); 8.96 (s, 1H); 9.53 (s, 1H); 12.82 (s, 1H). | Intermediate 235 |
| 144 | 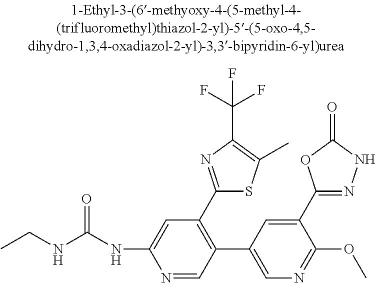 | MS (ESP): 522 (M + 1) for C₂₁H₁₈F₃N₇O₄S ¹H-NMR (DMSO-d₆) δ: 1.10 (t, 3H); 2.53 (s, 3H); 3.15-3.24 (m, 2H); 4.03 (s, 3H); 7.62 (brs, 1H); 8.03 (d, 1H); 8.18 (s, 1H); 8.29 (s, 1H); 8.32 (d, 1H); 9.45 (s, 1H); 12.68 (s, 1H) | Intermediate 236 |
| 145 | 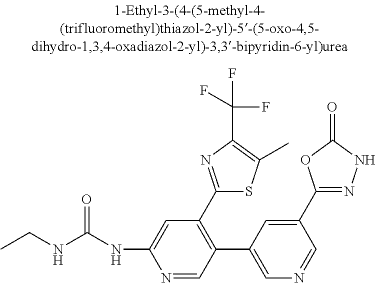 | MS (ESP): 492 (M + 1) for C₂₀H₁₆F₃N₇O₃S ¹H-NMR (DMSO-d₆) δ: 1.11 (t, 3H); 2.51 (s, 3H); 3.15-3.24 (m, 2H); 7.58 (brs, 1H); 8.16 (s, 1H); 8.18 (s, 1H); 8.34 (s, 1H); 8.65 (s, 1H); 9.00 (s, 1H); 9.49 (s, 1H); 12.78 (s, 1H) | Intermediate 237 |
| 146 | 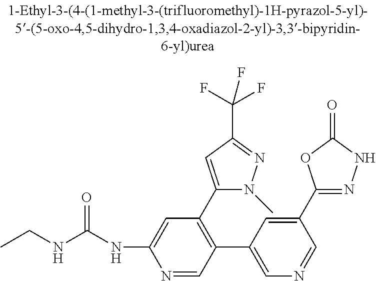 | MS (ESP): 475 (M + 1) for C₂₀H₁₇F₃N₈O₃ ¹H-NMR (DMSO-d₆) δ: 1.11 (t, 3H); 3.11-3.28 (m, 2H); 3.44 (s, 3H); 6.88 (s, 1H); 7.63 (brs, 1H); 7.70 (s, 1H); 7.93 (t, 1H); 8.52 (s, 1H); 8.53 (s, 1H); 8.89 (d, 1H); 9.55 (s, 1H); 12.80 (s, 1H) | Intermediate 238 |
| 147 | 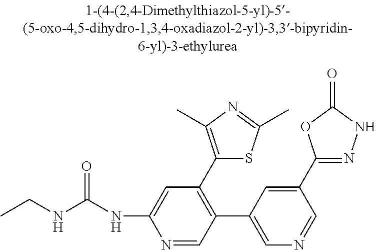 | MS (ESP): 438 (M + 1) for C₂₀H₁₉N₇O₃S ¹H-NMR (DMSO-d₆) δ: 1.10 (t, 3H); 1.89 (s, 3H); 2.57 (s, 3H); 3.11-3.28 (m, 2H); 7.62 (s, 1H); 7.73 (brs, 1H); 7.97 (t, 1H); 8.39 (s, 1H); 8.51 (s, 1H); 8.88 (d, 1H); 9.45 (s, 1H); 12.79 (s, 1H) | Intermediate 239 |
| 148 | 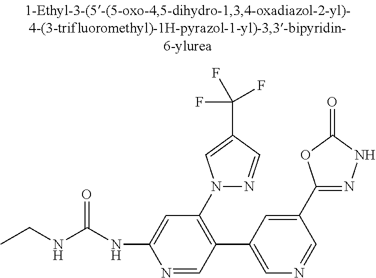 | MS (ESP): 461 (M + 1) for C₁₉H₁₅F₃N₈O₃ ¹H-NMR (DMSO-d₆) δ: 1.11 (t, 3H); 3.11-3.28 (m, 2H); 6.94 (d, 1H); 7.41 (t, 1H); 7.76 (t, 1H); 7.95 (s, 1H); 8.09 (s, 1H); 8.43 (d, 1H); 8.53 (s, 1H); 8.90 (d, 1H); 9.58 (s, 1H); 12.78 (s, 1H) | Intermediate 240 |
| 149 | 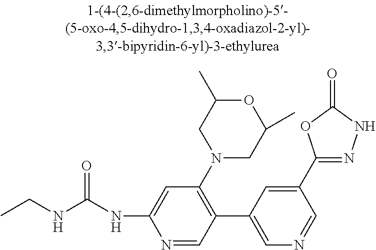 | MS (ESP): 440 (M + 1) for C₂₁H₂₅N₇O₄ ¹H-NMR (DMSO-d₆) δ: 0.96 (d, 6H); 1.09 (t, 3H); 2.28 (t, 2H); 2.95 (d, 2H); 3.11-3.26 (m, 2H); 3.51 (brs, 2H); 7.13 (s, 1H); 8.01 (s, 2H); 8.38 (t, 1H); 8.91 (t, 2 H); 9.11 (s, 1H); 12.72 (s, 1H) | Intermediate 241 |
| 150 | 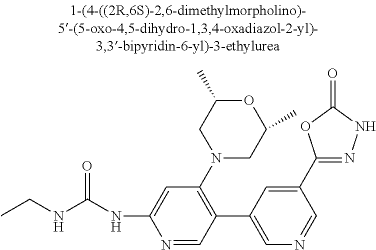 | MS (ESP): 440 (M + 1) for C₂₁H₂₅N₇O₄ ¹H-NMR (DMSO-d₆) δ: 0.96 (d, 6H); 1.09 (t, 3H); 2.27 (t, 2H); 2.95 (d, 2H); 3.11-3.26 (m, 2H); 3.51 (brs 2H); 7.13 (s, 1H); 8.01 (s, 2H); 8.38 (s, 1H); 8.91 (s, 2 H); 9.13 (s, 1H); 12.80 (s, 1H) | Intermediate 242 |
| 151 | 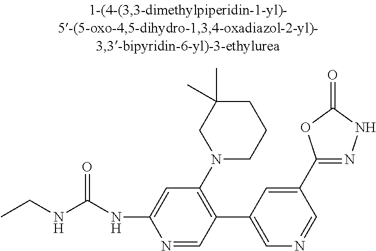 | MS (ESP): 438 (M + 1) for C₂₂H₂₇N₇O₃ ¹H-NMR (DMSO-d₆) δ: 0.89 (s, 6H); 1.09 (t, 3H); 1.15-1.51 (m, 4H); 2.54-2.79 (m, 4H); 3.09-3.26 (m, 2H); 7.13 (s, 1H); 7.95 (s, 1H); 8.09 (brs, 1H); 8.33 (s, 1H); 8.86 (d, 1 H); 8.92 (d, 1H); 9.11 (s, 1H); 12.80 (s, 1H) | Intermediate 243 |

### Example 152

### 1-(5′-(5-(3-(Dimethylamino)propylamino)-1,3,4-oxadiazol-2-yl)-4-(4-(trifluoromethyl)thiazol-2-yl)-3,3′-bipyridin-6-yl)-3-ethylurea

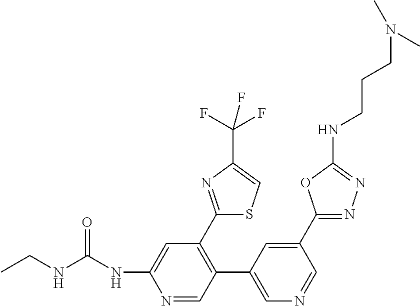

To a solution 1-ethyl-3-(5′-(5-oxo-4,5-dihydro-1,3,4-oxadiazol-2-yl)-4-(4-(trifluoromethyl)thiazol-2-yl)-3,3′-bipyridin-6-yl)urea (Example 6, 130 mg, 0.27 mmol) in ethanol (5 mL), N,N-dimethylpropane-1,3-diamine (41.7 mg, 0.41 mmol) was added and microwaved at 100° for 2 h. The reaction was concentrated and the crude resulted was taken in acetonitrile (5 mL), and 2,3,4,6,7,8,9,10-octahydropyrimido[1,2-a]azepine (0.081 mL, 0.54 mmol) (DBU) was added to it. Then triphenyl phosphine (143 mg, 0.54 mmol) followed by perchloromethane (0.053 mL, 0.54 mmol) (carbon tetrachloride) were added to the resulting solution and stirred over the weekend at room temperature. The solvent was removed and the crude was partitioned between water and ethyl acetate. The layers were separated. The aqueous layer was saturated with sodium chloride and extracted with ethyl acetate. The combined layers were washed with brine and dried over magnesium sulfate, concentrated and purified by normal phase (2% MeOH in DCM to 15% MeOH in DCM with 1% ammonium hydroxide). The fractions containing the products were combined and concentrated to give a white solid (34 mg).

MS (ESP): 562 (M+1) for C₂₄H₂₆F₃N₉O₂S

¹H-NMR (DMSO-d₆) δ: 1.11 (t, 3H); 1.52-1.78 (m, 2H); 2.15 (s, 6H); 2.25-2.35 (m, 2H); 3.10-3.32 (m, 4H); 7.56 (brs, 1H); 7.93 (t, 1H); 8.09 (s, 1H); 8.24 (s, 1H); 8.39 (s, 1H); 8.57 (s, 1H); 8.61 (d, 1H); 9.01 (d, 1H); 9.51 (s, 1H)

### Examples 153-157

The following compounds have been synthesized as described for Example 152 from the starting materials indicated in the table below.

| Ex | Compound | Data | SM |
|---|---|---|---|
| 153 | 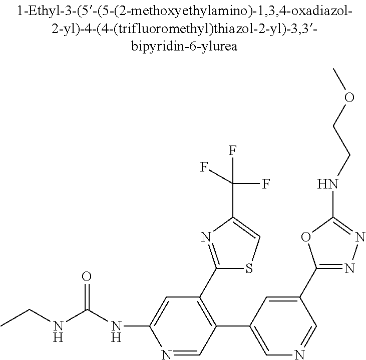 | MS (ESP): 535 (M + 1) for C₂₂H₂₁F₃N₈O₃S ¹H-NMR (DMSO-d₆) δ: 1.11 (t, 3H); 3.12-3.24 (m, 2H); 3.27 (s, 3H); 3.36-3.45 (m, 2H); 3.46-3.52 (m, 2H); 7.57 (brs, 1H); 7.95 (t, 1H); 8.10 (t, 1H); 8.24 (s, 1H); 8.39 (s, 1H); 8.56 (s, 1H); 8.61 (s, 1H); 9.02 (s, 1H); 9.51 (s, 1H) | Example 6 and methoxyethylamine |
| 154 | 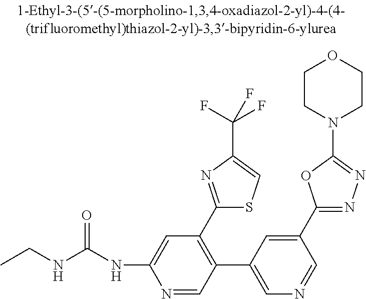 | MS (ESP): 547 (M + 1) for C₂₃H₂₁F₃N₈O₃S ¹H-NMR (DMSO-d₆) δ: 1.11 (t, 3H); 3.10-3.26 (m, 2H); 3.45-3.61 (m, 4H); 3.58-3.84 (m, 4 H); 7.56 (brs, 1H); 8.23 (s, 1H); 8.24 (s, 1H); 8.40 (s, 1H); 8.58 (s, 1H); 8.62 (s, 1H); 9.12 (s, 1H); 9.51 (s, 1H) | Example 6 and morpholine |
| 155 | 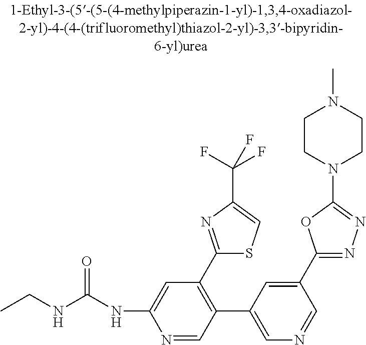 | MS (ESP): 560 (M + 1) for C₂₄H₂₄F₃N₉O₂S ¹H-NMR (DMSO-d₆) δ: 1.11 (t, 3H); 2.32 (s, 3H); 2.30-2.45 (m, 4H); 3.09-3.29 (m, 2H); 3.39-3.73 (m, 4H); 7.57 (brs, 1H); 8.22 (t, 1H); 8.24 (s, 1H); 8.40 (s, 1H); 8.57 (s, 1H); 8.62 (d, 1H); 9.11 (d, 1H); 9.52 (s, 1H) | Example 6 and 1- methylpiperazine |
| 156 | 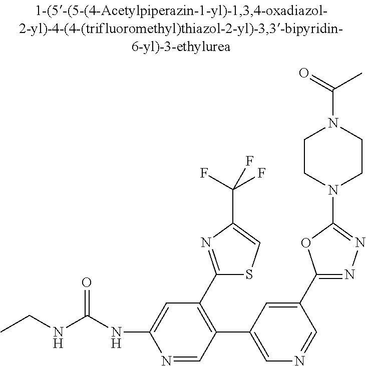 | MS (ESP): 588 (M + 1) for C₂₅H₂₄F₃N₉O₃S ¹H-NMR (DMSO-d₆) δ: 1.11 (t, 3H); 2.05 (s, 3H); 3.08-3.27 (m, 2H); 3.50 (brs, 2H); 3.57 (brs, 6H); 7.56 (brs, 1H); 8.24 (s, 2H); 8.39 (s, 1H); 8.58 (s, 1H); 8.62 (d, 1H); 9.12 (d, 1H); 9.51 (s, 1H) | Example 6 and 1- acetylpiperazine |
| 157 | 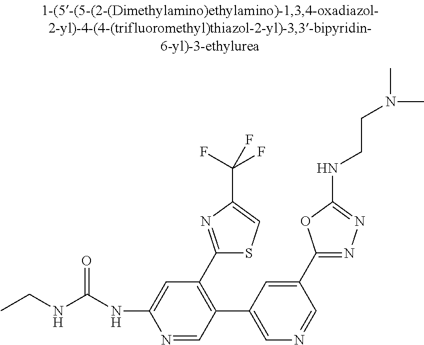 | MS (ESP): 548 (M + 1) for C₂₃H₂₄F₃N₉O₂S ¹H-NMR (DMSO-d₆) δ: 1.11 (t, 3H); 2.17 (s, 6H); 2.35-2.48 (m, 2H); 3.10-3.40 (m, 4H); 7.55 (brs, 1H); 7.82 (t, 1H); 8.09 (d, 1H); 8.25 (s, 1H); 8.39 (s, 1H); 8.56 (s, 1H); 8.61 (d, 1H); 9.02 (d, 1H); 9.48 (s, 1H) | Example 6 and N1,N1- dimethylethane-1,2- diamine |

### Example 158

### 5-(6′-(3-Ethylureido)-4′-(4-(trifluoromethyl)thiazol-2-yl)-3,3′-bipyridin-5-yl)-N,N-dimethyl-2-oxo-1,3,4-oxadiazole-3(2H)-carboxamide

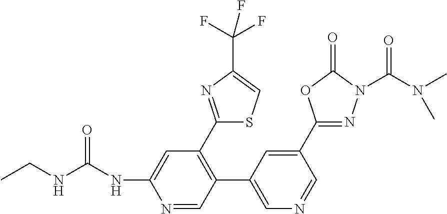

To a solution of 1-ethyl-3-(5′-(5-oxo-4,5-dihydro-1,3,4-oxadiazol-2-yl)-4-(4-(trifluoromethyl)thiazol-2-yl)-3,3′-bipyridin-6-yl)urea (example 6) (150 mg, 0.31 mmol) in THF (3 mL), potassium t-butoxide (0.377 mL, 0.38 mmol) was added at room temperature. It resulted a mixture that was stirred for 15 minutes and then dimethylcarbamic chloride (0.058 mL, 0.63 mmol) was added. Then the resulting mixture was stirred for one hour at room temperature and at 60° C. over night. The solvent was removed and the crude was diluted with water and extracted with ethyl acetate. The organic layer was washed with water several times followed by brine and dried over magesium sulfate, filtered then concentrated and purified by normal phase chromatography to isolate the desired product as a white solid (53 mg).

MS (ESP): 549 (M+1) for C₂₂H₁₉F₃N₈O₄S

¹H-NMR (DMSO-d₆) δ: 1.11 (t, 3H); 3.05 (brs, 6H); 3.15-3.28 (m, 2H); 7.55 (brs, 1H); 8.22 (d, 1H); 8.24 (s, 1H); 8.39 (s, 1H); 8.60 (s, 1H); 8.71 (d, 1H); 9.04 (d, 1H); 9.52 (s, 1H)

### Examples 159-162

The following compounds have been synthesized as described for Example 158 from the starting materials indicated in the table below.

| Ex | Compound | Data | SM |
|---|---|---|---|
| 159 | 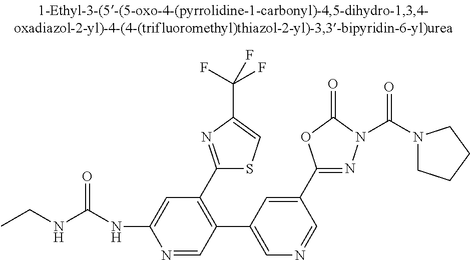 | MS (ESP): 575 (M + 1) for C₂₄H₂₁F₃N₈O₄S ¹H-NMR (DMSO-d₆) δ: 1.11 (t, 3H); 1.87 (br s, 4 H); 3.15-3.28 (m, 2H); 3.46 (brs, 2H); 3.66 (brs, 2H); 7.56 (brs, 1H); 8.23 (s, 2H); 8.39 (s, 1H); 8.60 (s, 1H); 8.71 (s, 1H); 9.04 (s, 1H); 9.52 (s, 1H) | Example 6 and pyrrolidine-1- carbonyl chloride |
| 160 | 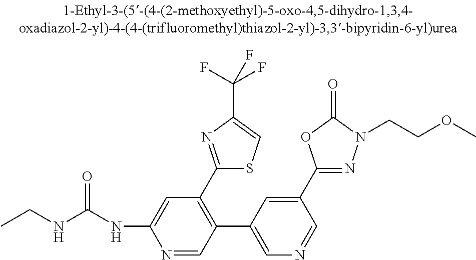 | MS (ESP): 536 (M + 1) for C₂₂H₂₀F₃N₇O₄S ¹H-NMR (DMSO-d₆) δ: 1.11 (t, 3H); 3.15-3.30 (m, 2H); 3.26 (s, 3H); 3.65 (t, 2H); 3.92 (t, 2H); 7.56 (brs, 1H); 8.14 (s, 1H); 8.22 (s, 1H); 8.39 (s, 1H); 8.58 (s, 1H); 8.71 (s, 1H); 9.00 (d, 1H); 9.52 (s, 1H) | Example 6 and 1- bromo-2- methoxyethane |
| 161 | 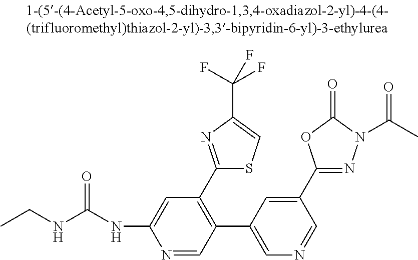 | MS (ESP): 520 (M + 1) for C₂₁H₁₆F₃N₇O₄S ¹H-NMR (DMSO-d₆) δ: 1.11 (t, 3H); 3.15-3.30 (m, 2H); 7.56 (brs, 1H); 8.23 (s, 1H); 8.24 (s, 1H); 8.39 (s, 1H); 8.60 (s, 1H); 8.71 (s, 1H); 9.08 (d, 1H); 9.53 (s, 1H) ¹H-NMR (MeOD₃) δ: 1.11 (t, 3H); 2.59 (s, 3H); 3.32-3.43 (m, 2H); 7.87 (s, 1H); 8.29 (d, 2H); 8.39 (s, 1H); 8.66 (d, 1H); 9.10 (d, 1H) | Example 6 and acetyl chloride |
| 162 | 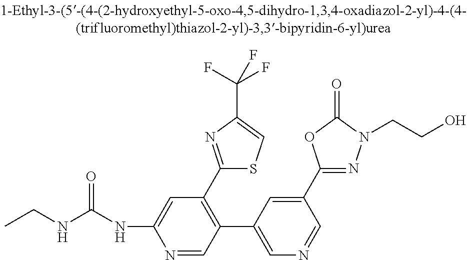 | MS (ESP): 522 (M + 1) for C₂₁H₁₈F₃N₇O₄S ¹H-NMR (DMSO-d₆) δ: 1.11 (t, 3H); 3.11-3.28 (m, 2H); 3.78 (t, 2H); 4.45 (t, 2H); 7.54 (brs, 1H); 8.23 (s, 1H); 8.25 (s, 1H); 8.37 (s, 1H); 8.57 (s, 1H); 8.68 (d, 1H); 9.05 (d, 1H); 9.51 (s, 1H); 11.06 (s, 1H) | Example 6 and (2- bromoethoxy)(tert- butyl)dimethylsilane followed by deprotection with TBAF in THF. |

### Example 163

### 6′-(3-Ethylureido)-N′-hydroxy-4′-(4-(trifluoromethyl)thiazol-2-yl)-3,3′-bipyridine-5-carboximidamide

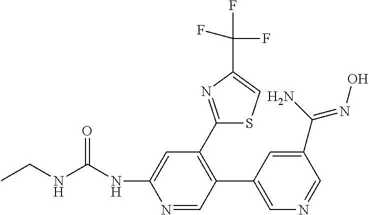

To a suspension of 1-(5′-cyano-4-(4-(trifluoromethyl)thiazol-2-yl)-3,3′-bipyridin-6-yl)-3-ethylurea (Example 2, 70 mg, 0.17 mmol) in ethanol (3 mL), hydroxylamine (0.015 mL, 0.25 mmol) (50% by weight, aq.) was added and microwaved at 80° C. for 1 h. The reaction was concentrated to give a white solid. It was slurried in acetonitrile, filtered and dried to give a white solid (52 mg).

MS (ESP): 452 (M+1) for C₁₈H₁₆F₃N₇O₂S

¹H-NMR (DMSO-d₆) δ: 1.11 (t, 3H); 3.10-3.27 (m, 2H); 6.02 (s, 2H); 7.57 (brs, 1H); 8.03 (s, 1H); 8.26 (s, 1H); 8.35 (s, 1H); 8.46 (d, 1H); 8.56 (s, 1H); 8.93 (d, 1H); 9.48 (s, 1H); 9.91 (s, 1H)

### Example 164

### 6′-(3-Ethylureido)-4′-(4-(trifluoromethyl)thiazol-2-yl)-3,3′-bipyridine-5-carboximidamide

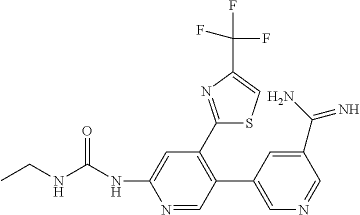

To a solution of sodium methoxide (50 μl, 0.22 mmol) (25% by wt solution in MeOH) in MeOH (3 mL), 1-(5′-cyano-4-(4-(trifluoromethyl)thiazol-2-yl)-3,3′-bipyridin-6-yl)-3-ethylurea (Example 2, 85 mg, 0.20 mmol) was added and the resulting mixture was stirred at room temperature for 4 hours. Then ammonium chloride (13.04 mg, 0.24 mmol) was added and the mixture was stirred overnight at room temperature. As there was no reaction the reaction mixture was transferred to a microwave vial and microwaved at 80° for 20 minutes.

The reaction was complete. The solid formed was filtered off and the washed with acetonitrile and dichloromethane. Then the solid was taken in water and sodium bicarbonate was added to it. The mixture was extracted with ethyl acetate. The extract was washed with water and dried over magnesium sulfate and concentrated to give a white solid which was slurried in acetonitrile, filtered and dried to give a white solid as the product (35 mg).

MS (ESP): 436 (M+1) for C₁₈H₁₆F₃N₇OS

¹H-NMR (DMSO₆) δ: 1.11 (t, 3H); 3.10-3.27 (m, 2H); 6.44 (brs, 2H); 6.55 (brs, 1H); 7.59 (brs, 1H); 8.17 (brs, 1H); 8.26 (s, 1H); 8.35 (s, 1H); 8.52 (s, 1H); 8.56 (s, 1H); 9.06 (brs, 1H); 9.48 (s, 1H)

### Example 165

### S)-1-(5′-(5-(1-Amino-2-methylpropyl)-1,3,4-oxadiazol-2-yl)-4-(3-(trifluoromethyl)-1H-pyrazol-1-yl)-3,3′-bipyridin-6-yl)-3-ethylurea

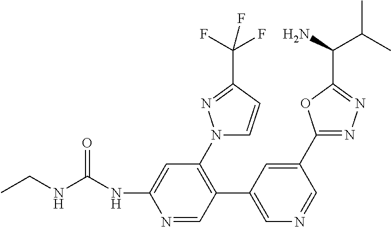

To a solution of (S)-tert-butyl 1-(5-(6′-(3-ethylureido)-4′-(3-(trifluoromethyl)-1H-pyrazol-1-yl)-3,3′-bipyridin-5-yl)-1,3,4-oxadiazol-2-yl)-2-methylpropylcarbamate (Intermediate 257, 65 mg, 0.11 mmol), in dioxane (3 mL), 4M HCl in dioxane (3 mL, 86.39 mmol) was added and the mixture was allowed to stir overnight. The reaction was concentrated and the solid obtained was taken in water, basified with 1N NaOH to precipitate the product.

MS (ESP): 516 (M+1) for C₂₃H₂₄F₃N₉O₂

¹H-NMR (DMSO-d₆) δ: 0.87 (d, 3H); 0.96 (d, 3H); 1.11 (t, 3H); 1.88-208 (m, 1H); 3.10-3.27 (m, 2H); 3.88 (d, 1H); 6.94 (d, 2H); 7.42 (brs, 1H); 7.93 (s, 1H); 7.94 (s, 1H); 7.97 (s, 1H); 8.14 (d, 1H); 8.50 (d, 1H); 8.57 (s, 1H); 9.12 (d, 1H); 9.62 (s, 1H).

### Example 166

### 1-(5′-(5-Cyclopropyl-1,3,4-oxadiazol-2-yl)-4-(4-(trifluoromethyl)thiazol-2-yl)-3,3′-bipyridin-6-yl)-3-ethylurea

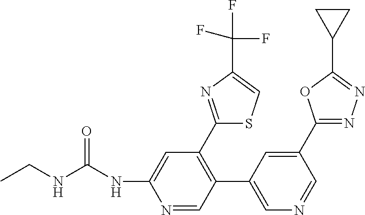

To a suspension of 1-(5′-(2-(cyclopropanecarbonyl)hydrazinecarbonyl)-4-(4-(trifluoromethyl)thiazol-2-yl)-3,3′-bipyridin-6-yl)-3-ethylurea (Intermediate 258, 80 mg, 0.15 mmol), triethylamine (0.021 mL, 0.15 mmol) and triphenylphosphine (81 mg, 0.31 mmol) were added followed by carbon tetrachloride (0.015 mL, 0.15 mmol). The resulting mixture was stirred at 40° C. for 1 h, then concentrated and the crude was partitioned between water and ethyl acetate. The layers separated and the organic layer was washed with water and brine, dried over magnesium sulfate and concentrated. The crude was purified by normal phase chromatography (1% MeOH in DCM to 5%). The fractions containing the product were combined, concentrated and lyophilized to give a white solid (42 mg).

MS (ESP): 502 (M+1) for C₂₂H₁₈F₃N₇O₂S

¹H-NMR (DMSO-d₆) δ: 0.72-155 (m, 4H); 1.10 (t, 3H); 2.19-2.46 (m, 1H); 3.08-3.29 (m, 2H); 7.56 (brs, 1H); 8.23 (s, 1H); 8.28 (t, 1H); 8.40 (s, 1H); 8.57 (s, 1H); 8.68 (s, 1H); 9.15 (s, 1H); 9.52 (s, 1H).

### Examples 167-168

The following Examples were synthesized from the general procedure described below from the starting materials in the Table.

### General Procedures

A suspension of corresponding carboxylic acid (0.3 mmol), hydrazine acetate (0.9 mmol) in phosphorus oxychloride (2.5 mL) was heated at 70° C. for 2 h. The solution was then cooled and concentrated to dryness. A solution of saturated potassium carbonate was added to the crude and extracted with ethyl acetate (3×). The combined organic layers were washed with brine and dried over sodium sulfate. The solvent was removed under vacuum and the crude was purified by Analogix using dichloromethane-methanol.

| Ex | Compound | Data | SM |
|---|---|---|---|
| 167 | 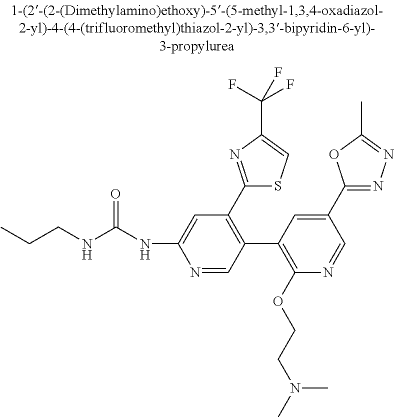 | MS (ESP): 577.2 (MH⁺) for C₂₅H₂₇F₃N₈O₃S ¹H NMR (300 MHz, CD₃OD): δ 0.97-1.02 (m, 3H), 1.58-1.65 (m, 2H), 2.62, (s, 3H), 2.67 (s, 6H), 3.23-3.28 (m, 4H), 4.50 (m, 2H), 7.94 (s, 1H), 8.24 (s, 1H), 8.32-8- 34 (m, 2H), 8.89-8.90 (m, 1H) ¹⁹F NMR (300 MHz, CD₃OD): δ - 65.94 | Intermediate 259 |
| 168 | 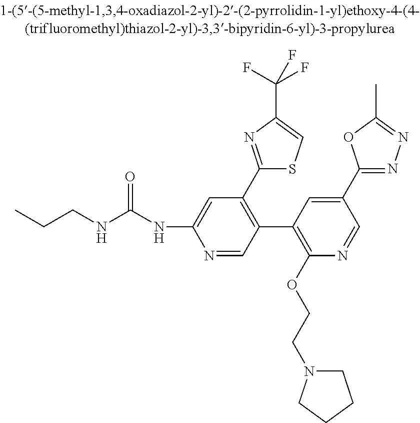 | MS (ESP): 606.3 (MH⁺) for C₂₇H₂₉F₃N₈O₃S ¹H NMR (300 MHz, CD₃OD): δ 0.92-0.95 (m, 3H), 1.09-1.10 (m, 2H), 1.21-1.28 (m, 4H), 1.58-1.65 (m, 2H), 1.99 (m, 2H), 2.11 (m, 4H), 2.62 (s, 3H), 3.63 (m, 2H), 3.93-3.96 (m, 2H), 7.98 (s, 1H), 8.25 (m, 1H), 8.33-8.35 (m, 2H), 8.91 (m, 1H) ¹⁹F NMR (300 MHz, CD₃OD): δ −65.94 | Intermediate 260 |

### Examples 169

### 1-(5′-(5-Methyl-1,3,4-oxadiazol-2-yl)-2′-(tetrahydro-2H-pyran-4-yloxy)-4-(4-(trifluoromethyl)thiazol-2-yl)-3,3′-bipyridin-6-yl)-3-propylurea

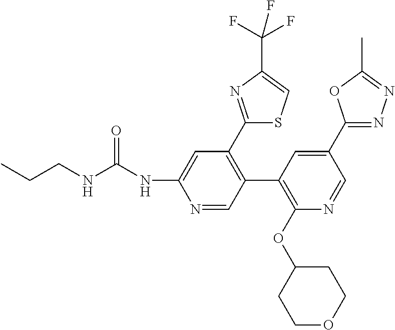

A suspension of 6′-(3-propylureido)-2-(tetrahydro-2H-pyran-4-yloxy)-4′-(4-(trifluoromethyl)thiazol-2-yl)-3,3′-bipyridine-5-carboxylic acid (Intermediate 262, ˜0.3 mmol) and hydrazine acetate (0.9 mmol) in phosphorus oxychloride (2.5 mL) was heated at 70° C. for 2 h. The solution was then cooled and concentrated to dryness. A solution of saturated potassium carbonate was added to the crude and extracted with ethyl acetate (3×). The combined organic layers were washed with brine and dried over sodium sulfate. The solvent was removed under vacuum and the crude was purified by Analogix using dichloromethane-methanol.

MS (ESP): 590.1 (MH⁺) for C₂₆H₂₆F₃N₇O₄S

¹H NMR (300 MHz, CDCl₃): δ 1.002-1.05 (m, 3H), 1.25-1.28 (m, 2H), 1.65-1.75 (m, 4H), 2.62 (s, 3H), 3.39-3.47 (m, 4H), 3.64-3.68 (m, 2H), 5.10-5.18 (m, 1H), 7.61 (s, 1H), 7.73 (s, 1H), 8.20 (s, 1H), 8.24-8.26 (m, 1H), 8.83 (m, 1H), 9.12 (bs, 1H), 9.48 (bs, 1H)

¹⁹F NMR (300 MHz, CDCl₃): δ −64.51

### Examples 170-172

The following Examples were prepared by the general procedure described below from the starting materials indicated in the Table.

### General Procedure

A suspension of corresponding carboxylic acid (0.3 mmol) in thionyl chloride (2 mL) was heated at 50° C. for 1 h. The solution was then cooled and concentrated to dryness. The crude suspended in tetrahydrofuran (2 mL) was added slowly to a solution of hydrazine/tetrahydrofuran (1/2 vol., 3 mL) and stirred at room temperature for 12 h. After this period of time, the crude was concentrated to dryness and purified by reverse phase on Analogix C18-column (water-methanol) to give (˜60%) hydrazides as off-white solids. A suspension of corresponding hydrazide (0.3 mmol) in anhydrous tetrahydrofuran (2 mL) was treated with triethyl amine (0.6 mmol) and 1,1′-carbonyl diimidazole (0.12 mmol). The reaction was stirred at room temperature for 12 h, concentrated to dryness and purified directly by Analogix using dichloromethane-methanol to give (˜50%) product as an off-white solid.

| Ex | Compound | Data | SM |
|---|---|---|---|
| 170 | 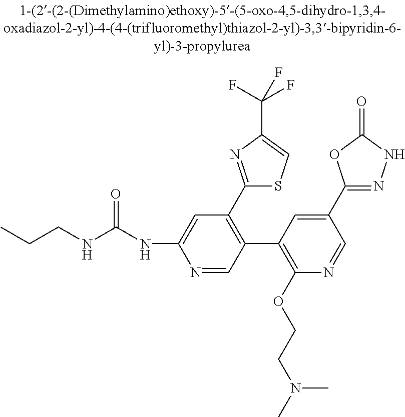 | MS (ESP): 579.3 (MH⁺) for C₂₄H₂₅F₃N₈O₄S ¹H NMR (300 MHz, DMSO-d₆): δ 0.88-0.93 (m, 3H), 1.51-1.53 (m, 2H), 2.20 (s, 6H), 2.50-2.54 (m, 2H), 3.11-3.18 (m, 4H), 4.23 (m, 2H), 7.62 (m, 1H), 8.15 (m, 1H), 8.25-8.29 (m, 2H), 8.55 (m, 1H), 8.66-8.67 9 (m, 1H), 9.47 (s, 1H). ¹⁹F NMR (300 MHz, DMSO-d₆): δ −62.86 | Intermediate 259 |
| 171 | 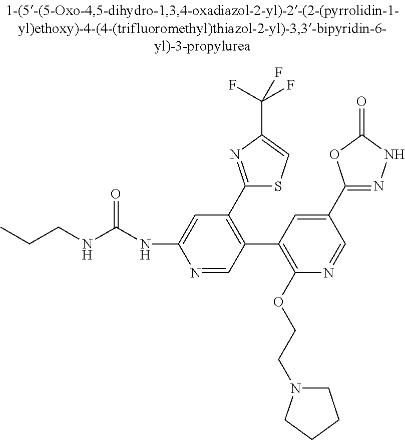 | MS (ESP): 605.1 (MH⁺) for C₂₆H₂₇F₃N₈O₄S ¹H NMR (300 MHz, DMSO-d₆): δ 0.88-0.93 (m, 3H), 1.46-1.48 (m, 2H), 1.53-1.62 (m, 4H), 2.49 (m, 4H), 2.51 (m, 2H), 3.11-3.18 (m, 2H), 4.22 (m, 2H), 7.62-7.65 (m, 1H), 8.16 (s, 1H), 8.26-8.29 (m, 2H), 8.52-8.55 (m, 1H), 8.66-8.67 (m, 1H), 9.47 (s, 1H). ¹⁹F NMR (300 MHz, dmso-d₆): δ - 62.85 | Intermediate 260 |
| 172 | 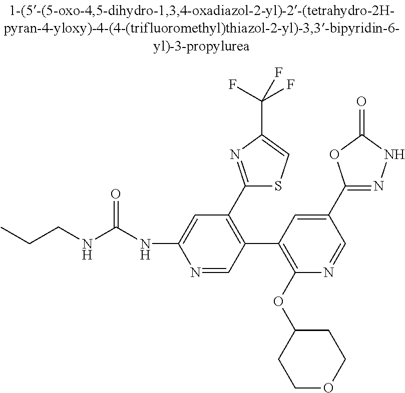 | MS (ESP): 592.2 (MH⁺) for C₂₅H₂₄F₃N₇O₅S ¹H NMR (300 MHz, CD₃OD): δ 0.97-1.02 (m, 3H), 1.28 (m, 2H), 1.59-1.64 (m, 2H), 1.66-1.70 (m, 2H), 3.26-3.30 (m, 2H), 3.38-3.41 (m, 2H), 3.44-3.59 (m, 2H), 5.14- 5.16 (m, 1H), 7.87 (s, 1H), 8.14- 8.15 (m, 1H), 8.20 (s, 1H), 8.24 (s, 1H), 8.56-8.65 (m, 1H) ¹⁹F NMR (300 MHz, CD₃OD): δ - 64.82 | Intermediate 261 |

### Example 173

### 1-(5′-(5-methyl-1,3,4-oxadiazol-2-yl)-4-(4-(trifluoromethyl)thiazol-2-yl)-3,3′-bipyridin-6-yl)-3-propylurea

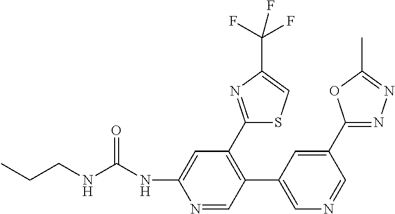

Methyl 6′-(3-propylureido)-4′-(4-(trifluoromethyl)thiazol-2-yl)-3,3′-bipyridine-5-carboxylate (Intermediate 263, 140 mg) was dissolved in tetrahydrofuran (3 mL) and methanol (3 mL).

1N Sodium hydroxide (3 mL) was added, and the reaction was stirred at room temperature for 3 h. The organics were removed and the residual aqueous phase was acidified to pH ˜2 with 1N hydrochloric acid. The mixture was filtered and the solid dried in a vacuum oven at 50° C. for 18 h. The solid was then dissolved in phosphorous oxychloride (3 mL), acetic hydrazide (25 mg) was added and the solution heated at 60° C. for 3 h. Most of the phosphorous oxychloride was removed in vacuo and then saturated sodium bicarbonate was added to the mixture to obtain a pH ˜7. The solution was extracted with 2:1 ethyl acetate: tetrahydrofuran (3×, 3 mL each). The organic phases were combined, dried over sodium sulfate, and the solvent was removed in vacuo.

MS (ESP): 490.2 (M+H⁺) for C₂₁H₁₈F₃N₇O₂S

¹H NMR (300 MHz, DMSO-d₆): δ 0.91 (t, 3H), 1.46-1.54 (m, 2H), 2.60 (s, 3H), 3.12-3.18 (m, 2H), 7.64 (bt, 1H), 8.24 (s, 1H), 8.30 (dd, 1H), 8.41 (d, 1H), 8.58 (d, 1H), 8.70 (d, 1H), 9.17 (d, 1H), 9.54 (bs, 1H)

### Example 174

### 1-(5-(4,7-dihydroxythiazolo[5,4-d]pyridazin-2-yl)-4-(4-(trifluoromethyl)thiazol-2-yl)pyridin-2-yl)-3-propylurea

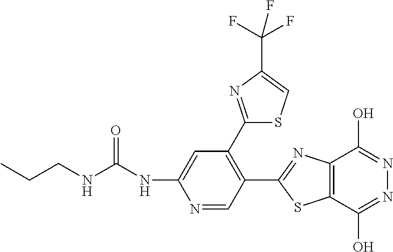

Diethyl 2-(6-(3-propylureido)-4-(4-(trifluoromethyl)thiazol-2-yl)pyridin-3-yl)thiazole-4,5-dicarboxylate (Intermediate 264, 73 mg, 0.13 mmol), was dissolved in methanol (10 mL) and hydrazine (0.4 mL) was added. The reaction was heated at reflux for 3 h. 12 M Hydrochloric acid (1 mL) was then added and the reaction heated for a further 2 h. The solvents were removed in vacuo. The residue was chromatographed on the preparative HPLC to give 18 mg (26% yield) of 1-(5-(4,7-dihydroxythiazolo[5,4-d]pyridazin-2-yl)-4-(4-(trifluoromethyl)thiazol-2-yl)pyridin-2-yl)-3-propylurea as a tan solid.

MS (ESP): 498.0 (M+H⁺) for C₁₈H₁₄F₃N₇O₃S₂

¹H NMR (300 MHz, DMSO-d₆): δ 0.93 (t, 3H), 1.46-1.53 (m, 2H), 2.60 (s, 3H), 3.11-3.15 (m, 2H), 7.52 (bt, 1H), 8.17 (s, 1H), 8.71 (d, 1H), 8.78 (s, 1H), 9.76 (bs, 1H).

### Example 175

### 1-(2′-(5-hydroxy-1,3,4-oxadiazol-2-yl)-4-(4-(trifluoromethyl)thiazol-2-yl)-3,4′-bipyridin-6-yl)-3-propylurea

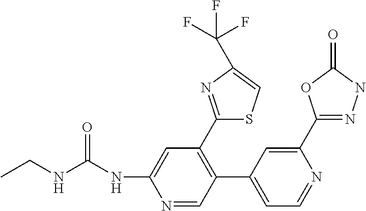

Methyl 6-(3-propylureido)-4-(4-(trifluoromethyl)thiazol-2-yl)-3,4′-bipyridine-2′-carboxylate (Intermediate 265, 65 mg, 0.14 mmol) was dissolved in ethanol (10 mL) and hydrazine monohydrate (1 mL) was added. The reaction was heated at reflux for 6 h. The solvent was removed in vacuo, and the residue was placed in a vacuum oven at 60° C. for 1 h. The residue was then dissolved in anhydrous tetrahydrofuran (10 mL). 1,1′-Carbonyl diimidazole (100 mg) was added and the reaction was stirred at 25° C. for 18 h. The solvent was removed in vacuo and the residue was subjected to preparative HPLC. This gave 19 mg (27% yield) of 1-(2′-(5-hydroxy-1,3,4-oxadiazol-2-yl)-4-(4-(trifluoromethyl)thiazol-2-yl)-3,4′-bipyridin-6-yl)-3-propylurea as a white powder.

MS (ESP): 492.0 (M+H⁺) for C₂₀H₁₇F₃N₆O₃S

¹H NMR (300 MHz, CD₃OD): δ 0.99 (t, 3H), 1.61-1.63 (m, 2H), 3.11-3.15 (m, 2H), 7.44 (dd, 1H), 7.80 (s, 1H), 7.84 (dd, 1H), 8.28 (d, 1H), 8.39 (d, 1H) 8.62 (dd, 1H)

### Examples 176-177

The following Examples were synthesized according to the procedure for Example 158 from the starting materials indicated in the Table.

| Ex | Compound | Data | SM |
|---|---|---|---|
| 176 | 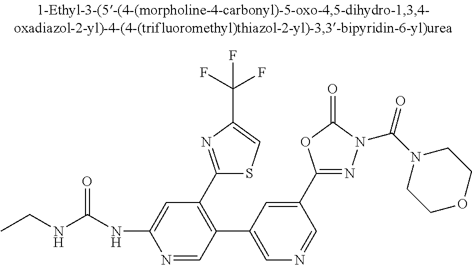 | MS (ESP): 590.99 (M⁺) for C₂₄H₂₁F₃N₈O₅S ¹H-NMR (300 MHz, DMSO-d₆) δ 1.11 (t, J = 7.20 Hz, 3H); 3.14- 3.28 (m, 2H); 3.51-3.63 (m, 4H); 3.62-3.72 (m, 4H); 7.54 (br. s., 1H); 8.21 (t, J = 2.15 Hz, 1H); 8.23 (s, 1H); 8.38 (s, 1H); 8.60 (s, 1H); 8.71 (d, J = 2.27 Hz, 1H); 9.04 (d, J = 2.02 Hz, 1H); 9.50 (s, 1H). | Example 6 and morpholine-4- carbonyl chloride |
| 177 | 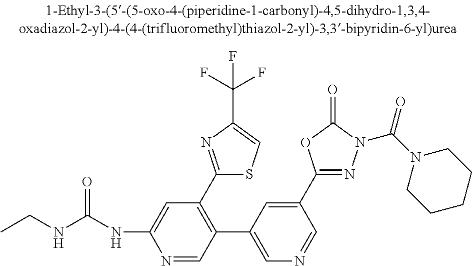 | MS (ESP): 588.98 (M⁺) for C₂₅H₂₃F₃N₈O₄S ¹H-NMR (400 MHz, DMSO-d₆) δ: 1.10 (t, J = 7.07 Hz, 3H); 1.50- 1.70 (m, 6H); 3.14-3.27 (m, 2H); 3.48-3.59 (m, 4H); 7.46-7.63 (m, 1H); 8.12-8.28 (m, 2H); 8.38 (s, 1H); 8.60 (s, 1H); 8.70 (d, J = 2.02 Hz, 1H); 9.02 (d, J = 2.02 Hz, 1H); 9.51 (s, 1H) | Example 6 and piperidine-1-carbonyl chloride |

### Examples 178-182

The following Examples were synthesized according to the procedure for Example 166 from the starting materials indicated in the Table.

| Ex | Compound | Data | SM |
|---|---|---|---|
| 178 | 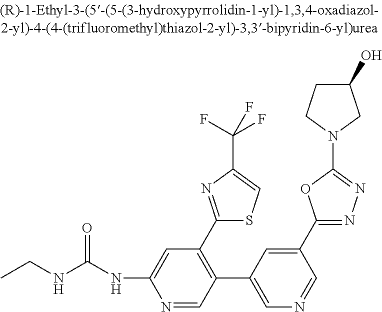 | MS (ESP): 547.11 (M + 1) for C₂₃H₂₁F₃N₈O₃S ¹H-NMR (400 MHz, DMSO-d₆) δ: 1.11 (t, J = 7.07 Hz, 3H); 1.77- 2.14 (m, 2H); 3.11-3.28 (m, 2H); 3.35-3.43 (m, 1H); 3.51-3.66 (m, 3H); 4.41 (br. s., 1H); 5.10 (d, J = 3.79 Hz, 1H); 7.47-7.63 (m, 1H); 8.15 (br. s., 1H); 8.23 (s, 1H); 8.40 (s, 1H); 8.56 (s, 1H); 8.61 (d, J = 2.02 Hz, 1H); 9.07 (d, J = 2.02 Hz, 1H); 9.50 (s, 1H). | Intermediate 267 |
| 179 | 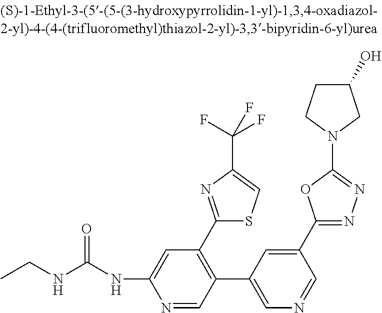 | MS (ESP): 546.99 (M⁺) for C₂₃H₂₁F₃N₈O₃S ¹H-NMR (400 MHz, DMSO-d₆) δ: 1.11 (t, J = 7.20 Hz, 3H); 1.82- 2.12 (m, 2H); 3.13-3.27 (m, 2 H); 3.39 (d, J = 10.86 Hz, 1 H); 3.58 (dd, J = 9.85, 4.55 Hz, 3H); 4.41 (br. s., 1H); 5.11 (d, J = 3.54 Hz, 1H); 7.57 (br. s., 1H); 8.09-8.18 (m, 1H); 8.23 (s, 1H); 8.40 (s, 1H); 8.56 (s, 1H); 8.61 (d, J = 2.02 Hz, 1H); 9.07 (d, J = 2.02 Hz, 1H); 9.50 (s, 1H) | Intermediate 268 |
| 180 | 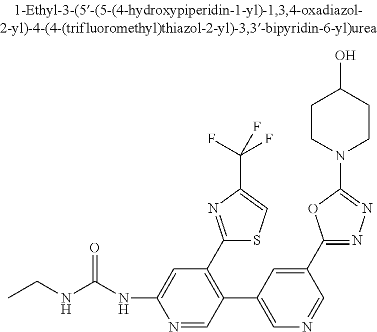 | MS (ESP): 561.16 (M + 1) for C₂₄H₂₃F₃N₈O₃S ¹H-NMR (400 MHz, DMSO-d₆) δ: 1.11 (t, J = 7.20 Hz, 3H); 1.34- 1.57 (m, 2H); 1.70-1.93 (m, 2H); 3.07-3.40 (m, 4H); 3.59-3.97 (m, 3H); 4.82 (d, J = 4.04 Hz, 1H); 7.56 (br. s., 1H); 8.18-8.22 (m, 1H); 8.23 (s, 1H); 8.40 (s, 1H); 8.56 (s, 1H); 8.61 (d, 1H); 9.10 (d, J = 2.02 Hz, 1H); 9.49 (s, 1H) | Intermediate 269 |
| 181 | 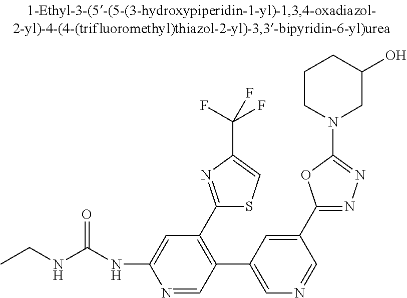 | MS (ESP): 561.16 (M + 1) for C₂₄H₂₃F₃N₈O₃S ¹H-NMR (400 MHz, DMSO-d₆) δ: 1.11 (t, J = 7.20 Hz, 3H); 1.35- 1.62 (m, 2H); 1.69-1.94 (m, 2H); 3.00-3.30 (m, 4H); 3.52-3.72 (m, 2H); 3.70-3.86 (m, 1H); 4.95 (d, J = 4.04 Hz, 1H); 7.56 (br. s., 1H); 8.21 (t, J = 2.02 Hz, 1H); 8.24 (s, 1H); 8.39 (s, 1H); 8.56 (s, 1H); 8.60 (d, J = 2.02 Hz, 1H); 9.09 (d, J = 2.02 Hz, 1H); 9.49 (s, 1H) | Intermediate 270 |
| 182 | 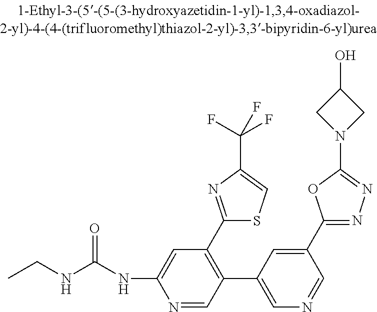 | MS (ESP): 533.01 (M + 1) for C₂₂H₁₉F₃N₈O₃S ¹H-NMR (400 MHz, DMSO-d₆) δ: 1.11 (t, J = 7.07 Hz, 3H); 3.13- 3.27 (m, J = 7.07, 6.76, 6.60, 6.60 Hz, 2H); 3.94 (dd, J = 8.59, 4.80 Hz, 2H); 4.35 (t, J = 7.58 Hz, 2H); 4.54-4.73 (m, 1H); 5.87 (d, J = 6.57 Hz, 1H); 7.45-7.64 (m, 1H); 8.13 (t, J = 2.15 Hz, 1H); 8.23 (s, 1H); 8.39 (s, 1H); 8.57 (s, 1H); 8.62 (d, J = 2.02 Hz, 1H); 9.05 (d, J = 2.02 Hz, 1H); 9.49 (s, 1H) | Intermediate 266 |

### Examples 183-184

The following Examples were synthesized according to the procedure for Example 165 from the starting materials indicated in the Table.

| Ex | Compound | Data | SM |
|---|---|---|---|
| 183 | 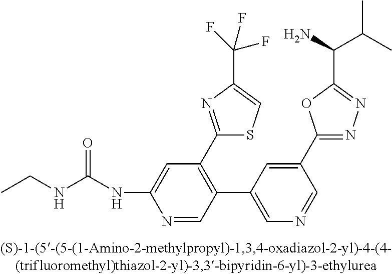 | MS (ESP): 533.02 (M + 1) for C₂₃H₂₃F₃N₈O₂S ¹H-NMR (400 MHz, DMSO-d₆) δ: 0.87 (d, J = 6.82 Hz, 3H); 0.95 (d, J = 6.82 Hz, 3H); 1.11 (t, J = 7.20 Hz, 3H); 1.90-2.19 (m, 3H); 3.14-3.27 (m, 2H); 3.89 (br. s., 1H); 7.55 (br. s., 1H); 8.23 (s, 1H); 8.30 (d, J = 1.01 Hz, 1H); 8.42 (s, 1H); 8.57 (s, 1H); 8.72 (d, J = 1.26 Hz, 1H); 9.20 (d, J = 1.26 Hz, 1H); 9.51 (s, 1H) | Intermediate 271 followed by HCl |
| 184 | 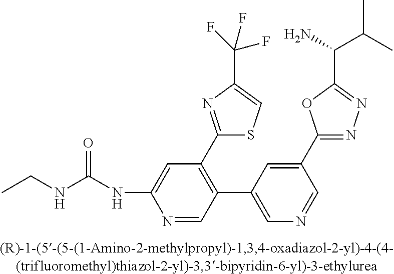 | MS (ESP): 533.01 (M + 1) for C₂₃H₂₃F₃N₈O₂S ¹H-NMR (400 MHz, DMSO-d₆) δ: 0.87 (d, J = 6.57 Hz, 2H); 0.95 (d, J = 6.82 Hz, 2H); 1.11 (t, J = 7.07 Hz, 3H); 1.95-2.08 (m, 1H); 2.06-2.21 (m, 1H); 3.21 (dq, J = 6.95, 6.69 Hz, 1H); 3.88 (d, J = 6.32 Hz, 1H); 7.55 (br. s., 1H); 8.23 (s, 1H); 8.30 (t, J = 1.77 Hz, 1H); 8.42 (s, 1H); 8.57 (s, 1H), 8.72 (d, J = 1.77 Hz, 1H); 9.20 (d, J = 1.26 Hz, 1H); 9.51 (s, 1H) | Intermediate 272 followed by HCl |

### Example 185

The following Example was prepared according to the procedure described for Intermediate 2 using the starting materials indicated in the table.

| Ex | Compound | Data | SM |
|---|---|---|---|
| 185 | 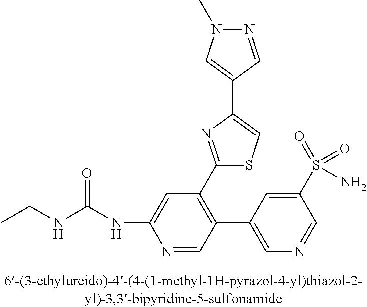 | LC/MS (ES⁺)[(M + H)⁺]: 485 for C₂₀H₂₀N₈O₃S₂. ¹H NMR (300 MHz, d₆-DMSO): 1.11 (t, 3H), 3.21 (m, 2H), 3.84 (s, 3H), 6.96 (s, 2H), 7.64 (t, 1H), 7.68 (s, 1H), 7.78 (s, 1H), 7.92 (s, 1H), 8.13 (t, 1H), 8.21 (s, 1H), 8.27 (s, 1H), 8.60 (d, 1H), 8.94 (d, 1H), 9.49 (s, 1H). | Intermediate 302 and 5-bromopyridine-3- sulfonamide |

### Examples 186-227

The following Examples were prepared according to the procedure described for Example 33 using the starting materials indicated in the table.

| Ex | Compound | Data | SM |
|---|---|---|---|
| 186 | 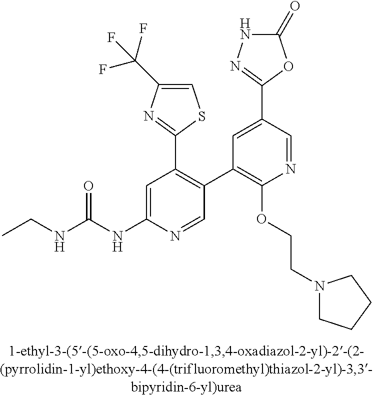 | LC/MS (ES⁺)[(M + H)⁺]: 591 for C₂₅H₂₅F₃N₈O₄S. ¹H NMR (300 MHz, d₆-DMSO): 1.04 (t, 3H), 1.48 (m, 4H), 2.21 (m, 4H), 2.30 (t, 1H), 3.14 (m, 2H), 4.07 (t, 2H), 7.50 (t, 1H), 8.80 (d, 1H), 8.17 (s, 1H), 8.21 (s, 1H), 8.48 (s, 1H), 8.58 (d, 1H), 9.40 (s, 1H), 12.30 (s, 1H). | Intermediate 387 |
| 187 | 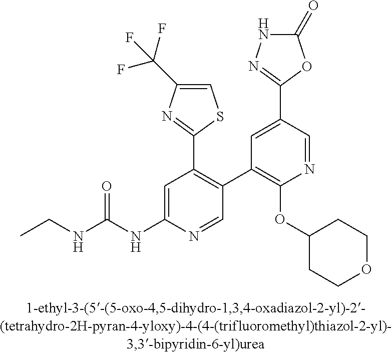 | LC/MS (ES⁺)[(M + H)⁺]: 578 for C₂₄H₂₂F₃N₇O₅S. ¹H NMR (300 MHz, d₆-DMSO): 1.09 (m, 2H), 1.11 (t, 3H), 1.62 (m, 2H), 3.21 (m, 2H), 3.34 (m, 2H), 3.45 (m, 2H), 5.06 (m, 1H), 7.60 (t, 1H), 8.18 (m, 1H), 8.21 (s, 1H), 8.28 (s, 1H), 8.55 (s, 1H), 8.63 (s, 1H), 9.49 (s, 1H), 12.67 (s, 1H). | Intermediate 287 |
| 188 | 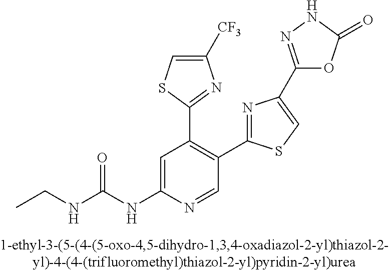 | LC/MS (ES⁺)[(M + H)⁺]: 484 for C₁₇H₁₂F₃N₇O₃S₂. ¹H NMR (300 MHz, d₆-DMSO): 1.11 (t, 3H), 3.20 (m, 2H), 7.48 (t, 1H), 8.15 (s, 1H), 8.48 (s, 1H), 8.69 (s, 1H), 9.68 (s, 1H), 12.67 (s, 1H). | Intermediate 351 |
| 189 | 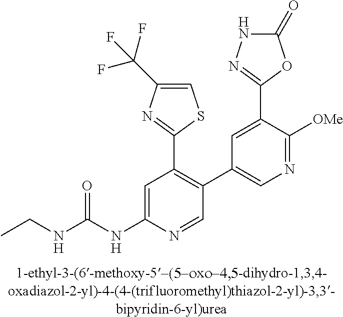 | LC/MS (ES⁺)[(M + H)⁺]: 508 for C₂₀H₁₆F₃N₇O₄S. ¹H NMR (300 MHz, d₆-DMSO): 1.11 (t, 3H), 3.21 (m, 2H), 4.02 (s, 3H), 7.61 (m, 1H), 8.03 (s, 1H), 8.25 (s, 1H), 8.33 (s, 2H), 8.58 (s, 1H), 9.46 (s, 1H), 12.67 (s, 1H). | Intermediate 352 |
| 190 | 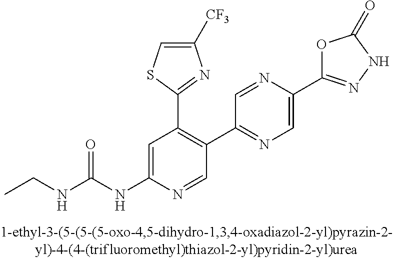 | LC/MS (ES⁺)[(M + H)⁺]: 479 for C₁₈H₁₃F₃N₈O₃S. ¹H NMR (300 MHz, d₆-DMSO): 1.04 (t, 3H), 3.15 (m, 2H), 6.96 (s, 1H), 7.47 (m, 1H), 8.09 (s, 1H), 8.54 (s, 1H), 8.55 (s, 1H), 8.81 (s, 1H), 8.98 (s, 1H), 9.57 (s, 1H). | Intermediate 353 |
| 191 | 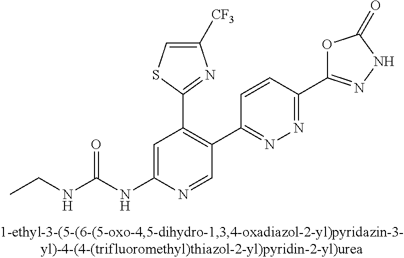 | LC/MS (ES⁺)[(M + H)⁺]: 479 for C₁₈H₁₃F₃N₈O₃S. ¹H NMR (300 MHz, d₆-DMSO): 1.12 (t, 3H), 3.23 (m, 2H), 7.54 (m, 1H), 7.97 (d, 1H), 8.19 (m, 2H), 8.58 (s, 1H), 8.63 (s, 1H), 8.83 (s, 1H), 9.63 (s, 1H). | Intermediate 388 |
| 192 | 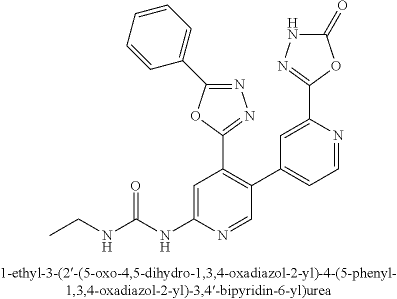 | LC/MS (ES⁺)[(M + H)⁺]: 471 for C₂₃H₁₈N₈O₄. ¹H NMR (300 MHz, d₆-DMSO): 1.05 (t, 3H), 3.16 (m, 2H), 7.21 (dd, 1H), 7.46 (m, 3H), 7.52 (m, 1H), 7.62 (m, 2H), 7.71 (s, 1H), 8.34 (d, 2H), 8.47 (d, 1H), 9.53 (s, 1H). | Intermediate 354 |
| 193 | 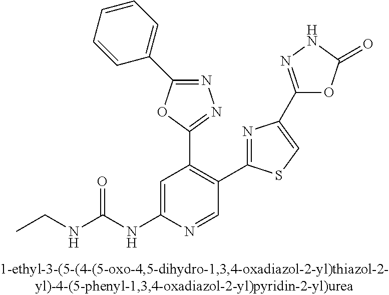 | LC/MS (ES⁺)[(M + H)⁺]: 476 for C₂₁H₁₆N₈O₄S. ¹H NMR (300 MHz, d₆-DMSO): 1.05 (t, 3H), 3.15 (m, 2H), 7.39 (t, 1H), 7.49 (m, 3H), 7.74 (m, 2H), 8.27 (s, 1H), 8.42 (s, 1H), 8.70 (s, 1H), 9.72 (s, 1H), 12.50 (s, 1H). | Intermediate 355 |
| 194 | 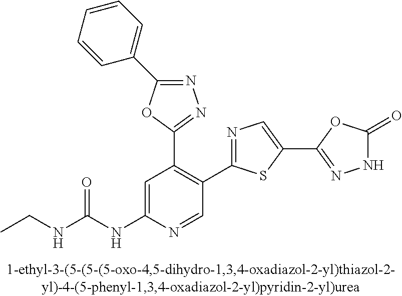 | LC/MS (ES⁺)[(M + H)⁺]: 477 for C₂₁H₁₆N₈O₄S. ¹H NMR (300 MHz, d₆-DMSO): 1.04 (t, 3H), 3.15 (m, 2H), 7.39 (t, 1H), 7.55 (m, 3H), 7.78 (m, 2H), 8.23 (s, 1H), 8.26 (s, 1H), 8.74 (s, 1H), 9.74 (s, 1H), 12.74 (s, 1H). | Intermediate 356 |
| 195 | 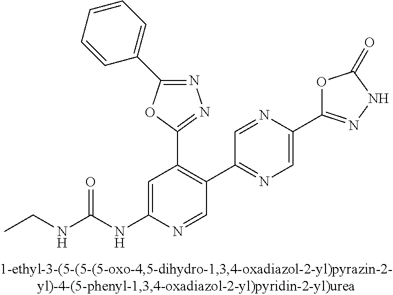 | LC/MS (ES⁺)[(M + H)⁺]: 472 for C₂₂H₁₇N₉O₄. ¹H NMR (300 MHz, d₆-DMSO): 1.12 (t, 3H), 3.23 (m, 2H), 7.48 (t, 1H), 7.60 (m, 3H), 7.79 (m, 2H), 8.39 (s, 1H), 8.73 (s, 1H), 9.06 (d, 1H), 9.17 (d, 1H), 9.73 (s, 1H), 12.82 (s, 1H). | Intermediate 357 |
| 196 | 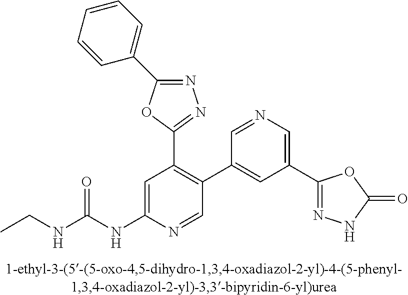 | LC/MS (ES⁺)[(M + H)⁺]: 471 for C₂₃H₁₈N₈O₄. ¹H NMR (300 MHz, d₆-DMSO): 1.11 (t, 3H), 3.12 (m, 2H), 7.01 (s, 1H), 7.51 (t, 1H), 7.56 (m, 3H), 7.74 (d, 2H), 8.28 (d, 1H), 8.41 (s, 1H), 8.47 (s, 1H), 8.79 (d, 1H), 9.02 (d, 1H), 9.57 (s, 1H). | Intermediate 358 |
| 197 | 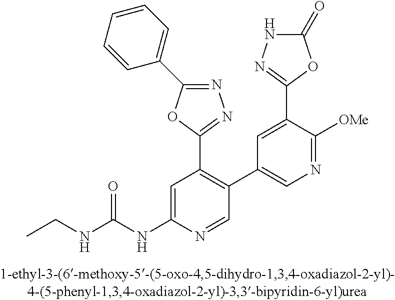 | LC/MS (ES⁺)[(M + H)⁺]: 501 for C₂₄H₂₀N₈O₅. ¹H NMR (300 MHz, d₆-DMSO): 1.12 (t, 3H), 3.25 (m, 2H), 4.05 (s, 3H), 7.52 (t, 1H), 7.60 (m, 3H), 7.78 (m, 2H), 8.21 (d, 1H), 8.40 (s, 1H), 8.44 (d, 1H), 8.45 (s, 1H), 9.54 (s, 1H), 12.67 (s, 1H). | Intermediate 359 |
| 198 | 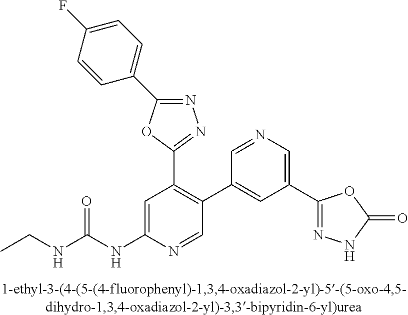 | LC/MS (ES⁺)[(M + H)⁺]: 489 for C₂₃H₁₇FN₈O₄. ¹H NMR (300 MHz, d₆-DMSO): 1.12 (t, 3H), 3.23 (m, 2H), 7.45 (m, 3H), 7.82 (m, 2H), 8.28 (s, 1H), 8.42 (s, 1H), 8.47 (s, 1H), 8.81 (s, 1H), 9.02 (s, 1H), 9.57 (s, 1H), 12.82 (s, 1H). | Intermediate 360 |
| 199 | 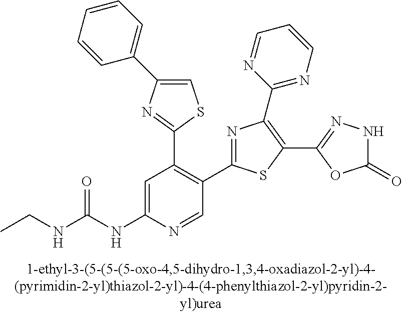 | LC/MS (ES⁺)[(M + H)⁺]: 570 for C₂₆H₁₉N₉O₃S₂. ¹H NMR (300 MHz, d₆-DMSO): 1.05 (t, 3H), 3.15 (m, 2H), 7.33 (m, 2H), 7.50 (m, 1H), 7.51 (m, 1H), 7.77 (d, 2H), 8.16 (s, 1H), 8.31 (s, 1H), 8.68 (s, 1H), 8.86 (d, 2H), 9.62 (s, 1H), 12.71 (s, 1H). | Intermediate 361 |
| 200 | 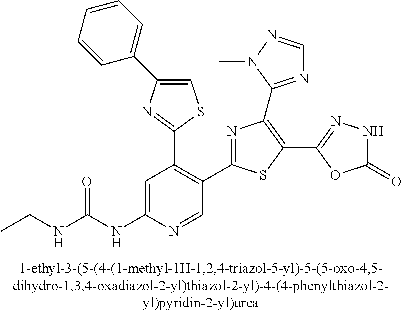 | LC/MS (ES⁺)[(M + H)⁺]: 573 for C₂₅H₂₀N₁₀O₃S₂. ¹H NMR (300 MHz, d₆-DMSO): 1.04 (t, 3H), 3.13 (m, 2H), 3.69 (s, 3H), 7.32 (m, 3H), 7.47 (m, 1H), 7.76 (d, 2H), 8.01 (s, 1H), 8.10 (s, 1H), 8.32 (s, 1H), 8.73 (s, 1H), 9.65 (s, 1H), 12.80 (s, 1H). | Intermediate 362 |
| 201 | 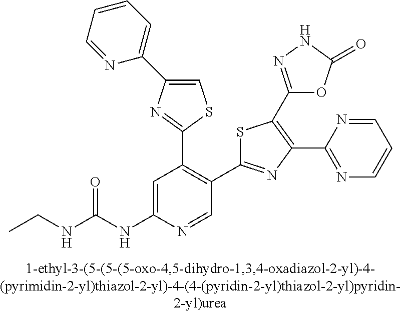 | LC/MS (ES⁺)[(M + H)⁺]: 571 for C₂₅H₁₈N₁₀O₃S₂. ¹H NMR (300 MHz, d₆-DMSO): 1.12 (t, 3H), 3.21 (m, 2H), 7.37 (m, 1H), 7.57 (m, 2H), 7.83 (m, 2H), 8.24 (m, 1H), 8.50 (m, 1H), 8.63 (m, 1H), 8.77 (m, 1H), 8.93 (m, 2H), 9.70 (s, 1H), 12.77 (s, 1H). | Intermediate 363 |
| 202 | 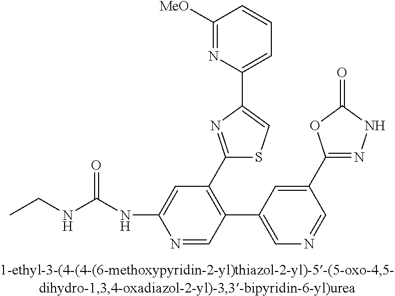 | LC/MS (ES⁺)[(M + H)⁺]: 517 for C₂₄H₂₀N₈O₄S. ¹H NMR (300 MHz, d₆-DMSO): 1.11 (t, 3H), 3.22 (m, 2H), 3.91 (s, 3H), 6.78 (d, 1H), 7.25 (d, 1H), 7.61 (m, 1H), 7.72 (t, 1H), 8.20 (m, 1H), 8.27 (s, 1H), 8.34 (d, 2H), 8.69 (d, 1H), 8.99 (d, 1H), 9.50 (s, 1H), 12.80 (s, 1H). | Intermediate 364 |
| 203 | 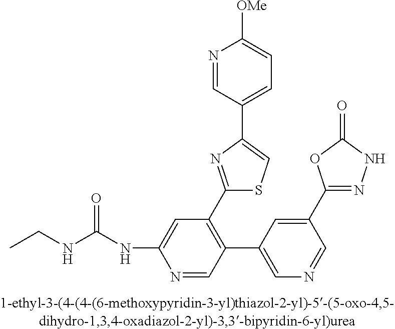 | LC/MS (ES⁺)[(M + H)⁺]: 517 for C₂₄H₂₀N₈O₄S. ¹H NMR (300 MHz, d₆-DMSO): 1.11 (t, 3H), 3.22 (m, 2H), 3.88 (s, 3H), 6.84 (d, 1H), 7.62 (m, 1H), 8.01 (m, 1H), 8.20 (m, 1H), 8.21 (s, 1H), 8.26 (s, 1H), 8.34 (s, 1H), 8.52 (d, 1H), 8.69 (d, 1H), 8.99 (d, 1H), 9.49 (s, 1H), 12.80 (s, 1H). | Intermediate 369 |
| 204 | 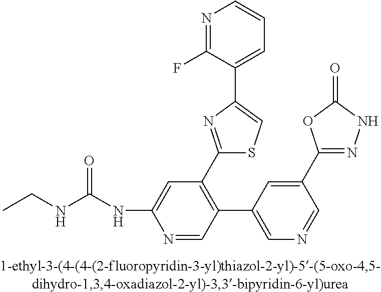 | LC/MS (ES⁺)[(M + H)⁺]: 505 for C₂₃H₁₇FN₈O₃S. ¹H NMR (300 MHz, d₆-DMSO): 1.11 (t, 3H), 3.21 (m, 2H), 7.43 (m, 1H), 7.60 (m, 1H), 8.15 (m, 1H), 8.19 (m, 1H), 8.20 (s, 1H), 8.22 (m, 1H), 8.27 (s, 1H), 8.36 (s, 1H), 8.70 (d, 1H), 8.99 (d, 1H), 9.50 (s, 1H), 12.80 (s, 1H). | Intermediate 389 |
| 205 | 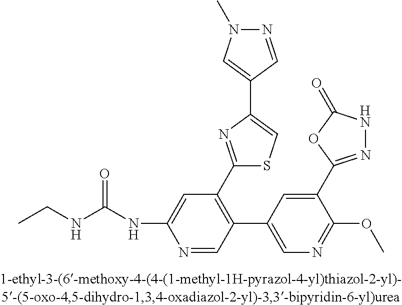 | LC/MS (ES⁺)[(M + H)⁺]: 520 for C₂₃H₂₁N₉O₄S. ¹H NMR (300 MHz, d₆-DMSO): 1.11 (t, 3H), 3.21 (m, 2H), 3.85 (s, 3H), 4.02 (s, 3H), 7.68 (m, 1H), 7.70 (s, 1H), 7.77 (s, 1H), 7.99 (s, 1H), 8.06 (d, 1H), 8.20 (s, 1H), 8.27 (s, 1H), 8.31 (d, 1H), 9.42 (s, 1H), 12.52 (s, 1H). | Intermediate 368 |
| 206 | 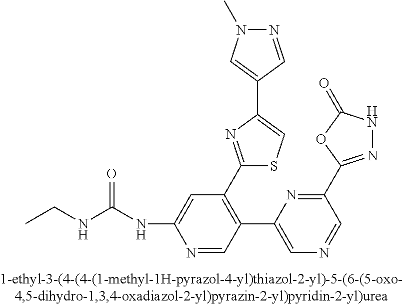 | LC/MS (ES⁺)[(M + H)⁺]: 491 for C₂₁H₁₈N₁₀O₃S. ¹H NMR (300 MHz, d₆-DMSO): 1.11 (t, 3H), 3.21 (m, 2H), 3.80 (s, 3H), 7.54 (s, 1H), 7.57 (m, 1H), 7.79 (s, 1H), 7.82 (s, 1H), 8.15 (s, 1H), 8.52 (s, 1H), 8.79 (d, 1H), 9.10 (d, 1H), 9.57 (s, 1H), 12.98 (s, 1H). | Intermediate 365 |
| 207 | 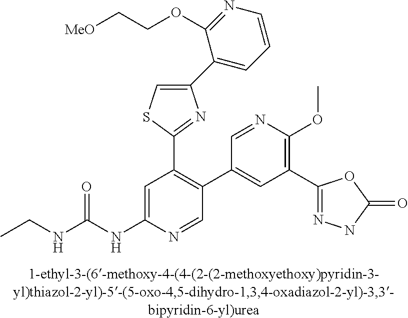 | LC/MS (ES⁺)[(M + H)⁺]: 591 for C₂₇H₂₆N₈O₆S. ¹H NMR (300 MHz, d₆-DMSO): 1.11 (t, 3H), 3.22 (m, 2H), 3.31 (s, 3H), 3.76 (m, 2H), 4.01 (s, 3H), 4.53 (m, 2H), 7.06 (m, 1H), 7.70 (m, 1H), 8.09 (d, 1H), 8.15 (m, 1H), 8.20 (m, 1H), 8.26 (s, 1H), 8.28 (m, 1H), 8.29 (s, 1H), 8.33 (d, 1H), 9.44 (s, 1H), 12.66 (s, 1H). | Intermediate 370 |
| 208 | 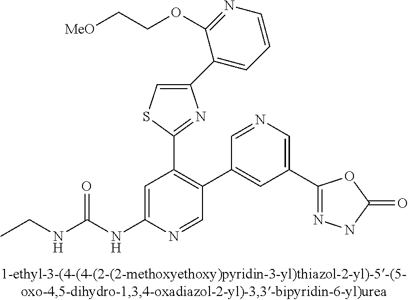 | LC/MS (ES⁺)[(M + H)⁺]: 561 for C₂₆H₂₄N₈O₅S. ¹H NMR (300 MHz, d₆-DMSO): 1.11 (t, 3H), 3.22 (m, 2H), 3.31 (s, 3H), 3.76 (t, 2H), 4.52 (t, 2H), 7.02 (m, 1H), 7.64 (m, 1H), 8.01 (d, 1H), 8.13 (d, 1H), 8.20 (s, 1H), 8.26 (s, 2H), 8.34 (s, 1H), 8.68 (s, 1H), 8.98 (s, 1H), 9.49 (s, 1H), 12.63 (s, 1H). | Intermediate 371 |
| 209 | 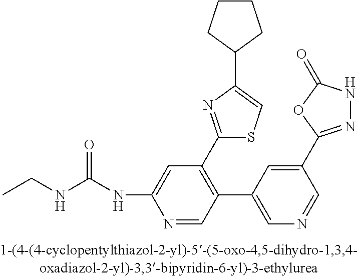 | LC/MS (ES⁺)[(M + H)⁺]: 478 for C₂₃H₂₃N₇O₃S. ¹H NMR (300 MHz, d₆-DMSO): 1.11 (t, 3H), 1.37 (m, 2H), 1.50 (m, 4H), 1.79 (m, 2H), 3.05 (m, 1H), 3.21 (m, 2H), 7.41 (s, 1H), 7.66 (m, 1H), 8.01 (m, 1H), 8.09 (s, 1H), 8.30 (s, 1H), 8.60 (d, 1H), 8.93 (d, 1H), 9.45 (s, 1H), 12.77 (s, 1H). | Intermediate 375 |
| 210 | 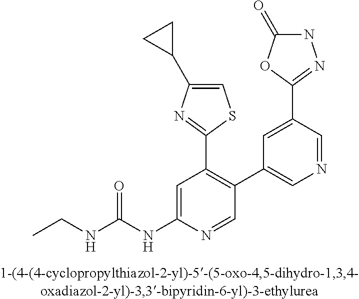 | LC/MS (ES⁺)[(M + H)⁺]: 450 for C₂₁H₁₉N₇O₃S. ¹H NMR (300 MHz, d₆-DMSO): 0.45 (m, 2H), 0.76 (m, 2H), 1.10 (t, 3H), 1.97 (m, 1H), 3.21 (m, 2H), 7.40 (s, 1H), 7.63 (m, 1H), 8.02 (m, 1H), 8.08 (s, 1H), 8.27 (s, 1H), 8.55 (d, 1H), 8.95 (d, 1H), 9.41 (s, 1H), 12.74 (s, 1H). | Intermediate 376 |
| 211 | 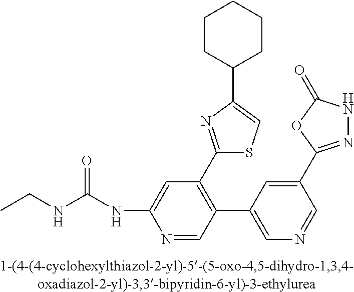 | LC/MS (ES⁺)[(M + H)⁺]: 492 for C₂₄H₂₅N₇O₃S. ¹H NMR (300 MHz, d₆-DMSO): 1.11 (t, 3H), 1.14 (m, 2H), 1.23 (m, 3H), 1.61 (m, 3H), 1.71 (m, 2H), 2.57 (m, 1H), 3.21 (m, 2H), 7.37 (s, 1H), 7.65 (m, 1H), 8.04 (m, 1H), 8.11 (s, 1H), 8.30 (s, 1H), 8.61 (d, 1H), 8.94 (d, 1H), 9.44 (s, 1H), 12.74 (s, 1H). | Intermediate 374 |
| 212 | 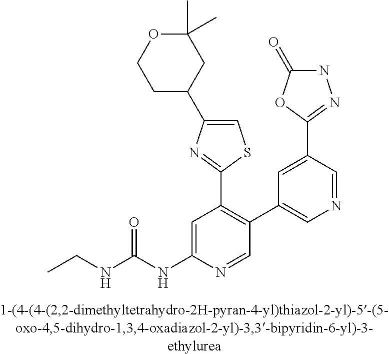 | LC/MS (ES⁺)[(M + H)⁺]: 522 for C₂₅H₂₇N₇O₄S. ¹H NMR (300 MHz, d₆-DMSO): 1.05 (s, 3H), 1.11 (t, 3H), 1.13 (s, 3H), 1.14 (m, 1H), 1.33 (m, 1H), 1.45 (m, 1H), 1.61 (m, 1H), 2.98 (m, 1H), 3.21 (m, 2H), 3.59 (m, 2H), 7.43 (s, 1H), 7.63 (m, 1H), 7.99 (m, 1H), 8.10 (s, 1H), 8.31 (s, 1H), 8.61 (d, 1H), 8.93 (d, 1H), 9.43 (s, 1H), 12.78 (s, 1H). | Intermediate 373 |
| 213 | 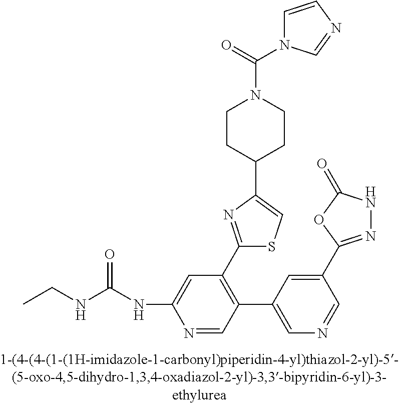 | LC/MS (ES⁺)[(M + H)⁺]: 587 for C₂₇H₂₆N₁₀O₄S. ¹H NMR (300 MHz, d₆-DMSO): 1.11 (t, 3H), 1.52 (m, 2H), 1.83 (m, 2H), 2.79 (m, 1H), 3.11 (m, 1H), 3.21 (m, 2H), 3.83 (m, 2H), 7.12 (s, 1H), 7.49 (m, 1H), 7.50 (s, 1H), 7.60 (m, 1H), 7.70 (m, 1H), 8.04 (m, 1H), 8.14 (s, 1H), 8.16 (s, 1H), 8.32 (s, 1H), 8.61 (d, 1H), 8.92 (d, 1H), 9.43 (s, 1H), 12.77 (s, 1H). | Intermediate 377 |
| 214 | 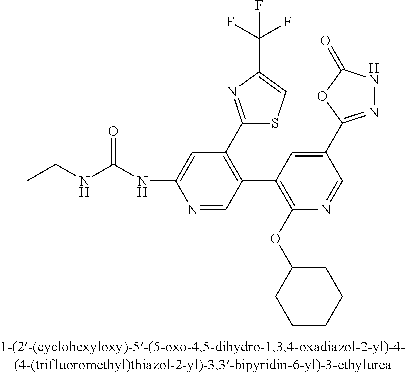 | LC/MS (ES⁺)[(M + H)⁺]: 576 for C₂₅H₂₄F₃N₇O₄S. ¹H NMR (300 MHz, d₆-DMSO): 1.07 (m, 2H), 1.11 (t, 3H), 1.17 (m, 2H), 1.35 (m, 4H), 1.52 (m, 2H), 3.21 (m, 2H), 4.87 (m, 1H), 7.60 (m, 1H), 8.16 (d, 1H), 8.19 (s, 1H), 8.26 (s, 1H), 8.53 (s, 1H), 8.62 (d, 1H), 9.47 (s, 1H), 12.65 (s, 1H). | Intermediate 378 |
| 215 | 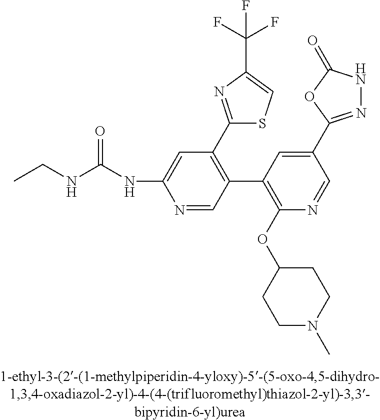 | LC/MS (ES⁺)[(M + H)⁺]: 591 for C₂₅H₂₅F₃N₈O₄S. ¹H NMR (300 MHz, d₆-DMSO): 1.11 (t, 3H), 1.17 (m, 2H), 1.57 (m, 2H), 2.0 (m, 2H), 2.02 (s, 3H), 2.12 (m, 2H), 3.21 (m, 2H), 4.87 (m, 1H), 7.61 (m, 1H), 8.15 (d, 1H), 8.23 (s, 1H), 8.26 (s, 1H), 8.54 (s, 1H), 8.62 (d, 1H), 9.48 (s, 1H), 12.61 (s, 1H). | Intermediate 380 |
| 216 | 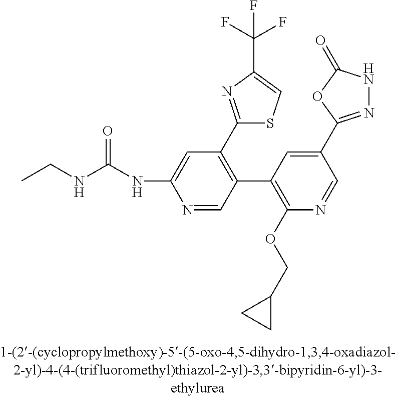 | LC/MS (ES⁺)[(M + H)⁺]: 548 for C₂₃H₂₀F₃N₇O₄S. ¹H NMR (300 MHz, d₆-DMSO): 0.04 (m, 2H), 0.26 (m, 2H), 0.81 (m, 1H), 1.11 (t, 3H), 3.21 (m, 2H), 3.88 (m, 2H), 7.58 (m, 1H), 8.15 (m, 1H), 8.24 (s, 1H), 8.28 (s, 1H), 8.54 (s, 1H), 8.63 (d, 1H), 9.47 (s, 1H), 12.68 (s, 1H). | Intermediate 383 |
| 217 | 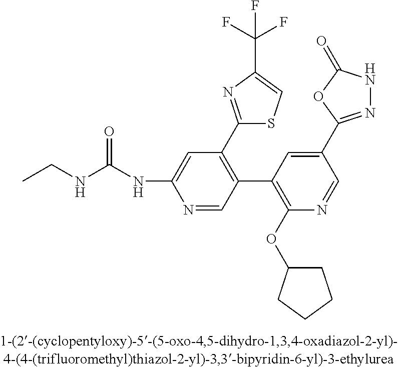 | LC/MS (ES⁺)[(M + H)⁺]: 562 for C₂₄H₂₂F₃N₇O₄S. ¹H NMR (300 MHz, d₆-DMSO): 1.11 (t, 3H), 1.20 (m, 2H), 1.23 (m, 2H), 1.34 (m, 2H), 1.66 (m, 2H), 3.21 (m, 2H), 5.18 (m, 1H), 7.61 (m, 1H), 8.12 (d, 1H), 8.20 (s, 1H), 8.25 (s, 1H), 8.53 (s, 1H), 8.60 (d, 1H), 9.47 (s, 1H), 12.66 (s, 1H). | Intermediate 379 |
| 218 | 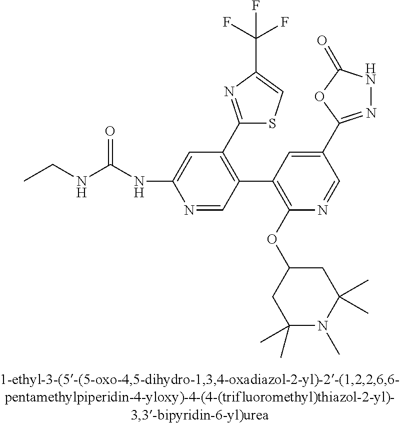 | LC/MS (ES⁺)[(M + H)⁺]: 647 for C₂₉H₃₃F₃N₈O₄S. ¹H NMR (300 MHz, d₆-DMSO): 1.11 (t, 3H), 1.23 (s, 2H), 1.25 (m, 2H), 1.30 (s, 12H), 1.42 (m, 2H), 2.65 (s, 3H), 5.27 (m, 1H), 7.52 (m, 1H), 8.18 (m, 1H), 8.28 (s, 1H), 8.59 (s, 1H), 8.61 (s, 1H), 8.9 (s, 1H), 9.5 (s, 1H), 12.7 (s, 1H). | Intermediate 382 |
| 219 | 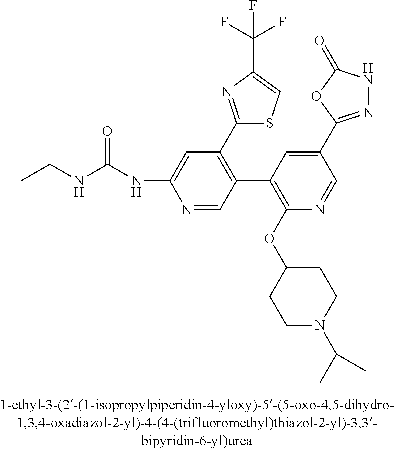 | LC/MS (ES⁺)[(M + H)⁺]: 619 for C₂₇H₂₉F₃N₈O₄S. ¹H NMR (300 MHz, d₆-DMSO): 0.85 (d, 6H), 1.10 (t, 3H), 1.14 (m, 2H), 1.60 (m, 2H), 2.27 (m, 4H), 2.64 (m, 1H), 3.22 (m, 2H), 4.94 (m, 1H), 7.63 (m, 1H), 8.15 (d, 1H), 8.23 (s, 1H), 8.26 (s, 1H), 8.53 (s, 1H), 8.63 (d, 1H), 9.47 (s, 1H), 12.33 (s, 1H). | Intermediate 381 |
| 220 | 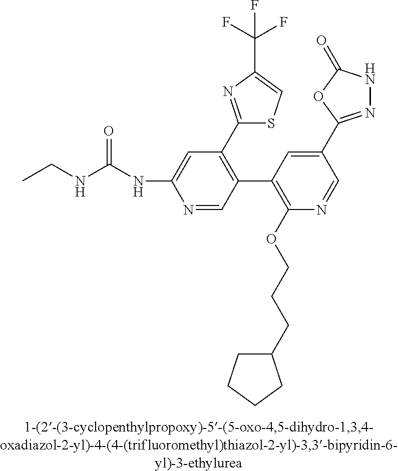 | LC/MS (ES⁺)[(M + H)⁺]: 604 for C₂₇H₂₈F₃N₇O₄S. ¹H NMR (300 MHz, d₆-DMSO): 0.86 (m, 2H), 1.00 (m, 2H), 1.11 (t, 3H), 1.27 (m, 2H), 1.41 (m, 2H), 1.45 (m, 2H), 1.54 (m, 3H), 3.21 (m, 2H), 4.03 (m, 2H), 7.61 (m, 1H), 8.12 (d, 1H), 8.26 (s, 2H), 8.53 (s, 1H), 8.64 (d, 1H), 9.45 (s, 1H), 12.63 (s, 1H). | Intermediate 384 |
| 221 | 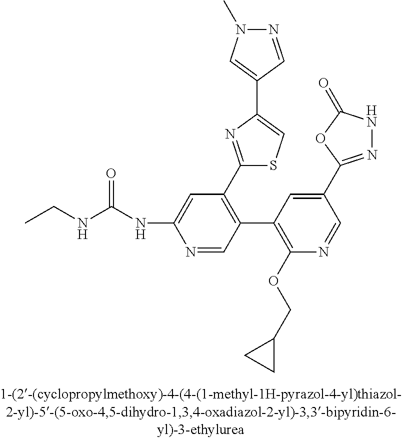 | LC/MS (ES⁺)[(M + H)⁺]: 560 for C₂₆H₂₅N₉O₄S. ¹H NMR (300 MHz, d₆-DMSO): 0.08 (m, 2H), 0.23 (m, 2H), .79 (m, 1H), 1.11 (t, 3H), 3.22 (m, 2H), 3.82 (m, 2H), 3.84 (s, 3H), 7.56 (s, 1H), 7.66 (m, 1H), 7.73 (s, 1H), 7.88 (s, 1H), 8.08 (m, 1H), 8.21 (s, 1H), 8.23 (s, 1H), 8.62 (d, 1H), 9.43 (s, 1H), 12.61 (s, 1H). | Intermediate 367 |
| 222 | 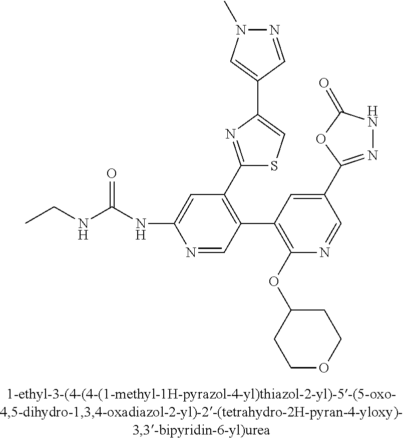 | LC/MS (ES⁺)[(M + H)⁺]: 590 for C₂₇H₂₇N₉O₅S. ¹H NMR (300 MHz, d₆-DMSO): 0.9 (m, 2H), 1.11 (t, 3H), 1.14 (m, 2H), 1.6 (m, 2H), 3.22 (m, 2H), 3.25 (m, 2H), 3.83 (s, 3H), 5.05 (m, 1H), 7.53 (s, 1H), 7.68 (m, 1H), 7.73 (s, 1H), 7.87 (s, 1H), 8.11 (m, 1H), 8.19 (s, 1H), 8.23 (s, 1H), 8.63 (d, 1H), 9.44 (s, 1H), 12.60 (s, 1H). | Intermediate 366 |
| 223 | 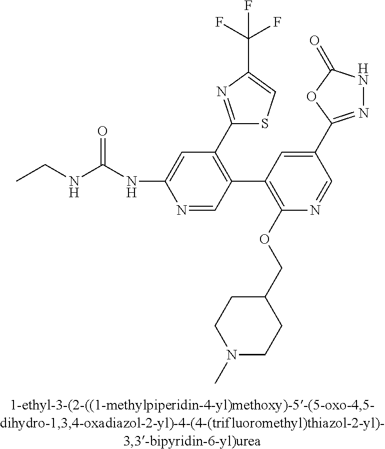 | LC/MS (ES⁺)[(M + H)⁺]: 605 for C₂₆H₂₇F₃N₈O₄S. ¹H NMR (300 MHz, d₆-DMSO): 0.94 (m, 2H), 1.11 (t, 3H), 1.23 (m, 3H), 1.70 (m, 2H), 2.07 (s, 3H), 2.58 (m, 2H), 3.21 (m, 2H), 3.92 (m, 2H), 7.58 (m, 1H), 8.13 (d, 1H), 8.26 (s, 1H), 8.28 (s, 1H), 8.52 (s, 1H), 8.65 (d, 1H), 9.44 (s, 1H), 12.41 (s, 1H). | Intermediate 386 |
| 224 | 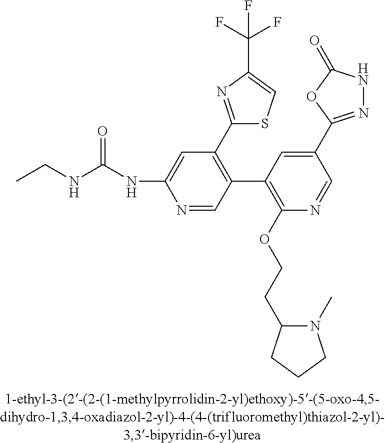 | LC/MS (ES⁺)[(M + H)⁺]: 605 for C₂₆H₂₇F₃N₈O₄S. ¹H NMR (300 MHz, d₆-DMSO): 1.11 (t, 3H), 1.17 (m, 2H), 1.48 (m, 4H), 1.78 (m, 1H), 1.92 (m, 1H), 2.04 (s, 3H), 2.84 (m, 1H), 3.21 (m, 2H), 4.06 (m, 2H), 7.59 (m, 1H), 8.13 (d, 1H), 8.25 (s, 1H), 8.26 (s, 1H), 8.54 (s, 1H), 8.64 (d, 1H), 9.44 (s, 1H), 12.54 (s, 1H). | Intermediate 385 |
| 225 | 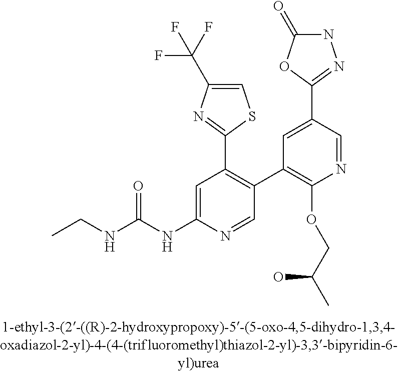 | LC/MS (ES⁺)[(M + H)⁺]: 552 for C₂₂H₂₀F₃N₇O₅S. ¹H NMR (300 MHz, d₆-DMSO): 0.75 (d, 3H), 1.11 (t, 3H), 3.21 (m, 2H), 3.53 (m, 1H), 3.70 (m, 1H), 4.02 (m, 1H), 4.61 (d, 1H), 7.58 (m, 1H), 8.12 (s, 1H), 8.25 (s, 1H), 8.28 (s, 1H), 8.52 (s, 1H), 8.64 (s, 1H), 9.45 (s, 1H), 12.63 (s, 1H). | Intermediate 390 |
| 226 | 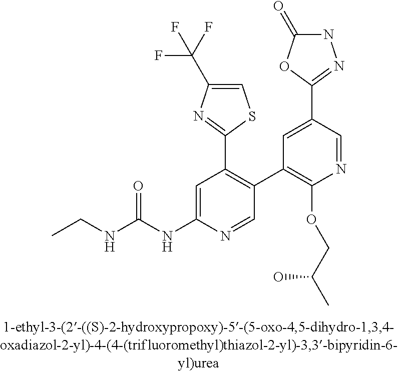 | LC/MS (ES⁺)[(M + H)⁺]: 552 for C₂₂H₂₀F₃N₇O₅S. ¹H NMR (300 MHz, d₆-DMSO): 0.76 (d, 3H), 1.11 (t, 3H), 3.21 (m, 2H), 3.53 (m, 1H), 3.71 (m, 1H), 4.02 (m, 1H), 4.60 (d, 1H), 7.58 (t, 1H), 8.11 (s, 1H), 8.25 (s, 1H), 8.28 (s, 1H), 8.51 (s, 1H), 8.64 (s, 1H), 9.44 (s, 1H), 12.63 (s, 1H). | Intermediate 391 |
| 227 | 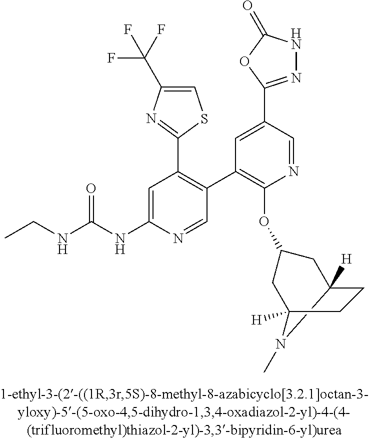 | LC/MS (ES⁺)[(M + H)⁺]: 617 for C₂₇H₂₇F₃N₈O₄S. ¹H NMR (400 MHz, d₆-DMSO): 1.11 (t, 3H), 1.17 (m, 2H), 1.24 (s, 3H), 1.35 (m, 2H), 1.81 (m, 2H), 2.25 (m, 2H), 3.06 (m, 2H), 3.21 (m, 1H), 3.61 (m, 1H), 5.17 (m, 1H), 7.50 (m, 1H), 8.19 (m, 1H), 8.29 (s, 1H), 8.34 (s, 1H), 8.54 (s, 1H), 8.66 (s, 1H), 9.46 (s, 1H), 12.63 (s, 1H). | Intermediate 414 |

### Examples 228-230

The following Examples were prepared as described for Example 1 using the starting materials as indicated in the table.

| Ex | Compound | Data | SM |
|---|---|---|---|
| 228 | 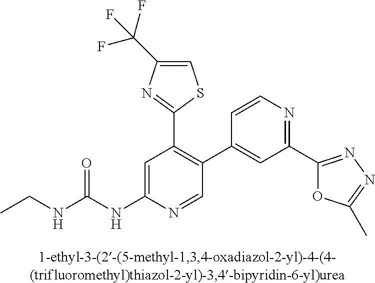 | LC/MS (ES⁺)[M + H)⁺]: 476 for C₂₀H₁₆F₃N₇O₂S. ¹H NMR (300 MHz, d₆-DMSO): 1.11 (t, 3H), 2.61 (s, 3H), 3.19 (m, 2H), 7.55 (m, 2H), 8.01 (s, 1H), 8.18 (s, 1H), 8.42 (s, 1H), 8.61 (s, 1H), 8.74 (m, 1H), 9.57 (s, 1H). | Intermediate 399 |
| 229 | 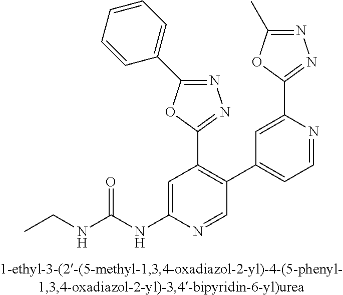 | LC/MS (ES⁺)[M + H)⁺]: 469 for C₂₄H₂₀N₈O₃. ¹H NMR (300 MHz, d₆-DMSO): 1.05 (t, 3H), 2.54 (s, 3H), 3.16 (m, 2H), 7.41 (t, 1H), 7.50 (m, 3H), 7.65 (m, 3H), 8.16 (s, 1H), 8.39 (s, 2H), 8.73 (d, 1H), 9.56 (s, 1H). | Intermediate 400 |
| 230 | 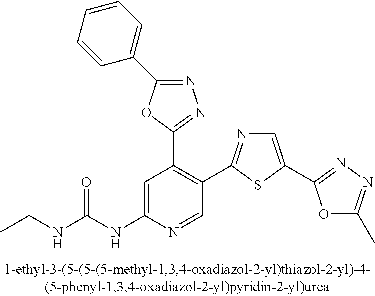 | LC/MS (ES⁺)[M + H)⁺]: 475 for C₂₂H₁₈N₈O₃S. ¹H NMR (300 MHz, d₆-DMSO): 1.05 (t, 3H), 2.51 (s, 3H), 3.15 (m, 2H), 7.39 (t, 1H), 7.53 (m, 3H), 7.78 (m, 2H), 8.24 (s, 1H), 8.40 (s, 1H), 8.77 (s, 1H), 9.75 (s, 1H). | Intermediate 401 |

### Example 231

### 1-ethyl-3-(5′-(5-methyl-1,3,4-oxadiazol-2-yl)-4-(4-(1-methyl-1H-pyrazol-4-yl)thiazol-2-yl)-2′-(tetrahydro-2H-pyran-4-yloxy)-3,3′-bipyridin-6-yl)urea

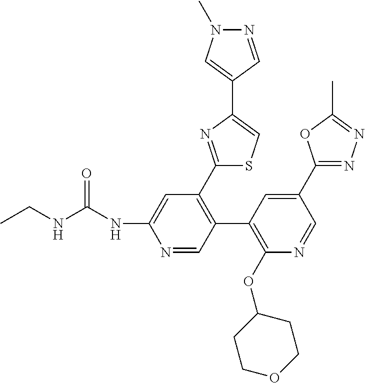

In a 25 mL pear flask, 1-ethyl-3-(5′-(hydrazinecarbonyl)-4-(4-(1-methyl-1H-pyrazol-4-yl)thiazol-2-yl)-2′-(tetrahydro-2H-pyran-4-yloxy)-3,3′-bipyridin-6-yl)urea (Intermediate 366, 68.1 mg, 0.12 mmol) and 1,1,1-trimethoxyethane (461 μl, 3.62 mmol) were suspended in solvent. The reaction slurry was heated to reflux for 30 min. 2,3,4,6,7,8,9,10-octahydropyrimido[1,2-a]azepine (18.07 μl, 0.12 mmol) was added in a single portion. The reaction was heated for an additional for 2 h. The reaction mixture was cooled to room temperature, diluted with EtOAc and washed with water then brine. The organic phase was dried over Na₂SO₄, filtered and concentrated by rotary evaporation. The crude was purified by silica gel flash column chromatography (95:5 CH₂Cl₂/MeOH). Isolation gave 40 mg of the desired product.

LC/MS (ES⁺)[(M+H)^{+]}: 588 for C₂₈H₂₉N₉O₄S.

¹H NMR (300 MHz, d₆-DMSO): 1.12 (t, 3H), 1.16 (m, 2H), 1.60 (m, 2H), 2.59 (s, 3H), 3.22 (m, 2H), 3.25 (m, 2H), 3.37 (m, 2H), 3.83 (s, 3H), 5.08 (m, 1H), 7.50 (s, 1H), 7.67 (m, 1H), 7.72 (s, 1H), 7.88 (s, 1H), 8.21 (s, 1H), 8.26 (s, 1H), 8.27 (m, 1H), 8.81 (m, 1H), 9.45 (s, 1H).

### Examples 232-236

The following Examples were prepared according to the procedure as described by Example 231 using the starting materials as indicated.

| Ex | Compound | Data | SM |
|---|---|---|---|
| 232 | 1-ethyl-3-(4-(4-(2-(2-methoxyethoxy)pyridin- 3-yl)thiazol-2-yl)-5′-(5-methyl-1,3,4- oxadiazol-2-yl)-2′-(tetrahydro-2H-pyran- 4-yloxy)-3,3′-bipyridin-6-yl)urea 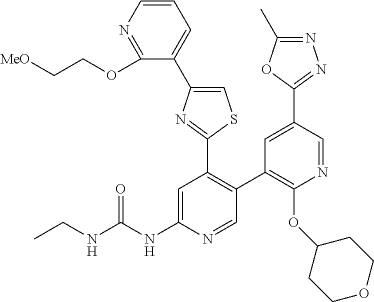 | MS (ES⁺)[(M + H)⁺]: 658 for C₃₂H₃₄N₈O₆S. ¹H NMR (300 MHz, d₆-DMSO): 0.87 (m, 2H), 1.12 (t, 3H), 1.28 (m, 2H), 1.76 (m, 2H), 2.60 (s, 3H), 2.64 (m, 2H), 3.24 (m, 2H), 3.31 (s, 3H), 3.75 (t, 2H), 4.52 (t, 2H), 5.06 (m, 1H), 6.99 (m, 1H), 7.69 (m, 1H), 7.83 (m, 1H), 8.11 (m, 1H), 8.22 (s, 1H), 8.27 (s, 1H), 8.29 (s, 1H), 8.30 (d, 1H), 8.81 (m, 1H), 9.47 (s, 1H). | Intermediate 372 |
| 233 | 1-ethyl-3-(5′-(5-methyl-1,3,4- oxadiazol-2-yl)-4-(4-(1-methyl-1H-pyrazol- 4-yl)thiazol-2-yl)-3,3′-bipyridin-6-yl)urea 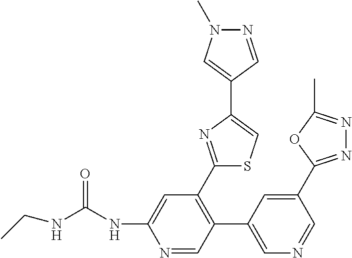 | LC/MS (ES⁺)[(M + H)⁺]: 488 for C₂₃H₂₁N₉O₂S. ¹H NMR (300 MHz, d₆-DMSO): 1.11 (t, 3H), 2.58 (s, 3H), 3.20 (m, 2H), 3.82 (s, 3H), 7.61 (s, 1H), 7.62 (m, 1H), 7.77 (s, 1H), 7.92 (s, 1H), 8.20 (s, 1H), 8.33 (s, 1H), 8.34 (s, 1H), 8.70 (m, 1H), 9.15 (m, 1H), 9.48 (s, 1H). | Intermediate 36 |
| 234 | 1-(2′-(cyclopropylmethoxy)-5′-(5-methyl-1,3,4- oxadiazol-2-yl)-4-(4-(trifluoromethyl)thiazol- 2-yl)-3,3′-bipyridin-6-yl)-3-ethylurea 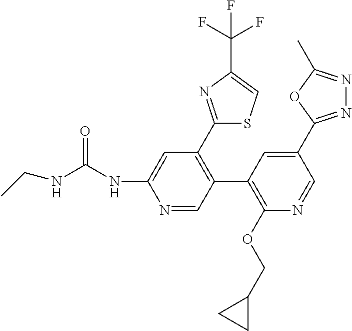 | LC/MS (ES⁺)[(M + H)⁺]: 546 for C₂₄H₂₂F₃N₇O₃S. ¹H NMR (300 MHz, d₆-DMSO): 0.04 (m, 2H), 0.28 (m, 2H), 0.82 (m, 1H), 1.11 (t, 3H), 2.59 (s, 3H), 3.21 (m, 2H), 3.88 (d, 2H), 7.57 (m, 1H), 8.25 (s, 1H), 8.30 (m, 1H), 8.31 (s, 1H), 8.54 (s, 1H), 8.81 (d, 1H), 9.48 (s, 1H). | Intermediate 383 |
| 235 | 1-(4-(4-cyclopentylthiazol-2- yl)-5′-(5-methyl-1,3,4-oxadiazol-2-yl)-3,3′- bipyridin-6-yl)-3-ethylurea 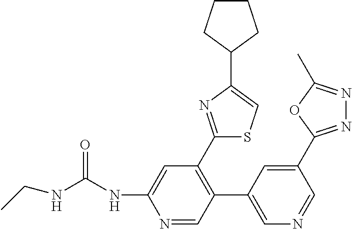 | LC/MS (ES⁺)[(M + H)⁺]: 476 for C₂₄H₂₅N₇O₂S. ¹H NMR (300 MHz, d₆-DMSO): 1.11 (t, 3H), 1.36 (m, 2H), 1.46 (m, 4H), 1.74 (m, 2H), 2.59 (s, 3H), 3.03 (m, 1H), 3.21 (m, 2H), 7.40 (s, 1H), 7.66 (m, 1H), 8.11 (s, 1H), 8.19 (m, 1H), 8.33 (s, 1H), 8.64 (d, 1H), 9.11 (d, 1H), 9.49 (s, 1H). | Intermediate 375 |
| 236 | 1-(4-(4-cyclohexylthiazol-2- yl)-5′-(5-methyl-1,3,4-oxadiazol-2-yl)-3,3′- bipyridin-6-yl)-3-ethylurea 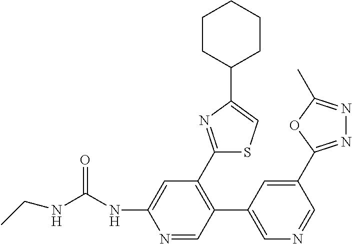 | LC/MS (ES⁺)[(M + H)⁺]: 490 for C₂₅H₂₇N₇O₂S. ¹H NMR (300 MHz, d₆-DMSO): 1.11 (t, 3H), 1.16 (m, 5H), 1.58 (m, 3H), 1.72 (m, 2H), 2.57 (m, 1H), 2.59 (s, 3H), 3.21 (m, 2H), 7.38 (s, 1H), 7.67 (m, 1H), 8.14 (s, 1H), 8.22 (m, 1H), 8.32 (s, 1H), 8.65 (d, 1H), 9.12 (d, 1H), 9.47 (s, 1H). | Intermediate 374 |

### Example 237

### (S)-1-ethyl-3-(5′-(5-(1-hydroxyethyl)-1,3,4-oxadiazol-2-yl)-4-(4-(trifluoromethyl)thiazol-2-yl)-3,3′-bipyridin-6-yl)urea

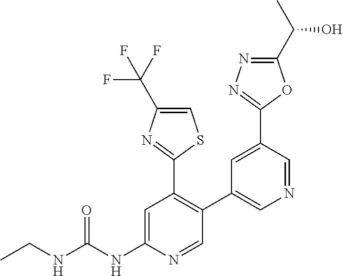

In a glass vial, (S)-1-ethyl-3-(5′-(2-(2-(triethylsilyloxy)propanoyl)hydrazinecarbonyl)-4-(4-(trifluoromethyl)thiazol-2-yl)-3,3′-bipyridin-6-yl)urea (Intermediate 403, 200 mg, 0.31 mmol) was suspended in a acetonitrile solution containing carbon tetrachloride (0.091 mL, 0.94 mmol) and diisopropylethyl amine (0.168 mL, 0.94 mmol). Triphenylphosphine (247 mg, 0.94 mmol) was added in a single portion. The reaction mixture was gently warmed to form a homogenous solution and was then allowed to stir at room temperature overnight. Once cyclized, the reaction mixture was acidified to pH=1 with 6N HCl. The reaction mixture was diluted with EtOAc, washed with NaHCO₃ (sat.) then brine. Dried the organic phase over Na₂SO₄, filtered and concentrated to dryness by rotary evaporation. Purified by silica gel flash column chromatography (95:5 CH₂Cl₂/MeOH). Isolated 72 mg of the title compound.

LC/MS (ES⁺)[(M+H)⁺]: 506 for C₂₁H₁₈F₃N₇O₃S.

¹H NMR (300 MHz, d₆-DMSO): 1.12 (t, 3H), 1.53 (d, 3H), 3.22 (m, 2H), 5.02 (m, 1H), 6.03 (d, 1H), 7.55 (t, 1H), 8.25 (s, 1H), 8.33 (t, 1H), 8.42 (s, 1H), 8.57 (s, 1H), 8.72 (d, 1H), 9.20 (d, 1H), 9.50 (s, 1H).

### Example 238

### (S)-1-(5′-(5-(amino(cyclohexyl)methyl)-1,3,4-oxadiazol-2-yl)-4-(4-(trifluoromethyl)thiazol-2-yl)-3,3′-bipyridin-6-yl)-3-ethylurea

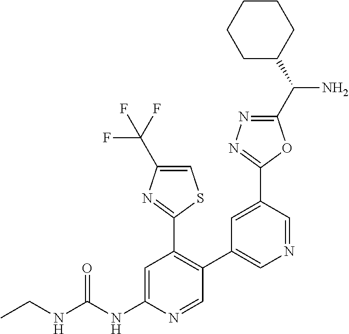

(S)-tert-butyl cyclohexyl(5-(6′-(3-ethylureido)-4′-(4-(trifluoromethyl)thiazol-2-yl)-3,3′-bipyridin-5-yl)-1,3,4-oxadiazol-2-yl)methylcarbamate (Intermediate 404, 100 mg, 0.15 mmol) was dissolved in 1,4 dioxane. 4N HCl in dioxane (4 mL, 16.00 mmol) was added in a single portion. The solution was stirred at room temperature for 12 h. Concentrate the reaction mixture to dryness by rotary evaporation. Dissolve the crude in EtOAc, washed with 10% NaHCO₃, dried over Na₂SO₄, filtered, concentrated and purified by silica gel flash column chromatography (95:5 CH₂Cl₂/MeOH). Isolation gave 63 mg of the title compound.

LC/MS (ES⁺)[(M+H)⁺]: 573 for C₂₆H₂₇F₃N₈O₂S.

¹H NMR (300 MHz, d₆-DMSO): 1.01-1.21 (m, 5H), 1.12 (t, 3H), 1.44 (m, 1H), 1.69 (m, 4H), 1.85 (m, 1H), 2.16 (s, 2H), 3.22 (m, 2H), 3.88 (d, 1H), 7.55 (t, 1H), 8.24 (s, 1H), 8.30 (t, 1H), 8.42 (s, 1H), 8.57 (s, 1H), 8.72 (d, 1H), 9.20 (d, 1H), 9.51 (s, 1H).

### Examples 239-244

The following Examples were prepared according to the procedures described for Example 238 using the starting materials indicated in the table.

| Ex | Compound | Data | SM |
|---|---|---|---|
| 239 | (S)-1-ethyl-3-(5′-(5-(morpholin-3-yl)-1,3,4- oxadiazol-2-yl)-4-(4-(trifluoromethyl)thiazol- 2-yl)-3,3′-bipyridin-6-yl)urea 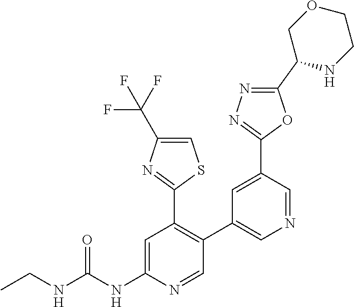 | LC/MS (ES⁺)[(M + H)⁺]: 547 for C₂₃H₂₁F₃N₈O₃S. ¹H NMR (300 MHz, d₆-DMSO): 1.11 (t, 3H), 2.82 (m, 1H), 2.94 (m, 1H), 3.14 (m, 2H), 3.23 (s, 1H), 3.54 (m, 1H), 3.65 (m, 1H), 3.78 (m, 1H), 3.96 (dd, 1H), 4.34 (dd, 1H), 7.48 (t, 1H), 8.19 (s, 1H), 8.29 (t, 1H), 8.34 (s, 1H), 8.50 (s, 1H), 8.65 (d, 1H), 9.15 (d, 1H), 9.44 (s, 1H). | Intermediate 405 |
| 240 | (R)-1-ethyl-3-(5′-(5-(morpholin-3-yl)-1,3,4- oxadiazol-2-yl)-4-(4-(trifluoromethyl)thiazol- 2-yl)-3,3′-bipyridin-6-yl)urea 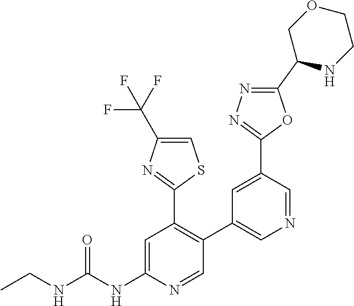 | LC/MS (ES⁺)[(M + H)⁺]: 547 for C₂₃H₂₁F₃N₈O₃S. ¹H NMR (300 MHz, d₆-DMSO): 1.11 (t, 3H), 2.82 (m, 1H), 2.94 (m, 1H), 3.14 (m, 2H), 3.23 (s, 1H), 3.54 (m, 1H), 3.65 (m, 1H), 3.78 (m, 1H), 3.96 (dd, 1H), 4.34 (dd, 1H), 7.48 (t, 1H), 8.19 (s, 1H), 8.29 (t, 1H), 8.34 (s, 1H), 8.50 (s, 1H), 8.65 (d, 1H), 9.15 (d, 1H), 9.44 (s, 1H). | Intermediate 406 |
| 241 | (S)-1-(2′-(5-(amino(cyclohexyl)methyl)- 1,3,4-oxadiazol-2-yl)-4-(4-(trifluoromethyl) thiazol-2-yl)-3,4′-bipyridin-6-yl)-3-ethylurea 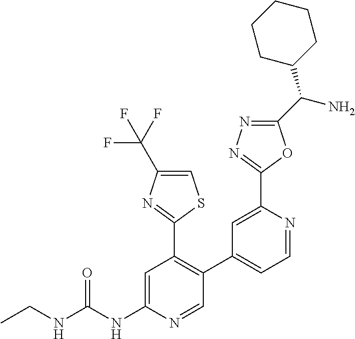 | LC/MS (ES⁺)[(M + H)⁺]: 573 for C₂₆H₂₇F₃N₈O₂S. ¹H NMR (300 MHz, d₆-DMSO): 1.02-1.21 (m, 5H), 1.11 (t, 3H), 1.43 (m, 1H), 1.69 (m, 4H), 1.85 (m, 1H), 2.15 (m, 2H), 3.22 (m, 2H), 3.90 (d, 1H), 7.53 (t, 1H), 7.56 (dd, 1H), 8.01 (d, 1H), 8.18 (s, 1H), 8.43 (s, 1H), 8.60 (d, 1H), 8.76 (d, 1H), 9.54 (s, 1H). | Intermediate 407 |
| 242 | (S)-1-ethyl-3-(2′-(5-(morpholin-3-yl)-1,3,4- oxadiazol-2-yl)-4-(4-(trifluoromethyl)thiazol- 2-yl)-3,4′-bipyridin-6-yl)urea 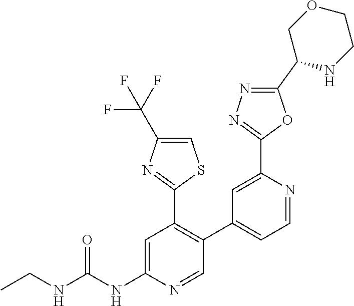 | LC/MS (ES⁺)[(M + H)⁺]: 547 for C₂₃H₂₁F₃N₈O₃S. ¹H NMR (300 MHz, d₆-DMSO): 1.11 (t, 3H), 2.80 (m, 1H), 2.92 (m, 1H), 3.21 (m, 3H), 3.56 (m, 1H), 3.66 (m, 1H), 3.79 (m, 1H), 3.94 (m, 1H), 4.25 (m, 1H), 7.53 (t, 1H), 7.56 (dd, 1H), 8.06 (d, 1H), 8.19 (s, 1H), 8.42 (s, 1H), 8.60 (s, 1H), 8.76 (d, 1H), 9.54 (s, 1H). | Intermediate 408 |
| 243 | (R)-1-ethyl-3-(2′-(5-(morpholin-3-yl)-1,3,4- oxadiazol-2-yl)-4-(4-(trifluoromethyl)thiazol- 2-yl)-3,4′-bipyridin-6-yl)urea 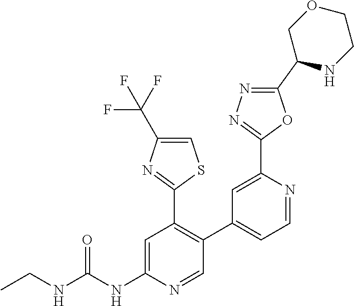 | LC/MS (ES⁺)[(M + H)⁺]: 547 for C₂₃H₂₁F₃N₈O₃S. ¹H NMR (300 MHz, d₆-DMSO): 1.11 (t, 3H), 2.80 (m, 1H), 2.92 (m, 1H), 3.21 (m, 3H), 3.56 (m, 1H), 3.66 (m, 1H), 3.79 (m, 1H), 3.94 (m, 1H), 4.25 (m, 1H), 7.53 (t, 1H), 7.56 (dd, 1H), 8.06 (d, 1H), 8.19 (s, 1H), 8.42 (s, 1H), 8.60 (s, 1H), 8.76 (d, 1H), 9.54 (s, 1H). | Intermediate 409 |
| 244 | (S)-1-ethyl-3-(2′-(5-(2-methyl-1- (methylamino)propyl)-1,3,4-oxadiazol-2-yl)- 4-(4-(trifluoromethyl)thiazol-2-yl)-3,4′- bipyridin-6-yl)urea 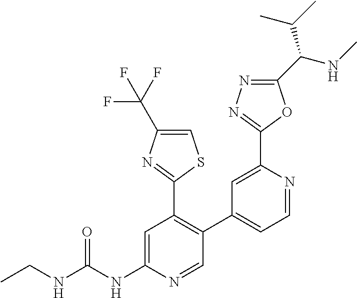 | LC/MS (ES⁺)[(M + H)⁺]: 547 for C₂₄H₂₅F₃N₈O₂S. ¹H NMR (300 MHz, d₆-DMSO): 0.83 (d, 3H), 0.99 (d, 3H), 1.11 (t, 3H), 2.09 (m, 1H), 2.21 (s, 3H), 2.31 (m, 1H), 3.22 (m, 2H), 3.63 (d, 1H), 7.54 (t, 1H), 7.58 (dd, 1H), 8.00 (s, 1H), 8.18 (s, 1H), 8.43 (s, 1H), 8.60 (s, 1H), 8.76 (d, 1H), 9.55 (s, 1H). | Intermediate 410 |

### Example 245 and Example 246

1-ethyl-3-(5-(3-(2-hydroxyethyl)-1,4-dioxo-1,2,3,4-tetrahydrophthalazin-6-yl)-4-(4-trifluoromethyl)thiazol-2-pyridin-2-yl)urea and 1-ethyl-3-(5-(2-(2-hydroxyethyl-1,4-dioxo-1,2,3,4-tetrahydrophthalazin-6-yl)-4-(4-(trifluoromethyl)thiazol-2-yl)pyridin-2-yl)urea

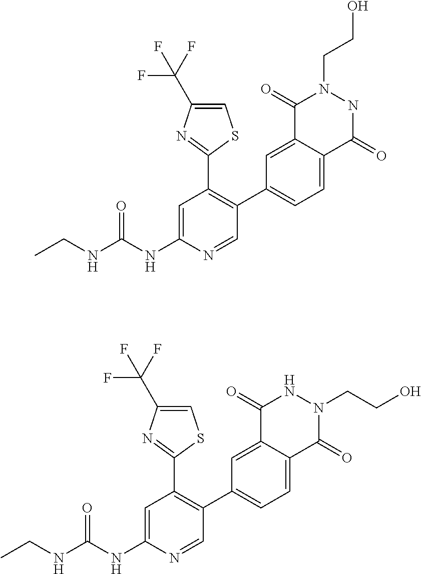

6-(3-ethylureido)-4-(4-(trifluoromethyl)thiazol-2-yl)pyridin-3-ylboronic acid (Intermediate 12, 400 mg, 1.11 mmol), a 1:1 mixture of 6-bromo-2-(2-hydroxyethyl)-2,3-dihydrophthalazine-1,4-dione and 7-bromo-2-(2-hydroxyethyl)-2,3-dihydrophthalazine-1,4-dione (Intermediates 411 and 412, 348 mg, 1.22 mmol), Pd(PPh₃)₄ (64.2 mg, 0.06 mmol) and cesium carbonate (543 mg, 1.67 mmol) were combined in a microwave vessel and suspended in a 4:1 mixture of dioxane and water. The reaction slurry was degassed and purged with nitrogen. The reaction mixture was heated in the microwave at 100° C. for 2 hours. The reaction mixture was concentrated to dryness by rotary evaporation. The residue was dissolved in minimal DMSO and water to help solubilize the inorganic salts. The two regioisomers were separated by reverse phase (C30 column) Gilson HPLC (10-50% MeOH/0.1% formic acid).

### Example 245

Isolated 38 mg. LC/MS (ES⁺)[(M+H)⁺]: 521 for C₂₂H₁₉F₃N₆O₄S. ¹H NMR (300 MHz, d₆-DMSO): 1.04 (t, 3H), 3.14 (m, 2H), 3.66 (t, 2H), 3.98 (t, 2H), 7.57 (t, 1H), 7.65 (d, 1H), 7.83 (s, 1H), 8.12 (d, 1H), 8.17 (s, 1H), 8.27 (s, 1H), 8.41 (s, 1H), 9.48 (s, 1H).

### Example 246

Isolated 37 mg. LC/MS (ES⁺)[(M+H)⁺]: 521 for C₂₂H₁₉F₃N₆O₄S. ¹H NMR (300 MHz, d₆-DMSO): 1.04 (t, 3H), 3.14 (m, 2H), 3.66 (t, 2H), 3.97 (t, 2H), 7.56 (t, 1H), 7.69 (dd, 1H), 7.92 (d, 1H), 8.06 (d, 1H), 8.17 (s, 1H), 8.28 (s, 1H), 8.42 (s, 1H), 9.45 (s, 1H).

### Example 247

### 6′-(3-ethylureido)-4′-(4-(6-methoxypyridin-2-yl)thiazol-2-yl)-3,3′-bipyridine-5-sulfonamide

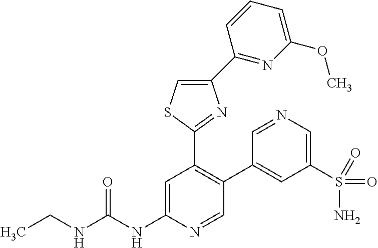

To a mixture of 6-(3-ethylureido)-4-(4-(6-methoxypyridin-2-yl)thiazol-2-yl)pyridin-3-ylboronic acid (Intermediate 415, 270 mg, 0.68 mmol), 5-bromopyridine-3-sulfonamide (192 mg, 0.81 mmol), Pd₂ dba₃ (31.0 mg, 0.03 mmol), dicyclohexyl(2′,4′,6′-triisopropylbiphenyl-2-yl)phosphine (97 mg, 0.20 mmol), and cesium carbonate (264 mg, 0.81 mmol) under vacuum, was added 1,4-dioxane (20 mL), water (5 mL), the reaction was then heated to 80° C. in an oil bath, purged with nitrogen, then stirred at that temperature under nitrogen pressure for 45 minutes. The reaction was then diluted with ethyl acetate (100 ml) and water (100 ml), then the layers were separated. The aqueous phase was extracted with ethyl acetate (3×100 ml), then the organics were combined, washed with brine, dried over sodium sulfate, filtered, concentrated under reduced pressure. The residue was suspended in methylene chloride with 5% methanol, loaded onto a silica gel column, eluted with a gradient of methanol in methylene chloride to give the desired product as a tan gum, which was suspended in dichloromethane and filtered to give the title compound as a pale tan solid (30 mg, 8.6%).

MS (EI) (M+H)⁺ 512 for C₂₂H₂₂N₇O₄S₂ (M−H)⁻ 510 for C₂₂H₂₀N₇O₄S₂

¹H NMR (DMSO-d6) δ: 9.51 (s, 1H), 8.98 (d, J=2.07 Hz, 1H), 8.73 (d, J=1.88 Hz, 1H), 8.34 (s, 1H), 8.32 (s, 1H), 8.29 (s, 1H), 8.18 (s, 1H), 7.73 (t, J=7.82 Hz, 1H), 7.66 (s, 1H), 7.58 (t, J=4.71 Hz, 1H), 7.22 (d, J=7.35 Hz, 1H), 6.78 (d, J=8.29 Hz, 1H), 3.92 (s, 3H), 3.22 (dq, J=6.88, 6.56 Hz, 2H), 1.11 (t, J=7.16 Hz, 3H)

### Example 248

### 1-ethyl-3-(4-(4-(6-methoxypyridin-2-yl)thiazol-2-yl)-2′-(5-oxo-4,5-dihydro-1,3,4-oxadiazol-2-yl)-3,4′-bipyridin-6-yl)urea

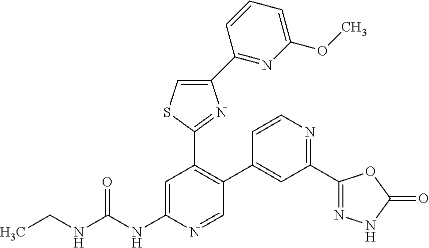

A solution of 1-ethyl-3-(2′-(hydrazinecarbonyl)-4-(4-(6-methoxypyridin-2-yl)thiazol-2-yl)-3,4′-bipyridin-6-yl)urea (Intermediate 416, 91 mg, 0.19 mmol) in DMF (2 mL) was treated with di(1H-imidazol-1-yl)methanone (60 mg, 0.37 mmol), warmed to 50° C. in an oil bath for 20 min, and cooled to room temperature. The reaction was held at room temperature for 1 hour, diluted with ethyl acetate (50 ml), washed with water (2×50 ml) and brine (30 ml). The organic layer was dried over magnesium sulfate, filtered and concentrated under reduced pressure to afford a tan mass. This material was suspended into acetonitrile (20 ml), warmed to reflux and cooled to give the title compound as a tan powder (70 mg, 73.1%).

MS (EI) (M+H)⁺ 517 for C₂₄H₂₁N₈O₄S (M−H)⁻ 515 for C₂₄H₁₉N₈O₄S

¹H NMR (DMSO-d6) δ: 12.77 (br. s., 1H), 9.52 (s, 1H), 8.70 (d, J=4.90 Hz, 1H), 8.35 (s, 1H), 8.35 (s, 1H), 8.22 (s, 1H), 7.90 (s, 1H), 7.70 (t, J=7.82 Hz, 1H), 7.58 (br. s., 1H), 7.54 (d, J=5.09 Hz, 1H), 7.20 (d, J=7.35 Hz, 1H), 6.78 (d, J=8.29 Hz, 1H), 3.91 (s, 3H), 3.22 (quin, J=6.69 Hz, 2H), 1.11 (t, J=7.16 Hz, 3H);

### Example 249

### 1-ethyl-3-(4-(4-(6-methoxypyridin-2-yl)thiazol-2-yl)-2′-(5-methyl-1,3,4-oxadiazol-2-yl)-3,4′-bipyridin-6-yl)urea

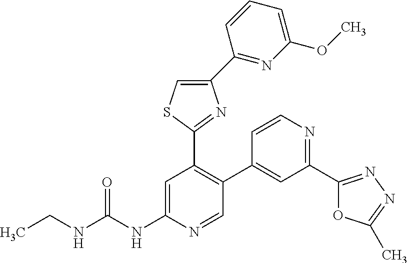

A suspension of 1-ethyl-3-(2′-(hydrazinecarbonyl)-4-(4-(6-methoxypyridin-2-yl)thiazol-2-yl)-3,4′-bipyridin-6-yl)urea (Intermediate 416, 80 mg, 0.16 mmol) in 1,1,1-trimethoxyethane (5 mL, 41.62 mmol) was treated with concentrated aqueous HCl (drop), and the resulting solution was heated to reflux for 5 minutes, the resulting pink solution was treated with DBU (0.1 mL, 0.66 mmol) and refluxed for an additional 30 minutes at which point solvent was removed and the residue purified by flash chromatography eluting with a gradient of methanol in methylene chloride to give the desired product as a tan solid (9 mg, 10.72%).

MS (EI) (M+H)⁺ 515 for C₂₅H₂₃N₈O₃S) (M−H)⁻ 513 for C₂₅H₂₁N₈O₃S

¹H NMR (DMSO-d6) δ: 9.48-9.64 (m, 1H), 8.75 (d, J=5.09 Hz, 1H), 8.38 (s, 1H), 8.35 (s, 1H), 8.23 (s, 1H), 8.11 (s, 1H), 7.66 (t, J=7.82 Hz, 1H), 7.54-7.63 (m, 2H), 7.18 (d, J=7.35 Hz, 1H), 6.76 (d, J=8.29 Hz, 1H), 3.90 (s, 3H), 3.12-3.25 (m, 2H), 2.59 (s, 3H), 1.11 (t, J=7.16 Hz, 3H);

### Example 250

### 1-ethyl-3-(4-(4-(6-methoxypyridin-2-yl)thiazol-2-yl)-5′-(5-methyl-1,3,4-oxadiazol-2-yl)-3,3′-bipyridin-6-yl)urea

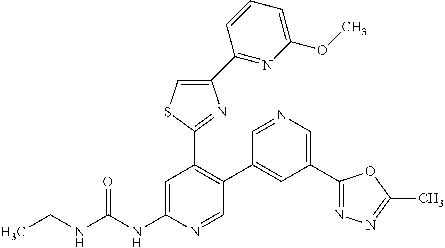

To a mixture of 6-(3-ethylureido)-4-(4-(6-methoxypyridin-2-yl)thiazol-2-yl)pyridin-3-ylboronic acid (Intermediate 415, 510 mg, 0.15 mmol), 2-(5-bromopyridin-3-yl)-5-methyl-1,3,4-oxadiazole (Intermediate 418, 36.8 mg, 0.15 mmol), dicyclohexyl(2′,4′,6′-triisopropylbiphenyl-2-yl)phosphine (21.92 mg, 0.05 mmol), Pd₂dba₃ (7.02 mg, 7.66 μmol), and Cs₂CO₃ (59.9 mg, 0.18 mmol), under vacuum was added 1,4-dioxane (20 mL), water (5 mL), and the resulting suspension was heated in an oil bath at 80° C., placed under nitrogen, and held at that temperature for 1 hour. The reaction mixture was cooled to room temperature, diluted with ethyl acetate (100 ml) and washed with water. The aqueous phase was extracted with ethyl acetate (2×50 ml), and the combined organics were washed with brine, dried over magnesium sulfate, filtered, and concentrated under reduced pressure. The residue was purified by flash chromatography on silica gel eluting with a gradient of ethyl acetate in hexanes to give the title compound as a cream colored solid (20 mg, 25.4%).

MS (EI) (M+H)⁺ 515 for C₂₅H₂₃N₈O₃S (M−H)⁻ 513 for C₂₅H₂₁N₈O₃S

¹H NMR (DMSO-d6) δ: 9.50 (s, 1H), 9.16 (d, J=2.07 Hz, 1H), 8.75 (d, J=1.88 Hz, 1H), 8.37 (s, 1H), 8.36 (t, J=2.07 Hz, 1H), 8.33 (s, 1H), 8.28 (s, 1H), 7.69 (t, J=7.82 Hz, 1H), 7.60 (t, J=5.75 Hz, 1H), 7.23 (d, J=7.35 Hz, 1H), 6.77 (d, J=8.10 Hz, 1H), 3.90 (s, 3H), 3.17-3.28 (m, 2H), 2.57 (s, 3H), 1.12 (t, J=7.16 Hz, 3H);

### Example 251

### 1-ethyl-3-(4-(4-(6-methoxypyridin-2-yl)thiazol-2-yl)-5-(4-(1-methyl-1H-1,2,4-triazol-5-yl)-5-(5-oxo-4,5-dihydro-1,3,4-oxadiazol-2-yl)thiazol-2-yl)pyridin-2-yl)urea

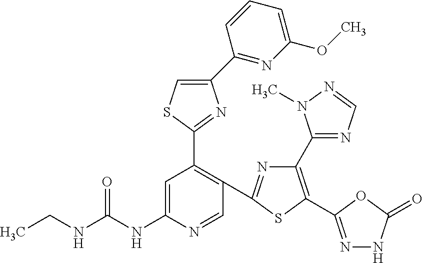

A mixture of 1-ethyl-3-(5-(5-(hydrazinecarbonyl)-4-(1-methyl-1H-1,2,4-triazol-5-yl)thiazol-2-yl)-4-(4-(6-methoxypyridin-2-yl)thiazol-2-yl)pyridin-2-yl)urea (Intermediate 419, 115 mg, 0.20 mmol) and 1,1′-carbonyl diimidazole (120 mg, 0.74 mmol) was suspended in DMF (3 mL) and heated to 110° C. for 20 min. The grey suspension was filtered hot, the filtrate was then treated with water (9 ml) and allowed to cool slowly, the resulting yellow precipitate was collected by filtration, washed with methanol to give the title compound as a pale yellow solid (30 mg, 24.96%).

MS (EI) (M+H)⁺ 604 for C₂₅H₂₂N₁₁O₄S₂ (M−H)⁻ 602 for C₂₅H₂₀N₁₁O₄S₂

¹H NMR (DMSO-d6) δ: 12.58-12.96 (m, 1H), 9.71 (s, 1H), 8.82 (s, 1H), 8.49 (s, 1H), 8.17 (s, 1H), 8.07 (s, 1H), 7.73 (t, J=7.82 Hz, 1H), 7.51 (t, J=5.84 Hz, 1H), 7.41 (d, J=7.35 Hz, 1H), 6.81 (d, J=8.10 Hz, 1H), 3.95 (s, 3H), 3.79 (s, 3H), 3.13-3.28 (m, 2H), 1.11 (t, J=7.16 Hz, 3H);

### Example 252

### 1-ethyl-3-(4-(4-(6-methoxypyridin-2-yl)thiazol-2-yl)-5-(5-(5-methyl-1,3,4-oxadiazol-2-yl)-4-(1-methyl-1H-1,2,4-triazol-5-yl)thiazol-2-yl)pyridin-2-yl)urea

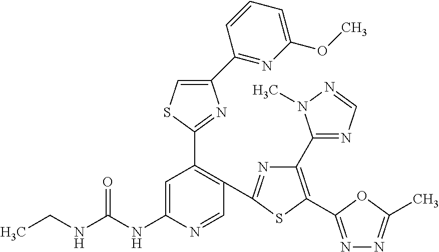

A suspension of 1-ethyl-3-(5-(5-(hydrazinecarbonyl)-4-(1-methyl-1H-1,2,4-triazol-5-yl)thiazol-2-yl)-4-(4-(6-methoxypyridin-2-yl)thiazol-2-yl)pyridin-2-yl)urea (Intermediate 419, 50 mg, 0.08 mmol) in DMF (2 ml) and 1,1,1-trimethoxyethane (5 ml, 41.62 mmol) was treated with aqueous HCl (1 drop), heated to 100° C. for 15 minutes, then treated with DBU (1 ml) and refluxed for 5 minutes. The reaction mixture was then cooled, diluted with water (25 ml) and ethyl acetate (100 ml), and the layers were separated. The organic phase was washed sequentially with saturated sodium hydrogen carbonate, brine, then dried over magnesium sulfate. The solvent was removed under reduced pressure and the residue was purified by chromatography on silica gel eluting with a gradient of methanol in methylene chloride. The appropriate fractions were pooled and the crude product was precipitated from ethyl acetate with hexanes to give product as a pale yellow solid (15 mg, 15%).

MS (EI) (M+H)⁺ 602 for C₂₆H₂₄N₁₁O₃S₂ (M−H)⁻ 600 for C₂₆H₂₂N₁₁O₃S₂

¹H NMR (DMSO-d6) δ: 9.72 (s, 1H), 8.85 (s, 1H), 8.49 (s, 1H), 8.17 (s, 1H), 8.04 (s, 1H), 7.71 (t, J=7.82 Hz, 1H), 7.51 (t, J=4.99 Hz, 1H), 7.41 (d, J=7.35 Hz, 1H), 6.80 (d, J=8.29 Hz, 1H), 3.94 (s, 3H), 3.81 (s, 3H), 3.10-3.28 (m, 2H), 2.50 (br. s., 3H), 1.11 (t, J=7.06 Hz, 3H);

### Example 253

### 1-ethyl-3-(5′-(5-oxo-4,5-dihydro-1,3,4-oxadiazol-2-yl)-4-(4-(pyridin-4-ylmethyl)thiazol-2-yl)-3,3′-bipyridin-6-yl)urea

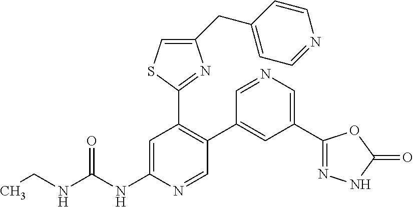

To a mixture of 1-ethyl-3-(5′-(hydrazinecarbonyl)-4-(4-(pyridin-4-ylmethyl)thiazol-2-yl)-3,3′-bipyridin-6-yl)urea (Intermediate 421, 55 mg, 0.12 mmol) in THF (10 ml) was added di(1H-imidazol-1-yl)methanone (75 mg, 0.46 mmol) and the resulting suspension was warmed slightly to afford a solution which was stirred at room temperatere for one hour at which point the solvents were removed under reduced pressure and the resulting solid was dissolved in ethyl acetate (50 ml), methanol (5 ml), and water (50 ml). The layers were separated, and the organic phase was washed sequentially with saturated aqueous sodium hydrogen carbonate and brine, dried over magnesium sulfate, filtered, concentrated, and purified by normal phase chromatography eluting on silica gel with a gradient of methanol in dichloromethane to afford 40 mg (61%) of the title compound as an off white powder.

MS (EI) (M+H)⁺ 501 for C₂₄H₂₁N₈O₃S (M−H)⁻ 499 for C₂₄H₁₉N₈O₃S;

¹H NMR (DMSO-d6) δ: 12.59 (br. s., 1H), 9.47 (s, 1H), 8.89 (d, J=1.70 Hz, 1H), 8.59 (d, J=1.70 Hz, 1H), 8.36 (d, J=5.65 Hz, 2H), 8.29 (s, 1H), 8.06 (s, 1H), 7.93 (s, 1H), 7.67 (br. s., 1H), 7.58 (s, 1H), 7.03 (d, J=5.27 Hz, 2H), 3.99 (s, 2H), 3.20 (dq, J=6.97, 6.59 Hz, 2H), 1.10 (t, J=7.06 Hz, 3H);

### Example 254

### 1-ethyl-3-(6′-(2-methoxyethoxy)-5′-(5-oxo-4,5-dihydro-1,3,4-oxadiazol-2-O-4-(4-(trifluoromethyl)thiazol-2-yl)-3,3′-bipyridin-6-yl)urea

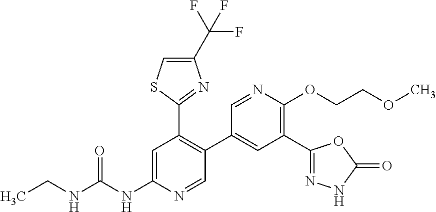

To a solution of ethyl 6′-(3-ethylureido)-6-(2-methoxyethoxy)-4′-(4-(trifluoromethyl)thiazol-2-yl)-3,3′-bipyridine-5-carboxylate (Intermediate 425, 90 mg, 0.05 mmol) in ethanol (10 mL) was added hydrazine (200 μL, 6.24 mmol). The solution was heated to reflux for 1 hour, solvents were removed, and the resulting gum was taken up into THF (10 ml) and treated with 1,1′-carbonyl di-imidazole (2×50 mg). The resulting solution was stirred at RT for 8 hours. The solvents were removed and the residue was purified by normal phase chromatography eluting with a gradient of methanol in dichloromethane, to afford crude product, which was purified by normal phase chromatography eluting with a gradient of ethyl acetate in hexanes to give the title compound as an off white solid.

MS (EI) (M+H)⁺ 551 for C₂₂H₂₁F₃N₇O₅S (M−H)⁻ 549 for C₂₂H₁₉F₃N₇O₅S;

¹H NMR (DMSO-d6) δ: 12.65 (s, 1H), 9.44 (s, 1H), 8.49 (s, 1H), 8.28 (s, 1H), 8.23 (d, J=1.70 Hz, 2H), 7.67 (t, J=4.71 Hz, 1H), 7.15 (s, 1H), 4.49 (dd, J=5.37, 3.11 Hz, 2H), 3.71 (t, J=4.33 Hz, 2H), 3.31 (br. s., 3H), 3.14-3.27 (m, 2H), 1.11 (t, J=7.25 Hz, 3H);

¹⁹F-NMR (DMSO-d6) δ −62.51 (br. s., 3 F);

### Example 255

### 6′-(3-ethylureido)-5-(5-methyl-1,3,4-oxadiazol-2-yl)-4′-(4-(trifluoromethyl)thiazol-2-yl)-3,3′-bipyridine 1-oxide

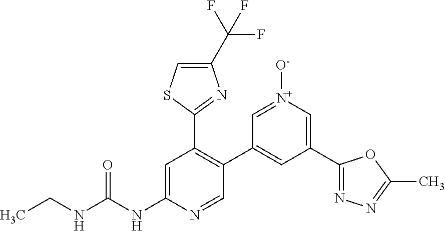

A mixture of 6-(3-ethylureido)-4-(4-(trifluoromethyl)thiazol-2-yl)pyridin-3-ylboronic acid (Intermediate 12, 300 mg, 0.83 mmol), potassium carbonate (142 mg, 1.03 mmol), palladium diphenylphosphinoferocene dichloride (50.0 mg, 0.07 mmol) and 3-bromo-5-(5-methyl-1,3,4-oxadiazol-2-yl)pyridine 1-oxide (Intermediate 428, 175 mg, 0.68 mmol) was treated with acetonitrile (10 mL) under vacuum. Water (10.00 mL) was added and after degassing for 1 minute the suspension was heated to 80° C. for 30 minutes. The reaction was then cooled to RT, diluted with ethyl acetate (100 ml) and methanol (10 ml). The organic layer were washed with water, saturated bicarbonate, and brine, and the aqueous phase was back extracted with ethyl acetate (2×100 ml). The combined organics were washed with brine, dried over magnesium sulfate, filtered, and the solvent removed under reduced pressure. The residue was purified by normal phase chromatography on silica gel eluting with a gradient of methanol in dichloromethane. The major peak was concentrated down to a pale amber solid which was suspended in dichloromethane and filtered to give 170 mg of the title compound as a white solid.

MS (EI) (M+H)⁺ 492 for C₂₀H₁₇F₃N₇O₃S (M−H) 490 for C₂₀H₁₅F₃N₇O₃S;

¹H NMR (DMSO-d6) δ: 9.54 (s, 1H), 8.71 (s, 1H), 8.63 (s, 1H), 8.53 (s, 1H), 8.40 (s, 1H), 8.23 (s, 1H), 7.77 (s, 1H), 7.52 (t, J=4.99 Hz, 1H), 3.14-3.28 (m, J=7.16, 7.16, 6.22, 6.22 Hz, 2H), 2.58 (s, 3H), 1.10 (t, J=7.16 Hz, 3H);

¹⁹F-NMR (DMSO-d6) δ: −62.57 (s, 3 F);

### Example 256

### 1-(5′-(5-(difluoromethyl)-4H-1,2,4-triazol-3-yl)-4-(4-(trifluoromethyl)thiazol-2-yl)-3,3′-bipyridin-6-yl)-3-ethylurea

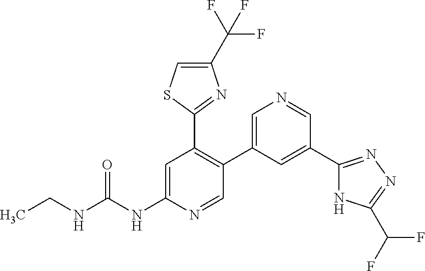

A mixture of 3-bromo-5-(5-(difluoromethyl)-4H-1,2,4-triazol-3-yl)pyridine (Intermediate 429, 76 mg, 0.28 mmol), 6-(3-ethylureido)-4-(4-(trifluoromethyl)thiazol-2-yl)pyridin-3-ylboronic acid (Intermediate 12, 100 mg, 0.28 mmol), cesium carbonate (181 mg, 0.56 mmol), DicyclohexylTriisopropylBiphenylphosphine (39.7 mg, 0.08 mmol), and dipalladium(0)trisdibenzilidineacetone (12.71 mg, 0.01 mmol) in 1,4-dioxane (12 mL) was degassed, treated with water (3 mL) and heated to 80° C. for 30 minutes. The reaction was diluted with ethyl acetate (100 ml) and water (100 ml) and the layers were separated. The aqueous phase was extracted with ethyl acetate (3×50 ml), and the combined organics were washed with brine, dried over magnesium sulfate, and filtered. The solvent was removed under reduced pressure, and the residue was purified by normal phase chromatography on silica gel, first eluting with a gradient of ethyl acetate in hexanes to provide crude product which was further purified by normal phase chromatography on silica gel eluting with a gradient of methanol in dichloromethane to afford a tan solid which was triturated from dichloromethane with hexanes to afford 50 mg of the title compound as a beige solid.

MS (EI) (M+H)⁺ 511 for C₂₀H₁₆F₅N₈OS (M−H)⁻ 509 for C₂₀H₁₄F₅N₈OS;

¹H NMR (DMSO-d6) δ: 15.19 (br. s., 1H), 9.52 (s, 1H), 9.22 (d, J=0.94 Hz, 1H), 8.64 (s, 1H), 8.55 (s, 1H), 8.40 (s, 1H), 8.34 (br. s., 1H), 8.27 (s, 1H), 7.55 (t, J=5.09 Hz, 1H), 7.19 (t, J=53.50 Hz, 1H), 3.21 (quin, J=6.73 Hz, 2H), 1.11 (t, J=7.06 Hz, 3H);

¹⁹F-NMR (DMSO-d₆) δ: −62.49 (s, 3 F), −116.16 (br. s., 2 F);

### Example 257

### 1-ethyl-3-(5′-(5-(trifluoromethyl)-4H-1,2,4-triazol-3-yl)-4-(4-(trifluoromethyl)thiazol-2-yl)-3,3′-bipyridin-6-yl)urea

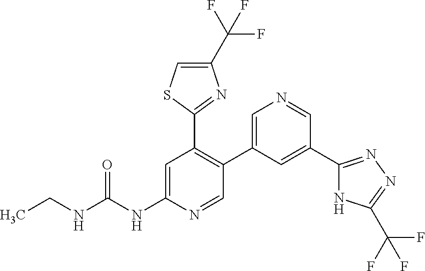

Example 257 was synthesized as described for Example 256 from Intermediate 431 and Intermediate 12 as a tan solid.

MS (EI) (M+H)⁺ 529 for C₂₀H₁₅F₆N₈OS (M−H)⁻ 527 for C₂₀H₁₃F₆N₈OS

¹H NMR (DMSO-d6) δ: 15.56 (br. s., 1H), 9.52 (s, 1H), 9.22 (s, 1H), 8.66 (s, 1H), 8.52-8.61 (m, 1H), 8.41 (s, 1H), 8.36 (s, 1H), 8.26 (s, 1H), 7.54 (t, J=5.09 Hz, 1H), 3.21 (dq, J=6.97, 6.66 Hz, 2H), 1.11 (t, J=7.16 Hz, 3H);

¹⁹F-NMR (DMSO-d6) δ: −62.52 (s, 3 F), −63.75 (br. s., 3 F);

### Example 258

### 1-(6′-amino-5′-(5-methyl-1,3,4-oxadiazol-2-yl)-4-(4-(trifluoromethyl)thiazol-2-yl)-3,3′-bipyridin-6-yl)-3-ethylurea

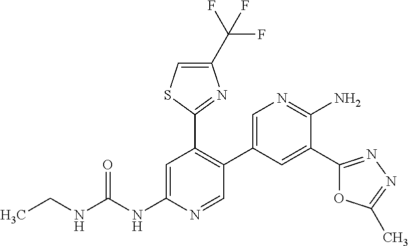

Example 258 was synthesized according to the procedure for Example 255 from Intermediate 12 and Intermediate 434 as an off white solid.

MS (EI) (M+H)⁺ 491 for C₂₀H₁₈F₃N₈O₂S (M−H)⁻ 489 for C₂₀H₁₆F₃N₈O₂S;

¹H NMR (DMSO-d6) δ: 9.41 (s, 1H), 8.52 (s, 1H), 8.30 (s, 1H), 8.25 (s, 1H), 8.13 (d, J=2.26 Hz, 1H), 7.96 (d, J=2.45 Hz, 1H), 7.63 (t, J=4.52 Hz, 1H), 7.53 (br. s., 2H), 3.20 (quin, J=6.59 Hz, 2H), 2.54 (s, 3H), 1.10 (t, J=7.16 Hz, 3H);

¹⁹F-NMR (DMSO-d6) δ: −62.33 (s, 3 F);

### Example 259

### 6-(3-ethylureido)-2′-(5-methyl-1,3,4-oxadiazol-2-yl)-4-(4-(trifluoromethyl)thiazol-2-yl)-3,4′-bipyridine 1′-oxide

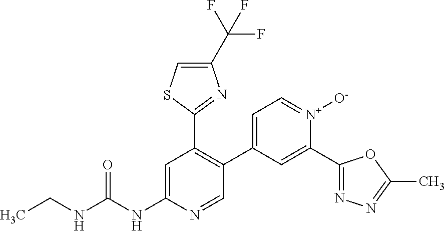

Example 259 was synthesized according to the procedure for Example 255 from Intermediate 12 and Intermediate 436 as a white solid.

MS (EI) (M+H)⁺ 492 for C₂₀H₁₇F₃N₇O₃S (M−H) 490 for C₂₀H¹⁵F₃N₇O₃S;

¹H NMR (DMSO-d6) δ: 9.53 (s, 1H), 8.64 (s, 1H), 8.47 (d, J=6.97 Hz, 1H), 8.40 (s, 1H), 8.18 (s, 1H), 7.91 (d, J=2.45 Hz, 1H), 7.56 (dd, J=6.78, 2.26 Hz, 1H), 7.49 (t, J=4.71 Hz, 1H), 3.11-3.25 (m, 2H), 2.61 (s, 3H), 1.10 (t, J=7.16 Hz, 3H);

¹⁹F-NMR (DMSO-d6) δ: −62.47 (s, 3 F);

### Example 260

### 1-(2′-amino-5′-(5-oxo-4,5-dihydro-1,3,4-oxadiazol-2-yl)-4-(4-(trifluoromethyl)thiazol-2-yl)-3,3′-bipyridin-6-yl)-3-ethylurea

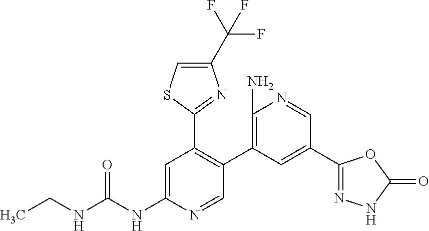

Example 260 was synthesized according to the procedure for Example 255 from Intermediate 12 and Intermediate 437 as a pale yellow solid.

MS (EI) (M+H)⁺ 493 for C₁₉H₁₆F₃N₈O₃S (M−H)⁻ 491 for C₁₉H₁₄F₃N₈O₃S;

¹H NMR (DMSO-d6) δ: 12.40 (br. s., 1H), 9.43 (s, 1H), 8.50 (s, 1H), 8.48 (d, J=2.26 Hz, 1H), 8.34 (s, 1H), 8.17 (s, 1H), 7.82 (t, J=5.56 Hz, 1H), 7.69 (d, J=2.26 Hz, 1H), 6.51 (br. s., 2H), 3.15-3.27 (m, 2H), 1.10 (t, J=7.16 Hz, 3H)

¹⁹F-NMR (DMSO-d₆) δ: −62.29 (s, 3 F)

### Example 261

### 1-ethyl-3-(5-(5-(5-methyl-1,3,4-oxadiazol-2-yl)-6-oxo-1,6-dihydropyridin-3-yl)-4-(4-(trifluoromethyl)thiazol-2-yl)pyridin-2-yl)urea

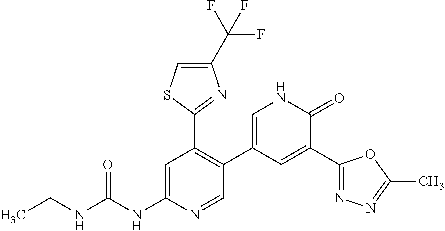

Perchloromethane (19.32 mg, 0.13 mmol) was added to a solution of crude 1454542-acetylhydrazinecarbonyl)-6-oxo-1,6-dihydropyridin-3-yl)-4-(4-(trifluoromethyl)thiazol-2-yl)pyridin-2-yl)-3-ethylurea (Intermediate 441, 16 mg, 0.03 mmol), triphenylphosphine (16.47 mg, 0.06 mmol), and DBU (9.47 μL, 0.06 mmol) in acetonitrile (5 mL) and the mixture was stirred for 80 hours at RT. The reaction was purified by normal phase chromatography on silica gel eluting with a gradient of methanol in dichloromethane to afford 10 mg of the title compound as an off white solid.

MS (EI) (M+H)⁺ 492 for C₂₀H₁₇F₃N₇O₃S (M−H)⁻ 490 for C₂₀H₁₅F₃N₇O₃S;

¹H NMR (DMSO-d6) δ: 12.64 (br. s., 1H), 9.42 (s, 1H), 8.60 (s, 1H), 8.32 (s, 1H), 8.24 (s, 1H), 7.97 (d, J=2.64 Hz, 1H), 7.82 (s, 1H), 7.59 (t, J=5.09 Hz, 1H), 3.20 (dq, J=6.97, 6.59 Hz, 2H), 2.52 (br. s., 3H), 1.10 (t, J=7.16 Hz, 3H)

¹⁹F-NMR (DMSO-d6) δ: −62.39 (s, 3 F)

### Example 262

### 1-ethyl-3-(4-(4-(6-methoxypyridin-2-yl)thiazol-2-yl)-5-(6-(5-oxo-4,5-dihydro-1,3,4-oxadiazol-2-yl)pyrazin-2-yl)pyridin-2-yl)urea

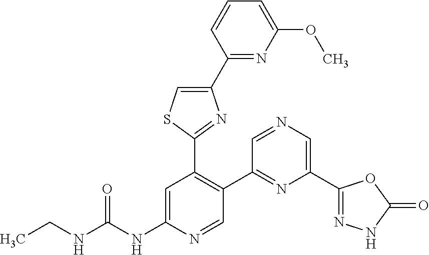

1,1′-Carbonyl di-imidazole (CDI, 50 mg, 0.31 mmol) was added to a solution of crude 1-ethyl-3-(5-(6-(hydrazinecarbonyl)pyrazin-2-yl)-4-(4-(6-methoxypyridin-2-yl)thiazol-2-yl)pyridin-2-yl)urea (Intermediate 446, 30 mg, 0.06 mmol) in tetrahydrofuran (10 mL) and DIEA (100 μL, 0.57 mmol). The amber solution was treated with CDI (3×20 mg), solvent was removed under reduced pressure, purified by normal phase chromatography on silica gel eluting with a gradient of methanol in dichloromethane, the crude product was dissolved in ethyl acetate (50 ml), washed with water (50 ml). The aqueous layer was back extracted with ethyl acetate (2×50 ml), combined organics were washed with brine, dried over magnesium sulfate, filtered, and concentrated to give 23 mg of the title compound as a pale off white solid.

MS (EI) (M+H)⁺ 518 for C₂₃H₂₀N₉O₄S (M−H)⁻ 516 for C₂₃H₁₈N₉O₄S;

¹H NMR (DMSO-d6) δ: 9.64 (s, 1H), 9.06 (s, 1H), 8.77 (s, 1H), 8.49 (s, 1H), 8.36 (s, 1H), 8.28 (s, 1H), 7.71 (d, J=7.91 Hz, 1H), 7.59-7.67 (m, 1H), 6.86-7.10 (m, 2H), 6.76 (d, J=8.29 Hz, 1H), 3.91 (s, 3H), 3.22 (quin, J=6.69 Hz, 2H), 1.12 (t, J=7.25 Hz, 3H)

### Example 263

### 1-ethyl-3-(5′-(5-(2-hydroxypropan-2-yl)-1,3,4-oxadiazol-2-yl)-4-(4-(trifluoromethyl)thiazol-2-yl)-3,3′-bipyridin-6-yl)urea

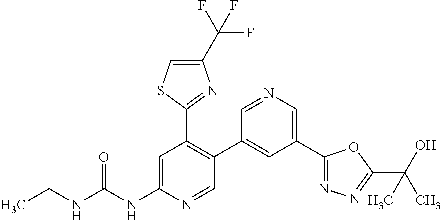

Potassium carbonate (1 ml, 1N in water) was added to a solution of 2-(5-(6′-(3-ethylureido)-4′-(4-(trifluoromethyl)thiazol-2-yl)-3,3′-bipyridin-5-yl)-1,3,4-oxadiazol-2-yl)propan-2-yl acetate (Example 264, 50 mg, 0.09 mmol) in methanol (5 mL) and stirred at RT for 1 hour, at which time solvents were removed and the residue was purified by normal phase chromatography on silica gel eluting with a gradient of methanol in methylene chloride to afford 15 mg of the title compound as a white solid.

MS (EI) (M+H)⁺ 520 for C₂₂H₂₁F₃N₇O₃S (M−H) 518 for C₂₂H₁₉F₃N₇O₃S;

¹H NMR (DMSO-d6) δ: 9.53 (s, 1H), 9.21 (d, J=1.88 Hz, 1H), 8.72 (d, J=1.88 Hz, 1H), 8.57 (s, 1H), 8.42 (s, 1H), 8.33 (t, J=1.98 Hz, 1H), 8.24 (s, 1H), 7.57 (t, J=5.18 Hz, 1H), 5.96 (s, 1H), 3.21 (qd, J=7.16, 6.03 Hz, 2H), 1.60 (s, 6H), 1.11 (t, J=7.16 Hz, 3H);

¹⁹F-NMR (DMSO-d6) δ: −62.57 (s, 3 F);

### Example 264

### 2-(5-(6′-(3-ethylureido)-4′-(4-(trifluoromethyl)thiazol-2-yl)-3,3′-bipyridin-5-yl)-1,3,4-oxadiazol-2-yl)propan-2-yl acetate

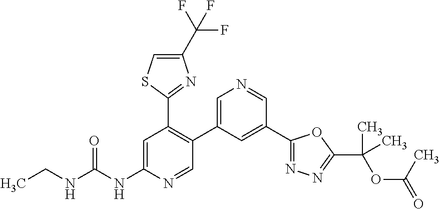

A solution of triphenyl phosphine (55 mg, 0.2 mmol) and DIEA (0.15 ml) in acetonitrile (2 ml) was added to 1-(2-(6′-(3-ethylureido)-4′-(4-(trifluoromethyl)thiazol-2-yl)-3,3′-bipyridine-5-carbonyl)hydrazinyl)-2-methyl-1-oxopropan-2-yl acetate (Intermediate 448, 50 mg, 0.1 mmol). The resulting solution was treated with carbontetrachloride (0.1 ml) and let stir at RT for 2 hours. Volatiles were removed under reduced pressure and the residue was purified by normal phase chromatography on silica gel eluting with a gradient of ethyl acetate in hexanes to afford 30 mg of the title compound as an off white solid.

MS (EI) (M+H)⁺ 562 for C₂₄H₂₃F₃N₇O₄S (M−H)⁻ 560 for C₂₄H₂₁F₃N₇O₄S;

¹H NMR (DMSO-d6) δ: 9.53 (s, 1H), 9.20 (br. s., 1H), 8.73 (br. s., 1H), 8.57 (s, 1H), 8.42 (s, 1H), 8.32 (br. s., 1H), 8.22 (s, 1H), 7.45-7.70 (m, 1H), 3.14-3.28 (m, 2H), 2.04 (s, 3H), 1.79 (s, 6H), 1.11 (t, J=7.06 Hz, 3H);

¹⁹F-NMR (DMSO-d6) δ: −62.61 (s, 3 F);

### Examples 265-279

The following Examples were prepared according to the procedure for Example 264 using the starting materials indicated in the table.

| Ex | Compound | Data | SM |
|---|---|---|---|
| 265 | 1-ethyl-3-(5′-(5-(2-oxopropyl)-1,3,4- oxadiazol-2-yl)-4-(4-(trifluoromethyl)thiazol- 2-yl)-3,3′-bipyridin-6-yl)urea 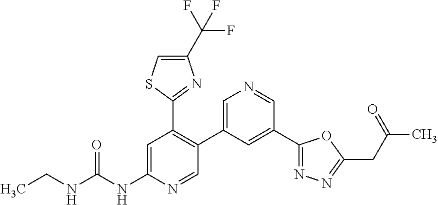 | MS (EI) (M + H)⁺ 518 for C₂₂H₁₉F₃N₇O₅S (M − H)⁻ 516 for C₂₂H₁₇F₃N₇O₅S; ¹H NMR (DMSO-d6) δ: 9.52 (s, 1H), 9.17 (d, J = 2.07 Hz, 1H), 8.72 (d, J = 2.07 Hz, 1H), 8.56 (s, 1H), 8.41 (s, 1H), 8.30 (t, J = 2.07 Hz, 1H), 8.24 (s, 1H), 7.48-7.65 (m, 1H), 4.40 (s, 2H), 3.15-3.25 (m, 2H), 2.28 (s, 3H), 1.11 (t, J = 7.16 Hz, 3H); ¹⁹F-NMR (DMSO-d₆) δ: −62.56 (s, 3F) | Intermediate 449 |
| 266 | 1-(5′-(5-(benzyloxymethyl)-1,3,4- oxadiazol-2-yl)-4-(4-(trifluoromethyl)thiazol- 2-yl)-3,3′-bipyridin-6-yl)-3-ethylurea 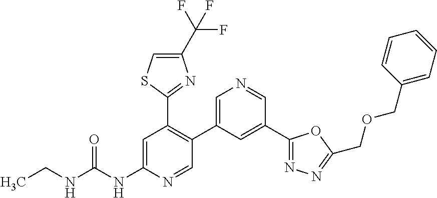 | MS (EI) (M + H)⁺ 582 for C₂₇H₂₃F₃N₇O₃S (M − H)⁻ 580 for C₂₇H₂₁F₃N₇O₃S; ¹H NMR (DMSO-d6) δ: 9.53 (br. s., 1H), 9.19 (br. s., 1H), 8.72 (br. s., 1H), 8.56 (br. s., 1H), 8.40 (br. s., 1H), 8.33 (br. s., 1H), 8.24 (br. s., 1H), 7.59 (br. s., 3H), 7.22-7.43 (m, 3H), 4.85 (br. s., 2H), 4.65 (br. s., 2H), 3.21 (br. s., 2H), 1.11 (br. s., 3H); ¹⁹F-NMR (DMSO- d₆) δ: −62.59 (s, 3F) | Intermediate 450 |
| 267 | 1-(5′-(5-(diethylamino)-1,3,4-oxadiazol-2-yl)-4- (4-(trifluoromethyl)thiazol-2-yl)-3,3′-bipyridin-6-yl)- 3-ethylurea 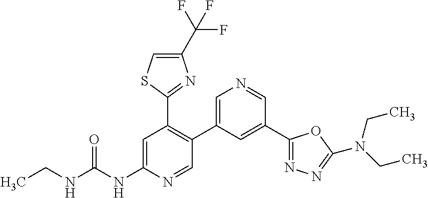 | MS (EI) (M + H)⁺ 533 for C₂₃H₂₄F₃N₈O₂S (M − H)⁻ 531 for C₂₃H₂₂F₃N₈O₂S; ¹H NMR (DMSO-d6) δ: 9.51 (s, 1H), 9.08 (d, J = 2.07 Hz, 1H), 8.62 (d, J = 2.07 Hz, 1H), 8.57 (s, 1H), 8.40 (s, 1H), 8.23 (s, 1H), 8.14 (t, J = 1.98 Hz, 1H), 7.58 (t, J = 5.75 Hz, 1H), 3.48 (q, J = 7.28 Hz, 4H), 3.13-3.27 (m, J = 7.54, 6.97, 6.97, 5.84 Hz, 2H), 1.17 (t, J = 7.06 Hz, 6H), 1.11 (t, J = 7.25 Hz, 3H); ¹⁹F-NMR (DMSO-d₆) δ: −62.52 (s, 3F); | Intermediate 451 |
| 268 | 1-(5′-(5-((dimethylamino)methyl)- 1,3,4-oxadiazol-2-yl)-4-(4-(trifluoromethyl)thiazol- 2-yl)-3,3′-bipyridin-6-yl)-3-ethylurea 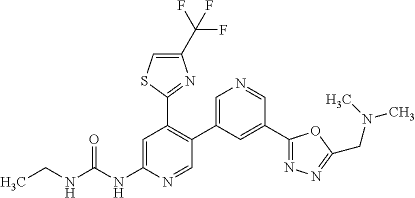 | MS (EI) (M + H)⁺ 519 for C₂₂H₂₂F₃N₈O₂S (M − H)⁻ 517 for C₂₂H₂₀F₃N₈O₂S; ¹H NMR (DMSO-d6) δ: 9.52 (s, 1H), 9.19 (d, J = 1.88 Hz, 1H), 8.73 (d, J = 1.88 Hz, 1H), 8.57 (s, 1H), 8.41 (s, 1H), 8.30 (t, J = 1.98 Hz, 1H), 8.23 (s, 1H), 7.46-7.70 (m, 1H), 3.83 (s, 2H), 3.21 (quin, J = 6.31 Hz, 2H), 2.26 (s, 6H), 1.11 (t, J = 7.16 Hz, 3H); ¹⁹F-NMR (DMSO-d₆) δ: −62.59 (s, 3F); | Intermediate 452 |
| 269 | (9H-fluoren-9-yl)methyl(5-(6′-(3-ethylureido)-4′- (4-(trifluoromethyl)thiazol-2-yl)-3,3′-bipyridin-5-yl)- 1,3,4-oxadiazol-2-yl)methylcarbamate 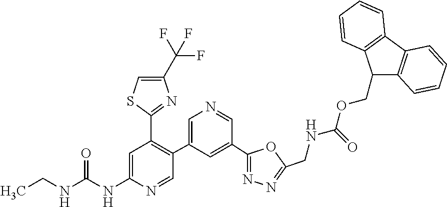 | MS (EI) (M + H)⁺ 713 for C₃₅H₂₈F₃N₈O₄S (M − H)⁻ 711 for C₃₅H₂₆F₃N₈O₄S; ¹H NMR (DMSO-d6) δ: 9.52 (s, 1H), 9.15 (d, J = 2.07 Hz, 1H), 8.71 (d, J = 2.26 Hz, 1H), 8.54 (s, 1H), 8.38 (s, 1H), 8.25-8.29 (m, 1H), 8.23 (s, 1H), 8.13-8.20 (m, 1H), 7.86 (d, J = 7.91 Hz, 2H), 7.69 (d, J = 7.16 Hz, 2H), 7.56 (br. s., 1H), 7.33-7.42 (m, 2H), 7.24-7.33 (m, 2H), 4.54 (d, J = 5.46 Hz, 2H), 4.37 (d, J = 6.97 Hz, 2H), 4.15-4.29 (m, 1H), 3.13-3.27 (m, 2H), 1.11 (t, J = 7.16 Hz, 3H); ¹⁹F-NMR (DMSO-d₆) δ: −62.56 (s, 3F); | Intermediate 453 |
| 270 | 1-ethyl-3-(5′-(5-(methoxymethyl)-1,3,4- oxadiazol-2-yl)-4-(4-(trifluoromethyl)thiazol- 2-yl)-3,3′-bipyridin-6-yl)urea 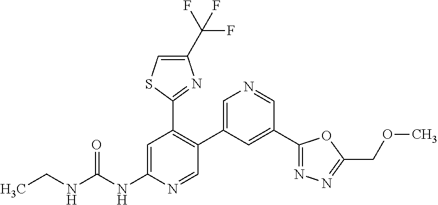 | MS (EI) (M + H)⁺ 506 for C₂₁H₁₉F₃N₇O₃S (M − H)⁻ 504 for C₂₁H₁₇F₃N₇O₃S; ¹H NMR (DMSO-d6) δ: 9.53 (s, 1H), 9.20 (d, J = 1.70 Hz, 1H), 8.73 (d, J = 1.88 Hz, 1H), 8.57 (s, 1H), 8.41 (s, 1H), 8.34 (t, J = 1.88 Hz, 1H), 8.23 (s, 1H), 7.56 (br. s., 1H), 4.74 (s, 2H), 3.35 (s, 3H), 3.15-3.28 (m, 2H), 1.11 (t, J = 7.16 Hz, 3H); ¹⁹F- NMR (DMSO-d₆) δ: −62.59 (s, 3F); | Intermediate 454 |
| 271 | 1-(5′-(5-ethoxy-1,3,4-oxadiazol-2-yl)-4-(4- (trifluoromethyl)thiazol-2-yl)-3,3′-bipyridin-6-yl)- 3-ethylurea 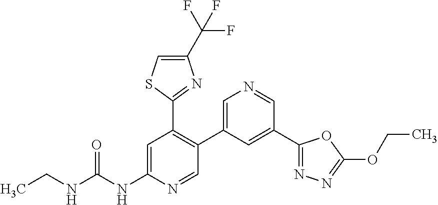 | MS (EI) (M + H)⁺ 506 for C₂₁H₁₉F₃N₇O₃S (M − H)⁻ 504 for C₂₁H₁₇F₃N₇O₃S; ¹H NMR (DMSO-d6) δ: 9.52 (s, 1H), 9.08 (d, J = 2.07 Hz, 1H), 8.68 (d, J = 1.88 Hz, 1H), 8.58 (s, 1H), 8.39 (s, 1H), 8.23 (s, 1H), 8.21 (t, J = 2.07 Hz, 1H), 7.60-7.67 (m, 1H), 4.58 (q, J = 6.97 Hz, 2H), 3.14-3.27 (m, 2H), 1.42 (t, J = 7.06 Hz, 3H), 1.11 (t, J = 7.16 Hz, 3H); ¹⁹F-NMR (DMSO-d₆) δ: −62.53 (s, 3F) | Intermediate 455 |
| 272 | 1-(5′-(1,3,4-oxadiazol-2-yl)-4-(4- (trifluoromethyl)thiazol-2-yl)-3,3′-bipyridin-6-yl)- 3-ethylurea 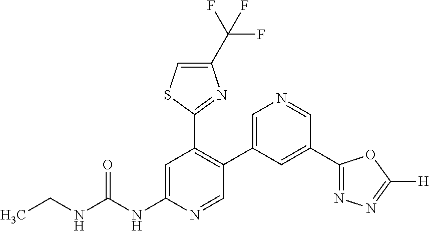 | MS (EI) (M + H)⁺ 462 for C₁₉H₁₅F₃N₇O₂S (M − H)⁻ 460 for C₁₉H₁₃F₃N₇O₂S; ¹H NMR (MEO-D) δ: 9.25 (d, J = 2.07 Hz, 1H), 9.10 (s, 1H), 8.67 (d, J = 2.07 Hz, 1H), 8.43 (t, J = 2.07 Hz, 1H), 8.42 (s, 1H), 8.26 (s, 1H), 7.88 (s, 1H), 3.32-3.41 (m, 2H), 1.23 (t, J = 7.25 Hz, 3H); ¹⁹F-NMR (MEOD) δ: −65.63 (s, 3F); | Intermediate 456 |
| 273 | 1-ethyl-3-(5′-(5-(1-hydroxycyclopropyl)- 1,3,4-oxadiazol-2-yl)-4-(4-(trifluoromethyl) thiazol-2-yl)-3,3′-bipyridin-6-yl)urea 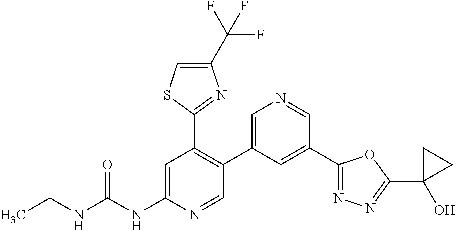 | MS (EI) (M + H)⁺ 518 for C₂₂H₁₉F₃N₇O₃S (M − H)⁻ 516 for C₂₂H₁₇F₃N₇O₃S; ¹H NMR (CHLOROFORM-d) δ: 9.16 (d, J = 1.70 Hz, 1H), 8.75 (br. s., 1H), 8.53 (d, J = 1.88 Hz, 1H), 8.25 (t, J = 1.98 Hz, 1H), 8.21 (s, 1H), 7.78 (s, 1H), 7.65 (s, 1H), 3.65 (s, 1H), 3.15-3.44 (m, 2H), 1.37 (d, J = 2.45 Hz, 4H), 1.20 (t, J = 7.25 Hz, 3H); ¹⁹F-NMR (CHLOROFORM-d) δ: −64.30 (s, 3F); | Intermediate 457 |
| 274 | 5-(6′-(3-ethylureido)-4′-(4-(trifluoromethyl) thiazol-2-yl)-3,3′-bipyridin-5-yl)-N,N-dimethyl-1,3,4- oxadiazole-2-carboxamide 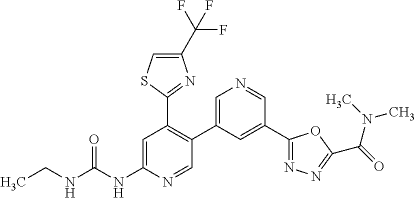 | MS (EI) (M + H)⁺ 533 for C₂₂H₂₀F₃N₈O₃S (M − H)⁻ 531 for C₂₂H₁₈F₃N₈O₃S; ¹H NMR (DMSO-d6) δ: 9.53 (s, 1H), 9.24 (d, J = 2.26 Hz, 1H), 8.76 (d, J = 2.07 Hz, 1H), 8.59 (s, 1H), 8.42 (s, 1H), 8.40 (t, J = 2.07 Hz, 1H), 8.25 (s, 1H), 7.48-7.62 (m, 0H), 3.33 (s, 3H), 3.22 (dd, J = 7.72, 6.03 Hz, 2H), 3.08 (s, 3H), 1.11 (t, J = 7.16 Hz, 3H); ¹⁹F-NMR (DMSO-d₆) δ: −62.54 (s, 3F); | Intermediate 458 |
| 275 | 1-ethyl-3-(5′-(5-(1-hydroxyethyl)-1,3,4- oxadiazol-2-yl)-4-(4-(trifluoromethyl)thiazol- 2-yl)-3,3′-bipyridin-6-yl)urea 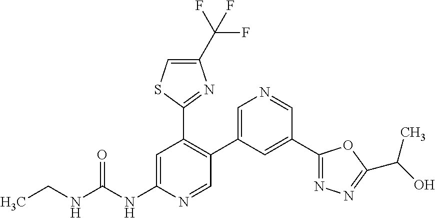 | MS (EI) (M + H)⁺ 506 for C₂₁H₁₉F₃N₇O₃S (M − H)⁻ 504 for C₂₁H₁₇F₃N₇O₃S; ¹H NMR (DMSO-d6) δ: 9.53 (s, 1H), 9.20 (d, J = 2.07 Hz, 1H), 8.72 (d, J = 2.07 Hz, 1H), 8.57 (s, 1H), 8.42 (s, 1H), 8.33 (t, J = 2.07 Hz, 1H), 8.25 (s, 1H), 7.57 (t, J = 5.09 Hz, 1H), 6.07 (d, J = 5.65 Hz, 1H), 5.02 (dq, J = 6.59, 6.28 Hz, 1H), 3.18-3.27 (m, 2H), 1.53 (d, J = 6.59 Hz, 3H), 1.11 (t, J = 7.16 Hz, 3H); ¹⁹F-NMR (DMSO- d₆) δ: −62.56 (s, 3F); | Intermediate 459 |
| 276 | (5-(6′-(3-ethylureido)-4′-(4-(trifluoromethyl)thiazol- 2-yl)-3,3′-bipyridin-5-yl)-1,3,4-oxadiazol-2- yl)methyl acetate 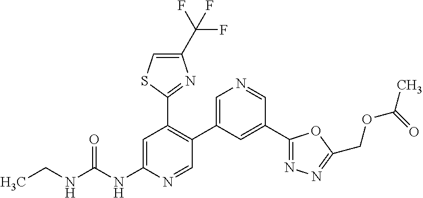 | MS (EI) (M + H)⁺ 534 for C₂₂H₁₉F₃N₇O₄S (M − H)⁻ 532 for C₂₂H₁₇F₃N₇O₄S; ¹H NMR (DMSO-d6) δ: 9.52 (br. s., 1H), 9.19 (br. s., 1H), 8.73 (br. s., 1H), 8.57 (br. s., 1H), 8.41 (br. s., 1H), 8.33 (br. s., 1H), 8.23 (br. s., 1H), 7.57 (br. s., 1H), 5.40 (br. s., 2H), 3.21 (br. s., 2H), 2.13 (br. s., 3H), 1.11 (br. s., 3H); ¹⁹F-NMR (DMSO-d₆) δ: −62.60 (s, 3F) | Intermediate 460 |
| 277 | (S)-tert-butyl 1-(5-(6′-(3-ethylureido)-4′-(4- (trifluoromethyl)thiazol-2-yl)-3,3′-bipyridin-5-yl)- 1,3,4-oxadiazol-2-yl)-2-methylpropylcarbamate 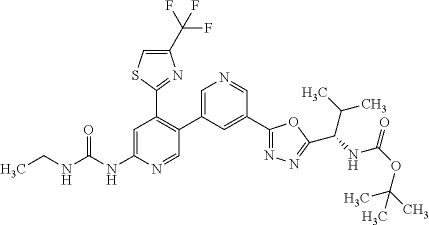 | MS (EI) (M + H)⁺ 633 for C₂₈H₃₂F₃N₈O₄S; ¹H NMR (DMSO-d6) δ: 9.53 (s, 1H), 9.18 (d, J = 1.88 Hz, 1H), 8.73 (d, J = 1.88 Hz, 1H), 8.57 (s, 1H), 8.42 (s, 1H), 8.27 (s, 1H), 8.24 (s, 1H), 7.42-7.77 (m, 2H), 4.68 (t, J = 7.82 Hz, 1H), 3.12-3.27 (m, J = 7.16, 7.16, 7.16, 6.03 Hz, 2H), 2.08- 2.24 (m, 1H), 1.38 (s, 9H), 1.11 (t, J = 7.16 Hz, 3H), 0.92-1.01 (m, 3H), 0.85 (dd, J = 7.06, 2.92 Hz, 3H); NMR (DMSO-d₆) δ: −62.62 (s, 3F); | Intermediate 461 |
| 278 | 1-ethyl-3-(5′-(5-((2-methoxyethoxy)methyl)- 1,3,4-oxadiazol-2-yl)-4-(4-(trifluoromethyl)thiazol- 2-yl)-3,3′-bipyridin-6-yl)urea 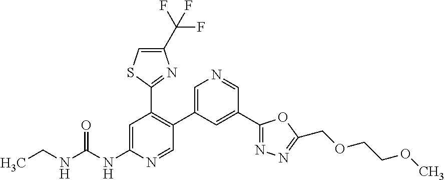 | MS (EI) (M + H)⁺ 550 for C₂₃H₂₃F₃N₇O₄S (M − H)⁻ 548 for C₂₃H₂₁F₃N₇O₄S; ¹H NMR (DMSO-d6) δ: 9.52 (s, 1H), 9.21 (d, J = 1.88 Hz, 1H), 8.73 (d, J = 2.07 Hz, 1H), 8.57 (s, 1H), 8.41 (s, 1H), 8.34 (t, J = 2.07 Hz, 1H), 8.24 (s, 1H), 7.56 (br. s., 1H), 4.82 (s, 2H), 3.69 (dd, J = 5.46, 3.58 Hz, 2H), 3.48 (dd, J = 5.37, 3.67 Hz, 2H), 3.15-3.27 (m, 5H), 1.11 (t, J = 7.16 Hz, 3H); ¹⁹F-NMR (DMSO-d₆) δ: −62.57 (s, 3F) | Intermediate 462 |
| 279 | 1-(5′-(5-(1-aminocyclopropyl)-1,3,4- oxadiazol-2-yl)-4-(5-methyl-4-(trifluoromethyl) thiazol-2-yl)-3,3′-bipyridin-6-yl)-3-ethylurea 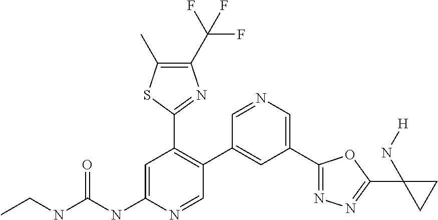 | MS (EI) (M + H)⁺ 531 for C₂₃H₂₂F₃N₈O₂S (M − H)⁻ 529 for C₂₃H₂₂F₃N₈O₂S; ¹H NMR (DMSO-d6) δ: 9.49 (s, 1H), 9.19 (d, J = 2.07 Hz, 1H), 8.68 (d, J = 2.26 Hz, 1H), 8.36 (s, 1H), 8.33 (t, J = 2.17 Hz, 1H), 8.19 (s, 1H), 7.56 (t, J = 5.27 Hz, 1H), 3.10- 3.27 (m, 2H), 2.66-2.84 (m, 2H), 2.51-2.55 (m, 3H), 1.26-1.35 (m, 2H), 1.06-1.16 (m, 5H); ¹⁹F-NMR (DMSO-d₆) δ: −59.69 (s, 3F) | Intermediate 463 |

### Example 280

### (R)-1-(5′-(5-(1-amino-2-methylpropyl)-1,3,4-oxadiazol-2-yl)-4-(4-(trifluoromethyl)thiazol-2-yl)-3,3′-bipyridin-6-yl)-3-ethylurea

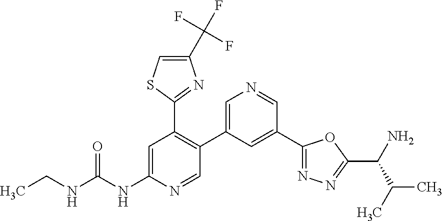

Hydrochloric acid (3 ml, 1M in 1,4-dioxane) was added to a solution of (R)-tert-butyl 145-(6′-(3-ethylureido)-4′-(4-(trifluoromethyl)thiazol-2-yl)-3,3′-bipyridin-5-yl)-1,3,4-oxadiazol-2-yl)-2-methylpropylcarbamate (Intermediate 468, 75 mg, 0.12 mmol) in 1,4-dioxane (10 mL) and methanol (5 ml), warmed to 50° C. for 1 hour, solvent was removed and the residue was precipitated from methanol with ethyl acetate to afford 50 mg of the HCl salt of the title compound as a white solid.

MS (EI) (M+H)⁺ 533 for C₂₃H₂₆F₃N₈O₃S (M−H)⁻ 531 for C₂₃H₂₄F₃N₈O₃S;

¹H NMR (DMSO-d6) δ: 9.57 (s, 1H), 9.23 (d, J=2.07 Hz, 1H), 9.00 (d, J=1.51 Hz, 3H), 8.77 (d, J=2.07 Hz, 0H), 8.60 (s, 0H), 8.42 (s, 0H), 8.34 (t, J=1.88 Hz, 0H), 8.26 (s, 1H), 7.52-7.61 (m, 0H), 4.74 (d, J=4.52 Hz, 1H), 3.17-3.27 (m, 2H), 2.33-2.43 (m, 1H), 1.11 (1, J=7.25 Hz, 3H), 1.06 (d, J=6.78 Hz, 3H), 0.94 (d, J=6.78 Hz, 3H);

¹⁹F-NMR (DMSO-d6) δ: −62.60 (s, 3 F);

### Example 281

### 1-(5′-(5-(aminomethyl)-1,3,4-oxadiazol-2-yl)-4-(4-(trifluoromethyl)thiazol-2-yl)-3,3′-bipyridin-6-yl)-3-ethylurea

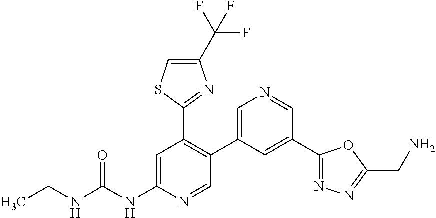

A solution of (9H-fluoren-9-yl)methyl (5-(6′-(3-ethylureido)-4′-(4-(trifluoromethyl)thiazol-2-yl)-3,3′-bipyridin-5-yl)-1,3,4-oxadiazol-2-yl)methylcarbamate (Example 269, 40 mg, 0.06 mmol) in 1,4-dioxane (10 mL) was treated with piperidine (2 mL, 0.06 mmol), and stirred for 1 hour at room temperature, solvents were removed under reduced pressure. The residue was dissolved in methanol (5 ml) and treated with HCl (4 M in dioxane, 0.4 ml), the solution was diluted with ethyl acetate and the resulting solid was isolated by filtration to afford the HCl salt of the title compound as a white solid.

MS (EI) (M+H)⁺ 491 for C₂₀H₁₈F₃N₈O₂S (M−H)⁻ 489 for C₂₀H₁₆F₃N₈O₂S;

¹H NMR (DMSO-d6) δ: 9.66 (br. s., 1H), 9.22 (s, 1H), 8.97 (br. s., 3H), 8.75 (d, J=1.88 Hz, 1H), 8.60 (s, 1H), 8.41 (s, 1H), 8.38 (d, J=1.70 Hz, 1H), 8.29 (s, 1H), 7.63 (br. s., 1H), 4.51 (d, J=5.09 Hz, 2H), 3.17-3.29 (m, 2H), 1.17 (t, J=6.97 Hz, 3H);

¹⁹F-NMR (DMSO-d6) δ: −62.56 (s, 3 F)

### Example 282

### 1-ethyl-3-(5′-(5-oxo-5,6-dihydro-4H-1,3,4-oxadiazin-2-yl)-4-(4-(trifluoromethyl)thiazol-2-yl)-3,3′-bipyridin-6-yl)urea

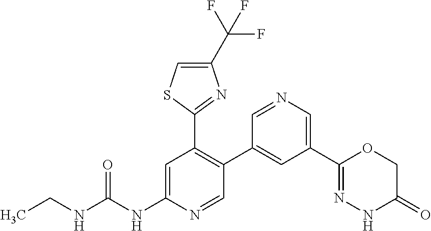

Potassium carbonate (200 mg, 1.45 mmol) was added to a solution of 1-(5′-(2-(2-chloroacetyl)hydrazinecarbonyl)-4-(4-(trifluoromethyl)thiazol-2-yl)-3,3′-bipyridin-6-yl)-3-ethylurea (Intermediate 464, 130 mg, 0.25 mmol) in DMF (3 ml) and the resulting solution was heated to 60° C. for 210 minutes. The reaction was then diluted with ethyl acetate (50 ml), water (20 ml), and saturated ammonium chloride (40 ml). The layers separated and the aqueous phase extracted with ethyl acetate (2×50 ml). The combined organics were washed with brine, dried over magnesium sulfate, filtered, and the solvents removed under reduced pressure. The resulting residue was purified by normal phase chromatography on silica gel eluting with a gradient of methanol in methylene chloride to give 26 mg of the title compound as a white powder.

MS (EI) (M+H)⁺ 492 for C₂₀H₁₇F₃N₇O₃S (M−H) 490 for C₂₀H₁₅F₃N₇O₃S;

¹H NMR (DMSO-d6) δ: 11.19 (s, 1H), 9.49 (s, 1H), 8.95 (br. s., 1H), 8.56 (br. s., 2H), 8.34 (s, 1H), 8.22 (s, 1H), 8.03 (br. s., 1H), 7.57 (br. s., 1H), 4.80 (s, 2H), 3.12-3.27 (m, 2H), 1.10 (t, J=6.97 Hz, 3H);

¹⁹F-NMR (DMSO-d6) δ: −62.45 (s, 3 F)

### Example 283

### 1-(5′-(4-amino-5-methyl-4H-1,2,4-triazol-3-yl)-4-(4-(trifluoromethyl)thiazol-2-yl)-3,3′-bipyridin-6-yl)-3-ethylurea

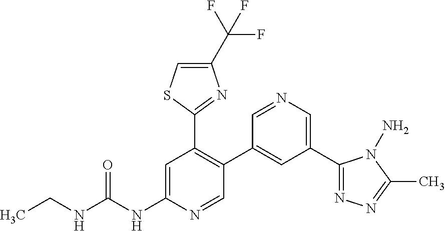

A solution of 1-ethyl-3-(5′-(hydrazinecarbonyl)-4-(4-(trifluoromethyl)thiazol-2-yl)-3,3′-bipyridin-6-yl)urea (Intermediate 9, 250 mg, 0.55 mmol), 1,1-dimethoxy-N,N-dimethylethanamine (0.5 mL, 0.55 mmol) in methanol (5 mL), was stirred at RT for 17 hours, hydrazine (0.2 mL, 0.55 mmol) was added and the suspension was stirred at RT for 10 hours, a small amount of insoluble material was removed by filtration. The filtrate was concentrated under reduced pressure and the residue was purified by normal phase chromatography on silica gel eluting with a gradient of methanol in dichloromethane to give 60 mg of the title compound as a tan solid.

MS (EI) (M+H)⁺ 490 for C₂₀H₁₉F₃N₉OS (M−H)⁻ 488 for C₂₀H₁₇F₃N₉OS;

¹H NMR (DMSO-d6) δ: 9.51 (s, 1H), 9.23 (d, J=1.88 Hz, 1H), 8.59 (d, J=2.07 Hz, 1H), 8.56 (s, 1H), 8.34-8.41 (m, 2H), 8.26 (s, 1H), 7.60 (t, J=5.18 Hz, 1H), 6.06 (s, 2H), 3.14-3.27 (m, 2H), 2.38 (s, 3H), 1.11 (t, J=7.16 Hz, 3H);

¹⁹F-NMR (DMSO-d6) δ: −62.35 (s, 3 F)

### Example 284

### 1-Ethyl-3-(4-(4-phenylthiazol-2-yl)-5-(pyrimidin-5-yl)pyridin-2-yl)urea

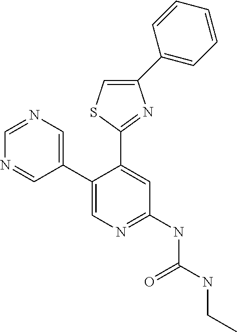

To a nitrogen-purged mixture of 1-(5-bromo-4-(4-phenylthiazol-2-yl)pyridin-2-yl)-3-ethylurea (Intermediate 16, 125 mg, 0.31 mmol) in DME (3 mL) were added pyrimidin-5-ylboronic acid (46.1 mg, 0.37 mmol), sodium bicarbonate (52.1 mg, 0.62 mmol) and water (1 mL), followed by tetrakis(triphenylphosphine)palladium(0) (71.6 mg, 0.06 mmol). The mixture was microwaved for 60 min at 110° C. The solvent was evaporated from the reaction mixture. The crude mass was washed with ethyl acetate and purified on reverse phase preparative HPLC to yield pure 1-ethyl-3-(4-(4-phenylthiazol-2-yl)-5-(pyrimidin-5-yl)pyridin-2-yl)urea (28.0 mg, 22.45%) as white solid powder.

MS (ES⁺): 402.9 for C₂₁H₁₈N₆OS

¹H NMR δ(DMSO D6): 1.1 (t, 3H), 3.2 (qn, 2H), 7.29-7.43 (m, 3H), 7.58 (t, 1H), 7.70 (d, 2H), 8.23 (s, 2H), 8.35 (s, 1H), 8.80 (s, 2H), 9.20 (s, 1H), 9.48 (s, 1H)

### Example 285

### 1-Ethyl-3-(5-(2-methoxypyrimidin-5-yl)-4-(4-phenylthiazol-2-yl)pyridin-2-yl)urea

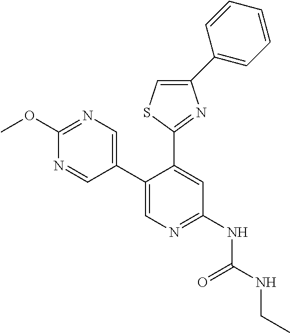

To a nitrogen-purged mixture of 1-(5-bromo-4-(4-phenylthiazol-2-yl)pyridin-2-yl)-3-ethylurea (Intermediate 16, 125 mg, 0.31 mmol) in DME (3 mL) were added 2-methoxypyrimidin-5-ylboronic acid (57.3 mg, 0.37 mmol), sodium bicarbonate (52.1 mg, 0.62 mmol) and water (1 mL), followed by tetrakis(triphenylphosphine)palladium(0) (71.6 mg, 0.06 mmol). The resulting mixture was microwaved for 60 min at 110° C. The solvent was evaporated from the reaction mixture, and the crude mass was washed with ethyl acetate and purified on reverse phase preparative HPLC to yield pure 1-ethyl-3-(5-(2-methoxypyrimidin-5-yl)-4-(4-phenylthiazol-2-yl)pyridin-2-yl)urea (40.0 mg, 29.8%) as white solid powder.

MS (ES⁺): 432.8 for C₂₂H₂₀N₆O₂S

¹H NMR δ(DMSO D6): 1.1 (t, 3H), 3.2 (qn, 2H), 4.0 (s, 3H), 7.32-7.45 (m, 3H), 7.61 (t, 1H), 7.78 (d, 2H), 8.24 (s, 1H), 8.27 (s, 1H), 8.30 (s, 1H), 8.61 (s, 2H), 9.40 (s, 1H)

### Example 286

### 1-Ethyl-3-(6′-fluoro-4-(4-phenylthiazol-2-yl)-3,3′-bipyridin-6-yl)urea

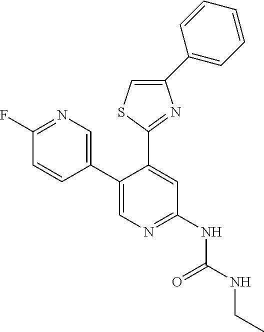

To a nitrogen-purged mixture of 1-(5-bromo-4-(4-phenylthiazol-2-yl)pyridin-2-yl)-3-ethylurea (Intermediate 16, 125 mg, 0.31 mmol) in DME (3 mL) were added 6-fluoropyridin-3-ylboronic acid (65.5 mg, 0.46 mmol), sodium bicarbonate (52.1 mg, 0.62 mmol) and water (1 mL), followed by tetrakis(triphenylphosphine)palladium(0) (71.6 mg, 0.06 mmol). The resulting mixture was microwaved for 60 min at 110° C. When LCMS indicated that the required product had formed and absence of starting material, the solvent was evaporated from the reaction mixture. The crude mass was washed with ethyl acetate and purified on reverse phase preparative HPLC to yield pure 1-ethyl-3-(6′-fluoro-4-(4-phenylthiazol-2-yl)-3,3′-bipyridin-6-yl)urea (45.0 mg, 34.6%).

MS (ES⁺): 419.8 for C₂₂H₁₈FN₅OS

¹H NMR δ(DMSO D6): 1.1 (t, 3H), 3.2 (qn, 2H), 7.24-7.28 (m, 1H), 7.33-7.45 (m, 3H), 7.64 (t, 1H), 7.77-7.80 (m, 2H), 7.93-8.0 (m, 1H), 8.22-8.25 (m, 2H), 8.27 (d, 2H), 9.42 (b, 1H)

### Example 287

### 1-ethyl-3-(4-(1-(2-morpholinoethyl)-1H-pyrazol-4-yl)-5′-(5-oxo-4,5-dihydro-1,3,4-oxadiazol-2-yl)-3,3′-bipyridin-6-yl)urea

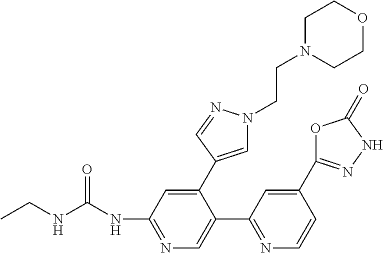

A mixture of (1-(4-bromo-5′-(5-oxo-4,5-dihydro-1,3,4-oxadiazol-2-yl)-3,3′-bipyridin-6-yl)-3-ethylurea (Intermediate 470, 54 mg, 0.13 mmol), 4-(2-(4-(4,4,5,5-tetramethyl-1,3,2-dioxaborolan-2-yl)-1H-pyrazol-1-yl)ethyl)morpholine (43.0 mg, 0.14 mmol), K₂CO₃ (27.6 mg, 0.20 mmol) and tetrkis(triphenylphosphine)palladium (0) (15.40 mg, 0.01 mmol) was suspended in DMF (3.5 ml)/water (0.1 ml) in a microwave reaction vassel, purged with N₂ and heated under microwave at 95° C. for 2 hours. The crude sample was filtered through celite and the filtrate was concentrated and purified by column chromatography on silica gel, eluted with 10% methanol in dichloromethane to give the desired product (25 mg).

MS (ESP) 428.2 (MH⁺) for C₂₄H₂₇N₉O₄.

¹H-NMR (DMSO-d₆): 1.10 (t, 3H); 2.32 (m, 2H); 2.38 (m, 2H); 2.59 (m, 1H); 2.68 (t, 1H); 3.21 (t, 1H); 3.45-3.55 (m, 4H); 4.13-4.24 (m, 3H); 7.03 (s, 1H); 7.18 (s, 1H); 7.63 (t, 1H); 7.72 (t, 1H); 7.97 (s, 1H); 8.17 (s, 1H); 8.58 (d, 1H); 8.95 (s, 1H); 9.31 (s, 1H); 12.80 (br, 1H).

### Example 288

### 1-ethyl-3-(4-(1-(2-morpholinoethyl)-1H-pyrazol-4-yl)-5′-(5-oxo-4,5-dihydro-1,3,4-oxadiazol-2-yl)-3,3′-bipyridin-6-yl)urea

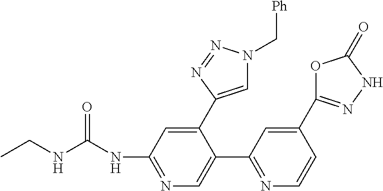

1-Ethyl-3-(4-ethynyl-5′-(5-oxo-4,5-dihydro-1,3,4-oxadiazol-2-yl)-3,3′-bipyridin-6-yl)urea (Intermediate 475, 45 mg, 0.13 mmol), 2,6-lutidine (0.022 ml, 0.19 mmol), copper (I) iodide (2.446 mg, 0.01 mmol) and (azidomethyl)benzene (18.19 mg, 0.13 mmol) were mixed in acetonitrile (10 ml) and NMP (1 ml) and stirred at 65° C. overnight. The reaction mixture was diluted with DCM and filtered through membrane. The filtrate was concentrated and purified by ISCO column (silica gel) eluted with MeOH/DCM (10:1), and then purified again with Gilson (C-18 column, 10%˜85% MeCN in H2O, 0.1% TFA) to give the desired product as a white solid (10 mg).

MS (ESP) 484 (MH⁺) for C₂₄H₂₁N₉O₃

¹H-NMR (DMSO-d₆): 1.10 (t, 3H); 3.20 (m, 2H); 5.51 (s, 2H); 7.15 (m, 2H); 7.27 (m, 3H); 7.72 (m, 1H); 7.87 (m, 2H); 7.98 (s, 1H); 8.24 (s, 1H); 8.62 (d, 1H); 8.94 (d, 1H); 9.42 (s, 1H); 12.82 (br, 1H).

### Intermediate 1

### 6′-{[(Ethylamino)carbonyl]amino}-4′-[4-(trifluoromethyl)-1,3-thiazol-2-yl]-3,3′-bipyridine-5-carboxylic acid

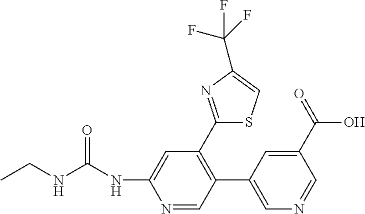

2N LiOH (1 mL) was added to a mixture of ethyl 6′-{[(ethylamino)carbonyl]amino}-4′-[4-(trifluoromethyl)-1,3-thiazol-2-yl]-3,3′-bipyridine-5-carboxylate (Intermediate 2, 0.385 g, 0.83 mmol) in MeOH (3 mL) and THF (3 mL). The resulting solution was stirred at room temperature for two hours. The solvent was removed and the residue was diluted with water and acidified with 1N HCl. The precipitated product was collected by filtration and washed with water and dried (0.297 g).

MS (ES) MH⁺: 437 for C₁₈H₁₄F₃N₅O₃S;

¹H-NMR (DMSO-d₆) δ: 1.11 (t, 3H); 3.18-3.24 (m, 2H); 7.57 (brs, 1H); 8.15-8.18 (m, 1H); 8.22 (s, 1H); 8.37 (s, 1H); 8.57 (s, 1H); 8.72 (s, 1H); 9.08 (s, 1H); 9.51 (s, 1H); 13.53 (s, 1H)

### Intermediate 2

### Ethyl 6′-{[(ethylamino)carbonyl]amino}-4′-[4-(trifluoromethyl)-1,3-thiazol-2-yl]-3,3′-bipyridine-5-carboxylate

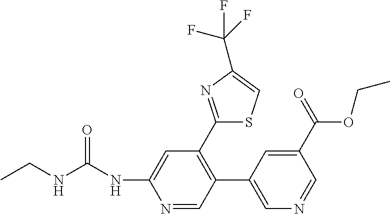

1-(5-Bromo-4-(4-(trifluoromethyl)thiazol-2-yl)pyridin-2-yl)-3-ethylurea (Intermediate 3, 500 mg, 1.27 mmol), cesium carbonate (618 mg, 1.90 mmol), tetrakis(triphenylphosphine)palladium(0) (146 mg, 0.13 mmol), ethyl 5-(4,4,5,5-tetramethyl-1,3,2-dioxaborolan-2-yl)nicotinate (526 mg, 1.52 mmol) were taken in a microwave vial and degassed with Argon. Then dioxane:water (4:1, 8 mL) was added to it and microwaved at 100° C. for half an hour. The reaction mixture was partitioned between water and ethyl acetate and layers separated. The organic layer was washed with saturated sodium bicarbonate solution, water, brine and dried over magnesium sulfate. The solvent was removed and the residue was purified by flash chromatography eluting with 2% MeOH in dichloromethane to 3% MeOH in dichloromethane to give 330 mg of the title compound.

MS (ES) MH⁺: 466 for C₂₀H₁₈F₃N₅O₃S;

¹H-NMR (DMSO-d₆) δ: 1.11 (t, 3H); 1.31 (t, 3H); 3.18-3.24 (m, 2H); 4.34 (q, 2H); 7.57 (brs, 1H); 8.16-8.18 (m, 1H); 8.21 (s, 1H); 8.39 (s, 1H); 8.58 (s, 1H); 8.75 (d, 1H); 9.10 (s, 1H); 9.52 (s, 1H).

### Intermediate 3

### N-{5-Bromo-4-[4-(trifluoromethyl)-1,3-thiazol-2-yl]pyridin-2-yl}-N′-ethylurea

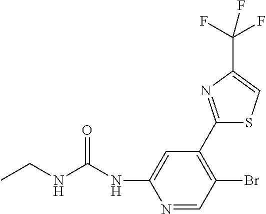

TFAA (1.128 mL, 7.99 mmol) followed by TEA (1.113 mL, 7.99 mmol) were added to a mixture of 1-(5-bromo-4-(4-hydroxy-4-(trifluoromethyl)-4,5-dihydrothiazol-2-yl)pyridin-2-yl)-3-ethylurea (Intermediate 4, 2.2 g, 5.32 mmol) in DCM (30 mL). The reaction mixture was allowed to stir overnight at room temperature. Another 150 uL of TEA and TFAA were added and the reaction mixture was stirred for additional 3 h. Then the reaction was concentrated under reduced pressure and the residue was partitioned between water and ethyl acetate. The layers were separated and the organic layer was washed with sodium bicarbonate solution, water and brine. The organic layer was dried over magnesium sulfate and concentrated under reduced pressure. The light yellow solid obtained was purified by normal phase chromatography (1% MeOH in dichloromethane to 3% MeOH in dichloromethane) to give the desired product (617 mg). MS (ESP): 396 (M+1) for C₁₂H₁₀BrN₃O; NMR: 1.07 (t, 3H); 3.11-3.17 (m, 2H); 7.24 (t, 1H); 8.35 (s, 1H); 8.50 (s, 1H); 8.77 (s, 1H); 9.34 (s, 1H).

Intermediate 4

### 1-(5-Bromo-4-(4-hydroxy-4-(trifluoromethyl)-4,5-dihydrothiazol-2-yl)pyridin-2-yl)-3-ethylurea

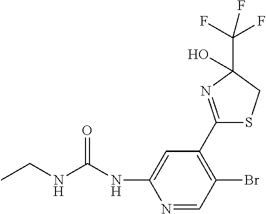

To a mixture of 5-bromo-2-(3-ethylureido)pyridine-4-carbothioamide (Intermediate 5, 1.1 g, 3.63 mmol) in acetonitrile (25 mL), was added 3-bromo-1,1,1-trifluoropropan-2-one (2.260 mL, 21.77 mmol) and the reaction mixture was heated at 80° C. for 4 h. A clear solution resulted within an hour. The solution was then concentrated under reduced pressure and the resulting residue was partitioned between water and ethyl acetate. The organic layer was washed with water and brine, dried over magnesium sulfate and concentrated under reduced pressure to give light yellow solid, which, was purified by normal phase column chromatography (silica, 2% MeOH in dichloromethane to 5% MeOH in dichloromethane) to give white solid (470 mg). MS (ESP): 414 (M+1) for C₁₂H₁₂BrF₃N₄O₂S; NMR: 1.06 (t, 3H); 3.12-3.18 (m, 2H); 3.60 (dd, 1H); 3.90 (dd, 1H); 7.13 (brs, 1H); 7.98 (s, 1H); 8.47 (s, 1H); 9.41 (s, 1H).

### Intermediate 5

### 5-Bromo-2-(3-ethylureido)pyridine-4-carbothioamide

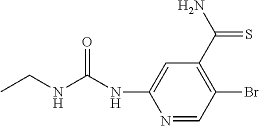

To a mixture of 5-bromo-2-(3-ethylureido)isonicotinamide (Intermediate 6, 1.25 g, 4.35 mmol) in THF (20 mL), was added Lawessons reagent (1.761 g, 4.35 mmol). The reaction mixture was then heated to 70° C. overnight. The solid that formed was collected by filtration and washed with THF to provide 1 g of desired product. MS (ESP): 304 (M+1) for C₁₉H₁₁BrN₄OS

### Intermediate 6

### 5-Bromo-2-(3-ethylureido)isonicotinamide

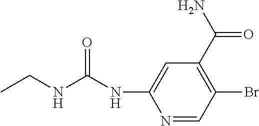

To a mixture of methyl 2-amino-5-bromoisonicotinate (3 g, 12.98 mmol) and chloroform (12 mL) in a microwave vial, isocyanatoethane (1.122 mL, 14.28 mmol) was added and the reaction mixture was heated at 110° C. for 3 h. The reaction mixture was concentrated under reduced pressure and 50 mL of 7N ammonia in MeOH was added. The resulting mixture was stirred at room temperature overnight, concentrated under reduced pressure and the resulting solid obtained was washed with acetonitrile to give a white solid (3.5 g).

MS (ESP): 287 (M+1) for C₁₉H₁₁BrN₄O₂

### Intermediate 7

### 1-(5′-(2-Acetylhydrazinecarbonyl)-4-(4-(trifluoromethyl)thiazol-2-yl)-3,3′-bipyridin-6-yl)-3-ethylurea

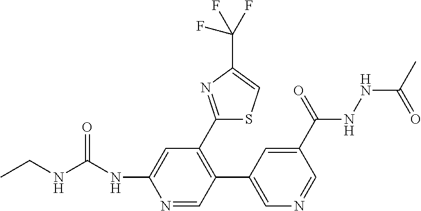

Triethylamine (0.054 mL, 0.39 mmol) and acetohydrazide (14.40 mg, 0.19 mmol) were added to a solution of 6′-(3-ethylureido)-4′-(4-(trifluoromethyl)thiazol-2-yl)-3,3′-bipyridine-5-carboxylic acid (Intermediate 1, 85 mg, 0.19 mmol) in DMF (1.5 mL). The mixture was stirred for 5 minutes and then HATU (89 mg, 0.23 mmol) was added. The resulting light yellow solution was stirred at room temperature for one hour then it was diluted with water. The aqueous layer was freeze dried and the solid obtained was extracted with THF and concentrated to give 184 mg of the crude product.

MS (ESP): 494 (M+1) for C₂₀H₁₈F₃N₇O₃S

### Intermediate 8

### 1-Ethyl-3-(5′-(2-isobutyrylhydrazinecarbonyl)-4-(4-(trifluoromethyl)thiazol-2-yl)-3,3′-bipyridin-6-yl)urea

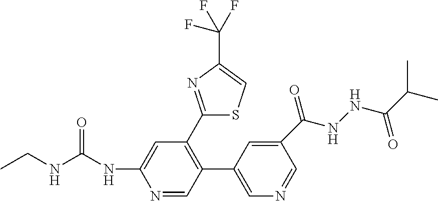

Intermediate 9 was synthesized according to the procedure described for Intermediate 7 using 6′-(3-ethylureido)-4′-(4-(trifluoromethyl)thiazol-2-yl)-3,3′-bipyridine-5-carboxylic acid and isobutylhydrazide as the starting material.

MS (ESP): 522 (M+1) for C₂₂H₂₂F₃N₇O₃S

¹H-NMR (DMSO-d₆) δ: 1.06 (d, 6H); 1.10 (t, 3H); 3.12-3.27 (m, 3H); 7.54 (brs, 1H); 8.19 (s, 1H); 8.24 (s, 1H); 8.36 (s, 1H); 8.56 (s, 1H); 8.64 (d, 1H); 9.04 (d, 1H); 9.49 (s, 1H); 9.94 (s, 1H); 10.54 (s, 1H).

### Intermediate 9

### 1-Ethyl-3-(5′-(hydrazinecarbonyl)-4-(4-(trifluoromethyl)thiazol-2-yl)-3,3′-bipyridin-6-yl)urea

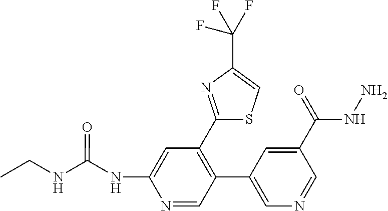

Ethyl 6′-(3-ethylureido)-4′-(4-(trifluoromethyl)thiazol-2-yl)-3,3′-bipyridine-5-carboxylate (Intermediate 2, 150 mg, 0.32 mmol) and hydrazine hydrate (31 mg, 0.97 mmol) were taken in ethanol (6 ml) and heated at 80° C. for 5 h. The reaction was cooled down and concentrated to give tan colored solid that was washed with 10% MeOH in dichloromethane and dried to give the title compound (101 mg).

MS (ESP): 452 (M+1) for C₁₈H₁₆F₃N₇O₃S

¹H-NMR (DMSO-d₆) δ: 1.09 (t, 3H); 3.10-3.25 (m, 2H); 4.57 (brs, 2H); 7.55 (brs, 1H); 8.13 (s, 1H); 8.23 (s, 1H); 8.34 (s, 1H); 8.55 (s, 1H); 8.59 (s, 1H); 8.99 (s, 1H); 9.48 (s, 1H); 9.97 (s, 1H).

### Intermediate 10

### N-(1-(Dimethylamino)ethylidene)-6′-(3-ethylureido)-4′-(4-(trifluoromethyl)thiazol-2-yl)-3,3′-bipyridine-5-carboxamide

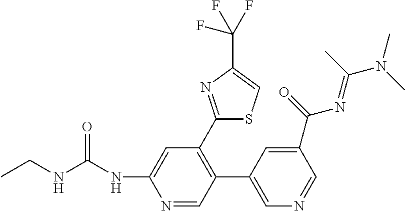

A mixture of 6′-(3-ethylureido)-4′-(4-(trifluoromethyl)thiazol-2-yl)-3,3′-bipyridine-5-carboxamide (Intermediate 11, 270 mg, 0.62 mmol) in 1,1-dimethoxy-N,N-dimethylethanamine (10 mL, 68.40 mmol) was heated to 120° C. for one hour and cooled down. The solid was filtered off and washed with acetonitrile and dried to give product as off-white solid (178 mg).

MS (ESP): 506 (M+1) for C₂₂H₂₂F₃N₇O₂S

¹H-NMR (DMSO-d₆) δ: 1.10 (t, 3H); 2.29 (s, 3H); 3.11 (s, 3H); 3.14 (s, 3H); 3.15-3.28 (m, 2H); 7.60 (brs, 1H); 8.14 (s, 1H); 8.20 (s, 1H); 8.37 (s, 1H); 8.55 (s, 1H); 8.63 (d, 1H); 9.16 (d, 1H); 9.48 (s, 1H).

### Intermediate 11

### 6′-(3-Ethylureido)-4′-(4-(trifluoromethyl)thiazol-2-yl)-3,3′-bipyridine-5-carboxamide

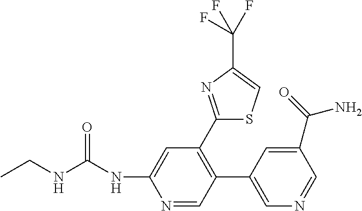

Triethylamine (0.040 mL, 0.29 mmol) and 2-phenylpropan-2-amine (19.47 mg, 0.14 mmol) were added to a solution of 6′-(3-ethylureido)-4′-(4-(trifluoromethyl)thiazol-2-yl)-3,3′-bipyridine-5-carboxylic acid (Intermediate 1, 63 mg, 0.14 mmol) in DMF (1.5 mL). The reaction solution was stirred for 5 minutes and then 2-(3H-[1,2,3]triazolo[4,5-b]pyridin-3-yl)-1,1,3,3-tetramethylisouronium hexafluorophosphate(V) (54.8 mg, 0.14 mmol) were added. The resulting light yellow solution was stirred at room temperature for 30 min. The desired product was precipitated with water and collected via filtration and dried to give an off-white solid (62 mg). The precipitate was taken in TFA (2 mL) and stirred overnight at room temperature and at 40° C. for another 3 h. The reaction was concentrated under reduced pressure and the residue was taken up in ethyl acetate and washed with sodium bicarbonate solution, water and brine. It was then dried over magnesium sulfate and concentrated to give white solid that was triturated with acetonitrile and dried to give the product (33 mg).

MS (ESP): 437 (M+1) for C₁₈H₁₅F₃N₆O₂S

¹H-NMR (DMSO-d₆: 1.09 (t, 3H); 3.18-3.24 (m, 2H); 7.45 (br s, 1H); 7.65 (s, 1H); 8.16 (s, 1H); 8.18 (s, 1H); 8.24 (s, 1H); 8.35 (s, 1H); 8.55 (d, 1H); 8.60 (d, 1H); 9.05 (s, 1H); 9.49 (s, 1H).

### Intermediate 12

### 1-Ethyl-3-(5-(4,4,5,5-tetramethyl-1,3,2-dioxaborolan-2-yl)-4-(4-(trifluoromethyl)thiazol-2-yl)pyridin-2-yl)urea

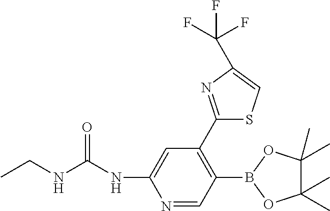

1-(5-Bromo-4-(4-(trifluoromethyl)thiazol-2-yl)pyridin-2-yl)-3-ethylurea (Intermediate 3, 200 mg, 0.51 mmol), 4,4,4′,4′,5,5,5′,5′-octamethyl-2,2′-bi(1,3,2-dioxaborolane) (386 mg, 1.52 mmol), potassium acetate (149 mg, 1.52 mmol), and 1,1′-bis(diphenylphosphino)ferrocene-palladium dichloride (20.72 mg, 0.03 mmol) were taken in a microwave vial and degassed with argon. DMSO (4 mL) was added to the vial and the solution was heated at 90° C. for 5 h. The reaction mixture was partitioned between water and ethyl acetate. The layers were separated and the organic layer was back extracted three times with ethyl acetate. The organic layers were combined and washed with water and brine, then dried over magnesium sulfate and concentrated under reduced pressure to give a light brown solid that was a mixture of the title compound (35%), {6-{[(ethylamino)carbonyl]amino}-4-[4-(trifluoromethyl)-1,3-thiazol-2-yl]pyridin-3-yl}boronic acid (25%) and N-ethyl-N-{4-[4-(trifluoromethyl)-1,3-thiazol-2-yl]pyridin-2-yl}urea (25%). The crude mixture was taken to the next step without further purification.

### Intermediate 13

### Methyl 2-(6-(3-ethylureido)-4-(4-(trifluoromethyl)thiazol-2-yl)pyridin-3-yl)thiazole-5-carboxylate

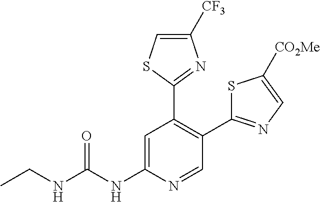

In a microwave reaction vessel, 1-ethyl-3-(5-(4,4,5,5-tetramethyl-1,3,2-dioxaborolan-2-yl)-4-(4-(trifluoromethyl)thiazol-2-yl)pyridin-2-yl)urea (Intermediate 12, 415 mg, 0.94 mmol), methyl 2-bromothiazole-5-carboxylate (208 mg, 0.94 mmol) and cesium carbonate (119 mg, 1.13 mmol) were combined and suspended in dioxane/water. Pd(PPh₃)₄ (54.2 mg, 0.05 mmol) was added in a single portion. The vessel was sealed and heated to 100° C. in the microwave for 60 minutes, then diluted with water and EtOAc. The aqueous and organic layers were separated, and the organic was dried over Na₂SO₄, filter and concentrate. Solids precipitated from solution upon concentrating and were collected and washed with minimal CH₂Cl₂. Analysis showed the solids to the desired reaction product. The mother liquor was concentrated further and the crude product was purified by flash column chromatography (0-100% EtOAc/hexanes). Isolation gave 128 mg of the title compound.

MS (ESP): 458 (M+1) for C₁₇H₁₄F₃N₅O₃S₂.

Intermediate 14

### N-(4-Bromopyridin-2-yl)-N′-ethylurea

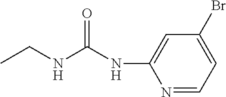

Isocyanatoethane (0.913 mL, 11.56 mmol) was added to a mixture of 4-bromopyridin-2-amine (2 g, 11.56 mmol) in chloroform (10 mL), and the mixture was heated at 110° C. for 2 h. The reaction mixture was concentrated under reduced pressure and triturated with acetonitrile to give a white solid (2.15 g).

MS (ESP): 243 (M+1) for C₈H₁₀BrN₃O

¹H-NMR (DMSO-d₆) δ: 1.08 (t, 3H); 3.12-3.18 (m, 2H); 7.16 (dd, 1H); 7.65 (brs, 1H); 7.74 (s, 1H); 8.07 (d, 1H); 9.29 (s, 1H)

### Intermediate 15

### N-[5-bromo-4-(4-pyridin-2-yl-1,3-thiazol-2-yl)pyridin-2-yl]-N′-ethylurea

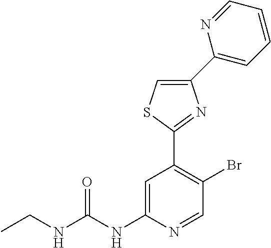

2-Bromo-1-pyridin-2-ylethanone (0.463 g, 1.65 mmol) was added to a mixture of 5-bromo-2-(3-ethylureido)pyridine-4-carbothioamide (Intermediate 5, 0.5 g, 1.65 mmol) in acetonitrile (3 mL), and the reaction mixture was heated to 80° for six hours. The reaction mixture was cooled to room temperature and concentrated under reduced pressure. The crude reaction mixture was purified by column chromatography (Silica gel, 10% MeOH in CH₂Cl₂).

Isolation gave 670 mg of the title compound as an off-white solid. LC/MS (ES⁺)[(M+H)⁺]: 404, 406 for C₁₆H₁₄BrN₅OS.

¹H NMR (300 MHz, d₆-DMSO): 1.08 (t, 3H), 3.18 (m, 2H), 7.33 (t, 1H), 7.42 (m, 1H), 7.98 (m, 1H), 8.16 (m, 1H), 8.51 (s, 1H), 8.55 (s, 1H), 8.59 (s, 1H), 8.67 (s, 1H), 9.39 (s, 1H).

### Intermediate 16

### N-[5-bromo-4-(4-phenyl-1,3-thiazol-2-yl)pyridin-2-yl]-N′-ethylurea

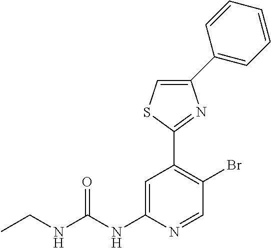

2-Bromo-1-phenylethanone (0.105 g, 0.53 mmol) was added to a mixture of 5-bromo-2-(3-ethylureido)pyridine-4-carbothioamide (Intermediate 5, 0.146 g, 0.48 mmol) in acetonitrile (3 mL), and the reaction mixture was heated to 80° for 16 h. The reaction mixture was cooled to room temperature and concentrated under reduced pressure. The resulting solids were filtered and washed with acetonitrile. Isolation gave 164 mg of the title compound as an off-white solid.

LC/MS (ES⁺)[(M+H)⁺]: 403, 405 for C₁₇H₁₅BrN₄OS.

¹H NMR (300 MHz, d₆-DMSO): 1.08 (t, 3H); 3.04-3.28 (m, 2H); 7.36 (m, 1H); 7.45 (m, 1H); 7.50 (t, 2H); 8.02-8.10 (m, 2H); 8.47 (s, 1H); 8.50 (s, 1H); 8.53 (s, 1H); 9.39 (s, 1H).

Intermediate 17

### 1-(4-(benzo[d]thiazol-2-yl)pyridin-2-yl)-3-ethylurea

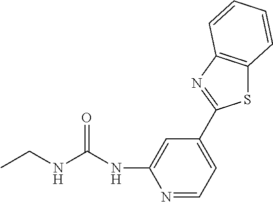

1-(4-bromopyridin-2-yl)-3-ethylurea (Intermediate 14, 0.50 g, 2.05 mmol), copper(I) iodide (0.039 g, 0.20 mmol), and Pd(PPh₃)₄ (0.118 g, 0.10 mmol) were combined in a microwave vial and degassed with nitrogen. DMF (4 mL) was added to the vial followed by slow addition of 2-(tributylstannyl)benzo[d]thiazole (1.130 g, 2.66 mmol), and the reaction mixture was heated to 100° C. for 60 minutes. The reaction mixture was partitioned between water and ethyl acetate and layers separated. The organic layer was washed with saturated NaHCO₃, water, brine and dried over magnesium sulfate, and concentrated. The resulting solids were filtered and then washed with acetonitrile followed by chloroform to yield 140 mg of the title compound as an off-white solid.

LC/MS (ES⁺)[(M+H)⁺]: 299 for C₁₅H₁₄N₄OS.

¹H NMR (300 MHz, d₆-DMSO): 1.11 (t, 3H), 3.21 (m, 2H), 7.54 (t, 1H), 7.56 (d, 1H), 7.61 (m, 1H), 7.76 (t, 1H), 8.15 (d, 1H), 8.21 (d, 1H), 8.23 (s, 1H), 8.35 (d, 1H), 9.39 (s, 1H).

### Intermediate 18

### 1-(4-(benzo[d]thiazol-2-yl)-5-bromopyridin-2-yl)-3-ethylurea

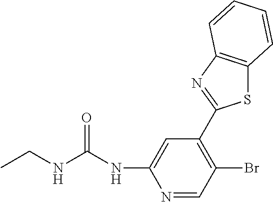

In a 25 mL pear-shaped flask 1-(4-(benzo[d]thiazol-2-yl)pyridin-2-yl)-3-ethylurea (Intermediate 17, 144 mg, 0.48 mmol) and 1-bromopyrrolidine-2,5-dione (96 mg, 0.54 mmol) were suspended in DMF (2 mL). The reaction mixture was heated to 80° C. for 4 hrs. The reaction was partitioned between water and ethyl acetate. The layers were separated and the organic layer was washed with a 5% sodium thiosulfate solution, followed by water and brine, then dried over magnesium sulfate and concentrated. The solids were tichurated with acetonitrile, filtered, washed and dried in vacuo. Isolation gave 160 mg of the title compound as an off-white solid.

LC/MS (ES⁺)[(M+H)⁺]: 377, 379 for C₁₈H₁₃BrN₄OS.

¹H NMR (300 MHz, d₆-DMSO): 1.09 (t, 3H), 3.17 (m, 2H), 7.27 (t, 1H), 7.58 (t, 1H), 7.65 (t, 1H), 8.19 (d, 1H), 8.27 (d, 1H), 8.42 (s, 1H), 8.58 (s, 1H), 9.44 (s, 1H).

### Intermediate 19

### Ethyl 6′-{[(ethylamino)carbonyl]amino}-4′-(4-pyridin-2-yl-1,3-thiazol-2-yl)-3,3′-bipyridine-5-carboxylate

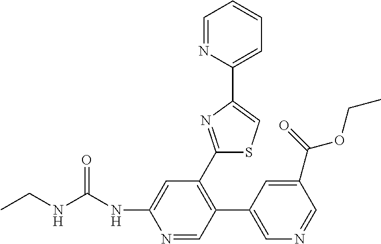

In a microwave vessel, N-[5-bromo-4-(4-pyridin-2-yl-1,3-thiazol-2-yl)pyridin-2-yl]-N′-ethylurea (Intermediate 15, 0.1 g, 0.25 mmol), ethyl 5-(4,4,5,5-tetramethyl-1,3,2-dioxaborolan-2-yl)nicotinate (0.103 g, 0.37 mmol) and cesium carbonate (0.121 g, 0.37 mmol) were combined and suspended in a mixture of dioxane and water (4:1; 2.5 mL/0.5 mL). The suspension was degassed and purged with nitrogen. Pd(PPh₃)₄ (0.014 g, 0.01 mmol) was added and the mixture was degassed and purged a second time. The reaction mixture was heated in the microwave at 100° C. for 60 minutes. The reaction was partitioned between water and ethyl acetate. The layers were separated, and the organic phase was washed with saturated NaHCO₃, water and brine, then dried over magnesium sulfate and concentrated. The resulting solids were filtered, then washed with acetonitrile followed by chloroform. Isolation gave 80 mg of the title compound as an off-white solid.

LC/MS (ES⁺)[(M+H)⁺]: 475 for C₂₄H₂₂N₆O₃S.

¹H NMR (300 MHz, CHCl₃): 1.11 (t, 3H), 1.25 (t, 3H), 3.21 (m, 2H), 4.29 (m, 2H), 7.35 (m, 1H), 7.60 (s, 1H), 7.63 (s, 1H), 7.82 (m, 1H), 8.26 (m, 2H), 8.34 (s, 1H), 8.36 (s, 1H), 8.60 (m, 1H), 8.80 (d, 1H), 9.10 (d, 1H), 9.49 (s, 1H).

### Intermediate 20

### Ethyl 6′-{[(ethylamino)carbonyl]amino}-4′-(4-phenyl-1,3-thiazol-2-yl)-3,3′-bipyridine-5-carboxylate

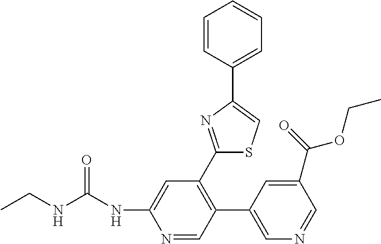

In a microwave vessel, N-[5-bromo-4-(4-phenyl-1,3-thiazol-2-yl)pyridin-2-yl]-N′-ethylurea (Intermediate 16, 0.17 g, 0.42 mmol), ethyl 5-(4,4,5,5-tetramethyl-1,3,2-dioxaborolan-2-yl)nicotinate (0.14 g, 0.51 mmol) and cesium carbonate (0.165 g, 0.51 mmol) were combined and suspended in a mixture of dioxane and water (4:1; 2.5 mL/0.5 mL). The suspension was degassed and purged with nitrogen. Pd(PPh₃)₄ (0.024 g, 0.02 mmol) was added and the mixture was degassed and purged a second time. The reaction mixture was heated in the microwave at 100° C. for 60 minutes. The reaction was partitioned between water and ethyl acetate, the layers were separated, and the organic phase was washed with saturated NaHCO₃, water and brine, then dried over magnesium sulfate, and concentrated. The resulting solids were filtered, washed with acetonitrile followed by chloroform. Isolation gave 200 mg of the title compound as an off-white solid.

LC/MS (ES⁺)[(M+H)⁺]: 474 for C₂₅H₂₃N₅O₃S;

¹H NMR (300 MHz, CHCl₃): 1.10 (t, 3H), 1.26 (t, 3H), 3.21 (m, 2H), 4.30 (q, 2H), 7.25 (t, 1H), 7.63 (t, 1H), 7.35 (s, 1H), 7.38 (s, 1H), 7.68 (d, 1H), 7.71 (d, 1H), 8.22 (s, 1H), 8.24 (s, 1H), 8.26 (t, 1H), 8.32 (t, 1H), 8.77 (d, 1H); 9.10 (d, 1H), 9.48 (s, 1H).

### Intermediate 21

### ethyl 4′-(benzo[d]thiazol-2-yl)-6′-(3-ethylureido)-3,3′-bipyridine-5-carboxylate

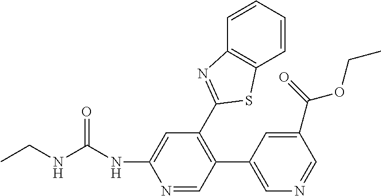

Intermediate 21 was synthesized as described for Intermediate 20 from Intermediate 18 and ethyl 5-(4,4,5,5-tetramethyl-1,3,2-dioxaborolan-2-yl)nicotinate.

LC/MS (ES⁺)[(M+H)⁺]: 448 for C₂₃H₂₁N₅O₃S.

¹H NMR (300 MHz, d₆-DMSO): 1.11 (t, 3H), 1.25 (t, 3H), 3.21 (m, 2H), 4.29 (q, 2H), 7.47 (m, 1H), 7.54 (m, 1H), 7.60 (t, 1H), 7.98 (m, 1H), 8.09 (m, 1H), 8.24 (t, 1H), 8.27 (s, 1H), 8.41 (s, 1H), 8.74 (d, 1H), 9.07 (d, 1H), 9.54 (s, 1H).

### Intermediate 22

### N-ethyl-N′[5′-(hydrazinocarbonyl)-4-(4-pyridin-2-yl-1,3-thiazol-2-yl)-3,3′-bipyridin-6-yl]urea

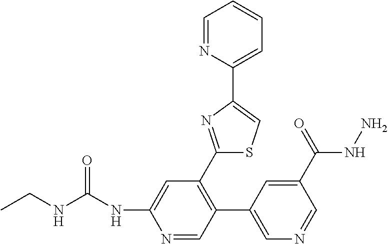

In a 25 mL round-bottom flask, ethyl 6′-{[(ethylamino)carbonyl]amino}-4′-(4-pyridin-2-yl-1,3-thiazol-2-yl)-3,3′-bipyridine-5-carboxylate (Intermediate 19, 0.26 g, 0.55 mmol) and hydrazine hydrate (0.165 g, 3.29 mmol) were mixed in ethanol (6 mL), and stirred at 80° C. overnight. The reaction mixture was cooled to room temperature and concentrated under reduced pressure. The resulting residue was tichurated with 10% MeOH in DCM. The resulting solid was filtered, washed and dried in vacuo. Isolation gave 250 mg of the title compound.

LC/MS (ES⁺)[(M+H)⁺]: 461 for C₂₂H₂₀N₈O₂S.

¹H NMR (300 MHz, d₆-DMSO): 1.11 (t, 3H), 3.22 (m, 2H), 4.58 (s, 2H), 7.36 (m, 1H), 7.68 (s, 1H), 7.70 (s, 1H), 7.86 (m, 1H), 8.21 (t, 1H), 8.32 (s, 1H), 8.33 (s, 1H), 8.36 (s, 1H), 8.60 (m, 1H), 8.64 (d, 1H), 9.0 (d, 1H), 9.52 (s, 1H), 10.02 (s, 1H).

### Intermediate 23

### 1-ethyl-3-(5′-(hydrazinecarbonyl)-4-(4-phenylthiazol-2-yl)-3,3′-bipyridin-6-yl)urea

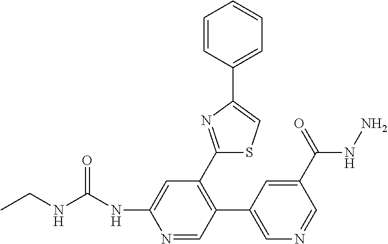

Intermediates 23 was synthesized according to the procedure described for Intermediate 22 from Intermediate 20 and hydrazine.

LC/MS (ES⁺)[(M+H)⁺]: 460 for C₂₃H₂₁N₇O₂S.

¹H NMR (300 MHz, d₆-DMSO): 1.12 (t, 3H), 3.22 (m, 2H), 4.58 (s, 2H), 7.34-7.43 (m, 3H), 7.64 (t, 1H), 7.74 (d, 1H), 7.76 (d, 1H), 8.20 (t, 1H), 8.23 (s, 1H), 8.28 (s, 1H), 8.32 (s, 1H), 8.63 (d, 1H), 9.01 (d, 1H), 9.48 (s, 1H), 10.01 (s, 1H).

### Intermediate 24

### 1-(4-(benzo[d]thiazol-2-yl)-5′-(hydrazinecarbonyl)-3,3′-bipyridin-6-yl)-3-ethylurea

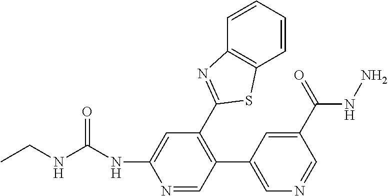

Intermediates 24 was synthesized according to the procedure described for Intermediate 22 from Intermediate 21 and hydrazine

LC/MS (ES⁺)[(M+H)⁺]: 434 for C₂₁H₁₉N₇O₂S.

¹H NMR (300 MHz, CHCl₃): 1.10 (t, 3H), 3.16 (m, 2H), 4.55 (s, 2H), 7.48 (m, 1H), 7.54 (m, 1H), 7.57 (m, 1H), 7.98 (d, 1H), 8.09 (d, 1H), 8.18 (t, 1H), 8.28 (s, 1H), 8.38 (s, 1H), 8.58 (d, 1H), 8.97 (d, 1H), 9.52 (s, 1H), 9.98 (s, 1H).

### Intermediate 25

### diethyl 2-{6-[(ethylcarbamoyl)amino]-4-[4-(trifluoromethyl)-1,3-thiazol-2-yl]pyridin-3-yl}-1,3-thiazole-4,5-dicarboxylate

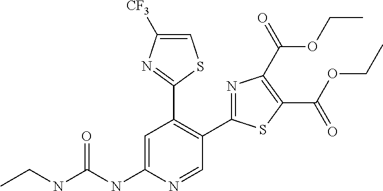

A slurry of crude 1-ethyl-3-(5-(4,4,5,5-tetramethyl-1,3,2-dioxaborolan-2-yl)-4-(4-(trifluoromethyl)thiazol-2-yl)pyridin-2-yl)urea (Intermediate 12, 110 mg, 0.25 mmol), diethyl 2-chlorothiazole-4,5-dicarboxylate (WO2006087543, 66 mg, 0.25 mmol) and K₂CO₃ (86 mg, 0.625 mmol) in 1,4-dioxane-water (8+3 ml) was purged with nitrogen for 30 min at room temperature. Bis(triphenylphosphine)palladium dichloride (18 mg, 0.025 mmol) was added and the resulting mixture was stirred at 80-90° C. for 1.2 h. The reaction mixture was cooled, diluted with water (10 mL), and extracted with EtOAc (2×80 mL). The combined extracts were dried over sodium sulfate and concentrated to a residue under reduced pressure. The residue was purified via flash chromatography (50% EtOAc-heptane+10% EtOH) to afford 90 mg (67%) of desired product as light brown gum.

MS (ESP): 544 (M+H⁺) for C₂₁H₂₀F₃N₅O₅S₂

### Intermediate 26

### 5-(5-Bromopyridin-3-yl)-1H-pyrazol-3(2H)-one

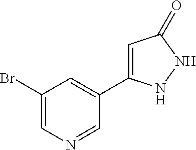

Hydrazine hydrate (0.110 mL, 3.49 mmol) was added to a mixture of methyl 3-(5-bromopyridin-3-yl)-3-oxopropanoate (300 mg, 1.16 mmol) in methanol (5 mL). The resulting mixture was heated at reflux for 2 h. The reaction mixture was cooled to room temperature and the solid that formed was collected by filtration. The solid was washed with methanol and dried under vacuum to give the title compound as an off-white solid.

MS (ESP): 239 (M−1) for C₈H₆BrN₃

¹H-NMR (DMSO-d₆) δ: 6.12 (brs, 1H); 8.32 (s, 1H); 8.60 (s, 1H); 8.90 (s, 1H); 9.88 (br s, 1H); 12.33 (br s, 1H).

### Intermediate 27

### 6′-(3-Ethylureido)-N′-hydroxy-4′-(4-(trifluoromethyl)thiazol-2-yl)-3,3′-bipyridine-5-carboximidamide

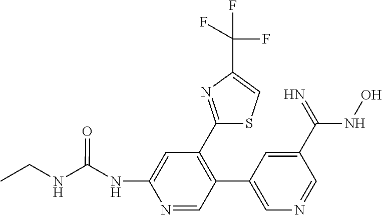

Hydroxylamine (0.040 mL, 0.65 mmol) (50% in water) was added to a suspension of 1-(5′-cyano-4-(4-(trifluoromethyl)thiazol-2-yl)-3,3′-bipyridin-6-yl)-3-ethylurea (Example 2, 180 mg, 0.43 mmol) in ethanol (10 mL), and the mixture was heated to 80° C. for 1.5 hours. The reaction mixture was concentrated under reduced pressure and the resulting residue was triturated with acetonitrile to give the title compound as a tan colored solid (180 mg).

MS (ESP): 452 (M+1) for C₁₉H₁₄F₃N₇O₃S

### Intermediate 28

### 2-Bromo-1-(1-methyl-1H-pyrazol-4-yl)ethanone

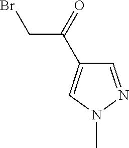

In a 25 mL flask, 1-(1-methyl-1H-pyrazol-4-yl)ethanone (0.602 g, 4.85 mmol) was dissolved in chloroform (20 mL). The colorless solution was made acidic with the addition of a few drops of HBr in acetic acid (3.92 mg, 0.05 mmol). A chloroform solution containing Br₂ (0.262 mL, 5.09 mmol) was added dropwise via an addition funnel. The reaction mixture was stirred at room temperature for 1 h, and then concentrated under reduced pressure. The crude solid was triturated in ethyl acetate, filtered, and dried in vacuo. The free base was obtained by triturating the product in 5% NaHCO₃ for 2 h. The solid was collected by filtration, washed with water, isopropyl alcohol and then dried in vacuo. Isolation gave 874 mg of the title compound.

LC/MS (ES⁺)[(M+H)⁺]: 204 for C₆H₇BrN₂O.

¹H NMR (300 MHz, d₆-DMSO): 3.88 (s, 3H), 4.56 (s, 2H), 7.99 (s, 1H), 8.47 (s, 1H).

### Intermediate 29

### 1-(5-bromo-4-(4-(1-methyl-1H-pyrazol-4-yl)thiazol-2-yl)pyridin-2-yl)-3-ethylurea

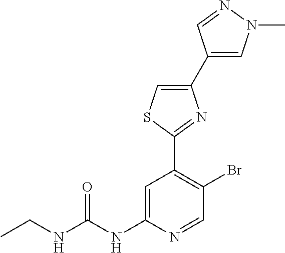

In a 25 mL flask 5-bromo-2-(3-ethylureido)pyridine-4-carbothioamide (Intermediate 5, 478 mg, 1.58 mmol) and 2-bromo-1-(1-methyl-1H-pyrazol-4-yl)ethanone (Intermediate 28, 352 mg, 1.73 mmol) were suspended in EtOH (10 mL). The reaction mixture was heated at 80° C. for 12 h. The reaction mixture was cooled to room temperature and concentrated under reduced pressure. The resulting solid was collected by filtration and washed with acetonitrile. Isolation gave 640 mg of the title compound as an off-white solid.

LC/MS (ES⁺)[(M+H)⁺]: 407, 409 for C₁₅H₁₅BrN₆OS.

¹H NMR (300 MHz, d₆-DMSO): 1.08 (t, 3H), 3.18 (m, 2H), 3.9 (s, 3H), 7.34 (m, 1H), 7.91 (s, 1H), 8.03 (s, 1H), 8.17 (s, 1H), 8.41 (s, 1H), 8.52 (s, 1H), 9.37 (s, 1H).

### Intermediate 30

The following Intermediate was prepared according to the procedure described for Intermediate 29 using the starting materials indicated.

| Int | Compound | Structure | Data | SM |
|---|---|---|---|---|
| 30 | 1-(5-Bromo- 4-(4-(pyridin- 4-yl)thiazol-2- yl)pyridin-2- yl)-3- ethylurea | 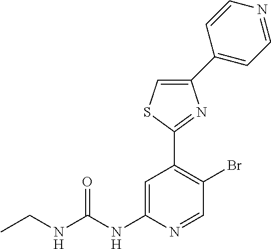 | LC/MS (ES⁺)[(M + H)⁺]: 404, 406 for C₁₆H₁₄BrN₅OS. ¹H NMR (300 MHz, d₆- DMSO): 1.09 (t, 3H), 3.16 (m, 2H), 7.21 (m, 1H), 8.53 (m, 1H), 8.55 (s, 2H) 8.59 (s, 1H), 9.0 (s, 1H), 9.02 (s, 1H), 9.23 (s, 1H), 9.42 (s, 1H). | Intermediate 5 and 2-bromo-1- pyridin-4- ylethanone |

### Intermediate 31

### 1-ethyl-3-(4-(1-methyl-1H-pyrazol-5-yl)pyridin-2-yl)urea

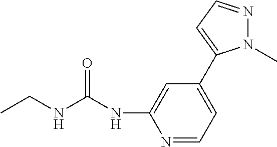

In a pear-shaped flask, 1-(4-bromopyridin-2-yl)-3-ethylurea (Intermediate 14, 0.3 g, 1.23 mmol), 1-methyl-5-(4,4,5,5-tetramethyl-1,3,2-dioxaborolan-2-yl)-1H-pyrazole (0.281 g, 1.35 mmol), Pd₂(dba)₃ (0.113 g, 0.12 mmol), 2-dicyclohexylphosphino-2′,4′,6′-tri-iso-propyl-1,1′-biphenyl (0.176 g, 0.37 mmol), Na₂CO₃ (0.156 g, 1.47 mmol) were combined and suspended in a mixture of acetonitrile and water (5:1; 7 mL/1.4 mL). The suspension was degassed and purged with nitrogen. The reaction mixture was heated at 90° C. for 60 min., then concentrated under reduced pressure, and partitioned between water and ethyl acetate. The organic phase was washed with water and brine, and then dried over magnesium sulfate. The mother liquor was concentrated under reduced pressure. The resulting solid was filtered and washed with acetonitrile. Isolation gave 146 mg of the title compound as an off-white solid.

LC/MS (ES⁺)[(M+H)⁺]: 246 for C₁₂H₁₅N₅O.

¹H NMR (300 MHz, d₆-DMSO): 1.08 (t, 3H), 3.19 (m, 2H), 3.9 (s, 3H), 6.53 (d, 1H), 7.11 (m, 1H), 7.51 (d, 1H), 7.56 (s, 1H), 7.97 (m, 1H), 8.26 (d, 1H), 9.26 (s, 1H).

### Intermediate 32

The following intermediate was prepared in accordance to the procedure described for Intermediate 18 using the starting materials indicated in the table.

| Int | Compound | Structure | Data | SM |
|---|---|---|---|---|
| 33 | 1-(5-bromo-4- (1-methyl-1H- pyrazol-5- yl)pyridin-2- yl)-3- ethylurea | 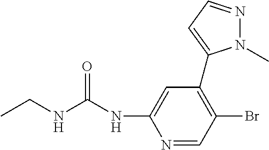 | LC/MS (ES⁺)[(M + H)⁺]: 324, 326 for C₁₂H₁₄BrN₅O. ¹H NMR (300 MHz, d₆- DMSO): 1.08 (t, 3H), 3.18 (m, 2H), 3.80 (s, 3H), 7.07 (m, 1H), 7.55 (s, 1H), 7.70 (s, 1H), 7.85 (m, 1H), 8.34 (d, 1H), 9.34 (s, 1H). | Intermediate 31 and 1- bromopyrrolidine- 2,5-dione |

### Intermediate 33-35

The following intermediates were prepared in accordance to the procedure described for Intermediate 20 using the starting materials indicated in the table.

| Int | Compound | Structure | Data | SM |
|---|---|---|---|---|
| 33 | Ethyl 6′-(3- ethylureido)- 4′-(4-(1- methyl-1H- pyrazol-4- yl)thiazol-2- yl)-3,3′- bipyridine-5- carboxylate | 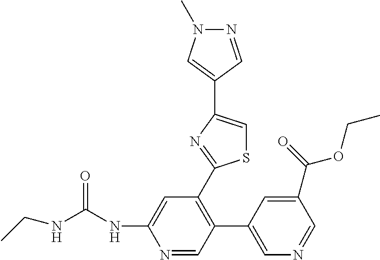 | LC/MS (ES⁺)[(M + H)⁺]: 478 for C₂₃H₂₃N₇O3S. ¹H NMR (300 MHz, d₆-DMSO): 1.10 (t, 3H), 1.29 (t, 3H), 3.21 (m, 2H), 3.83 (s, 3H), 4.32 (m, 2H), 7.60 (s, 1H), 7.61 (m, 1H), 7.76 (s, 1H), 7.92 (s, 1H), 8.17 (s, 1H), 8.24 (m, 1H), 8.31 (s, 1H), 8.74 (m, 1H), 9.09 (m, 1H), 9.47 (s, 1H). | Intermediate 29 and ethyl 5- (4,4,5,5- tetramethyl-1,3,2- dioxaborolan-2- yl)nicotinate, cesium carbonate, Pd(PPh₃)₄ |
| 34 | Ethyl 6′-(3- ethylureido)- 4′-(4-(pyridin- 4-yl)thiazol-2- yl)-3,3′- bipyridine-5- carboxylate | 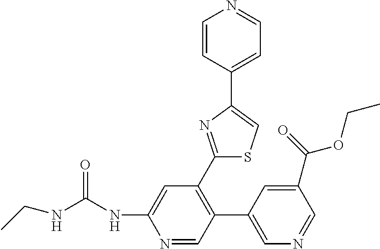 | LC/MS (ES⁺)[(M + H)⁺]: 475 for C₂₄H₂₂N₆O₃S. ¹H NMR (300 MHz, d₆-DMSO): 1.11 (t, 3H), 1.27 (t, 3H), 3.22 (m, 2H), 4.32 (m, 2H), 7.61 (m, 1H), 7.66 (m, 2H), 8.27 (m, 2H), 8.36 (s, 1H), 8.57 (s, 1H), 8.59 (m, 2H), 8.79 (d, 1H), 9.11 (d, 1H), 9.50 (s, 1H). | Intermediate 30 and ethyl 5- (4,4,5,5- tetramethyl-1,3,2- dioxaborolan-2- yl)nicotinate, cesium carbonate, Pd(PPh₃)₄ |
| 35 | ethyl 6′-(3- ethylureido)- 4′-(1-methyl- 1H-pyrazol-5- yl)-3,3′- bipyridine-5- carboxylate | 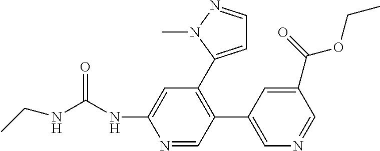 | LC/MS (ES⁺)[(M + H)⁺]: 395 forC₂₀H₂₂N₆O3. ¹H NMR(300 MHz, d₆- DMSO): 1.07 (t, 3H), 1.26 (t, 3H), 3.17 (m, 2H), 3.77 (s, 3H), 4.27 (m, 2H), 7.0 (m, 1H), 7.39 (s, 1H), 7.94 (m, 1H), 7.96 (m, 1H), 8.08 (s, 1H), 8.34 (m, 1H), 8.71 (m, 1H), 8.80 (m, 1H), 9.31 (s, 1H). | Intermediate 32 and ethyl 5- (4,4,5,5- tetramethyl-1,3,2- dioxaborolan-2- yl)nicotinate, cesium carbonate, Pd(PPh₃)₄ |

### Intermediates 36-37

The following intermediates were prepared in accordance to the procedure described for Intermediate 22 using the starting materials indicated in the table.

| Int | Compound | Structure | Data | SM |
|---|---|---|---|---|
| 36 | 1-ethyl-3-(5′- (hydrazinecar- bonyl)-4-(4-(1- methyl-1H- pyrazol-4- yl)thiazol-2- yl)-3,3′- bipyridin-6- yl)urea | 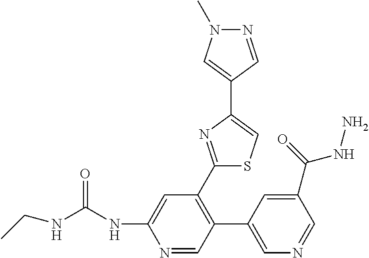 | LC/MS (ES⁺)[(M + H)⁺]: 464 for C₂₁H₂₁N₉O₂S. ¹H NMR (300 MHz, d₆- DMSO): 1.11 (t, 3H), 3.22 (m, 2H), 3.85 (s, 3H), 4.56 (m, 2H), 7.63 (m, 1H), 7.65 (s, 1H), 7.76 (s, 1H), 7.94 (s, 1H), 8.17 (m, 1H), 8.20 (m, 1H), 8.30 (s, 1H), 8.59 (d, 1H), 8.98 (d, 1H), 9.46 (s, 1H), 9.98 (s, 1H). | Intermediate 34 and hydrazine hydrate |
| 37 | 1-ethyl-3-(5′- (hydrazinecar- bonyl)-4-(1- methyl-1H- pyrazol-5-yl)- 3,3′-bipyridin- 6-yl)urea | 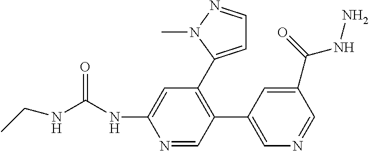 | LC/MS (ES⁺)[(M + H)⁺]: 381 for C₁₈H₂₀N₈O₂. | Intermediate 35 and hydrazine hydrate |

### Intermediate 38-42

The following intermediates were prepared in accordance to the procedure described for Intermediate 20 using the starting materials indicated in the table.

| Int | Compound | Structure | Data | SM |
|---|---|---|---|---|
| 38 | methyl 6-(3- ethylureido)- 4-(4- (trifluoro- methyl) thiazol-2- yl)-3,4′- bipyridine-2′- carboxylate | 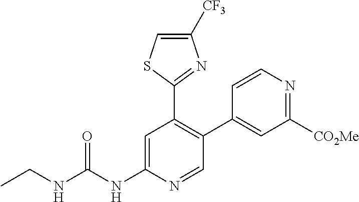 | LC/MS (ES⁺)[(M + H)⁺]: 452 for C₁₉H₁₆F₃N₅O₃S. ¹H NMR (300 MHz, CDCl3): 1.22 (t, 3H), 3.41 (m, 2H), 3.95 (s, 3H), 7.32 (d, 1H), 7.53 (s, 1H), 7.73 (s, 1H), 8.01 (s, 1H), 8.18 (s, 1H), 8.67 (d, 1H), 8.94 (broad s, 1H), 9.81 (broad s, 1H). | Intermediate 12 and methyl 4- bromopicolinate |
| 39 | Ethyl 2-(6-(3- ethylureido)- 4-(4- (trifluoro- methyl) thiazol-2- yl)pyridin-3- yl)-4- (pyrimidin-2- yl)thiazole-5- carboxylate | 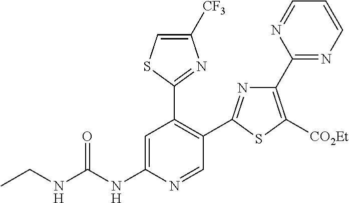 | LC/MS (ES⁺)[(M + H)⁺]: 550 for C₂₂H₁₈F₃N₇O₃S₂. ¹H NMR (300 MHz, d₆-DMSO): 1.01 (q, 6H), 3.14 (m, 2H), 4.08 (q, 2H), 7.39 (m, 1H), 7.52 (t, 1H), 8.08 (s, 1H), 8.68 (s, 1H), 8.72 (s, 1H), 8.84 (d, 2H), 9.66 (s, 1H). | Intermediate 12 and Intermediate 45 |
| 40 | methyl 2-(6- (3- ethylureido)- 4-(4- (trifluoro- methyl) thiazol-2- yl)pyridin-3- yl)-4-(1- methyl-1H- 1,2,4-triazol- 5-yl)thiazole- 5-carboxylate | 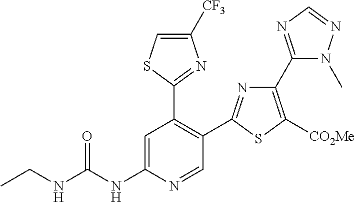 | LC/MS (ES⁺)[(M + H)⁺]: 539 for C₂₀H₁₇F₃N₈O₃S₂. ¹H NMR (300 MHz, d₆-DMSO): 1.03 (t, 3H), 3.11 (m, 2H), 3.62 (s, 3H), 3.70 (s, 3H), 7.52 (m, 1H), 8.00 (s, 1H), 8.05 (s, 1H), 8.67 (s, 1H), 8.72 (s, 1H), 9.67 (s, 1H). | Intermediate 12 and Intermediate 44 |
| 41 | methyl 2-(6- (3- ethylureido)- 4-(4- (trifluoro- methyl) thiazol-2- yl)pyridin-3- yl)-6- methyl- pyrimidine-4- carboxylate | 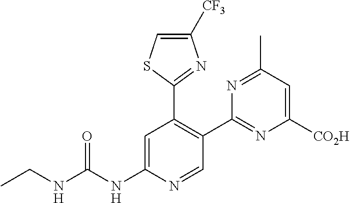 | LC/MS (ES⁺)[(M + H)⁺]: 453 for C₁₈H₁₅F₃N₆O₃S | Intermediate 12 and methyl 2- chloro-6- methylpyrimidine- 4-carboxylate |
| 42 | 6-(6-(3- ethylureido)- 4-(4- (trifluoro- methyl) thiazol-2- yl)pyridin-3- yl)pyrazine-2- carboxylic acid | 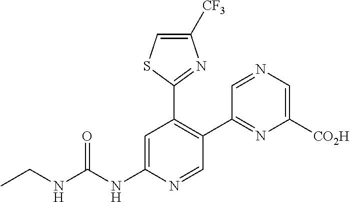 | LC/MS (ES⁺)[(M + H)⁺]: 439 for C₁₇H₁₃F₃N₆O₃S. | Intermediate 12 and 6- chloropyrazine-2- carboxylic acid |

### Intermediate 43

### Ethyl 2-chloro-4-pyrimidin-2-yl-1,3-thiazole-5-carboxylate

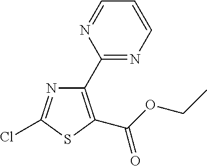

Ethyl 2-amino-4-pyrimidin-2-yl-1,3-thiazole-5-carboxylate (Intermediate 47; 0.55 g, 2.2 mmol) was suspended in glacial acetic acid (20 ml) and concentrated HCl (30 ml). The solution was cooled to 0° C. and a solution of sodium nitrite in water (15 ml) was added dropwise. After stirring at 0° C. for 10 mins, the reaction was slowly warmed to room temperature and stirred for 1 hour. The reaction was monitored by LCMS and once complete, a solution of urea (0.25 g) in water (10 ml) was added dropwise. After stirring at room temperature for 30 mins, solvent was removed under reduced pressure. The residue was partitioned with sat. NaHCO₃ (aq) and EtOAc. The layers were separated and the water layer was back extracted with EtOAc (×3). The combined organic layers were drying with MgSO₄ and concentrating yielded an orange oil which was used without purification (0.20 g).

MS (ES) (M+H)⁺: 270 for C₁₀H₈ClN₃O₂S.

### Intermediate 44

The following Intermediate was prepared according to the procedure described for Intermediate 43 from the starting materials indicated.

| Int | Compound | Data | SM |
|---|---|---|---|
| 44 | Methyl 2-chloro-4-(1-methyl- 1H-1,2,4-triazol-5-yl)-1,3- thiazole-5-carboxylate 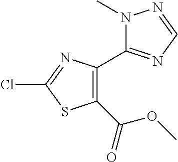 | MS (ES) (M + H)⁺: 259 for C₈H₇ClN₄O₂S NMR: 3.92 (s, 6H), 8.04 (s, 1H). | Intermediate 46 |

### Intermediate 45

### Ethyl 2-amino-4-pyrimidin-2-yl-1,3-thiazole-5-carboxylate

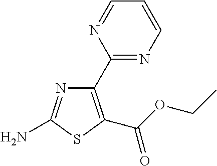

A suspension of ethyl 2-iodo-3-oxo-3-pyrimidin-2-ylpropanoate (Intermediate 47; 1.73 g, 5.4 mmol) and thiourea (0.62 g, 8.1 mmol) in EtOH was heated at reflux for 1 hour. After cooling to room temperature, the reaction was concentrated. The residue was suspended in water and basified with saturated aqueous Na₂CO₃. The resulting precipitate was filtered off and the filtrate was extracted with EtOAc (×3). The organic extracts were combined and dried with MgSO₄, then concentrated to an orange oil (0.55 g, 41%).

MS (ES) (M+H)⁺: 251 for C₁₀H₁₀N₄O₂S

NMR: 0.97 (t, 3H), 3.95 (q, 2H), 7.55 (t, 1H), 7.94 (s, 1H), 8.85 (d, 1H), 9.05 (d, 1H).

### Intermediate 46

The following Intermediate was synthesized according to the procedure described for Intermediate 45 from the starting materials indicated.

| Int | Compound | Data | SM |
|---|---|---|---|
| 46 | Methyl 2-amino-4-(1-methyl- 1H-1,2,4-triazol-5-yl)- 1,3-thiazole-5-carboxylate 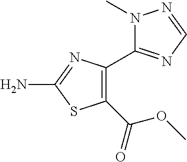 | MS (ES) (M + H)⁺: 240 for C₈H₉N₅O₂S NMR: 3.61 (s, 3H), 3.71 (s, 3H), 7.96 (s, 1H), 8.10 (s, 2H). | Intermediate 48 |

### Intermediate 47

### Ethyl 2-iodo-3-oxo-3-pyrimidin-2-ylpropanoate

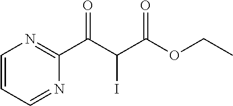

To a suspension of ethyl 3-oxo-3-pyrimidin-2-ylpropanoate (Intermediate 48; 1.19 g, 6.1 mmol) in EtOAc was added N-iodosuccinamide (1.38 g, 6.1 mmol) and Amberlyst-15 resin (1.19 g). After stirring at room temperature for 30 mins, LCMS shows a mixture of desired product and bis-iodinated product. The reaction mixture was filtered to remove the Amberlyst-15 resin, and the filtrate was concentrated to an orange oil which was then suspended in diethyl ether. The resulting precipitate was filtered and washed with ether. The filtrate was concentrated to an orange oil to provide the desired product (1.73 g, 89%).

MS (ES) (M+H)⁺: 321 for C₉H₉IN₂O₃

### Intermediate 48

### Ethyl 3-oxo-3-pyrimidin-2-ylpropanoate

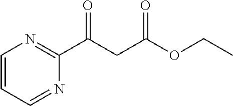

To a solution of pyrimidine-2-carboxylic acid (0.99 g, 7.98 mmol) in anhydrous THF (20 ml) was added carbonyl diimidazole (1.55 g, 9.57 mmol) and the suspension was heated at reflux for 2 hours. The mixture was then cooled to room temperature and used without workup or purification. In a separate flask, mono-ethyl malonate (0.94 ml, 7.98 mmol) was suspended in anhydrous THF (20 ml) and cooled to 0° C. Methyl magnesium bromide (5.32 ml, 15.96 mmol, 3.0 M in diethyl ether) was added dropwise. After stirring at 0° C. for 20 mins, the crude imidazolide solution prepared earlier was added slowly. The reaction was then heated at reflux overnight. After cooling to room temperature, the reaction mixture was diluted with water and acidified with concentrated HCl to pH 5. The solution was extracted with EtOAc (×3), dried with MgSO₄ and concentrated to a yellow oil (1.19 g, 77%). NMR showed a 2:1 mixture of the keto:enol forms.

MS (ES) (M+H)⁺: 195 for C₉H₁₀N₂O₃

NMR: 1.13-1.29 (t, 3H), 4.05-4.28 (q, 2H), 4.18 (s, 2H), 7.62-7.76 (t, 1H), 8.95-9.06 (d, 2H), 11.79 (s, 4H).

### Intermediate 49

### Methyl 3-(1-methyl-1H-1,2,4-triazol-5-yl)-3-oxopropanoate

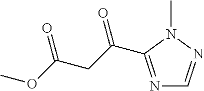

NaH (7.84 g, 196 mmol of a 60% dispersion in oil) was added portionwise to a solution of 6.18 g (34.5 mmol) of 1-(1-methyl-1H-1,2,4-triazol-5-yl)ethanone (Ohta, S.; Kawasaki, I.; Fukuno, A.; Yamashita, M.; Tada, T.; Kawabata, T. Chem. Pharm. Bull. (1993), 41(7), 1226-31) in 100 ml dimethylcarbonate. The mixture was heated to 90° C. for 2 hour forming a thick slurry. After cooling to room temperature, the mixture was slowly transferred to 1N HCl over ice. The pH of the mixture was brought to about 7 with NaHCO₃ before being saturated with NaCl and extracted 4 times with EtOAc. The EtOAc was dried (MgSO₄) and concentrated to give an oil that was chromatographed on silica gel (100% DCM followed by gradient elution to 50% EtOAc in DCM). The product (5.3 g) was obtained as an oil.

NMR: 3.78 (s, 3H), 4.11 (s, 2H), 4.22 (s, 3H), 7.94 (s, 1H).

### Intermediate 50

### 6-(3-ethylureido)-4-(4-(trifluoromethyl)thiazol-2-yl)-3,4′-bipyridine-2′-carboxylic acid

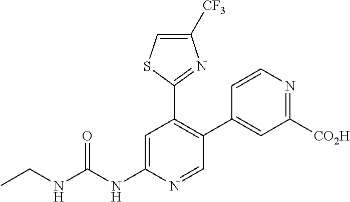

Methyl 6-(3-ethylureido)-4-(4-(trifluoromethyl)thiazol-2-yl)-3,4′-bipyridine-2′-carboxylate (Intermediate 38, 90 mg, 0.20 mmol) was dissolved in THF (2 mL) and methanol (2 mL). 1N LiOH (0.219 mL, 0.22 mmol) was added in a single portion, and the reaction mixture was heated to reflux for 15 min. The reaction mixture was cooled to room temperature and acidified with 2N HCl. The solid that precipitated was collected by filtration, washed with water and then dried in vacuo. Isolation gave 60 mg of the title compound.

LC/MS (ES⁺)[(M+H)⁺]: 438 for C₁₈H₁₄F₃N₅O₃S.

¹H NMR (300 MHz, d₆-DMSO): 1.09 (t, 3H), 3.19 (m, 2H), 7.53 (t, 1H), 7.56 (d, 1H), 7.86 (s, 1H), 8.14 (s, 1H), 8.37 (s, 1H), 8.59 (s, 1H), 8.68 (d, 1H), 9.53 (s, 1H).

### Intermediate 51

### 5-bromo-2-(3-ethylureido)isonicotinic acid

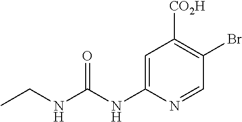

Methyl 5-bromo-2-(3-ethylureido)isonicotinate (Intermediate 6, 1 g, 3.31 mmol) was suspended in THF (5 mL) and methanol (5 mL). 1N LiOH (5 mL, 5.00 mmol) was added in a single portion, and the reaction was heated to reflux. The reaction mixture was cooled to room temperature and acidified with 2N HCl. The product precipitated from solution with the addition of water. The solid was collected by filtration and washed with water and dried in vacuo to give 843 mg of the title compound.

LC/MS (ES⁺[(M+H)⁺]: 288, 290 for C₉H₁₀BrN₃O₃.

¹H NMR (300 MHz, d₆-DMSO): 1.07 (t, 3H), 3.16 (m, 2H), 7.28 (t, 1H), 7.92 (s, 1H), 8.42 (s, 1H), 9.38 (s, 1H), 14.02 (broad s, 1H).

### Intermediate 52

### 1-(5-bromo-4-(2-isonicotinoylhydrazinecarbonyl)pyridin-2-yl)-3-ethylurea

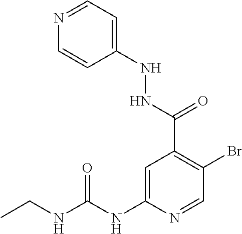

5-Bromo-2-(3-ethylureido)isonicotinic acid (Intermediate 51, 500 mg, 1.74 mmol) and HATU (792 mg, 2.08 mmol) were dissolved in DMF (5 mL) and DIEA (0.905 mL, 5.21 mmol). The solution was stirred for 5 min. Isonicotinohydrazide (238 mg, 1.74 mmol) was added in a single portion. The reaction mixture was diluted with water and acidified to pH 2 with 2N HCl. The solid that formed was collected by filtration, washed with water and dried in vacuo. Isolation gave 538 mg of the title compound.

LC/MS (ES⁺)[(M+H)⁺]: 407, 409 for C₁₅H₁₅BrN₆O₃.

¹H NMR (300 MHz, d₆-DMSO): 1.09 (t, 3H), 3.15 (m, 2H), 3.32 (d, 1H), 7.32 (m, 1H), 7.82 (m, 2H), 7.85 (s, 1H), 8.42 (s, 1H), 8.79 (m, 2H), 9.43 (s, 1H), 10.75-11.01 (d, 1H).

### Intermediate 53

### 1-(5-bromo-4-(5-(pyridin-4-yl)-1,3,4-oxadiazol-2-yl)pyridin-2-yl)-3-ethylurea

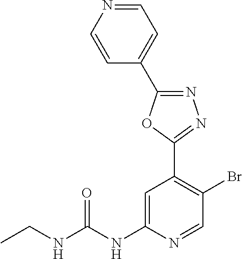

1-(5-Bromo-4-(2-isonicotinoylhydrazinecarbonyl)pyridin-2-yl)-3-ethylurea (Intermediate 52, 538 mg, 1.32 mmol) and triphenyl phosphine (693 mg, 2.64 mmol) were dissolved in methylene chloride (6 mL). Triethylamine (0.369 mL, 2.64 mmol) and carbon tetrabromide (876 mg, 2.64 mmol) were added sequentially. The solution was stirred at room temperature for 12 h, then diluted with water and stirred vigorously for 30 min. The organic and aqueous layers were separated and the organic layer was dried over Na₂SO₄, filtered and concentrated under reduced pressure. The concentrate was dissolved in minimal DMSO and purified by Gilson HPLC. Isolation gave 105 mg of the title compound.

LC/MS (ES⁺)[(M+H)⁺]: 389, 390 for C₁₅H₁₃BrN₆O₂.

### Intermediate 54

### ethyl 6′-(3-ethylureido)-4′-(5-(pyridin-4-yl)-1,3,4-oxadiazol-2-yl)-3,3′-bipyridine-5-carboxylate

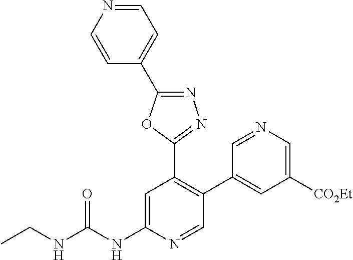

In a microwave reaction vessel, 1-(5-bromo-4-(5-(pyridin-4-yl)-1,3,4-oxadiazol-2-yl)pyridin-2-yl)-3-ethylurea (Intermediate 53, 105 mg, 0.27 mmol), ethyl 5-(4,4,5,5-tetramethyl-1,3,2-dioxaborolan-2-yl)nicotinate (82 mg, 0.30 mmol) and cesium carbonate (34.3 mg, 0.32 mmol) were combined and suspended in a 4:1 mixture of dioxane and water. Pd(PPh₃)₄ (15.59 mg, 0.01 mmol) was added in a single portion. The vessel was sealed, degassed, purged with nitrogen and heated to 100° C. in the microwave for 60 min. The crude reaction mixture was concentrated to dryness. The resulting residue was dissolved in DMSO, filtered and then purified by Gilson HPLC (15-55% ACN/0.1% TFA water in 14 minutes). Isolation gave 58 mg of the title compound.

LC/MS (ES⁺)[(M+H)⁺]: 460 for C₂₃H₂₁N₇O₄.

¹H NMR (300 MHz, d₆-DMSO): 1.05 (t, 3H), 1.23 (t, 3H), 3.16 (m, 2H), 4.29 (q, 2H), 7.38 (m, 1H), 7.63 (d, 2H), 8.30 (t, 1H), 8.37 (s, 1H), 8.45 (s, 1H), 8.76 (d, 2H), 8.83 (d, 1H), 9.08 (d, 1H), 9.53 (s, 1H).

### Intermediate 55

### Butyl 2-butoxy-5-iodonicotinate

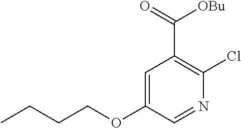

To a solution of methyl-2-chloro-5-iodonicotinate (1.0 g, 3.37 mmol) and butanol (0.74 g, 10 mmol) in THF (80 mL) at 0° C. was added potassium hexamethyldisilizane (20 mL, 0.5N in toluene) drop wise. Slight exotherm was observed. The mixture was stirred at −2-0° C. for 1 h before quenching with acetic acid (0.5 mL) and 6N HCl (0.3 ml). The mixture was diluted with water (80 mL), and extracted with EtOAc (2×150 mL). Combined organic extracts were dried and concentrated. The residue was purified via flash chromatography (10% EtOAc-heptane) to afford 1.2 g (˜90%) of oil as a mixture (˜1:2) of methyl and butyl esters.

MS (ESP): 244.1 (M+H⁺) for C₁₁H₁₄ClNO₃, methyl ester, 286.0 (M+H⁺) for C₁₄H₂₀ClNO₃

### Intermediate 56

### Butoxy-6′-(3-ethylureido)-4′-(4-(trifluoromethyl)thiazol-2-yl)-3,3′-bipyridine-5-carboxylate

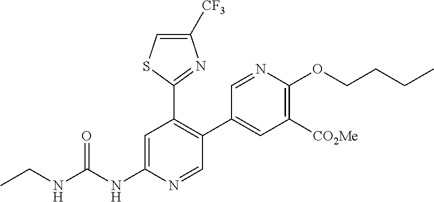

A slurry of 1-ethyl-3-(5-(4,4,5,5-tetramethyl-1,3,2-dioxaborolan-2-yl)-4-(4-(trifluoromethyl)thiazol-2-yl)pyridin-2-yl)urea (Intermediate 12, 1.4 g, 3.18 mmol), 2-butoxy-5-iodonicotinate ester mix (Intermediate 57, 1.2 g, 3.18 mmol) and K₂CO₃ (1.32 g, 9.6 mmol) in 1,4-dioxane-H₂O (25+9 mL) was bubbled with N₂ for 30 min at rt.

Bis(triphenylphosphine)palladium dichloride (0.23 g, 0.32 mmol) was added and the resulting mixture was stirred at 70-80° C. for 1 h. The mixture was cooled to room temperature, diluted with water (50 mL), and the layers were separated. The organic layer was extracted with EtOAc (2×150 mL). Combined organic layers were dried and concentrated. The residue was purified via flash chromatography (10-70% EtOAc-heptane+1% EtOH) to afford 1.6 g (˜70%) of a mixed ester of 6-butoxy-6′-(3-ethylureido)-4′-(4-(trifluoromethyl)thiazol-2-yl)-3,3′-bipyridine-5-carboxylate as light brown gum.

MS (ESP): 524.1 (MH⁺) for C₂₃H₂₄F₃N₅O₄S

¹H NMR (d₆-DMSO): δ 0.91 (t, 3H), 1.10 (t, 3H), 1.39-1.48 (m, 2H), 1.65-1.76 (m, 2H), 3.18 (q, 2H), 3.75 (s, 3H), 4.36 (t, 2H), 7.57 (bt, 1H), 8.02 (d, 1H), 8.20 (s, 1H), 8.29 (m, 2H), 8.53 (s, 1H), 9.44 (s, 1H).

### Intermediate 57

### 6-butoxy-6′-(3-ethylureido)-4′-(4-(trifluoromethyl)thiazol-2-yl)-3,3′-bipyridine-5-carboxylic acid

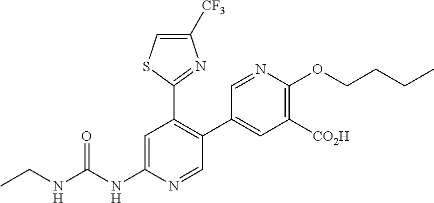

The mixed ester of 6-butoxy-6′-(3-ethylureido)-4′-(4-(trifluoromethyl)thiazol-2-yl)-3,3′-bipyridine-5-carboxylate (Intermediate 56, ˜100 mg) was stirred in 1 N NaOH (˜0.5 mL) in THF (˜3 mL) at 50° C. until no starting materials remained by LCMS. The solvent was evaporated and the sodium salt of product was purified via a reverse-phase column with 5-70% MeOH-water. The product fractions were concentrated and neutralized with HCl (1.0N) to give 6-butoxy-6′-(3-ethylureido)-4′-(4-(trifluoromethyl)thiazol-2-yl)-3,3′-bipyridine-5-carboxylic acid.

MS (ESP): 510.0 (M+H⁺) for C₂₂H₂₂F₃N₅O₄S

¹H NMR (d₆-DMSO): δ 0.93 (t, 3H), 1.10 (t, 3H), 1.39-1.48 (m, 2H), 1.65-1.76 (m, 2H), 3.19 (q, 2H), 4.27 (t, 2H), 7.63 (d, 1H), 7.84 (t, 1H), 7.93 (d, 1H), 8.17 (s, 1H), 8.30 (s, 1H), 8.55 (d, 1H), 9.62 (s, 1H).

### Intermediate 58

### Methyl 2-(bis(tert-butoxycarbonyl)amino)-5-bromoisonicotinate

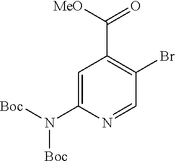

To a 1 L round bottom flask was charged methyl 2-amino-5-bromoisonicotinate (43.7 g, 189 mmol) and tert-butanol (360 mL). The mixture was kept at 30° C., then DMAP (1.40 g, 11.48 mmol, 6%) and di-tert-butyl dicarbonate (105 g, 481 mmol, 2.54 eq) were added. The resulting mixture was heated to 80° C. for 30 minutes, then the reaction mixture was allowed to cool to room temperature and ethanol was added. The precipitated that formed was collected by filtration and washed with ethanol. After drying under vacuum overnight (˜16 hour) at 25° C., the first crop was obtained as 67.8 g of light brown solid (90.2%).

MS (ESP): 277.1 (MH⁺-Boc-tBu) for C₁₇H₂₃BrN₂O₆

¹H NMR (300 MHz, CDCl₃): δ 1.5 (s, 18H), 4.0 (s, 32H), 7.7 (s, 1H), 8.7 (s, 1H).

### Intermediate 59

### tert-butyl 5-bromo-4-carbamoylpyridin-2-ylcarbamate

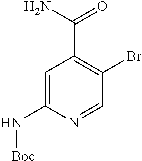

A solution of methyl 2-(bis(tert-butoxycarbonyl)amino)-5-bromoisonicotinate (Intermediate 58, 67.8 g, 157.3 mmol) in 7 N ammonia in methanol (600 mL) was allowed to stir at 40-50° C. in a sealed flask for overnight. The resulting mixture was evaporated to dryness and the crude product was directly used for the next step without further purification.

MS (ESP): 339.9 (M+Na⁺) for C₁₁H₁₄BrN₃O₃

¹H NMR (300 MHz, DMSO-d₆): δ 1.47 (s, 9H), 7.82 (d, 2H), 8.07 (s, 1H), 8.41 (d, 1H), 10.2 (s, 1H).

### Intermediate 60

### tert-butyl 5-bromo-4-carbamothioylpyridin-2-ylcarbamate

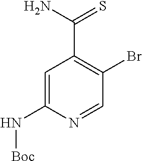

The crude tert-butyl 5-bromo-4-carbamoylpyridin-2-ylcarbamate (Intermediate 59, 157.3 mol) was treated with Lawesson's Reagent (65 g, 157.5 mmol) and tetrahydrofuran (500 mL). The resulting mixture was heated at reflux for 1 h, then it was allowed to stir at room temperature over the weekend. The mixture was concentrated to dryness in vacuo and toluene (˜200 mL) was added. After initiating, a bright yellow solid precipitated, which was collected and washed with toluene, then dried in the vacuum oven at 50° C. for 4 hours, yielding 49 g of a bright yellow solid (94%).

MS (ESP): 354.2 (M+Na⁺) for C₁₁H₁₄BrN₃O₂S

¹H NMR (300 MHz, CDCl₃): δ 1.53 (s, 9H), 7.03 (br, 1H), 7.61 (br, 1H), 7.74 (br, 1H), 8.2 (s, 1H), 8.35 (s, 1H).

### Intermediate 61

### tert-butyl 5-bromo-4-(4-hydroxy-4-(trifluoromethyl)-4,5-dihydrothiazol-2-yl)pyridin-2-ylcarbamate

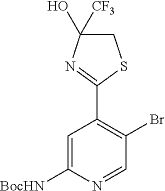

To a 2 L round bottom flask was charged tert-butyl 5-bromo-4-carbamothioylpyridin-2-ylcarbamate (Intermediate 60, 48 g, 145 mmol) in tetrahydrofuran (800 mL), then solid sodium bicarbonate (24.4 g, 290 mmol) was added followed by 1,1,1-trifluoro-3-bromoacetone (31 mL, 290 mmol). The resulting mixture (yellow suspension) was allowed to stir at room temperature overnight. The white suspension was filtered and the solid was washed with water (2.2˜2.5 L). The white solid was dried under vacuum as the 1^{st} crop (54.4, 85% yield). The mother liquor was concentrated to remove the tetrahydrofuran, filtered and washed to give after drying, 3.5 g white solid.

MS (ESP): 386.0 (M-Boc) for C₁₄H₁₅BrF₃N₃O₃S

¹H NMR (300 MHz, CDCl₃): δ 1.6 (s, 9H), 3.3 (br, 2H), 3.6 (d, 1H), 3.9 (d, 1H), 8.2 (s, 1H), 8.5 (s, 1H).

### Intermediate 62

### tert-butyl 5-bromo-4-(4-(trifluoromethyl)thiazol-2-yl)pyridin-2-ylcarbamate

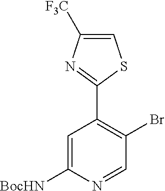

To a 2 L round bottom flask was charged tert-butyl 5-bromo-4-(4-hydroxy-4-(trifluoromethyl)-4,5-dihydrothiazol-2-yl)pyridin-2-ylcarbamate (Intermediate 61, 54.4 g, 123 mmol) and dimethoxyethane (800 mL). The mixture was chilled in an ice-water bath, then trifluoroacetic anhydride (68 mL, 502 mmol) and 2,6-lutidine (128 mL, 1.10 mol) were added simultaneously over 30 min. The temperature of the exothermic reaction was controlled below 6° C. The orange/yellow solution which resulted was allowed to stir in the ice-water bath for half an hour, then was warmed to room temperature for 2 h. The solution was concentrated to dryness, and the residue was triturated with methanol. The precipitated solid was collected and washed with more methanol, and dried under vacuum overnight, yielding 48.3 g of white solid as the 1^{st} crop. The mother liquor was concentrated and triturated with methanol again, the 2^{nd} crop was obtained as a light yellow solid (1.5 g). Totally 49.8 g of product was obtained in 95.4% yield.

MS (ESP): 368.0 (M-Boc) for C₁₄H₁₃BrF₃N₃O₂S

¹H NMR (300 MHz, CDCl₃): δ 1.6 (s, 9H), 8.0 (s, 1H), 8.2 (br, 1H), 8.55 (s, 1H), 8.65 (s, 1H).

### Intermediate 63

### 1-(5-bromo-4-(4-(trifluoromethyl)thiazol-2-yl)pyridin-2-yl)-3-methylurea

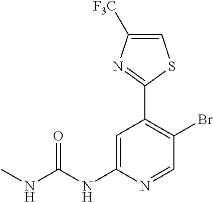

To a sealed tube was charged tert-butyl 5-bromo-4-(4-(trifluoromethyl)thiazol-2-yl)pyridin-2-ylcarbamate (Intermediate 62, 1.2 g) with methylamine (15 mL, 2 M in methanol). The reaction mixture was heated at 145° C. for 2 h in a microwave. The mixture was concentrated to dryness to give desired product as a white solid (quantitative yield). The crude product was used directly for Suzuki couplings without further purification

MS (ESP): 381.0 (MH⁺) for C₁₁H₈BrF₃N₄OS.

### Intermediates 64-69

The following Intermediates were synthesized according to the procedure described for Intermediate 63 from the starting materials listed in the Table.

| Int | Compound | Data | SM |
|---|---|---|---|
| 64 | 1-(5-bromo-4-(4-(trifluoromethyl)thiazol-2- yl)pyridin-2-yl)-3-cyclopropylurea 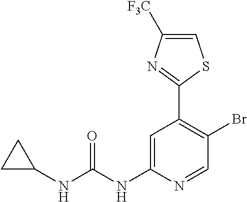 | MS (ESP): 408.9 (MH⁺) C₁₃H₁₀BrF₃N₄OS ¹H NMR (300 MHz, CD₃OD): δ 0.56-0.65 (m, 2H), 0.80-0.85 (m, 2H), 2.65-2.75 (m, 1H), 7.55 (s, 1H), 8.21 (s, 1H), 8.55 (s, 1H) | Intermediate 62 and cyclopropyl amine |
| 65 | 1-(5-bromo-4-(4-(trifluoromethyl)thiazol-2- yl)pyridin-2-yl)-3-cyclohexylurea 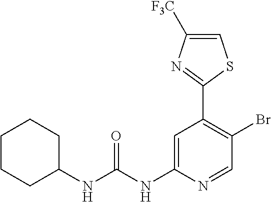 | MS (ESP): 450.9 (MH⁺) for C₁₆H₁₆BrF₃N₄OS | Intermediate 62 and cyclohexyl amine |
| 66 | 3-(5-bromo-4-(4-(trifluoromethyl)thiazol-2- yl)pyridin-2-yl)-1,1-diethylurea 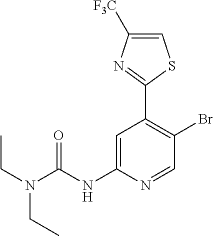 | MS (ESP): 424.9 (MH⁺) for C₁₄H₁₄BrF₃N₄OS | Intermediate 62 and diethylamine |
| 67 | 1-(5-bromo-4-(4-(trifluoromethyl)thiazol-2- yl)pyridin-2-yl)-3-(cyclopropylmethyl)urea 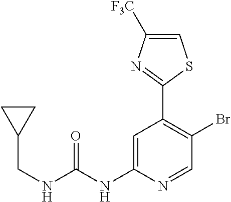 | MS (ESP): 423.0 (MH⁺) for C₁₄H₁₂BrF₃N₄OS ¹H NMR (300 MHz, CD₃OD): δ 0.25-0.30 (m, 2H), 0.55-0.65 (m, 2H), 1.01-1.10 (m, 1H), 3.20 (d, 2H), 7.80 (s, 1H), 8.10 (s, 1H), 8.57 (s, 1H) | Intermediate 62 and cyclopyane- methylamine |
| 68 | 1-(5-bromo-4-(4-(trifluoromethyl)thiazol-2- yl)pyridin-2-yl)-3-(2,2,2-trifluoroethyl)urea 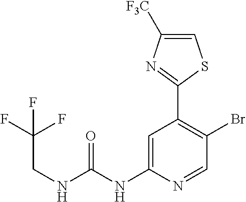 | MS (ESP): 450.9 (MH⁺) for C₁₂H₇BrF₆N₄OS | Intermediate 62 and 1,1,1,- trifluroethylamine |
| 69 | 1-(5-bromo-4-(4-(trifluoromethyl)thiazol-2- yl)pyridin-2-yl)-3-(2,2-difluoroethyl)urea 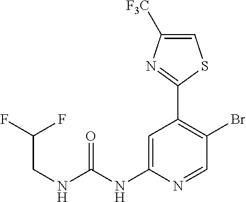 | MS (ESP): 432.9 (MH⁺) for C₁₂H₈BrF₅N₄OS; ¹H NMR (300 MHz, CDCl₃): δ 3.80 (td, 2H), 5.99 (tt, 1H), 7.70 (s, 1H), 8.05 (s, 1H), 8.10 (s, br, 1H), 8.50 (s, 1H), 9.30 (br, 1H) | Intermediate 62 and 1,1-di- fluroethylamine |

### Intermediate 70

### Methyl 6′-(3-methylureido)-4′-(4-(trifluoromethyl)thiazol-2-yl)-3,3′-bipyridine-5-carboxylate

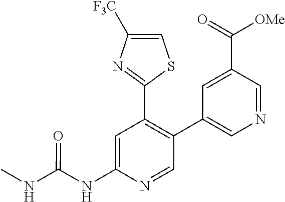

To a sealed tube was charged 1-(5-bromo-4-(4-(trifluoromethyl)thiazol-2-yl)pyridin-2-yl)-3-methylurea (Intermediate 63, 0.41 g, 1.07 mmol), trans dichlorobis(triphenylphosphine)palladium (II) (75 mg, 10 mmol %), 1,4-dioxane (10 mL), sodium bicarbonate (180 mg, 2.14 mmol) in water (10 mL), and then methyl 5-(4,4,5,5-tetramethyl-1,3,2-dioxaborolan-2-yl)nicotinate (0.42 g, 1.61 mmol) was added. The resulting mixture was purged by nitrogen for 5 min then heated at 50° C. for 2 h (in a microwave). Based on LC, the reaction was incomplete and the mixture was further heated at 60° C. 1 h (in a microwave). The resulting mixture was diluted with water, extracted ethyl acetate (3×), and the combined organic layers were dried over sodium sulfate. After concentration, the crude product was purified by Analogix to give the desired product as a white solid (200 mg, 42.7%).

MS (ESP): 438.0 (MH⁺) for C₁₈H₁₄F₃N₅O₃S

¹H NMR (300 MHz, CDCl₃): δ 1.90 (s, 3H), 3.94 (s, 3H), 7.80 (s, 1H), 8.26 (t, 1H), 8.31 (t, 1H), 8.36 (t, 1H), 8.65 (d, 1H), 9.12 (d, 1H)

¹⁹F NMR Spectrum (300 MHz, CD₃OD) −66.05

### Intermediates 71-76

The following Intermediates were prepared according to the procedure described for Intermediate 70 from the starting materials indicated in the Table.

| Int | Compound | Data | SM |
|---|---|---|---|
| 71 | methyl 6′-(3-cyclopropylureido)-4′-(4- (trifluoromethyl)thiazol-2-yl)-3,3′- bipyridine-5-carboxylate 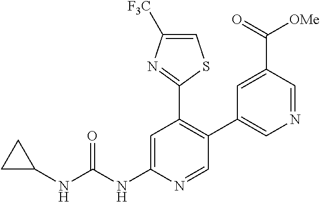 | MS (ESP): 464.1 (MH⁺) for C₂₀H₁₆F₃N₅O₃S ¹H NMR(300 MHz, CD₃OD): δ 0.57-0.60 (m, 2H), 0.77-0.80 (m, 2H), 2.65-2.75 (m, 1H), 3.94 (s, 3H), 7.97 (br, 1H), 8.25 (d, 1H), 8.30 (t, 1H), 8.35 (s, 1H), 8.65 (d, 1H), 9.12 (d, 1H) ¹⁹F NMR (300 MHz, CD₃OD) −66.05 | Intermediate 64 and methyl 5- (4,4,5,5- tetramethyl- 1,3,2- dioxaborolan-2- yl)nicotinate |
| 72 | methyl 6′-(3-cyclohexylureido)-4′-(4- (trifluoromethyl)thiazol-2-yl)-3,3′- bipyridine-5-carboxylate 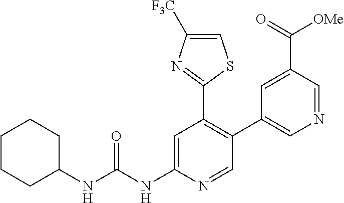 | MS (ESP): 506.1 (MH⁺) for C₂₃H₂₂F₃N₅O₃S ¹H NMR (300 MHz, CD₃OD): δ 1.19-1.46 (m, 5H), 1.2-2.1 (m, 5H), 3.7 (br, 1H), 3.94 (s, 3H), 7.88 (s, 1H), 8.25 (d, 1H), 8.30 (t, 1H), 8.36 (d, 1H), 8.60 (d, 1H), 9.11 (d, 1H) ¹⁹F NMR (300 MHz, CD₃OD) −66.04 | Intermediate 65 and methyl 5- (4,4,5,5- tetramethyl- 1,3,2- dioxaborolan-2- yl)nicotinate |
| 73 | methyl 6′-(3,3-diethylureido)-4′-(4- (trifluoromethyl)thiazol-2-yl)-3,3′- bipyridine-5-carboxylate 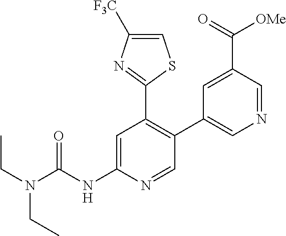 | MS (ESP): 480.0 (MH⁺) for C₂₁H₂₀F₃N₅O₃S ¹H NMR (300 MHz, CD₃OD): δ 1.25 (t, 6H), 3.49 (q, 4H), 3.94 (s, 3H), 8.25 (d, 1H), 8.31 (t, 1H), 8.39 (d, 1H), 8.53 (d, 2H), 8.65 (d, 1H), 9.11 (d, 1H) ¹⁹F NMR (300 MHz, CD₃OD) −66.04 | Intermediate 66 and methyl 5- (4,4,5,5- tetramethyl- 1,3,2- dioxaborolan-2- yl)nicotinate |
| 74 | methyl 6′-(3-cyclopropylmethyl)ureido)-4′- (4-(trifluoromethyl)thiazol-2-yl)-3,3′- bipyridine-5-carboxylate 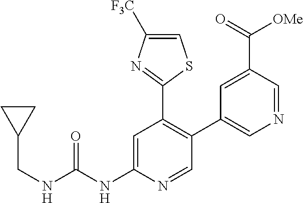 | MS (ESP): 478.2 (MH⁺) for C₂₁H₁₈F₃N₅O₃S; ¹H NMR (300 MHz, CD₃OD): δ 0.27-0.31 (m, 2H), 0.51-0.58 (m, 2H), 1.07-1.20 (m, 1H), 3.20 (d, 2H), 3.95 (s, 3H), 7.88 (s, 1H), 8.25 (d, 1H), 8.31 (t, 1H), 8.37 (s, 1H), 8.66 (d, 1H), 9.12 (d, 1H); ¹⁹F NMR (300 MHz, CD₃OD) −66.16 | Intermediate 67 and methyl 5- (4,4,5,5- tetramethyl- 1,3,2- dioxaborolan-2- yl)nicotinate |
| 75 | methyl 6′-(3-(2,2,2-trifluoroethyl)ureido)-4′- (4-(trifluoromethyl)thiazol-2-yl)-3,3′- bipyridine-5-carboxylate 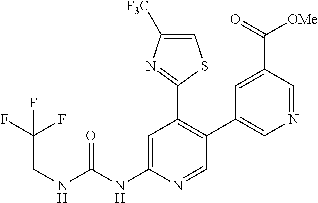 | MS (ESP): 506.1 (MH⁺) for C₁₉H₁₃F₆N₅O₃S; ¹H NMR (300 MHz, CD₃OD): δ 3.95 (s, 3H), 4.06 (q, 2H), 7.91 (s, 1H), 8.26 (d, 1H), 8.32 (t, 1H), 8.39 (d, 1H), 8.66 (d, 1H), 9.13 (d, 1H); ¹⁹F NMR (300 MHz, CD₃OD) −66.04 (s, 3F), −74.95 (t, 3F) | Intermediate 68 and methyl 5- (4,4,5,5- tetramethyl- 1,3,2- dioxaborolan-2- yl)nicotinate |
| 76 | methyl 6′-(3-(2,2-difluoroethyl)ureido)-4′- (4-(trifluoromethyl)thiazol-2-yl)-3,3′- bipyridine-5-carboxylate 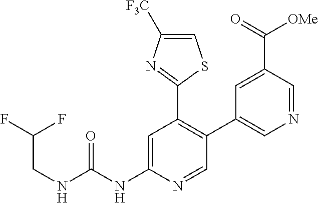 | MS (ESP): 488.1 (MH⁺) for C₁₉H₁₄F₅N₅O₃S; ¹H NMR (300 MHz, CD₃OD): δ 3.71 (td, 2H), 3.94 (s, 3H), 5.99 (tt, 1H), 7.88 (s, 1H), 8.25 (d, 1H), 8.31 (t, 1H), 8.37 (d, 1H), 8.66 (d, 1H), 9.12 (d, 1H); ¹⁹F NMR (300 MHz, CD₃OD) −66.04 (s, 3F), −125.33 (t, 1F), −125.53 (t, 1F) | Intermediate 69 and methyl 5- (4,4,5,5- tetramethyl- 1,3,2- dioxaborolan-2- yl)nicotinate |

### Intermediate 77

### Methyl 6′-(tert-butoxycarbonylamino)-4′-(4-(trifluoromethyl)thiazol-2-yl)-3,3′-bipyridine-5-carboxylate

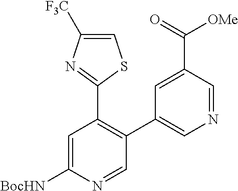

A solution of potassium carbonate (4 g, 28.9 mmol) in water (250 mL) was prepared and purged by N₂ for a few minutes. To a 1 L round bottom flask was charged tert-butyl 5-bromo-4-(4-(trifluoromethyl)thiazol-2-yl)pyridin-2-ylcarbamate (Intermediate 62, 6 g, 14.2 mmol), trans dichlorobis(triphenylphosphine)palladium (II) (997 mg, 10 mol %) and 1,4-dioxane (300 mL). The prepared potassium carbonate solution was added followed by methyl 5-(4,4,5,5-tetramethyl-1,3,2-dioxaborolan-2-yl)nicotinate (5.58 g, 21.2 mmol) and the mixture further diluted with 1,4-dioxane (200 mL). The resulting brown solution was purged by N₂ for a further ˜10-15 min then heated to 55° C. (reflux) for ˜10-15 min. The brown solution became black. After 1 h the reaction went to completion based on LCMS. The mixture was allowed to cool and was diluted by ethyl acetate (200 mL), then washed with brine twice. The combined aqueous layers were backwashed with ethyl acetate (400 mL), and the combined organic layers dried over sodium sulfate. After concentration, a gray solid was obtained. The crude solid was purified by a silica gel plug eluted with ethyl acetate/heptane (3:4 or 3:5). After concentration, the resulting solid was further triturated with ethanol to give 5.6 g white fluffy solid. The mother liquor was concentrated and triturated with ethanol to give a 2^{nd} crop as a white solid (0.33 g). Totally 5.93 g of product was obtained (87.2%).

MS (ESP): 481.2 (MH⁺) for C₂₁H₁₉F₃N₄O₄S

¹H NMR (300 MHz, CDCl₃): δ 1.60 (s, 9H), 3.9 (s, 3H), 7.6 (s, 1H), 7.8 (d, 1H), 8.30 (t, 1H), 8.35 (s, 1H), 8.5 (s, 1H), 8.6 (d, 1H), 9.2 (d, 1H).

### Intermediate 78

### methyl 6′-amino-4′-(4-(trifluoromethyl)thiazol-2-yl)-3,3′-bipyridine-5-carboxylate

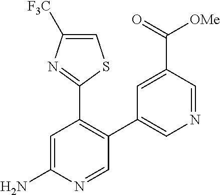

To a 250 mL round bottom flask was charged methyl 6′-(tert-butoxycarbonylamino)-4′-(4-(trifluoromethyl)thiazol-2-yl)-3,3′-bipyridine-5-carboxylate (Intermediate 77, 1.6 g, 3.33 mmol) with 4 M HCl in 1,4-dioxane (110 mL), and the resulting clear solution was stirred at room temperature over the weekend (two days). Saturated sodium bicarbonate was added to the suspension was to neutralize the acid. The resulting clear solution was extracted with ethyl acetate (3×), and the combined organic layers were dried over sodium sulfate. After concentration and drying, a yellow fluffy solid was obtained in quantitative yield which was used without purification.

MS (ESP): 381.0 (MH⁺ average) for C₁₁H₈BrF₃N₄OS

### Intermediate 79

### methyl 6′-(3-isopropylureido)-4′-(4-(trifluoromethyl)thiazol-2-yl)-3,3′-bipyridine-5-carboxylate

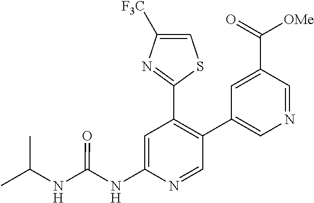

To a sealed tube was charged methyl 6′-amino-4′-(4-(trifluoromethyl)thiazol-2-yl)-3,3′-bipyridine-5-carboxylate (Intermediate 78, 330 mg, 0.87 mmol) with chloroform (5 mL), then isopropyl isocyanate (0.5 mL) was added. The resulting mixture was heated at 50° C. (oil bath) for 24 hours. The reaction was incomplete. More isopropyl isocyanate (1 mL) was added and the mixture heated at 50° C. (oil bath) for another 3 days. The resulting suspension was concentrated to dryness and triturated by ethanol. After filtration and drying, a white solid was obtained (300 mg, 74.3%).

MS (ESP): 466.2 (MH⁺) for C₂₀H₁₈F₃N₅O₃S

¹H NMR (300 MHz, CD₃OD): δ 1.24 (d, 6H), 3.93-4.02 (m, 1H), 3.93 (s, 3H), 7.88 (s, 1H), 8.24 (s, 1H), 8.30 (t, 1H), 8.35 (s, 1H), 8.64 (d, 1H), 9.11 (d, 1H)

¹⁹F NMR (300 MHz, CD₃OD) −66.00

### Intermediate 80

### methyl 6′-(3-propylureido)-4′-(4-(trifluoromethyl)thiazol-2-yl)-3,3′-bipyridine-5-carboxylate

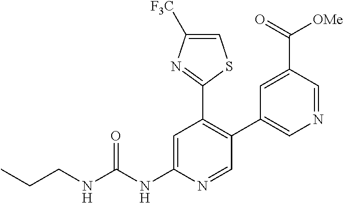

To a sealed tube was charged methyl 6′-amino-4′-(4-(trifluoromethyl)thiazol-2-yl)-3,3′-bipyridine-5-carboxylate (Intermediate 78, 350 mg, 0.921 mmol) with chloroform (5 mL), then propyl isocyanate (1.75 mL) was added. The resulting mixture was heated at 50° C. (oil bath) for 4 days. The resulting suspension was concentrated to dryness and triturated by methanol. After filtration and drying, a white solid was obtained (257 mg, 60%).

MS (ESP): 466.2 (MH⁺) for C₂₀H₁₈F₃N₅O₃S

¹H NMR (300 MHz, CD₃OD): δ 0.99 (t, 3H), 1.58-1.66 (m, 2H), 3.29 (t, 2H), 3.94 (s, 3H), 7.84 (s, 1H), 8.24 (s, 1H), 8.30 (t, 1H), 8.35 (s, 1H), 8.64 (d, 1H), 9.11 (d, 1H)

¹⁹F NMR (300 MHz, CD₃OD) −66.00

### Intermediate 81

### ethyl 2-(5-bromo-2-(3-ethylureido)pyridin-4-yl)-4-(trifluoromethyl)thiazole-5-carboxylate

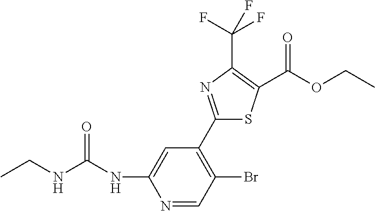

A suspension of 5-bromo-2-(3-ethylureido)pyridine-4-carbothioamide (Intermediates, 5.0 g, 16.5 mmol), ethyl 2-chloro-3-keto-4,4,4-trifluorobutyrate (25 g, 114 mmol) in acetonitrile (250 mL) was heated at 60° C. for 6 days. The solution was cooled triethylamine (12 mL, 87 mmol) was added followed by the dropwise addition of methane sulfonyl chloride (3.0 mL, 39 mmol). This mixture was then stirred at room temperature overnight. The solid was filtered, washed with water (500 mL) and dried in the vacuum oven at 50° C. for 12 hours to give 3.2 g (41%) of ethyl 2-(5-bromo-2-(3-ethylureido)pyridin-4-yl)-4-(trifluoromethyl)thiazole-5-carboxylate as a pale yellow solid.

MS (ESP): 467.1 and 468.9 (M+H⁺) for C₁₅H₁₄BrF₃N₄O₃S

¹H NMR (300 MHz, DMSO-d₆): δ 1.08 (t, 3H), 1.34 (t, 3H), 3.17 (m, 2H), 4.40 (q, 2H), 7.20 (t, 1H), 8.54 (s, 1H), 8.59 (s, 1H), 9.43 (bs, 1H)

### Intermediate 82

### 1-(5-bromo-4-(5-(hydroxymethyl)-4-(trifluoromethyl)thiazol-2-yl)pyridin-2-yl)-3-ethylurea

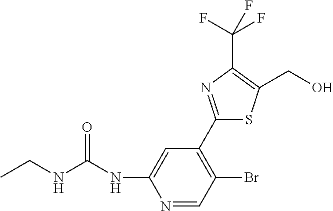

To a suspension of ethyl 2-(5-bromo-2-(3-ethylureido)pyridin-4-yl)-4-(trifluoromethyl)thiazole-5-carboxylate (Intermediate 81, 4.7 g, 10 mmol) in tetrahydrofuran (85 mL), was added lithium borohydride powder (653 mg, 30 mmol). The reaction was stirred for 3 hours at room temperature during which time the solution turned yellow and homogeneous. Water (50 mL) was then carefully added to the reaction and the organics were removed in vacuo. The remaining aqueous phase was extracted with ethyl acetate (3×, 50 mL). The organic extracts were combined, dried over sodium sulfate, and the solvent was removed in vacuo. This gave 4.2 g (92%) of 1-(5-bromo-4-(5-(hydroxymethyl)-4-(trifluoromethyl)thiazol-2-yl)pyridin-2-yl)-3-ethylurea as a pale yellow solid.

MS (ESP): 424.8 and 426.9 (M+H⁺) for C₁₃H₁₂BrF₃N₄O₂S

¹H NMR (300 MHz, DMSO-d₆): δ 1.08 (t, 3H), 3.17 (m, 2H), 4.93 (m, 2H), 6.37 (bt, 1H), 7.26 (bt, 1H), 8.38 (s, 1H), 8.54 (s, 1H), 9.38 (bs, 1H).

### Intermediates 83-85

The following Intermediates were synthesized by the general procedure described below from the starting material indicated in the Table.

### General Procedure

Ethyl 2-(5-bromo-2-(3-ethylureido)pyridin-4-yl)-4-(trifluoromethyl)thiazole-5-carboxylate (Intermediate 81, 0.5 g) and excess amine (neat or 4-6 eq in ethanol solution) was heated to 80-90° C. in microwave for 3 h. The solid that formed were collected by filtration and washed by methyl tert-butyl ether to give the desired product as pale yellow or off-white solid.

| Int | Compound | Data | SM |
|---|---|---|---|
| 83 | 2-(5-bromo-2-(3-ethylureido)pyridin-4-yl)- N-(2-methoxyethyl)-4- (trifluoromethyl)thiazole-5-carboxamide 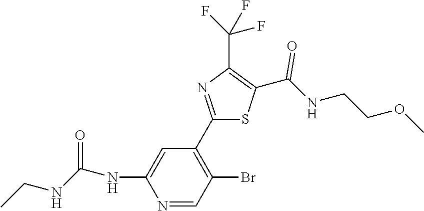 | MS (ESP): 495.9 and 498.0 (M + H⁺) for C₁₆H₁₇BrF₃N₅O₃S; ¹H NMR (300 MHz, DMSO-d₆): δ 1.08 (t, 3H), 3.17 (m, 2H), 3.28 (s, 3H), 3.45 (m, 4H), 7.22 (t, 1H), 8.46 (s, 1H), 8.59 (s, 1H), 9.18 (t, 1H), .43 (bs, 1H) | Intermediate 81 and methoxyethyl- amine |
| 84 | 2-(5-bromo-2-(3-ethylureido)pyridin-4-yl)- N-(2-morpholinoethyl)-4- (trifluoromethyl)thiazole-5-carboxamide 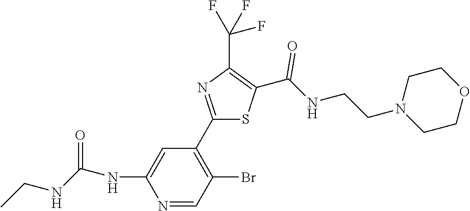 | MS (ESP): 551.0 and 552.9 (M + H⁺) for C₁₉H₂₂BrF₃N₆O₃S; ¹H NMR (300 MHz, DMSO-d₆): δ 1.08 (t, 3H), 2.41 (m, 6H), 3.18 (m, 2H), 3.38 (m, 2H), 3.57 (m, 4H), 7.21 (t, 1H), 8.45 (s, 1H), 8.58 (s, 1H), 9.02 (t, 1H), .42 (bs, 1H) | Intermediate 81 and 2- morpholino- ethanamine |
| 85 | 2-(5-bromo-2-(3-ethylureido)pyridin-4-yl)- N-(2-(4-methylpiperazin-1-yl)ethyl)-4- (trifluoromethyl)thiazole-5-carboxamide 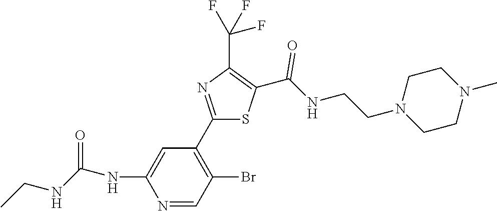 | MS (ESP): 564.0 and 566.1 (M + H⁺) for C₂₀H₂₅BrF₃N₇O₂S ¹H NMR (300 MHz, DMSO-d₆): δ 1.08 (t, 3H), 2.14 (s, 3H), 2.44 (m, 10H), 3.18 (m, 2H), 3.36 (m, 2H), 7.21 (t, 1H), 8.45 (s, 1H), 8.58 (s, 1H), 8.98 (t, 1H), .43 (bs, 1H) | Intermediate 81 and 2-(4-methyl- piperazin-1- yl)ethanamine |

### Intermediates 86-89

The following Intermediates were synthesized by the general procedure described below from the starting material indicated in the Table.

### General Procedure

Ethyl 2-(5-bromo-2-(3-ethylureido)pyridin-4-yl)-4-(trifluoromethyl)thiazole-5-carboxylate (Intermediate 81, 0.5 g), magnesium chloride (1 eq) and excess amine (4-6 eq in ethanol solution) was heated to 90° C. in microwave for 3 h. The solid was filtered, washed by water and methyl tert-butyl ether, then dried in vacuum oven at 50° C. for 12 hrs to give desired product as pale yellow or off-white solid.

| Int | Compound | Data | SM |
|---|---|---|---|
| 86 | 2-(5-bromo-2-(3-ethylureido)pyridin-4-yl)-N-(tetrahydro-2H- pyran-4-yl)-4-(trifluoromethyl)thiazole-5-carboxamide 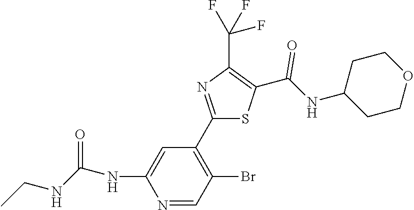 | MS (ESP): 522.0 and 524.1 (M + H⁺) for C₁₈H₁₉BrF₃N₅O₃S ¹H NMR (300 MHz, DMSO-d₆): δ 1.08 (t, 3H), 1.48 (m, 2H), 1.80 (m, 2H), 3.17 (m, 2H), 3.40 (m, 2H), 3.87 (m, 2H), 3.98 (m, 1H), 7.21 (t, 1H), 8.45 (s, 1H), 8.58 (s, 1H), 9.10 (d, 1H), .42 (bs, 1H) | Intermediate 81 and tetrahydro- 2H-pyran-4- amine |
| 87 | 2-(5-bromo-2-(3-ethylureido)pyridin-4-yl)-N-cyclohexyl-4- (trifluoromethyl)thiazole-5-carboxamide 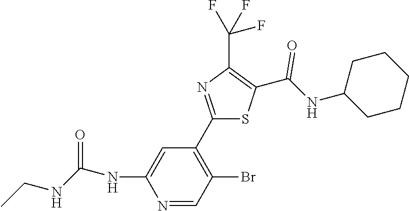 | MS (ESP): 520.2 and 522.0 (M + H⁺) for C₁₉H₂₁BrF₃N₅O₂S ¹H NMR (300 MHz, DMSO-d₆): δ 1.08 (t, 3H), 1.26 (m, 5H), 1.69 (m, 5H), 3.17 (m, 2H), 3.72 (m, 1H), 7.21 (t, 1H), 8.44 (s, 1H), 8.58 (s, 1H), 8.97 (d, 1H), .41 (bs, 1H) | Intermediate 81 and cyclohexanamine |
| 88 | 2-(5-bromo-2-(3-ethylureido)pyridin-4-yl)-N-cyclopentyl-4- (trifluoromethyl)thiazole-5-carboxamide 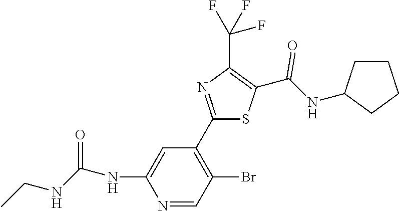 | MS (ESP): 506.1 and 507.9 (M + H⁺) for C₁₈H₁₉BrF₃N₅O₂S ¹H NMR (300 MHz, DMSO-d₆): δ 1.08 (t, 3H), 1.66 (m, 6H), 1.89 (m, 2H), 3.18 (m, 2H), 4.18 (m, 1H), 7.22 (t, 1H), 8.44 (s, 1H), 8.58 (s, 1H), 9.04 (d, 1H), .42 (bs, 1H) | Intermediate 81 and cyclopentanamine |
| 89 | 2-(5-bromo-2-(3-ethylureido)pyridin-4-yl)-N-cyclopropyl-4- (trifluoromethyl)thiazole-5-carboxamide 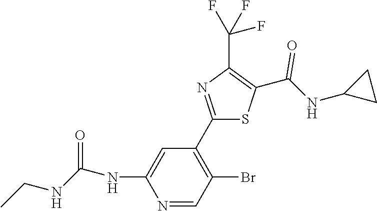 | MS (ESP): 477.9 and 480.0 (M + H⁺) for C₁₆H₁₅BrF₃N₅O₂S ¹H NMR (300 MHz, DMSO-d₆): δ 0.55 (m, 2H), 0.74 (m, 2H), 1.08 (t, 3H), 2.84 (m, 1H), 3.17 (m, 2H), 7.24 (t, 1H), 8.45 (s, 1H), 8.58 (s, 1H), 9.14 (d, 1H), .44 (bs, 1H) | Intermediate 81 and cyclopropanamine |

### Intermediates 90-96

The following Intermediates were synthesized by the general procedure described below from the starting material indicated in the Table.

### General Procedure

1-(5-Bromo-4-(5-(hydroxymethyl)-4-(trifluoromethyl)thiazol-2-yl)pyridin-2-yl)-3-ethylurea (Intermediate 82, 0.5 g, 1.17 mmol) was dissolved in tetrahydrofuran (15 mL). Triethylamine (448 μl, 3.5 mmol) and methane sulfonyl chloride (137 μl, 1.77 mmol) were added sequentially and the reaction was stirred for 2 hours. The appropriate amine (5.9 mmol) was added, and the reaction stirred for an additional 18 hours at room temperature. The solvent was then removed in vacuo and saturated sodium bicarbonate (3 mL) was added. The suspension was extracted with ethyl acetate (3×, 3 mL) and the organic phases were combined, dried over sodium sulfate, and the solvent was removed in vacuo. The products were used without further purification.

| Int | Compound | Data | SM |
|---|---|---|---|
| 90 | 1-(5-bromo-4-(5-((2- methoxyethylamino)methyl)-4- (trifluoromethyl)thiazol-2-yl)pyridin-2-yl)- 3-ethylurea 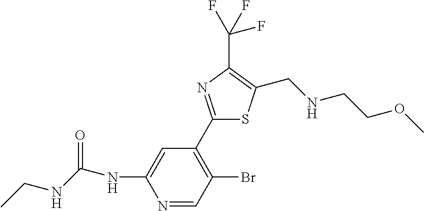 | MS (ESP): 482.1, 484.1 (M + H⁺) for C₁₆H₁₉BrF₃N₅O₂S ¹H NMR (300 MHz, DMSO-d₆): δ 1.11 (t, 3H), 2.78 (t, 2H), 3.19 (m, 2H), 3.36 (s, 3H), 3.41 (t, 2H), 4.16 (s, 2H), 7.26 (t, 1H), 8.31 (s, 1H), 8.56 (s, 1H), 9.38 (bs, 1H). | Intermediate 82 and methoxy- ethylamine |
| 91 | 1-(5-bromo-4-(5-((2- morpholinoethylamino)methyl)-4- (trifluoromethyl)thiazol-2-yl)pyridin-2-yl)- 3-ethylurea 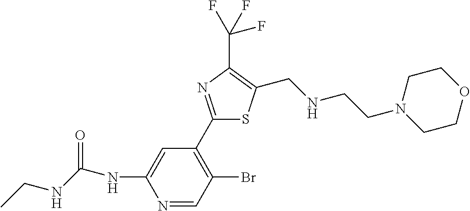 | MS (ESP): 537.0, 539.1 (M + H⁺) for C₁₉H₂₄BrF₃N₆O₂S; ¹H NMR (300 MHz, DMSO-d₆): δ 1.11 (t, 3H), 2.28-2.44 (m, 6H), 2.79 (t, 2H), 3.58 (m, 4H), 4.14 (s, 2H), 7.21 (t, 1H), 8.31 (s, 1H), 8.52 (s, 1H), 9.36 (bs, 1H). | Intermediate 82 and 2-morpholino- ethanamine |
| 92 | 1-(5-bromo-4-(5-((2-(4-methylpiperazin-1- yl)ethylamino)methyl)-4- (trifluoromethyl)thiazol-2-yl)pyridin-2-yl)- 3-ethylurea 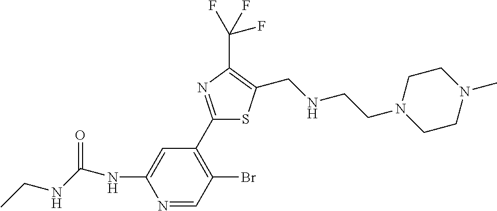 | MS (ESP): 550.2, 552.2 (M + H⁺) for C₂₀H₂₇BrF₃N₇OS; ¹H NMR (300 MHz, DMSO-d₆): δ 1.11 (t, 3H), 2.16 (s, 3H), 2.21- 2.44 (m, 10H), 2.76 (t, 2H), 3.18 (m, 2H), 4.14 (s, 2H), 7.22 (t, 1H), 8.33 (s, 1H), 8.57 (s, 1H), 9.39 (bs, 1H). | Intermediate 82 and 2-(4-methyl- piperazin-1- yl)ethanamine |
| 93 | 1-(5-bromo-4-(5- ((cyclopropylamino)methyl)-4- (trifluoromethyl)thiazol-2-yl)pyridin-2-yl)- 3-ethylurea 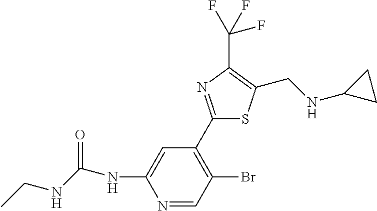 | MS (ESP): 464.1, 465.9 (M + H⁺) for C₁₆H₁₇BrF₃N₅OS; ¹H NMR (300 MHz, DMSO-d₆): δ 0.24 (m, 2H), 0.41 (m, 2H), 1.11 (t, 3H), 2.21 (m, 1H), 3.17 (m, 2H), 4.18 (s, 2H), 7.22 (t, 1H), 8.33 (s, 1H), 8.54 (s, 1H), 9.37 (bs, 1H). | Intermediate 82 and cyclo- propanamine |
| 94 | 1-(5-bromo-4-(5- ((cyclohexylamino)methyl)-4- (trifluoromethyl)thiazol-2-yl)pyridin-2-yl)- 3-ethylurea 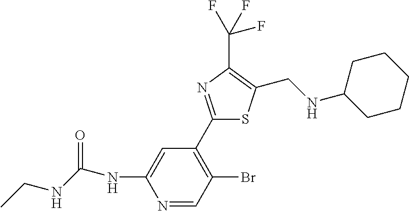 | MS (ESP): 506.1, 507.9 (M + H⁺) for C₁₉H₂₃BrF₃N₅OS; ¹H NMR (300 MHz, DMSO-d₆): δ 1.03-1.38 (m, 6H), 1.11 (t, 3H), 1.65 (m, 2H), 1.82 (m, 2H), 2.86 (m, 1H), 3.18 (m, 2H), 4.13 (s, 2H), 7.23 (t, 1H), 8.32 (s, 1H), 8.56 (s, 1H), 9.40 (bs, 1H). | Intermediate 82 and cyclohexanamine |
| 95 | 1-(5-bromo-4-(5- ((cyclopentylamino)methyl)-4- (trifluoromethyl)thiazol-2-yl)pyridin-2-yl)- 3-ethylurea 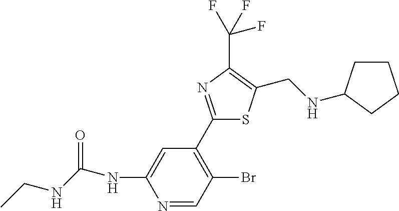 | MS (ESP): 553.2 (M + H⁺) for C₁₈H₂₁BrF₃N₅OS; ¹H NMR (300 MHz, DMSO-d₆): δ 1.11 (t, 3H), 1.36 (m, 2H), 1.44 (m, 2H), 1.63 (m, 2H), 1.71 (m, 2H), 2.93 (m, 1H), 3.19 (m, 2H), 4.08 (s, 2H), 7.21 (t, 1H), 8.33 (s, 1H),8.54 (s, 1H), 9.38 (bs, 1H). | Intermediate 82 and cyclopentanamine |
| 96 | 1-(5-bromo-4-(5-((tetrahydro-2H-pyran-4- ylamino)methyl)-4-(trifluoromethyl)thiazol- 2-yl)pyridin-2-yl)-3-ethylurea 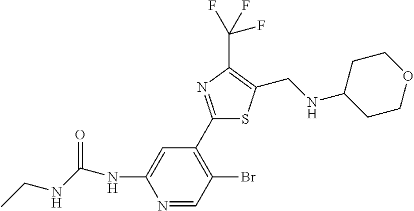 | MS (ESP): 507.9, 510.0 (M + H⁺) for C₁₈H₂₁BrF₃N₅O₂S; ¹H NMR (300 MHz, DMSO-d₆): δ 1.11 (t, 3H), 1.29 (m, 2H), 1.80 (m, 2H), 3.01 (m, 1H), 3.19 (m, 2H), 3.28 (m, 2H), 3.81 (m, 2H), 4.16 (s, 2H), 7.22 (t, 1H), 8.36 (s, 1H), 8.52 (s, 1H), 9.38 (bs, 1H). | Intermediate 82 and tetrahydro- 2H-pyran-4- amine |

### Intermediate 97

### Methyl 6′-(3-ethylureido)-4′-(5-((2-methoxyethylamino)methyl)-4-(trifluoromethyl)thiazol-2-yl)-3,3′-bipyridine-5-carboxylate

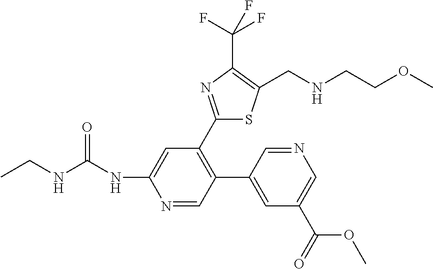

1-(5-bromo-4-(5-((2-methoxyethylamino)methyl)-4-(trifluoromethyl)thiazol-2-yl)pyridin-2-yl)-3-ethylurea (Intermediate 90, ˜500 mg, 1 mmol), methyl 5-(4,4,5,5-tetramethyl-1,3,2-dioxaborolan-2-yl)nicotinate (0.40 g, 1.5 mmol), and trans dichlorobis(triphenylphosphine)palladium (II) (70 mg, 0.1 mmol) were dissolved in 1,4-dioxane (10 mL). Sodium bicarbonate (252 mg, 3 mmol) was dissolved in water (3 mL) and added to the above mixture. The reaction was heated at 110° C. in a microwave for 30 minutes. Ethyl acetate (10 mL) was then added to the reaction and the layers were separated. The solvent was removed in vacuo and the residue was chromatographed on a 12 g Analogix column using 0-10% methanol in dichloromethane. The product containing fractions were combined to give the product ester (65% yield).

MS (ESP): 539.1 (M+H⁺) for C₂₃H₂₅F₃N₆O₄S

¹H NMR (300 MHz, DMSO-d₆): δ 1.11 (t, 3H), 3.21 (m, 2H), 3.23 (s, 3H), 3.37 (m, 4H), 3.89 (s, 3H), 7.49 (t, 1H), 8.24 (1H), 8.28 (t, 1H), 8.37 (s, 1H), 8.74 (d, 1H), 9.02 (t, 1H), 9.11 (d, 1H), 9.52 (bs, 1H).

### Intermediates 98-103

The following Intermediates were prepared as described for Intermediate 97 from the starting materials indicated in the Table.

| Int | Compound | Data | SM |
|---|---|---|---|
| 98 | Methyl 6′-(3-ethylureido)-4′-(5-((2 morpholinoethylamino)methyl)-4- (trifluoromethyl)thiazol-2-yl)-3,3′- bipyridine-5-carboxylate 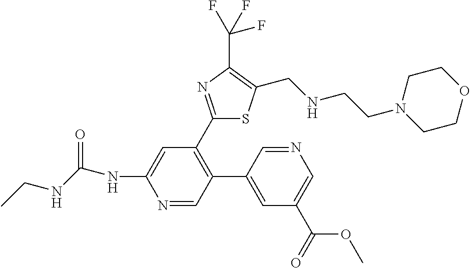 | MS (ESP): 594.0 (M + H⁺) for C₂₆H₃₀F₃N₇O₄S; ¹H NMR (300 MHz, DMSO-d₆): δ 1.11 (t, 3H), 3.21 (m, 2H), 3.23 (s, 3H), 3.37 (m, 4H), 3.89 (s, 3H), 7.49 (t, 1H), 8.24 (1H), 8.28 (t, 1H), 8.37 (s, 1H), 8.74 (d, 1H), 9.02 (t, 1H), 9.11 (d, 1H), 9.52 (bs, 1H). | Intermediate 91 and methyl 5- (4,4,5,5- tetramethyl- 1,3,2- dioxaborolan-2- yl)nicotinate |
| 99 | Methyl 6′-(3-ethylureido)-4′-(5-((2-(4- methylpiperazin-1-yl)ethylamino)methyl)- 4-(trifluoromethyl)thiazol-2-yl)-3,3′- bipyridine-5-carboxylate 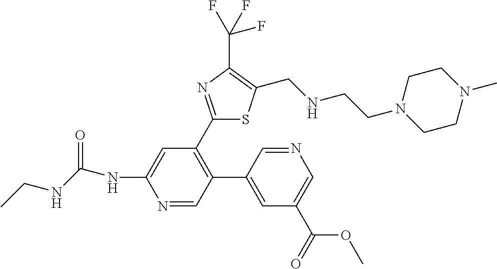 | MS (ESP): 521.2 (M + H⁺) for C₂₃H₂₃F₃N₆O₃S; ¹H NMR (300 MHz, DMSO-d₆): δ 1.11 (t, 3H), 3.21 (m, 2H), 3.23 (s, 3H), 3.37 (m, 4H), 3.89 (s, 3H), 7.49 (t, 1H), 8.24 (1H), 8.28 (t, 1H), 8.37 (s, 1H), 8.74 (d, 1H), 9.02 (t, 1H), 9.11 (d, 1H), 9.52 (bs, 1H). | Intermediate 92 and methyl 5- (4,4,5,5- tetramethyl- 1,3,2- dioxaborolan-2- yl)nicotinate |
| 100 | Methyl 6′-(3-ethylureido)-4′-(5- ((cyclopropylamino)methyl)-4- (trifluoromethyl)thiazol-2-yl)-3,3′- bipyridine-5-carboxylate 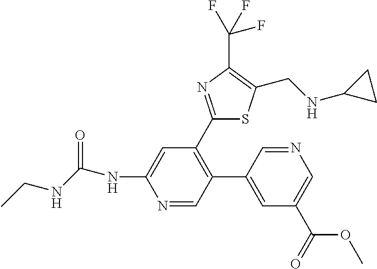 | MS (ESP): 521.2 (M + H⁺) for C₂₃H₂₃F₃N₆O₃S; ¹H NMR (300 MHz, DMSO-d₆): δ 1.11 (t, 3H), 3.21 (m, 2H), 3.23 (s, 3H), 3.37 (m, 4H), 3.89 (s, 3H), 7.49 (t, 1H), 8.24 (1H), 8.28 (t, 1H), 8.37 (s, 1H), 8.74 (d, 1H), 9.02 (t, 1H), 9.11 (d, 1H), 9.52 (bs, 1H). | Intermediate 93 and methyl 5- (4,4,5,5- tetramethyl- 1,3,2- dioxaborolan-2- yl)nicotinate |
| 101 | Methyl 6′-(3-ethylureido)-4′-(5- ((cyclohexylamino)methyl)-4- (trifluoromethyl)thiazol-2-yl)-3,3′- bipyridine-5-carboxylate 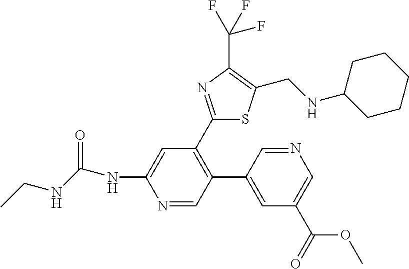 | MS (ESP): 563.1 (M + H⁺) for C₂₆H₂₉F₃N₆O₃S; ¹H NMR (300 MHz, DMSO-d₆): δ 1.11 (t, 3H), 3.21 (m, 2H), 3.23 (s, 3H), 3.37 (m, 4H), 3.89 (s, 3H), 7.49 (t, 1H), 8.24 (1H), 8.28 (t, 1H), 8.37 (s, 1H), 8.74 (d, 1H), 9.02 (t, 1H), 9.11 (d, 1H), 9.52 (bs, 1H). | Intermediate 94 and methyl 5- (4,4,5,5- tetramethyl- 1,3,2- dioxaborolan-2- yl)nicotinate |
| 102 | Methyl 6′-(3-ethylureido)-4′-(5- ((cyclopentylamino)methyl)-4- (trifluoromethyl)thiazol-2-yl)-3,3′- bipyridine-5-carboxylate 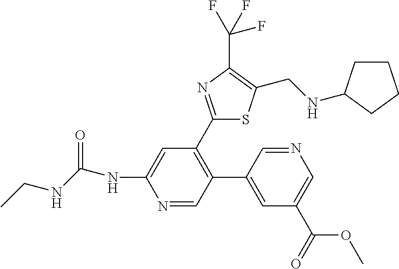 | MS (ESP): 549.0 (M + H⁺) for C₂₅H₂₇F₃N₆O₃S; ¹H NMR (300 MHz, DMSO-d₆): δ 1.11 (t, 3H), 3.21 (m, 2H), 3.23 (s, 3H), 3.37 (m, 4H), 3.89 (s, 3H), 7.49 (t, 1H), 8.24 (1H), 8.28 (t, 1H), 8.37 (s, 1H), 8.74 (d, 1H), 9.02 (t, 1H), 9.11 (d, 1H), 9.52 (bs, 1H). | Intermediate 95 and methyl 5- (4,4,5,5- tetramethyl- 1,3,2- dioxaborolan-2- yl)nicotinate |
| 103 | Methyl 6′-(3-ethylureido)-4′-(5- ((tetrahydro-2H-pyran-4-ylamino)methyl)- 4-(trifluoromethyl)thiazol-2-yl)-3,3′- bipyridine-5-carboxylate 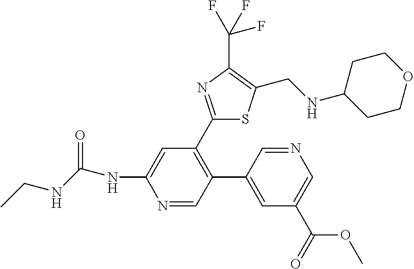 | MS (ESP): 565.2 (M + H⁺) for C₂₅H₂₇F₃N₆O₄S; ¹H NMR (300 MHz, DMSO-d₆): δ 1.11 (t, 3H), 3.21 (m, 2H), 3.23 (s, 3H), 3.37 (m, 4H), 3.89 (s, 3H), 7.49 (t, 1H), 8.24 (1H), 8.28 (t, 1H), 8.37 (s, 1H), 8.74 (d, 1H), 9.02 (t, 1H), 9.11 (d, 1H), 9.52 (bs, 1H). | Intermediate 96 and methyl 5- (4,4,5,5- tetramethyl- 1,3,2- dioxaborolan-2- yl)nicotinate |

### Intermediate 104 and Intermediate 105

methyl 6′-(3-ethylureido)-4′-(5-(2-methoxyethylcarbamoyl)-4-(trifluoromethyl)thiazol-2-yl)-3,3′-bipyridine-5-carboxylate and 6′-(3-ethylureido)-4′-(5-(2-methoxyethylcarbamoyl)-4-(trifluoromethyl)thiazol-2-yl)-3,3′-bipyridine-5-carboxylic acid

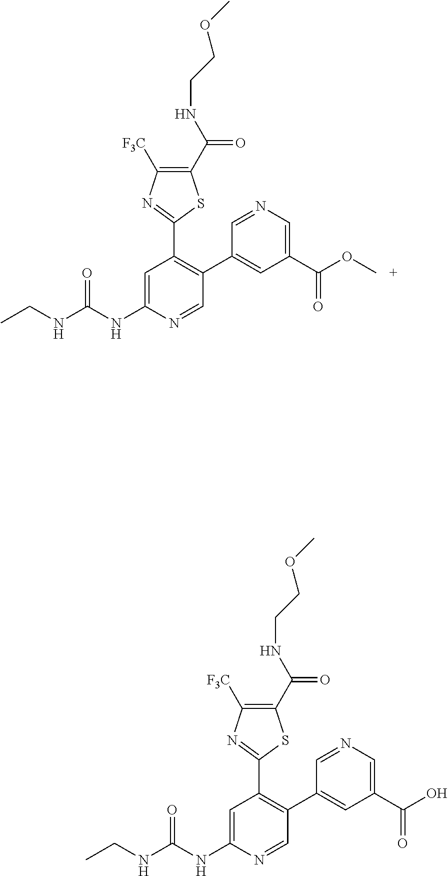

To a 35 mL microwave vial was added sequentially 2-(5-bromo-2-(3-ethylureido)pyridin-4-yl)-N-(2-methoxyethyl)-4-(trifluoromethyl)thiazole-5-carboxamide (Intermediate 83, 0.5 g, 1.0 mmol), methyl 5-(4,4,5,5-tetramethyl-1,3,2-dioxaborolan-2-yl)nicotinate (0.40 g, 1.5 mmol), saturated sodium bicarbonate aqueous solution (3 mL), 1,4-dioxane (10 mL), and dichloro-bis(triphenylphosphino) palladium (II) (70 mg, 0.10 mmol). The mixture was then heated at 110° C. in a microwave for 30 min. Ethyl acetate (40 mL) and water (40 mL) were added. The aqueous layer was then further extracted with ethyl acetate (2×, 50 mL). The combined organics were dried over sodium sulfate, filtered and concentrated. The residue was loaded on 24 g Analogix silica gel column [Heptanes: (9/1) ethyl acetate/methanol] to give ester (Intermediate 105) as off-white powder. The aqueous layer was adjusted to pH ˜4 with dilute HCl and extracted with ethyl acetate/tetrahydrofuran (1/1) (3×, 100 mL). The combined organics were dried over sodium sulfate, filtered, and concentrated to give crude acid Intermediate 106 as yellow solid which was used without further purification.

Intermediate 104: Methyl 6′-(3-ethylureido)-4′-(5-(2-methoxyethylcarbamoyl)-4-(trifluoromethyl)thiazol-2-yl)-3,3′-bipyridine-5-carboxylate

MS (ESP): 553.2 (M+H⁺) for C₂₃H₂₃F₃N₆O₅S

¹H NMR (300 MHz, DMSO-d₆): δ 1.11 (t, 3H), 3.21 (m, 2H), 3.23 (s, 3H), 3.37 (m, 4H), 3.89 (s, 3H), 7.49 (t, 1H), 8.24 (1H), 8.28 (t, 1H), 8.37 (s, 1H), 8.74 (d, 1H), 9.02 (t, 1H), 9.11 (d, 1H), 9.52 (bs, 1H).

Intermediate 105: 6′-(3-ethylureido)-4′-(5-(2-methoxyethylcarbamoyl)-4-(trifluoromethyl)thiazol-2-yl)-3,3′-bipyridine-5-carboxylic acid

MS (ESP): 539.1 (M+H⁺) for C₂₂H₂₁F₃N₆O₅S

### Intermediate 106 and Intermediate 107

methyl 6′-(3-ethylureido)-4′-(5-(2-morpholinoethylcarbamoyl)-4-(trifluoromethyl)thiazol-2-yl)-3,3′-bipyridine-5-carboxylate and 6′-(3-ethylureido)-4′-(5-(2-morpholinoethylcarbamoyl)-4-(trifluoromethyl)thiazol-2-yl)-3,3′-bipyridine-5-carboxylic acid

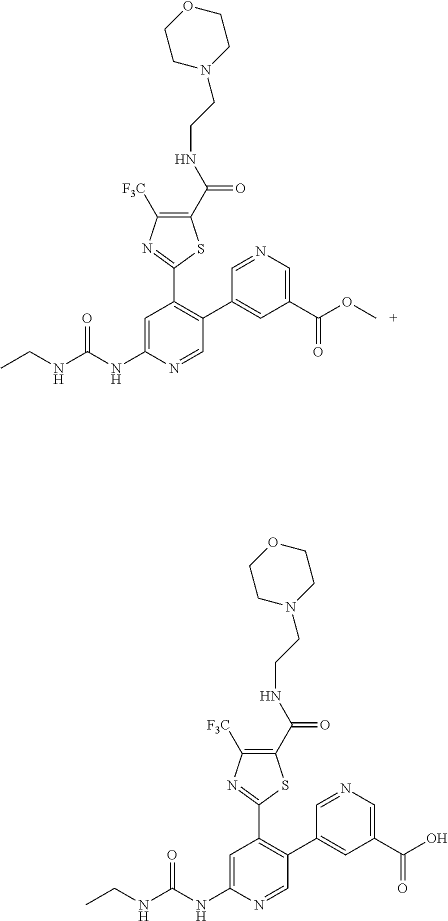

To a 35 mL microwave vial was added sequentially 2-(5-bromo-2-(3-ethylureido)pyridin-4-yl)-N-(2-morpholinoethyl)-4-(trifluoromethyl)thiazole-5-carboxamide (Intermediate 84, 0.5 g, 0.9 mmol), methyl 5-(4,4,5,5-tetramethyl-1,3,2-dioxaborolan-2-yl)nicotinate (0.36 g, 1.4 mmol), saturated sodium bicarbonate aqueous solution (3 mL), 1,4-dioxane (10 mL), and dichloro-bis(triphenylphosphino) palladium (II) (65 mg, 0.09 mmol). The mixture was then heated to 110° C. in a microwave for 30 min. Ethyl acetate (40 mL) and water (40 mL) were added. The aqueous layer was then further extracted with ethyl acetate (2×, 50 mL). The combined organics were dried over sodium sulfate, filtered, and concentrated to give the crude ester (Intermediate 106) which was used without further purification. The aqueous layer was adjusted to pH ˜4 with dilute HCl and extracted with ethyl acetate/tetrahydrofuran (1/1) (3×, 100 mL). The combined organics were dried over sodium sulfate, filtered, and concentrated to give crude acid Intermediate 107 as yellow solid which was also used without further purification.

Intermediate 106: Methyl 6′-(3-ethylureido)-4′-(5-(2-morpholinoethylcarbamoyl)-4-(trifluoromethyl)thiazol-2-yl)-3,3′-bipyridine-5-carboxylate

MS (ESP): 608.1 (M+H⁺) for C₂₆H₂₈F₃N₇O₅S

Intermediate 107: 6′-(3-ethylureido)-4′-(5-(2-morpholinoethylcarbamoyl)-4-(trifluoromethyl)thiazol-2-yl)-3,3′-bipyridine-5-carboxylic acid

MS (ESP): 594.0 (M+H⁺) for C₂₅H₂₆F₃N₇O₅S

### Intermediate 108 and Intermediate 109

methyl 6′-(3-ethylureido)-4′-(5-(2-(4-methylpiperazin-1-yl)ethylcarbamoyl)-4-(trifluoromethyl)thiazol-2-yl)-3,3′-bipyridine-5-carboxylate and 6′-(3-ethylureido)-4′-(5-(2-(4-methylpiperazin-1-yl)ethylcarbamoyl)-4-(trifluoromethyl)thiazol-2-yl)-3,3′-bipyridine-5-carboxylic acid

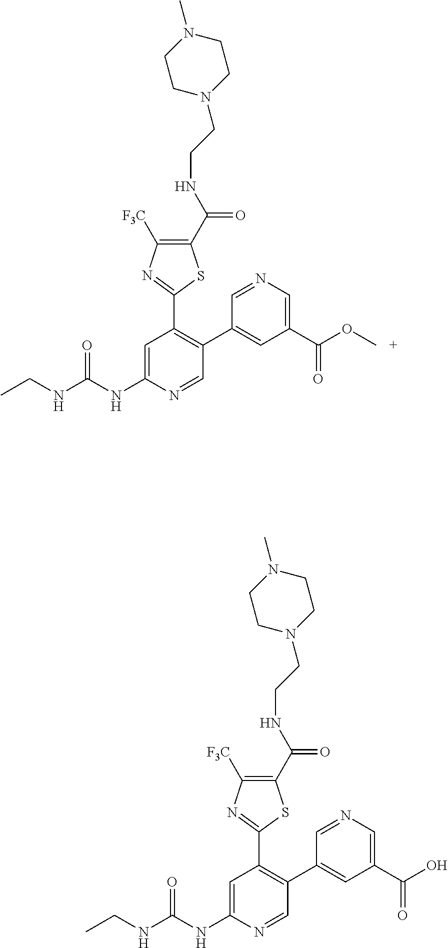

To a 35 mL microwave vial was added sequentially 2-(5-bromo-2-(3-ethylureido)pyridin-4-yl)-N-(2-(4-methylpiperazin-1-yl)ethyl)-4-(trifluoromethyl)thiazole-5-carboxamide (Intermediate 85, 0.5 g, 0.9 mmol), methyl 5-(4,4,5,5-tetramethyl-1,3,2-dioxaborolan-2-yl)nicotinate (0.36 g, 1.4 mmol), saturated sodium bicarbonate aqueous solution (3 mL), 1,4-dioxane (10 mL), and dichloro-bis(triphenylphosphino) palladium (II) (65 mg, 0.09 mmol). The mixture was then heated to 110° C. in a microwave for 30 min. Ethyl acetate (40 mL) and water (40 mL) were added. The aqueous layer was then further extracted with ethyl acetate (2×, 50 mL). The combined organics were dried over sodium sulfate, filtered, and concentrated to give the crude ester (Intermediate 108) which was used for the next step without further purification. The aqueous layer was then adjusted to pH ˜6, 4, and 2 with dilute HCl, and extracted with ethyl acetate/tetrahydrofuran (1/1) however the product remained in the aqueous layer. The aqueous layer was then passed through a 30 g Analogix C18 column (acetonitrile/water) to remove most of the salts and give acid Intermediate 109 as yellow solid which was used without further purification.

Intermediate 108: Methyl 6′-(3-ethylureido)-4′-(5-(2-(4-methylpiperazin-1-yl)ethylcarbamoyl)-4-(trifluoromethyl)thiazol-2-yl)-3,3′-bipyridine-5-carboxylate

MS (ESP): 621.3 (M+H⁺) for C₂₇H₃₁F₃N₈O₄S

Intermediate 109: 6′-(3-ethylureido)-4′-(5-(2-(4-methylpiperazin-1-yl)ethylcarbamoyl)-4-(trifluoromethyl)thiazol-2-yl)-3,3′-bipyridine-5-carboxylic acid

MS (ESP): 607.2 (M+H') for C₂₆H₂₉F₃N₈O₄S

### Intermediate 110 and Intermediate 111

methyl 4′-(5-(cyclopropylcarbamoyl)-4-(trifluoromethyl)thiazol-2-yl)-6′-(3-ethylureido)-3,3′-bipyridine-5-carboxylate and 4′-(5-(cyclopropylcarbamoyl)-4-(trifluoromethyl)thiazol-2-yl)-6′-(3-ethylureido)-3,3′-bipyridine-5-carboxylic acid

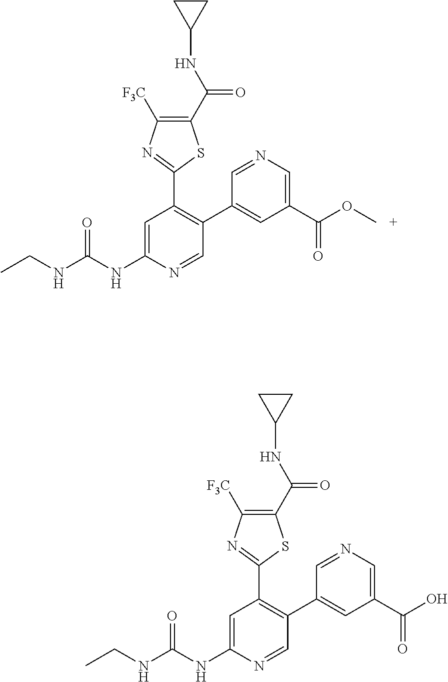

To a 35 mL microwave vial was added sequentially 2-(5-bromo-2-(3-ethylureido)pyridin-4-yl)-N-cyclopropyl-4-(trifluoromethyl)thiazole-5-carboxamide (Intermediate 89, 0.5 g, 1.0 mmol), methyl 5-(4,4,5,5-tetramethyl-1,3,2-dioxaborolan-2-yl)nicotinate (0.4 g, 1.5 mmol), saturated sodium bicarbonate aqueous solution (3 mL), 1,4-dioxane (10 mL), and dichloro-bis(triphenylphosphino) palladium (II) (70 mg, 0.1 mmol). The mixture was then heated to 110° C. in a microwave for 30 min. Ethyl acetate (40 mL) and water (40 mL) were added. The aqueous layer was then further extracted with ethyl acetate (2×, 50 mL). The combined organics were dried over sodium sulfate, filtered, and concentrated to give the crude ester (Intermediate 110) which was used without further purification. The aqueous layer was adjusted to pH ˜4 with dilute HCl and extracted with ethyl acetate/tetrahydrofuran (1/1) (3×, 100 mL). The combined organics were dried over sodium sulfate, filtered, and concentrated to give acid Intermediate 111 as yellow solid which was also used without further purification.

Intermediate 110: Methyl 4′-(5-(cyclopropylcarbamoyl)-4-(trifluoromethyl)thiazol-2-yl)-6′-(3-ethylureido)-3,3′-bipyridine-5-carboxylate

MS (ESP): 535.2 (M+H⁺) for C₂₃H₂₁F₃N₆O₄S

Intermediate 111: 4′-(5-(cyclopropylcarbamoyl)-4-(trifluoromethyl)thiazol-2-yl)-6′-(3-ethylureido)-3,3′-bipyridine-5-carboxylic acid

MS (ESP): 521.1 (M+H⁺) for C₂₂H₁₉F₃N₆O₄S

### Intermediate 112 and Intermediate 113

methyl 4′-(5-(cyclopentylcarbamoyl)-4-trifluomethyl)thiazol-2-yl)-6′-(3-ethylureido)-3,3′-bipyridine-5-carboxylate and 4′-(5-(cyclopentylcarbamoyl)-4-(trifluoromethyl)thiazol-2-yl)-6′-(3-ethylureido)-3,3′-bipyridine-5-carboxylic acid

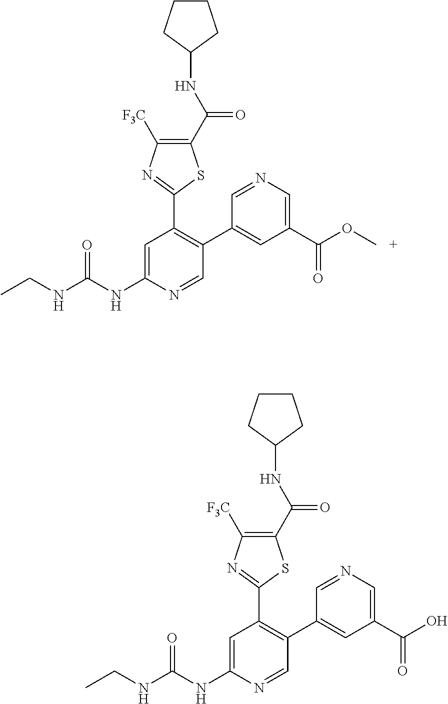

To a 35 mL microwave vial was added sequentially 2-(5-bromo-2-(3-ethylureido)pyridin-4-yl)-N-cyclopentyl-4-(trifluoromethyl)thiazole-5-carboxamide (Intermediate 88, 0.5 g, 1.0 mmol), methyl 5-(4,4,5,5-tetramethyl-1,3,2-dioxaborolan-2-yl)nicotinate (0.4 g, 1.5 mmol), saturated sodium bicarbonate aqueous solution (3 mL), 1,4-dioxane (10 mL), and dichloro-bis(triphenylphosphino) palladium (II) (70 mg, 0.1 mmol). The mixture was then heated to 110° C. in a microwave for 30 min. Ethyl acetate (40 mL) and water (40 mL) were added. The aqueous layer was then further extracted with ethyl acetate (2×, 50 mL). The combined organics were dried over sodium sulfate, filtered, and concentrated to give the crude ester (Intermediate 112) which was used without further purification. The aqueous layer was adjusted to pH ˜4 with dilute HCl and extracted with ethyl acetate/tetrahydrofuran (1/1) (3×, 100 mL). The combined organics were dried over sodium sulfate, filtered, and concentrated to give crude acid Intermediate 113 as yellow solid which was also used without further purification.

Intermediate 112: Methyl 4′-(5-(cyclopentylcarbamoyl)-4-(trifluoromethyl)thiazol-2-yl)-6′-(3-ethylureido)-3,3′-bipyridine-5-carboxylate

MS (ESP): 563.1 (M+H) for C₂₅H₂₅F₃N₆O₄S

Intermediate 113: 4′-(5-(cyclopentylcarbamoyl)-4-(trifluoromethyl)thiazol-2-yl)-6′-(3-ethylureido)-3,3′-bipyridine-5-carboxylic acid

MS (ESP): 549.0 (M+H') for C₂₄H₂₃F₃N₆O₄S

### Intermediate 114 and Intermediate 115

methyl 4′-(5-(cyclohexylcarbamoyl)-4-(trifluoromethyl)thiazol-2-yl)-6′-(3-ethylureido)-3,3′-bipyridine-5-carboxylate and 4′-(5-(cyclohexylcarbamoyl)-4-(trifluoromethyl)thiazol-2-yl)-6′-(3-ethylureido)-3,3′-bipyridine-5-carboxylic acid

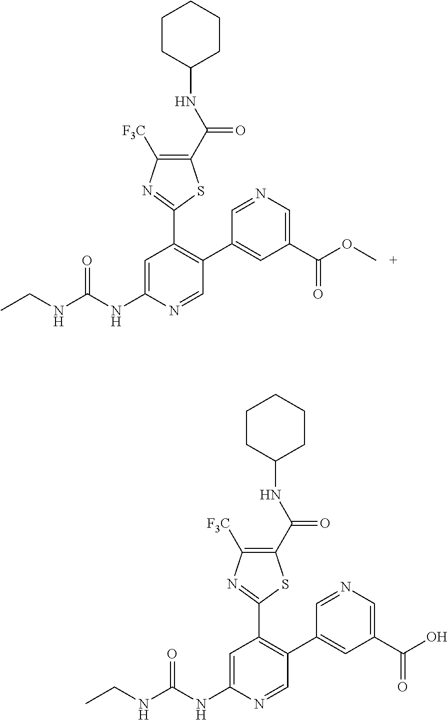

To a 35 mL microwave vial was added sequentially 2-(5-bromo-2-(3-ethylureido)pyridin-4-yl)-N-cyclohexyl-4-(trifluoromethyl)thiazole-5-carboxamide (Intermediate 87, 0.5 g, 1.0 mmol), methyl 5-(4,4,5,5-tetramethyl-1,3,2-dioxaborolan-2-yl)nicotinate (0.4 g, 1.5 mmol), saturated sodium bicarbonate aqueous solution (3 mL) 1,4-dioxane (10 mL), and dichloro-bis(triphenylphosphino) palladium (II) (70 mg, 0.1 mmol). The mixture was then heated to 110° C. in a microwave for 30 min. Ethyl acetate (40 mL) and water (40 mL) were added. The aqueous layer was then further extracted with ethyl acetate (2×, 50 mL). The combined organics were dried over sodium sulfate, filtered, and concentrated to give the crude ester (Intermediate 114) which was used without any further purification. The aqueous layer was adjusted to pH ˜4 with dilute HCl and extracted with ethyl acetate/tetrahydrofuran (1/1) (3×, 100 mL). The combined organics were dried over sodium sulfate, filtered, and concentrated to give crude acid Intermediate 115 as yellow solid which was also used without further purification.

Intermediate 114: Methyl 4′-(5-(cyclohexylcarbamoyl)-4-(trifluoromethyl)thiazol-2-yl)-6′-(3-ethylureido)-3,3′-bipyridine-5-carboxylate

MS (ESP): 577.1 (M+H) for C₂₆H₂₇F₃N₆O₄S

Intermediate 115: 4′-(5-(cyclohexylcarbamoyl)-4-(trifluoromethyl)thiazol-2-yl)-6′-(3-ethylureido)-3,3′-bipyridine-5-carboxylic acid

MS (ESP): 563.1 (M+H⁺) for C₂₅H₂₅F₃N₆O₄S

### Intermediate 116

### Methyl 6-hydroxynicotininate

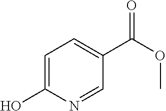

6-Hydroxy-nicotinic acid (100 g, 719 mmol) was suspended in methanol (1 L). 18M Sulfuric acid (50 mL) was added and the reaction was heated at reflux for 16 h. The reaction mixture was then cooled, and sodium bicarbonate powder (45 g) was added slowly to neutralize some of the acid. Most of the methanol was then removed in vacuo. Water (1 L) was added, and the pH adjusted to 7 with the careful addition of bicarbonate solution. The suspension was extracted with dichloromethane (4×, 200 mL), and the organic phases were combined, dried over sodium sulfate, and the solvent was removed in vacuo. The solid was dried in a vacuum oven at 50° C. for 1.5 h to give 83 g (75%) of methyl 6-hydroxynicotinate as a white solid.

MS (ESP): 154.2 (MH⁺) for C₇H₇NO₃

¹H NMR (300 MHz, CDCl₃): 3.88 (s, 3H), 6.59 (dd, 1H), 8.02 (dd, 1H), 8.21 (m, 1H), 13.19 (bs, 1H).

### Intermediate 117

### Methyl 5-bromo-6-hydroxynicotininate

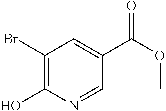

Methyl 6-hydroxynicotininate (Intermediate 116, 50 g, 327 mmol) was suspended in acetic acid (250 mL) and bromine (26.2 mL, 490.5 mmol) was added dropwise to the reaction. The reaction was then heated at 60° C. for 18 h. The reaction mixture was cooled to room temperature, and saturated sodium thiosulfate solution was added to remove remaining bromine. Saturated sodium bicarbonate solution (500 mL) was added slowly, then 1N sodium hydroxide was added carefully until the pH was ˜7. The solid that precipitated were collected by filtration and dried in a vacuum oven at 50° C. for 18 h. This gave 76 g (100%) of methyl 5-bromo-6-hydroxynicotininate as an off white solid.

MS (ESP): 231.9 (MH⁺) for C₇H₆BrNO₃

¹H NMR (300 MHz, DMSO-d₆): 3.80 (s, 3H), 8.12 (s, 1H), 8.19 (s, 1H), 12.77 (bs, 1H).

### Intermediate 118

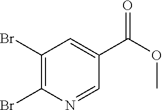

### Methyl 5,6-dibromonicotinate

Methyl 5-bromo-6-hydroxynicotininate (Intermediate 117, 10 g, 43 mmol) was suspended in toluene (100 mL) and phosphorous pentoxide (12 g, 43 mmol) was added. Tetrabutyl ammonium bromide (20 g, 62.1 mmol) was added and the reaction was stirred at reflux for 5 h. The reaction mixture was cooled to ˜50° C. and toluene was decanted from the solution. Toluene (50 mL) was added to the viscous oil and heated to reflux for 30 min. The reaction mixture was cooled to ˜50° C. and toluene was decanted from the solution. This process was repeated twice more, and the toluene extracts were combined. The toluene was washed with saturated bicarbonate (2×, 30 mL), and the solvent was removed in vacuo. The residue was chromatographed on silica gel using 10-50% ethyl acetate in heptane to give 6.3 g (50%) of methyl 5,6-dibromonicotinate as an off white solid.

MS (ESP): 295.8 (MH⁺) for C₇H₅Br₂NO₂

¹H NMR (300 MHz, DMSO-d₆): 3.91 (s, 3H), 8.51 (s, 1H), 8.86 (s, 1H).

### Intermediate 119

### Methyl 5-bromo-6-ethyl nicotinate

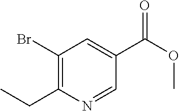

Methyl 5,6-dibromonicotinate (Intermediate 118, 1 g, 3.3 mmol) was dissolved in anhydrous tetrahydrofuran (15 mL) and the reaction was cooled to 0° C. [1,3-Bis(diphenylphosphino)propane]dichloronickel (II) (368 mg, 0.67 mmol) was added, and the solution was stirred for 5 min. Ethyl magnesium bromide (2.0M in tetrahydrofuran, 2.7 mL, 5.4 mmol) was then added dropwise over 30 min keeping the reaction at 0° C. When the addition was complete, the reaction was stirred at 0° C. for 1 h, then water (15 mL) and ethyl acetate (15 mL) were added. The layers were separated, and the aqueous phase was extracted with ethyl acetate (3×, 5 mL). The organic phases were dried over sodium sulfate, filtered and the solvent was removed in vacuo. The residue was chromatographed on a 24 g Analogix column using 0-15% ethyl acetate in heptane. This gave 408 mg (49%) of methyl 5-bromo-6-ethylnicotinate as a white semisolid.

MS (ESP): 243.9 (MH⁺) for C₇H₅Br₂NO₂

¹H NMR (300 MHz, CDCl₃): 1.33 (t, 1H), 3.08 (q, 2H), 3.92 (s, 3H), 8.35 (d, 1H), 9.01 (d, 1H).

### Intermediate 120

### methyl 2-ethyl-6′-(3-ethylureido)-4′-(4-(trifluoromethyl)thiazol-2-yl)-3,3′-bipyridine-5-carboxylate

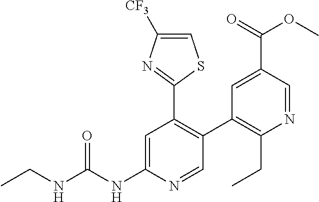

To a slurry of 6-(3-ethylureido)-4-(4-trifluoromethylthiazol-2-yl)pyridin-3-ylboronic acid (Intermediate 12, 410 mg, 1.1 mmol), methyl 5-bromo-6-ethylnicotinate (Intermediate 119, 140 mg, 0.57 mmol) and trans dichlorobis(triphenylphosphine)palladium (II) (40 mg, 0.06 mmol) in 1,2-dimethoxyethane (12 mL) was added a solution of sodium bicarbonate (143 mg, 1.7 mmol) in water (3 mL). The reaction was stirred for 45 min at 125° C. in the microwave. The reaction mixture was cooled to room temperature, and ethyl acetate (20 mL) and water (10 mL) were added to help separate the layers. The water was removed, and the organic phase was washed with water (3 mL). The reaction was then concentrated and subjected to silica gel chromatography on a 12 g Analogix column using 0-100% ethyl acetate in heptane. This gave 60 mg (21%) of methyl 2-ethyl-6′-(3-ethylureido)-4′-(4-(trifluoromethyl)thiazol-2-yl)-3,3′-bipyridine-5-carboxylate as an of white solid.

MS (ESP): 480.0 (MH⁺) for C₂₁H₂₀F₃N₅O₃S

¹H NMR (300 MHz, DMSO-d₆): 0.98 (t, 3H), 1.11 (t, 3H), 2.39-2.51 (m, 2H), 3.18-3.28 (m, 2H), 3.92 (s, 3H), 7.61 (bt, 1H), 8.07 (d, 1H), 8.24 (s, 1H), 8.37 (s, 1H), 8.45 (s, 1H), 9.09 (d, 1H), 9.42 (bs, 1H).

### Intermediate 121

The following compound was prepared according to the procedure for Intermediate 120 from the starting materials indicated in the Table.

| Int | Compound | Data | SM |
|---|---|---|---|
| 121 | methyl 2-ethyl-6′-(3-ethylureido)-4′-(4- phenylthiazol-2-yl)-3,3′-bipyridine-5- carboxylate 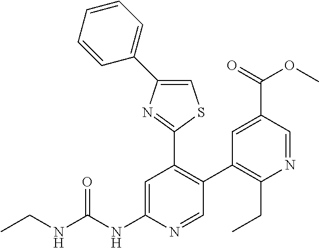 | MS (ESP): 488.1 (MH⁺) for C₂₆H₂₅N₅O₃S | Intermediate 161 and Intermediate 119 |

### Intermediates 122-123

The following Intermediates were prepared by the general procedure as described below from the starting materials indicated in the Table.

### General Procedure

An ethyl ester (0.1 mmol) was suspended in 1:1 methanol:tetrahydrofuran (6 mL) and 1N sodium hydroxide (3 mL) was added. The reaction was stirred at room temperature for 16 h then concentrated under reduced pressure to remove the organic solvents to get a thin slurry. This slurry was acidified to pH ˜3 with 1N hydrochloric acid. This suspension was filtered and washed with water (3 mL) and dichloromethane (3 mL). The solid (or paste) was dried in a vacuum oven to give the product acid.

| Int | Compound | Data | SM |
|---|---|---|---|
| 122 | 2-ethyl-6′-(3-ethylureido)-4′-(4- (trifluoromethyl)thiazol-2-yl)-3,3′- bipyridine-5-carboxylic acid 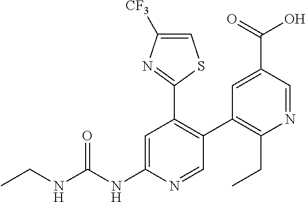 | MS (ESP): 465.9 (MH⁺) for C₂₀H₁₈F₃N₅O₃S ¹H NMR (300 MHz, DMSO-d₆): 0.97 (t, 3H), 1.13 (t, 3H), 2.39-2.54 (m, 2H), 3.16-3.27 (m, 2H), 7.43 (bt, 1H), 8.06 (d, 1H), 8.29 (s, 1H), 8.36 (s, 1H), 8.49 (s, 1H), 9.11 (d, 1H), 9.44 (bs, 1H) | Intermediate 120 |
| 123 | 2-ethyl-6′-(3-ethylureido)-4′-(4- phenylthiazol-2-yl)-3,3′-bipyridine-5- carboxylic acid 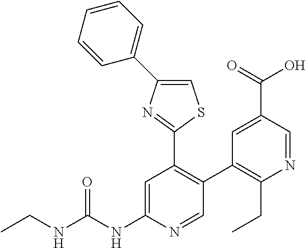 | MS (ESP): 474.0 (MH⁺) for C₂₅H₂₃N₅O₃S | Intermediate 121 |

### Intermediate 124

### 2-chloro-6-ethoxypyridin-4-amine

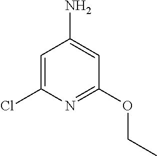

To a sealed tube was charge 2,6-dichloro-4-aminopyridine (5 g, 30.7 mmol), and sodium ethoxide (21 wt %, 9.92 g) with anhydrous ethanol (3 mL). The mixture was heated at 145° C. for 2 h in a microwave. Water was added, the crude product was extracted with ethyl acetate (3×), and the combined organic layers were dried over sodium sulfate, filtered and concentrated. During concentration, a crystalline solid precipitated from the crude to give clean product (2.4 g, 45.4%). The filtrate was purified (chromatography heptane/ethyl acetate) and more product (1.5 g, 25.6%) was obtained.

MS (ESP): 173.1 (MH⁺) for C₇H₉ClN₂O

¹H NMR (300 MHz, CD₃OD): δ 1.3 (t, 3H), 4.6 (q, 2H), 5.8 (d, 1H), 6.2 (d, 1H).

### Intermediate 125

### methyl 4-amino-6-ethoxypicolinate

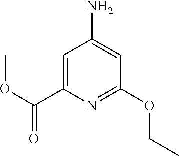

To a 2 L Parr Bomb was charged 2-chloro-6-ethoxypyridin-4-amine (Intermediate 124, 3.7 g, 21.4 mmol) and methanol (300 mL). [1,1′-bis(diphenylphosphino)ferrocene]dichloropalladium (II) complex with dichloromethane (870 mg, 5 mol %) was added followed by triethylamine (6 mL) and the resulting mixture was heated at 100° C. under 100 psi CO atmosphere for 2 d. The reaction mixture was cooled to room temperature and the mixture was concentrated to dryness and directly purified by Analogix in hexane/ethyl acetate to give a light brown solid (3.7 g, 88.7%).

MS (ESP): 197.1 (MH⁺) for C₉H₁₂N₂O₃

¹H NMR (300 MHz, CD₃OD): δ 1.36 (t, 3H), 3.85 (s, 3H), 4.22 (q, 2H), 6.05 (d, 1H), 7.0 (d, 1H).

### Intermediate 126

### methyl 4-chloro-6-ethoxypicolinate

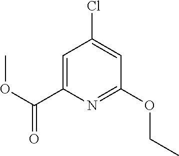

To a 1 L round bottom flask was charged t-butyl nitrite (1.55 mL, 11.48 mmol) with acetonitrile (200 mL), then copper (II) chloride (640 mg, 4.58 mmol) was added, and the mixture was allowed to heat at 70° C. to give a dark green solution. Methyl 4-amino-6-ethoxypicolinate (Intermediate 125, 1.51 g, 7.64 mmol) was added and gas evolution was observed. The mixture was heated at 70° C. for 1 h. After cooling to room temperature, water was added and the mixture was extracted with ethyl acetate (3×). The combined organic layers were washed with brine, ammonium chloride solution, and dried over sodium sulfate.

After concentration, the crude product was purified by Analogix (heptane/ethyl acetate 0-30%) to give a white solid (1.45 g, 88.4%).

MS (ESP): 216.0 (MH⁺) for C₉H₁₀ClNO₃

¹H NMR (300 MHz, CD₃OD): δ 1.38 (t, 3H), 4.0 (s, 3H), 4.42 (q, 2H), 7.05 (d, 1H), 7.65 (d, 1H).

### Intermediate 127

### 2-chloro-6-isopropoxypyridin-4-amine

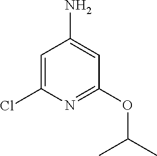

To a 500 mL sealed tube was charge 2,6-dichloro-4-aminopyridine (10.9 g, 66.9 mmol) with isopropanol (300 mL) and sodium hydride (95%, 9 g, 335 mmol). The mixture was heated at 150° C. for 2 d. Water was added, the crude product was extracted with ethyl acetate (3×), and the combined organic layers were dried over sodium sulfate. After concentration under reduced pressure, the crude mixture was directly used for the carbonylation without further purification.

MS (ESP): 186.9 (MH⁺) for C₈H₁₁ClN₂O.

### Intermediate 128

### methyl 4-amino-6-isopropoxypicolinate

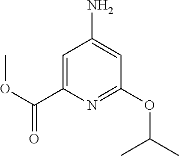

To a 2 L Parr Bomb was charged 2-chloro-6-isopropoxypyridin-4-amine (Intermediate 127, 12.5 g, 66.9 mmol) with methanol (300 mL). [1,1′-Bis(diphenylphosphino)ferrocene]dichloropalladium (II) complex with dichloromethane (2.80 g, 5 mol %) was added followed by triethylamine (18.8 mL). The resulting mixture was heated at 100° C. under 100 psi CO atmosphere for overnight. The mixture was concentrated to dryness and directly purified by Analogix in hexane/ethyl acetate to give a light yellow solid (10.3 g, 74%).

MS (ESP): 211.2 (MH⁺) for C₁₀H₁₄N₂O₃

¹H NMR (300 MHz, CD₃OD): δ 1.27 (d, 6H), 3.94 (s, 3H), 5.12-5.20 (m, 1H), 6.03 (d, 1H), 7.00 (d, 1H).

### Intermediate 129

### methyl 4-chloro-6-isopropoxypicolinate

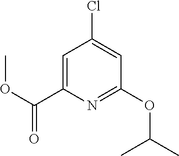

To a 1 L round bottom flask was charged t-butyl nitrite (6 mL, 44 mmol) with acetonitrile (200 mL), then copper (II) chloride (2.44 g, 17.2 mmol) was added, and the mixture was allowed to heat at 70° C. for 30 min to give a dark green solution. Methyl 4-amino-6-isopropoxypicolinate (Intermediate 128, 6 g, 28.6 mmol) was added and gas evolution was observed. The mixture was heated at 70° C. for 1 h. After cooling to room temperature, water was added and the mixture extracted with ethyl acetate (3×). The combined organic layers were washed with brine, ammonium chloride solution, and dried over sodium sulfate. After concentration, the crude product was purified by Analogix (heptane/ethyl acetate 0-50%) to give a light yellow liquid (4.33 g, 66%).

MS (ESP): 230.1 (MH⁺) for C₁₀H₁₂ClNO₃

¹H NMR (300 MHz, CD₃OD): δ 1.25 (d, 6H), 3.9 (s, 3H), 5.4 (heptat, 1H), 7.00 (d, 1H), 7.60 (d, 1H).

### Intermediate 130

### 2-chloro-6-(cyclopropylmethoxy)pyridin-4-amine

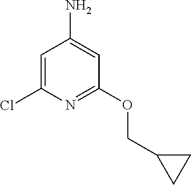

To a 500 mL sealed tube was charge 2,6-dichloro-4-aminopyridine (10 g, 61.3 mmol) with cyclopropylmethanol (200 mL) and sodium hydride (95%, 3.2 g, 122.9 mmol). The reaction mixture was heated at 150° C. for overnight. After cooling to room temperature, water was added, the crude product was extracted with ethyl acetate (3×), and the combined org. layers were dried over sodium sulfate. After concentration, the crude mixture was purified by Analogix (heptane/ethyl acetate 0-40%) to give a white solid (12 g, 98.4%).

MS (ESP): 199.2 (MH⁺) for C₉H₁₁ClN₂O

¹H NMR (300 MHz, CD₃OD): δ ppm 0.30-0.34 (m, 2H), 0.54-0.60 (m, 2H), 1.16-1.25 (m, 1H), 3.94 (d, 2H), 5.83 (d, 1H), 6.22 (d, 1H).

### Intermediate 131

### methyl 4-amino-6-(cyclopropylmethoxy)picolinate

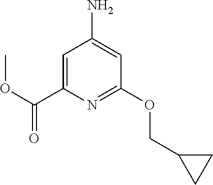

To a 2 L Parr Bomb was charged 2-chloro-6-(cyclopropylmethoxy)pyridin-4-amine (Intermediate 130, 9 g, 45.3 mmol.) with methanol (300 mL). [1,1′-Bis(diphenylphosphino)ferrocene]dichloropalladium (II) complex with dichloromethane (1.5 g, 6 mol %) was added followed by triethylamine (13 mL). The resulting mixture was heated at 100° C. under 100 psi CO atmosphere for 2 d. The mixture was concentrated to dryness and directly purified by Analogix in hexane/ethyl acetate system to give a light yellow solid (8.7 g, 61.4%).

MS (ESP): 223.2 (MH⁺) for C₁₁H₁₄N₂O₃

¹H NMR (300 MHz, CD₃OD): δ 0.30-0.33 (m, 2H), 0.54-0.57 (m, 2H), 1.21-1.25 (m, 1H), 3.87 (s, 3H), 4.03 (d, 2H), 4.88 (s, 2H), 6.06 (d, 2H), 7.02 (d, 2H).

### Intermediate 132

### methyl 4-chloro-6-(cyclopropylmethoxy)picolinate

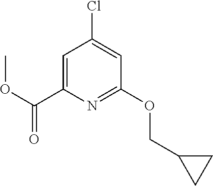

To a 1 L round bottom flask wash charged t-butyl nitrite (5.5 mL, 40.5 mmol) with acetonitrile (200 mL), then copper (II) chloride (2.26 g, 16.2 mmol) was added. The mixture was allowed to heat at 70° C. for 30 min to give a dark greenish solution. Methyl 4-amino-6-(cyclopropylmethoxy)picolinate (Intermediate 131, 6 g, 27 mmol) was added and gas evolution was observed. The mixture was heated at 70° C. for 1.5 h. After cooling to room temperature, water was added and the mixture was extracted with ethyl acetate (3×). The combined organic layers were washed with brine, ammonium chloride solution, and dried over sodium sulfate. After concentration, the crude product was purified by Analogix (heptane/ethyl acetate 0-30%) to give a light yellow liquid (4.46 g, 68.5%).

MS (ESP): 242.1 (MH⁺) for C₁₁H₁₂ClNO₃

¹H NMR (300 MHz, CD₃OD): δ 0.34-0.39 (m, 2H), 0.54-0.62 (m, 2H), 1.22-1.33 (m, 1H), 4.21 (d, 2H), 7.04 (d, 1H), 7.65 (d, 1H).

### Intermediate 133

### 2-chloro-6-morpholinopyridin-4-amine

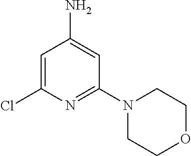

To a 500 mL sealed tube was charge 2,6-dichloro-4-aminopyridine (10 g, 61.3 mmol) with morpholine (11 mL) and 1,4-dioxane (50 mL). After heating at 150° C. for overnight the reaction was incomplete. More morpholine (11 mL) was added and the reaction again heated at 150° C. for overnight. After cooling to room temperature, water was added and the crude product was extracted with ethyl acetate (3×). The combined organic layers were dried over sodium sulfate. After concentration, the crude mixture was purified by Analogix (heptane/ethyl acetate 0-40%) to give a white solid (11.5 g, 87.8%).

MS (ESP): 214.2 (MH⁺) for C₉H₁₂ClN₃O

¹H NMR (300 MHz, CD₃OD): δ 3.30 (t, 4H), 3.68 (t, 4H), 5.8 (d, 1H), 6.0 (d, 1H).

### Intermediate 134

### methyl 4-amino-6-morpholinopicolinate

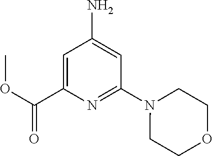

To a 2 L Parr Bomb was charged 2-chloro-6-morpholinopyridin-4-amine (Intermediate 133, 11 g, 51.4 mmol.) with methanol (300 mL). [1,1′-Bis(diphenylphosphino)ferrocene]dichloropalladium (II) complex with dichloromethane (2.11 g, 5 mol %) was added followed by triethylamine (15 mL). The resulting mixture was heated at 100° C. under 100 psi CO atmosphere for 2 d. The mixture was filtered through a Celite pad and the filtrate was washed with water and extracted with ethyl acetate (3×). The combined organic layers were dried over sodium sulfate. After concentration, the crude mixture was purified by Analogix (tetrahydrofuran/ethyl acetate 0-40%) to give an off-white solid (8.4 g, 68.7%).

MS (ESP): 238.2 (MH⁺) for C₁₁H₁₅N₃O₃

¹H NMR (300 MHz, CD₃OD): δ 3.42 (t, 4H), 3.74 (t, 4H), 3.86 (s, 3H), 6.09 (d, 1H), 6.84 (d, 1H).

### Intermediate 135

### methyl 4-chloro-6-morpholinopicolinate

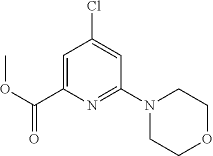

To a 1 L round bottom flask wash charged t-butyl nitrite (3.6 mL, 27 mmol) with acetonitrile (200 mL), then copper (II) chloride (1.4 g, 10.08 mmol) was added, and the reaction mixture was allowed to heat at 70° C. for 30 min to give a dark greenish solution. Methyl 4-amino-6-morpholinopicolinate (Intermediate 134, 4 g, 16.8 mmol) was added and gas evolution was observed. The mixture was heated at 70° C. for 0.5 h. After cooling to room temperature, water was added and the mixture was extracted with ethyl acetate (3×). The combined organic layers were washed with brine, ammonium chloride solution, and dried over sodium sulfate. After concentration, the crude product was purified by Analogix (heptane/ethyl acetate 0-30%) to give a yellow liquid (2.6 g, 60.2%).

MS (ESP): 257.1 (MH⁺) for C₁₁H₁₃ClN₂O₃

¹H NMR (300 MHz, CD₃OD): δ 3.6 (t, 4H), 3.8 (t, 4H), 3.9 (s, 3H), 7.05 (d, 1H), 7.4 (d, 1H).

### Intermediate 136

### 2-chloro-6-(4-methylpiperazin-1-yl)pyridin-4-amine

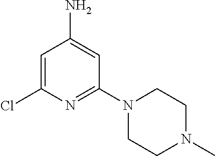

To a 500 mL sealed tube was charge 2,6-dichloro-4-aminopyridine (10 g, 61.3 mmol) with 1-methylpiperazine (8.4 mL) and 1,4-dioxane (50 mL). After heating at 170° C. for overnight the reaction was incomplete. More 1-methylpiperazine (12.6 mL) was added and the reaction heated at 170° C. for 2 d. After cooling to room temperature, water was added and the crude product was extracted with ethyl acetate (3×). Most of the bis-(1-methylpiperazine) by-product stayed in the aqueous layer. The combined organic layers were dried over sodium sulfate and after concentration the crude was used for the carbonylation.

MS (ESP): 227.1 (MH⁺) for C₁₀H₁₅ClN.

### Intermediate 137

### methyl 4-amino-6-(4-methylpiperazin-1-yl)picolinate

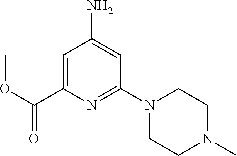

To a 2 L Parr Bomb was charged 2-chloro-6-(4-methylpiperazin-1-yl)pyridin-4-amine (Intermediate 136, 61.3 mmol) with methanol (300 mL). [1,1′-Bis(diphenylphosphino)ferrocene]dichloropalladium (II) complex with dichloromethane (2.5 g, 5 mol %) was added followed by triethylamine (17 mL). The mixture was heated at 100° C. under 100 psi CO atmosphere overnight. The reaction mixture was triturated by dichloromethane to give a product as grey solid at 90% purity (6.5 g, 42.5% in two steps).

MS (ESP): 251.1 (MH⁺) for C₁₂H₁₈N₄O₂

¹H NMR (300 MHz, CD₃OD): δ 2.9 (s, 3H), 3.3 (t, 4H), 3.9 (s, 3H), 4.9 (t, 4H), 6.2 (d, 1H), 6.9 (d, 1H).

### Intermediate 138

### methyl 4-chloro-6-(4-methylpiperazin-1-yl)picolinate

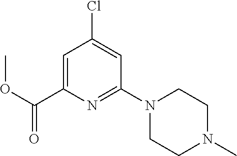

To a 1 L round bottom flask was charged t-butyl nitrite (2 mL, 15.3 mmol) with acetonitrile (200 mL), then copper (II) chloride (850 mg, 6.12 mmol) was added, and the mixture was allowed to heat at 70° C. for 30 min to give a dark greenish solution. Methyl 4-amino-6-(4-methylpiperazin-1-yl)picolinate (Intermediate 137, 2.55 g, 10.2 mmol) was added and gas evolution was observed. The mixture was heated at 70° C. for a further 2 h. After cooling to room temperature, the mixture was filtered through a Celite pad and the filtrate was concentrated. The crude product was purified by Analogix (heptane/ethyl acetate 0-30%) to give a white solid.

MS (ESP): 270.0 (MH⁺) for C₁₂H₁₆ClN₃O₂

¹H NMR (300 MHz, CD₃OD): δ 2.95 (s, 3H), 3.55-3.65 (m, 4H), 3.90 (s, 3H), 4.6-4.7 (m, 4H), 7.22 (d, 1H), 7.45 (d, 1H).

### Intermediate 139

### 2-chloro-6-(1-methylpiperidin-4-yloxy)pyridin-4-amine

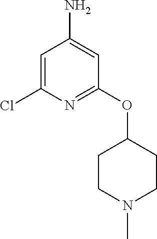

To a 500 mL sealed tube was charge 2,6-dichloro-4-aminopyridine (10 g, 61.3 mmol), sodium hydride (60% in mineral oil, 6.1 g, 153.37 mmol) with 1-methyl-4-hydroxypiperidine (25 g, 217 mmol). Toluene (50 mL, anhydrous) was added to aid transferring 1-methyl-4-hydroxypiperidine. Bubbling in the reaction mixture was observed on addition of the piperidine. When the gas evolution ceased, the reaction mixture was heated at 120° C. for 2 h. More sodium hydride (1.4 g, 35 mmol) was added and heating continued at 120° C. for another 1.5 h. After cooling to room temperature, water was added and the crude product was extracted with dichloromethane/isopropanol (2:1) three times. The combined organic layers were dried over sodium sulfate. After concentration, the crude was used for the carbonylation.

MS (ESP): 227.1 (MH⁺) for C₁₁H₁₆ClN₃O.

### Intermediate 140

### methyl 4-amino-6-(1-methylpiperidin-4-yloxy)picolinate

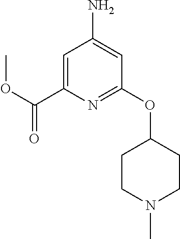

To a 2 L Parr Bomb was charged 2-chloro-6-(4-methylpiperazin-1-yl)pyridin-4-amine (Intermediate 139, 61.3 mmol) with methanol (300 mL). [1,1′-Bis(diphenylphosphino)ferrocene]dichloropalladium (II) complex with dichloromethane (2.5 g, 5 mol %) was added followed by triethylamine (17 mL). The mixture was heated at 100° C. under 100 psi CO atmosphere overnight. The crude reaction was filtered through Celite and the filtrate was concentrated to dryness. The crude product was directly purified by chromatography (˜2% (2M ammonia in methanol) in dichloromethane) to give a brown solid (4.75 g, 29.2%)

MS (ESP): 266.1 (MH⁺) for C₁₃H₁₉N₃O₃

¹H NMR (300 MHz, CD₃OD): δ 1.7-1.9 (m, 2H), 2.35 (s, 3H), 2.7-2.8 (m, 2H), 3.95 (s, 3H), 5.0-5.1 (m, 1H), 6.1 (s, 1H), 7.0 (s, 1H).

### Intermediate 141

### methyl 4-bromo-6-(1-methylpiperidin-4-yloxy)picolinate

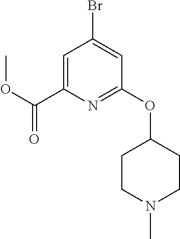

To a 1 L round bottom flask was charged methyl 4-amino-6-(1-methylpiperidin-4-yloxy)picolinate (Intermediate 140, 2.75 g, 10.4 mmol) with acetonitrile (200 mL). Then t-butyl nitrite (2.1 mL, 15.6 mmol) was added at 45° C. followed by copper (II) bromide (1.16 g, 5.19 mmol), and the dark greenish mixture heated at 45° C. for 2 h. After cooling to room temperature, the mixture was filtered through a Celite pad and the filtrate concentrated under reduced pressure. The crude product was directly purified by Analogix (dichloromethane/methanol) to give a light yellow solid (1 g, 29.4%).

MS (ESP): 329.1 (MH⁺) for C₁₃H₁₇BrN₂O₃

¹H NMR (300 MHz, CD₃OD): δ 2.05-2.15 (br, 4H), 2.9 (s, 3H), 3.25-3.45 (br, 4H), 3.96 (s, 3H), 5.4-5.5 (m, 1H), 7.3 (s, 1H), 7.9 (s, 1H).

### Intermediate 142

### 2-chloro-6-(2-(dimethylamino)ethoxy)pyridin-4-amine

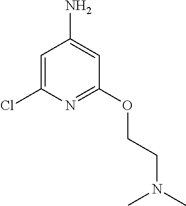

To a 500 mL sealed tube was charge 2,6-dichloro-4-aminopyridine (10.1 g, 62 mmol), sodium hydride (95%, 3.2 g, 126.8 mmol) and N,N-dimethylethanolamine (50 mL). After heating at 170° C. for overnight the reaction was incomplete. More sodium hydride (0.5 g, 19.8 mmol) was added and heating continued at 170° C. for another 1.5 h. After cooling to room temperature, water was added and the crude product was extracted with dichloromethane/isopropanol (2:1) three times. The combined organic layers were dried over sodium sulfate, filtered, concentrated under reduced pressure and the crude directly used for carbonylation.

MS (ESP): 216.0 (MH⁺) for C₉H₁₄ClN₃O.

### Intermediate 143

### methyl 4-amino-6-(2-(dimethylamino)ethoxy)picolinate

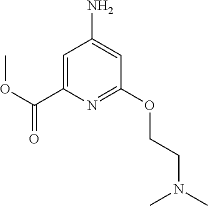

To a 2 L Parr Bomb was charged 2-chloro-6-(2-(dimethylamino)ethoxy)pyridin-4-amine (Intermediate 142, 62 mmol) with methanol (300 mL). [1,1′-Bis(diphenylphosphino)ferrocene]dichloropalladium (II) complex with dichloromethane (2.53 g, 5 mol %) was added followed by triethylamine (17.3 mL). The resulting mixture was heated at 100° C. under 100 psi CO atmosphere overnight. The mixture was concentrated to dryness and washed with water and brine, the mixture was extracted with dichloromethane/isopropanol (2:1) and ethanol/tetrahydrorfuran (1:1). The organic layers were combined and dried over sodium sulfate. After concentration, the crude product was purified by chromatography (˜2% (2M ammonia in methanol) in dichloromethane) to give a brown sticky solid. (8.5 g, 57%)

MS (ESP): 240.3 (MH⁺) for C₁₁H₁₇N₃O₃

¹H NMR (300 MHz, CD₃OD): δ 2.4 (s, 6H), 2.8 (t, 2H), 3.9 (s, 3H), 4.4 (t, 2H), 6.1 (s, 1H), 7.1 (s, 1H).

### Intermediate 144

### methyl 4-bromo-6-(2-(dimethylamino)ethoxy)picolinate

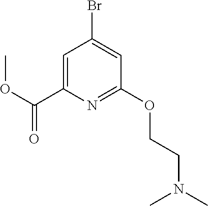

To a 1 L round bottom flask was charged methyl 4-amino-6-(2-(dimethylamino)ethoxy)picolinate (Intermediate 143, 1.35 g, 5.6 mmol) with acetonitrile (100 mL), then t-butyl nitrite (1.2 mL, 8.5 mmol) was added. The mixture was heated at 50° C. for ˜10 min, then copper (II) bromide (1.16 g, 5.19 mmol) was added and the mixture heated at 50° C. for a further 2 h. After cooling to room temperature, the mixture was filtered through a Celite pad and the filtrate was concentrated. The crude product was purified by Analogix (dichloromethane/methanol) to give a light yellow solid (350 mg, 20.6%).

MS (ESP): 305.1 (MH⁺) for C₁₁H₁₅BrN₂O₃

### Intermediate 145

### methyl 6′-ethoxy-6-(3-ethylureido)-4-(4-(trifluoromethyl)thiazol-2-yl)-3,4′-bipyridine-2′-carboxylate

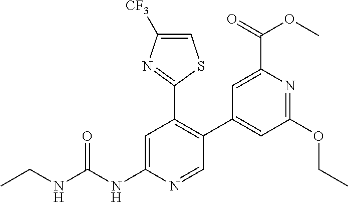

To a microwave sealed tube was charged methyl 4-chloro-6-ethoxypicolinate (Intermediate 126, 500 mg, 2.33 mmol) and [1,1′-bis(diphenylphosphino)ferrocene]dichloropalladium (II) complex with dichloromethane (96 mg, 0.116 mmol) with dioxane (10 mL). Sodium bicarbonate (390 mg, 4.65 mmol), water (2 mL) were added, then 6-(3-ethylureido)-4-(4-(trifluoromethyl)thiazol-2-yl)pyridin-3-ylboronic acid (Intermediate 12, 920 mg, 5.12 mmol) was added, and the mixture was purged with N₂ for ˜5 min. The resulting mixture was heated to 80° C. for 0.5 h. The mixture was cooled to room temperature, diluted with water, extracted with ethyl acetate (3×) and the combined organic layers were dried over sodium sulfate. After concentration, the crude mixture was purified by Analogix (heptane/ethyl acetate 0-50%) to give a white solid (300 mg, 26%).

MS (ESP): 496.2 (MH⁺) for C₂₁H₂₀H₂₀F₃N₅O₄S. Intermediate 146

### methyl 6′-ethoxy-6-(3-ethylureido)-4-(4-phenylthiazol-2-yl)-3,4′-bipyridine-2′-carboxylate

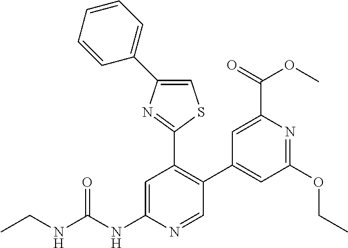

To a microwave sealed tube was charged methyl 4-chloro-6-ethoxypicolinate (Intermediate 126, 470 mg, 2.19 mmol), [1,1′-bis(diphenylphosphino)ferrocene]dichloropalladium (II) complex with dichloromethane (53 mg, 0.0649 mmol) and 1,4-dioxane (8 mL). Then sodium bicarbonate (374 mg, 4.45 mmol), water (2 mL) and 6-(3-ethylureido)-4-(4-phenylthiazol-2-yl)pyridin-3-ylboronic acid (Intermediate 161, 820 g, 2.23 mmol) were added and the reaction mixture purged with N₂ for 10 min. The resulting mixture was heated to 80° C. for 0.5 h. The reaction mixture was cooled to room temperature, diluted with water, extracted with ethyl acetate (3×) and the combined organic layers dried over sodium sulfate. After concentration under reduced pressure, the crude mixture was purified by Analogix (heptane/ethyl acetate 0-60%) to give an off-white solid (650 mg).

MS (ESP): 504.0 (MH⁺) for C₂₆H₂₅N₅O₄S

### Intermediate 147

### methyl 6-(3-ethylureido)-6′-isopropoxy-4-(4-(trifluoromethyl)thiazol-2-yl)-3,4′-bipyridine-2′-carboxylate

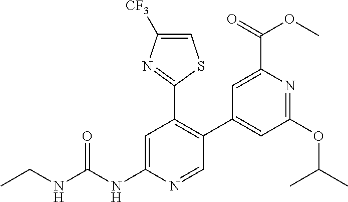

To a microwave sealed tube was charged methyl 4-chloro-6-isopropoxypicolinate (Intermediate 129, 510 mg, 2.23 mmol) and tetrakis(triphenylphosphine)palladium (0) (129 mg, 0.111 mmol) with 1,4-dioxane (12 mL). Then sodium bicarbonate (390 mg, 4.65 mmol), water (3 mL) and 6-(3-ethylureido)-4-(4-(trifluoromethyl)thiazol-2-yl)pyridin-3-ylboronic acid (Intermediate 12, 821 mg, 2.28 mmol) was added and the mixture purged with N₂ for 5 min. The resulting mixture was heated to 80° C. for 0.5 h. The reaction mixture was cooled to room temperature, diluted with water, and extracted with ethyl acetate (3×). The combined organic layers were dried over sodium sulfate then purified by Analogix (heptane/ethyl acetate 0-100%) to give a light brown solid (900 mg).

MS (ESP): 510.0 (MH⁺) for C₂₂H₂₂F₃N₅O₄S

### Intermediate 148

### methyl 6-(3-ethylureido)-6′-isopropoxy-4-(4-phenylthiazol-2-yl)-3,4′-bipyridine-2′-carboxylate

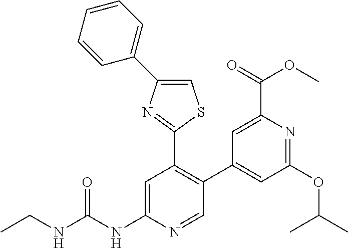

To a microwave sealed tube was charged methyl 4-chloro-6-isopropoxypicolinate (Intermediate 129, 1 g, 4.36 mmol) and tetrakis(triphenylphosphine)palladium (0) (252 mg, 0.21 mmol) with 1,4-dioxane (24 mL). Sodium bicarbonate (540 mg, 8.8 mmol), water (6 mL), and 6-(3-ethylureido)-4-(4-phenylthiazol-2-yl)pyridin-3-ylboronic acid (Intermediate 161, 1.62 g, 4.38 mmol) were added. The mixture was purged with N₂ for ˜5 min then heated to 80° C. for 0.5 h. The reaction mixture was cooled to room temperature, diluted with water, and extracted with ethyl acetate (3×). The combined organic layers were dried over sodium sulfate. After concentration, the crude mixture was triturated with ethanol to give a bright yellow solid which was a mixture of the desired methyl ester and de-boronated compound (1.1 g).

MS (ESP): 518.1 (MH⁺) for C₂₇H₂₇N₅O₄S

### Intermediate 149

### methyl 6′-(cyclopropylmethoxy)-6-(3-ethylureido)-4-(4-(trifluoromethyl)thiazol-2-yl)-3,4′-bipyridine-2′-carboxylate

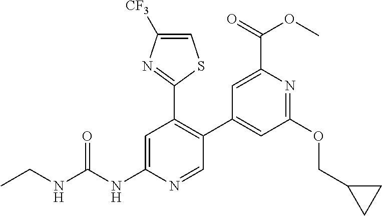

To a microwave sealed tube was charged methyl 4-chloro-6-(cyclopropylmethoxy)picolinate (Intermediate 132, 435 mg, 1.81 mmol), 6-(3-ethylureido)-4-(4-(trifluoromethyl)thiazol-2-yl)pyridin-3-ylboronic acid (Intermediate 12, 650 mg, 1.81 mmol) and 1,4-dioxane (12 mL). Then sodium bicarbonate (305 mg, 2.64 mmol) and water (3 mL) were added and the mixture was purged by N₂ for 5 min. Tetrakis(triphenylphosphine)palladium (0) (104 mg, 0.095 mmol) was added, and the resulting mixture was heated to 80° C. for 1 h. The reaction mixture was cooled to room temperature, diluted with water, and extracted with ethyl acetate (3×). The combined organic layers were dried over sodium sulfate. After concentration under reduced pressure, the crude mixture was triturated by ethanol to give a light brown solid (100 mg).

MS (ESP): 522.1 (MH⁺) for C₂₃H₂₂F₃N₅O₄S

### Intermediate 150

### methyl 6′-(cyclopropylmethoxy)-6-(3-ethylureido)-4-(4-phenylthiazol-2-yl)-3,4′-bipyridine-2′-carboxylate

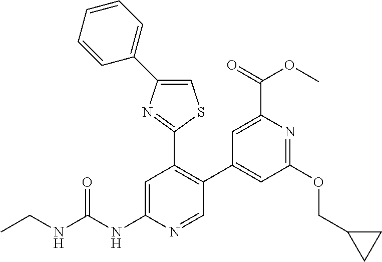

To a microwave sealed tube was charged methyl 4-chloro-6-(cyclopropylmethoxy)picolinate (Intermediate 132, 328 mg, 1.36 mmol), 6-(3-ethylureido)-4-(4-phenylthiazol-2-yl)pyridin-3-ylboronic acid (Intermediate 161, 510 mg, 1.38 mmol) and 1,4-dioxane (12 mL). Sodium bicarbonate (305 mg, 2.64 mmol) was added with water (3 mL) and the mixture was purged by N₂ for ˜5 min. Tetrakis(triphenylphosphine)palladium (0) (82 mg, 0.071 mmol) was added and the resulting mixture was heated to 80° C. for 1 h. The mixture was cooled to room temperature, diluted with water and extracted with ethyl acetate (3×). The combined organic layers were dried over sodium sulfate. After concentration under reduced pressure, the crude mixture was used without purification.

MS (ESP): 530.1 (MH⁺) for C₂₈H₂₇N₅O₄S

### Intermediate 151

### methyl 6-(3-ethylureido)-6′-morpholino-4-(4-(trifluoromethyl)thiazol-2-yl)-3,4′-bipyridine-2′-carboxylate

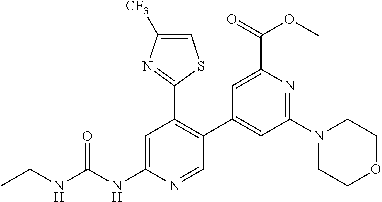

To a microwave sealed tube was charged methyl 4-chloro-6-morpholinopicolinate (Intermediate 135, 357 mg, 1.39 mmol), 6-(3-ethylureido)-4-(4-(trifluoromethyl)thiazol-2-yl)pyridin-3-ylboronic acid (Intermediate 12, 500 mg, 1.39 mmol) and 1,4-dioxane (15 mL). Sodium bicarbonate (240 mg, 2.86 mmol) was added with water (3 mL), the mixture was purged by N₂ for 10 min, then tetrakis(triphenylphosphine)palladium (0) (90 mg, 0.078 mmol) was added. The resulting mixture was heated to 80° C. for 1 h. The reaction mixture was cooled to room temperature, diluted with water, and extracted with ethyl acetate (3×). The combined organic layers were dried over sodium sulfate. After concentration under reduced pressure, a brown solid was obtained and triturated with ethanol (cold) to give an off-white solid (350 mg, 54%).

MS (ESP): 537.0 (MH⁺) for C₂₃H₂₃F₃N₆O₄S

### Intermediate 152

### 6-(3-ethylureido)-6′-morpholino-4-(4-phenylthiazol-2-yl)-3,4′-bipyridine-2′-carboxylate

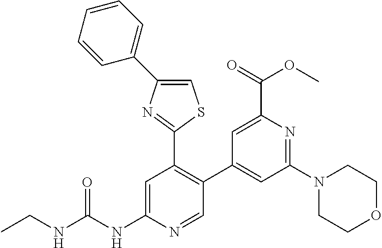

To a microwave sealed tube was charged methyl 4-chloro-6-morpholinopicolinate (Intermediate 135, 492 g, 1.91 mmol), 6-(3-ethylureido)-4-(4-phenylthiazol-2-yl)pyridin-3-ylboronic acid (Intermediate 161, 700 mg, 1.91 mmol) with 1,4-dioxane (12 mL). Sodium bicarbonate (320 mg, 3.81 mmol) was added with water (3 mL), the mixture was purged by N₂ for 10 min, then tetrakis(triphenylphosphine)palladium (0) (80 mg, 0.069 mmol) was added. The resulting mixture was heated to 85° C. for 1 h. The reaction was incomplete so more 6-(3-ethylureido)-4-(4-phenylthiazol-2-yl)pyridin-3-ylboronic acid (230 mg) was added with more tetrakis(triphenylphosphine)palladium (0) (84 mg, 0.073 mmol) and the resulting mixture was heated at 85° C. for two more hours. The mixture was cooled to room temperature, diluted with water, and extracted with ethyl acetate (3×). The combined organic layers were dried over sodium sulfate. After concentration, a yellow solid (700 mg) was obtained which is the mixture of the desired methyl ester, de-boronated compound and homocoupling product

MS (ESP): 545.1 (MH⁺) for C₂₈H₂₈N₆O₄S

### Intermediate 153

### methyl 6-(3-ethylureido)-6′-(1-methylpiperidin-4-yloxy)-4-(4-(trifluoromethyl)thiazol-2-yl)-3,4′-bipyridine-2′-carboxylate

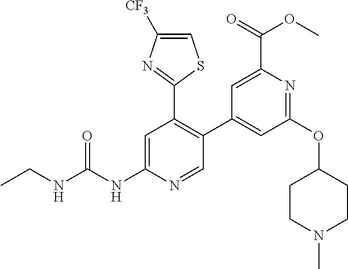

To a microwave sealed tube was charged methyl 4-bromo-6-(1-methylpiperidin-4-yloxy)picolinate (Intermediate 141, 400 mg, 1.22 mmol), 6-(3-ethylureido)-4-(4-(trifluoromethyl)thiazol-2-yl)pyridin-3-ylboronic acid (Intermediate 12, 800 mg, 2.22 mmol) and 1,4-dioxane (12 mL). Then K₃PO₄ solution (2 N in water, 1.4 mL), and tetrakis(triphenylphosphine)palladium (0) (140 mg, 0.121 mmol) were added and the mixture was purged by N₂ for ˜10 min. The resulting mixture was heated to 90° C. for 1 h. LC showed starting bromide remained so more 6-(3-ethylureido)-4-(4-(trifluoromethyl)thiazol-2-yl)pyridin-3-ylboronic acid (200 mg) was added and the mixture was heated at 90° C. for a further 1 h. The mixture was concentrated to dryness and triturated with methyl tert-butyl ether. The filtrate was concentrated and triturated by methyl tert-butyl ether again. The second filtrate was concentrated and purified by Analogix (dichloromethane/methanol) to give a light yellow solid (250 mg, 36.3%)

MS (ESP): 565.2 (MH⁺) for C₂₅H₂₇F₃N₆O₄S

### Intermediate 154

### methyl 6′-(2-(dimethylamino)ethoxy)-6-(3-ethylureido)-4-(4-(trifluoromethyl)thiazol-2-yl)-3,4′-bipyridine-2′-carboxylate

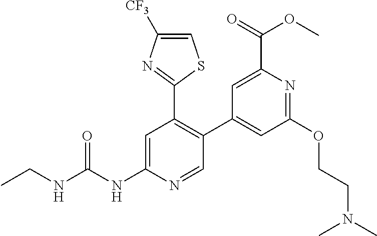

To a microwave sealed tube was charged methyl 4-bromo-6-(2-(dimethylamino)ethoxy)picolinate (Intermediate 144, 300 mg, 0.987 mmol), 6-(3-ethylureido)-4-(4-(trifluoromethyl)thiazol-2-yl)pyridin-3-ylboronic acid (Intermediate 12, 426 mg, 1.18 mmol) with 1,4-dioxane (12 mL). Then K₃PO₄ solution (2 N in water, 1.2 mL) was added with tetrakis(triphenylphosphine)palladium (0) (115 mg, 0.099 mmol), and the mixture purged by N₂ for 10 min. The resulting mixture was heated to 90° C. for 1 h. The reaction mixture was cooled to room temperature and some solid precipitated and was filtered to give de-boronated by-product. The filtrate was diluted with water and extracted with ethyl acetate (3×). The organic layers were combined and dried over sodium sulfate, after concentration, the crude product was triturated by dichloromethane twice to remove most of the less soluble de-boronated product. The resulting filtrate was concentrated and purified by Analogix (dichloromethane/methanol) to give a light yellow sticky solid (120 mg).

MS (ESP): 539.1 (MH⁺) for C₂₃H₂₅F₃N₆O₄S

### Intermediate 155

### 1-ethyl-3-(2′-(hydrazinecarbonyl)-4-(4-phenylthiazol-2-yl)-3,4′-bipyridin-6-yl)urea

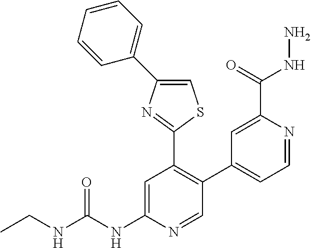

Intermediate 155 was synthesized according to the procedure described for Intermediate 22 from Intermediate 158 and hydrazine.

LC/MS (ES⁺)[(M+H)⁺]: 460 for C₂₃H₂₁N₇O₂S.

¹H NMR (300 MHz, d₆-DMSO): 1.12 (t, 3H), 3.22 (m, 2H), 4.58 (s, 2H), 7.34-7.43 (m, 3H), 7.54 (d, 1H), 7.74 (d, 3H), 7.91 (s, 1H), 8.2 (d, 2H), 8.3 (s, 1H), 8.6 (d, 1H), 9.5 (s, 1H), 10.0 (s, 1H).

### Intermediate 156

### 1-ethyl-3-(5-(5-(hydrazinecarbonyl)-4-(1-methyl-1H-1,2,4-triazol-5-yl)thiazol-2-yl)-4-(4-phenylthiazol-2-yl)pyridin-2-yl)urea

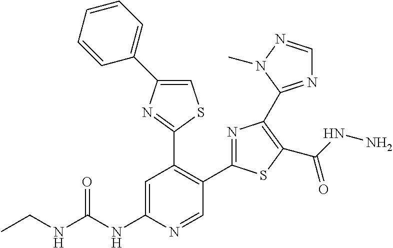

Intermediate 156 was synthesized according to the procedure described for Intermediate 22 from Intermediate 159 and hydrazine.

LC/MS (ES⁺)[(M+H)^{+]}: 547 for C₂₄H₂₂N₁₀O₂S₂.

¹H NMR (300 MHz, d₆-DMSO): 1.11 (t, 3H), 3.21 (m, 2H), 3.93 (s, 3H), 4.75 (d, 2H), 7.37 (m, 3H), 7.56 (m, 1H), 7.83 (d, 2H), 8.11 (s, 1H), 8.24 (s, 1H), 8.36 (s, 1H), 8.79 (s, 1H), 9.67 (s, 1H), 11.84 (s, 1H).

### Intermediate 157

### 1-ethyl-3-(5-(5-(hydrazinecarbonyl)-4-(pyrimidin-2-yl)thiazol-2-yl)-4-(4-phenylthiazol-2-yl)pyridin-2-yl)urea

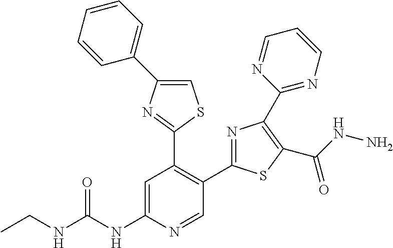

Intermediate 157 was synthesized according to the procedure described for Intermediate 22 from Intermediate 160 and hydrazine.

LC/MS (ES⁺)[(M+H)^{+]}: 544 for C₂₅H₂₁N₉O₂S₂.

### Intermediate 158-160

The following intermediates were prepared in accordance to the procedure described for Intermediate 20 using the starting materials indicated in the table.

| Int | Compound | Structure | Data | SM |
|---|---|---|---|---|
| 158 | methyl 6-(3- ethylureido)- 4-(4- phenylthiazol- 2-yl)-3,4′- bipyridine-2′- carboxylate | 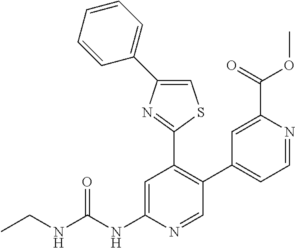 | LC/MS (ES⁺)[(M+H)⁺]: 460 for C₂₄H₂₁N₅O₃S. ¹H NMR (300 MHz, DMSO-d6): 1.11(t, 3H), 3.21 (q, 2H), 3.84 (s, 3H), 7.35 (m, 3H), 7.63 (m, 4H), 8.01 (s, 1H), 8.20 (d, 2H), 8.30 (s, 1H), 8.7 (d, 1H), 9.5 (s, 1H) | Intermediate 161 and methyl 4- bromopicolinate |
| 159 | methyl 2-(6- (3- ethylureido)- 4-(4- phenylthiazol- 2-yl)pyridin- 3-yl)-4-(1- methyl-1H- 1,2,4-triazol- 5-yl)thiazole- 5-carboxylate | 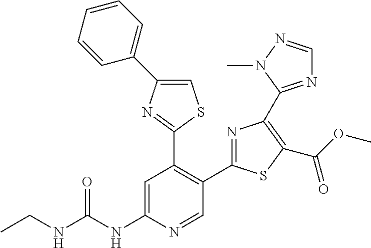 | LC/MS (ES⁺)[(M+H)⁺]: 547 for C₂₅H₂₂N₈O₃S₂. ¹H NMR (300 MHz, CDCl3): 1.11 (t, 3H), 3.21 (q, 2H), 3.6 (s, 3H), 3.75 (s, 3H), 7.4 (m, 5H), 7.84 (d, 2H), 8.05 (s, 1H), 8.17 (s, 1H), 8.38 (s, 1H), 8.76 (s, 1H), 9.70 (s, 1H) | Intermediate 161 and Intermediate 44 |
| 160 | ethyl 2-(6-(3- ethylureido)- 4-(4- phenylthiazol- 2-yl)pyridin- 3-yl)-4- (pyrimidin-2- yl)thiazole-5- carboxylate | 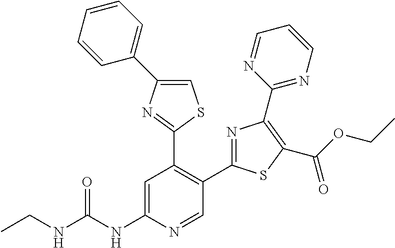 | LC/MS (ES⁺)[(M + H)⁺]: 558 for C₂₇H₂₃N₇O₃S₂. | Intermediate 161 and Intermediate 43 |

### Intermediate 161

### 6-(3-ethylureido)-4-(4-phenylthiazol-2-yl)pyridin-3-ylboronic acid

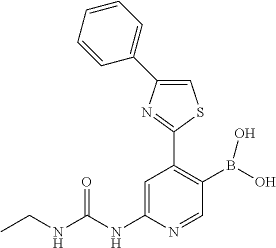

A solution of 1-(5-bromo-4-(4-phenylthiazol-2-yl)pyridin-2-yl)-3-ethylurea (2.97 g, 7.36 mmol, Intermediate 16) in THF (25 mL) was cooled to −78° C. Isopropylmagnesium chloride, 2.0M in THF (8.84 mL, 17.67 mmol) was added slowly and the reaction was slowly warmed to −15° C. before being cooled back down to −78° C. N-Butyllithium, 2.5M in hexanes (14.73 mL, 36.82 mmol) was then added and the reaction was stirred at −78° C. for 1 h. Trimethyl borate (8.21 mL, 73.64 mmol) was added all at once and an exotherm was observed. Following the exotherm, the reaction mixture was allowed to warm to room temperature and stir for 3 h. The reaction mixture was then cooled to 0° C. and 20 mL of water was added slowly followed by 10 mL of 6N HCl. The reaction mixture was allowed to warm to room temperature and stir for 30 min. The reaction mixture was concentrated under reduced pressure to remove THF. The aqueous portion was diluted with 1N NaOH and diethylether. The aqueous layer was acidified with HCl and the resulting precipitate was the title compound.

MS (ESP): 369 (M+H⁺) for C₁₇H₁₇BN₄O₃S.

¹H NMR (DMSO-d₆): δ 1.1 (t, 3H), 3.2 (q, 2H), 7.4-7.5 (m, 3H), 7.8 (s, 1H), 7.9 (s, 1H), 8.1 (d, 2H), 9.3 (s, 1H).

### Intermediate 162

### Methyl 6′-(3-ethylureido)-2-fluoro-4′-(4-(trifluoromethyl)thiazol-2-yl)-3,3′-bipyridine-5-carboxylate

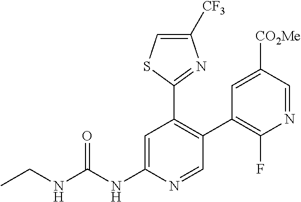

The 6-(3-ethylureido)-4-(4-(trifluoromethyl)thiazol-2-yl)pyridin-3-ylboronic acid (Intermediate 12, 1.20 g, 3.33 mmol), methyl 5-bromo-6-fluoronicotinate (WO200224681, 0.819 g, 3.50 mmol), tris(dibenzylideneacetone)dipalladium(0) (0.305 g, 0.33 mmol), and 2-dicyclohexylphosphino-2′,4′,6′-triisopropylbiphenyl (0.477 g, 1.00 mmol) were combined and then degassed and purged with N₂ twice. A solution of sodium carbonate (0.353 g, 3.33 mmol) in water (4.5 mL) was added, followed by the addition of acetonitrile (18 mL). The flask was degassed and purged with N₂ again. The mixture was heated at 80° C. for 1.5 h and then stirred at RT overnight. The mixture was conc in vacuo, diluted with EtOAc and water and filtered through a fitted funnel fitted with filter paper. The layers of the filtrate were separated. The organic layer was washed three times with sat NH₄Cl, once with brine, dried over Na₂SO₄, and conc in vacuo. Purification via silica gel chromatography (50% acetone/hexanes; then 5-10% MeOH/CH₂Cl₂) gave 0.351 g (22%) of the title compound.

LC/MS (ES⁺)(M+H)⁺: 470 for C₁₉H₁₅F₄N₅O₃S

¹H NMR (DMSO-d₆): δ 9.56 (s, 1H); 8.82 (m, 1H); 8.61 (s, 1H); 8.49 (m, 1H); 8.40 (s, 1H); 8.25 (s, 1H); 7.50 (m, 1H); 3.90 (s, 3H); 3.21 (m, 2H); 1.11 (t, 3H).

### Intermediate 163 and Intermediate 164

### Methyl 6′-(3-ethylureido)-2-(2-(4-methylpiperazin-1-yl)ethoxy)-4′-(4 (trifluoromethyl)thiazol-2-yl)-3,3′-bipyridine-5-carboxylate and 2-(4-Methylpiperazin-1-yl)ethyl 6′-(3-ethylureido)-2-carboxylate

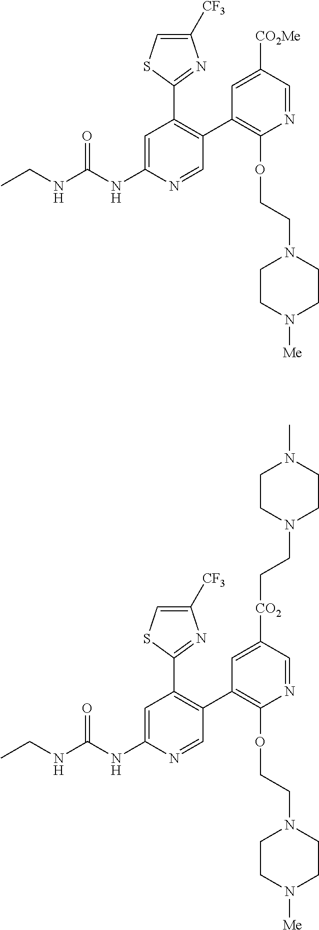

The 2-(4-methylpiperazin-1-yl)ethanol (0.154 g, 1.07 mmol) in THF (0.5 mL) was cooled to 0° C. A 1.0 M solution of lithium bis(trimethylsilyl)amide in THF (1.066 mL, 1.07 mmol) was added dropwise. The mixture was stirred at 0° C. for 10 min and then stirred at RT for 15 min. This mixture was then added dropwise to a 0° C. solution of methyl 6′-(3-ethylureido)-2-fluoro-4′-(4-(trifluoromethyl)thiazol-2-yl)-3,3′-bipyridine-5-carboxylate (Intermediate 162, 0.115 g, 0.24 mmol) in THF (1 mL). Additional THF (0.5 mL) was added. The resultant mixture was stirred at 0° C. for 10 min and then stirred at RT for 30 min. The mixture was cooled to 0° C., quenched with satd NH₄Cl and conc in vacuo. The residue was diluted with EtOAc and water and the layers were separated. The organic layer was washed with satd NH₄Cl, water, brine, dried over Na₂SO₄ and conc in vacuo. LC/MS indicated a mixture of methyl 6′-(3-ethylureido)-2-(2-(4-methylpiperazin-1-yl)ethoxy)-4′-(4-(trifluoromethyl)thiazol-2-yl)-3,3′-bipyridine-5-carboxylate (Intermediate 163) and 2-(4-methylpiperazin-1-yl)ethyl 6′-(3-ethylureido)-2-(2-(4-methylpiperazin-1-yl)ethoxy)-4′-(4-(trifluoromethyl)thiazol-2-yl)-3,3′-bipyridine-5-carboxylate (Intermediate 164) which was used without further purification.

Intermediate 163: LC/MS (ES⁺)[(M+H)⁺]: 594 for C₂₆H₃₀F₃N₇O₄S

Intermediate 164: LC/MS (ES⁺)[(M+H)⁺]: 706 for C₃₂H₄₂F₃N₉O₄S

### Intermediate 165

### 1-Ethyl-3-(5′-(hydrazinecarbonyl)-2′-(2-(4-methylpiperazin-1-yl)ethoxy)-4-(4-(trifluoromethyl)thiazol-2-yl)-3,3′-bipyridin-6-yl)urea

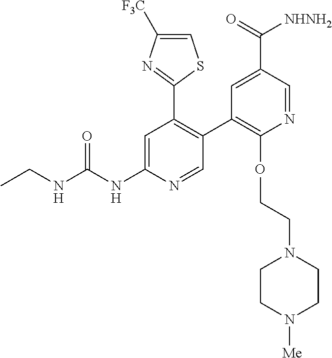

Hydrazine hydrate (0.117 mL, 2.40 mmol) was added to 142 mg of a mixture of methyl 6′-(3-ethylureido)-2-(2-(4-methylpiperazin-1-yl)ethoxy)-4′-(4-(trifluoromethyl)thiazol-2-yl)-3,3′-bipyridine-5-carboxylate (Intermediate 163) and 2-(4-methylpiperazin-1-yl)ethyl 6′-(3-ethylureido)-2-(2-(4-methylpiperazin-1-yl)ethoxy)-4′-(4-(trifluoromethyl)thiazol-2-yl)-3,3′-bipyridine-5-carboxylate (Intermediate 164). The reaction mixture was heated at 82° C. overnight. After conc in vacuo, the residue was diluted with EtOAc and water and the layers were separated. The organic layer was washed three times with satd NH₄Cl, once with brine, dried over Na₂SO₄ and conc in vacuo to give the title compound which was used without further purification.

LC/MS (ES⁺)[(M+H)⁺]: 594 for C₂₅H₃₀F₃N₉O₃S.

### Intermediate 166 and Intermediate 167

Methyl 2-(2-(dimethylamino)ethoxy)-6′-(3-ethylureido)-4′-(4-(trifluoromethyl)thiazol-2-yl)-3,3′-bipyridine-5-carboxylate and 2-(Dimethylamino)ethyl 2-(2-(dimethylamino)ethoxy)-6′-(3-ethylureido)-4′-(4-(trifluoromethyl)thiazol-2-yl)-3,3′-bipyridine-5-carboxylate

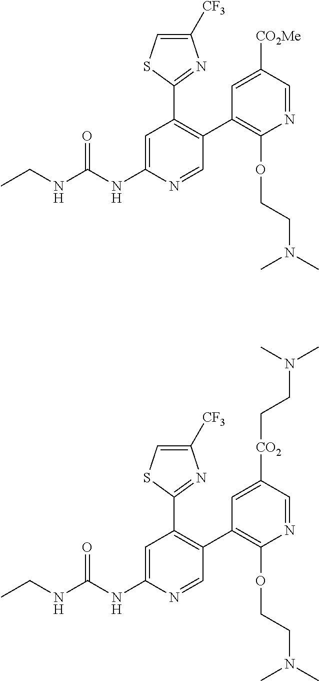

Following the procedure for Intermediates 163 and Intermediate 164, 2-(dimethylamino)ethanol (0.1 mL, 1.04 mmol) and methyl 6′-(3-ethylureido)-2-fluoro-4′-(4-(trifluoromethyl)thiazol-2-yl)-3,3′-bipyridine-5-carboxylate (Intermediate 162, 0.112 g, 0.24 mmol) were reacted to give a mixture of methyl 2-(2-(dimethylamino)ethoxy)-6′-(3-ethylureido)-4′-(4-(trifluoromethyl)thiazol-2-yl)-3,3′-bipyridine-5-carboxylate (Intermediate 166) and 2-(dimethylamino)ethyl 2-(2-(dimethylamino)ethoxy)-6′-(3-ethylureido)-4′-(4-(trifluoromethyl)thiazol-2-yl)-3,3′-bipyridine-5-carboxylate (Intermediate 167) which was used without further purification.

Intermediate 166: LC/MS (ES⁺)[(M+H)⁺]: 539 for C₂₃H₂₅F₃N₆O₄S

Intermediate 167: LC/MS (ES⁺)[(M+H)⁺]: 596 for C₂₆H₃₂F₃N₇O₄S

### Intermediate 168

### 1-(2′-(2-(Dimethylamino)ethoxy)-5′-(hydrazinecarbonyl)-4-(4-(trifluoromethyl)thiazol-2-yl)-3,3′-bipyridin-6-yl)-3-ethylurea

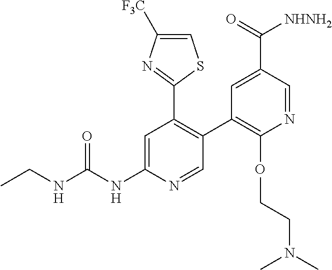

Following the procedure for Intermediate 165, 0.129 g of the mixture of methyl 2-(2-(dimethylamino)ethoxy)-6′-(3-ethylureido)-4′-(4-(trifluoromethyl)thiazol-2-yl)-3,3′-bipyridine-5-carboxylate (Intermediate 166) and 2-(dimethylamino)ethyl 2-(2-(dimethylamino)ethoxy)-6′-(3-ethylureido)-4′-(4-(trifluoromethyl)thiazol-2-yl)-3,3′-bipyridine-5-carboxylate (Intermediate 167) was reacted to give the title compound which was used without further purification.

LC/MS (ES⁺)[(M+H)⁺]: 539 for C₂₂H₂₅F₃N₈O₃S.

### Intermediate 169

### Methyl 6′-(3-ethylureido)-2-methoxy-4′-(4-(trifluoromethyl)thiazol-2-yl)-3,3′-bipyridine-5-carboxylate

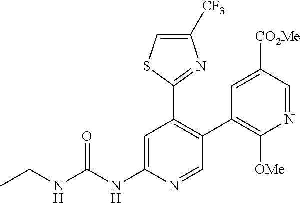

The methyl 6′-(3-ethylureido)-2-fluoro-4′-(4-(trifluoromethyl)thiazol-2-yl)-3,3′-bipyridine-5-carboxylate (Intermediate 162, 0.121 g, 0.26 mmol) was suspended in THF (4 mL) and cooled to 0° C. A 0.5 M solution of sodium methoxide in MeOH (2.243 mL, 1.12 mmol) was added dropwise. The mixture was stirred at 0° C. for 20 min and then warmed to RT. After quenching with satd NH₄Cl, the mixture was conc in vacuo. The residue was diluted with EtOAc and water and the layers were separated. The organic layer was washed with water, brine, dried over Na₂SO₄ and conc in vacuo to give the title compound which was used without further purification.

LC/MS (ES⁺)[(M+H)⁺]: 482 for C₂₀H₁₈F₃N₅O₄S.

### Intermediate 170

### 1-Ethyl-3-(5′-(hydrazinecarbonyl)-2′-methoxy-4-(4-(trifluoromethyl)thiazol-2-yl)-3,3′-bipyridin-6-yl)urea

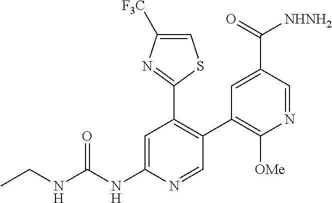

Following the procedure for Intermediate 165, methyl 6′-(3-ethylureido)-2-methoxy-4′-(4-(trifluoromethyl)thiazol-2-yl)-3,3′-bipyridine-5-carboxylate (Intermediate 169, 0.197 g, 0.41 mmol) was reacted to give the title compound which was used without further purification.

LC/MS (ES⁺)[(M+H)⁺]: 482 for C₁₉H₁₈F₃N₇O₃S.

### Intermediate 171 and Intermediate 172

Methyl 6′-(3-ethylureido)-2-(2-morpholinoethoxy)-4′-(4-(trifluoromethyl)thiazol-2-yl)-3,3′-bipyridine-5-carboxylate and 2-Morpholinoethyl 6′-(3-ethylureido)-2-(2-morpholinoethoxy)-4′-(4-(trifluoromethyl)thiazol-2-yl)-3,3′-bipyridine-5-carboxylate

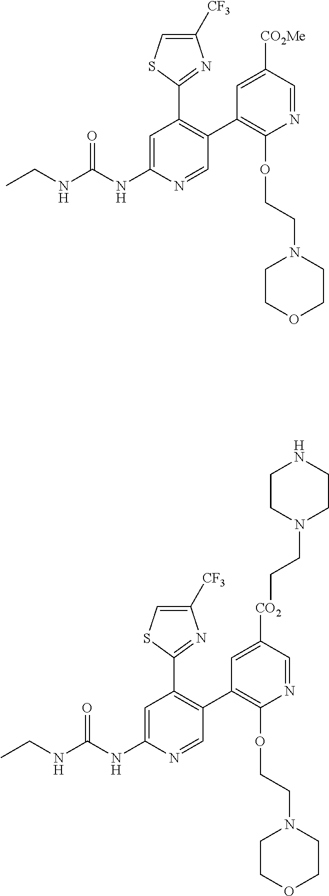

Following the procedure for Intermediates 163 and Intermediate 164, 2-morpholinoethanol (0.08 mL, 0.66 mmol) and methyl 6′-(3-ethylureido)-2-fluoro-4′-(4-(trifluoromethyl)thiazol-2-yl)-3,3′-bipyridine-5-carboxylate (Intermediate 162, 0.071 g, 0.15 mmol) were reacted to give a mixture of methyl 6′-(3-ethylureido)-2-(2-morpholinoethoxy)-4′-(4-(trifluoromethyl)thiazol-2-yl)-3,3′-bipyridine-5-carboxylate (Intermediate 171) and 2-morpholinoethyl 6′-(3-ethylureido)-2-(2-morpholinoethoxy)-4′-(4-(trifluoromethyl)thiazol-2-yl)-3,3′-bipyridine-5-carboxylate (Intermediate 172) which was used without further purification.

Intermediate 171: LC/MS (ES⁺)[(M+H)⁺]: 581 for C₂₅H₂₇F₃N₆O₅S

Intermediate 172: LC/MS (ES⁺)[(M+H)⁺]: 680 for C₃₀H₃₆F₃N₇O₆S

### Intermediate 173

### 1-Ethyl-3-(5′-(hydrazinecarbonyl)-2′-(2-morpholinoethoxy)-4-(4-trifluoromethyl)thiazol-2-yl)-3,3′-bipyridin-6-yl)urea

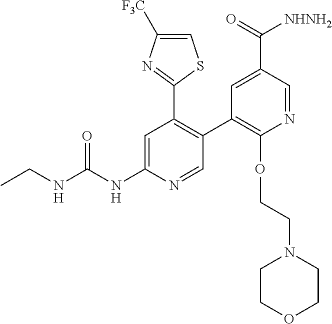

Following the procedure for Intermediate 165, 0.087 g of the mixture of methyl 6′-(3-ethylureido)-2-(2-morpholinoethoxy)-4′-(4-(trifluoromethyl)thiazol-2-yl)-3,3′-bipyridine-5-carboxylate (Intermediate 171) and 2-morpholinoethyl 6′-(3-ethylureido)-2-(2-morpholinoethoxy)-4′-(4-(trifluoromethyl)thiazol-2-yl)-3,3′-bipyridine-5-carboxylate (Intermediate 172) was reacted to give the title compound which was used without further purification.

LC/MS (ES⁺)[(M+H⁺]: 581 for C₂₄H₂₇F₃N₈O₄S.

### Intermediate 174

### 6-(3-Ethylureido)-4-(4-(pyridin-2-yl)thiazol-2-yl)pyridin-3-ylboronic acid

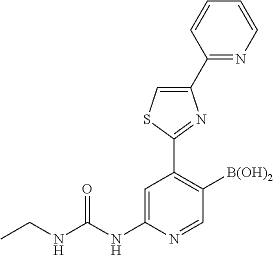

The N-[5-bromo-4-(4-(pyridin-2-yl)thiazol-2-yl)pyridin-2-yl]-N′-ethylurea (Intermediate 15, 0.965 g, 2.39 mmol) was suspended in THF (20 mL) and then cooled to −78° C. A 1.8 M solution of phenyllithium in di-n-butylether (3.18 mL, 5.73 mmol) was added dropwise. After complete addition, the reaction mixture was stirred at −78° C. for 2 h. Next, a 2.5 M solution of n-BuLi in hexanes (4.77 mL, 11.93 mmol) was added dropwise. After complete addition, the reaction mixture was stirred at −78° C. for 1 h. Trimethyl borate (2.67 mL, 23.87 mmol) was then added all at once. The cold bath was removed and the thick mixture was stirred at RT for 2 h. The mixture was re-cooled to 0° C. and water (6 mL) was added carefully, followed by 6 N HCl (6 mL, 36.00 mmol). The ice bath was then removed and the mixture was stirred at RT for 1 h and then placed in the refrigerator overnight. The aq and THF layers were separated and the THF layer was discarded. The aq layer was cooled to 0° C. and aq NaOH was added until the pH was approx. 5-6. The aq layer was extracted with several portions of EtOAc. The EtOAc extracts were conc in vacuo to give a solid. The solid was then treated with aq NaOH until the pH was >9. After diluting with MTBE, the aq and MTBE layers were separated. The aq layer was washed with several additional portions of MTBE. The MTBE layers were discarded. The aq layer was then cooled to 0° C. and treated with aq HCl until the pH was approx. 5-6. The aq layer was extracted with several portions of EtOAc. The EtOAc extracts were combined and conc in vacuo to give 0.331 g (38%) of the title compound which was used without further purification.

LC/MS (ES⁺)[(M+H)⁺]: 370 for C₁₆H₁₆BN₅O₃S.

### Intermediate 175

### 4-Bromopicolinohydrazide

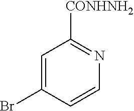

Methyl 4-bromopicolinate (1.080 g, 5.00 mmol) was dissolved in EtOH (25.00 ml). Hydrazine hydrate (2.432 ml, 50.00 mmol) was added and the mixture was heated at 85° C. for 1 h. After cooling to RT, the mixture was conc in vacuo to give the title compound which was used without further purification.

LC/MS (ES⁺)[(M+H)⁺]: 217 for C₆H₆BrN₃O

¹H NMR (DMSO-d₆): δ 10.03 (s, 1H); 8.50 (d, 1H); 8.12 (d, 1H); 7.87 (dd, 1H); 4.61 (br s, 2H).

### Intermediate 176

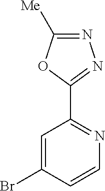

The 4-bromopicolinohydrazide (Intermediate 175 1.080 g, 5 mmol) was suspended in trimethyl orthoacetate (10 ml, 79.57 mmol). Using a pipet, 2 drops of conc HCl were added. The mixture was heated to 115° C. for 1 h and then cooled to RT. After conc in vacuo, the resultant solid was treated with additional trimethyl orthoacetate (10 ml, 79.57 mmol) and 1,8-diazabicyclo[5.4.0]undec-7-ene (1.6 mL) and heated at 110° C. for 48 h. After cooling to RT and conc in vacuo, the residue was diluted with EtOAc and washed with several portions of satd NH₄Cl until the washes were colorless. The organic layer was then washed with water, brine, dried over Na₂SO₄ and conc in vacuo. Purification by silica gel chromatography (0-10% MeOH/CH₂Cl₂) gave 0.524 g (39%) of the title compound.

LC/MS (ES⁺)[(M+H)⁺]: 241 for C₈H₆BrN₃O

¹H NMR (DMSO-d₆): δ 8.65 (d, 1H); 8.33 (d, 1H); 7.93 (dd, 1H); 2.62 (s, 3H).

### Intermediate 177

### Methyl 6-(3-ethylureido)-4-(4-(pyridin-2-yl)thiazol-2-yl)-3,4′-bipyridine-2′-carboxylate

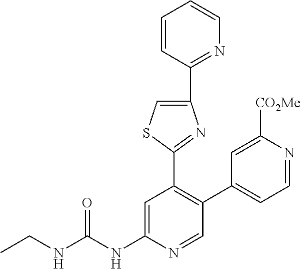

The 6-(3-ethylureido)-4-(4-(pyridin-2-yl)thiazol-2-yl)pyridin-3-ylboronic acid (Intermediate 174, 0.166 g, 0.45 mmol), methyl 4-bromopicolinate (0.117 g, 0.54 mmol), tetrakis (triphenylphosphine)palladium (0) (0.052 g, 0.05 mmol) and potassium carbonate (0.187 g, 1.35 mmol) were placed in a microwave vessel. The vessel was degassed and purged with N₂ several times. DMF (3 mL) was added and the vessel was degassed and purged with N₂ again. The vessel was heated in the microwave at 95° C. for 2 h. The mixture was filtered through a fitted funnel fitted with filter paper and rinsed with several portions of CH₂Cl₂ and once with EtOAc. The filtrate was then conc in vacuo. Purification by silica gel chromatography (0-10% MeOH/CH₂Cl₂) gave 0.037 g (18%) of the title compound.

LC/MS (ES⁺)[(M+H)⁺]: 461 for C₂₃H₂₀N₆O₃S

¹H NMR (DMSO-d₆): δ 9.52 (s, 1H); 8.73 (d, 1H); 8.60 (m, 1H); 8.38 (s, 1H); 8.34 (s, 1H); 8.22 (s, 1H); 8.02 (s, 1H); 7.82 (m, 1H); 7.60 (m, 3H); 7.35 (m, 1H); 3.84 (s, 3H); 3.22 (m, 2H); 1.11 (t, 3H).

### Intermediate 178

### 1-Ethyl-3-(2′-(hydrazinecarbonyl)-4-(4-(pyridin-2-yl)thiazol-2-yl)-3,4′-bipyridin-6-yl)urea

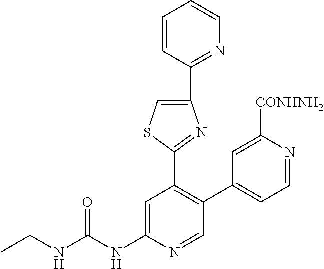

The methyl 6-(3-ethylureido)-4-(4-(pyridin-2-yl)thiazol-2-yl)-3,4′-bipyridine-2′-carboxylate (Intermediate 177, 56.4 mg, 0.12 mmol), EtOH (1.75 mL), and hydrazine hydrate (0.060 mL, 1.22 mmol) were combined and heated at 85° C. for 1 h. After cooling to RT, the mixture was conc in vacuo to give the title compound which was used without further purification.

LC/MS (ES⁺)[(M+H)⁺]: 461 for C₂₂H₂₀N₈O₂S

### Intermediate 179

### 1-(4-Chloropyridin-2-yl)-3-ethylurea

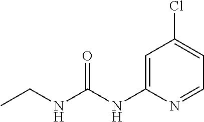

A suspension of 4-chloropyridin-2-amine (2.186 g, 17 mmol), ethyl isocyanate (2.69 mL, 34.00 mmol) and chloroform (8 mL) was heated in the microwave at 100° C. for 1 h. The resultant solution was then concentrated in vacuo to give the title compound in quantitative yield. No further purification was performed.

LC/MS (ES⁺): 200, 202 for C₈H₁₀ClN₃O

¹H NMR (DMSO-d₆): δ 9.31 (s, 1H); 8.16 (d, 1H); 7.63 (m, 1H); 7.59 (m, 1H); 7.03 (m, 1H); 3.16 (m, 2H); 1.07 (t, 3H).

### Intermediate 180

### 1-(5-Bromo-4-chloropyridin-2-yl)-3-ethylurea

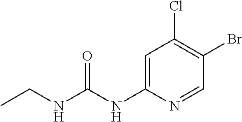

A solution of 1-(4-chloropyridin-2-yl)-3-ethylurea (Intermediate 179, 3.39 g, 16.98 mmol), N-bromosuccinimide (3.02 g, 16.98 mmol), acetonitrile (32 mL), and DMF (10 mL) were combined and heated at 80° C. for 2 h. Upon cooling to RT, a precipitate formed. Water was added and the solid was collected and washed with water to give 2.78 g (59%) of the title compound which was used without further purification.

LC/MS (ES⁺): 278, 280 for C₈H₉BrClN₃O

¹H NMR (DMSO-d₆): δ 9.37 (s, 1H); 8.44 (s, 1H); 7.92 (s, 1H); 7.17 (m, 1H); 3.15 (m, 2H); 1.06 (t, 3H).

### Intermediate 181

### Ethyl 4′-chloro-6′-(3-ethylureido)-3,3′-bipyridine-5-carboxylate

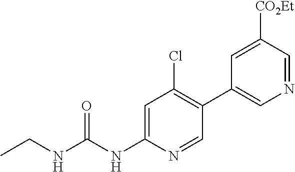

The 1-(5-bromo-4-chloropyridin-2-yl)-3-ethylurea (Intermediate 180, 0.404 g, 1.45 mmol), ethyl 5-(4,4,5,5-tetramethyl-1,3,2-dioxaborolan-2-yl)nicotinate (0.482 g, 1.74 mmol) and cesium carbonate (0.945 g, 2.90 mmol) were added to a microwave vessel. The vessel was degassed and purged with N₂. Tetrakis(triphenylphosphine)palladium (0) (0.168 g, 0.15 mmol) was added and the vessel was degassed and purged with N₂. Dioxane (10 mL) and water (2.5 mL) were added and the vessel was degassed and purged with N₂ three more times. The vessel was placed in the microwave and heated at 100° C. for 2 h. The organic layer was separated and conc in vacuo. After purification by silica gel chromatography (0-10% MeOH/CH₂Cl₂), the resultant solid was triturated with hot acetonitrile to give 0.339 g (67%) of the title compound.

LC/MS (ES⁺): 349, 351 for C₁₆H₁₇ClN₄O₃

¹H NMR (DMSO-d₆): δ 9.47 (s, 1H); 9.12 (m, 1H); 8.92 (m, 1H); 8.37 (m, 1H); 8.33 (s, 1H); 7.85 (s, 1H); 7.46 (m, 1H); 4.38 (q, 2H); 3.18 (m, 2H); 1.35 (t, 3H); 1.09 (t, 3H).

### Intermediate 182

### 1-(4-Chloro-5′-(hydrazinecarbonyl)-3,3′-bipyridin-6-yl)-3-ethylurea

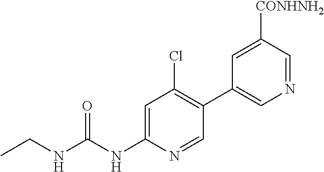

The ethyl 4′-chloro-6′-(3-ethylureido)-3,3′-bipyridine-5-carboxylate (Intermediate 181, 0.234 g, 0.67 mmol), anhydrous hydrazine (0.211 ml, 6.71 mmol) and EtOH (10 ml) were heated at 80° C. overnight. Hydrazine hydrate (0.326 ml, 6.71 mmol) was then added and the mixture was heated at 80° C. for an additional 3 h. After cooling to RT, the mixture was diluted with MeOH and concentrated in vacuo to give the title compound which was used without further purification.

LC/MS (ES⁺)[(M+H)⁺]: 335 for C₁₄H₁₅ClN₆O₂

¹H NMR (DMSO-d₆): δ 10.03 (s, 1H); 9.46 (s, 1H); 9.00 (m, 1H); 8.80 (m, 1H); 8.33 (s, 1H); 8.26 (s, 1H); 7.85 (s, 1H); 7.45 (m, 1H); 4.60 (br s, 2H); 3.18 (m, 2H); 1.09 (t, 3H).

### Intermediate 183

### 1-(4-Chloro-5′-(5-oxo-4,5-dihydro-1,3,4-oxadiazol-2-yl)-3,3′-bipyridin-6-yl)-3-ethylurea

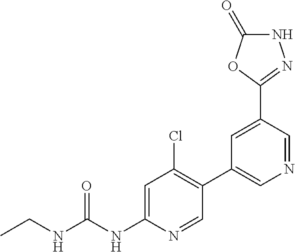

Diisopropylethylamine (0.176 ml, 1.01 mmol) was added to a solution of 1-(4-chloro-5′-(hydrazinecarbonyl)-3,3′-bipyridin-6-yl)-3-ethylurea (Intermediate 182, 0.225 g, 0.67 mmol) in DMF (6 ml). 1,1′-Carbonyldiimidazole (0.163 g, 1.01 mmol) was added in one portion and the resultant mixture was stirred at RT overnight. Water was added and the mixture was concentrated in vacuo. Purification by silica gel chromatography (0-10% MeOH/CH₂Cl₂) gave the title compound.

LC/MS (ES⁺): 361, 363 for C₁₅H₁₃ClN₆O₃

¹H NMR (DMSO-d₆): δ 12.83 (br s, 1H); 9.48 (s, 1H); 9.01 (m, 1H); 8.85 (m, 1H); 8.34 (s, 1H); 8.25 (m, 1H); 7.86 (s, 1H); 7.46 (m, 1H); 3.21 (m, 2H); 1.09 (t, 3H).

### Intermediate 184

### Ethyl 6′-(3-ethylureido)-4′-(4-morpholinophenyl)-3,3′-bipyridine-5-carboxylate

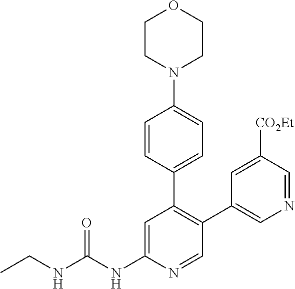

Following the procedure for Example 107, ethyl 4′-chloro-6′-(3-ethylureido)-3,3′-bipyridine-5-carboxylate (Intermediate 181, 0.315 g, 0.90 mmol) and 4-morpholinophenylboronic acid (0.251 g, 1.21 mmol) were heated in the microwave for 1 h at 100° C. After conc in vacuo, CH₂Cl₂ and water were added and the layers were separated. The organic layer was concentrated in vacuo and then purified by silica gel chromatography (0-100% EtOAc/hexanes) to give 0.268 g (62%) of the title compound.

LC/MS (ES⁺)[(M+H)⁺]: 476 for C₂₆H₂₉N₅O₄

¹H NMR (DMSO-d₆): δ 9.36 (s, 1H); 8.94 (m, 1H); 8.50 (m, 1H); 8.26 (s, 1H); 8.00 (m, 2H); 7.48 (s, 1H); 6.98 (m, 2H); 6.88 (m, 2H); 4.31 (q, 2H); 3.71 (m, 4H); 3.21 (m, 2H); 3.11 (m, 4H); 1.29 (t, 3H); 1.10 (t, 3H).

### Intermediate 185

### 1-Ethyl-3-(5′-(hydrazinecarbonyl)-4-(4-morpholinophenyl)-3,3′-bipyridin-6-yl)urea

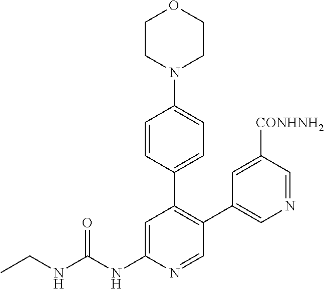

Hydrazine hydrate (0.215 mL, 4.42 mmol) was added to a suspension of ethyl 6′-(3-ethylureido)-4′-(4-morpholinophenyl)-3,3′-bipyridine-5-carboxylate (Intermediate 184, 0.105 g, 0.22 mmol) in EtOH (3 mL). The mixture was heated at 80° C. overnight. After cooling to RT, the mixture was diluted with MeOH and concentrated in vacuo to give the title compound which was used without further purification.

LC/MS (ES⁺)[(M+H)⁺]: 462 for C₂₄H₂₇N₇O₃

¹H NMR (DMSO-d₆): δ 9.93 (br s, 1H); 9.34 (s, 1H); 8.81 (m, 1H); 8.29 (m, 1H); 8.23 (s, 1H); 8.03 (m, 1H); 7.96 (m, 1H); 7.48 (s, 1H); 6.98 (m, 2H); 6.88 (m, 2H); 4.55 (br s, 2H); 3.70 (m, 4H); 3.20 (m, 2H); 3.11 (m, 4H); 1.10 (t, 3H).

### Intermediate 186

### 5-bromo-6-hydroxypyridine-3-carboxylic acid

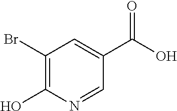

To a stirred suspension of 6-hydroxypyridine-3-carboxylic acid (13.0 g, 215 mmol) in water (150 mL) was added bromine (16 mL, 310 mmol) dropwise slowly at 0° C. over a period of 30 min. The reaction mixture was stirred at 0° C. for 30 min and slowly the temperature was allowed to rise to room temperature. The reaction mixture was stirred at room temperature for 4 h. The reaction mixture was treated with saturated sodium metabisulphite solution and stirred for another 30 min at room temperature. The precipitated product was collected by filtration and washed with excess water and dried to afford 35 g (70%) of 5-bromo-6-hydroxypyridine-3-carboxylic acid as an off-white solid.

¹H NMR (400 MHz, DMSO-d₆) δ 8.03 (s, 1H), 8.16 (s, 1H), 12.58 (br s, 1H).

Mass: m/z 218, 220 (M, M+2)

### Intermediate 187

### Methyl 5-bromo-6-hydroxypyridine-3-carboxylate

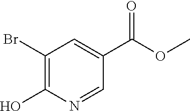

To a stirred solution of 5-bromo-6-hydroxypyridine-3-carboxylic acid (Intermediate 186, 10 g, 45.87 mmol) in methanol (100 mL) was added sulphuric acid (1 mL) and the reaction mixture was heated to reflux overnight. The solvent was concentrated under reduced pressure to get crude compound which was poured into saturated sodium bicarbonate solution. The solid that formed was collected by filtration and dried to afford 8.5 g (80%) of methyl 5-bromo-6-hydroxypyridine-3-carboxylate.

¹H NMR (400 MHz, DMSO-d₆) δ 3.78 (s, 3H), 8.10 (s, 1H), 8.18 (s, 1H), 12.71 (br s, 1H).

MASS (ES): m/z 234 (M+H).

### Intermediate 188

### Methyl 5-bromo-6-{[1-(tert-butoxycarbonyl)piperidin-4-yl]oxy}pyridine-3-carboxylate

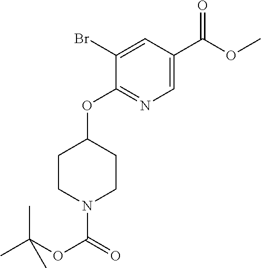

To a stirred solution of methyl 5-bromo-6-hydroxypyridine-3-carboxylate (Intermediate 187, 4.0 g, 17.24 mmol) in dry tetrahydrofuran (50 mL), was added tert-butyl 4-hydroxypiperidine-1-carboxylate (3.46 g, 17.24 mmol), and triphenylphosphine (13.42 g, 51.22 mmol) at 0° C. The reaction mixture was stirred for 10 min followed by addition of diethyl azodicarboxylate (4.0 g, 22.9 mmol). The reaction mixture was maintained at room temperature and stirred for 3 h. The solvent was concentrated under reduced pressure, then water was added and the product was extracted into ethyl acetate (2×50 mL, 1×25 mL). The combined organic layers were dried over anhydrous sodium sulphate, filtered then concentrated under reduce pressure to obtain crude compound which was purified by flash column chromatography (25-30% ethyl acetate/pet ether) to afford 5.0 g (70%) of methyl 5-bromo-6-{[1-(tert-butoxycarbonyl)piperidin-4-yl]oxy}pyridine-3-carboxylate

¹H NMR (400 MHz, DMSO-d₆): δ 1.45 (s, 9H), 1.85 (m, 2H), 1.95 (m, 2H), 3.48 (m, 2H), 3.65 (m, 2H), 3.91 (s, 3H), 5.39 (m, 1H), 8.39 (s, 1H), 8.70 (s, 1H).

MASS (APCI): m/z 417 (M+2).

### Intermediate 189

### Methyl 2-{[1-(tert-butoxycarbonyl)piperidin-4-yl]oxy}-6′-[(ethylcarbamoyl)amino]-4′-[4-(trifluoromethyl)-1,3-thiazol-2-yl]-3,3′-bipyridine-5-carboxylate

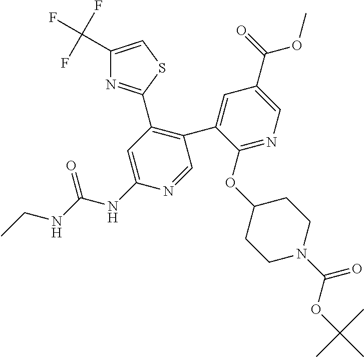

In a round bottomed flask methyl 5-bromo-6-{[1-(tert-butoxycarbonyl)piperidin-4-yl]oxy}pyridine-3-carboxylate (Intermediate 188, 300 mg, 0.72 mmol), 1-ethyl-3-{5-(4,4,5,5-tetramethyl-1,3,2-dioxaborolan-2-yl)-4-[4-(trifluoromethyl)-1,3-thiazol-2-yl]pyridin-2-yl}urea (Intermediate 12, 351 mg, 8.31 mmol) and cesium carbonate (470 mg, 1.44 mmol) were suspended in 1,4 dioxane:water (8:2) (25 mL). This reaction mixture was purged with Argon gas for 30 min. Tetrakis(triphenylphosphine)palladium (167 mg, 0.14 mmol) was added under argon atmosphere and the reaction mixture was heated to 80-90° C. for 3 h. The reaction mixture was cooled to room temperature, filtered through celite, the filtrate was concentrated under reduced pressure to obtain a residue which was purified by flash column chromatography (20-25% ethyl acetate/pet ether) to afford 0.25 g (56.8%) of methyl 2-{[1-(tert-butoxycarbonyl)piperidin-4-yl]oxy}-6′-[(ethylcarbamoyl)amino]-4′-[4-(trifluoromethyl)-1,3-thiazol-2-yl]-3,3′-bipyridine-5-carboxylate.

¹H NMR (400 MHz, DMSO-d₆): δ 1.08 (t, 3H), 1.35 (s, 9H), 1.55 (d, 2H), 3.05 (m, 3H), 3.20 (m, 6H), 3.87 (s, 3H), 5.09 (m, 1H), 7.60 (br s, 1H), 8.20 (s, 1H), 8.25 (s, 1H), 8.50 (s, 1H), 8.79 (s, 1H), 9.46 (br s, 1H).

MASS (APCI): m/z 651.1 (M+H).

### Intermediate 190

### Tert-butyl 4-({6′-[(tethylcarbamoyl)amino]-5-(hydrazinylcarbonyl)-4′-[4-(trifluoromethyl)-1,3-thiazol-2-yl]-3,3′-bipyridin-2-yl}oxy)piperidine-1-carboxylate

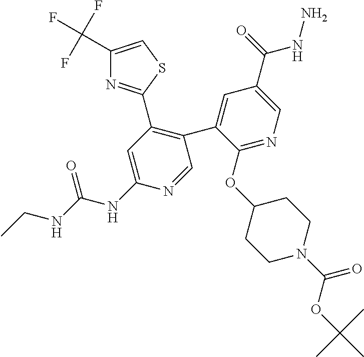

To a stirred solution of methyl 2-{[1-(tert-butoxycarbonyl)piperidin-4-yl]oxy}-6′-[(ethylcarbamoyl)amino]-4′-[4-(trifluoromethyl)-1,3-thiazol-2-yl]-3,3′-bipyridine-5-carboxylate (Intermediate 189, 0.5 g, 0.76 mmol) in ethanol (20 mL) was added hydrazine hydrate (2.0 mL, 40 mmol) and the resulting mixture was heated to reflux temperature for 4 h. The reaction mixture was cooled, the solvent was concentrated under reduced pressure. Diethyl ether (10 mL) was added, and the mixture was stirred for 10 min. The resulting solid was collected by filtration and dried to afford 0.4 g (80%) of tert-butyl 4-({6′-[(ethylcarbamoyl)amino]-5-(hydrazinylcarbonyl)-4′-[4-(trifluoromethyl)-1,3-thiazol-2-yl]-3,3′-bipyridin-2-yl}oxy)piperidine-1-carboxylate as solid.

MASS (APCI): m/z 651.1 (M+H).

### Intermediate 191

### Tert-butyl 4-({6′-[(ethylcarbamoyl)amino]-5-(5-oxo-4,5-dihydro-1,3,4-oxadiazol-2-yl)-4′-[4-(trifluoromethyl)-1,3-thiazol-2-yl]-3,3′-bipyridin-2-yl}oxy)piperidine-1-carboxylate

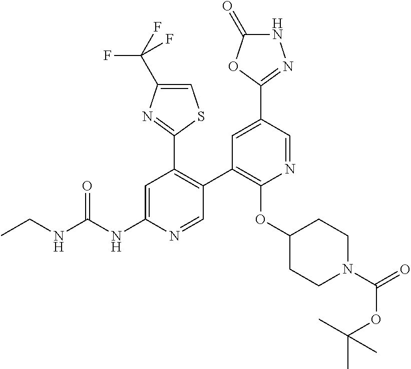

To a stirred solution of tert-butyl 4-({6′-[(ethylcarbamoyl)amino]-5-(hydrazinylcarbonyl)-4′-[4-(trifluoromethyl)-1,3-thiazol-2-yl]-3,3′-bipyridin-2-yl}oxy)piperidine-1-carboxylate (Intermediate 190, 300 mg, 0.46 mmol) in tetrahydrofuran (15 mL), phosgene (0.34 mL in toluene, 0.67 mmol) was slowly added at 0° C. The reaction mixture was stirred at room temperature which for 3 h. The solvent was concentrated under reduced pressure and the resulting residue was purified by flash column chromatography (5-10% ethyl acetate/pet ether) to afford 0.2 g (64.9%) of tert-butyl 4-({6′-[(ethylcarbamoyl)amino]-5-(5-oxo-4,5-dihydro-1,3,4-oxadiazol-2-yl)-4′-[4-(trifluoromethyl)-1,3-thiazol-2-yl]-3,3′-bipyridin-2-yl}oxy)piperidine-1-carboxylate.

¹H NMR (400 MHz, CDCl₃): δ 1.25 (m, 6H), 1.40 (s, 9H), 3.11 (m, 2H), 3.42 (br s, 2H), 3.51 (m 2H), 3.94 (s, 3H), 5.13 (m, 1H), 7.53 (s, 1H), 7.70 (s, 1H), 8.13 (s, 1H), 8.21 (d, 1H), 8.81 (br s, 1H), 9.04 (m, 1H).

LC-MS: m/z 677.0 (M+2).

### Intermediate 192

### Tert-butyl 4-{6′-[(ethylcarbamoyl)amino]-5-(5-methyl-1,3,4-oxadiazol-2-yl)-4′-[4-(trifluoromethyl)-1,3-thiazol-2-yl]-3,3′-bipyridin-2-yl}oxy)piperidine-1-carboxylate

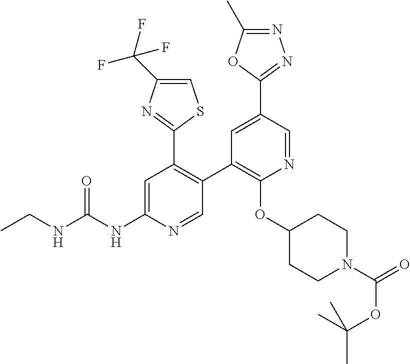

tert-Butyl 4-({6′-[(ethylcarbamoyl)amino]-5-(hydrazinylcarbonyl)-4′-[4-(trifluoromethyl)-1,3-thiazol-2-yl]-3,3′-bipyridin-2-yl}oxy)piperidine-1-carboxylate (Intermediate 190, 200 mg, 0.30 mmol) was dissolved in triethylorthoacetate (5 mL) and the reaction mixture was heated to 120° C. for 12 h. The reaction mixture was cooled to room temperature, diluted with ethyl acetate and the organics were extracted into ethyl acetate. The combined organic layers were washed with water and brine, dried over anhydrous sodium sulphate and evaporated under reduced pressure to obtain crude compound which was purified by flash column chromatography (25-35% ethyl acetate/pet ether) to afford 100 mg 48.3% tert-butyl 4-({6′-[(ethylcarbamoyl)amino]-5-(5-methyl-1,3,4-oxadiazol-2-yl)-4′-[4-(trifluoromethyl)-1,3-thiazol-2-yl]-3,3′-bipyridin-2-yl}oxy)piperidine-1-carboxylate as solid.

¹H NMR (400 MHz, CDCl₃): δ 0.89 (t, 3H), 1.48 (S, 9H), 2.52 (s, 3H), 3.18 (m, 2H), 3.38-3.46 (m, 4H), 5.18 (m, 1H), 7.42-7.56 (m, 7H), 8.18-8.24 (m, 2H), 8.82 (s, 1H), 9.02 (br s, 1H)

MASS (APCI): m/z 674.2 (M−H).

### Intermediate 193

### Methyl 5-bromo-6-(3-tert-butoxy-3-oxopropoxy)pyridine-3-carboxylate

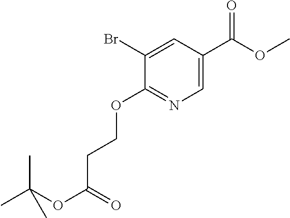

To a stirred solution of methyl 5-bromo-6-hydroxypyridine-3-carboxylate (Intermediate 187, 1.0 g, 4.31 mmol) in dry tetrahydrofuran (50 mL), tert-butyl 3-hydroxypropanoate (1.26 g, 8.62 mmol) and triphenylphosphine (2.25 g, 8.62 mmol) were added and stirred for 10 min. Then the reaction mixture was cooled to 0° C., diethyl azodicarboxylate (1.5 g, 8.62 mmol) was added slowly. The reaction mixture was maintained at room temperature for 4 h. After the completion of the reaction, the solvent was concentrated under reduced pressure; water was added and the product was extracted into ethyl acetate (2×50 mL, 1×25 mL). The combined organic layers were dried over anhydrous sodium sulphate and evaporated under reduced pressure to yield the crude product which was purified by flash column chromatography (5-10% ethyl acetate/pet ether) to afford 700 mg (46%) of methyl 5-bromo-6-(3-tert-butoxy-3-oxopropoxy)pyridine-3-carboxylate.

¹H NMR (400 MHz, CDCl₃): δ 1.41 (s, 9H), 2.78 (t, 2H), 3.87 (s, 3H), 4.27 (t, 2H), 8.26 (s, 1H), 8.31 (s, 1H).

LC MS: m/z 360.1 (M+H).

### Intermediate 194

### Methyl 2-(3-tert-butoxy-3-oxopropoxy)-6′-[(ethylcarbamoyl)amino]-4′-[4-(trifluoromethyl)-1,3-thiazol-2-yl]-3,3′-bipyridine-5-carboxylate

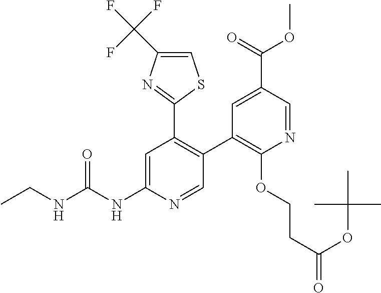

In a round bottomed flask methyl 5-bromo-6-(3-tert-butoxy-3-oxopropoxy)pyridine-3-carboxylate (Intermediate 193, 10.7 g, 1.94 mmol), 1-ethyl-3-{5-(4,4,5,5-tetramethyl-1,3,2-dioxaborolan-2-yl)-4-[4-(trifluoromethyl)-1,3-thiazol-2-yl]pyridin-2-yl}urea (Intermediate 12, 0.94 g, 2.13 mmol) and cesium carbonate (1.26 g, 3.88 mmol), were suspended in 1,4 dioxane: water (10 mL) (1:4) The above mixture was purged with Argon gas for 30 min. Tetrakis(triphenylphosphine)palladium (0.44 g, 0.38 mM) was added under argon atmosphere and the reaction mixture was heated to 100° C. for 4 h. After the completion of the reaction, the reaction mixture was cooled to room temperature, filtered through celite, the organic solvent was concentrated under reduced pressure to get a residue which was purified by flash column chromatography (gradient up to 40% ethyl acetate in pet ether) to afford 350 mg (30%) of methyl 2-(3-tert-butoxy-3-oxopropoxy)-6′-[(ethylcarbamoyl)amino]-4′-[4-(trifluoromethyl)-1,3-thiazol-2-yl]-3,3′-bipyridine-5-carboxylate.

LC-MS: m/z 595 (M+H).

### Intermediate 195

### 3-({6′-[(ethylcarbamoyl)amino]-5-methoxycarbonyl)-4′-[4-trifluoromethyl)-1,3-thiazol-2-yl]-3,3′-bipyridin-2-yl}oxy)propanoic acid

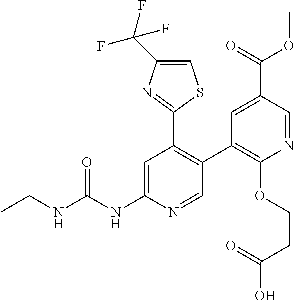

To a solution methyl 2-(3-tert-butoxy-3-oxopropoxy)-6′-[(ethylcarbamoyl)amino]-4′-[4-(trifluoromethyl)-1,3-thiazol-2-yl]-3,3′-bipyridine-5-carboxylate (Intermediate 194, 350 mg, 0.58 mmol) in dichloromethane (20 mL), trifluoroacetic acid (335 mg, 2.94 mmol) was added and the mixture was stirred for 6 h at room temperature. Volatiles were evaporated under reduced pressure to afford the crude product, which was purified by flash column chromatography (gradient up to 5% methanol in chloroform) to afford 300 mg (96%) 3-({6′-[(ethylcarbamoyl)amino]-5-(methoxycarbonyl)-4′-[4-(trifluoromethyl)-1,3-thiazol-2-yl]-3,3′-bipyridin-2-yl}oxy)propanoic acid.

¹H NMR (400 MHz, DMSO-d₆): δ 1.21 (t, 1H), 1.25 (m, 2H), 1.85 (s, 2H), 2.35 (s, 2H), 3.30 (m, 2H), 3.89 (s, 3H), 4.00 (t, 2H), 7.90 (s, 1H), 8.15 (d, 2H), 8.50 (s, 1H), 8.63 (s, 1H), 9.39 (s, 1H).

LC-MS: m/z 540.3 (M+H).

### Intermediate 196

### 3-({6′-[(ethylcarbamoyl)amino]-5-(hydrazinylcarbonyl)-4′-[4-trifluoromethyl)-1,3-thiazol-2-yl]-3,3′-bipyridin-2-yl}oxy)propanoic acid

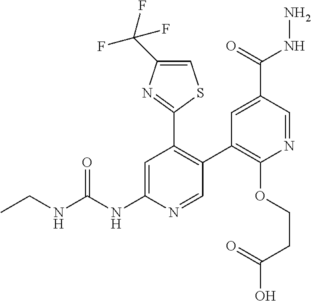

To a stirred solution of 3-({6′-[(ethylcarbamoyl)amino]-5-(methoxycarbonyl)-4′-[4-(trifluoromethyl)-1,3-thiazol-2-yl]-3,3′-bipyridin-2-yl}oxy)propanoic acid (Intermediate 195, 300 mg, 0.55 mmol) in ethanol (20 mL), hydrazine hydrate (1.28 g, 25.6 mmol) was added at room temperature and the resulting reaction mixture was heated to reflux for 3 h. The reaction mixture was cooled, and the solvent was concentrated under reduced pressure. Diethyl ether (10 mL) was added and the mixture was stirred for 10 min. The obtained solid was filtered and dried to afford 240 mg (80%) of 3-({6′-[(ethylcarbamoyl)amino]-5-(hydrazinylcarbonyl)-4′-[4-(trifluoromethyl)-1,3-thiazol-2-yl]-3,3′-bipyridin-2-yl}oxy)propanoic acid.

¹H NMR (400 MHz, DMSO-d₆): δ 1.12 (t, 3H), 2.35 (br s, 2H), 3.22 (t, 2H), 4.01 (m, 2H), 7.65 (br s, 1H), 8.10 (s, 1H), 8.20 (d, 2H), 8.50 (d, 2H), 9.40 (s, 1H), 9.50 (br s, 1H).

### Intermediate 197

### Methyl 5-bromo-6-(tetrahydro-2H-pyran-4-ylmethoxy)pyridine-3-carboxylate

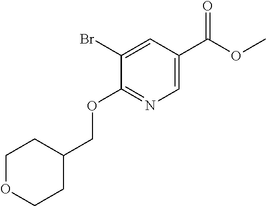

To a stirred solution of methyl 5-bromo-6-hydroxypyridine-3-carboxylate (Intermediate 187, 2.0 g, 8.62 mmol) in tetrahydrofuran (50 mL), was added tetrahydro-2H-pyran-4-ylmethanol (2.0 g, 17.24 mmol), triphenylphosphine (4.51 g, 17.24 mmol) stirred for 10 min. Then the reaction mixture was cooled to 0° C. Diethyl azodicarboxylate (3.0 g, 17.24 mmol) was added slowly, and the reaction mixture was maintained at room temperature for 3 h. The solvent was concentrated under reduced pressure. Water was added and the product was extracted into ethyl acetate (2×50 mL, 1×25 mL). The combined organic layers were dried over anhydrous sodium sulphate and evaporated under reduced pressure to yield the crude product which was purified by flash column chromatography (product eluted with 5-10% ethyl acetate/pet ether) to afford 2.0 g (71%) of methyl 5-bromo-6-(tetrahydro-2H-pyran-4-ylmethoxy)pyridine-3-carboxylate.

¹H NMR (400 MHz, CDCl₃): δ 1.51 (m, 2H), 1.78 (d, 2H), 2.10 (m, 1H), 3.45 (t, 2H), 3.91 (s, 3H), 4.28 (d, 2H), 8.39 (s, 1H), 8.71 (s, 1H).

MASS: m/z 329.8 (M+H).

### Intermediate 198

### Methyl 6′-[(ethylcarbamoyl)amino]-2-(tetrahydro-2H-pyran-4-ylmethoxy)-4′-[4-(trifluoromethyl)-1,3-thiazol-2-yl]-3,3′-bipyridine-5-carboxylate

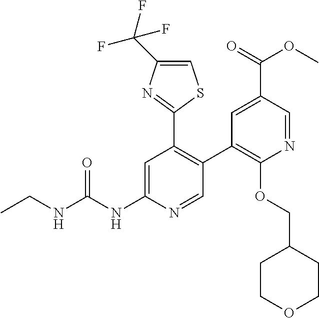

In a round bottomed flask methyl 5-bromo-6-(tetrahydro-2H-pyran-4-ylmethoxy)pyridine-3-carboxylate (Intermediate 197, 1.5 g, 4.54 mmol), 1-ethyl-3-{5-(4,4,5,5-tetramethyl-1,3,2-dioxaborolan-2-yl)-4-[4-(trifluoromethyl)-1,3-thiazol-2-yl]pyridin-2-yl}urea (Intermediate 12, 2.22 g, 5.02 mmol) and sodium bicarbonate (0.76 g, 9.04 mmol) which was dissolved in minimum amount of water (10 mL), were combined and suspended in toluene:water (1:4). The reaction mixture was purged with argon gas for 30 min. Tetrakis(triphenylphosphine) palladium (3.31 g, 0.268 mmol) was added under argon atmosphere and the reaction mixture was heated to 80-90° C. for 5 h. The reaction mixture was cooled to room temperature, filtered through celite bed; the organic solvent was concentrated under reduced pressure to yield a residue. To this residue acetonitrile was added to obtain solid which was filtered and dried to afford 1.2 g (46%) of methyl 6′-[(ethylcarbamoyl)amino]-2-(tetrahydro-2H-pyran-4-ylmethoxy)-4′-[4-(trifluoromethyl)-1,3-thiazol-2-yl]-3,3′-bipyridine-5-carboxylate.

¹H NMR (400 MHz, CDCl₃) δ 1.09 (m, 3H), 1.29 (t, 5H), 1.65 (m, 1H), 3.25 (t, 2H), 3.50 (m, 2H), 3.91 (dd, 2H), 3.95 (s, 3H), 7.51 (m, 2H), 7.71 (s, 2H), 7.95 (br s, 1H), 8.15 (s, 1H), 8.25 (d, 1H), 8.95 (d 1H)

LC-MS: m/z 566.5 (M+H).

### Intermediate 199

### 1-Ethyl-3-{5′-(hydrazinylcarbonyl)-2′-(tetrahydro-2H-pyran-4-ylmethoxy)-4-[4-(trifluoromethyl)-1,3-thiazol-2-yl]-3,3′-bipyridin-6-yl}urea

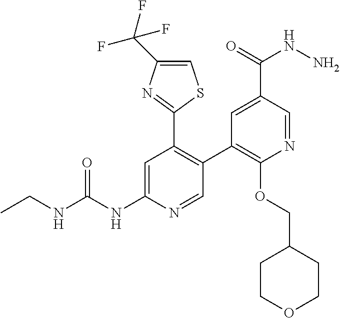

To a stirred solution of methyl 6′-[(ethylcarbamoyl)amino]-2-(tetrahydro-2H-pyran-4-ylmethoxy)-4′-[4-(trifluoromethyl)-1,3-thiazol-2-yl]-3,3′-bipyridine-5-carboxylate (Intermediate 198, 1.2 g, 2.12 mmol) in ethanol (20 mL), hydrazine hydrate (4.88 g, 97.69 mmol) was added at room temperature and the resulting reaction mixture was maintained at reflux temperature for 4 h. The reaction mixture was cooled, and the solvent was concentrated under reduced pressure to yield a residue. To this residue diethyl ether (10 mL) was added and the mixture was stirred for 10 min to obtain solid which was filtered and dried to afford 0.8 g (66%) of 1-ethyl-3-{5′-(hydrazinylcarbonyl)-2′-(tetrahydro-2H-pyran-4-ylmethoxy)-4-[4-(trifluoromethyl)-1,3-thiazol-2-yl]-3,3′-bipyridin-6-yl}urea.

¹H NMR (400 MHz, DMSO-d₆): δ 0.91 (m, 2H), 1.15 (t, 4H), 1.55 (m, 1H), 3.20 (m, 4H), 3.75 (d, 2H), 3.95 (d, 2H), 4.50 (br s, 1H), 7.60 (br s, 1H), 8.35 (d, 1H), 7.37 (d, 1H), 8.45 (s, 1H), 8.65 (s, 1H), 9.45 (s, 1H), 9.85 (br s, 1H)

MASS (APCI): m/z 564.7 (M−H).

### Intermediates 200-202

The following Intermediates were prepared according to the general procedure described below from the starting materials indicated in the Table.

### General Procedure

A suspension of methyl 6′-(3-ethylureido)-2-fluoro-4′-(4-(trifluoromethyl)thiazol-2-yl)-3,3′-bipyridine-5-carboxylate (Intermediate 162, 0.3 g, 6 mmol, 1 eq) in the appropriate alcohol (2-3 mL, ˜50 equiv) in small vial was stirred for 2 min at room temperature. Sodium hydride (0.150 g, 60 mmol) was added over 5 min and the reaction mixture was stirred for a further 5 h at room temperature. The reaction mixture was cooled with an ice bath and slowly quenched with HCl (0.1N) solution until ˜pH 7. The aqueous layer was extracted twice with ether to remove excess alcohol. The aqueous layer was concentrated in vacuo to almost dryness than loaded on Analogix C18-column for reverse phase purification (water-methanol) to remove excess of salt.

| Int | Compound | Data | SM |
|---|---|---|---|
| 200 | 2-(2-(diisopropyl- amino)ethoxy)- 6′-(3-ethylureido)-4′-(4- (trifluoromethyl)thiazol-2- yl)-3,3′-bipyridine-5- carboxylic acid 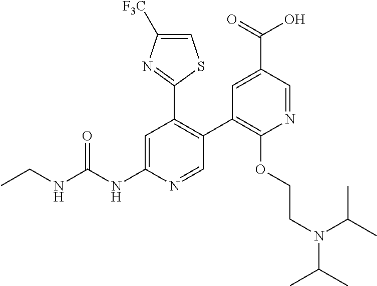 | MS (ESP): 581.21 (MH⁺) for C₂₆H₃₁F₃N₆O₄S | 2-(2-(diiso- propylamino) ethanol |
| 201 | 2-(1-(dimethylamino)propan- 2-yloxy)-6′-(3-ethylureido)- 4′-(4-(trifluoromethyl)thiazol-2- yl)-3,3′-bipyridine-5- carboxylic acid 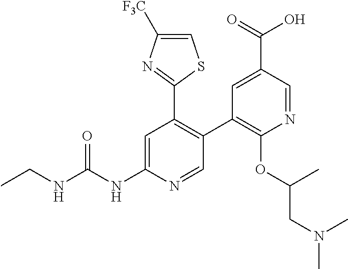 | MS (ESP): 539.16 (MH⁺) for C₂₃H₂₅F₃N₆O₄S | 2-(1-(dimethyl- amino)propan- 2-ylanol |
| 202 | 2-(2-(Diethylamino)ethoxy)- 6′-(3-ethylureido)-4′-(4- (trifluoromethyl)thiazol-2- yl)-3,3′-bipyridine-5- carboxylic acid 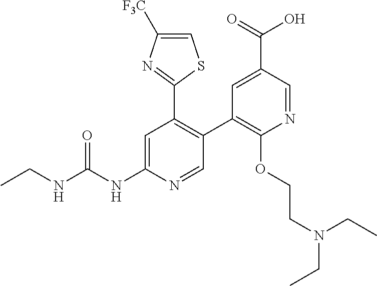 | MS (ESP): 553.18 (MH⁺) for C₂₄H₂₇F₃N₆O₄S | 2-(2-(Diethyl- amino)ethanol |

### Intermediate 203

### 6′-(3-ethylureido)-2-(1,2,2,6,6-pentamethylpiperidin-4-yloxy)-4′-(4-(trifluoromethyl)thiazol-2-yl)-3,3′-bipyridine-5-carboxylic acid

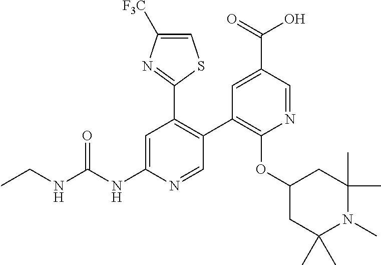

A suspension of methyl 6′-(3-ethylureido)-2-fluoro-4′-(4-(trifluoromethyl)thiazol-2-yl)-3,3′-bipyridine-5-carboxylate (Intermediate 162, 0.3 g, 6 mmol) in dimethyl formamide (3 mL) was treated with 1,2,2,6,6-pentamethylpiperidin-4-ol (5 eq) and potassium hexamethyl disilylazide (5 equivalents) in a small vial. The reaction was stirred for 48 h at room temperature. The dimethyl formamide was removed under vacuum, and the residue cooled with an ice bath and slowly quenched with HCl (0.1N) solution until pH 7. The aqueous layer was concentrated in vacuo to almost dryness than loaded on Analogix C18-column for reverse phase purification (water-methanol) to remove remaining starting materials and give an off-white solid.

MS (ESP): 606.22 (MH⁺) for C₂₈H₃₃F₃N₆O₄S

### Intermediate 204

### 2-(2-tert-butoxyethoxy)-6′-(3-ethylureido)-4′-(4-(trifluoromethyl)thiazol-2-yl)-3,3′-bipyridine-5-carboxylic acid

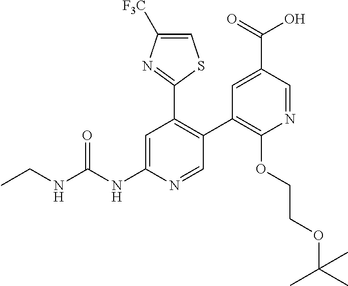

A suspension of methyl 6′-(3-ethylureido)-2-fluoro-4′-(4-(trifluoromethyl)thiazol-2-yl)-3,3′-bipyridine-5-carboxylate (Intermediate 162, 0.3 g, 6 mmol, 1 eq) in 2-tert-butoxyethanol (2-3 mL) in a small vial was stirred for 2 min at room temperature. Sodium hydride (0.150 g, 60 mmol) was added over 5 min and the reaction mixture was stirred for a further 5 h at room temperature. The reaction was cooled with an ice bath and slowly quenched with HCl (0.1N) solution to pH 7. The aqueous layer was extracted twice with ether to remove excess of alcohol. The aqueous layer was concentrated in vacuo to almost dryness than loaded on Analogix C18-column for reverse phase purification (water-methanol) to remove excess salt and give an off-white solid.

MS (ESP): 553.16 (MH⁺) for C₂₄H₂₆F₃N₅O₅S.

### Intermediate 205

### 1-(2′-(2-chloroethoxy)-5′-(5-methyl-1,3,4-oxadiazol-2-yl)-4-(4-(trifluoromethyl)thiazol-2-yl)-3,3′-bipyridin-6-yl)-3-ethylurea

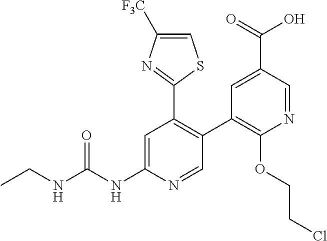

A suspension of 2-(2-tert-butoxyethoxy)-6′-(3-ethylureido)-4′-(4-(trifluoromethyl)thiazol-2-yl)-3,3′-bipyridine-5-carboxylic acid (Intermediate 204, 0.3 mmol), and hydrazine acetate (0.9 mmol) in phosphorus oxychloride (2.5 mL) was heated at 70° C. for 2 h. The solution was then cooled and concentrated to dryness. A solution of saturated potassium carbonate was added to the crude and product was extracted with ethyl acetate (3×). The combined organic layers were washed with brine and dried over sodium sulfate. All solvents were removed under vacuum and the crude was purified by Analogix using dichloromethane-methanol.

MS (ESP): 553.09 (MH⁺) for C₂₂H₁₉ClF₃N₇O₃S.

### Intermediates 206-209

The following Intermediates were synthesized according to the general procedure described below from the starting materials indicated in the Table.

### General Procedure

A suspension of corresponding carboxylic acid (0.3 mmol) in thionyl chloride (2 mL) was heated at 50° C. for 1 h. The solution was then cooled and concentrated under reduced pressure to dryness. The crude suspended in tetrahydrofuran (2 mL) was added slowly to a solution of hydrazine/tetrahydrofuran (1/2 vol., 3 mL) and stirred at room temperature for 12 h. The crude reaction mixture was concentrated to dryness and purified by reverse phase on Analogix C18-column (water-methanol) to give a (˜60%) hydrazide as an off-white solid.

| Int | Compound | Data | SM |
|---|---|---|---|
| 206 | 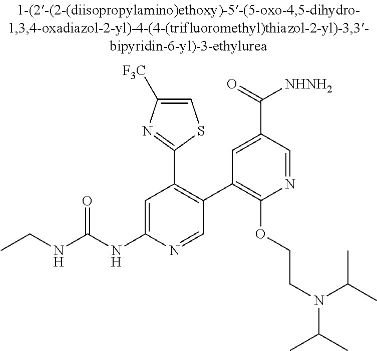 | MS (ESP): 594.23 (MH⁺) for C₂₆H₃₃F₃N₈O₃S | Intermediate 162 and 2-(2- (diisopropylamino) ethanol |
| 207 | 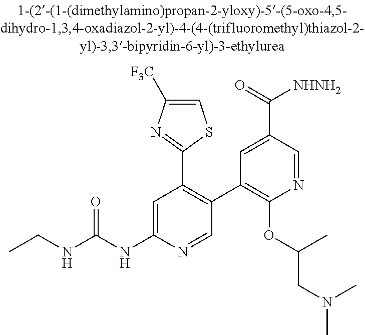 | MS (ESP): 552.19 (MH⁺) for C₂₃H₂₇F₃N₈O₃S | Intermediate 162 and 1-(2′-(1- (dimethylamino) propan-2- ol |
| 208 | 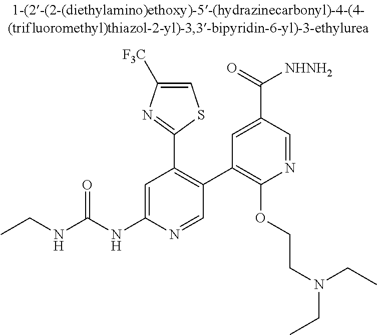 | MS (ESP): 566.20 (MH⁺) for C₂₄H₂₉F₃N₈O₃S | Intermediate 162 and 1-(2′-(2- (diethylamino)ethanol |
| 209 | 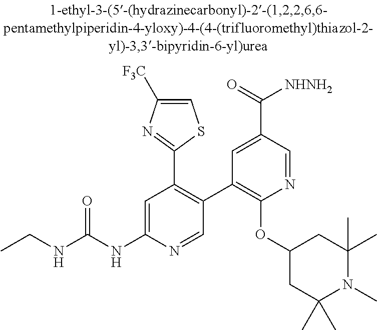 | MS (ESP): 620.25 (MH⁺) for C₂₈H₃₅F₃N₈O₃S | Intermediate 162 and 1,2,2,6,6- pentamethyl- piperidin-4-ol |

### Intermediate 210

### (S)-tert-butyl 1-hydrazinyl-3-methyl-1-oxobutan-2-ylcarbamate

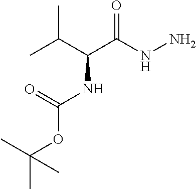

To (S)-methyl 2-(tert-butoxycarbonylamino)-3-methylbutanoate (5 g, 0.0215 mol) in ethanol (50 mL) was added hydrazine hydrate (16 mL, 0.323 mol) and the solution heated at 70° C. overnight. The solvent was evaporated and the residue dissolved in ethyl acetate, washed with water, dried over sodium sulfate and the solvent evaporated to give ˜3 g of product.

### Intermediate 211

### (S)-tert-butyl 1-(2-(2-(2-(diethylamino)ethoxy)-6′-(3-ethylureido)-4′-(4-(trifluoromethyl)thiazol-2-yl)-3,3′-bipyridine-5-carbonyl)hydrazinyl)-3-methyl-1-oxobutan-2-ylcarbamate

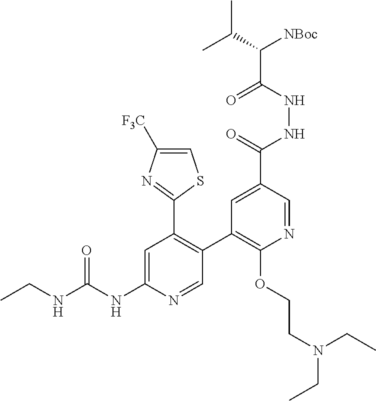

A suspension of 2-(2-(diethylamino)ethoxy)-6′-(3-ethylureido)-4′-(4-(trifluoromethyl)thiazol-2-yl)-3,3′-bipyridine-5-carboxylic acid (Intermediate 202, 0.3 mmol) in tetrahydrofuran (2 mL) was treated with (S)-tert-butyl 1-hydrazinyl-3-methyl-1-oxobutan-2-ylcarbamate (Intermediate 210, 0.6 mmol), O-(7-azabenzotriazol-1-yl)-N,N,N′,N′-tetramethyluronium hexafluorophosphate (HATU, 0.4 mmol) and triethyl amine (0.6 mmol) at room temperature for 12 h. The solution was then concentrated to dryness and purified by Analogix silica gel chromatography (dichloromethane-methanol) to give (60%) of (S)-tert-butyl 1424242-(diethylamino)ethoxy)-6′-(3-ethylureido)-4′-(4-(trifluoromethyl)thiazol-2-yl)-3,3′-bipyridine-5-carbonyl)hydrazinyl)-3-methyl-1-oxobutan-2-ylcarbamate as an off-white solid.

MS (ESP): 766 (MH⁺) for C₃₄H₄₆F₃N₉O₆S.

### Intermediate 212

### (S)-tert-butyl 1-(5-(2-(2-(diethylamino)ethoxy)-6′-(3-ethylureido)-4′-(4-(trifluoromethyl)thiazol-2-yl)-3,3′-bipyridin-5-yl)-1,3,4-oxadiazol-2-yl)-2-methylpropylcarbamate

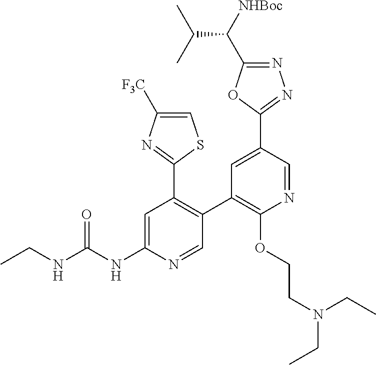

A suspension of (5)-tert-butyl 1-(2-(2-(2-(diethylamino)ethoxy)-6′-(3-ethylureido)-4′-(4-(trifluoromethyl)thiazol-2-yl)-3,3′-bipyridine-5-carbonyl)hydrazinyl)-3-methyl-1-oxobutan-2-ylcarbamate (Intermediate 211, 0.3 mmol) in tetrahydrofuran (2 mL) was treated with triphenylphosphine (0.6 mmol), and carbon tetrachloride (0.6 mmol) at room temperature for 12 h. The solution was then concentrated to dryness and purified by Analogix silica gel chromatography (dichloromethane-methanol) to give an off-white solid (80%).

MS (ESP): 748.3 (MH⁺) for C₃₄H₄₄F₃N₉O₅S.

### Intermediate 213

### methyl 5-bromo-2-(3-propylureido)isonicotinate

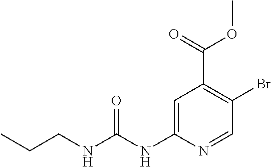

Methyl 2-amino-5-bromoisonicotinate (100 g, 433 mmol) was dissolved in chloroform (600 mL) and placed into a 1 L sealed tube. Propyl isocyanate (122.5 mL, 1.29 mol) was then added. The reaction was heated at 55° C. for 72 h at which time the reaction was determined to be complete. The mixture was then cooled to room temperature, concentrated under reduced pressure, and the solid was dissolved in 2:1 ethyl acetate: tetrahydrofuran (3 L). This solution was washed with water (2×, 200 mL), and the water was back extracted with ethyl acetate (300 mL). The organic layers were then dried with sodium sulfate, filtered, and concentrated yielding 129 g (95%) of methyl 5-bromo-2-(3-propylureido)isonicotinate as a dark yellow solid.

MS (ESP): 316.1 (MH⁺) for C₁₁H₁₄BrN₃O₃

¹H NMR (300 MHz, CDCl₃): δ 0.88 (t, 3H), 1.45 (m, 2H), 3.11 (m, 2H), 3.90 (s, 3H), 7.21 (bt, 1H), 8.02 (s, 1H), 8.46 (s, 1H), 9.40 (s, 1H)

### Intermediate 214

### 5-bromo-2-(3-propylureido)isonicotinamide

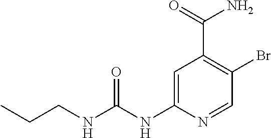

A solution of methyl 5-bromo-2-(3-propylureido)isonicotinate (Intermediate 213, 128 g, 405 mmol) and 7N ammonia in methanol (1 L) was allowed to stir at room temperature for 3 d, then the solids were allowed to settle. The precipitated was vacuum filtered, rinsed with methanol (2×, 500 mL), and dried on the high vacuum pump overnight, yielding 123 g (quant) of 5-bromo-2-(3-propylureido)isonicotinamide as a white solid.

MS (ESP): 301.1 (MH⁺) for C₁₀H₁₃BrN₄OS

¹H NMR (300 MHz, DMSO-d₆): δ 0.88 (t, 3H), 1.46 (m, 2H), 3.18 (q, 2H), 7.41 (bs, 1H), 7.58 (s, 1H), 7.78 (bs, 1H), 8.08 (bs, 1H), 8.33 (s, 1H), 9.31 (s, 1H)

### Intermediate 215

### 5-bromo-2-(3-propylureido)pyridine-4-carbothioamide

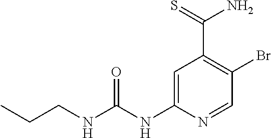

A suspended mixture of 5-bromo-2-(3-propylureido)isonicotinamide (Intermediate 214, 123 g, 407 mmol), Lawesson's Reagent (131.6 g, 326 mmol), and tetrahydrofuran (1.55 L) was stirred at 70° C. for 18 h. Stirring was stopped and a bright yellow precipitate was allowed to settle. The precipitate was vacuum filtered and washed with methyl tert-butyl ether (2×, 500 L). This solid was then dried in the vacuum oven at 50° C. for 12 hours to give 50 g of product solid. The mother liquor was concentrated and the residue was suspended in toluene (300 mL). The solid thus obtained was filtered and combined with the previous solid. The combined totaled 110 g (85%) of 5-bromo-2-(3-propylureido)pyridine-4-carbothioamide as an off white solid

MS (ESP): 317.2 (MH⁺) for C₁₀H₁₃BrN₄OS

¹H NMR (300 MHz, CDCl₃): δ 0.88 (t, 3H), 1.42 (m, 2H), 3.13 (m, 2H), 7.38 (s, 1H), 7.50 (s, 1H), 8.28 (s, 1H), 9.25 (s, 1H), 9.80 (s, 1H), 10.28 (s, 1H)

### Intermediate 216

### 1-(5-bromo-4-(4-hydroxy-4-(trifluoromethyl)-4,5-dihydrothiazol-2-yl)pyridin-2-yl)-3-propylurea

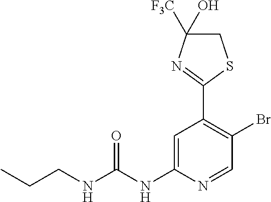

A suspension of 5-bromo-2-(3-propylureido)pyridine-4-carbothioamide (Intermediate 215, 100 g, 315 mmol), 3-bromo-1,1,1-trifluoroacetone (64 mL, 630 mmol) in acetonitrile (1.5 L) was heated at 80° C. for 20 hours. The solution was then cooled down and was concentrated under reduced pressure to give an orange oil that was carried on without further purification.

MS (ESP): 426.9 (MH⁺) for C₁₃H₁₄BrF₃N₄O₂S

¹H NMR (300 MHz, DMSO-d₆): δ 0.88 (t, 3H), 1.48 (m, 2H), 3.11 (m, 2H), 3.62 (d, 1H), 3.92 (d, 1H), 7.30 (bs, 1H), 7.98 (s, 1H), 8.46 (s, 1H), 9.42 (s, 1H).

### Intermediate 217

### 1-(5-bromo-4-(4-(trifluoromethyl)thiazol-2-yl)pyridin-2-yl)-3-propylurea

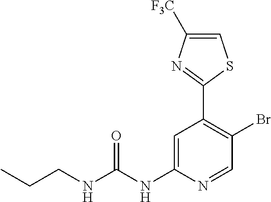

A solution of 1-(5-bromo-4-(4-hydroxy-4-(trifluoromethyl)-4,5-dihydrothiazol-2-yl)pyridin-2-yl)-3-propylurea (Intermediate 216, 315 mmol) and triethylamine (217 mL, 1.57 mol) in tetrahydrofuran (1.3 L) was prepared and stirred at room temperature. Methane sulfonyl chloride (61 mL, 787 mmol) was added dropwise over the course of 1 h. This mixture was stirred at 26° C. for 4 h. Stirring was then stopped and the solids were filtered, washed with tetrahydrofuran (3×, 200 mL), and discarded. The combined tetrahydrofuran layers were concentrated to a viscous, yellow semi-solid which was then triturated with methanol (1 L). The solid was filtered and washed with methanol (2×, 300 mL), then dried in the vacuum oven at 50° C. for 12 h to give 99.4 g (76%) of 1-(5-bromo-4-(4-(trifluoromethyl)thiazol-2-yl)pyridin-2-yl)-3-propylurea as an off-white solid.

MS (ESP): 409.1 (MH⁺) for C₁₃H₁₂BrF₃N₄OS

¹H NMR (300 MHz, DMSO-d₆): δ 0.89 (t, 3H), 1.47 (m, 2H), 3.16 (m, 2H), 7.25 (s, 1H), 8.41 (s, 1H), 8.57 (s, 1H), 8.82 (s, 1H), 9.39 (s, 1H).

### Intermediate 218

### 6-(3-propylureido)-4-(4-(trifluoromethyl)thiazol-2-yl)pyridin-3-ylboronic acid

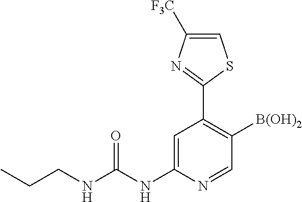

A suspension of 1-(5-bromo-4-(4-(trifluoromethyl)thiazol-2-yl)pyridin-2-yl)-3-propylurea (Intermediate 217, 50 g, 123 mmol) in tetrahydrofuran (1.25 L) was prepared and stirred at −50° C. 2.0 M isopropyl magnesium chloride in tetrahydrofuran (183 mL, 368 mmol) was added dropwise over 45 min so that the temperature never rose above −35° C. The reaction mixture was stirred for a further hour at −40° C. then was cooled to −78° C. A solution of 2.5 M n-butyl lithium in hexanes (295 mL, 735 mmol) was then added dropwise to the reaction solution over the course of 1 h so that the temperature never rose above −65° C. This mixture was then allowed to react at −78° C. for 1.5 h. Boron methoxide (164 mL, 1.47 mol) was added in 1 portion and the cold bath was removed. The reaction was allowed to warm to room temperature and stir for 1 h. 3N Hydrochloric acid (500 mL) was then added slowly to minimize foaming and the reaction was stirred at room temperature for 30 min so that all of the solids dissolved. The reaction was concentrated to remove the tetrahydrofuran and water (1 L) was added. The solution was basified to pH 10 with 24% sodium hydroxide and the total volume was increased to 2 L with water. The aqueous solution was extracted with methyl tert-butyl ether (3×, 650 mL). The organic layers were combined and extracted with 5% sodium hydroxide (100 mL). The aqueous phases were combined and acidified to pH 5.5 with 6N hydrochloric acid causing a suspension to form. This suspension was extracted with 2:1 ethyl acetate: THF (5×, 400 mL) ensuring all solid dissolved in the organic phase. The organic phases were combined and back washed with water (1 L). The organics were concentrated and triturated with methyl tert-butyl ether (1 L). The solid obtained was dried in a vacuum oven at 50° C. for 18 h. This gave 25 g (55%) of 6-(3-propylureido)-4-(4-(trifluoromethyl)thiazol-2-yl)pyridin-3-ylboronic acid as an off-white solid.

MS (ESP): 375.0 (MH⁺) for C₁₃H₁₄BF₃N₄O₃S

¹H NMR (300 MHz, DMSO-d₆): δ 0.90 (t, 3H), 1.45-1.52 (m, 2H), 3.07-3.16 (m, 2H), 7.81 (bt, 1H), 7.91 (s, 1H), 8.20 (br, 2H), 8.31 (d, 1H), 8.65 (m, 1H), 9.32 (s, 1H)

### Intermediate 219

### methyl 2-fluoro-6′-(3-propylureido)-4′-(4-(trifluoromethyl)thiazol-2-yl)-3,3′-bipyridine-5-carboxylate

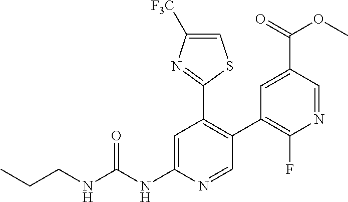

To a slurry of 6-(3-propylureido)-4-(4-trifluoromethylthiazol-2-yl)pyridin-3-ylboronic acid (Intermediate 218, 4.2 g, 11.1 mmol), methyl 5-bromo-6-fluoronicotinate (2.0 g, 8.5 mmol) and trans dichlorobis(triphenylphosphine)palladium (II) (597 mg, 0.85 mmol) in 1,4-dioxane (72 mL) was added a solution of potassium carbonate (2.4 g, 17.0 mmol) in water (27 mL) and the mixture was stirred at 70° C. for 1 h. The reaction was cooled to room temperature, and ethyl acetate (100 mL) and water (10 mL) were added to help separate the layers. The water was removed, and the organic phase was washed with water (10 mL). The organic layer was then concentrated and the resulting residue was triturated with a mixture of ethanol (20 mL) and methyl tert-butyl ether (50 mL). The solid was dried in a vacuum oven at 50° C. for 3 h to give 1.7 g (42% yield) of methyl 2-fluoro-6′-(3-propylureido)-4′-(4-(trifluoromethyl)thiazol-2-yl)-3,3′-bipyridine-5-carboxylate as an off white solid.

MS (ESP): 484.2 (MH⁺) for C₂₀H₁₇F₄N₅O₃S

¹H NMR (300 MHz, DMSO-d₆): δ 0.91 (t, 3H), 1.49 (m, 2H), 3.16 (m, 2H), 3.93 (s, 3H), 7.58 (bt, 1H), 8.23 (s, 1H), 8.39 (s, 1H), 8.48 (dd, 1H), 8.60 (s, 1H), 8.81 (d, 1H), 9.56 (bs, 1H).

### Intermediate 220

### 2-(2-(diisopropylamino)ethoxy)-6′-(3-propylureido)-4′-(4-(trifluoromethyl)thiazol-2-yl)-3,3′-bipyridine-5-carboxylic acid

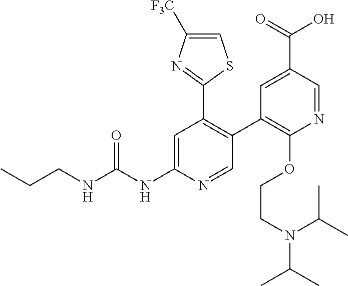

A suspension of methyl 6′-(3-propylureido)-2-fluoro-4′-(4-(trifluoromethyl)thiazol-2-yl)-3,3′-bipyridine-5-carboxylate (Intermediate 219, 0.3 g, 6 mmol) in diisopropylaminoethanol (2-3 mL, ˜50 equiv.) in small vial was stirred for 2 min at room temperature. Sodium hydride (0.150 g, 60 mmol) was added over 5 minutes and the reaction was stirred for a further 5 h at room temperature. The reaction mixture was cooled with an ice bath and slowly quenched with HCl (0.1N) solution until pH 7. The aqueous layer was extracted twice with ether to remove excess alcohol. The aqueous layer was concentrated in vacuo to almost dryness, then loaded on Analogix C18-column for reverse phase purification (water-methanol) to remove excess of salt.

MS (ESP): 594.22 (M+H⁺) for C₂₇H₃₃F₃N₆O₄S.

### Intermediate 221

### 1-(2′-(2-(diisopropylamino)ethoxy)-5′-(5-oxo-4,5-dihydro-1,3,4-oxadiazol-2-yl)-4-(4-(trifluoromethyl)thiazol-2-yl)-3,3′-bipyridin-6-yl)-3-propylurea

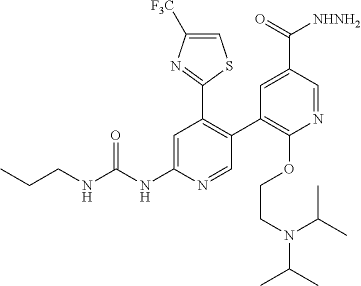

A suspension of 2-(2-(diisopropylamino)ethoxy)-6′-(3-propylureido)-4′-(4-(trifluoromethyl)thiazol-2-yl)-3,3′-bipyridine-5-carboxylic acid (Intermediate 220, 0.3 mmol) in thionyl chloride (2 mL) was heated at 50° C. for 1 h. The solution was then cooled and concentrated to dryness. The crude suspended in tetrahydrofuran (2 mL) was added slowly to a solution of hydrazine/tetrahydrofuran (1/2 vol., 3 mL) and stirred at room temperature for 12 h. The crude reaction mixture was concentrated under reduced pressure to dryness and purified by reverse phase on Analogix C18-column (water-methanol) to give the hydrazide as off-white solid.

MS (ESP): 609.2 (MH⁺) for C₂₇H₃₅F₃N₈O₃S

### Intermediate 222

### 3-Bromo-5-(1H-pyrazol-5-yl)pyridine

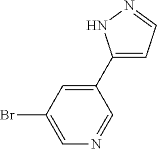

A mixture of 1-(5-Bromopyridin-3-yl)-3-(dimethylamino)prop-2-en-1-one (Intermediate 223, 300 mg, 1.18 mmol) and hydrazine (0.111 mL, 3.53 mmol) in ethanol (3 mL) was heated to reflux for 1.5 h. The solvent was removed and the crude light yellow solid (245 mg), which was used without further purification.

MS (ESP): 226 (M+2) for C₈H₆BrN₃

¹H-NMR (DMSO-d₆) δ: 6.94 (d, 1H); 7.85 (brs, 1H); 8.41 (s, 1H); 8.62 (d, 1H); 9.04 (d, 1H); 13.20 (brs, 1H)

### Intermediate 223

### 1-(5-Bromopyridin-3-yl)-3-(dimethylamino)prop-2-en-1-one

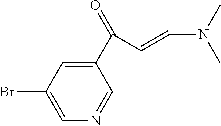

1-(5-Bromopyridin-3-yl)ethanone (1.3 g, 6.50 mmol) and 1,1-dimethoxy-N,N-dimethylmethanamine (5 mL, 6.50 mmol) were taken in a round bottomed flask and heated at 120° C. for 1 h. The reaction mixture was cooled to room temperature and then partitioned between water and ethyl acetate. The layers were separated and the organic layer was washed with water two times, then dried over magnesium sulfate and concentrated under reduced pressure to give a bright orange colored solid (1.4 g) as the product.

MS (ESP): 257 (M+2) for C₁₀H₁₁BrN₂O

### Intermediates 224-233

The following compounds have been synthesized as described for Example 21 from the starting materials indicated in the table below.

| Int | Compound | Data | SM |
|---|---|---|---|
| 224 | 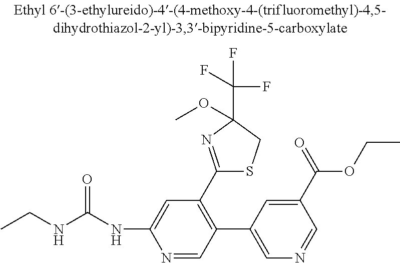 | MS (ESP): 498 (M + 1) for C₂₁H₂₂F₃N₅O₄S | Intermediate 244 and ethyl 5-(4,4,5,5- tetramethyl-1,3,2- dioxaborolan-2- yl)nicotinate |
| 225 | 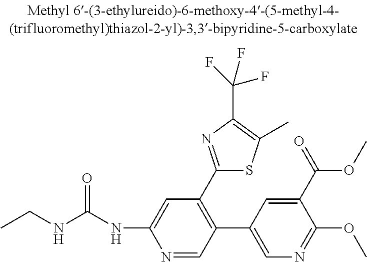 | MS (ESP): 496 (M + 1) for C₂₁H₂₀F₃N₅O₄S | Intermediate 245 and methyl 5-bromo-2- methoxynicotinate. |
| 226 | 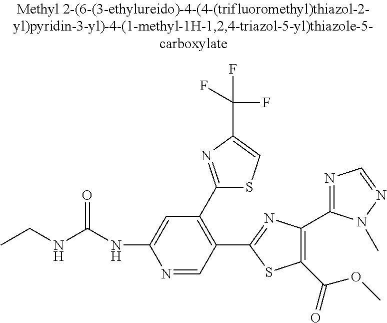 | MS (ESP): 539 (M + 1) for C₂₀H₁₇F₃N₈O₃S₂ | Intermediate 12 and Intermediate 44 |
| 227 | 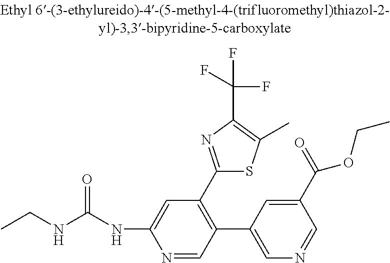 | MS (ESP): 480 (M + 1) for C₂₁H₂₀F₃N₅O₃S ¹H-NMR (DMSO-d₆) δ: 1.11 (t, 3H); 1.32 (t, 3H); 2.53 (s, 3H); 3.12-3.24 (m, 2H); 4.35 (q, 2H); 7.58 (brs, 1H); 8.15 (s, 1H); 8.25 (d, 1H); 8.34 (s, 1H); 8.74 (d, 1H); 9.10 (d, 1H); 9.50 (s, 1H) | ethyl 5-(4,4,5,5- tetramethyl-1,3,2- dioxaboralan-2- yl)nicotinate and Intermediate 247 |
| 228 | 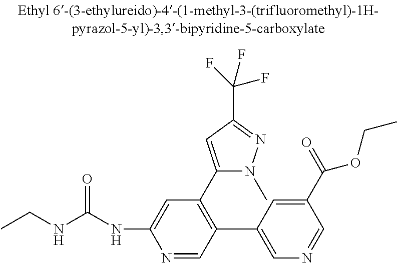 | MS (ESP): 463 (M + 1) for C₂₁H₂₁F₃N₆O₃ | Intermediate 472 and 1-methyl-3- (trifluoromethyl)-1H- pyrazol-5-ylboronic acid |
| 229 | 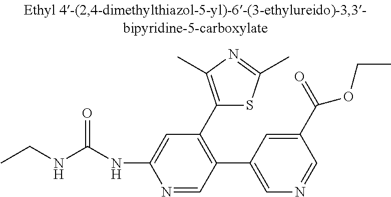 | MS (ESP): 426 (M + 1) for C₂₁H₂₃N₅O₃S | Intermediate 472 and 2,4-dimethylthiazol-5- ylboronic acid |
| 230 | 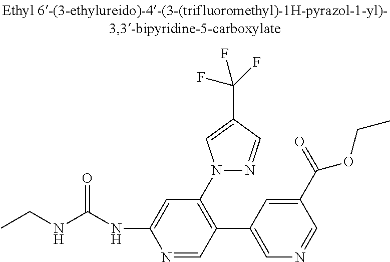 | MS (ESP): 449 (M + 1) for C₂₀H₁₉F₃N₆O₃ | ethyl 5-(4,4,5,5- tetramethyl-1,3,2- dioxaborolan-2- yl)nicotinate and Intermediate 250 |
| 231 | 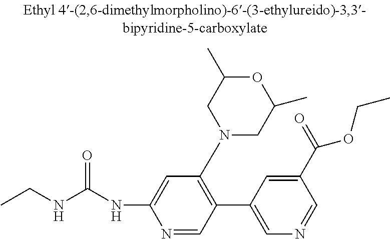 | MS (ESP): 428 (M + 1) for C₂₂H₂₉N₅O₄ | ethyl 5-(4,4,5,5- tetramethyl-1,3,2- dioxaborolan-2- yl)nicotinate and Intermediate 251 |
| 232 | 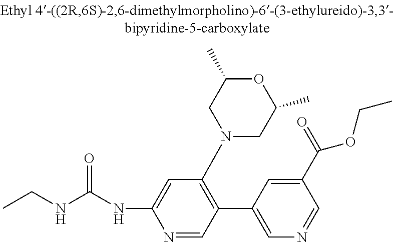 | MS (ESP): 428 (M + 1) for C₂₂H₂₉N₅O₄ | ethyl 5-(4,4,5,5- tetramethyl-1,3,2- dioxaborolan-2- yl)nicotinate and Intermediate 252 |
| 233 | 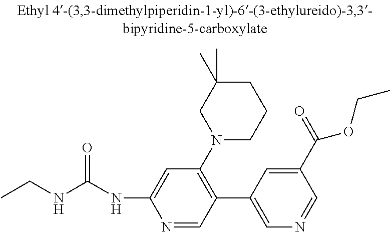 | MS (ESP): 426 (M + 1) for C₂₃H₃₁N₅O₃ | ethyl 5-(4,4,5,5- tetramethyl-1,3,2- dioxaborolan-2- yl)nicotinate and Intermediate 249 |

### Intermediates 234-243

The following compounds have been synthesized as described for Intermediate 9 from the starting materials indicated in the table below.

| Int | Compound | Data | SM |
|---|---|---|---|
| 234 | 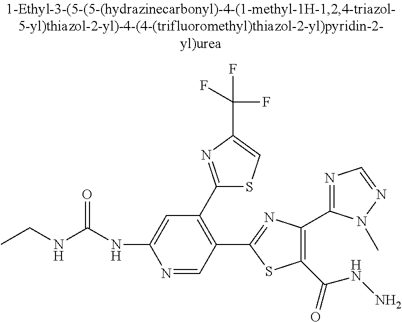 | MS (ESP): 539 (M + 1) for C₁₉H₁₇F₃N₁₀O₂S₂ | Intermediate 226 |
| 235 | 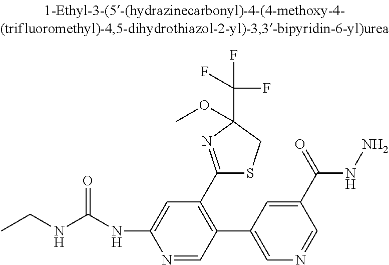 | MS (ESP): 484 (M + 1) for C₁₉H₂₀F₃N₇O₃S | Intermediate 224 |
| 236 | 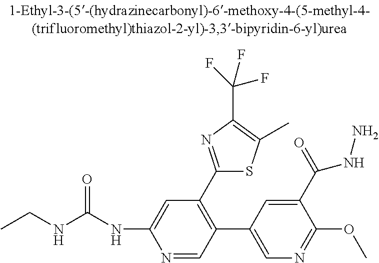 | MS (ESP): 496 (M + 1) for C₂₀H₂₀F₃N₇O₃S. | Intermediate 225 |
| 237 | 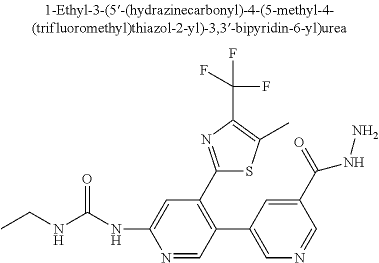 | MS (ESP): 466 (M + 1) for C₁₉H₁₈F₃N₇O₂S | Intermediate 227 |
| 238 | 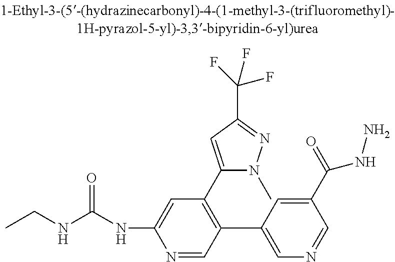 | MS (ESP): 449 (M + 1) for C₁₉H₁₉F₃N₈O₃ | Intermediate 228 |
| 239 | 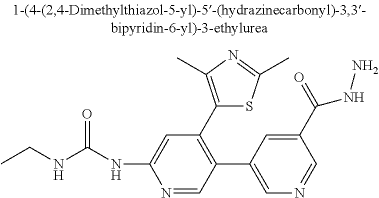 | MS (ESP): 412 (M + 1) for C₁₉H₂₁N₇O₂S | Intermediate 229 |
| 240 | 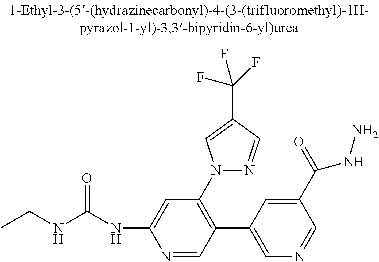 | MS (ESP): 435 (M + 1) for C₁₈H₁₇F₃N₈O₂ | Intermediate 230 |
| 241 | 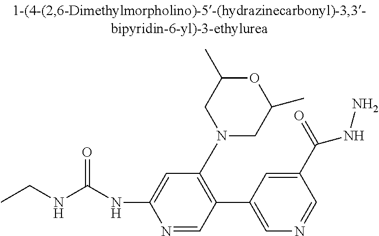 | MS (ESP): 414 (M + 1) for C₂₀H₂₇N₇O₃ | Intermediate 231 |
| 242 | 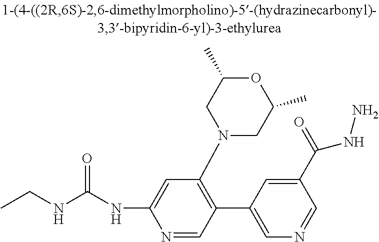 | MS (ESP): 414 (M + 1) for C₂₀H₂₇N₇O₃ | Intermediate 232 |
| 243 | 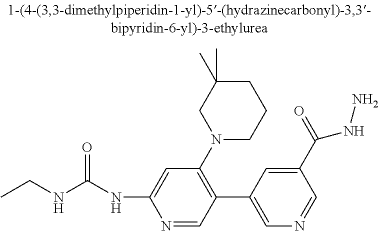 | MS (ESP): 412 (M + 1) for C₂₁H₂₉N₇O₂ | Intermediate 233 |

### Intermediate 244

### 1-(5-Bromo-4-(4-methoxy-4-(trifluoromethyl)-4,5-dihydrothiazol-2-yl)pyridin-2-yl)-3-ethylurea

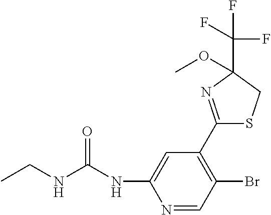

To a suspension of 5-bromo-2-(3-ethylureido)pyridine-4-carbothioamide (Intermediate 5, 25 g, 82.46 mmol) in acetonitrile (150 mL), 3-bromo-1,1,1-trifluoropropan-2-one (12.84 mL, 123.69 mmol) was added and the mixture was heated to reflux for 5 h. The reaction mixture was cooled to room temperature, filtered, and the filtrate was concentrated under reduced pressure. The resulting residue was slurried in ethylaceate, filtered, and washed with methanol. The solid obtained was taken to the next step.

MS (ESP): 429 (M+2) for C₁₃H₁₄BrF₃N₄O₂S

¹H-NMR (DMSO-d₆) δ: 1.07 (t, 3H); 3.08-3.24 (m, 2H); 3.40 (s, 3H); 3.93 (s, 1H); 3.96 (s, 1H); 7.13 (t, 1H); 7.99 (s, 1H); 8.51 (s, 1H); 9.43 (s, 1H).

### Intermediate 245

### 1-Ethyl-3-(4-(5-methyl-4-(trifluoromethyl)thiazol-2-yl)-5-(4,4,5,5-tetramethyl-1,3,2-dioxaborolan-2-yl)pyridin-2-yl)urea

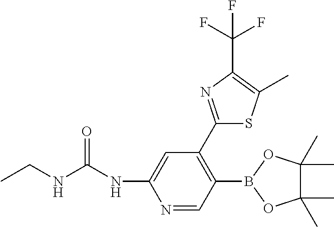

The title compound was synthesized by a method analogous to the synthesis of the Intermediate 12 starting with 1-(5-bromo-4-(5-methyl-4-(trifluoromethyl)thiazol-2-yl)pyridin-2-yl)-3-ethylurea (Intermediate 244) and 4,4,4′,4′,5,5,5′,5′-octamethyl-2,2′-bi(1,3,2-dioxaborolane).

MS (ESP): 457 (M+1) for C₁₉H₂₄BF₃N₄O₃S

### Intermediate 246

### 5-(5-Bromopyridin-3-yl)-3-methyl-1,3,4-oxadiazol-2(3H)-one

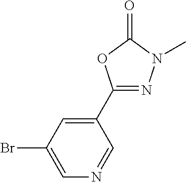

To a 1 M solution of potassium tert-butoxide (413 μl, 0.41 mmol) in THF, 5-(5-bromopyridin-3-yl)-1,3,4-oxadiazol-2(3H)-one (Intermediate 485 100 mg, 0.41 mmol) was added. To this mixture 2 mL of THF was added and the mixture was stirred for 30 min at room temperature. Then methyl iodide (51.7 μl, 0.83 mmol) was added and DMF (2 mL) was added as a co-solvent in order to dissolve the starting material, and the resulting suspension was stirred for an additional 30 min. Then water was added and the precipitated product was isolated by filtration. The precipitate was slurried with acetonitrile, filtered and dried to give a nice white solid (75 mg). MS (ESP): 258 (M+2) for C₈H₆BrN₃O₂

¹H-NMR (DMSO-d₆) δ: 3.44 (s, 3H); 8.37 (t, 1H); 8.91 (d, 1H); 8.95 (d, 1H).

### Intermediate 247

### 1-(5-Bromo-4-(5-methyl-4-(trifluoromethyl)thiazol-2-yl)pyridin-2-yl)-3-ethylurea

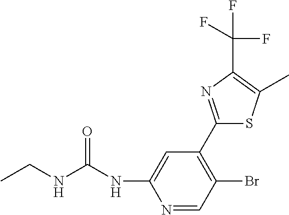

The title compound was synthesized by a method analogous to the synthesis of the Intermediate 3 starting with 1-(5-bromo-4-(4-hydroxy-5-methyl-4-(trifluoromethyl)-4,5-dihydrothiazol-2-yl)pyridin-2-yl)-3-ethylurea (Intermediate 248).

MS (ESP): 411 (M+2) for C₁₃H₁₂BrF₃N₄OS

¹H-NMR (DMSO-d₆) δ: 1.08 (t, 3H); 2.69 (s, 3H); 3.10-3.24 (m, 2H); 7.26 (t, 1H); 8.39 (s, 1H); 8.54 (s, 1H); 9.39 (s, 1H).

### Intermediate 248

### 1-(5-Bromo-4-(4-hydroxy-5-methyl-4-(trifluoromethyl)-4,5-dihydrothiazol-2-yl)pyridin-2-yl)-3-ethylurea

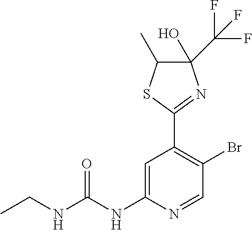

The title compound was synthesized by a method analogous to the synthesis of the intermediate 4 starting with 5-bromo-2-(3-ethylureido)pyridine-4-carbothioamide (Intermediate 5) and 3-bromo-1,1,1-trifluorobutan-2-one.

MS (ESP): 429 (M+2) for C₁₃H₁₄BrF₃N₄O₂S.

### Intermediate 249

### 1-(5-Bromo-4-(3,3-dimethylpiperidin-1-yl)pyridin-2-yl)-3-ethylurea

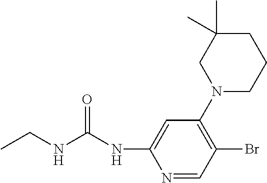

To a solution of 1-(4-(3,3-dimethylpiperidin-1-yl)pyridin-2-yl)-3-ethylurea (Intermediate 253, 200 mg, 0.72 mmol), in DMF (3 mL), N-bromo-succinamide (NBS, 129 mg, 0.72 mmol) was added. The resulting solution was heated at 80° C. for 1 hr. The reaction was then partitioned between water and ethyl acetate. The layers separated and the organic layer was washed with water and brine, then dried over magnesium sulfate, concentrated and the crude was purified by normal phase (ethyl acetate/hex) chromatography. The fractions containing the product were combined and concentrated to give an off-white solid (160 mg).

MS (ESP): 357(M+2) for C₁₅H₂₃BrN₄O.

### Intermediates 250-252

The following compounds have been synthesized as described for Intermediate 249 from the starting materials indicated in the table below.

| Int | Compound | Data | SM |
|---|---|---|---|
| 250 | 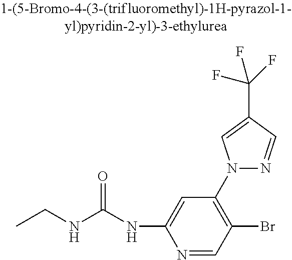 | MS (ESP): 380 (M + 2) for C₁₂H₁₁BrF₃N₅O ¹H-NMR (DMSO-d₆) δ: 1.07 (t, 3H); 3.07-3.28 (m, 2H); 7.11 (d, 1H); 7.16 (brs, 1H); 7.96 (s, 1H); 8.53 (s, 1H); 8.59 (s, 1H); 9.50 (s, 1H) | Intermediate 254 |
| 251 | 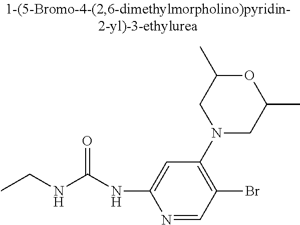 | MS (ESP): 359 (M + 2) for C₁₄H₂₁BrN₄O₂ ¹H-NMR (DMSO-d₆) δ: 1.07 (t, 3H); 1.13 (d, 6H); 2.35 (dd, 2H); 3.08-3.20 (m, 2H); 3.41 (d, 2H); 3.56-3.98 (m, 2H); 7.25 (s, 1H); 7.57 (brs, 1H); 8.14 (s, 1H); 9.03 (s, 1H) | Intermediate 255 |
| 252 | 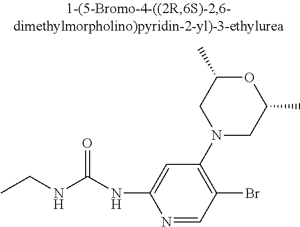 | MS (ESP): 359 (M + 2) for C₁₄H₂₁BrN₄O₂ | Intermediate 256 |

### Intermediate 253

### 1-(4-(3,3-Dimethylpiperidin-1-yl)pyridin-2-yl)-3-ethylurea

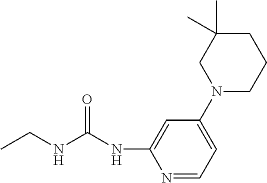

1-(4-Bromopyridin-2-yl)-3-ethylurea (Intermediate 14, 500 mg, 2.05 mmol), 3,3-dimethylpiperidine (301 mg, 2.66 mmol), copper(I) iodide (39.0 mg, 0.20 mmol) and pyrrolidine-2-carboxylic acid (47.2 mg, 0.41 mmol) and potassium carbonate (566 mg, 4.10 mmol) were taken in a round bottomed flask and degassed with argon. DMSO (8 mL) was added, and the mixture was degassed with argon again, then heated at 105° C. for 4 h. The reaction was partitioned between water and ethyl acetate. The layers were separated and the aqueous was extracted with ethyl acetate. The combined extract was washed with water and brine, dried over magnesium sulfate and concentrated. The crude was purified by normal phase (Hex/ethyl acetate) chromatography to give an off-white solid (200 mg).

MS (ESP): 277 (M+1) for C₁₅H₂₄N₄O

### Intermediates 254-256

The following compounds have been synthesized as described for Intermediate 253 from the starting materials indicated in the table below.

| Int | Compound | Data | SM |
|---|---|---|---|
| 254 | 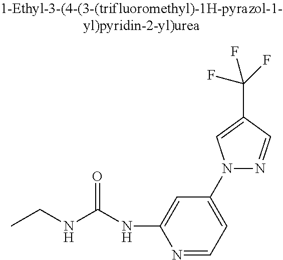 | MS (ESP): 300 (M + 1) for C₁₂H₁₂F₃N₅O ¹H-NMR (DMSO-d₆) δ: 1.10 (t, 3H); 3.07-3.28 (m, 2H); 7.13 (d, 1H); 7.49 (dd, 1H); 7.72 (brs, 1H); 8.07 (d, 1H); 8.31 (d, 1H); 8.85 (d, 1H); 9.35 (s, 1H) | Intermediate 14 and 3- (trifluoromethyl)-1H- pyrazole |
| 255 | 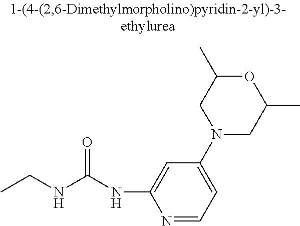 | MS (ESP): 279 (M + 1) for C₁₄H₂₂N₄O₂ | Intermediate 14 and 2,6- dimethylmorpholine |
| 256 | 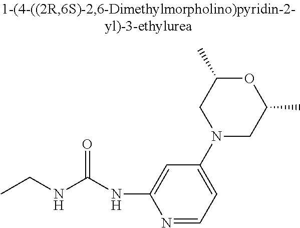 | MS (ESP): 279 (M + 1) for C₁₄H₂₂N₄O₂ | Intermediate 14 and 2R,6S)-2,6- Dimethylmorpholine |

### Intermediate 257

### (S)-tert-Butyl 1-(5-(6′-(3-ethylureido)-4′-(3-(trifluoromethyl)-1H-pyrazol-1-yl)-3,3′-bipyridin-5-yl)-1,3,4-oxadiazol-2-yl)-2-methylpropylcarbamate

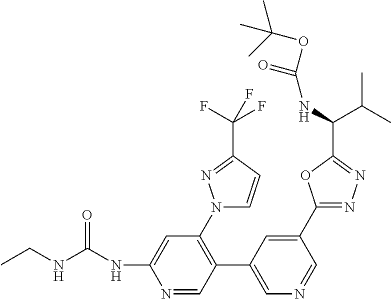

To a solution of 1-ethyl-3-(5′-(hydrazinecarbonyl)-4-(3-(trifluoromethyl)-1H-pyrazol-1-yl)-3,3′-bipyridin-6-yl)urea (Intermediate 238, 80 mg, 0.18 mmol) in DMF (3 mL), DIPEA (0.032 mL, 0.18 mmol) was added followed by (S)-2-(tert-butoxycarbonylamino)-3-methylbutanoic acid (40.0 mg, 0.18 mmol) and HATU (180 mg, 0.47 mmol) and the solution was stirred for 30 min at RT. The reaction was partitioned between water and ethyl acetate. The layers separated and the aqueous layer was back extracted with ethyl acetate twice. Then the combined extracts were washed with water and brine, dried over magnesium sulfate and concentrated to give a clear oil. The oil was taken up in acetonitrile (5 mL) and DBU (0.042 mL, 0.28 mmol) was added followed by triphenylphosphine (97 mg, 0.37 mmol) and carbon tetrachloride (0.036 mL, 0.37 mmol). The resulting solution was stirred at room temperature overnight. The reaction was concentrated and the crude was partitioned between water and ethyl acetate. The layers separated and the aqueous was back extracted twice. The combined extracts were washed with water and dried over magnesium sulfate, concentrated and purified by normal phase (hex/ethyl acetate) chromatography to give a white solid (95 mg) which was slurried in acetonitrile, and filtered and dried (65 mg white solid).

MS (ESP): 616 (M+1) for C₂₈H₃₂F₃N₉O₄

### Intermediate 258

### 1-(5′-(2-(Cyclopropanecarbonyl)hydrazinecarbonyl)-4-(4-(trifluoromethyl)thiazol-2-yl)-3,3′-bipyridin-6-yl)-3-ethylurea

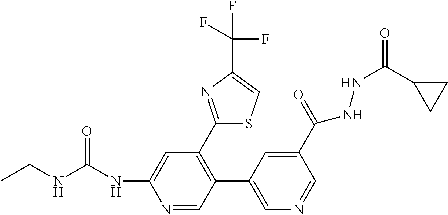

To a suspension of 1-ethyl-3-(5′-(hydrazinecarbonyl)-4-(4-(trifluoromethyl)thiazol-2-yl)-3,3′-bipyridin-6-yl)urea (Intermediate 9, 70 mg, 0.16 mmol) in acetonitrile (3 mL), potassium carbonate (25.7 mg, 0.19 mmol) was added, followed by a slow addition of cyclopropanecarbonyl chloride (0.014 mL, 0.16 mmol) and the resulting mixture was stirred at room temperature for 30 minutes. The precipitated product was filtered and the residue was washed with acetonitrile and water to give 72 mg of the desired product as a tan colored solid.

MS (ESP): 520 (M+1) for C₂₂H₂₀F₃N₇O₃S. Intermediates 259-260

The following Intermediates were prepared by the general procedure described below from the starting materials indicated in the Table.

### General Procedure

A suspension of ester (0.3 g, 6 mmol, 1 eq) in the corresponding alcohol (2-3 mL, ˜50 equiv.) in a small vial was stirred for 2 min at room temperature. Sodium hydride (0.150 g, 60 mmol) was added over 5 min and the reaction was stirred for a further 5 h at room temperature. The reaction was cooled with an ice bath and slowly quenched with HCl (0.1N) solution until pH 7. The aqueous layer was extracted twice with ether to remove excess alcohol. The aqueous layer was concentrated in vacuo to almost dryness than loaded on Analogix C18-column for reverse phase purification (water-methanol) to remove excess salt.

| Int | Compound | Data | SM |
|---|---|---|---|
| 259 | 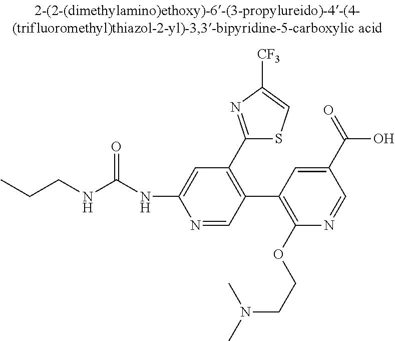 | MS (ESP): 539.1 (MH⁺) for C₂₃H₂₅F₃N₆O₄S | Intermediate 219 and 2-(2- (diisopropylamino) ethanol |
| 260 | 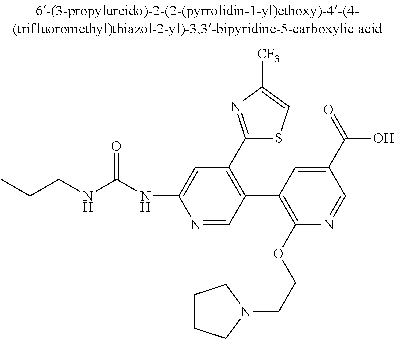 | MS (ESP): 565.1 (MH⁺) for C₂₅H₂₇F₃N₆O₄S | Intermediate 219 and 2-(pyrrolidin-1-yl) ethanol |

### Intermediate 261

### tetrahydro-2H-pyran-4-yl 6′-(3-propylureido)-2-(tetrahydro-2H-pyran-4-yloxy)-4′-(4-(trifluoromethyl)thiazol-2-yl)-3,3′-bipyridine-5-carboxylate

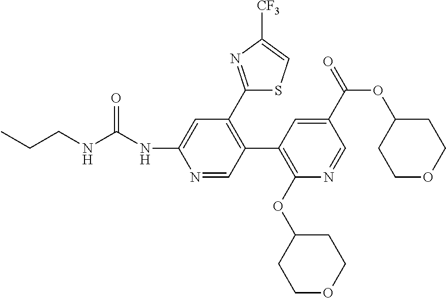

To a slurry of 6-(3-propylureido)-4-(4-trifluoromethylthiazol-2-yl)pyridin-3-ylboronic acid (Intermediate 218, 0.63 g, 1.7 mmol), tetrahydro-2H-pyran-4-yl 5-bromo-6-(tetrahydro-2H-pyran-4-yloxy)nicotinate (Intermediate 281, 0.5 g, 1.3 mmol) and trans dichlorobis(triphenylphosphine)palladium (II) (597 mg, 0.85 mmol) in 1,4-dioxane (72 mL) was added a solution of potassium carbonate (2.4 g, 17.0 mmol) in water (27 mL). The reaction was stirred at 70° C. for 1 h. The reaction was cooled to room temperature, and ethyl acetate (100 mL) and water (10 mL) were added to help separate the layers. The water was removed, and the organic phase was washed with water (10 mL). The reaction was then concentrated and the residue was triturated with a mixture of ethanol (20 mL) and methyl tert-butyl ether (50 mL). The solid was dried in a vacuum oven at 50° C. for 3 hours. This gave tetrahydro-2H-pyran-4-yl 6′-(3-propylureido)-2-(tetrahydro-2H-pyran-4-yloxy)-4′-(4-(trifluoromethyl)thiazol-2-yl)-3,3′-bipyridine-5-carboxylate (˜80% yield) as an off white solid.

MS (ESP): 635.20 (MH⁺) for C₂₉H₃₂F₃N₅O₆S.

### Intermediate 262

### 6′-(3-propylureido)-2-(tetrahydro-2H-pyran-4-yloxy)-4′-(4-(trifluoromethyl)thiazol-2-yl)-3,3′-bipyridine-5-carboxylic acid

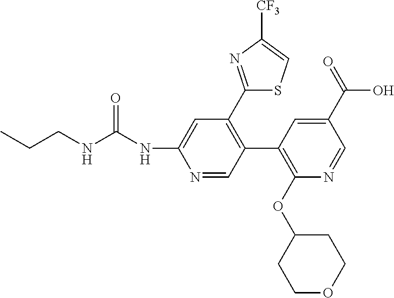

Tetrahydro-2H-pyran-4-yl 6′-(3-propylureido)-2-(tetrahydro-2H-pyran-4-yloxy)-4′-(4-(trifluoromethyl)thiazol-2-yl)-3,3′-bipyridine-5-carboxylate (Intermediate 261, ˜0.3 mmol) was dissolved in tetrahydrofuran-water and treated with lithium hydroxide (10 eq) at room temperature for 24 h. After this period of time, the organic was removed under vacuum. Dilute HCl was added to adjust the pH to 7 and the aqueous layer was extracted with ethyl acetate. The organic layer was dried over sodium sulfate and concentrated to dryness to give the corresponding acid which was used directly for the next step.

MS (ESP): 552.1 (MH⁺) for C₂₄H₂₄F₃N₅O₅S

### Intermediate 263

### Methyl 6′-(3-propylureido)-4′-(4-(trifluoromethyl)thiazol-2-yl)-3,3′-bipyridine-5-carboxylate

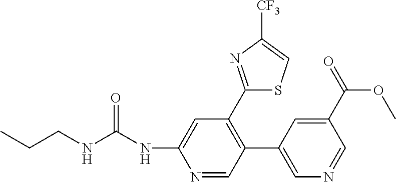

1-(5-bromo-4-(4-(trifluoromethyl)thiazol-2-yl)pyridin-2-yl)-3-propylurea (Intermediate 218, 200 mg, 0.51 mmol), methyl 5-(4,4,5,5-tetramethyl-1,3,2-dioxaborolan-2-yl)nicotinate (173 mg, 0.65 mmol), and trans dichlorobis(triphenylphosphine)palladium (II) (36 mg, 0.05 mmol) were dissolved in 1,4-dioxane (8 mL). Sodium bicarbonate (170 mg, 2 mmol) was dissolved in water (3 mL) and added to the above mixture. The reaction was heated at 65° C. for 60 min. Ethyl acetate (10 mL) was then added to the reaction and the layers were separated. The solvent was removed in vacuo and the residue was triturated with ethanol (5 mL). The solid was dried in a vacuum oven at 60° C. for 1 hour to give 145 mg (64% yield) of methyl 6′-(3-propylureido)-4′-(4-(trifluoromethyl)thiazol-2-yl)-3,3′-bipyridine-5-carboxylate as an off white powder.

MS (ESP): 466.2 (M+H⁺) for C₂₀H₁₈F₃N₅O₃S

¹H NMR (300 MHz, DMSO-d₆): δ 0.88 (t, 3H), 1.49 (m, 2H), 3.17 (m, 2H), 3.87 (s, 3H), 7.59 (bt, 1H), 8.20 (s, 1H), 8.21 (s, 1H), 8.37 (s, 1H), 8.37 (s, 1H), 8.75 (d, 1H), 9.07 (d, 1H), 9.54 (bs, 1H).

### Intermediate 264

### Diethyl 24643-propylureido)-4-(4-(trifluoromethyl)thiazol-2-yl)pyridin-3-yl)thiazole-4,5-dicarboxylate

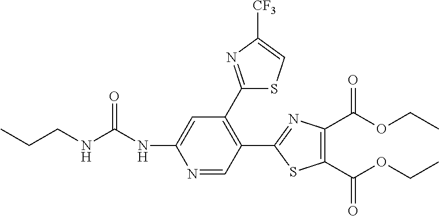

6-(3-Propylureido)-4-(4-(trifluoromethyl)thiazol-2-yl)pyridin-3-ylboronic acid (Intermediate 218, 200 mg, 0.54 mmol), diethyl 2-chlorothiazole-4,5-dicarboxylate (110 mg, 0.41 mmol), and trans dichlorobis(triphenylphosphine)palladium (II) (30 mg, 0.041 mmol) were dissolved in 1,4-dioxane (8 mL). Sodium bicarbonate (170 mg, 2 mmol) was dissolved in water (3 mL) and added to the above mixture. The reaction was heated at 80° C. in a microwave for 60 min. Ethyl acetate (10 mL) was then added to the reaction and the layers were separated. The solvent was removed in vacuo and the residue was chromatographed on an Analogix 4 g column using 1-100% ethyl acetate in heptane. This gave 73 mg (31% yield) of diethyl 2-(6-(3-propylureido)-4-(4-(trifluoromethyl)thiazol-2-yl)pyridin-3-yl)thiazole-4,5-dicarboxylate as an off white powder.

MS (ESP): 558.2 (M-FH') for C₂₂H₂₂F₃N₅O₅S₂

### Intermediate 265

### Methyl 6-(3-propylureido)-4-(4-(trifluoromethyl)thiazol-2-yl)-3,4′-bipyridine-2′-carboxylate

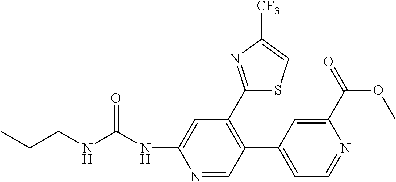

6-(3-Propylureido)-4-(4-(trifluoromethyl)thiazol-2-yl)pyridin-3-ylboronic acid (Intermediate 218, 311 mg, 0.86 mmol), methyl 4-chloropicolinate (135 mg, 0.78 mmol), and trans dichlorobis(triphenylphosphine)palladium (II) (32 mg, 0.04 mmol) were dissolved in 1,4-dioxane (4 mL). Sodium bicarbonate (131 mg, 1.5 mmol) was dissolved in water (1 mL) and added to the above mixture. The reaction was heated at 80° C. for 60 min in the microwave. Ethyl acetate (10 mL) was then added to the reaction and the layers were separated. The solvent was removed in vacuo and the residue was chromatographed on an Analogix 4 g column using 0-100% ethyl acetate in heptane. The solid was dried in a vacuum oven at 60° C. for 1 hour to give 102 mg (26% yield) of methyl 6-(3-propylureido)-4-(4-(trifluoromethyl)thiazol-2-yl)-3,4′-bipyridine-2′-carboxylate as an off white powder.

MS (ESP): 466.2 (M+H⁺) for C₂₀H₁₈F₃N₅O₃S

### Intermediate 266

### 1-Ethyl-3-(5′-(2-(3-hydroxyazetidine-1-carbonyl)hydrazinecarbonyl)-4-(4-(trifluoromethyl)thiazol-2-yl)-3,3′-bipyridin-6-yl)urea

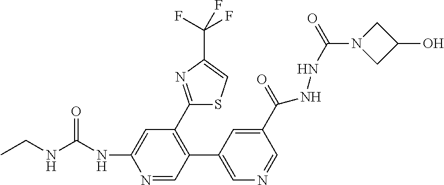

To a suspension of 1-ethyl-3-(5′-(5-oxo-4,5-dihydro-1,3,4-oxadiazol-2-yl)-4-(4-(trifluoromethyl)thiazol-2-yl)-3,3′-bipyridin-6-yl)urea (Example 6, 235 mg, 0.49 mmol) in ethanol (3 mL) was added azetidin-3-ol (162 mg, 1.48 mmol) followed by TEA (0.206 mL, 1.48 mmol) and heated to 100° C. for 2 h in a microwave. The reaction mixture was concentrated and used without further purification.

### Intermediate 267

### (R)-1-Ethyl-3-(5′-(2-(3-hydroxypyrrolidine-1-carbonyl)hydrazinecarbonyl)-4-(4-(trifluoromethyl)thiazol-2-yl)-3,3′-bipyridin-6-yl)urea

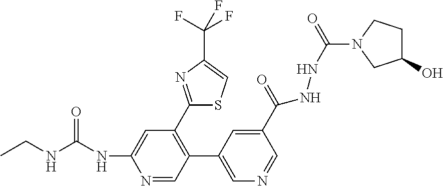

The title compound was synthesized by method analogous to Intermediate 266 starting with Example 6 and (R)-pyrrolidin-3-ol.

### Intermediate 268

### (S)-1-ethyl-3-(5′-(2-(3-hydroxypyrrolidine-1-carbonyl)hydrazinecarbonyl)-4-(4-(trifluoromethyl)thiazol-2-yl)-3,3′-bipyridin-6-yl)urea

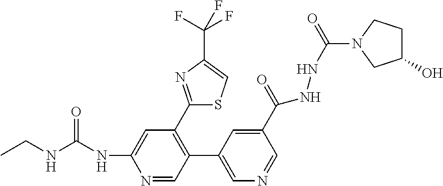

The title compound was synthesized by method analogous to Intermediate 266 starting with Example 6 and (S)-pyrrolidin-3-ol.

### Intermediate 269

### 1-Ethyl-3-(5′-(2-(4-hydroxypiperidine-1-carbonyl)hydrazinecarbonyl)-4-(4-(trifluoromethyl)thiazol-2-yl)-3,3′-bipyridin-6-yl)urea

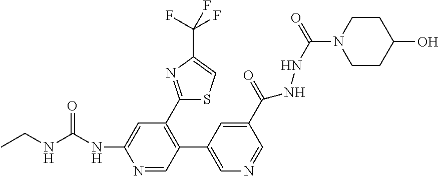

The title compound was synthesized by method analogous to Intermediate 266 starting with Example 6 and piperidin-4-ol.

### Intermediate 270

### 1-Ethyl-3-(5′-(2-(3-hydroxypiperidine-1-carbonyl)hydrazinecarbonyl)-4-(4-(trifluoromethyl)thiazol-2-yl)-3,3′-bipyridin-6-yl)urea

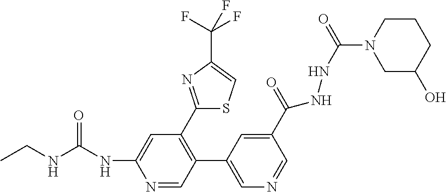

The title compound was synthesized by method analogous to Intermediate 266 starting with Example 6 and piperidin-3-ol.

### Intermediate 271

### (S)-tert-butyl 1-(2-(6′-(3-ethylureido)-4′-(4-(trifluoromethyl)thiazol-2-yl)-3,3′-bipyridine-5-carbonyl)hydrazinyl)-3-methyl-1-oxobutan-2-ylcarbamate

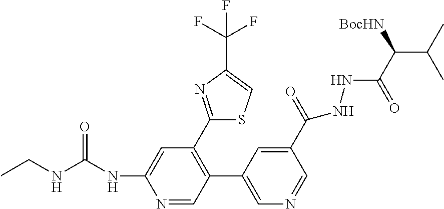

To a solution of 1-ethyl-3-(5′-(hydrazinecarbonyl)-4-(4-(trifluoromethyl)thiazol-2-yl)-3,3′-bipyridin-6-yl)urea (Intermediate 9, 340 mg, 0.75 mmol) in DMF (5 mL) was added (S)-2-(tert-butoxycarbonylamino)-3-methylbutanoic acid (245 mg, 1.13 mmol), HATU (573 mg, 1.51 mmol) and DIEA (0.329 mL, 1.88 mmol). After stirring overnight, the reaction mixture was diluted with water and extracted with EtOAc (2×). The combined organic layers were washed with brine, dried (Na₂SO₄) and concentrated to give light yellow color solid.

### Intermediate 172

### (R)-tert-butyl 1-(2-(6′-(3-ethylureido)-4′-(4-(trifluoromethyl)thiazol-2-yl)-3,3′-bipyridine-5-carbonyl)hydrazinyl)-3-methyl-1-oxobutan-2-ylcarbamate

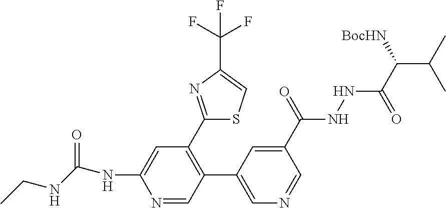

The title compound was synthesized by method analogous to Intermediate 271 starting with Intermediate 9 and (R)-2-(tert-butoxycarbonylamino)-3-methylbutanoic acid.

### Intermediates 273-280

The following Intermediates were prepared according to the procedure described for Intermediate 28 from the starting materials indicated in the Table.

| Int | Compound | Data | SM |
|---|---|---|---|
| 273 | 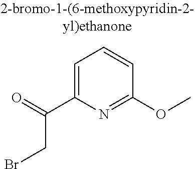 | LC/MS (ES⁺)(M + H)⁺: 230 232 for C₈H₈BrNO₂. ¹H NMR (300 MHz, CDCl3): 4.01 (s, 3H), 4.81 (s, 2H), 7.0 (d, 1H), 7.73 (m, 2H). | 1-(6-methoxypyridin- 2-yl)ethanone |
| 274 | 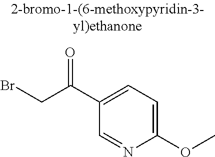 | LC/MS (ES⁺)(M + H)⁺: 230 232 for C₈H₈BrNO₂. ¹H NMR (300 MHz, d₆-DMSO): 3.95 (s, 3H), 4.90 (s, 2H), 6.96 (d, 1H), 8.21 (m, 1H), 8.88 (s, 1H). | 1-(6-methoxypyridin- 3-yl)ethanone |
| 275 | 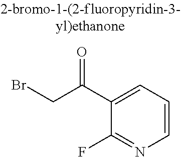 | LC/MS (ES⁺)[(M + H)⁺]: 216 218 for C₇H₅BrFNO. ¹H NMR (300 MHz, d₆-DMSO): 4.86 (s, 2H), 6.41 (m, 1H), 7.82 (m, 1H), 8.17 (m, 1H). | 1-(2-fluoropyridin- 3-yl)ethanone |
| 276 | 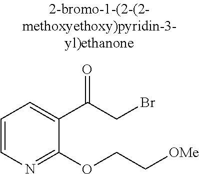 | LC/MS (ES⁺)[(M + H)⁺]: 274, 276 for C₁₀H₁₂BrNO₃. ¹H NMR (300 MHz, d₆-DMSO): 3.33 (s, 3H), 3.74 (m, 2H), 4.53 (m, 2H), 4.84 (s, 2H), 7.18 (m, 1H), 8.14 (m, 1H), 8.41 (m, 1H). | 1-(2-(2- methoxyethoxy- pyridin-3-yl) ethanone |
| 277 | 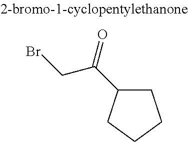 | ¹H NMR (300 MHz, CDCl3): 1.69 (m, 8H), 3.18 (m, 1H), 3.99 (s, 2H). | 1-cyclopentyl-ethanone |
| 278 | 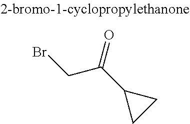 | ¹H NMR (300 MHz, CDCl3): 1.02 (m, 2H), 1.14 (m, 2H), 2.22 (m, 1H), 4.02 (s, 2H). | 1-cyclopentyl-ethanone |
| 279 | 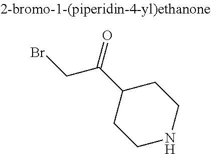 | ¹H NMR (300 MHz, CDCl3): 1.69 (m, 8H), 3.18 (m, 1H), 3.99 (s, 2H). | benzyl 4- acetylpiperidine-1- carboxylate |
| 280 | 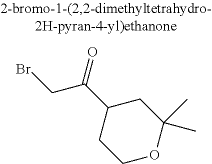 | ¹H NMR (300 MHz, CDCl3): 1.26 (s, 3H), 1.27 (s, 3H), 1.56 (m, 2H), 1.73 (m, 2H), 3.15 (m, 1H), 3.75 (m, 2H), 3.96 (s, 2H). | 1-(2,2- dimethyltetrahydro- 2H-pyran-4-yl) ethanone |

### Intermediate 281

### Tetrahydro-2H-pyran-4-yl 5-bromo-6-(tetrahydro-2H-pyran-4-yloxy)nicotinate

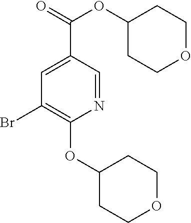

In a dried 250 mL glass round bottom flask, sodium hydride (0.878 g, 21.96 mmol) was suspended in 20 mL of anhydrous DMF. Tetrahydro-2H-pyran-4-ol (1.838 mL, 19.32 mmol) was added to the suspension dropwise. Upon reacting, the suspension became homogenous and a clear yellow solution was obtained. Methyl 5-bromo-6-chloronicotinate (2.2 g, 8.78 mmol) was then added in a single portion. The resultant reaction mixture was stirred at room temperature for 1 hour. A brown precipitate began to form. The reaction was monitored by LC/MS and TLC (6:4 EtOAc/hexanes). When the reaction was complete the mixture was diluted with Et₂O, cooled to 0° C. (ice bath) and slowly quenched with water. The aqueous and organic phases were separated and the organic layer was dried over Na₂SO₄, filtered, concentrated by rotary evaporation and purified by flash column chromatography (1:1 EtOAc/hexanes). Isolation gave 441 mg of desired product.

LC/MS (ES⁺): 386, 388 for C₁₆H₂₀BrNO₅.

### Intermediates 282-284

The following Intermediates were prepared according to the procedure described for Intermediate 51 using the starting material indicated in the table.

| Int | Compound | Data | SM |
|---|---|---|---|
| 282 | 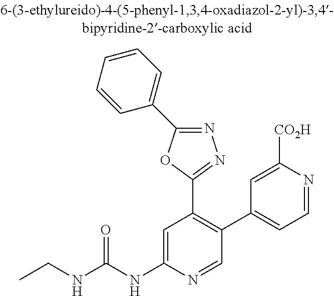 | LC/MS (ES⁺)[(M + H)⁺]: 431 for C₂₂H₁₈N₆O₄. | Intermediate 310 |
| 283 | 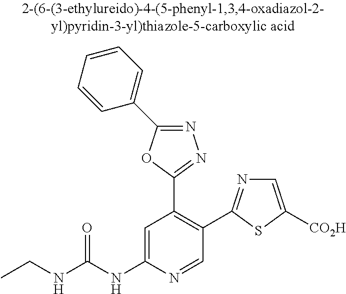 | LC/MS (ES⁺)[(M + H)⁺]: 437 for C₂₀H₁₆N₆O₄S. | Intermediate 312 |
| 284 | 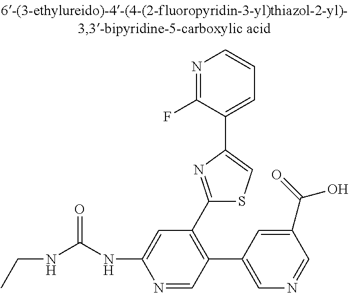 | LC/MS (ES⁺)[(M + H)⁺]: 465 for C₂₂H₁₇FN₆O₃S. ¹H NMR (300 MHz, d₆-DMSO): 1.11 (t, 3H), 3.23 (m, 2H), 7.44 (m, 1H), 7.62 (m, 1H), 8.13 (m, 1H), 8.16 (m, 1H), 8.19 (m, 1H), 8.23 (m, 1H), 8.24 (m, 1H), 8.34 (s, 1H), 8.75 (d, 1H), 9.08 (d, 1H), 9.49 (s, 1H), 13.52 (s, 1H). | Intermediate 320 |

### Intermediate 285

### 1-(4-(2-benzoylhydrazinecarbonyl)-5-bromopyridin-2-yl)-3-ethylurea

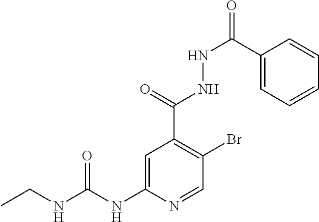

5-bromo-2-(3-ethylureido)isonicotinic acid (Intermediate 51, 6.25 g, 21.69 mmol) was dissolved in a DMF (60 mL) solution containing HATU (12.38 g, 32.54 mmol) and DIEA (7.54 mL, 43.39 mmol). After the mixture was stirred for 15 min, benzohydrazide (3.25 g, 23.86 mmol) was added in a single portion and the reaction mixture was stirred at room temperature for 1 h, the heated to 70° C. for 1 hour. Solids precipitated from solution. The reaction was not complete after 12 hours, so another 2 grams of HATU was added and the mixture was heated until the reaction was complete. The reaction mixture was cooled to room temperature. The solids were filtered and washed with minimal DMF. The solids were then triturated in water, filtered and dried in vacuo. Isolation gave 3 grams of a white fluffy solid. LC/MS (ES⁺): 406, 408 for C₁₆H₁₆BrN₅O₃.

¹H NMR (300 MHz, d₆-DMSO): 1.09 (t, 3H), 3.18 (m, 2H), 7.33 (t, 1H), 7.53 (m, 3H), 7.85 (s, 1H), 7.93 (d, 2H), 8.42 (s, 1H), 9.42 (s, 1H), 10.60 (s, 1H), 10.67 (s, 1H).

### Intermediate 286

The following Intermediate was prepared according to the procedure described for Intermediate 285 using the starting materials indicated in the table.

| Int | Compound | Data | SM |
|---|---|---|---|
| 286 | 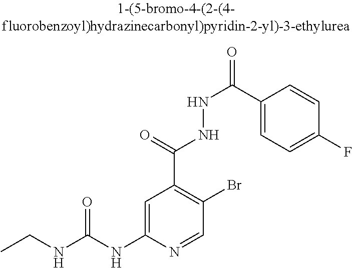 | LC/MS (ES⁺)[(M + H)⁺]: 424, 426 for C₁₆H₁₅BrFN₅O₃. ¹H NMR (300 MHz, d₆-DMSO): 1.08 (t, 3H), 3.17 (m, 2H), 7.38 (m, 3H), 7.87 (s, 1H), 8.01 (m, 2H), 8.41 (s, 1H), 9.46 (s, 1H), 10.62 (s, 1H), 10.72 (s, 1H). | Intermediate 51 and 4-fluorobenzo- hydrazide |

### Intermediate 287

### 1-(5-bromo-4-(5-phenyl-1,3,4-oxadiazol-2-yl)pyridin-2-yl)-3-ethylurea

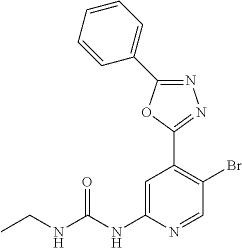

1-(4-(2-benzoylhydrazinecarbonyl)-5-bromopyridin-2-yl)-3-ethylurea (Intermediate 285, 4.73 g, 11.64 mmol) was suspended in a methylene chloride (20 mL) solution containing triphenyl phosphine (3.36 g, 12.81 mmol), carbon tetrabromide (4.25 g, 12.81 mmol) and triethylamine (1.790 mL, 12.81 mmol) (pre-mixed and stirred for 10 minutes). The mixture was stirred at room temperature and monitored by LC/MS. The reaction was not complete after 12 hours, so a second CH₂Cl₂ (20 mL) solution containing triphenyl phosphine (3.36 g, 12.81 mmol), carbon tetrabromide (4.25 g, 12.81 mmol) and triethylamine (1.790 mL, 12.81 mmol) was prepared and added to the reaction mixture. This procedure was repeated a third time. Once the reaction was deemed complete, the precipitate was filtered off, and washed with CH₂Cl₂. The filtration yielded 970 mg of the product. The mother liquor was purified by flash column chromatography (95:5 CH₂Cl₂/MeOH). Isolation gave another 1.2 gram of product. Total isolated weight was 2.1 g.

LC/MS (ES⁺): 388, 340 for C₁₆H₁₄BrN₅O₂.

¹H NMR (300 MHz, d₆-DMSO): 1.09 (t, 3H), 3.19 (m, 2H), 7.21 (t, 1H), 7.66 (m, 2H), 7.69 (s, 1H), 8.08 (m, 2H), 8.45 (s, 1H), 8.61 (s, 1H), 9.49 (s, 1H).

### Intermediate 288

The following Intermediate was prepared according to the procedure described for Intermediate 287 using the starting materials indicated in the table.

| Int | Compound | Data | SM |
|---|---|---|---|
| 288 | 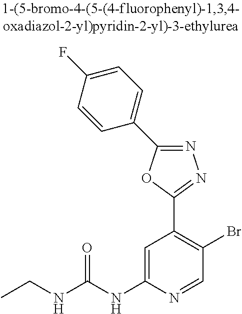 | LC/MS (ES⁺): 406, 408 for C₁₆H₁₃BrFN₅O₂. ¹H NMR (300 MHz, d₆-DMSO): 1.09 (t, 3H), 3.17 (m, 2H), 7.19 (t, 1H), 7.53 (m, 2H), 8.15 (m, 2H), 8.46 (s, 1H), 8.62 (s, 1H), 9.49 (s, 1H). | Intermediate 286 |

### Intermediate 289

### Ethyl 2-(4-carbamothioyl-6-(3-ethylureido)pyridin-3-yl)-4-(pyrimidin-2-yl)thiazole-5-carboxylate

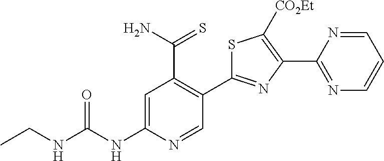

Ethyl 2-(4-carbamoyl-6-(3-ethylureido)pyridin-3-yl)-4-(pyrimidin-2-yl)thiazole-5-carboxylate (Intermediate 319, 132 mg, 0.30 mmol) was suspended in THF. Lawesson's reagent (145 mg, 0.36 mmol) was added in a single portion. The suspension was heated to reflux for 1 h. The reaction mixture concentrated to dryness.

LC/MS (ES⁺)[(M+H)⁺]: 458 for C₁₉H₁₉N₇O₃S₂.

### Intermediates 290-299

The following Intermediates were prepared according to the procedure described for Intermediate 16 using the starting materials indicated.

| Int | Compound | Data | SM |
|---|---|---|---|
| 290 | 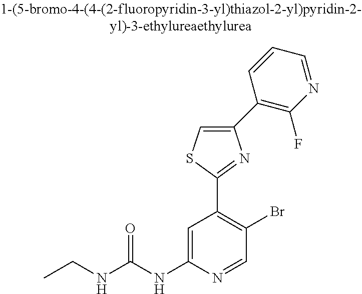 | LC/MS (ES⁺)[(M + H)⁺]: 420, 422 for C₁₆H₁₃BrN₅OS. ¹H NMR (300 MHz, d₆-DMSO): 1.09 (t, 3H), 3.18 (m, 2H), 6.45 (m, 1H), 7.37 (m, 1H), 8.51 (m, 1H), 8.46 (s, 1H), 8.47 (m, 1H), 8.54 (s, 1H), 8.83 (s, 1H), 9.38 (s, 1H). | Intermediate 5 and Intermediate 275 |
| 291 | 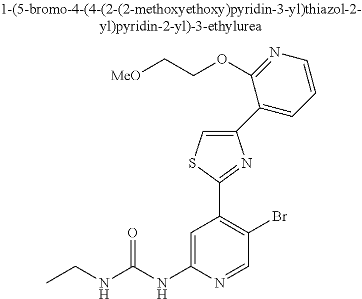 | LC/MS (ES⁺)[(M + H)⁺]: 478, 480 for C₁₉H₂₀BrN₅O₃S. | Intermediate 5 and Intermediate 276 |
| 292 | 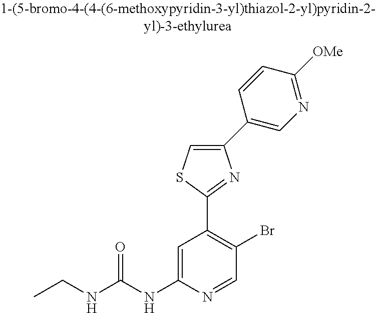 | LC/MS (ES⁺)[(M + H)⁺]: 434, 436 for C₁₇H₁₆BrN₅O₂S. ¹H NMR (300 MHz, d₆-DMSO): 1.09 (t, 3H), 3.18 (m, 2H), 3.92 (s, 3H), 6.98 (d, 1H), 7.34 (m, 1H), 8.32 (m, 1H), 8.45 (s, 1H), 8.53 (s, 1H), 8.54 (s, 1H), 8.87 (d, 1H), 9.38 (s, 1H). | Intermediate 5 and Intermediate 274 |
| 293 | 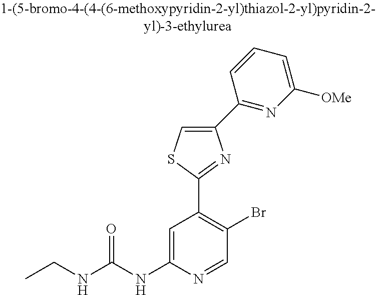 | LC/MS (ES⁺)[(M + H)⁺]: 434, 436 for C₁₇H₁₆BrN₅O₂S. | Intermediate 5 and Intermediate 273 |
| 294 | 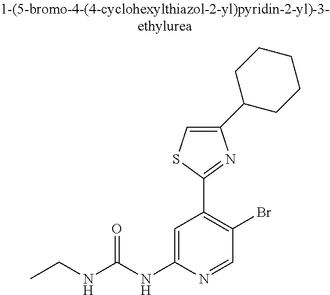 | LC/MS (ES⁺) [(M + H)⁺]: 409, 411 for C₁₇H₂₁BrN₄OS. ¹H NMR (300 MHz, d₆-DMSO): 1.06 (t, 3H), 1.23 (m, 1H), 1.38 (m, 2H), 1.45 (m, 2H), 1.74 (m, 3H), 2.03 (m, 2H), 2.81 (m, 1H), 3.17 (m, 2H), 7.33 (m, 1H), 7.63 (s, 1H), 8.37 (s, 1H), 8.49 (s, 1H), 9.33 (s, 1H). | Intermediate 5 and 2-bromo-1-cyclohexyl-ethanone |
| 295 | 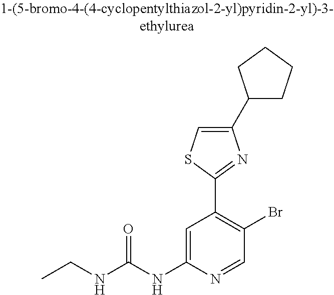 | LC/MS (ES⁺) [(M + H)⁺]: 395, 397 for C₁₆H₁₉BrN₄OS. ¹H NMR (300 MHz, d₆-DMSO): 1.26 (t, 3H), 1.57 (m, 4H), 1.82 (m, 4H), 3.30 (m, 1H), 3.43 (m, 2H), 7.19 (m, 1H), 7.31 (s, 1H), 7.61 (s, 1H), 8.41 (s, 1H), 8.80 (m, 1H). | Intermediate 5 and Intermediate 277 |
| 296 | 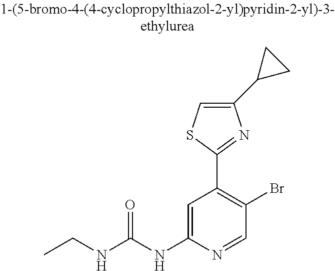 | LC/MS (ES⁺) [(M + H)⁺]: 367, 369 for C₁₄H₁₅BrN₄OS. ¹H NMR (300 MHz, d₆-DMSO): 0.88 (m, 2H), 0.99 (m, 2H), 1.08 (t, 3H), 2.19 (m, 1H), 3.17 (m, 2H), 7.38 (m, 1H), 7.82 (s, 1H), 8.30 (s, 1H), 8.48 (s, 1H), 9.32 (s, 1H). | Intermediate 5 and Intermediate 278 |
| 297 | 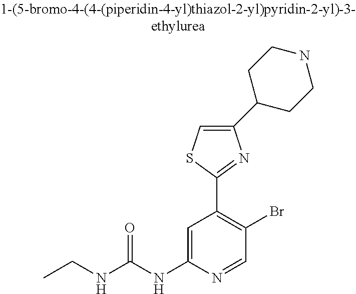 | LC/MS (ES⁺) [(M + H)⁺]: 410, 412 for C₁₆H₂₀BrN₅OS. ¹H NMR (300 MHz, d₆-DMSO): 1.07 (t, 3H), 1.89 (m, 2H), 2.17 (m, 2H), 3.08 (m, 2H), 3.14 (m, 2H), 3.37 (m, 2H), 3.40 (m, 1H), 7.16 (m, 1H), 7.77 (s, 1H), 8.43 (s, 1H), 8.51 (s, 1H), 9.33 (s, 1H). | Intermediate 5 and Intermediate 279 |
| 298 | 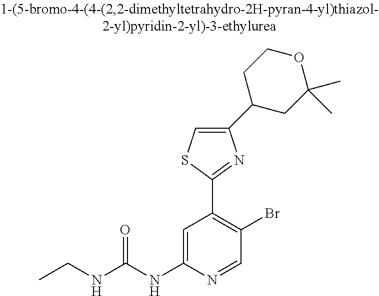 | LC/MS (ES⁺) [(M + H)⁺]: 439 441 for C₁₈H₂₃BrN₄O₂S. ¹H NMR (300 MHz, d₆-DMSO): 1.08 (t, 3H), 1.11 (m, 2H), 1.19 (s, 3H), 1.27 (s, 3H), 1.53 (m, 2H), 1.87 (m, 2H), 3.16 (m, 1H), 3.74 (m, 2H), 7.35 (m, 1H), 7.67 (s, 1H), 8.34 (s, 1H), 8.50 (s, 1H), 9.33 (s, 1H). | Intermediate 5 and Intermediate 280 |
| 299 | 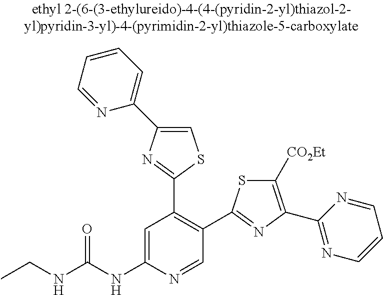 | LC/MS (ES⁺) [(M + H)⁺]: 559 for C₂₆H₂₂N₈O₃S₂. | Intermediate 289 and 2-bromo-1-(pyridin-2-yl)ethanone |

### Intermediate 300

### 1-ethyl-3-(4-(5-phenyl-1,3,4-oxadiazol-2-yl)-5-(4,4,5,5-tetramethyl-1,3,2-dioxaborolan-2-yl)pyridin-2-yl)urea

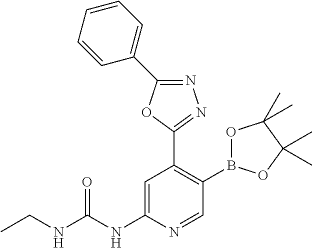

1-(5-Bromo-4-(5-phenyl-1,3,4-oxadiazol-2-yl)pyridin-2-yl)-3-ethylurea (Intermediate 287, 1.17 g, 3.01 mml) and PdCl₂(PPh₃)₂ (0.2 g, 0.3 mmol) were suspended in 1,4 dioxane. The reaction mixture was degassed and purged with nitrogen. The suspension was gently warmed to 70° C. 4,4,4′,4′,5,5,5′,5′-Octamethyl-2,2′-bi(1,3,2-dioxaborolane) (2.3 g, 9.04 mmol) was added in a single portion and the mixture was stirred at 100° C. for 30 min. Triethylamine (0.91 g, 9.04 mmol) was added followed by KOAc (0.88 g, 9.04 mmol). The reaction mixture was then allowed to react for 12 hours, then the reaction was cooled to room temperature, filtered through a pad of Celite, and the mother liquor was concentrated to dryness. The residue was dissolved in ethyl acetate and the solution was washed with water, dried over Na₂SO₄, filtered and concentrated to a solid. The solid was triturated in EtOAc, filtered and dried in vacuo (isolated ˜925 mg's). The mother liquor was concentrated further and then purified by flash column chromatography (0-100% EtOAc/hexanes) to give an additional 60 mg of product. Isolated weight and approximate purity as judged by LC/MS ratios (1:1 ratio of ester and acid): 925 mg (95% pure).

LC/MS (ES⁺)[(M+H)⁺]: 436 for C₂₂H₂₆BN₅O₄ (Boronic ester); 354 for C₁₆H₁₆BN₅O₄ (Boronic acid).

### Intermediate 301-303

The following Intermediates were prepared by the procedure described for Intermediate 300 using the starting materials indicated in the table.

| Int | Compound | Data | SM |
|---|---|---|---|
| 301 | methyl 2-(3-ethylureido)-5- (4,4,5,5-tetramethyl-1,3,2- dioxaborolan-2-yl)isonicotinate 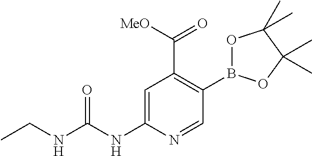 | LC/MS (ES⁺)[(M + H)⁺]: 350 for C₁₆H₂₄BN₃O₅ | Intermediate 321 |
| 302 | 6-(3-ethylureido)-4-(4-(1-methyl-1H- pyrazol-4-yl)thiazol-2- yl)pyridin-3-ylboronic acid 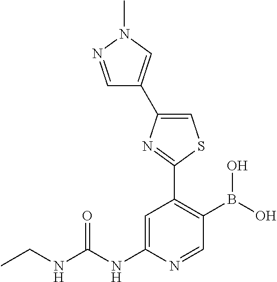 | LC/MS (ES⁺)[(M + H)⁺]: 373 for C₁₅H₁₇BN₆O₃S. ¹H NMR (300 MHz, d₆-DMSO): 1.10 (t, 3H), 3.20 (m, 2H), 3.9 (s, 3H), 7.81 (m, 1H), 7.84 (s, 1H), 7.91 (s, 1H), 7.92 (s, 1H), 8.12 (s, 1H), 8.33 (s, 1H), 8.37 (s, 2H), 9.27 (s, 1H). | Intermediate 29 |
| 303 | 6-(3-ethylureido)-4-(4-(2-(2- methoxyethoxy)pyridin-3- yl)thiazol-2-yl)pyridin-3- ylboronic acid 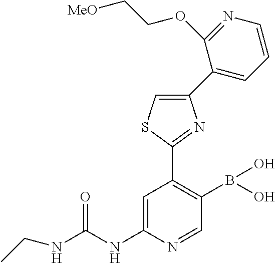 | LC/MS (ES⁺)[(M + H)⁺]: 444 for C₁₉H₂₂BN₅O₅S | Intermediate 291 |

### Intermediate 304

### 4-carbamoyl-6-(3-ethylureido)pyridin-3-ylboronic acid

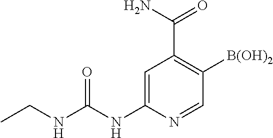

In a sealed microwave vessel, methyl 2-(3-ethylureido)-5-(4,4,5,5-tetramethyl-1,3,2-dioxaborolan-2-yl)isonicotinate (Intermediate 301, 1.2 g, 3.44 mmol) was dissolved in a methanol solution containing ammonia (7N) (10 mL, 70.00 mmol). The solution was heated at 80° C. for 15 minutes, the concentrated to dryness. The product was used without further purification.

LC/MS (ES⁺)[(M+H)⁺]: 369 for C₉H₁₃BN₄O₄.

### Intermediates 305-320

The following Intermediates were prepared according to the procedure described for Intermediate 2 using the starting materials indicated in the table.

| Int | Compound | Data | SM |
|---|---|---|---|
| 305 | methyl 6-(3-ethylureido)-4-(4-(trifluoromethyl)thiazol- 2-yl)-3,4′-bipyridine-2′-carboxylate 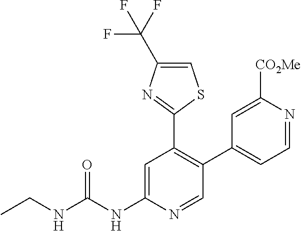 | LC/MS (ES⁺)[(M + H)⁺]: 452 for C₁₉H₁₆F₃N₅O₃S. ¹H NMR (300 MHz, d₆-DMSO): 1.11 (t, 3H), 3.20 (m, 2H), 3.86 (s, 3H), 7.53 (t, 1H), 7.59 (dd, 1H), 7.98 (s, 1H), 8.16 (s, 1H), 8.38 (s, 1H), 8.61 (s, 1H), 8.70 (d, 1H), 9.55 (s, 1H). | Intermediate 12 and methyl 4- bromopicolinate |
| 306 | ethyl 2-(6-(3-ethylureido)-4-(4-(trifluoromethyl)thiazol- 2-yl)pyridin-3-yl)thiazole-4-carboxylate 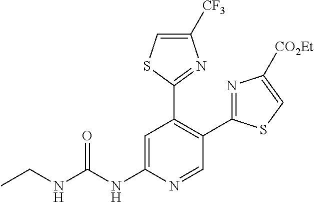 | LC/MS (ES⁺)[(M + H)⁺]: 472 for C₁₈H₁₆F₃N₅O₃S₂. ¹H NMR (300 MHz, d₆-DMSO): 1.11 (t, 3H), 1.29 (t, 3H), 3.22 (m, 2H), 4.28 (q, 2H), 7.49 (t, 1H), 8.14 (s, 1H), 8.65 (s, 2H), 8.69 (s, 1H), 9.67 (s, 1H). | Intermediate 12 and ethyl 2- bromothiazole-4- carboxylate |
| 307 | methyl 6′-(3-ethylureido)-6-methoxy-4′-(4- (trifluoromethyl)thiazol-2-yl)-3,3′-bipyridine-5-carboxylate 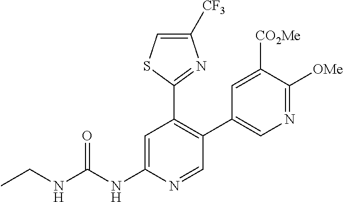 | LC/MS (ES⁺)[(M + H)⁺]: 482 for C₂₀H₁₈F₃N₅O₄S. ¹H NMR (300 MHz, d₆-DMSO): 1.10 (t, 3H), 3.21 (m, 2H), 3.78 (s, 3H), 3.97 (s, 3H), 7.59 (m, 1H), 8.06 (s, 1H), 8.23 (s, 1H), 8.31 (s, 1H), 8.35 (s, 1H), 8.56 (s, 1H), 9.45 (s, 1H). | Intermediate 12 and methyl 5-bromo-2- methoxynicotinate |
| 308 | methyl 5-(6-(3-ethylureido)-4-(4-(trifluoromethyl)thiazol- 2-yl)pyridin-3-yl)pyrazine-2-carboxylate 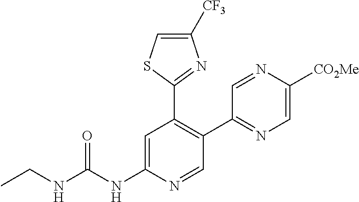 | LC/MS (ES⁺)[(M + H)⁺]: 453 for C₁₈H₁₅F₃N₆O₃S. ¹H NMR (300 MHz, d₆-DMSO): 1.11 (t, 3H), 3.17 (m, 2H), 3.94 (s, 3H), 7.53 (t, 1H), 8.16 (s, 1H), 8.61 (s, 1H), 8.63 (s, 1H), 8.88 (d, 1H), 9.13 (d, 1H), 9.66 (s, 1H). | Intermediate 12 and methyl 5- chloropyrazine-2- carboxylate |
| 309 | 6-(6-(3-ethylureido)-4-(4-(trifluoromethyl)thiazol- 2-yl)pyridin-3-yl)pyridazine-3-carboxylic acid 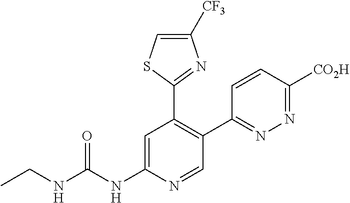 | LC/MS (ES⁺)[(M + H)⁺]: 439 for C₁₇H₁₃F₃N₆O₃S. | Intermediate 12 and 6- chloropyridazine-3- carboxylic acid |
| 310 | methyl 6-(3-ethylureido)-4-(5-phenyl-1,3,4- oxadiazol-2-yl)-3,4′-bipyridine-2′-carboxylate 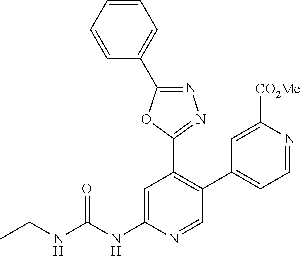 | LC/MS (ES⁺)[(M + H)⁺]: 445 for C₂₃H₂₀N₆O₄. | Intermediate 300 and methyl 4- bromopicolinate |
| 311 | ethyl 6′-(3-ethylureido)-4′-(5-phenyl-1,3,4- oxadiazol-2-yl)-3,3′-bipyridine-5-carboxylate 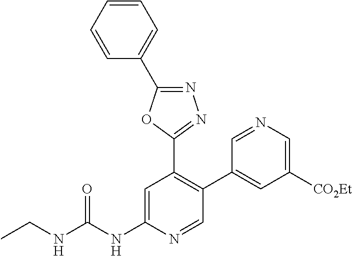 | LC/MS (ES⁺)[(M + H)⁺]: 459 for C₂₄H₂₂N₆O₄. | Intermediate 300 and ethyl 5-bromo- nicotinate |
| 312 | methyl 2-(6-(3-ethylureido)-4-(5-phenyl-1,3,4- oxadiazol-2-yl)pyridin-3-yl)thiazole-5-carboxylate 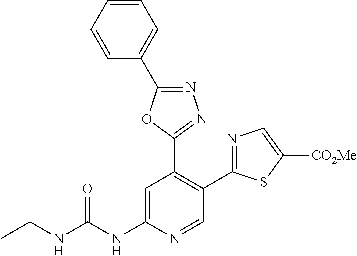 | LC/MS (ES⁺)[(M + H)⁺]: 451 for C₂₁H₁₈N₆O₄S. | Intermediate 300 and methyl 2- bromothiazole-5- carboxylate |
| 313 | ethyl 2-(6-(3-ethylureido)-4-(5-phenyl-1,3,4- oxadiazol-2-yl)pyridin-3-yl)thiazole-4-carboxylate 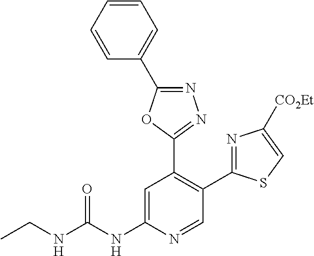 | LC/MS (ES⁺)[(M + H)⁺]: 465 for C₂₂H₂₀N₆O₄S. ¹H NMR (300 MHz, d₆-DMSO): 1.09 (m, 6H), 3.21 (m, 2H), 4.10 (q, 2H), 7.48 (t, 1H), 7.60 (m, 3H), 7.81 (d, 2H), 8.30 (s, 1H), 8.67 (s, 1H), 8.77 (s, 1H), 9.79 (s, 1H). | Intermediate 300 and ethyl 2- bromothiazole-4- carboxylate |
| 314 | methyl 5-(6-(3-ethylureido)-4-(5-phenyl-1,3,4- oxadiazol-2-yl)pyridin-3-yl)pyrazine-2-carboxylate 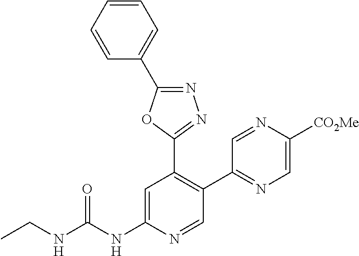 | LC/MS (ES⁺)[(M + H)⁺]: 446 for C₂₂H₁₉N₇O₄. | Intermediate 300 and methyl 5- bromopyrazine-2- carboxylate |
| 315 | methyl 6′-(3-ethylureido)-6-methoxy-4′-(5-phenyl- 1,3,4-oxadiazol-2-yl)-3,3′-bipyridine-5-carboxylate 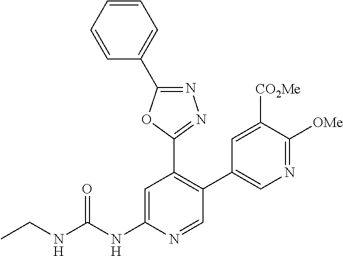 | LC/MS (ES⁺)[(M + H)⁺]: 475 for C₂₄H₂₂N₆O₅. ¹H NMR (300 MHz, d₆-DMSO): 1.12 (t, 3H), 3.22 (m, 2H), 3.78 (s, 3H), 4.00 (s, 3H), 7.52 (t, 1H), 7.68 (m, 3H), 7.77 (d, 2H), 8.22 (s, 1H), 8.38 (s, 1H), 8.45 (m, 2H), 9.53 (s, 1H). | Intermediate 300 and methyl 5-bromo-2- methoxynicotinate |
| 316 | ethyl 6′-(3-ethylureido)-4′-(5-(4-fluorophenyl)-1,3,4- oxadiazol-2-yl)-3,3′-bipyridine-5-carboxylate 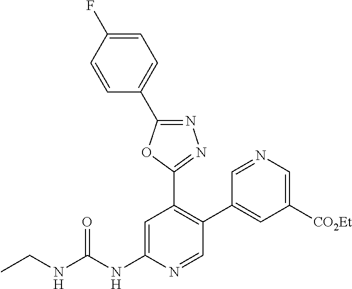 | LC/MS (ES⁺)[(M + H)⁺]: 477 for C₂₄H₂₁FN₆O₄. ¹H NMR (300 MHz, d₆-DMSO): 1.07 (t, 3H), 1.11 (t, 3H), 3.21 (m, 2H), 4.13 (q, 2H), 7.43 (m, 3H), 7.51 (t, 1H), 7.59 (t, 1H), 7.88 (d, 2H), 8.21 (s, 1H), 8.39 (s, 1H), 8.73 (s, 1H), 8.92 (d, 2H), 9.68 (s, 1H). | Intermediate 288 and ethyl 5-(4,4,5,5- tetramethyl-1,3,2- dioxaborolan-2- yl)nicotinate |
| 317 | ethyl 2-(6-(3-ethylureido)-4-(4-phenylthiazol-2- yl)pyridin-3-yl)-4-(pyrimidin-2-yl)thiazole-5-carboxylate 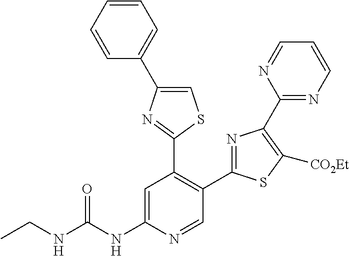 | LC/MS (ES⁺)[(M + H)⁺]: 558 for C₂₇H₂₃N₇O₃S₂. ¹H NMR (300 MHz, d₆-DMSO): 1.07 (t, 3H), 1.11 (t, 3H), 3.21 (m, 2H), 4.13 (q, 2H), 7.43 (m, 3H), 7.51 (t, 1H), 7.59 (t, 1H), 7.88 (d, 2H), 8.21 (s, 1H), 8.39 (s, 1H), 8.73 (s, 1H), 8.92 (d, 2H), 9.68 (s, 1H). | Intermediate 161 and Intermediate 43 |
| 318 | methyl 2-(6-(3-ethylureido)-4-(4-phenylthiazol-2-yl)pyridin-3- yl)-4-(4-methyl-4H-1,2,4-triazol-3-yl)thiazole-5-carboxylate 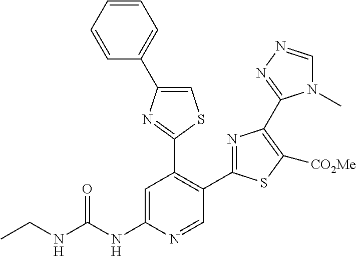 | LC/MS (ES⁺)[(M + H)⁺]: 547 for C₂₅H₂₂N₈O₃S₂. ¹H NMR (300 MHz, d₆-DMSO): 1.11 (t, 3H), 3.21 (m, 2H), 3.60 (s, 3H), 3.75 (s, 3H), 7.40 (m, 3H), 7.51 (t, 1H), 7.84 (d, 2H), 8.06 (s, 1H), 8.17 (s, 1H), 8.36 (s, 1H), 8.76 (s, 2H), 9.71 (s, 1H). | Intermediate 161 and Intermediate 44 |
| 319 | ethyl 2-(4-carbamoyl-6-(3-ethylureido)pyridin- 3-yl)-4-(pyrimidin-2-yl)thiazole-5-carboxylate 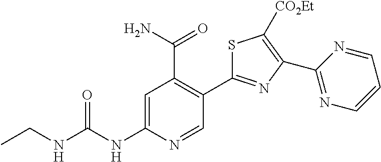 | LC/MS (ES⁺)[(M + H)⁺]: 442 for C₁₉H₁₉N₇O₄S. ¹H NMR (300 MHz, d₆-DMSO): 1.11 (t, 3H), 3.19 (m, 2H), 4.16 (q, 2H), 7.57 (t, 1H), 7.62 (t, 1H), 7.66 (s, 1H), 7.97 (s, 1H), 8.32 (s, 1H), 8.79 (s, 1H), 8.95 (d, 2H), 9.66 (s, 1H). | Intermediate 304 and Intermediate 43 |
| 320 | Ethyl 6′-(3-ethylureido)-4′-(4-(2-fluoropyridin-3- yl)thiazol-2-yl)-3,3′-bipyridine-5-carboxylate 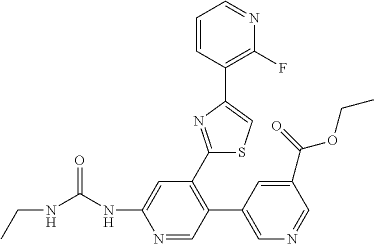 | LC/MS (ES⁺)[(M + H)⁺]: 493 for C₂₄H₂₁FNO₃S. ¹H NMR (300 MHz, d₆-DMSO): 1.11 (t, 3H), 1.27 (t, 3H), 3.22 (m, 2H), 4.32 (m, 2H), 7.26 (m, 1H), 7.43 (s, 1H), 7.61 (s, 1H), 8.11 (m, 1H), 8.20 (m, 1H), 8.22 (m, 1H), 8.25 (m, 1H), 8.36 (s, 1H), 8.79 (d, 1H), 9.10 (d, 1H), 9.50 (s, 1H). | Intermediate 290 and ethyl 5-(4,4,5,5- tetramethyl-1,3,2- dioxaborolan-2- yl)nicotinate |

### Intermediate 321

### Methyl 5-bromo-2-(3-ethylureido)isonicotinate

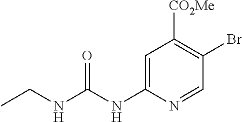

Methyl 2-amino-5-bromoisonicotinate (20 g, 86.56 mmol) was suspended in CHCl₃ (20 mL). Ethyl isocyanate (10.20 mL, 129.84 mmol) was added in a single portion and the reaction was heated in an oil bath to 80° C. for 5 h. The reaction mixture was concentrated to dryness by rotary evaporation. The product crystallized from a mixture of CH₂Cl₂ and hexanes. Isolation gave 16.2 grams of the title compound.

LC/MS (ES⁺): 302, 304 for C₁₀H₁₂BrN₃O₃.

¹H NMR (300 MHz, d₆-DMSO): 1.07 (t, 3H), 3.18 (m, 2H), 3.89 (s, 3H), 7.18 (t, 1H), 8.02 (s, 1H), 8.46 (s, 1H), 9.42 (s, 1H).

### Intermediates 322-335

The following Intermediates were prepared according to the procedure described for Intermediate 2 using the starting materials indicated in the table.

| Int | Compound | Data | SM |
|---|---|---|---|
| 322 | methyl 6′-(3-ethylureido)-6-methoxy-4′-(4-(2-(2- methoxyethoxy)pyridin-3-yl)thiazol-2-yl)-3,3′- bipyridine-5-carboxylate 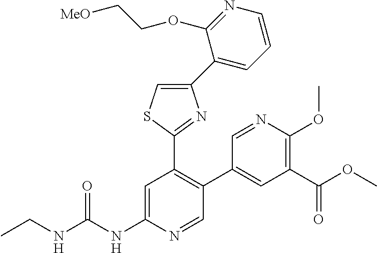 | LC/MS (ES⁺)[(M + H)⁺]: 565 for C₂₇H₂₈N₆O₆S. ¹H NMR (300 MHz, d₆-DMSO): 1.11 (t, 3H), 3.22 (m, 2H), 3.32 (s, 3H), 3.77 (s, 3H), 3.78 (m, 2H), 3.97 (s, 3H), 4.53 (m, 2H), 7.08 (m, 1H), 7.68 (m, 1H), 8.13 (d, 1H), 8.16 (m, 1H), 8.22 (m, 1H), 8.26 (s, 1H), 8.28 (s, 2H), 8.36 (d, 1H), 9.44 (s, 1H) | Intermediate 303 and methyl 5-bromo-2- methoxynicotinate |
| 323 | ethyl 6′-(3-ethylureido)-4′-(4-(2-(2-methoxyethoxy)pyridin- 3-yl)thiazol-2-yl)-3,3′-bipyridine-5-carboxylate 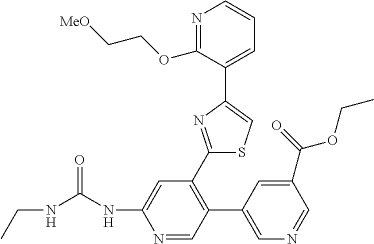 | LC/MS (ES⁺)[(M + H)⁺]: 549 for C₂₇H₂₈N₆O₅S. | Intermediate 291 and ethyl 5-(4,4,5,5- tetramethyl-1,3,2- dioxaborolan-2- yl)nicotinate |
| 324 | tetrahydro-2H-pyran-4-yl 6′-(3-ethylureido)-4′-(4-(2-(2- methoxyethoxy)pyridin-3-yl)thiazol-2-yl)-2-(tetrahydro-2H-pyran-4- yloxy)-3,3′-bipyridine-5-carboxylate 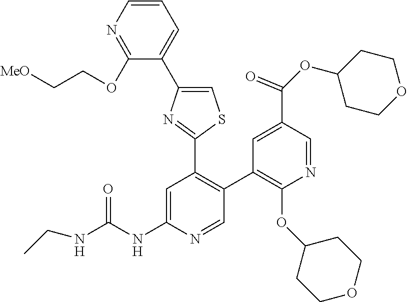 | LC/MS (ES⁺)[(M + H)⁺]: 705 for C₃₅H₄₀N₆O₈S. | Intermediate 303 and Intermediate 281 |
| 325 | Ethyl 6′-(3-ethylureido)-4′-(4-(6-methoxypyridin-2- yl)thiazol-2-yl)-3,3′-bipyridine-5-carboxylate 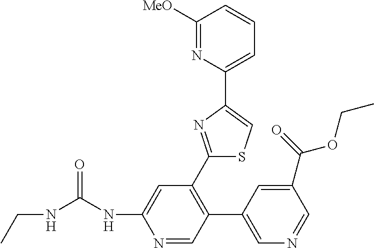 | LC/MS (ES⁺)[(M + H)⁺]: 505 for C₂₅H₂₄N₆O₄S. ¹H NMR (300 MHz, d₆-DMSO): 1.11 (t, 3H), 1.27 (t, 3H), 3.22 (m, 2H), 3.91 (s, 3H), 4.32 (m, 2H), 6.77 (m, 1H), 7.22 (m, 1H), 7.65 (m, 1H), 7.73 (m, 1H), 8.26 (m, 2H), 8.34 (s, 1H), 8.36 (s, 1H), 8.79 (s, 1H), 9.10 (s, 1H), 9.49 (s, 1H). | Intermediate 293 and ethyl 5-(4,4,5,5- tetramethyl-1,3,2- dioxaborolan-2- yl)nicotinate |
| 326 | Ethyl 6′-(3-ethylureido)-4′-(4-(6-methoxypyridin-3-yl)thiazol-2-yl)- 3,3′-bipyridine-5-carboxylate 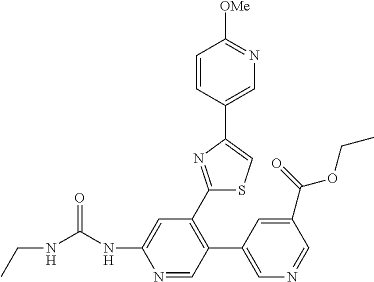 | LC/MS (ES⁺)[(M + H)⁺]: 505 for C₂₅H₂₄N₆O4S. ¹H NMR (300 MHz, d₆-DMSO): 1.11 (t, 3H), 1.28 (t, 3H), 3.22 (m, 2H), 3.88 (s, 3H), 4.33 (m, 2H), 6.86 (m, 1H), 7.62 (m, 1H), 7.99 (m, 1H), 8.20 (m, 1H), 8.25 (s, 1H), 8.27 (t, 1H), 8.33 (s, 1H), 8.50 (m, 1H), 8.78 (d, 1H), 9.10 (d, 1H), 9.48 (s, 1H). | Intermediate 292 and ethyl 5-(4,4,5,5- tetramethyl-1,3,2- dioxaborolan-2- yl)nicotinate |
| 327 | ethyl 4′-(4-cyclohexylthiazol-2-yl)-6′-(3-ethylureido)- 3,3′-bipyridine-5-carboxylate 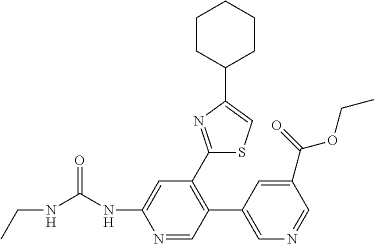 | LC/MS (ES⁺)[(M + H)⁺]: 480 for C₂₅H₂₉N₅O₃S. ¹H NMR (300 MHz, d₆-DMSO): 1.11 (t, 3H), 1.17 (m, 2H), 1.3 (m, 3H), 1.62 (m, 2H), 1.68 (m, 2H), 2.55 (m, 2H), 3.20 (m, 2H), 4.02 (m, 1H), 4.31 (m, 2H), 7.37 (s, 1H), 7.50 (m, 1H), 7.64 (m, 2H), 8.09 (s, 1H), 8.10 (m, 1H), 8.29 (s, 1H), 8.69 (d, 1H), 9.05 (d, 1H), 9.43 (s, 1H). | Intermediate 294 and ethyl 5-(4,4,5,5- tetramethyl-1,3,2- dioxaborolan-2- yl)nicotinate |
| 328 | ethyl 4′-(4-cyclopentylthiazol-2-yl)-6′-(3-ethylureido)- 3,3′-bipyridine-5-carboxylate 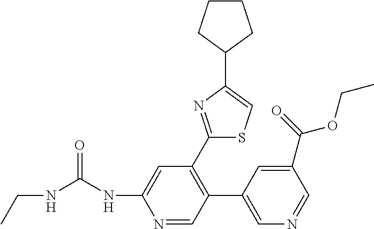 | LC/MS (ES⁺)[(M + H)⁺]: 466 for C₂₄H₂₇N₅O₃S. ¹H NMR (300 MHz, d₆-DMSO): 1.11 (t, 3H), 1.28 (t, 3H), 1.33 (m, 2H), 1.50 (m, 4H), 1.76 (m, 2H), 3.03 (m, 1H), 3.21 (m, 2H), 4.32 (m, 2H), 7.40 (s, 1H), 7.66 (m, 1H), 8.08 (s, 1H), 8.10 (m, 1H), 8.30 (s, 1H), 8.69 (d, 1H), 9.05 (d, 1H), 9.45 (s, 1H). | Intermediate 295 and ethyl 5-(4,4,5,5- tetramethyl-1,3,2- dioxaborolan-2- yl)nicotinate |
| 329 | ethyl 4′-(4-cyclopropylthiazol-2-yl)-6′-(3-ethylureido)- 3,3′-bipyridine-5-carboxylate 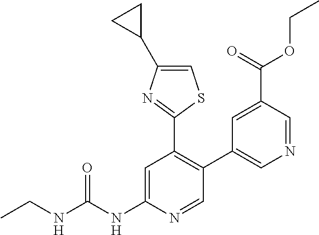 | LC/MS (ES⁺)[(M + H)⁺]: 438 for C₂₂H₂₃N₅O₃S. ¹H NMR (300 MHz, d₆-DMSO): 0.42 (m, 2H), 0.74 (m, 2H), 1.11 (t, 3H), 1.33 (m, 3H), 1.97 (m, 1H), 3.21 (m, 2H), 4.34 (m, 2H), 7.40 (s, 1H), 7.51 (m, 1H), 8.08 (s, 1H), 8.12 (m, 1H), 8.26 (s, 1H), 8.65 (d, 1H), 9.07 (d, 1H), 9.41 (s, 1H). | Intermediate 296 and ethyl 5-(4,4,5,5- tetramethyl-1,3,2- dioxaborolan-2- yl)nicotinate |
| 330 | Ethyl 4′-(4-(2,2-dimethyltetrahydro-2H-pyran-4-yl)thiazol-2-yl)-6′- (3-ethylureido)-3,3′-bipyridine-5-carboxylate 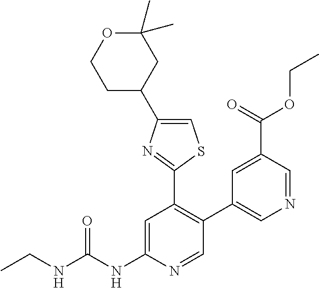 | LC/MS (ES⁺)[(M + H)⁺]: 510 for C₂₆H₃₁N₅O₄S. | Intermediate 298 and ethyl 5-(4,4,5,5- tetramethyl-1,3,2- dioxaborolan-2- yl)nicotinate |
| 331 | methyl 5-(6-(3-ethylureido)-4-(4-(1-methyl-1H-pyrazol-4- yl)thiazol-2-yl)pyridin-3-yl)pyrazine-2-carboxylate 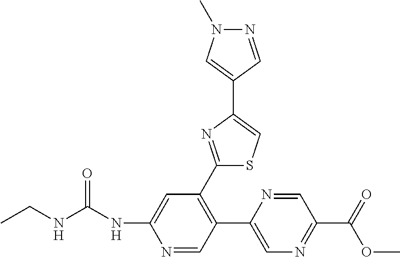 | LC/MS (ES⁺)[(M + H)⁺]: 465 for C₂₁H₂₀N₈O₃S. ¹H NMR (300 MHz, d₆-DMSO): 1.11 (t, 3H), 3.21 (m, 2H), 3.80 (s, 3H), 3.94 (s, 3H), 7.50 (s, 1H), 7.56 (m, 1H), 7.74 (s, 1H), 7.81 (s, 1H), 8.15 (s, 1H), 8.51 (s, 1H), 8.79 (d, 1H), 9.19 (d, 1H), 9.58 (s, 1H). | Intermediate 302 and methyl 5- chloropyrazine-2- carboxylate |
| 332 | methyl 6′-(3-ethylureido)-6-methoxy-4′-(4-(1-methyl-1H- pyrazol-4-yl)thiazol-2-yl)-3,3′-bipyridine-5-carboxylate 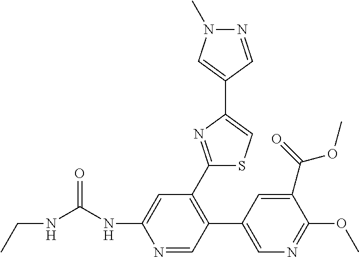 | LC/MS (ES⁺)[(M + H)⁺]: 494 for C₂₃H₂₃N₇O₄S. ¹H NMR (300 MHz, d₆-DMSO): 1.10 (t, 3H), 3.21 (m, 2H), 3.78 (s, 3H), 3.86 (s, 3H), 3.97 (s, 3H), 7.67 (s, 1H), 7.72 (s, 1H), 7.77 (s, 1H), 8.0 (s, 1H), 8.11 (d, 1H), 8.19 (m, 1H), 8.25 (s, 1H), 8.34 (m, 1H), 9.42 (s, 1H). | Intermediate 302 and methyl 5-bromo-2- methoxynicotinate |
| 333 | tetrahydro-2H-pyran-4-yl 6′-(3-ethylureido)-4′-(4-(1-methyl-1H- pyrazol-4-yl)thiazol-2-yl)-2-(tetrahydro-2H-pyran-4-yloxy)-3,3′- bipyridine-5-carboxylate 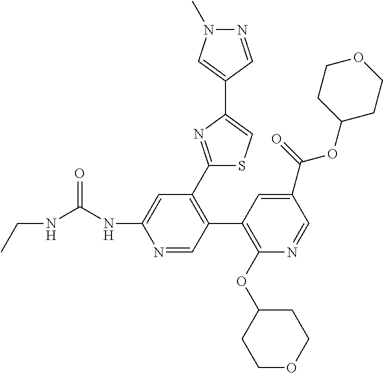 | LC/MS (ES⁺)[(M + H)⁺]: 634 for C₃₁H₃₅N₇O₆S. | Intermediate 281 and Intermediate 302 |
| 334 | methyl 6′-(3-ethylureido)-2-fluoro-4′-(4-(1-methyl-1H- pyrazol-4-yl)thiazol-2-yl)-3,3′-bipyridine-5-carboxylate 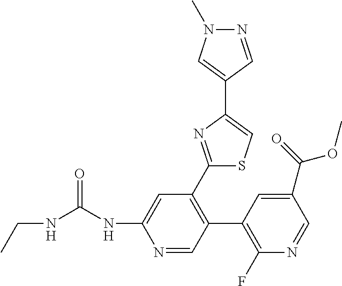 | LC/MS (ES⁺)[(M + H)⁺]: 482 for C₂₂H₂₀FN₇O₃S. | Intermediate 302 and methyl 5-bromo-6- fluoronicotinate |
| 335 | ethyl 6′-(3-ethylureido)-4′-(4-(piperidin-4-yl)thiazol- 2-yl)-3,3′-bipyridine-5-carboxylate 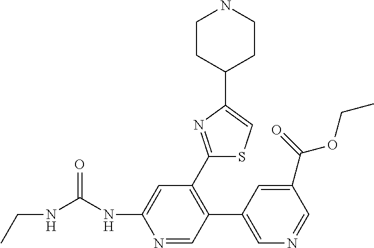 | LC/MS (ES⁺)[(M + H)⁺]: 481 for C₂₄H₂₈N₆O₃S. | Intermediate 297 and ethyl 5-(4,4,5,5- tetramethyl-1,3,2- dioxaborolan-2- yl)nicotinate |

### Intermediate 336

### 6′-(3-ethylureido)-2-(2-(pyrrolidin-1-yl)ethoxy)-4′-(4-(trifluoromethyl)thiazol-2-yl)-3,3′-bipyridine-5-carboxylic acid

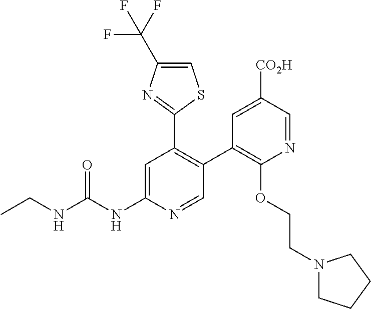

Methyl 6′-(3-ethylureido)-2-fluoro-4′-(4-(trifluoromethyl)thiazol-2-yl)-3,3′-bipyridine-5-carboxylate (Intermediate 162, 200 mg, 0.43 mmol) was added to a THF solution containing sodium hydride (85 mg, 2.13 mmol). The mixture was stirred at room temperature for 18 h. The reaction was neutralized with 2N HCl. The reaction mixture was concentrated to dryness by rotary evaporation. Purified by silica gel flash column chromatography (95:5 CH₂Cl₂/MeOH) gave 220 mg of the title compound.

LC/MS (ES⁺)[(M+H)⁺]: 551 for C₂₄H₂₅F₃N₆O₄S.

### Intermediate 337

cyclopropylmethyl 2-(cyclopropylmethoxy)-6′-(3-ethylureido)-4′-(4-(1-methyl-1H-pyrazol-4-yl)thiazol-2-yl)-3,3′-bipyridine-5-carboxylate

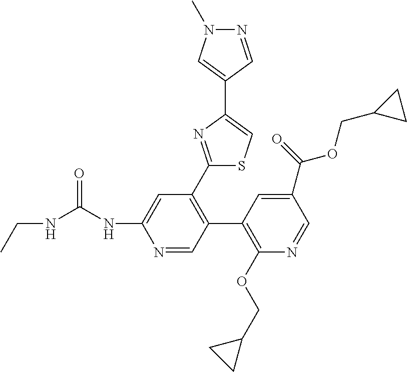

Lithium bis(trimethylsilyl)-amide (0.841 mL, 0.84 mmol) was added to solution of cyclopropylmethanol (0.033 mL, 0.42 mmol) in THF (1.5 mL). Methyl 6′-(3-ethylureido)-2-fluoro-4′-(4-(1-methyl-1H-pyrazol-4-yl)thiazol-2-yl)-3,3′-bipyridine-5-carboxylate (Intermediate 334, 0.081 g, 0.17 mmol) was added after 30 min. The mixture was stirred at room temperature overnight. The reaction was quenched with NH₄OH, partitioned between water and ethyl acetate, the layers were separated, and the organic phase was washed with water and brine, dried over magnesium sulfate, concentrated under reduced pressure, and purified by column chromatography (Silica gel, 0-10% MeOH in CH₂Cl₂) to give 59 mg of crude compound.

LC/MS (ES⁺)[(M+H)⁺]: 574 for C₂₉H₃₁N₇O₄S.

### Intermediates 338-349

The following Intermediates were prepared according to the procedure described for Intermediate 337 using the starting materials indicated in the table.

| Int | Compound | Data | SM |
|---|---|---|---|
| 338 | cyclohexyl 2-(cyclohexyloxy)-6′-(3-ethylureido)-4′-(4- (trifluoromethyl)thiazol-2-yl)-3,3′-bipyridine-5-carboxylate 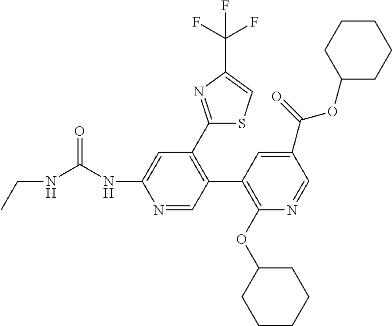 | LC/MS (ES⁺)[(M + H)⁺]: 618 for C₃₀H₃₄F₃N₅O₄S. | Intermediate 162 and cyclohexanol |
| 339 | cyclopropylmethyl 2-(cyclopropylmethoxy)-6′-(3-ethylureido)-4′-(4- (trifluoromethyl)thiazol-2-yl)-3,3′-bipyridine-5-carboxylate 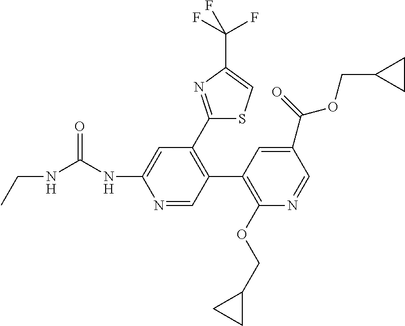 | LC/MS (ES⁺)[(M + H)⁺]: 562 for C₂₆H₂₆F₃N₅O₄S. | Intermediate 162 and cyclopropylmethanol |
| 340 | (1-methylpiperidin-4-yl)methyl 6′-(3-ethylureido)-2-((1-methylpiperidin-4- yl)methoxy)-4′-(4-(trifluoromethyl)thiazol-2-yl)-3,3′-bipyridine-5-carboxylate 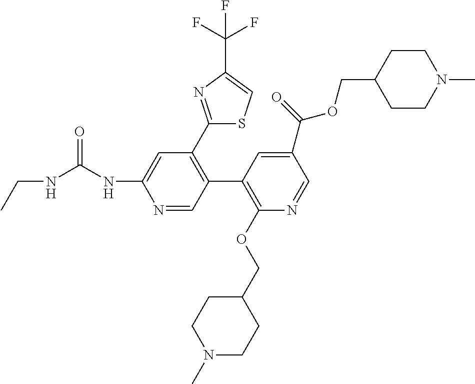 | LC/MS (ES⁺)[(M + H)⁺]: 676 for C₃₂H₄₀F₃N₇O₄S. | Intermediate 162 and (1-methylpiperidin-4- yl)methanol |
| 341 | 1-methylpiperidin-4-yl 6′-(3-ethylureido)-2-(1-methylpiperidin-4- yloxy)-4′-(4-(trifluoromethyl)thiazol-2-yl)-3,3′-bipyridine-5-carboxylate 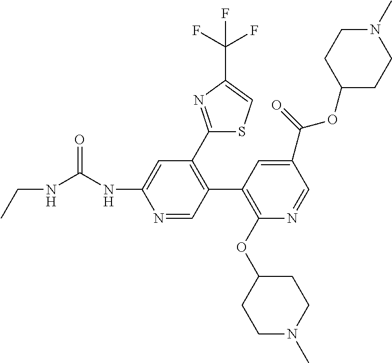 | LC/MS (ES⁺)[(M + H)⁺]: 648 for C₃₀H₃₆F₃N₇O₄S. | Intermediate 162 and 1-methylpiperidin-4-ol |
| 342 | cyclopentyl 2-(cyclopentyloxy)-6′-(3-ethylureido)-4′-(4- (trifluoromethyl)thiazol-2-yl)-3,3′-bipyridine-5-carboxylate 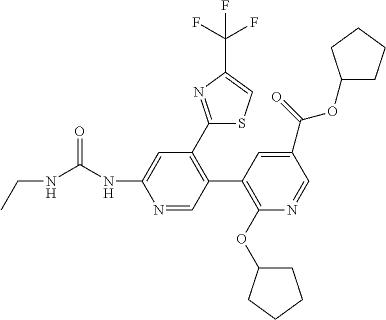 | LC/MS (ES⁺)[(M + H)⁺]: 590 for C₂₈H₃₀F₃N₅O₄S. | Intermediate 162 and cyclopentanol |
| 343 | 1-isopropylpiperidin-4-yl 6′-(3-ethylureido)-2-(1-isopropylpiperidin- 4-yloxy)-4′-(4-(trifluoromethyl)thiazol-2-yl)-3,3′-bipyridine-5-carboxylate 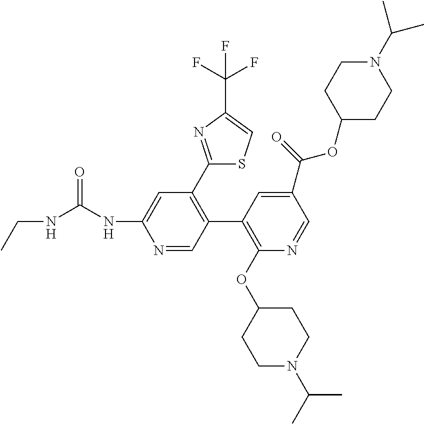 | LC/MS (ES⁺)[(M + H)⁺]: 704 for C₃₄H₄₄F₃N₇O₄S. | Intermediate 162 and 1-isopropylpiperidin- 4-ol |
| 344 | 1,2,2,6,6-pentamethylpiperidin-4-yl 6′-(3-ethylureido)-2-(1,2,2,6,6- pentamethylpiperidin-4-yloxy)-4′-(4-(trifluoromethyl)thiazol-2- yl)-3,3′-bipyridine-5-carboxylate 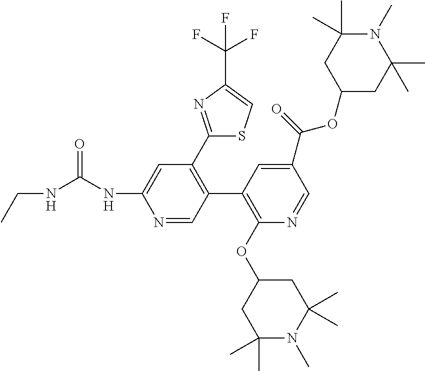 | LC/MS (ES⁺)[(M + H)⁺]: 760 for C₃₈H₅₂F₃N₇O₄S. | Intermediate 162 and 1,2,2,6,6- pentamethylpiperidin- 4-ol |
| 345 | 3-cyclopentylpropyl 2-(3-cyclopentylpropoxy)-6′-(3-ethylureido)-4′-(4- (trifluoromethyl)thiazol-2-yl)-3,3′-bipyridine-5-carboxylate 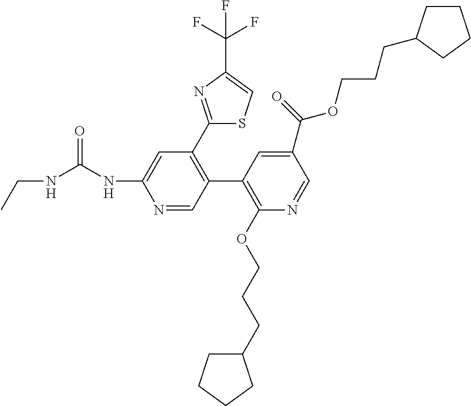 | LC/MS (ES⁺)[(M + H)⁺]: 674 for C₃₄H₄₂F₃N₅O₄S. | Intermediate 162 and 3-cyclopentylpropan- 1-ol |
| 346 | 2-(1-methylpyrrolidin-2-yl)ethyl 6′-(3-ethylureido)-2-(2-(1-methylpyrrolidin-2- yl)ethoxy)-4′-(4-(trifluoromethyl)thiazol-2-yl)-3,3′-bipyridine-5-carboxylate 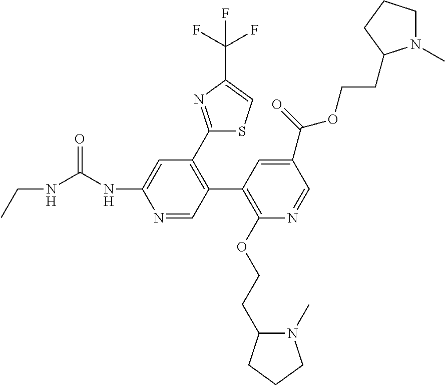 | LC/MS (ES⁺)[(M + H)⁺]: 676 for C₃₂H₄₀F₃N₅O₄S. | Intermediate 162 and 2-(1- methylpyrrolidin-2- yl)ethanol |
| 347 | 6′-(3-ethylureido)-2-((S)-2-hydroxypropoxy)-4′-(4-(trifluoromethyl)thiazol-2- yl)-3,3′-bipyridine-5-carboxylic acid 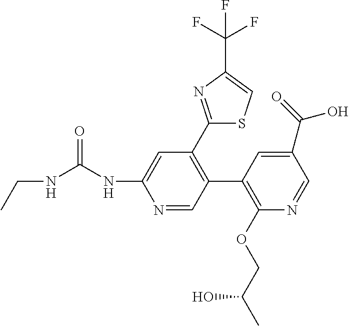 | LC/MS (ES⁺)[(M + H)⁺]: 512 for C₂₁H₂₀F₃N₅O₄S. | Intermediate 162 and (S)-propane-1,2-diol |
| 348 | 6′-(3-ethylureido)-2-((R)-2-hydroxypropoxy)-4′-(4-(trifluoromethyl)thiazol-2- yl)-3,3′-bipyridine-5-carboxylic acid 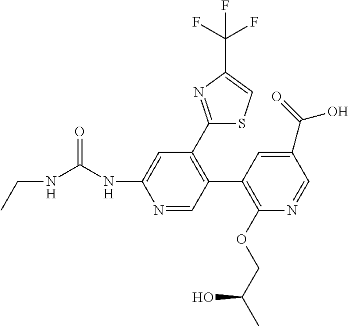 | LC/MS (ES⁺)[(M + H)⁺]: 512 for C₂₁H₂₀F₃N₅O₄S. | Intermediate 162 and (R)-propane-1,2-diol |
| 349 | tert-butyl 5-bromo-6-((1R,3r,5S)-8-methyl-8-azabicyclo[3.2.1]octan- 3-yloxy)nicotinate 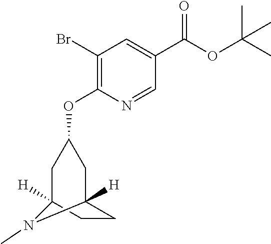 | LC/MS (ES⁺)[(M + H)⁺]: 397, 399 for C₁₈H₂₅BrN₂O₃. | tert-butyl 5-bromo-6- fluoronicotinate and (1R,3r,5S)-8-methyl- 8-azabicyclo[3.2.1]octan- 3-ol |

### Intermediates 350-386

The following Intermediates were prepared according to the procedure described Intermediate 9 using the starting material indicated in the table.

| Int | Compound | Data | SM |
|---|---|---|---|
| 350 | methyl 6-(3-ethylureido)-4-(4-(trifluoromethyl)thiazol-2- yl)-3,4′-bipyridine-2′-carboxylate 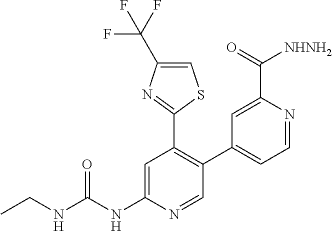 | LC/MS (ES⁺)[(M + H)⁺]: 452 for C₁₈H₁₆F₃N₇O₂S. ¹H NMR (300 MHz, d₆-DMSO): 1.11 (t, 3H), 3.21 (m, 2H), 4.57 (s, 2H), 7.52 (m, 2H), 7.82 (s, 1H), 8.16 (s, 1H), 8.37 (s, 1H), 8.58 (s, 1H), 8.60 (d, 1H), 9.51 (s, 1H), 9.91 (s, 1H). | Intermediate 305 |
| 351 | 1-ethyl-3-(5-(4-(hydrazinecarbonyl)thiazol-2-yl)-4-(4- (trifluoromethyl)thiazol-2-yl)pyridin-2-yl)urea 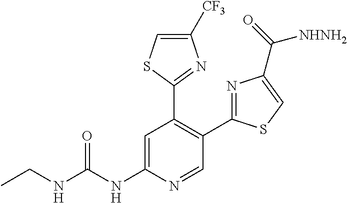 | LC/MS (ES⁺)[(M + H)⁺]: 458 for C₁₆H₁₄F₃N₇O₂S₂. | Intermediate 306 |
| 352 | 1-ethyl-3-(5′-(hydrazinecarbonyl)-6′-methoxy-4-(4- (trifluoromethyl)thiazol-2-yl)-3,3′-bipyridin-6-yl)urea 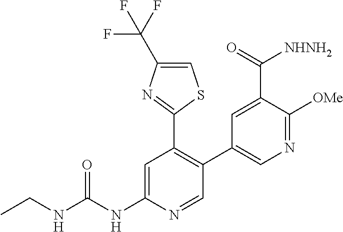 | LC/MS (ES⁺)[(M + H)⁺]: 482 for C₁₉H₁₈F₃N₇O₃S. | Intermediate 307 |
| 353 | 1-ethyl-3-(5-(5-(hydrazinecarbonyl)pyrazin-2-yl)-4-(4- (trifluoromethyl)thiazol-2-yl)pyridin-2-yl)urea 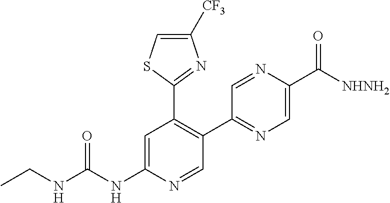 | LC/MS (ES⁺)[(M + H)⁺]: 453 for Cₗ₇H₁₅F₃N₈O₂S. ¹H NMR (300 MHz, d₆-DMSO): 1.11 (t, 3H), 3.22 (m, 2H), 4.66 (m, 2H), 7.45 (t, 1H), 8.15 (s, 1H), 8.59 (s, 1H), 8.61 (m, 1H), 8.76 (d, 1H), 9.04 (d, 1H), 9.62 (s, 1H), 10.18 (s, 1H). | Intermediate 308 |
| 354 | 1-ethyl-3-(2′-(hydrazinecarbonyl)-4-(5-phenyl-1,3,4- oxadiazol-2-yl)-3,4′-bipyridin-6-yl)urea 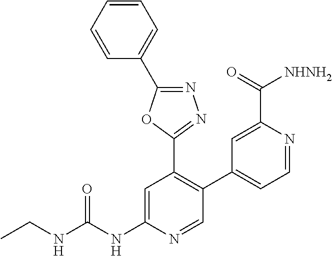 | LC/MS (ES⁺)[(M + H)⁺]: 445 for C₂₂H₂₀N₈O₃. | Intermediate 310 |
| 355 | 1-ethyl-3-(5-(4-(hydrazinecarbonyl)thiazol-2-yl)-4-(5-phenyl- 1,3,4-oxadiazol-2-yl)pyridin-2-yl)urea 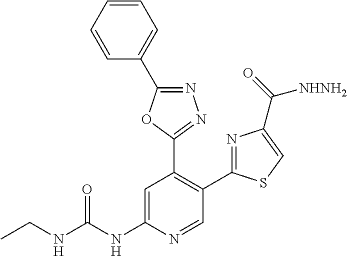 | LC/MS (ES⁺)[(M + H)⁺]: 451 for C₂₀H₁₈N₈O₃S. | Intermediate 313 |
| 356 | 1-ethyl-3-(5-(5-(hydrazinecarbonyl)thiazol-2-yl)-4-(5-phenyl- 1,3,4-oxadiazol-2-yl)pyridin-2-yl)urea 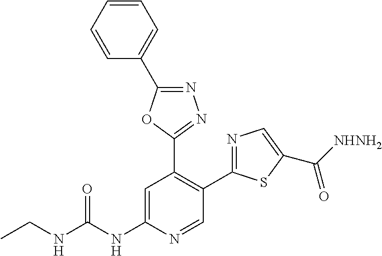 | LC/MS (ES⁺)[(M + H)⁺]: 451 for C₂₀H₁₈N₈O₃S. | Intermediate 312 |
| 357 | 1-ethyl-3-(5-(5-(hydrazinecarbonyl)pyrazin-2-yl)-4-(5-phenyl- 1,3,4-oxadiazol-2-yl)pyridin-2-yl)urea 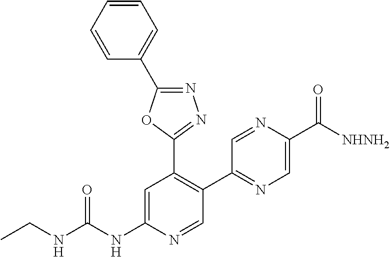 | LC/MS (ES⁺)[(M + H)⁺]: 446 for C₂₁H₁₉N₉O₃ | Intermediate 314 |
| 358 | 1-ethyl-3-(5′-(hydrazinecarbonyl)-4-(5-phenyl-1,3,4- oxadiazol-2-yl)-3,3′-bipyridin-6-yl)urea 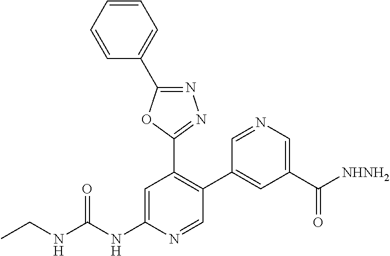 | LC/MS (ES⁺)[(M + H)⁺]: 455 for C₂₂H₂₀N₈O₃. | Intermediate 311 |
| 359 | 1-ethyl-3-(5′-(hydrazinecarbonyl)-6′-methoxy-4-(5-phenyl- 1,3,4-oxadiazol-2-yl)-3,3′-bipyridin-6-yl)urea 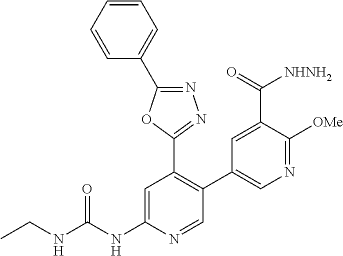 | LC/MS (ES⁺)[(M + H)⁺]: 475 for C₂₃H₂₂N₈O_{4.} | Intermediate 315 |
| 360 | 1-ethyl-3-(4-(5-(4-fluorophenyl)-1,3,4-oxadiazol-2-yl)-5′- (hydrazinecarbonyl)-3,3′-bipyridin-6-yl)urea 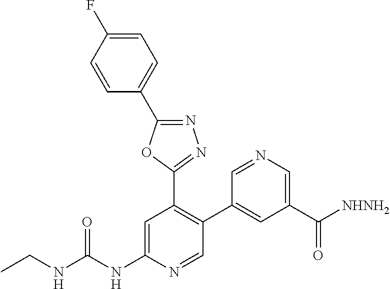 | LC/MS (ES⁺)[(M + H)⁺]: 463 for C₂₂H₁₉FN₈O₃ | Intermediate 316 |
| 361 | 1-ethyl-3-(5-(5-(hydrazinecarbonyl)-4-(pyrimidin-2- yl)thiazol-2-yl)-4-(4-phenylthiazol-2-yl)pyridin-2-yl)urea 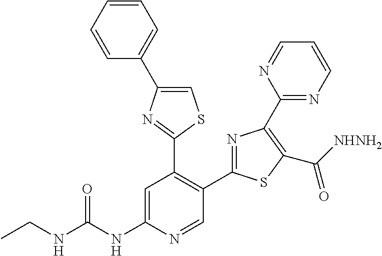 | LC/MS (ES⁺)[(M + H)⁺]: 544 for C₂₅H₂₁N₉O₂S₂. | Intermediate 317 |
| 362 | 1-ethyl-3-(5-(5-(hydrazinecarbonyl)-4-(1-methyl-1H-1,2,4-triazol-5- yl)thiazol-2-yl)-4-(4-phenylthiazol-2-yl)pyridin-2-yl)urea 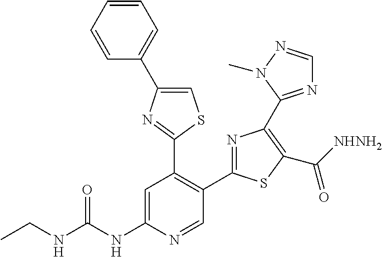 | LC/MS (ES⁺)[(M + H)⁺]: 547 for C₂₄H₂₂N₁₀O₂S₂. | Intermediate 318 |
| 363 | 1-ethyl-3-(5-(5-(hydrazinecarbonyl)-4-(pyrimidin-2-yl)thiazol-2-yl)- 4-(4-(pyridin-2-yl)thiazol-2-yl)pyridin-2-yl)urea 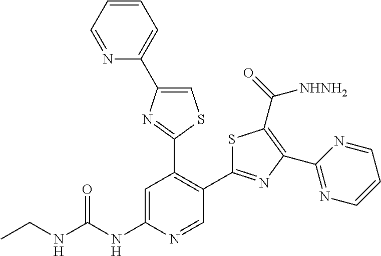 | LC/MS (ES⁺)[(M + H)⁺]: 545 for C₂₄H₂₀N₁₀O₂S₂. | Intermediate 299 |
| 364 | 1-ethyl-3-(5′-(hydrazinecarbonyl)-4- (4-(6-methoxypyridin-2-yl)thiazol-2-yl)-3,3′-bipyridin-6-yl)urea 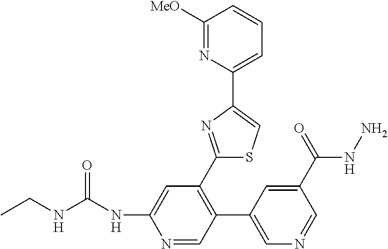 | LC/MS (ES⁺)[(M + H)⁺]: 491 for C₂₃H₂₂N₈O₃S. | Intermediate 325 |
| 365 | 1-ethyl-3-(5-(5-(hydrazinecarbonyl)pyrazin-2- yl)-4-(4-(1-methyl-1H-pyrazol-4-yl)thiazol-2-yl)pyridin-2-yl)urea 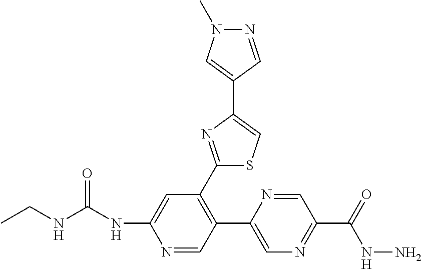 | LC/MS (ES⁺)[(M + H)⁺]: 465 for C₂₀H₂₀N₁₀O₂S. | Intermediate 331 |
| 366 | 1-ethyl-3-(5′-(hydrazinecarbonyl)-4-(4-(1-methyl-1H-pyrazol-4- yl)thiazol-2-yl)-2′-(tetrahydro-2H-pyran-4-yloxy)-3,3′-bipyridin-6-yl)urea 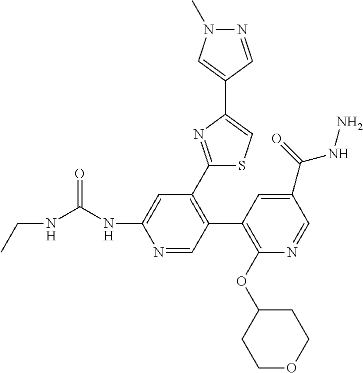 | LC/MS (ES⁺)[(M + H)⁺]: 564 for C₂₆H₂₉N₉O₄S. | Intermediate 333 |
| 367 | 1-(2′-(cyclopropylmethoxy)-5′-(hydrazinecarbonyl)-4-(4-(1- methyl-1H-pyrazol-4-yl)thiazol-2-yl)-3,3′-bipyridin-6-yl)-3-ethylurea 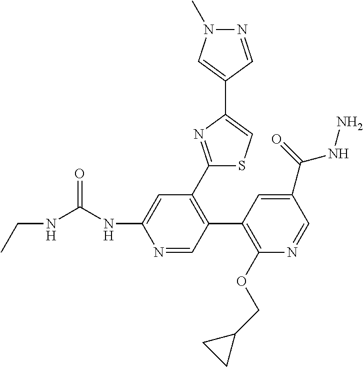 | LC/MS (ES⁺)[(M + H)⁺]: 534 for C₂₅H₂₇N₉O₃S | Intermediate 337 |
| 368 | 1-ethyl-3-(5′-(hydrazinecarbonyl)-6′-methoxy-4-(4-(1-methyl- 1H-pyrazol-4-yl)thiazol-2-yl)-3,3′-bipyridin-6-yl)urea 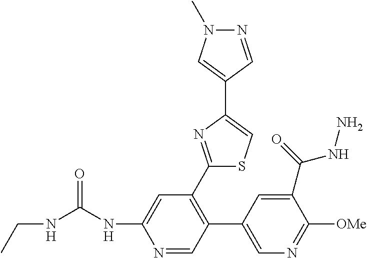 | LC/MS (ES⁺)[(M + H)⁺]: 494 for C₂₂H₂₃N₉O₃S. ¹H NMR (300 MHz, d₆-DMSO): 1.11 (t, 3H), 3.19 (m, 2H), 3.87 (s, 3H), 4.00 (s, 3H), 4.6 (s, 2H), 7.68 (m, 1H), 7.75 (s, 1H), 7.76 (s, 1H), 8.03 (s, 1H), 8.05 (m, 1H), 8.18 (s, 1H), 8.23 (s, 1H), 8.24 (m, 1H), 8.98 (d, 1H), 9.41 (s, 1H). | Intermediate 332 |
| 369 | 1-ethyl-3-(5′-(hydrazinecarbonyl)-4-(4-(6-methoxypyridin- 3-yl)thiazol-2-yl)-3,3′-bipyridin-6-yl)urea 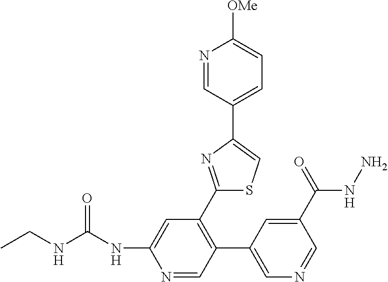 | LC/MS (ES⁺)[(M + H)⁺]: 491 for C₂₃H₂₂N₈O₃S. | Intermediate 326 |
| 370 | 1-ethyl-3-(5′-(hydrazinecarbonyl)-6′-methoxy-4-(4-(2-(2- methoxyethoxy)pyridin-3-yl)thiazol-2-yl)-3,3′-bipyridin-6-yl)urea 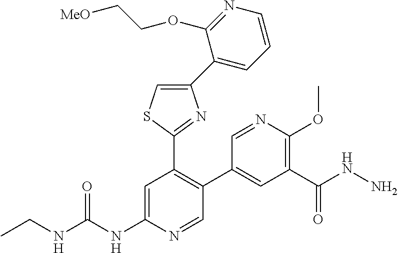 | LC/MS (ES⁺)[(M + H)⁺]: 565 for C₂₆H₂₈N₈O₅S. | Intermediate 322 |
| 371 | 1-ethyl-3-(5′-(hydrazinecarbonyl)-4-(4-(2-(2- methoxyethoxy)pyridin-3-yl)thiazol-2-yl)-3,3′-bipyridin-6-yl)urea 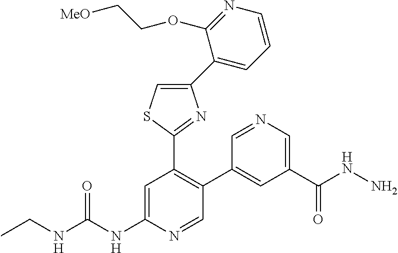 | LC/MS (ES⁺)[(M + H)⁺]: 535 for C₂₅H₂₆N₈O₄S. | Intermediate 323 |
| 372 | 1-ethyl-3-(5′-(hydrazinecarbonyl)-4-(4-(2-(2-methoxyethoxy)pyridin-3- yl)thiazol-2-yl)-2′-(tetrahydro-2H-pyran-4-yloxy)-3,3′-bipyridin-6-yl)urea 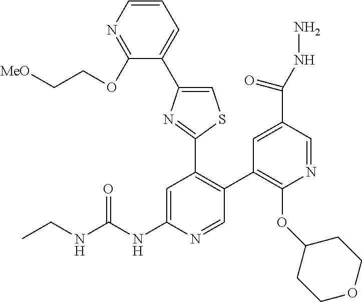 | LC/MS (ES⁺)[(M + H)⁺]: 635 for C₃₀H₃₄N₈O₆S | Intermediate 324 |
| 373 | 1-(4-(4-(2,2-dimethyltetrahydro-2H-pyran-4-yl)thiazol-2- yl)-5′-(hydrazinecarbonyl)-3,3′-bipyridin-6-yl)-3-ethylurea 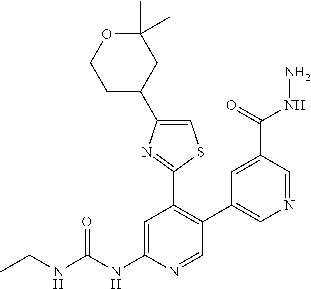 | LC/MS (ES⁺)[(M + H)⁺]: 496 for C₂₄H₂₉N₇O₃S. | Intermediate 330 |
| 374 | 1-(4-(4-cyclohexylthiazol-2-yl)-5′-(hydrazinecarbonyl)- 3,3′-bipyridin-6-yl)-3-ethylurea 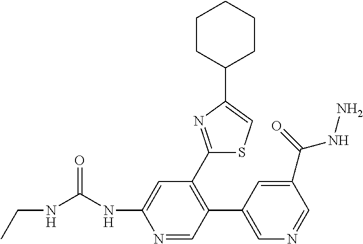 | LC/MS (ES⁺)[(M + H)⁺]: 466 for C₂₃H₂₇N₇O₂S. | Intermediate 327 |
| 375 | 1-(4-(4-cyclopentylthiazol-2-yl)-5′- (hydrazinecarbonyl)-3,3′-bipyridin-6-yl)-3-ethylurea 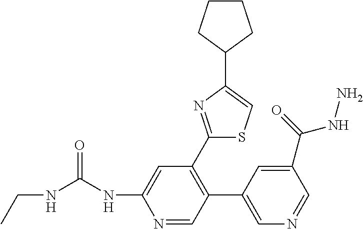 | LC/MS (ES⁺)[(M + H)⁺]: 452 for C₂₂H₂₅N₇O₂S. ¹H NMR (300 MHz, d₆-DMSO): 1.10 (t, 3H), 1.40 (m, 2H), 1.52 (m, 4H), 1.81 (m, 2H), 3.06 (m, 1H), 3.20 (m, 2H), 4.56 (s, 2H), 7.39 (s, 1H), 7.66 (m, 1H), 8.07 (m, 1H), 8.10 (s, 1H), 8.28 (s, 1H), 8.51 (d, 1H), 8.94 (d, 1H), 9.43 (s, 1H), 9.95 (s, 1H). | Intermediate 328 |
| 376 | 1-(4-(4-cyclopropylthiazol-2-yl)-5′- (hydrazinecarbonyl)-3,3′-bipyridin-6-yl)-3-ethylurea 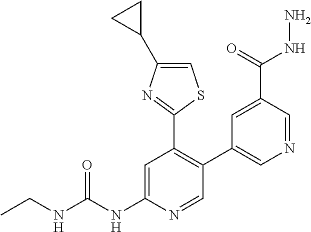 | LC/MS (ES⁺)[(M + H)⁺]: 424 for C₂₀H₂₁N₇O₂S. | Intermediate 329 |
| 377 | 1-ethyl-3-(5′-(hydrazinecarbonyl)-4-(4-(piperidin-4- yl)thiazol-2-yl)-3,3′-bipyridin-6-yl)urea 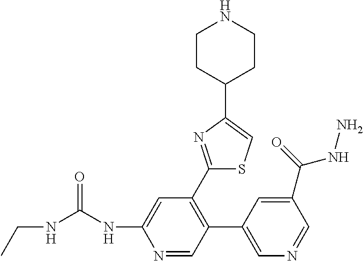 | LC/MS (ES⁺)[(M + H)⁺]: 467 for C₂₂H₂₆N₈O₂S. | Intermediate 335 |
| 378 | 1-(2′-(cyclohexyloxy)-5′-(hydrazinecarbonyl)-4-(4- (trifluoromethyl)thiazol-2-yl)-3,3′-bipyridin-6-yl)-3-ethylurea 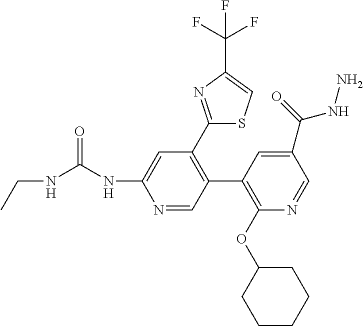 | LC/MS (ES⁺)[(M + H)⁺]: 467 for C₂₄H₂₆F₃N₇O₃S. | Intermediate 338 |
| 379 | 1-(2′-(cyclopentyloxy)-5′-(hydrazinecarbonyl)-4-(4- (trifluoromethyl)thiazol-2-yl)-3,3′-bipyridin-6-yl)-3-ethylurea 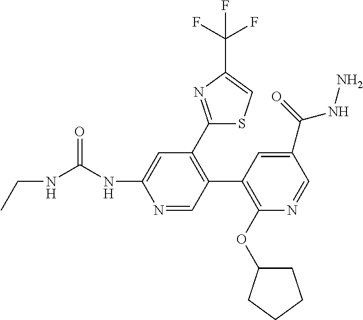 | LC/MS (ES⁺)[(M + H)⁺]: 536 for C₂₃H₂₄F₃N₇O₃S. | Intermediate 342 |
| 380 | 1-ethyl-3-(5′-(hydrazinecarbonyl)-2′-(1-methylpiperidin-4-yloxy)-4-(4- (trifluoromethyl)thiazol-2-yl)-3,3′-bipyridin-6-yl)urea 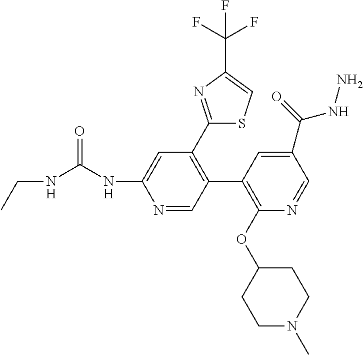 | LC/MS (ES⁺)[(M + H)⁺]: 565 for C₂₄H₂₇F₃N₈O₃S. | Intermediate 341 |
| 381 | 1-ethyl-3-(5′-(hydrazinecarbonyl)-2′-(1-isopropylpiperidin- 4-yloxy)-4-(4-(trifluoromethyl)thiazol-2-yl)-3,3′-bipyridin-6-yl)urea 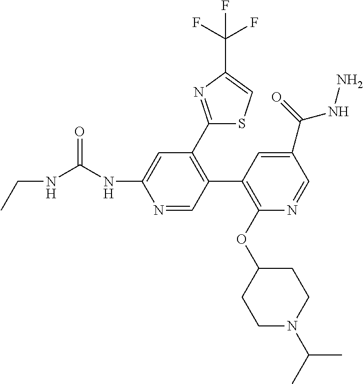 | LC/MS (ES⁺)[(M + H)⁺]: 593 for C₂₆H₃₁F₃N₈O₃S. | Intermediate 343 |
| 382 | 1-ethyl-3-(5′-(hydrazinecarbonyl)-2′-(1,2,2,6,6-pentamethylpiperidin- 4-yloxy)-4-(4-(trifluoromethyl)thiazol-2-yl)-3,3′-bipyridin-6-yl)urea 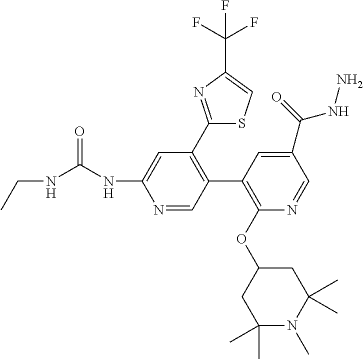 | LC/MS (ES⁺)[(M + H)⁺]: 621 for C₂₈H₃₅F₃N₈O₃S. | Intermediate 344 |
| 383 | 1-(2′-(cyclopropylmethoxy)-5′-(hydrazinecarbonyl)-4-(4- (trifluoromethyl)thiazol-2-yl)-3,3′-bipyridin-6-yl)-3-ethylurea 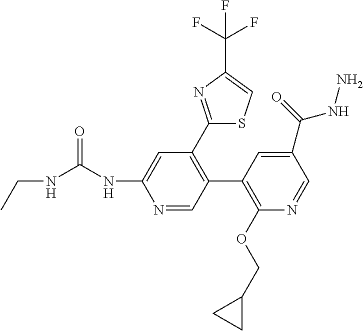 | LC/MS (ES⁺)[(M + H)⁺]: 522 for C₂₂H₂₂F₃N₇O₃S. | Intermediate 339 |
| 384 | 1-(2′-(3-cyclopentylpropoxy)-5′-(hydrazinecarbonyl)-4-(4- (trifluoromethyl)thiazol-2-yl)-3,3′-bipyridin-6-yl)-3-ethylurea 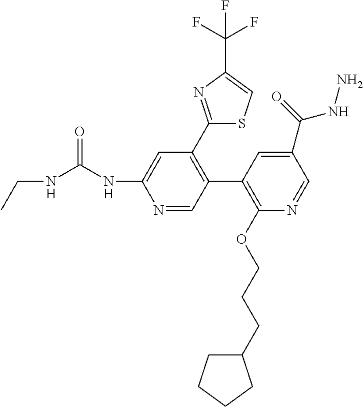 | LC/MS (ES⁺)[(M + H)⁺]: 578 for C₂₆H₃₀F₃N₇O₃S. | Intermediate 345 |
| 385 | 1-ethyl-3-(5′-(hydrazinecarbonyl)-2′-(2-(1-methylpyrrolidin- 2-yl)ethoxy)-4-(4-(trifluoromethyl)thiazol-2-yl)-3,3′-bipyridin-6-yl)urea 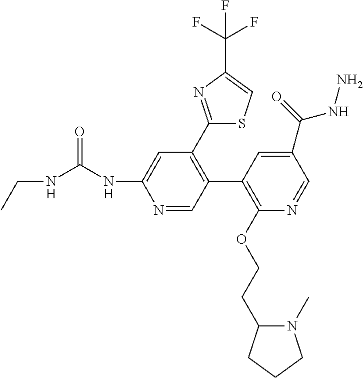 | LC/MS (ES⁺)[(M + H)⁺]: 579 for C₂₅H₂₉F₃N₈O₃S. | Intermediate 346 |
| 386 | 1-ethyl-3-(5′-(hydrazinecarbonyl)-2′-((1-methylpiperidin-4-yl)methoxy)- 4-(4-(trifluoromethyl)thiazol-2-yl)-3,3′-bipyridin-6-yl)urea 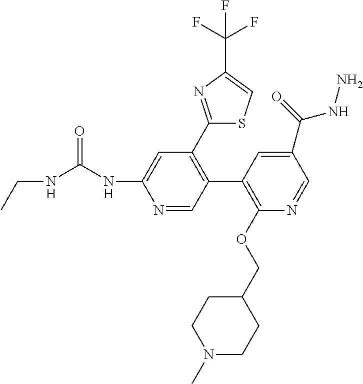 | LC/MS (ES⁺)[(M + H)⁺]: 579 for C₂₅H₂₉F₃N₈O₃S. | Intermediate 340 |

### Intermediate 387

### 1-ethyl-3-(5′-(hydrazinecarbonyl)-2′-(2-(pyrrolidin-1-yl)ethoxy)-4-(4-(trifluoromethyl)thiazol-2-yl)-3,3′-bipyridin-6-yl)urea

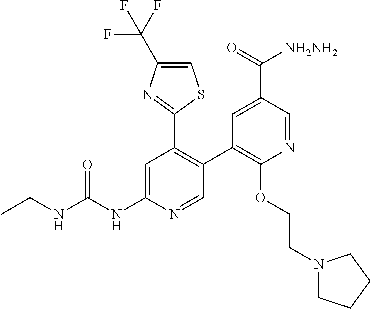

6′-(3-ethylureido)-2-(2-(pyrrolidin-1-yl)ethoxy)-4′-(4-(trifluoromethyl)thiazol-2-yl)-3,3′-bipyridine-5-carboxylic acid (Intermediate 336, 220 mg, 0.40 mmol) was dissolved in a DMF solution containing HATU (152 mg, 0.40 mmol) and diisopropylethyl amine (0.139 mL, 0.80 mmol). The solution was stirred for 30 minutes. Hydrazine (0.015 mL, 0.48 mmol) was added in a single portion. The reaction mixture was stirred for 0.5 hour. The reaction mixture was diluted with EtOAc, washed with water and brine, dried over Na₂SO₄, filtered and concentrated to give the title compound which was used without further purification.

LC/MS (ES⁺)[(M+H)⁺]: 565 for C₂₄H₂₇F₃N₈O₃S.

### Intermediates 388-391

The following Intermediates were prepared according to the synthesis described for Intermediate 387 using the starting materials indicated in the table.

| Int | Compound | Data | SM |
|---|---|---|---|
| 388 | 1-ethyl-3-(5-(6-(hydrazinecarbonyl)pyridazin-3-yl)-4-(4-(tri- fluoromethyl)thiazol-2-yl)pyridin-2-yl)urea 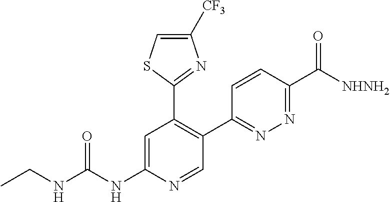 | LC/MS (ES⁺) [(M + H)⁺]: 453 for C₁₇H₁₅F₃N₈O₂S. | Intermediate 309 |
| 389 | 1-ethyl-3-(4-(4-(2-fluoropyridin-3-yl)thiazol-2-yl)-5′-(hydra- zinecarbonyl)-3,3′-bipyridin-6-yl)urea 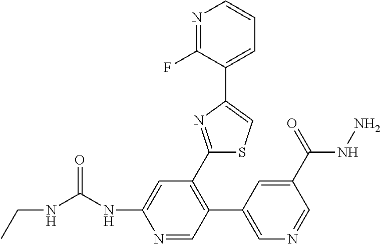 | LC/MS (ES⁺) [(M + H)⁺]: 479 for C₂₂H₁₉F₃N₈O₂S. | Intermediate 284 |
| 390 | 1-ethyl-3-(5′-(hydrazinecarbonyl)-2′-((R)-2-hydroxy- propoxy)-4-(4-(trifluoromethyl)thiazol-2-yl)-3,3′- bipyridin-6-yl)urea 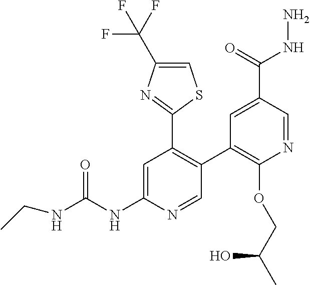 | LC/MS (ES⁺) [(M + H)⁺]: 526 for C₂₁H₂₂F₃N₇O₄S. | Intermediate 348 |
| 391 | 1-ethyl-3-(5′-(hydrazinecarbonyl)-2′-((S)-2-hydroxy- propoxy)-4-(4-(trifluoromethyl)thiazol-2-yl)-3,3′- bipyridin-6-yl)urea 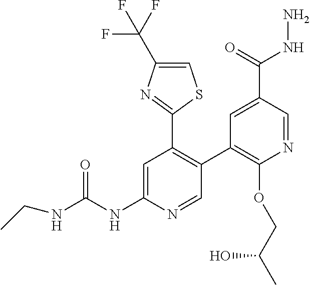 | LC/MS (ES⁺) [(M + H)⁺]: 526 for C₂₁H₂₂F₃N₇O₄S. | Intermediate 347 |

### Intermediate 392

### (S)-tert-butyl 1-cyclohexyl-2-(2-(6′-(3-ethylureido)-4′-(4-(trifluoromethyl)thiazol-2-yl)-3,3′-bipyridine-5-carbonyl)hydrazinyl)-2-oxoethylcarbamate

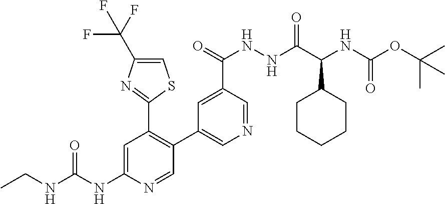

In a glass vial, 1-ethyl-3-(5′-(hydrazinecarbonyl)-4-(4-(trifluoromethyl)thiazol-2-yl)-3,3′-bipyridin-6-yl)urea (Intermediate 9, 300 mg, 0.66 mmol) and (S)-2-(tert-butoxycarbonylamino)-2-cyclohexylacetic acid (188 mg, 0.73 mmol) were combined and dissolved in a DMF solution containing diisopropylethyl amine (0.173 mL, 1.00 mmol). The reaction mixture was stirred for 5 min, then HATU (329 mg, 0.86 mmol) was added in a single portion. The reaction mixture was diluted with EtOAc, washed with water and brine, dried over Na₂SO₄, filtered and concentrated to a residue which was purified by silica gel flash column chromatography (95:5 CH₂Cl₂/MeOH).

LC/MS (ES⁺)(M+H)⁺: 691 for C₃₁H₃₇F₃N₈O₅S.

### Intermediates 393-402

The following Intermediates were prepared according to the procedure described for Intermediate 392 using the starting materials indicated in the table.

| Int | Compound | Data | SM |
|---|---|---|---|
| 393 | (S)-tert-butyl 3-(2-(6′-(3-ethylureido)-4′-(4-(trifluoromethyl)thiazol-2-yl)- 3,3′-bipyridine-5-carbonyl)hydrazinecarbonyl)morpholine-4-carboxylate 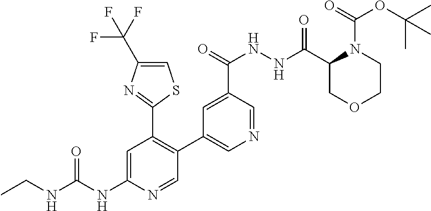 | LC/MS (ES⁺) [(M + H)⁺]: 665 for C₂₈H₃₁F₃N₈O₆S. | Intermediate 9 and (S)-4-(tert- butoxycarbonyl) morpholine-3- carboxylic acid |
| 394 | (R)-tert-butyl 3-(2-(6′-(3-ethylureido)-4′-(4-(trifluoromethyl)thiazol-2-yl)-3,3′-bi- pyridine-5-carbonyl)hydrazinecarbonyl)morpholine-4-carboxylate 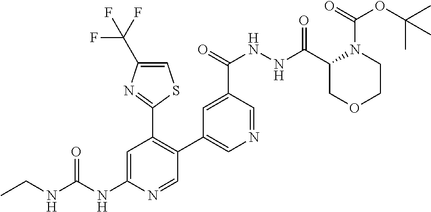 | LC/MS (ES⁺) [(M + H)⁺]: 665 for C₂₈H₃₁F₃N₈O₆S. | Intermediate 9 and (R)-4-(tert- butoxycarbonyl) morpholine-3- carboxylic acid |
| 395 | (S)-tert-butyl 1-cyclohexyl-2-(2-(6-(3-ethylureido)-4-(4-(trifluoromethyl)thiazol-2-yl)- 3,4′-bipyridine-2′-carbonyl)hydrazinyl)-2-oxoethylcarbamate 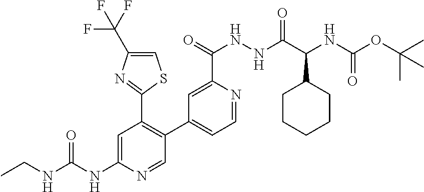 | LC/MS (ES⁺) [(M + H)⁺]: 691 for C₃₁H₃₇F₃N₈O₅S. | Intermediate 350 and (S)-2-(tert- butoxycarbonyl- amino)-2- cyclohexylacetic acid |
| 396 | (S)-tert-butyl 3-(2-(6-(3-ethylureido)-4-(4-(trifluoromethyl)thiazol-2-yl)-3,4′- bipyridine-2′-carbonyl)hydrazinecarbonyl)morpholine-4-carboxylate 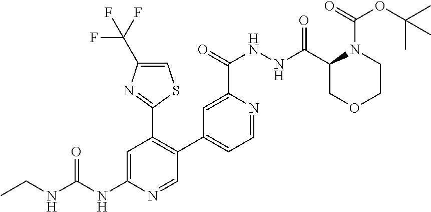 | LC/MS (ES⁺) [(M + H)⁺]: 665 for C₂₈H₃₁F₃N₈O₆S. | Intermediate 350 and (S)-2-(tert- butoxycarbonyl) morpholine-3- carboxylic acid |
| 397 | (R)-tert-butyl 3-(2-(6-(3-ethylureido)-4-(4-(trifluoromethyl)thiazol-2-yl)-3,4′- bipyridine-2′-carbonyl)hydrazinecarbonyl)morpholine-4-carboxylate 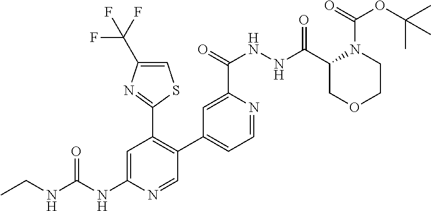 | LC/MS (ES⁺) [(M + H)⁺]: 665 for C₂₈H₃₁F₃N₈O₆S. | Intermediate 350 and (R)-4-(tert- butoxycarbonyl) morpholine-3- carboxylic acid |
| 398 | (S)-tert-butyl 1-(2-(6-(3-ethylureido)-4-(4-(trifluoromethyl)thiazol-2-yl)-3,4′- bipyridine-2′-carbonyl)hydrazinyl)-3-methyl-1-oxobutan-2-yl(methyl) carbamate 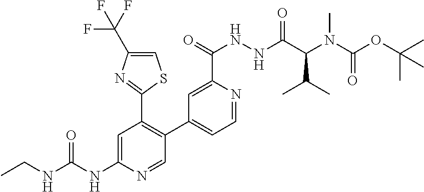 | LC/MS (ES⁺) [(M + H)⁺]: 665 for C₂₉H₃₅F₃N₈O₅S.. | Intermediate 350 and (S)-2-(tert- butoxycarbonyl) methyl)amino)-3- methylbutanoic acid |
| 399 | 1-(2′-(2-acetylhydrazinecarbonyl)-4-(4-(trifluoromethyl)thiazol-2-yl)- 3,4′-bipyridin-6-yl)-3-ethylurea 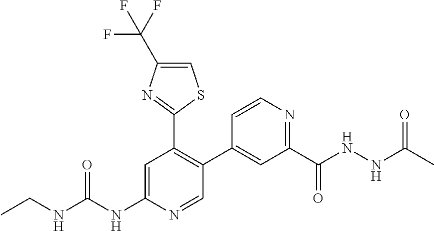 | LC/MS (ES⁺) [(M + H)⁺]: 494 for C₂₀H₁₈F₃N₇O₃S. | Intermediate 50 and acetohydrazide |
| 400 | 1-(2′-(2-acetylhydrazinecarbonyl)-4-(5-phenyl-1,3,4-oxadiazol- 2-yl)-3,4′-bipyridin-6-yl)-3-ethylurea 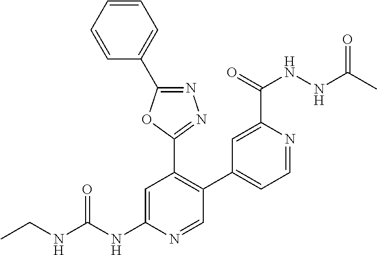 | LC/MS (ES⁺) [(M + H)⁺]: 487 for C₂₄H₂₂N₈O₄. ¹H NMR (300 MHz, d₆-DMSO): 1.11 (t, 3H), 1.91 (s, 3H), 3.21 (m, 2H), 7.60 (m, 4H), 7.69 (m, 3H), 8.07 (s, 1H), 8.42 (s, 1H), 8.53 (s, 1H), 8.71 (d, 1H), 9.77 (s, 1H), 10.08 (s, 1H), 10.51 (s, 1H). | Intermediate 282 and acetohydrazide |
| 401 | 1-(5-(5-(2-acetylhydrazinecarbonyl)thiazol-2-yl)-4-(5-phenyl-1,3,4- oxadiazol-2-yl)pyridin-2-yl)-3-ethylurea 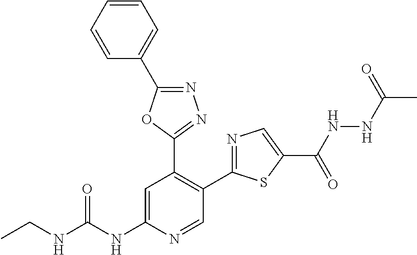 | LC/MS (ES⁺) [(M + H)⁺]: 493 for C₂₂H₂₀N₈O₄S. ¹H NMR (300 MHz, d₆-DMSO): 1.10 (t, 3H), 1.92 (s, 3H), 3.20 (m, 2H), 7.63 (m, 2H), 7.71 (t, 1H), 7.82 (m, 2H), 8.37 (s, 1H), 8.44 (s, 1H), 8.76 (s, 1H), 9.95 (s, 1H), 10.06 (s, 1H), 10.64 (s, 1H). | Intermediate 283 and acetohydrazide |
| 402 | (S)-1-ethyl-3-(5′-(2-(2-hydroxypropanoyl)hydra- zinecarbonyl)-4-(4-(trifluoromethyl)thiazol-2-yl)- 3,3′-bipyridin-6-yl)urea 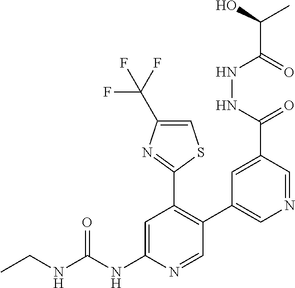 | LC/MS (ES⁺) [(M + H)⁺]: 524 for C₂₁H₂₀F₃N₇O₄S. ¹H NMR (300 MHz, d₆-DMSO): 1.11 (t, 3H), 1.30 (d, 3H), 3.21 (m, 2H), 4.15 (m, 1H), 5.56 (d, 1H), 7.55 (t, 1H), 8.21 (t, 1H), 8.26 (s, 1H), 8.37 (s, 1H), 8.56 (s, 1H), 8.64 (d, 1H), 9.06 (d, 1H), 9.49 (s, 1H), 9.81 (s, 1H), 10.53 (s, 1H). | Intermediate 9 and (S)-2- hydroxypropanoic acid |

### Intermediate 403

### (S)-1-ethyl-3-(5′-(2-(2-(triethylsilyloxy)propanoyl)hydrazinecarbonyl)-4-(4-(trifluoromethyl)thiazol-2-yl)-3,3′-bipyridin-6-yl)urea

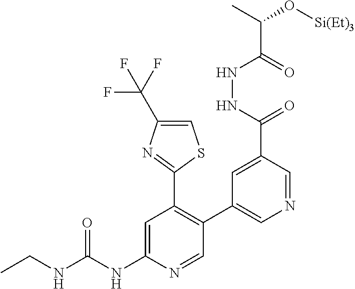

(S)-1-ethyl-3-(5′-(2-(2-hydroxypropanoyl)hydrazinecarbonyl)-4-(4-(trifluoromethyl)thiazol-2-yl)-3,3′-bipyridin-6-yl)urea (Intermediate 402, 260 mg, 0.50 mmol) was suspended in a CH₂Cl₂ (10 mL) solution containing 2,6-lutidine (213 mg, 1.99 mmol). The suspension was cooled to 0° C. (ice-water bath). Triethylsilyl trifluoromethanesulfonate (0.337 mL, 1.49 mmol) was added in a single portion via a micro syringe. The reaction mixture was slowly warmed to room temperature where it was allowed to react for 5 h. The reaction mixture became homogeneous, and analysis showed complete conversion to the silyl protected compound. The reaction mixture was diluted with CH₂Cl₂, washed with NaHCO₃ (sat.) and brine, dried organic over Na₂SO₄, filtered and concentrate by rotary evaporation. The crude reaction mixture was purified by silica gel flash column chromatography (95:5 CH₂Cl₂ MeOH) to give 205 mg of the title compound.

LC/MS (ES⁺)[(M+H)⁺]: 638 for C₂₇H₃₄F₃N₇O₄SSi.

### Intermediate 404

### (S)-tert-butyl cyclohexyl(5-(6′-(3-ethylureido)-4′-(4-(trifluoromethyl)thiazol-2-yl)-3,3′-bipyridin-5-yl)-1,3,4-oxadiazol-2-yl)methylcarbamate

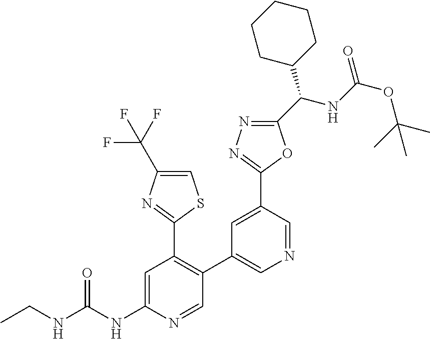

In a glass vial, (S)-tert-butyl 1-cyclohexyl-2-(2-(6′-(3-ethylureido)-4′-(4-(trifluoromethyl)thiazol-2-yl)-3,3′-bipyridine-5-carbonyl)hydrazinyl)-2-oxoethylcarbamate (Intermediate 392, 459 mg, 0.66 mmol) was dissolved in a ACN solution containing carbon tetrachloride (0.321 mL, 3.32 mmol) and DBU (1,8-Diazabicyclo[5.4.0]-undec-7-ene) (0.497 mL, 3.32 mmol). Triphenyl phosphine (349 mg, 1.33 mmol) was added in a single portion, and the reaction mixture was stirred at room temperature overnight. The reaction mixture was diluted with EtOAc, washed with water/brine, dried over Na₂SO₄, filtered and concentrated to dryness by rotary evaporation. The concentrate was purified by silica gel flash column chromatography (95:5 CH₂Cl₂/MeOH).

LC/MS (ES⁺)[(M+H)⁺]: 673 for C₃₁H₃₅F₃N₈O₄S.

¹H NMR (300 MHz, d₆-DMSO): 0.95-1.45 (m, 6H), 1.12 (t, 3H), 1.37 (s, 9H), 1.62-1.89 (m, 5H), 3.22 (m, 2H), 4.71 (m, 1H), 7.55 (t, 1H), 7.66 (d, 1H), 8.25 (s, 1H), 8.27 (s, 1H), 8.42 (s, 1H), 8.56 (s, 1H), 8.73 (s, 1H), 9.18 (s, 1H), 9.51 (s, 1H).

### Intermediates 405-410

The following Intermediates were prepared according to the procedure described for Intermediate 404 using the starting materials indicated in the table.

| Int | Compound | Data | SM |
|---|---|---|---|
| 405 | (S)-tert-butyl 3-(5-(6′-(3-ethylureido)-4′-(4-(trifluoromethyl)thiazol- 2-yl)-3,3′-bipyridin-5-yl)-1,3,4-oxadiazol-2-yl)morpholine-4- carboxylate 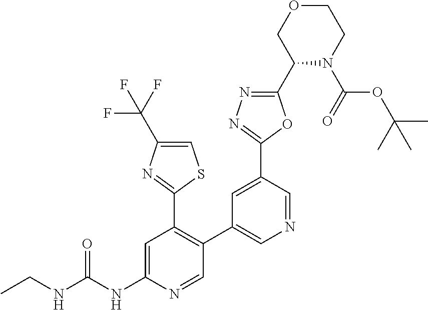 | LC/MS (ES⁺) [(M + H)⁺]: 647 for C₂₈H₂₉F₃N₈O₅S. | Intermediate 393 |
| 406 | (R)-tert-butyl 3-(2-(6′-(3-ethylureido)-4′-(4-(trfluoromethyl)thiazol- 2-yl)-3,3′-bipyridine-5-carbonyl)hydrazinecarbonyl)morpholine-4- carboxylate 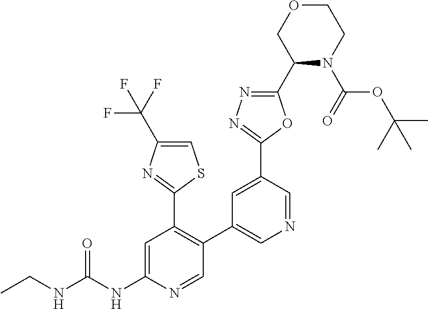 | LC/MS (ES⁺) [(M + H)⁺]: 647 for C₂₈H₂₉F₃N₈O₅S. | Intermediate 394 |
| 407 | (S)-tert-butyl cyclohexyl(5-(6-(3-ethylureido)-4-(4-(trifluoromethyl) thiazol-2-yl)-3,4′-bipyridin-2′-yl)-1,3,4-oxadiazol-2-yl)methyl- carbamate 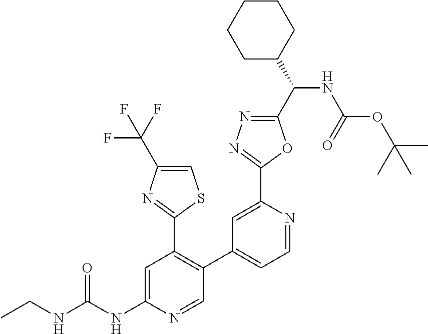 | LC/MS (ES⁺) [(M + H)⁺]: 673 for C₃₁H₃₅F₃N₈O₄S. | Intermediate 395 |
| 408 | (S)-tert-butyl 3-(5-(6-(3-ethylureido)-4-(4-(trifluoromethyl)thiazol- 2-yl)-3,4′-bipyridin-2′-yl)-1,3,4-oxadiazol-2-yl)morpholine-4- carboxylate 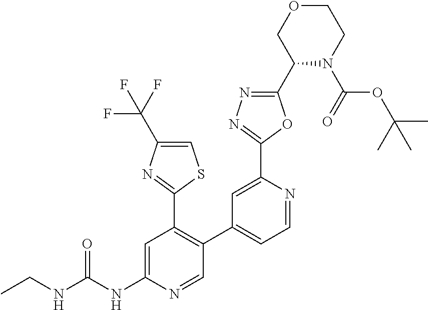 | LC/MS (ES⁺) [(M + H)⁺]: 647 for C₂₉H₃₃F₃N₈O₄S. | Intermediate 396 |
| 409 | (R)-tert-butyl 3-(5-(6-(3-ethylureido)-4-(4-(trifluoromethyl)thiazol- 2-yl)-3,4′-bipyridin-2′-yl)-1,3,4-oxadiazol-2-yl)morpholine-4- carboxylate 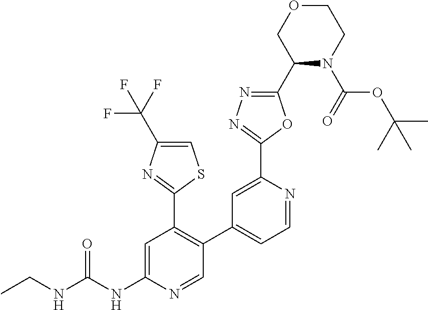 | LC/MS (ES⁺) [(M + H)⁺]: 647 for C₂₉H₃₃F₃N₈O₄S. | Intermediate 397 |
| 410 | (S)-tert-butyl 1-(5-(6-(3-ethylureido)-4-(4-(trifluoromethyl)thiazol- 2-yl)-3,4′-bipyridin-2′-yl)-1,3,4-oxadiazol-2-yl)-2-methylpropyl (methyl)carbamate 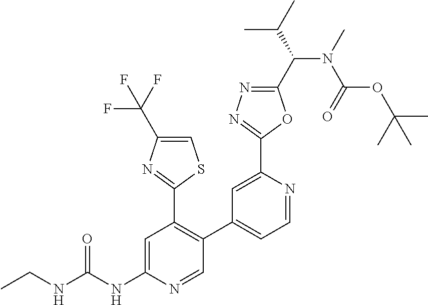 | LC/MS (ES⁺) [(M + H)⁺]: 647 for C₂₉H₃₃F₃N₈O₄S. ¹H NMR (300 MHz, d₆-DMSO): 0.95 (m, 6H), 1.11 (t, 3H), 1.40 (s, 9H), 2.77 (s, 3H), 3.22 (m, 2H), 4.90 (m, 1H), 5.18 (m, 1H), 7.52 (t, 1H), 7.59 (dd, 1H), 8.01 (s, 1H), 8.18 (s, 1H), 8.43 (s, 1H), 8.61 (s, 1H), 8.77 (d, 1H), 9.55 (s, 1H). | Intermediate 398 |

### Intermediate 411 and Intermediate 412

### 7-bromo-2-(2-hydroxyethyl)-2,3-dihydrophthalazine-1,4-dione and 6-bromo-2-(2-hydroxyethyl)-2,3-dihydrophthalazine-1,4-dione

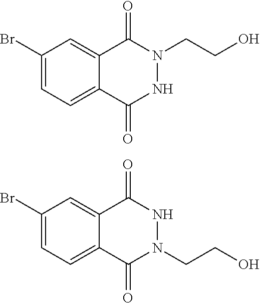

In a microwave vessel, 5-bromoisobenzofuran-1,3-dione (500 mg, 2.20 mmol) was suspended in an ethanolic solution containing 2-hydrazinylethanol (0.332 mL, 4.41 mmol). The vial was sealed and heated to reflux. The reaction mixture became homogeneous upon heating. After for 12 hours, the reaction was cooled to room temperature. Solids precipitated from solution and were collected by filtration, washed with ethanol, and dried in vacuo. Analysis showed the ratio of desired products to be 1:1 with about 30% of an unidentified side product. Isolation gave 340 mg of a 1:1 mixture of the title compounds which were not further purified.

LC/MS (ES⁺)[(M+H)⁺]: 285, 287 for C₁₀H₉BrN₂O₃.

### Intermediate 413

### tert-butyl 6′-(3-ethylureido)-2-((1R,3r,5S)-8-methyl-8-azabicyclo[3.2.1]octan-3-yloxy)-4′-(4-(trifluoromethyl)thiazol-2-yl)-3,3′-bipyridine-5-carboxylate

The title compound was prepared as described for Intermediate 2 from Intermediate 12 and Intermediate 349.

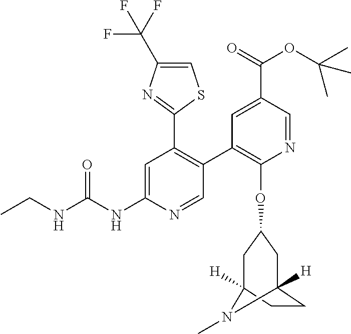

LC/MS (ES⁺)[(M+H)⁺]: 633 for C₃₀H₃₅F₃N₆O₄S.

### Intermediate 414

### 1-ethyl-3-(5′-(hydrazinecarbonyl)-2′-((1R,3r,5S)-8-methyl-8-azabicyclo[3.2.1]octan-3-yloxy)-4-(4-(trifluoromethyl)thiazol-2-yl)-3,3′-bipyridin-6-yl)urea

The title compound was prepared as described for Intermediate 9 from Intermediate 413 and hydrazine hydrate.

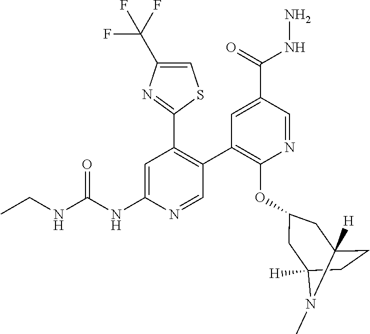

LC/MS (ES⁺)[(M+H)⁺]: 591 for C₂₆H₂₉F₃N₈O₃S

### Intermediate 415

### 6-(3-ethylureido)-4-(4-(6-methoxypyridin-2-yl)thiazol-2-yl)pyridin-3-ylboronic acid

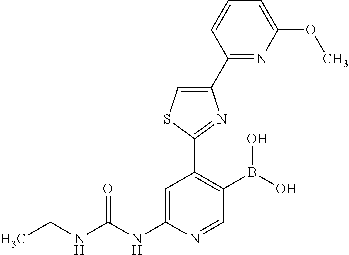

DMSO (36 mL) was added to a dry suspension of 1-(5-bromo-4-(4-(6-methoxypyridin-2-yl)thiazol-2-yl)pyridin-2-yl)-3-ethylurea (Intermediate 293, 2.5 g, 5.76 mmol), PdCl₂(dppf)-CH₂Cl₂ adduct (430 mg, 0.53 mmol), 4,4,4′,4′,5,5,5′,5′-octamethyl-2,2′-bi(1,3,2-dioxaborolane) (3 g, 11.81 mmol), and potassium acetate (1 g, 10.19 mmol) under vacuum. The resulting suspension was warmed to 80° C. and treated with triethyl amine (1 ml, 7.17 mmol) and stirred under nitrogen at this temperature for 16 hours. The reaction was diluted with water (100 ml), and held at 100° C. for 1 hr, cooled to room temperature, and filtered to give crude product as a peach filter cake. This material was suspended in ethyl acetate (200 ml), warmed to 70° C. for 1 hour, and filtered hot to give (1.44 g, 62.7%) of a peach solid as a 3:1 mixture of title boronic acid and the reduced material.

MS (EI) (M+H)⁺ 400 for C₁₇H₁₉BN₅O₄S (M−H)⁻ 398 for C₁₇H₁₇BN₅O₄S

¹H NMR (DMSO-d6) δ: 8.41 (t, J=5.46 Hz, 1H), 8.25 (s, 1H), 7.96 (s, 1H), 7.86 (s, 1H), 7.77 (d, J=7.16 Hz, 1H), 6.84 (d, J=7.72 Hz, 1H), 3.97 (s, 3H), 3.21 (d, J=7.35 Hz, 2H), 1.08-1.14 (m, 3H).

### Intermediate 416

### 1-ethyl-3-(2′-(hydrazinecarbonyl)-4-(4-(6-methoxypyridin-2-yl)thiazol-2-yl)-3,4′-bipyridin-6-yl)urea

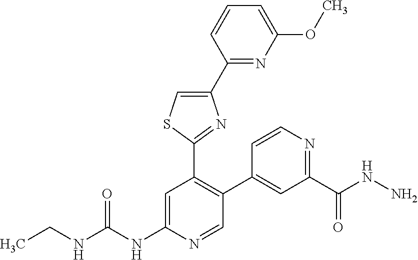

Intermediate 416 was synthesized according to the procedure described for intermediate 22 from Intermediate 417 and hydrazine.

MS (EI) (M+H)⁺ 491 for C₂₃H₂₃N₈O₃S (M−H)⁻ 489 for C₂₃H₂₁N₈O₃S;

### Intermediate 417

### methyl 6-(3-ethylureido)-4-(4-(6-methoxypyridin-2-yl)thiazol-2-yl)-3,4′-bipyridine-2′-carboxylate

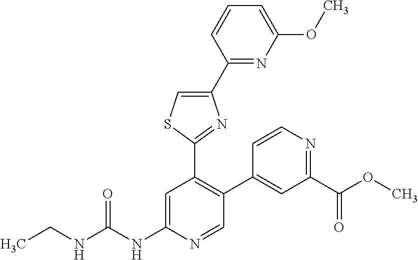

A mixture of 6-(3-ethylureido)-4-(4-(6-methoxypyridin-2-yl)thiazol-2-yl)pyridin-3-ylboronic acid (Intermediate 415, 400 mg, 1.00 mmol), methyl 4-bromopicolinate (216 mg, 1.00 mmol), dicyclohexyl(2′,4′,6′-triisopropylbiphenyl-2-yl)phosphine (143 mg, 0.30 mmol), Pd₂ dba₃ (45.9 mg, 0.05 mmol) and Cs₂CO₃ (392 mg, 1.20 mmol), under vacuum was treated with 1,4-dioxane (20 mL) and water (5 mL). The reaction mixture was placed in oil bath at 80° C., and held at that temperature for 2 hours, The reaction was cooled to room temperature, diluted with ethyl acetate (100 ml), water (50 ml), and brine (5 ml), and the layers were separated. The aqueous phase was extracted with ethyl acetate (3×50 ml), and the combined organic layers were washed with brine, dried over sodium sulfate, filtered, and the solvents were removed under reduced pressure. The resulting residue was purified by chromatography on silica gel eluting with a gradient of methanol in methylene chloride. The major peak was concentrated, and precipitated by the addition of acetonitrile to give the title compound as a tan solid (480 mg, 98%)

MS (EI) (M+H)⁺ 491 for C₂₃H₂₃N₆O₄S (M−H) 489 for C₂₃H₂₁N₆O₄S

¹H NMR (DMSO-d6) δ: 9.52 (s, 1H), 8.72 (d, J=4.90 Hz, 1H), 8.35-8.37 (m, 1H), 8.33 (br. s., 1H), 8.21 (s, 1H), 8.01 (s, 1H), 7.71 (t, J=7.82 Hz, 1H), 7.63 (d, J=5.09 Hz, 1H), 7.58 (t, J=5.18 Hz, 1H), 7.16 (d, J=7.35 Hz, 1H), 6.78 (d, J=8.10 Hz, 1H), 3.91 (s, 3H), 3.84 (s, 3 H), 3.22 (tt, J=7.16, 6.40 Hz, 2H), 1.11 (t, J=7.06 Hz, 3H).

### Intermediate 418

### 2-(5-bromopyridin-3-yl)-5-methyl-1,3,4-oxadiazole

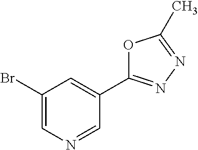

A suspension of 5-bromonicotinohydrazide (Intermediate 433, 2.3 g, 10.65 mmol) in 1,1,1-trimethoxyethane (20 ml, 166.46 mmol) was heated reflux and treated with concentrated aqueous HCl (drop), the resulting clear colorless solution was refluxed for 20 minutes, treated with DBU (0.2 ml, 1.33 mmol) and refluxed an additional 20 min. The material was concentrated under reduced pressure to give a tan gum which was purified by flash chromatography on silica gel eluting with a gradient of ethyl acetate in hexanes to give the title compound as a white solid (2.47 g, 97%)

MS (EI) (M+H)⁺ 240/242 for C₈H₇BrN₃O

¹H NMR (DMSO-d6) δ: 9.10 (d, J=1.51 Hz, 1H), 8.93 (d, J=2.07 Hz, 1H), 8.52 (t, J=1.60 Hz, 1H), 2.61 (s, 3H);

¹³C NMR (DMSO-d6) δ: 164.75 (s, 1 C), 160.98 (s, 1 C), 152.90 (s, 1 C), 145.43 (s, 1 C), 135.97 (s, 1 C), 121.60 (s, 1 C), 120.58 (s, 1 C), 10.61 (s, 1 C).

### Intermediate 419

### 1-ethyl-3-(5-(5-(hydrazinecarbonyl)-4-(1-methyl-1H-1,2,4-triazol-5-yl)thiazol-2-yl)-4-(4-(6-methoxypyridin-2-yl)thiazol-2-yl)pyridin-2-yl)urea

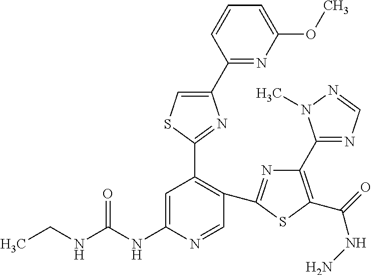

A solution of crude methyl 2-(6-(3-ethylureido)-4-(4-(6-methoxypyridin-2-yl)thiazol-2-yl)pyridin-3-yl)-4-(1-methyl-1H-1,2,4-triazol-5-yl)thiazole-5-carboxylate (Intermediate 420, 250 mg, 0.43 mmol) in ethanol was treated with hydrazine (0.5 mL, 0.43 mmol), and the pale grey solution was heated to reflux for 16 hours. The resulting pale grey suspension was filtered to give the title compound as a grey solid (200 mg, 0.35 mmol, 80%).

MS (EI) (M+H)⁺ 578 for C₂₄H₂₄N₁₁O₃S₂) (M−H)⁻ 576 for C₂₄H₂₂N₁₁O₃S₂

¹H NMR (DMSO-d6) δ: 11.81 (br. s., 1H), 9.68 (s, 1H), 8.82 (s, 1H), 8.47 (s, 1H), 8.24 (s, 1H), 8.12 (s, 1H), 7.74 (t, J=7.72 Hz, 1H), 7.56 (br. s., 1H), 7.42 (d, J=7.35 Hz, 1H), 6.81 (d, J=8.29 Hz, 1H), 3.95 (d, J=3.01 Hz, 6H), 3.12-3.26 (m, 2H), 1.09-1.16 (m, 3H).

### Intermediate 420

### methyl 2-(6-(3-ethylureido)-4-(4-(6-methoxypyridin-2-yl)thiazol-2-yl)pyridin-3-yl)-4-(1-methyl-1H-1,2,4-triazol-5-yl)thiazole-5-carboxylate

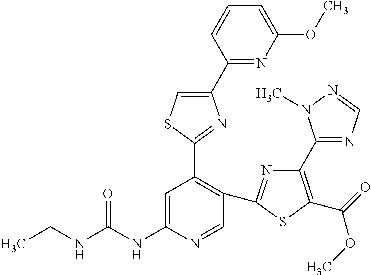

1,4-Dioxane (20 mL) and water (5 mL) were added to a mixture of 6-(3-ethylureido)-4-(4-(6-methoxypyridin-2-yl)thiazol-2-yl)pyridin-3-ylboronic acid (Intermediate 415, 400 mg, 1.00 mmol), methyl 2-chloro-4-(1-methyl-1H-1,2,4-triazol-5-yl)thiazole-5-carboxylate (Intermediate 44, 259 mg, 1.00 mmol), Pd₂ dba₃ (45.9 mg, 0.05 mmol), dicyclohexyl(2′,4′,6′-triisopropylbiphenyl-2-yl)phosphine (143 mg, 0.30 mmol), and Cs₂CO₃ (392 mg, 1.20 mmol), under vacuum. The suspension was placed in oil bath at 80° C., purged with nitrogen, and heated for 30 minutes. When the reaction was complete by LCMS, it was cooled to room temperature, diluted with water (100 ml), and extracted with ethyl acetate (4×75 ml). The combined organic layers were washed with brine, dried over sodium sulfate, filtered, and concentrated under reduced pressured, and the residue was purified by silica gel chromatography eluting with a gradient of methanol in methylene chloride to give the title compound as a beige solid (265 mg, 0.46 mmol, 45.8%).

MS (EI) (M+H)⁺ 578 for C₂₅H₂₃N₉O₄S₂ (M−H)⁻ 576 for C₂₅H₂₁N₉O₄S₂

¹H NMR (DMSO-d6) δ: 9.71 (s, 1H), 8.78 (s, 1H), 8.48 (s, 1H), 8.17 (s, 1H), 8.06 (s, 1H), 7.75 (t, J=7.82 Hz, 1H), 7.49 (br. s., 1H), 7.43 (d, J=7.35 Hz, 1H), 6.81 (d, J=8.10 Hz, 1H), 3.95 (s, 3H), 3.74 (s, 3H), 3.65 (s, 3H), 3.10-3.28 (m, 2H), 1.11 (t, J=7.16 Hz, 3H);

### Intermediate 421

### 1-ethyl-3-(5′-(hydrazinecarbonyl)-4-(4-(pyridin-4-ylmethyl)thiazol-2-yl)-3,3′-bipyridin-6-yl)urea

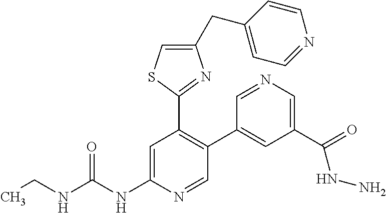

Intermediate 421 was synthesized according to the procedure described for Intermediate 22 from Intermediate 422 and hydrazine.

MS (EI) (M+H)⁺475 for C₂₃H₂₃N₈O₂S (M−H)⁻ 473 for C₂₃H₂₁N₈O₂S;

¹H NMR (DMSO-d6) δ: 9.98 (s, 1H), 9.45 (s, 1H), 8.97 (d, J=1.88 Hz, 1H), 8.52 (d, J=1.88 Hz, 1H), 8.41 (d, J=5.84 Hz, 2H), 8.28 (s, 1H), 8.09 (s, 2H), 7.66 (t, J=4.99 Hz, 1H), 7.53 (s, 1H), 7.08 (d, J=5.65 Hz, 2H), 4.59 (br. s., 2H), 4.03 (s, 2H), 3.20 (quin, J=6.97 Hz, 2H), 1.10 (t, J=7.16 Hz, 3H)

### Intermediate 422

### ethyl 6′-(3-ethylureido)-4′-(4-(pyridin-4-ylmethyl)thiazol-2-yl)-3,3′-bipyridine-5-carboxylate

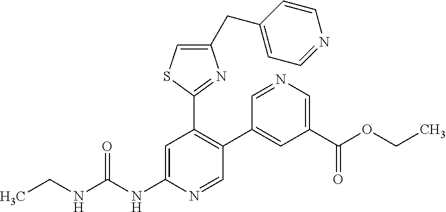

Intermediate 422 was synthesized as described for Intermediate 20 from Intermediate 423 and ethyl 5-(4,4,5,5-tetramethyl-1,3,2-dioxaborolan-2-yl)nicotinate.

MS (EI) (M+H)⁺489 for C₂₅H₂₅N₆O₃S (M−H) 487 for C₂₅H₂₃N₆O₃S;

¹H NMR (DMSO-d6) δ: 9.46 (s, 1H), 9.02 (d, J=2.07 Hz, 1H), 8.67 (d, J=2.26 Hz, 1H), 8.38 (d, J=5.46 Hz, 2H), 8.29 (s, 1H), 8.01-8.12 (m, 2H), 7.66 (t, J=5.27 Hz, 1H), 7.57 (s, 1H), 7.03 (d, J=5.65 Hz, 2H), 4.32 (q, J=6.78 Hz, 2H), 3.99 (s, 2H), 3.18-3.25 (m, 2 H), 1.31 (t, J=7.06 Hz, 3H), 1.10 (t, J=7.16 Hz, 3H).

### Intermediate 423

### 1-(5-bromo-4-(4-(pyridin-4-ylmethyl)thiazol-2-yl)pyridin-2-yl)-3-ethylurea

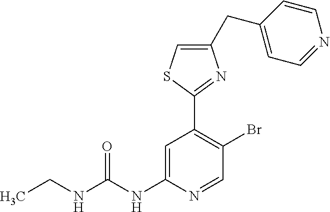

A solution of 1-bromo-3-(pyridin-4-yl)propan-2-one hydrobromide (Intermediate 424, 434 mg, 1.47 mmol) and 5-bromo-2-(3-ethylureido)pyridine-4-carbothioamide (Intermediate 5, 500 mg, 1.65 mmol) in ethanol (25 mL) was heated to reflux for 1 hour. The mixture was then cooled, diluted with water (100 ml), ethyl acetate (100 ml) and saturated aqueous sodium hydrogen carbonate, the layers were separated and the aqueous phase extracted with ethyl acetate (3×100 ml). The combined organic layers were washed with brine, dried over magnesium sulfate, filtered, concentrated under reduced pressure, and the resulting residue was purified by normal phase chromatography on silica gel, eluting with a gradient of ethyl acetate in hexanes to afford as a tan solid after trituration from dichloromethane with hexanes 364 mg (59%) of the title compound as a pale yellow powder.

MS (EI) (M+H)⁺ 418/420 for C₁₇H₁₇BrN₅OS (M−H)⁻ 416/418 for C₁₇H₁₅BrN₅OS;

¹H NMR (DMSO-d6) δ: 9.36 (s, 1H), 8.45-8.56 (m, 3H), 8.33 (s, 1H), 7.75 (s, 1H), 7.28-7.40 (m, 3H), 4.23 (s, 2H), 3.17 (quin, J=6.64 Hz, 2H), 1.08 (t, J=7.16 Hz, 3H);

### Intermediate 424

### 1-bromo-3-(pyridin-4-yl)propan-2-one

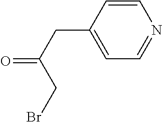

Bromine (0.65 ml, 12.5 mmol) was added to a solution of 1-(pyridin-4-yl)propan-2-one (770 mg, 5.70 mmol) in HBr (10 mL, 184.15 mmol, 33% in acetic acid). After 5 hours, the reaction was diluted with acetone (40 ml) and the resulting solution was stirred at room temperature for 19 hours. The resulting tan suspension was filtered to afford as a tan solid 755 mg of the title compound as a 2:1 mixture with 1,3-dibromo-1-(pyridin-4-yl)propan-2-one.

MS (EI) (M+H)⁺ 214/216 for C₈H₉BrNO

¹H NMR (DMSO-d6) δ: 8.82-8.95 (m, 2H), 8.65 (d, J=6.22 Hz, 1H), 8.05 (d, J=6.03 Hz, 1H), 7.92 (d, J=6.03 Hz, 2H), 5.89 (s, 1H), 4.57 (s, 2H), 4.38 (s, 1H), 4.30 (s, 2H)

### Intermediate 425

### ethyl 6′-(3-ethylureido)-6-(2-methoxyethoxy)-4′-(4-(trifluoromethyl)thiazol-2-yl)-3,3′-bipyridine-5-carboxylate

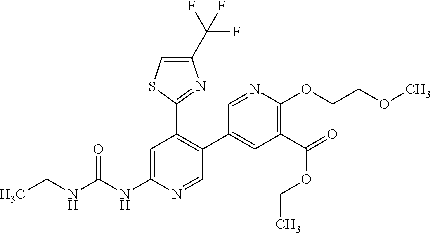

A mixture of ethyl 5-bromo-2-(2-methoxyethoxy)nicotinate (Intermediate 426, 500 mg, 1.64 mmol), 1-ethyl-3-(5-(4,4,5,5-tetramethyl-1,3,2-dioxaborolan-2-yl)-4-(4-(trifluoromethyl)thiazol-2-yl)pyridin-2-yl)urea (Intermediate 12, 500 mg, 1.13 mmol), Cs2CO3 (370 mg, 1.14 mmol), Pd₂dba₃ (27 mg, 0.03 mmol), and dicyclohexyl triisopropylbiphenylphosphine (170 mg, 0.36 mmol) in 1,4-dioxane (12 mL) was degassed, treated with water (3.00 mL), and then heated to 80° C. for 30 minutes. The reaction mixture was diluted with water (100 ml), brine (10 ml), and ethyl acetate (100 ml), and the layers were separated. The aqueous phase was extracted with ethyl acetate (3×50 ml), and the combined organics were washed with brine, dried over magnesium sulfate, filtered, concentrated under reduced pressure and purified by normal phase chromatography eluting with a gradient of ethyl acetate in hexanes to afford 90 mg of the title compound as a pale amber oil, which was used without further purification.

MS (EI) (M+H)⁺ 540 for C₂₃H₂₅F₃N₅O₅S (M−H) 538 for C₂₃H₂₃F₃N₅O₅S.

### Intermediate 426

### ethyl 5-bromo-2-(2-methoxyethoxy)nicotinate

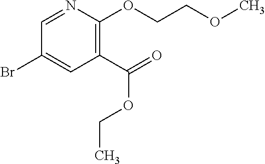

A solution of 5-bromo-2-(2-methoxyethoxy)nicotinic acid (Intermediate 427, 800 mg, 2.90 mmol) in ethanol (10 ml) was treated with sulfuric acid (drop), trimethoxymethane (10 ml) and refluxed for 1 hour. The resulting solution was cooled, diluted with water (100 ml), ethyl acetate (100 ml), and saturated bicarbonate (20 ml), and the layers were separated. The organic layers were washed with water and brine, then dried over magnesium sulfate, filtered, concentrated, and purified by normal phase chromatography on silica gel eluting with a gradient of ethyl acetate in hexanes to afford 500 mg of the title compound as a mixture of ethyl and methyl esters, as a colorless oil.

MS (EI) (M+H)⁺ 304/306 for C₁₁H₁₅BrNO₄ (M−H)⁻ 302/304 for C₁₁H₁₅BrNOₜ;

### Intermediate 427

### 5-bromo-2-(2-methoxyethoxy)nicotinic acid

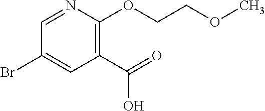

A solution of 2,5-dibromonicotinic acid (1 g, 3.5 mmol), 2-methoxyethanol (1.686 mL, 21.36 mmol) in DMF (10 mL) was treated with sodium hydride then warmed to 60° C. for 30 minutes. The reaction was diluted with water (100 ml), acidified (1N HCl), and extracted with ethyl acetate (3×100 ml). The combined organic layers were washed with brine, dried over magnesium sulfate, filtered, and the solvents were removed under reduced pressure. The resulting orange oil was purified by normal phase chromatography on silica gel eluting with a gradient of methanol in dichloromethane to give the title compound in solution with DMF, which was carried forward without further purification.

MS (EI) (M+H)⁺ 276/278 for C₉H₁₁BrNO₄ (M−H)⁻ 274/276 for C₉H₉BrNO₄

### Intermediate 428

### 3-bromo-5-(5-methyl-1,3,4-oxadiazol-2-yl)pyridine 1-oxide

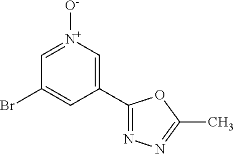

A solution of 2-(5-bromopyridin-3-yl)-5-methyl-1,3,4-oxadiazole (Intermediate 418, 870 mg, 3.62 mmol) in dichloromethane (25 mL) was treated with 3-chlorobenzoperoxoic acid (2031 mg, 9.06 mmol) and stirred at room temperate for 16 hours. Solvents were removed under reduced pressure and the residue was purified by normal phase chromatography on silica gel, eluting with a gradient of methanol in dichloromethane to give 900 mg of the title compound as a off white solid.

MS (EI) (M+H)⁺ 256/258 for C₈H₇BrN₃O₂;

### Intermediate 429

### 3-bromo-5-(5-(difluoromethyl)-4H-1,2,4-triazol-3-yl)pyridine

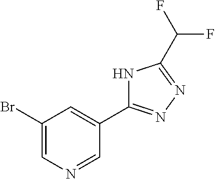

A mixture of 2-(5-bromopyridin-3-yl)-5-(difluoromethyl)-1,3,4-oxadiazole 2,2-difluoroacetate (Intermediate 430, 350 mg, 0.96 mmol) in ammonia (6 mL, 42.00 mmol, 7M in methanol) was heated to 130° C. for 15 min in a microwave reactor. The solvents were removed and the residue was purified by normal phase chromatography eluting with a gradient of ethyl acetate in hexanes to give 113 mg of the title compound as a white solid.

MS (EI) (M+H)⁺ 275/277 for C₈H₆BrF₂N₄ (M−H)⁻ 273/275 for C₈H₆BrF₂N₄;

¹H NMR (DMSO-d6) δ: 15.28 (br. s., 1H), 9.16 (d, J=1.51 Hz, 1H), 8.87 (d, J=2.07 Hz, 1H), 8.58 (s, 1H), 7.21 (d, J=53.31 Hz, 1H);

¹⁹F NMR (DMSO-d6) δ: −116.28 (br. s., 2 F);

### Intermediate 430

### 2-(5-bromopyridin-3-yl)-5-(difluoromethyl)-1,3,4-oxadiazole 2,2-difluoroacetate

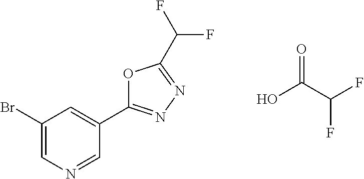

A suspension of 5-bromonicotinohydrazide (Intermediate 433, 2 g, 9.26 mmol) in toluene (10 mL) was treated with 2,2-difluoroacetic anhydride (1.611 g, 9.26 mmol) drop wise, the resulting suspension was heated to 70° C. for 30 minutes. The reaction was cooled to room temperature and let stir for 16 hours. The solvent was removed under reduced pressure and the residue was purified by normal phase chromatography on silica gel eluting with a gradient of ethyl acetate in hexanes to give the title compound as a white solid.

MS (EI) (M+H)⁺ 276/278 for C₈H₅BrF₂N₃O;

¹H NMR (DMSO-d6) δ: 14.22 (br. s., 1H), 9.19 (s, 1H), 9.00 (s, 1H), 8.63 (s, 1H), 7.58 (t, J=51.05 Hz, 1H), 6.28 (t, J=53.12 Hz, 1H);

¹⁹F NMR (DMSO-d6) δ: −120.83 (s, 2 F), −127.59 (s, 2 F);

### Intermediate 431

### 3-bromo-5-(5-(trifluoromethyl)-4H-1,2,4-triazol-3-yl)pyridine

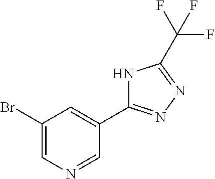

Example 431 was synthesized as described for Example 429 from Intermediate 432. The product was obtained as a white solid.

MS (EI) (M+H)⁺ 293/295 for C₈H₅BrF₃N₄ (M−H)⁻ 291/293 for C₈H₃BrF₃N₄;

¹H NMR (DMSO-d6) δ: 15.63 (br. s., 1H), 9.17 (d, J=1.51 Hz, 1H), 8.91 (d, J=2.07 Hz, 1H), 8.61 (t, J=1.98 Hz, 1H);

¹⁹H NMR (DMSO-d6) δ: −63.79 (br. s., 3 F);

### Intermediate 432

### 2-(5-bromopyridin-3-yl)-5-(trifluoromethyl)-1,3,4-oxadiazole

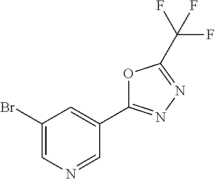

A mixture of trifluoroacetic anhydride (5 ml) and 5-bromonicotinohydrazide (Intermediate 433, 1 g, 3.24 mmol) was warmed to reflux for 5 minutes to afford an amber solution which was diluted with toluene (12 ml) and heated to reflux for an additional one hour. Solvents were removed under reduced pressure and the residue was purified by normal phase chromatography on silica gel, eluting with a gradient of dichloromethane in hexanes to afford 780 mg of the title compound as a white solid.

MS (EI) (M+H)⁺ 294/296 for C₈H₅BrF₃N₃O;

¹H NMR (DMSO-d6) δ: 9.24 (s, 1H), 9.05 (d, J=1.32 Hz, 1H), 8.70 (s, 1H);

¹⁹F NMR (DMSO-d6) δ: −64.21 (s, 3 F);

### Intermediate 433

### 5-bromonicotinohydrazide

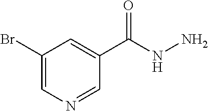

Hydrazine (0.8 g, 24 mmol) was added to a solution of methyl 5-bromonicotinate (5.15 g, 24 mol) in toluene (10 ml) and the mixture was heated to 80° C. for 16 hours. The reaction mixture was then diluted with ethyl acetate (30 ml), cooled to RT, filtered, and the white solid that was collected was washed with ethyl acetate to give 4.64 g of the title compound as off-white solid.

MS (EI) (M+H)⁺ 216/218 for C₆H₇BrN₃O (M−H)⁻ 214/216 for C₆H₅BrN₃O;

¹H NMR (DMSO-d6) δ: 10.00 (br. s., 1H), 8.94 (d, J=1.70 Hz, 1H), 8.82 (d, J=2.26 Hz, 1H), 8.31-8.40 (m, 1H), 4.63 (br. s., 2H);

¹³C NMR (DMSO-d6) δ: 162.72 (s, 1 C), 152.35 (s, 1 C), 146.63 (s, 1 C), 137.02 (s, 1 C), 130.46 (s, 1 C), 120.04 (s, 1 C);

### Intermediate 434

### 5-bromo-3-(5-methyl-1,3,4-oxadiazol-2-yl)pyridin-2-amine

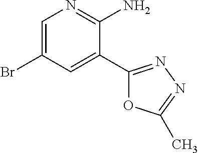

Carbontetrachloride (600 μl, 6.06 mmol) was added to a solution of N′-acetyl-2-amino-5-bromonicotinohydrazide (Intermediate 435, 270 mg, 0.99 mmol), triphenylphosphine (520 mg, 1.98 mmol), and DBU (300 μl, 1.99 mmol) in acetonitrile (50 mL). After stirring for 16 hours at RT the mixture was purified by normal phase chromatography on silica gel, eluting with a gradient of ethyl acetate in hexanes to afford 240 mg of the title compound as an off white solid.

MS (EI) (M+H)⁺ 255/257 for C₈H₈BrN₄O;

¹H NMR (DMSO-d6) δ: 8.27 (d, J=2.45 Hz, 1H), 8.08 (d, J=2.45 Hz, 1H), 7.45 (br. s., 2H), 2.58 (s, 3H);

### Intermediate 435

### N′-acetyl-2-amino-5-bromonicotinohydrazide

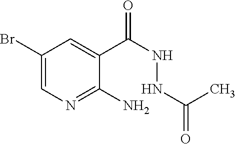

HATU (2.76 g, 7.26 mmol) was added to a solution of 2-amino-5-bromonicotinic acid (1.05 g, 4.84 mmol), acetohydrazide (0.466 g, 6.29 mmol), and DIEA (1.690 mL, 9.68 mmol) in DMF (20 mL) and the resulting solution was stirred at RT for 16 hours. The reaction was then diluted with water (250 ml) and let stir at RT for 60 hrs then filtered to afford 314 mg of the title compound as a white solid.

MS (EI) (M+H)⁺ 273/275 for C₈H₁₀BrN₄O₂ (M−H)⁻ 271/273 for C₈H₈BrN₄O₂;

¹H NMR (DMSO-d6) δ: 10.30 (s, 1H), 9.89 (s, 1H), 8.20 (d, J=2.26 Hz, 1H), 8.10 (d, J=2.07 Hz, 1H), 7.22 (s, 2H), 1.91 (s, 3H);

¹³C NMR (DMSO-d6) δ: 168.58 (s, 1 C), 165.53 (s, 1 C), 157.38 (s, 1 C), 151.98 (s, 1 C), 138.33 (s, 1 C), 109.02 (s, 1 C), 103.80 (s, 1 C), 20.47 (s, 1 C);

### Intermediate 436

### 4-bromo-2-(5-methyl-1,3,4-oxadiazol-2-yl)pyridine 1-oxide

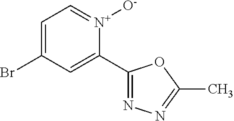

Intermediate 436 was synthesized according to the procedure for Intermediate 428 from Intermediate 176.

MS (EI) (M+H)⁺ 256/258 for C₈H₇BrN₃O₂;

### Intermediate 437

### 5-(6-amino-5-iodopyridin-3-yl)-1,3,4-oxadiazol-2(3H)-one

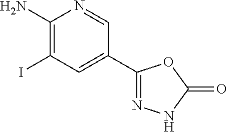

Hydrazine (2 ml) was added to a solution of (Z)-ethyl 5-iodo-6-(1-methoxyethylideneamino)nicotinate (Intermediate 437, 3 g, 8.62 mmol) in ethanol (50 mL) and warmed to 80° C. for 2 hours. Hydrochloric acid (1M in 1,4-dioxane, 1 ml) was added and heating continued for 2 hours. Additional hydrazine (2 ml) was added and heating continued for 16 hours. Solvents were removed and the crude mixture was dissolved in DMF (20 ml), treated with DIEA (3 ml), and 1,1′-carbonyl diimidazole (4 g). After stirring at RT for 8 hours, ethyl acetate (200 ml) was added and a solid was removed by filtration. The organic solution was washed with water (150 ml, then 50 ml) and brine (50 ml) then dried over magnesium sulfate. The solvents were removed under reduced pressure, and the residue was purified by normal phase chromatography on silica gel eluting with a gradient of methanol in methylene chloride. The major peak was precipitated from methylene chloride with hexanes to afford 200 mg (7.6%) of the title compound as a white powder.

MS (EI) (M+H)⁺ 305 for C₇H₆₁N₄O₂ (M−H)⁻ 303 for C₇H₄IN₄O₂;

¹H NMR (DMSO-d6) δ: 12.41 (br. s., 1H), 8.33 (d, J=2.07 Hz, 1H), 8.15 (d, J=2.07 Hz, 1H), 6.85 (d, J=0.94 Hz, 2H)

¹³C NMR (DMSO-d6) δ: 160.20 (s, 1 C), 154.27 (s, 1 C), 151.80 (s, 1 C), 145.55 (s, 1 C), 142.93 (s, 1 C), 10.28 (s, 1 C), 76.86 (s, 1 C)

### Intermediate 438

### (Z)-ethyl 5-iodo-6-(1-methoxyethylideneamino)nicotinate

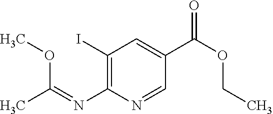

Hydrazine (2 ml) was added to a solution of ethyl 6-amino-5-iodonicotinate (Intermediate 439, 13 g, 31.16 mmol) in 2-methoxy ethanol (50 mL) and the mixture was heated to 135° C. for three hours. Solvents were removed and trimethylorthoacetate (10 ml), HCl (1 drop), and DBU (1 ml) were added and the mixture warmed to refluxed for 2 hours. The reaction was diluted with ethyl acetate (200 ml), washed sequentially with 100 ml each of water, saturated aqueous bicarbonate, and brine then dried over magnesium sulfate. The solvents were removed under reduced pressure and the resulting material was purified by normal phase chromatography on silica gel eluting with a gradient of ethyl acetate in hexanes to give 3.1 g of the title compound as an amber oil.

MS (EI) (M+H)⁺ 349 for C₁₁H₁₄IN₂O₃;

¹H NMR (DMSO-d6) δ: 8.80-8.83 (m, 1H), 8.56-8.60 (m, 1H), 4.32 (q, J=7.10 Hz, 2H), 3.83 (s, 3H), 1.87 (s, 3H), 1.32 (t, J=7.06 Hz, 3H)

### Intermediate 439

### ethyl 6-amino-5-iodonicotinate

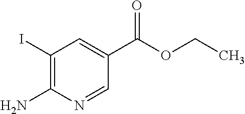

Ethyl 6-aminonicotinate (Intermediate 440, 8.7 g, 52.35 mmol) was suspended in ethanol (150 mL), and treated sequentially with Silver (I) sulfate (16.32 g, 52.35 mmol) and then diiodine (13.29 g, 52.35 mmol). The dark suspension was warmed to 80° C. for 5 hours then additional diiodine (1.4 g) and silver sulfate (1.7 g) were added. After 2 hours, the reaction mixture was cooled to rt, and a yellow solid was removed by filtration. The filtrate was concentrated under reduced pressure, and the resulting residue was purified by normal phase chromatography eluting with a gradient of ethyl acetate in hexanes. 2.8 grams of the title compound was precipitated from a mixture of hot ethyl acetate and hexanes as an off-white solid.

MS (EI) (M+H)⁺ 293 for C₈H₁₀IN₂O₂;

¹H NMR (DMSO-d6) δ: 10.31 (br. s., 2H), 8.51 (s, 1H), 8.45 (s, 1H), 4.26 (q, J=6.97 Hz, 2H), 1.28 (t, J=7.06 Hz, 3H)

¹³C NMR (DMSO-d6) δ: 162.90 (s, 1 C), 158.53 (s, 1 C), 148.93 (s, 1 C), 145.52 (s, 1 C), 115.80 (s, 1 C), 78.35 (s, 1 C), 60.83 (s, 1 C), 14.12 (s, 1 C).

### Intermediate 440

### ethyl 6-aminonicotinate

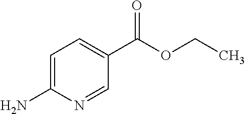

Thionyl chloride (15 ml) was added drop wise to a refluxing suspension of 6-aminonicotinic acid (10 g, 72.40 mmol) and sulfuric acid (0.5 mL, 9.38 mmol) in ethanol (300 mL, 72.40 mmol). The suspension was heated for an additional 16 hours at which time the solution was concentrated to dryness. The material was then suspended in ethyl acetate (250 ml) and washed with sodium hydroxide (6×50 ml, 1N), until the aqueous washes were basic, then with saturated aqueous bicarbonate and brine. The organic phase was dried over magnesium sulfate, filtered, and concentrated under reduced pressure to give 8.7 g of the title compound as a white solid.

MS (EI) (M+H)⁺ 167 for C₈H₁₁N₂O₂;

¹H NMR (DMSO-d6) δ: 8.49 (d, J=2.07 Hz, 1H), 7.81 (dd, J=8.67, 2.26 Hz, 1H), 6.83 (s, 2 H), 6.44 (d, J=8.67 Hz, 1H), 4.22 (q, J=7.16 Hz, 2H), 1.27 (t, J=7.16 Hz, 3H);

¹³C NMR (DMSO-d6) δ: 165.18 (s, 1 C), 162.48 (s, 1 C), 150.97 (s, 1 C), 137.49 (s, 1 C), 113.42 (s, 1 C), 107.01 (s, 1 C), 59.74 (s, 1 C), 14.24 (s, 1 C);

### Intermediate 441

### 1-(5-(5-(2-acetylhydrazinecarbonyl)-6-oxo-1,6-dihydropyridin-3-yl)-4-(4-(trifluoromethyl)thiazol-2-yl)pyridin-2-yl)-3-ethylurea

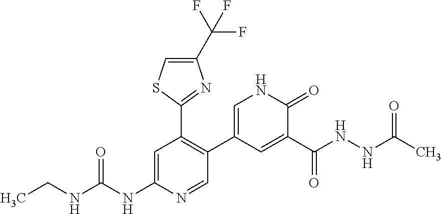

A solution of sodium nitrite (350 mg) in water (5 ml) was added dropwise to a suspension of 1-(6′-amino-5′-(5-methyl-1,3,4-oxadiazol-2-yl)-4-(4-(trifluoromethyl)thiazol-2-yl)-3,3′-bipyridin-6-yl)-3-ethylurea (Example 258, 40 mg, 0.08 mmol) and sulfuric acid (1 ml) in water (5.00 mL) at 0° C., the mixture was allowed to warm to RT over 16 hours. The reaction was diluted with saturated aqueous sodium bicarbonate (50 ml), extracted with ethyl acetate (3×50 ml), combined organics were washed with brine, dried over magnesium sulfate, filtered, and the solvents removed under reduced pressure. The residue was purified by normal phase chromatography on silica gel eluting with a gradient of methanol in dichloromethane to afford 25 mg of the title compound crude as a tan gum which was used without further purification.

MS (EI) (M+H)⁺ 510 for C₂₀H₁₉F₃N₇O₄S (M−H)⁻ 508 for C₂₀H₁₇F₃N₇O₄S;

### Intermediate 442

### 1-{5,5″-bis(hydrazinocarbonyl)-4′-[4-(trifluoromethyl)-1,3-thiazol-2-yl]-3,3′:5′,3″-terpyridin-2′-yl}-3-ethylurea

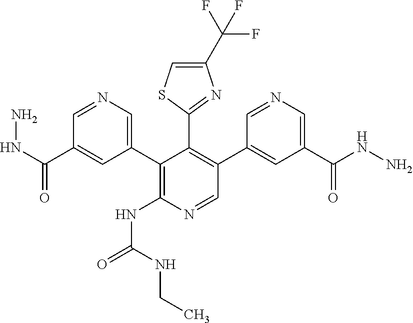

Acetyl chloride (0.4 ml) was added drop wise to a solution of di-tert-butyl 2,2′-({2′-[(ethylcarbamoyl)amino]-4′-[4-(trifluoromethyl)-1,3-thiazol-2-yl]-3,3′:5′,3″-terpyridine-5,5″-diyl}dicarbonyl)dihydrazine carboxylate (Intermediate 443, 38 mg, 0.05 mmol), and stirred at RT for 18 hours, solvent was removed and the material was used in the next step without purification.

MS (EI) (M+H)⁺ 587 for C₂₄H₂₂F₃N₁₀O₃S;

### Intermediate 443

### di-tert-butyl 2,2′-({2′-[(ethylcarbamoyl)amino]-4′-[4-(trifluoromethyl)-1,3-thiazol-2-yl]-3,3′:5′,3″-terpyridine-5,5″-diyl}dicarbonyl)dihydrazine carboxylate

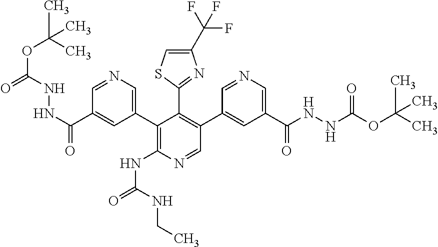

HATU (80 mg, 0.21 mmol) was added to a solution of 2′-[(ethylcarbamoyl)amino]-4′-[4-(trifluoromethyl)-1,3-thiazol-2-yl]-3,3′:5′,3″-terpyridine-5,5″-dicarboxylic acid (Intermediate 444, 60 mg, 0.11 mmol), tert-butyl hydrazinecarboxylate (50 mg, 0.38 mmol) and DIEA (0.2 mL, 1.15 mmol) in DMF (3 ml) and the resulting solution was stirred at RT for 20 hours. Ethyl acetate (50 ml) was added, followed by water (50 ml), and the layers were separated. The aqueous phase was extracted with ethyl acetate (3×50 m ml), and the combined organic layers were washed sequentially with saturated aqueous bicarbonate and brine, then dried over magnesium sulfate, filtered, and the solvent removed under reduced pressure. The material was purified by normal phase chromatography on silica gel eluting with a gradient of methanol in dichloromethane to afford 40 mg of the title compound as a tan solid.

MS (EI) (M+H)⁺ 787 for C₃₄H₃₈F₃N₁₀O₇S (M−H)⁻ 785 for C₃₄H₃₆F₃N₁₀O₇S;

¹H NMR (DMSO-d6) δ: 10.41 (d, J=0.75 Hz, 2H), 9.03 (d, J=8.48 Hz, 2H), 8.94 (s, 1H), 8.90 (s, 1H), 8.79 (s, 1H), 8.63 (s, 1H), 8.45 (d, J=5.84 Hz, 2H), 8.36 (d, J=0.94 Hz, 1H), 8.13 (d, J=11.30 Hz, 3H), 3.16 (d, J=5.27 Hz, 2H), 1.43 (s, 18H), 1.02-1.13 (m, 3H)

### Intermediate 444

### 2′-[(ethylcarbamoyl)amino]-4′-[4-(trifluoromethyl)-1,3-thiazol-2-yl]-3,3′:5′,3″-terpyridine-5,5″-dicarboxylic acid

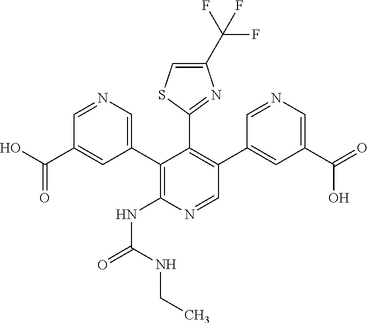

A degassed solution of 1-(3,5-dibromo-4-(4-(trifluoromethyl)thiazol-2-yl)pyridin-2-yl)-3-ethylurea (Intermediate 445, 60 mg, 0.13 mmol), ethyl 5-(4,4,5,5-tetramethyl-1,3,2-dioxaborolan-2-yl)nicotinate (77 mg, 0.28 mmol), diphenylphospinoferocenyl palladium dichloride (10.34 mg, 0.01 mmol), and potassium carbonate (26.2 mg, 0.19 mmol) in acetonitrile (3 ml) and water (3.00 ml) under nitrogen was heated in a microwave reactor for 1 hour at 100° C. Lithium hydroxide (0.3 ml, 2N in water) was added and the solution was heated to 100° C. in a microwave reactor. The reaction was then diluted with ethyl acetate (50 ml) and water (50 ml), and the layers were separated. The aqueous phase was washed with ethyl acetate (50 ml), filtered then acidified with 1N hydrochloric acid and extracted with ethyl acetate (3×50 ml). The combined organic layers were washed with brine, dried over magnesium sulfate, filtered, and the solvents removed under reduced pressure to give 50 mg of the title compound as a pale yellow gum, which was used in the next step with no further purification.

MS (EI) (M+H)⁺ 559 for C₂₄H₁₈F₃N₆O₅S (M−H)⁻ 557 for C₂₄H₁₆F₃N₆O₅S;

¹H NMR (DMSO-d6) δ: 13.47 (br. s., 2H), 8.95 (d, J=1.88 Hz, 3H), 8.66 (d, J=2.07 Hz, 1H), 8.64 (s, 1H), 8.53 (d, J=1.88 Hz, 1H), 8.36 (s, 1H), 8.28 (s, 1H), 8.06-8.13 (m, 1H), 7.96-8.03 (m, 1H), 3.12-3.25 (m, 2H), 1.02-1.11 (m, 3H)

¹⁹F NMR (DMSO-d6) δ: −62.73 (s, 3 F)

### Intermediate 445

### 1-(3,5-dibromo-4-(4-(trifluoromethyl)thiazol-2-yl)pyridin-2-yl)-3-ethylurea

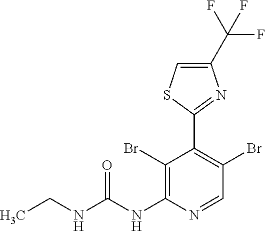

A mixture of 1-ethyl-3-(4-(4-(trifluoromethyl)thiazol-2-yl)pyridin-2-yl)urea (from synthesis of Intermediate 12, 350 mg, 1.11 mmol) and 1-bromopyrrolidine-2,5-dione (350 mg, 1.97 mmol) in DMF (30 mL) was heated to 70° C. for 2 hours. The reaction was diluted with water (300 ml) to afford a brown precipitate which was recovered by filtration. This solid was purified by normal phase chromatography on silica gel eluting with a gradient of ethyl acetate in hexanes to give 90 mg of the title compound as a brown solid.

MS (EI) (M+H)⁺ 475 for C₁₂H₁₀Br₂F₃N₄OS (M−H)⁻ 473 for C₁₂H₈Br₂F₃N₄OS;

¹H NMR (DMSO-d6) δ: 8.87 (s, 1H), 8.60 (s, 1H), 8.24 (br. s., 2H), 3.23 (qd, J=6.97, 6.03 Hz, 2H), 1.11 (t, J=7.16 Hz, 3H);

¹⁹H NMR (DMSO-d6) δ: −61.99 (s, 3 F);

### Intermediate 446

### 1-ethyl-3-(5-(6-(hydrazinecarbonyl)pyrazin-2-yl)-4-(4-(6-methoxypyridin-2-yl)thiazol-2-yl)pyridin-2-yl)urea

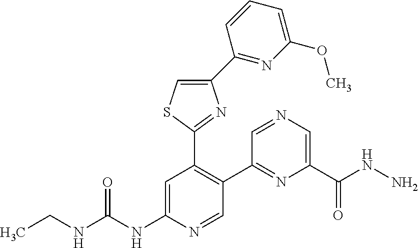

A solution of tert-butyl 2-(6-(6-(3-ethylureido)-4-(4-(6-methoxypyridin-2-yl)thiazol-2-yl)pyridin-3-yl)pyrazine-2-carbonyl)hydrazinecarboxylate (Intermediate 447, 30 mg, 0.05 mmol) in MeOH (20 mL), was treated with acetyl chloride (1 mL, 0.05 mmol) drop wise. After stirring at RT for 18 hours, solvents were removed under reduced pressure to give 26 mg of the title compound as a white solid, which was used without further purification.

MS (EI) (M+H)⁺ 492 for C₂₂H₂₂N₉O₃S (M−H) 490 for C₂₂H₂₀N₉O₃S;

### Intermediate 447

### tert-butyl 2-(6-(6-(3-ethylureido)-4-(4-(6-methoxypyridin-2-yl)thiazol-2-yl)pyridin-3-yl)pyrazine-2-carbonyl)hydrazinecarboxylate

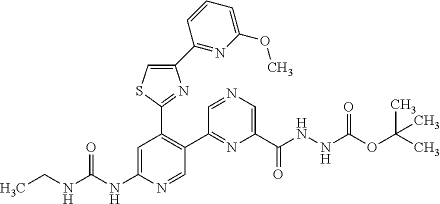

HATU (50 mg, 0.13 mmol) was added to a solution of 6-(6-(3-ethylureido)-4-(4-(6-methoxypyridin-2-yl)thiazol-2-yl)pyridin-3-yl)pyrazine-2-carboxylic acid (Intermediate 486, mg, 0.05 mmol), tert-butyl hydrazinecarboxylate (30 mg, 0.23 mmol), and DIEA (50 μL, 0.29 mmol) in DMF (6 mL), and the mixture stirred at RT for 48 hours. The reaction was diluted with ethyl acetate (50 ml) and water (50 ml), and the layers were separated. The aqueous phase was extracted with ethyl acetate (3×50 ml), and the combined organic layers were washed sequentially with saturated aqueous bicarbonate and brine then dried over magnesium sulfate, filtered, and the solvents were removed under reduced pressure. The resulting residue was purified by normal phase chromatography on silica gel, eluting with a gradient of ethyl acetate in hexanes to afford 30 mg of the title compound as a white solid.

MS (EI) (M+H)⁺ 592 for C₂₇H₃₀N₉O₅S (M−H)⁻ 590 for C₂₇H₂₈N₉O₅S;

¹H NMR (DMSO-d6) δ: 10.53 (s, 1H), 9.60 (s, 1H), 9.08 (s, 1H), 9.03 (s, 1H), 8.76 (s, 1H), 8.69 (s, 1H), 8.40 (s, 1H), 8.23 (s, 1H), 7.85 (br. s., 1H), 7.70 (s, 1H), 7.54 (br. s., 1H), 6.76 (d, J=8.29 Hz, 1H), 3.92 (s, 3H), 3.10-3.28 (m, 2H), 1.38 (s, 9H), 1.17 (t, J=7.16 Hz, 3H).

### Intermediate 448

### 1-(2-(6′-(3-ethylureido)-4′-(4-(trifluoromethyl)thiazol-2-yl)-3,3′-bipyridine-5-carbonyl)hydrazinyl)-2-methyl-1-oxopropan-2-yl acetate

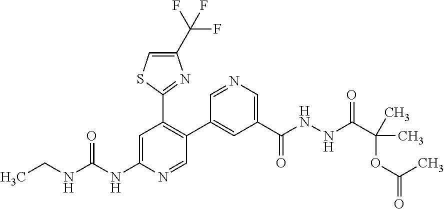

A solution of 1-ethyl-3-(5′-(hydrazinecarbonyl)-4-(4-(trifluoromethyl)thiazol-2-yl)-3,3′-bipyridin-6-yl)urea (Intermediate 9, 100 mg, 0.22 mmol) in pyridine (1.5 ml) was treated with 1-chloro-2-methyl-1-oxopropan-2-yl acetate (0.5 ml) and allowed to stir at RT for 30 minutes. Volatiles were removed under reduced pressure and the residue was purified by normal phase chromatography on silica gel eluting with ethyl acetate in hexanes to afford 100 mg of the title compound as an amber gum.

MS (EI) (M+H)⁺ 580 for C₂₄H₂₅F₃N₇O₅S (M−H) 578 for C₂₄H₂₃F₃N₇O₅S;

¹H NMR (DMSO-d6) δ: 10.60 (s, 1H), 9.95 (s, 1H), 9.50 (s, 1H), 9.06 (d, J=1.88 Hz, 1H), 8.65 (d, J=1.88 Hz, 1H), 8.57 (s, 1H), 8.37 (s, 1H), 8.25 (s, 1H), 8.22 (s, 1H), 7.55 (t, J=5.37 Hz, 1H), 3.12-3.27 (m, 2H), 2.03 (s, 3H), 1.56 (s, 6H), 1.11 (t, J=7.16 Hz, 3H); ¹⁹F NMR (DMSO-d6) δ: −62.41 (s, 3 F);

### Intermediates 449-463

The following Intermediates were prepared as described for Intermediate 448 using the starting materials indicated in the table.

| Int | Compound | Data | SM |
|---|---|---|---|
| 449 | 1-ethyl-3-(5′-(2-(3-oxobutanoyl)hydrazinecarbonyl)-4-(4-(trifluoro- methyl)thiazol-2-yl)-3,3′-bipyridin-6-yl)urea 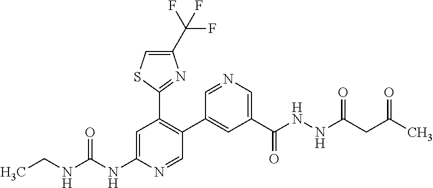 | MS (EI) (M + H)⁺ 536 for C₂₂H₂₁F₃N₇O₆S (M − H)⁻ 534 for C₂₂H₁₉F₃N₇O₆S; ¹H NMR (DMSO-d6) δ: 10.74 (s, 0 H), 10.26 (s, 1 H), 9.51 (s, 1 H), 9.05 (d, J = 1.88 Hz, 1 H), 8.65 (d, J = 1.88 Hz, 1 H), 8.57 (s, 1H), 8.38 (s, 1 H), 8.25 (s, 1 H), 8.22 (s, 1 H), 7.55 (br. s., 0 H), 3.45 (s, 2 H), 3.17-3.26 (m, 2 H), 2.22 (s, 3 H), 1.11 (t, J = 7.25 Hz, 3 H); ¹⁹F NMR (DMSO-d6) δ: −62.42 (s, 3 F) | Intermediate 9 and 4-methyleneoxetan- 2-one |
| 450 | 1-(5′-(2-(2-(benzyloxy)acetyl)hydrazinecarbonyl)-4-(4-(trifluoromethyl) thiazol-2-yl)-3,3′-bipyridin-6-yl)-3-ethylurea 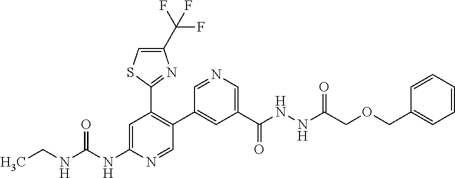 | MS (EI) (M + H)⁺ 600 for C₂₇H₂₅F₃N₇O₄S (M − H)⁻ 598 for C₂₇H₂₂F₃N₇O₄S; ¹H NMR (DMSO-d6) δ: 10.63 (br. s., 1 H), 10.08 (s, 1 H), 9.51 (s, 1 H), 9.06 (d, J = 1.88 Hz, 1 H), 8.66 (d, J = 2.07 Hz, 1 H), 8.57 (s, 1 H), 8.37 (s, 1 H), 8.25 (s, 1 H), 8.21 (t, J = 2.07 Hz, 1 H), 7.55 (t, J = 4.90 Hz, 1 H), 7.22-7.46 (m, 5 H), 4.61 (s, 2 H), 4.08 (s, 2 H), 3.21 (qd, J = 7.16, 5.84 Hz, 2 H), 1.11 (t, J = 7.16 Hz, 3 H); ¹⁹F NMR (DMSO-d6) δ: −62.41 (s, 3 F); | Intermediate 9 and 2-(benzyloxy)acetyl chloride |
| 451 | N,N-diethyl-2-(6′-(3-ethylureido)-4′-(4-(trifluoromethyl)thiazol-2-yl)- 3,3′-bipyridine-5-carbonyl)hydrazinecarboxamide 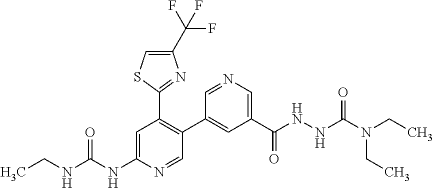 | MS (EI) (M + H)⁺ 551 for C₂₃H₂₆F₃N₈O₃S (M − H)⁻ 549 for C₂₃H₂₄F₃N₈O₃S; ¹H NMR (DMSO-d6) δ: 10.28 (s, 1 H), 9.50 (s, 1 H), 9.06 (s, 1 H), 8.64 (s, 1 H), 8.57 (s, 1 H), 8.43 (s, 1 H), 8.37 (s, 1 H), 8.25 (s, 1 H), 8.19 (s, 1 H), 7.56 (t, J = 5.65 Hz, 1 H), 3.12-3.30 (m, 6 H), 0.94-1.18 (m, 9 H); ¹⁹F NMR (DMSO-d6) δ: −62.40 (s, 3 F); | Intermediate 9 and diethylcarbamic chloride |
| 452 | 1-(5′-(2-(2-(dimethylamino)acetyl)hydrazinecarbonyl)-4-(4-(trifluoro- methyl)thiazol-2-yl)-3,3′-bipyridin-6-yl)-3-ethylurea 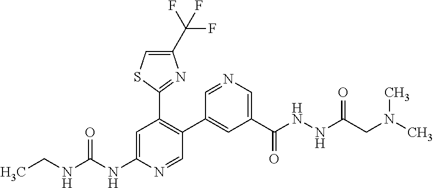 | MS (EI) (M + H)⁺ 537 for C₂₂H₂₄F₃N₈O₃S (M − H)⁻ 535 for C₂₂H₂₂F₃N₈O₃S; ¹H NMR (DMSO-d6) δ: 10.57 (br. s., 1 H), 9.88 (s, 1 H), 9.50 (s, 1 H), 9.05 (d, J = 2.07 Hz, 1 H), 8.65 (d, J = 1.88 Hz, 1 H), 8.57 (s, 1 H), 8.37 (s, 1 H), 8.25 (s, 1H), 8.21 (t, J = 1.98 Hz, 1 H), 7.92 (s, 1 H), 7.55 (t, J = 5.18 Hz, 1 H), 3.13-3.27 (m, J = 7.16, 7.16, 7.16, 6.03 Hz, 2 H), 3.02 (s, 2 H), 2.27 (s, 6 H), 1.11 (t, J = 7.16 Hz, 3 H); ¹⁹F NMR (DMSO-d6) δ: −62.41 (s, 3 F); | Intermediate 9 and 2-(dimethylamino) acetyl chloride |
| 453 | (9H-fluoren-9-yl)methyl 2-(2-(6′-(3-ethylureido)-4′-(4-(trifluoromethyl)thiazol-2-yl)-3,3′-bipyridine-5-carbonyl) hydrazinyl)-2-oxoethylcarbamate 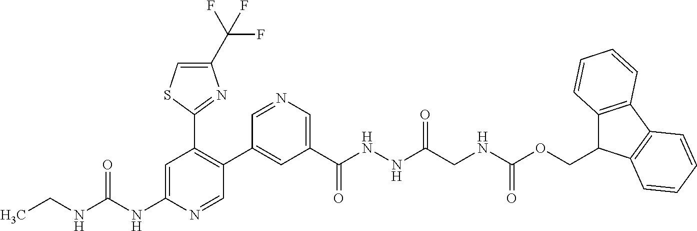 | MS (EI) (M + H)⁺ 731 for C₃₅H₃₀F₃N₈O₅S (M − H)⁻ 729 for C₃₅H₂₈F₃N₈O₅S; ¹H NMR (DMSO-d6) δ: 10.64 (br. s., 1 H), 10.13 (s, 1 H), 9.51 (s, 1 H), 9.05 (s, 1 H), 8.65 (s, 1 H), 8.56 (s, 1 H), 8.37 (s, 1 H), 8.24 (s, 1 H), 8.20 (br. s., 1 H), 7.89 (d, J = 7.35 Hz, 2 H), 7.72 (d, J = 7.35 Hz, 2 H), 7.68 (d, J = 6.22 Hz, 1 H), 7.55 (br. s., 1 H), 7.42 (t, J = 7.35 Hz, 2 H), 7.33 (t, 2 H), 4.12-4.42 (m, 3 H), 3.75 (d, J = 5.65 Hz, 2 H), 3.21 (quin, J = 6.64 Hz, 2 H), 1.10 (t, J = 7.16 Hz, 3 H); ¹⁹F NMR (DMSO-d6) δ: −62.39 (s, 3 F) | Intermediate 9 and (9H-fluoren-9- yl)methyl 2-chloro- 2-oxoethyl- carbamate |
| 454 | 1-ethyl-3-(5′-(2-(2-methoxyacetyl)hydrazinecarbonyl)-4-(4-(trifluoromethyl)thiazol- 2-yl)-3,3′-bipyridin-6-yl)urea 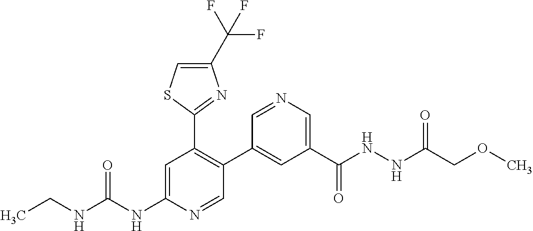 | MS (EI) (M + H)⁺ 524 for C₂₁H₂₁F₃N₇O₄S (M − H)⁻ 522 for C₂₁H₁₉F₃N₇O₄S; ¹H NMR (DMSO-d6) δ: 10.59 (s, 1 H), 10.02 (s, 1 H), 9.50 (s, 1 H), 9.06 (d, J = 1.88 Hz, 1 H), 8.65 (d, J = 1.88 Hz, 1 H), 8.57 (s, 1H), 8.37 (s, 1 H), 8.25 (s, 1 H), 8.21 (s, 1 H), 7.56 (br. s., 1 H), 3.98 (s, 2 H), 3.36 (s, 3 H), 3.14-3.27 (m, 2 H), 1.10 (t, J = 7.16 Hz, 3 H); ¹⁹F NMR (DMSO-d6) δ: −62.42 (s, 3 F); | Intermediate 9 and 2-methoxyacetyl chloride |
| 455 | ethyl 2-(6′-(3-ethylureido)-4′-(4-(trifluoromethyl)thiazol-2-yl)-3,3′-bipyridine-5-carbonyl) hydrazinecarboxylate 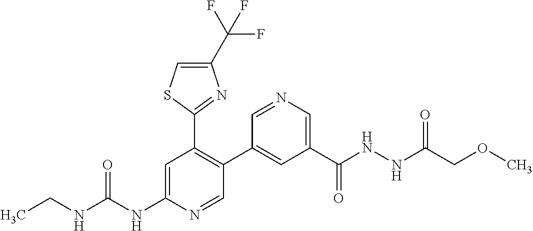 | MS (EI) (M + H)⁺ 524 for C₂₁H₂₁F₃N₇O₄S (M − H)⁻ 522 for C₂₁H₁₉F₃N₇O₄S; ¹H NMR (DMSO-d6) δ: 10.53 (s, 1 H), 9.51 (s, 1 H), 9.31 (br. s., 1 H), 9.04 (s, 1 H), 8.66 (s, 1 H), 8.57 (s, 1 H), 8.37 (s, 1 H), 8.24 (s, 1 H), 8.19 (br. s., 1 H), 7.55 (t, J = 5.56 Hz, 1 H), 4.08 (dt, J = 9.23, 6.97 Hz, 2 H), 3.21 (qd, J = 7.16, 6.03 Hz, 2 H), 1.16- 1.26 (m, 3 H), 1.11 (t, J = 7.16 Hz, 3 H); ¹⁹F NMR (DMSO-d6) δ: −62.43 (s, 3 F); | Intermediate 9 and ethyl carbono- chloridate |
| 456 | 1-ethyl-3-(5′-(2-formylhydrazinecarbonyl)-4-(4-(trifluoromethyl)thiazol- 2-yl)-3,3′-bipyridin-6-yl)urea 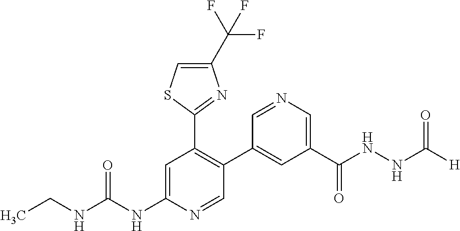 | MS (EI) (M + H)⁺ 480 for C₁₉H₁₇F₃N₇O₃S (M − H)⁻ 478 for C₁₉H₁₅F₃N₇O₃S; | Intermediate 9 and formic acid and HATU |
| 457 | 1-ethyl-3-(5′-(2-(1-hydroxycyclopropanecarbonyl)hydrazinecarbonyl)-4-(4- (trifluoromethyl)thiazol-2-yl)-3,3′-bipyridin-6-yl)urea 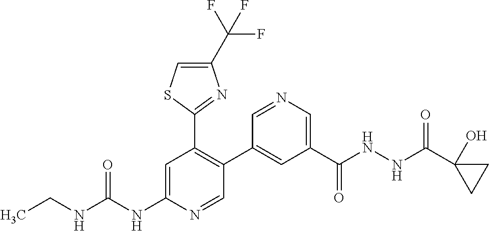 | MS (EI) (M + H)⁺ 536 for C₂₂H₂₁F₃N₇O₄S (M − H)⁻ 534 for C₂₂H₁₉F₃N₇O₄S; | Intermediate 9, 1- hydroxycyclopro- panecarboxylic acid and HATU |
| 458 | 2-(2-(6′-(3-ethylureido)-4′-(4-(trifluoromethyl)thiazol-2-yl)-3,3′-bipyridine-5-carbonyl) hydrazinyl)-N,N-dimethyl-2-oxoacetamide 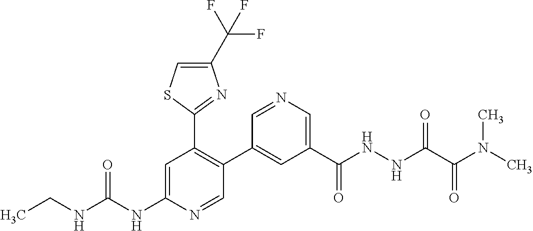 | MS (EI) (M + H)⁺ 551 for C₂₂H₂₂F₃N₈O₄S (M − H)⁻ 549 for C₂₂H₂₀F₃N₈O₄S; | Intermediate 9, 2- (dimethylamino)-2- oxoacetic acid and HATU |
| 459 | 1-ethyl-3-(5′-(2-(2-hydroxypropanoyl)hydrazinecarbonyl)-4-(4-(trifluoromethyl) thiazol-2-yl)-3,3′-bipyridin-6-yl)urea 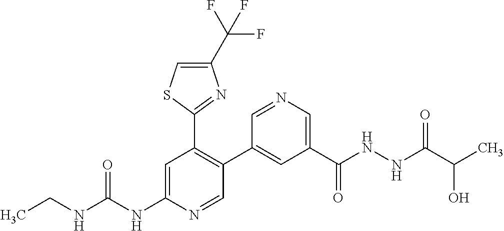 | MS (EI) (M + H)⁺ 524 for C₂₁H₂₁F₃N₇O₄S (M − H)⁻ 522 for C₂₁H₁₉F₃N₇O₄S; | Intermediate 9, 2- hydroxypropanoic acid and HATU |
| 460 | 2-(2-(6′-(3-ethylureido)-4′-(4-(trifluoromethyl)thiazol-2-yl)-3,3'-bipyridine-5-carbonyl)hydra- zinyl)-2-oxoethyl acetate 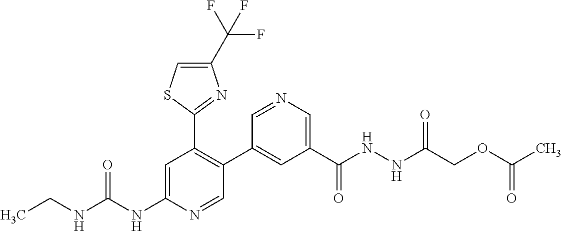 | MS (EI) (M + H)⁺ 552 for C₂₂H₂₁F₃N₇O₅S (M − H)⁻ 550 for C₂₂H₁₉F₃N₇O₅S; | Intermediate 9, 2- acetoxyacetic acid and HATU |
| 461 | (S)-tert-butyl 1-(2-(6′-(3-ethylureido)-4′-(4-(trifluoromethyl)thiazol-2-yl)-3,3′-bipyridine- 5-carbonyl)hydrazinyl)-3-methyl-1-oxobutan-2-ylcarbamate 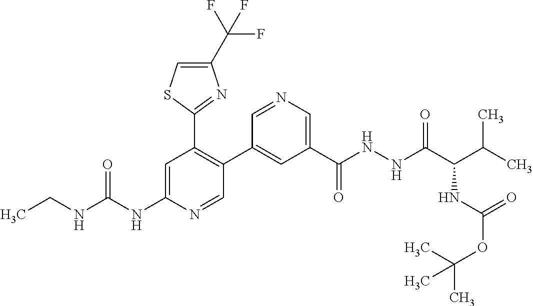 | MS (EI) (M + H)⁺ 651 for C₂₈H₃₄F₃N₈O₅S (M − H)⁻ 649 for C₂₈H₃₂F₃N₈O₅S; ¹H NMR (DMSO-d6) δ: 10.62 (s, 1 H), 10.11 (s, 1 H), 9.50 (s, 1 H), 9.06 (s, 1 H), 8.64 (s, 1 H), 8.57 (s, 1 H), 8.37 (s, 1 H), 8.25 (s, 1 H), 8.23 (br. s., 1 H), 7.55 (br. s., 1 H), 6.83 (d, J = 8.85 Hz, 1 H), 3.88 (t, J = 7.91 Hz, 1 H), 3.09-3.28 (m, 2 H), 1.84-2.07 (m, 1 H), 1.39 (s, 9 H), 1.10 (t, J = 7.16 Hz, 3 H), 0.97 (d, J = 6.59 Hz, 3 H), 0.90 (d, J = 6.59 Hz, 3 H); ¹⁹F NMR (DMSO-d6) δ: −62.42 (s, 3 F) | Intermediate 9 and (S)-tert-butyl 4- isopropyl-2,5- dioxooxazolidine-3- carboxylate |
| 462 | 1-ethyl-3-(5′-(2-(2-(2-methoxyethoxy)acetyl)hydrazinecarbonyl)-4-(4-(trifluoromethyl)thiazol-2-yl)- 3,3′-bipyridin-6-yl)urea 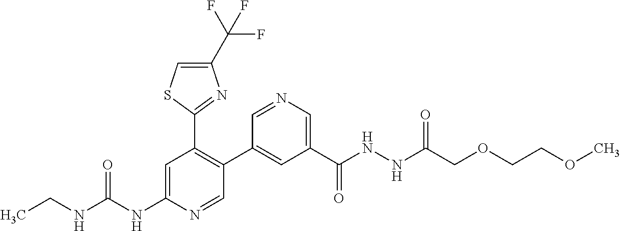 | MS (EI) (M + H)⁺ 568 for C₂₃H₂₅F₃N₇O₅S (M − H)⁻ 566 for C₂₃H₂₃F₃N₇O₅S; | Intermediate 9 and 2-(2-methoxyethoxy) acetyl chloride |
| 463 | 1-(5′-(2-(1-aminocyclopropanecarbonyl)hydrazinecarbonyl)-4-(5-methyl-4- (trifluoromethyl)thiazol-2-yl)-3,3′-bipyridin-6-yl)-3-ethylurea 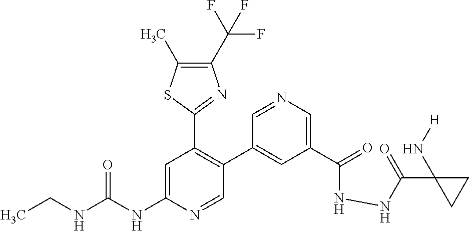 | MS (EI) (M + H)⁺ 549 for C₂₃H₂₄F₃N₈O₃S (M − H)⁻ 547 for C₂₃H₂₄F₃N₈O₃S; | Intermediate 237, 1- (tert-butoxycarbonyl- amino)cyclopropane- carboxylic acid and HATU, followed by HCl in methanol. |

### Intermediate 464

### 1-(5′-(2-(2-chloroacetyl)hydrazinecarbonyl)-4-(4-(trifluoromethyl)thiazol-2-yl)-3,3′-bipyridin-6-yl)-3-ethylurea

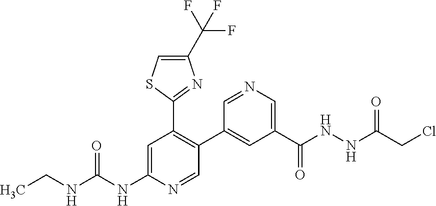

N,N′-methanediylidenedicyclohexanamine (120 mg, 0.58 mmol) was added to a solution of 1-ethyl-3-(5′-(hydrazinecarbonyl)-4-(4-(trifluoromethyl)thiazol-2-yl)-3,3′-bipyridin-6-yl)urea (Intermediate 9, 200 mg, 0.44 mmol), and sodium 2-chloroacetate (51.6 mg, 0.44 mmol) in 1,4-dioxane (10 mL). After stirring at RT for 16 hours the solution was warmed to 50° C. and HATU (200 mg) was added. After stirring for 1 hour, potassium carbonate (100 mg) was added and one hour later pyridine (0.5 ml) was added, and the mixture stirred at 1 hr at 50° C. The solvents were then removed under reduced pressure and the residue was purified by normal phase chromatography on silica gel eluting with a gradient of methanol in dichloromethane to give crude title compound as a pale yellow solid, which was used without further purification.

MS (EI) (M+H)⁺ 528 for C₂₀H₁₈ClF₃N₇O₃S (M−H)⁻ 526 for C₂₀H₁₈ClF₃N₇O₃S;

¹H NMR (DMSO-d6) δ: 10.80 (s, 1H), 10.50 (s, 1H), 9.51 (s, 1H), 9.05 (d, J=1.88 Hz, 1H), 8.66 (d, J=1.88 Hz, 1H), 8.57 (s, 1H), 8.38 (s, 1H), 8.24 (s, 1H), 8.21 (t, J=2.07 Hz, 1H), 7.55 (t, J=5.37 Hz, 1H), 4.21 (s, 2H), 3.05-3.27 (m, 2H), 1.11 (t, J=7.16 Hz, 3H);

¹⁹F NMR (DMSO-d6) δ: −62.43 (s, 3 F)

### Intermediate 465

### 3-bromo-5-(5-oxo-4,5-dihydro-1,3,4-oxadiazol-2-yl)pyridine 1-oxide

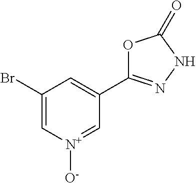

Di(1H-imidazol-1-yl)methanone (295 mg, 1.82 mmol) was added to a solution of 3-bromo-5-(hydrazinecarbonyl)pyridine 1-oxide (Intermediate 466, 310 mg, 1.34 mmol) in DMF (10 mL) and the mixture was stirred for 20 hours. The reaction was then diluted with ethyl acetate (100 ml), water (100 ml), and hydrochloric acid (1N, 10 ml), and the layers were separated. The aqueous phase extracted with ethyl acetate (3×100 ml), and the combined organic layers were washed with brine, dried over magnesium sulfate, and the solvents removed under reduced pressure. The resulting residue was purified by normal phase chromatography on silica gel eluting with a gradient of methanol in dichloromethane to afford 150 mg of the title compound as a pale yellow solid.

MS (EI) (M+H)⁺ 258/2260 for C₇H₅BrN₃O₃ (M−H)⁻ 256/258 for C₇H₃BrN₃O₃;

¹H NMR (DMSO-d6) δ: 13.02 (br. s., 1H), 8.77 (s, 1H), 8.48 (s, 1H), 7.88 (s, 1H);

### Intermediate 466

### 3-bromo-5-(hydrazinecarbonyl)pyridine 1-oxide

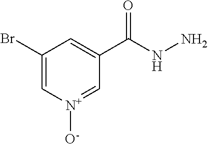

Hydrazine (0.120 g, 3.75 mmol) was added to a solution of 3-bromo-5-(methoxycarbonyl)pyridine 1-oxide (Intermediate 467, 16 g, 3.75 mmol) in ethanol (50 mL), and heated to 70° C. for 19 hours, solvents were removed under reduced pressure and the residue was purified by normal phase chromatography on silica gel eluting with a gradient of methanol in dichloromethane to afford 310 mg of the title compound as a brown gummy solid.

MS (EI) (M+H)⁺ 232/234 for C₆H₇BrN₃O₂ (M−H)⁻ 230/232 for C₆H₅BrN₃O₂;

¹H NMR (DMSO-d6) δ: 10.11 (br. s., 1H), 8.72 (s, 1H), 8.50 (s, 1H), 7.89 (s, 1H), 4.65 (br. s., 2H);

### Intermediate 467

### 3-bromo-5-(methoxycarbonyl)pyridine 1-oxide

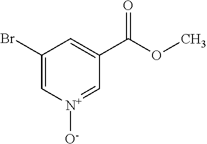

A solution of methyl 5-bromonicotinate (1.6 g, 7.41 mmol) in dichloromethane (25 mL) was treated with 3-chlorobenzoperoxoic acid (1.992 g, 8.89 mmol) and stirred at room temperature for 16 hours. The resulting suspension was filtered, and the filtrate purified by normal phase chromatography on silica gel eluting with a gradient of ethyl acetate in hexane. The major peak was triturated from ethyl acetate with hexanes to give the title compound as a peach solid (1.4 g, 6.03 mmol, 81%).

MS (EI) (M+H)⁺ 232/234 for C₇H₇BrNO₃;

¹H NMR (DMSO-d6) δ: 8.85 (s, 1H), 8.51 (d, J=1.13 Hz, 1H), 7.94 (d, J=1.13 Hz, 1H), 3.89 (s, 3H);

### Intermediate 468

(R)-tert-butyl 1-(5-(6′-(3-ethylureido)-4′-(4-(trifluoromethyl)thiazol-2-yl)-3,3′-bipyridin-5-yl)-1,3,4-oxadiazol-2-yl)-2-methylpropylcarbamate

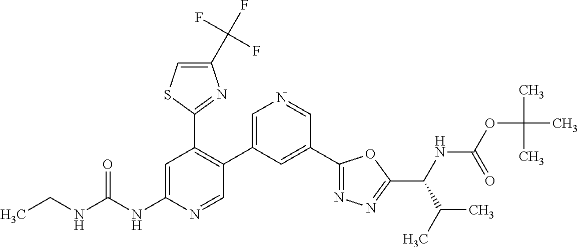

Intermediate 468 was synthesized according to the procedure for Example 264 from Intermediate 461.

MS (EI) (M+H)⁺ 633 for C₂₈H₃₂F₃N₈O₄S (M−H)⁻ 631 for C₂₈H₃₀F₃N₈O₄S;

### Intermediate 469

### (R)-tert-butyl 1-(2-(6′-(3-ethylureido)-4′-(4-(trifluoromethyl)thiazol-2-yl)-3,3′-bipyridine-5-carbonyl)hydrazinyl)-3-methyl-1-oxobutan-2-ylcarbamate

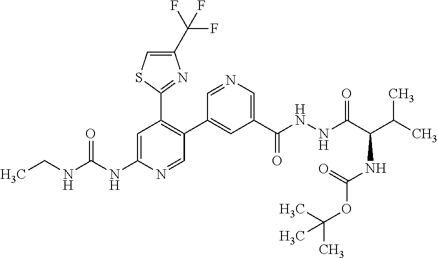

Intermediate 469 was synthesized according to the procedure for Intermediate 268 from Intermediate 9 and (R)-2-(tert-butoxycarbonylamino)-3-methylbutanoic acid.

MS (EI) (M+H)⁺ 651 for C₂₈H₃₄F₃N₈O₅S (M−H)⁻ 649 for C₂₈H₃₂F₃N₈O₅S;

### Intermediate 470

### 1-(4-bromo-5′-(5-oxo-4,5-dihydro-1,3,4-oxadiazol-2-yl)-3,3′-bipyridin-6-yl)-3-ethylurea

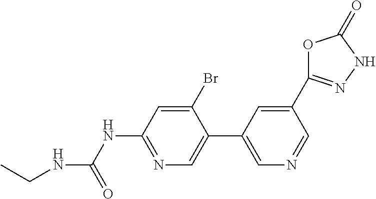

A mixture of (1-(4-bromo-5′-(hydrazine carbonyl)-3,3′-bipyridin-6-yl)-3-ethylurea (Intermediate 471, 60 mg, 0.16 mmol), 1,1′-carbonyldiimmidazol (34.4 mg, 0.21 mmol) and diisopropyl ethylamine (0.041 ml, 0.24 mmol) in DMF (3 ml) was heated at 50° C. for 4 hours, cooled down to room temperature. The crude was concentrated and purified by column chromatography on silica gel (5% methanol in dichloromethane) to give the desired product as a solid (62 mg).

MS (ESP) 407.2 (MH) for C₁₅H₁₃BrN₆O₃.

¹H-NMR (DMSO-d₆) δ: 1.09 (t, 3H); 3.19 (t, 2H); 7.48 (t, 1H); 8.04 (s, 1H); 8.23 (t, 1H); 8.29 (s, 1H); 8.80 (d, 1H); 9.0 (d, 1H); 9.45 (s, 1H).

### Intermediate 471

### 1-(4-bromo-5′-(hydrazinecarbonyl)-3,3′-bipyridin-6-yl)-3-ethylurea-(hydrazinecarbonyl)-3,3′-bipyridin-6-yl)-3-ethylurea

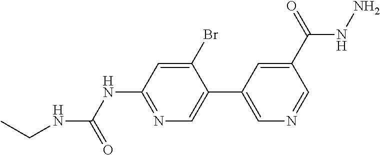

Ethyl 4′-bromo-6′-(3-ethylureido)-3,3′-bipyridine-5-carboxylate (Intermediate 472, 1.32 g, 2.85 mmol), hydrazine hydrate (1.416 ml, 28.53 mmol) were mixed in ethanol (20 ml), heated at 80° C. for 2 d, cooled down to room temperature. The crude was diluted with ethyl acetate; the resulting precipitate was filtered and washed with ethyl acetate, collected as the desired product (920 mg).

MS (ESP) 381.06 (MH⁺) for C₁₄H₁₅BrN₆O₂

¹H-NMR (DMSO-d₆): 1.08 (t, 3H); 3.17 (q, 2H); 3.58 (br, 2H); 7.43 (t, 1H); 8.05 (s, 1H); 8.27 (s, 2H); 8.85 (s, 1H); 9.03 (s, 1H); 9.43 (s, 1H); 11.15 (br, 1H).

### Intermediate 472

### Ethyl 4′-bromo-6′-(3-ethylureido)-3,3′-bipyridine-5-carboxylate

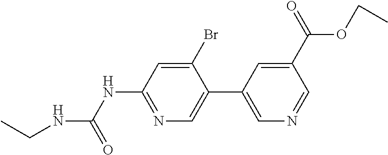

1-(4-Bromo-5-iodopyridin-2-yl)-3-ethylurea (Intermediate 473, 1.33 g, 3.59 mmol), ethyl 5-(4,4,5,5-tetramethyl-1,3,2-dioxaborolan-2-yl)nicotinate (1.049 g, 3.59 mmol), paladium-tetrakistriphenylphosphine (0.415 g, 0.36 mmol) and K₂CO₃ (0.745 g, 5.39 mmol) were suspended in a mixture of DMF (10 ml) and water (1.000 ml). The suspension was degassed and purged with nitrogen then heated at 100° C. for 1.5 h. The reaction mixture was cooled down to room temperature and filtered; the filtrate was concentrated and purified by column chromatography on silica gel to give the desired product (1.32 g).

MS (ESP) 395.02 (MH⁺) for C₁₆H₁₇BrN₄O₃.

¹H-NMR (CDCl₃): 1.29 (t, 3H); 1.45 (t, 3H); 3.45 (q, 2H); 4.47 (q, 2H); 7.30 (br, 1H); 8.12 (s, 1H); 8.38 (t, 1H); 8.84 (2s, 2×H); 9.29 (s, 1H).

### Intermediate 473

### 1-(4-bromo-5-iodopyridin-2-yl)-3-ethylurea

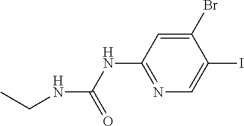

4-bromo-5-iodopyridin-2-amine (Intermediate 474, 3.2 g, 10.71 mmol) was dissolved in dry chloroform (15 mL). Isocyanatoethane (2.52 mL, 32.12 mmol) was added and the reaction mixture was refluxed for 24 hrs. The reaction was cooled down to room temperature and hexanes was added. The resulting precipitate was collected by filtration to give the desired product (3.14 g).

MS (ESP+) 371.99 (MH⁺) for C₈H₉BrIN₃O.

¹H-NMR (DMSO-d₆): 1.06 (t, 3H); 3.32 (q, 2H); 7.24 (br, 1H); 8.05 (s, 1H); 8.52 (s, 1H); 9.31 (s, 1H).

### Intermediate 474

### 4-bromo-5-iodopyridin-2-amine

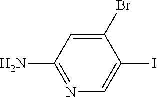

4-Bromopyridin-2-amine (2.5 g, 14.45 mmol) was dissolved in DMF (6 mL)/CHCl₃ (20 mL), 1-iodopyrrolidine-2,5-dione (6.50 g, 28.90 mmol) was added, and the mixture was stirred at 45° C. for 2 days. CHCl₃ was evaporated and the remaining solution was poured into water (15 ml) and extracted with EtOAc (15 ml×3). The organic phase was concentrated and purified by ISCO eluted with Hex/EtOAc (gradient) to give the title compound (3.2 g).

MS (ESP) 298.88 (MH⁺) for C₅H₄BrIN₂.

¹H-NMR (DMSO-d₆): 4.51 (br, 2H); 6.80 (s, 1H); 8.35 (s, 1H).

### Intermediate 475

### 1-ethyl-3-(4-ethynyl-5′-(5-oxo-4,5-dihydro-1,3,4-oxadiazol-2-yl)-3,3′-bipyridin-6-yl)urea

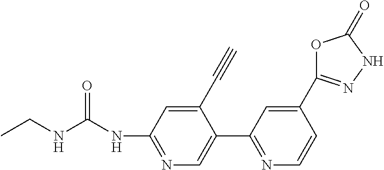

1-ethyl-3-(5′-(5-oxo-4,5-dihydro-1,3,4-oxadiazol-2-yl)-4-((trimethylsilyl)ethynyl)-3,3′-bipyridin-6-yl)urea (Intermediate 476, 84 mg, 0.20 mmol) was suspended in methanol (5 ml), and NaOH (2 ml, 2.00 mmol) was added. The mixture was stirred at room temperature for 2 hrs, aqueous HCl solution (2N) was added to adjust the ph to 6.5. DCM (10 ml) was added and the organic layer was washed with brine and dried over MgSO₄, and concentrated to a volume of 2 ml. Hexanses was added and the resulting precipitate was filtered and washed with DCM, collected as the desired product (25 mg).

MS (ESP) 351 (MH⁺) for C₁₇H₁₄N₆O₃

¹H-NMR (DMSO-d₆): 1.10 (t, 3H); 3.20 (m, 2H); 4.66 (s, 1H); 7.61 (m, 1H); 7.78 (s, 1H); 8.34 (m, 1H); 8.41 (s, 1H); 8.92 (d, 1H); 8.98 (d, 1H); 9.43 (s, 1H); 12.84 (br, 1H) ppm

### Intermediate 476

### 1-ethyl-3-(5′-(5-oxo-4,5-dihydro-1,3,4-oxadiazol-2-yl)-4-((trimethylsilyl)ethynyl)-3,3′-bipyridin-6-yl)urea

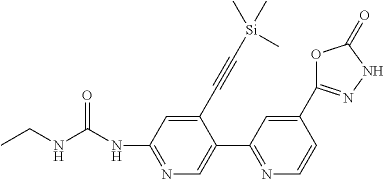

1-(4-bromo-5′-(5-oxo-4,5-dihydro-1,3,4-oxadiazol-2-yl)-3,3′-bipyridin-6-yl)-3-ethylurea (Intermediate 470, 400 mg, 0.99 mmol), ethynyltrimethylsilane (116 mg, 1.18 mmol), copper(I) iodide (18.80 mg, 0.10 mmol), Et₃N (0.550 mL, 3.95 mmol), and Pd(PPh₃)₄ (57.1 mg, 0.05 mmol) were combined in anhydrouse DMF (10 mL) and heated at 80° C. for 4 hours. After cooling down to room temperature, the crude sample was filtered through celite and the filtrate was concentrated and purified by column chromatography on silica gel (Hex/EtOAc) to give the tile compound (160 mg).

MS (ESP) 423 (MH⁺) for C₂₀H₂₂N₆O₃Si

¹H-NMR (DMSO-d₆): 0.12 (s, 9H); 1.10 (t, 3H); 3.20 (m, 2H); 7.57 (m, 1H); 7.72 (s, 1H); 8.41 (m, 1H); 8.45 (s, 1H); 8.92 (d, 1H); 8.99 (d, 1H); 9.41 (s, 1H); 12.86 (s, 1H) ppm

### Intermediate 477

### 1-(4-(azidomethyl)-5′-(5-oxo-4,5-dihydro-1,3,4-oxadiazol-2-yl)-3,3′-bipyridin-6-yl)-3-ethylurea

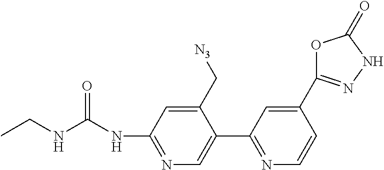

(6-(3-Ethylureido)-5′-(5-oxo-4,5-dihydro-1,3,4-oxadiazol-2-yl)-3,3′-bipyridin-4-yl)methyl methanesulfonate (Intermediate 478, 350 mg, 0.81 mmol), sodium azide (52.4 mg, 0.81 mmol) were mixed in DMF (4 ml), stirred at 60° C. for 2 hr, diluted with dichloromethane, the solvent was evaporated and the residue was mixed with silica gel and dry loaded onto isco column (silica gel), eluted with 10% MeOH in dichloromethane to give the title product as a white solid (206 mg).

MS (ESP) 382 (MH⁺) for C₁₆H₁₅N₉O₃

¹H-NMR (DMSO-d₆): 1.10 (t, 3H); 3.20 (m, 2H); 4.55 (s, 2H); 7.69 (s, 1H); 7.81 (t, 1H); 8.18 (m, 2H); 8.77 (d, 1H); 9.00 (d, 1H); 9.40 (s, 1H); 12.84 (s, 1H) ppm

### Intermediate 478

### (6-(3-ethylureido)-5′-(5-oxo-4,5-dihydro-1,3,4-oxadiazol-2-yl)-3,3′-bipyridin-4-yl)methyl methanesulfonate

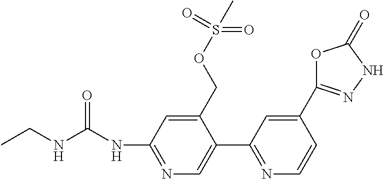

1-Ethyl-3-(4-(hydroxymethyl)-5′-(5-oxo-4,5-dihydro-1,3,4-oxadiazol-2-yl)-3,3′-bipyridin-6-yl)urea (Intermediate 479, 420 mg, 1.18 mmol), methane sulfonyl chloride (0.137 ml, 1.77 mmol) were mixed in DMF (4 ml)/DCM (15 ml) and stirred at 25° C. for 2 hr. The reaction mixture was then diluted with DCM (10 ml), washed with brine and the organic phase was concentrated and diluted in dichloromethane. Hexanses was added and the resulting precipitate was filtered and collected as the desired product (350 mg).

MS (ESP) 435 (MH⁺) for C₁₇H₁₈N₆O₆S

### Intermediate 479

### 1-Ethyl-3-(4-(hydroxymethyl)-5′-(5-oxo-4,5-dihydro-1,3,4-oxadiazol-2-yl)-3,3′-bipyridin-6-yl)urea

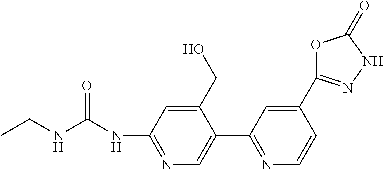

1-Ethyl-3-(5′-(hydrazine carbonyl)-4-(hydroxymethyl)-3,3′-bipyridin-6-yl)urea (Intermediate 480, 470 mg, 1.42 mmol), CDI (476 mg, 2.85 mmol) and DIEA (0.497 ml, 2.85 mmol) were suspended in DMF (5 ml) and stirred at room temperature for 12 hours. The reaction mixture turned into solution, and sodium hydroxide (aqueous solution, 2N, 1 ml) was added, and the reaction was stirred for 30 min. Hydrogen chloride (aqueous, 2N, ˜1 ml) was added, and the mixture was extracted with 10% methanol in dichloromethane (10 ml×5). The organic layer was dried over anhydrous MgSO₄, filtered and concentrated to volume of ˜3 ml, ether (10 ml) was added, the resulting precipitates was filtered and washed with ether and DCM, collected to give the title compound (425 mg).

MS (ESP) 357 (MH⁺) for C₁₆H₁₆N₆O₄

¹H-NMR (DMSO-d₆): 1.10 (t, 3H); 3.19 (m, 2H); 4.41 (s, 2H); 5.47 (m, 1H); 7.65 (s, 1H); 8.10 (s, 1H); 8.18 (m, 2H); 8.70 (br, 1H); 8.95 (d, 1H); 9.36 (s, 1H); 12.84 (s, 1H) ppm

### Intermediate 480

### 1-ethyl-3-(5′-(hydrazine carbonyl)-4-(hydroxymethyl)-3,3′-bipyridin-6-yl)urea

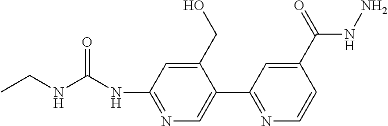

Ethyl 6′-(3-ethylureido)-4′-(hydroxymethyl)-3,3′-bipyridine-5-carboxylate (Intermediate 481, 500 mg, 1.45 mmol), hydrazine hydrate (0.721 ml, 14.52 mmol) were mixed in ethanol (10 ml), heated at 80° C. for 25 hr., cooled down to room temperature, ethyl acetate was added and the resulting solid was filtered and washed with ethyl acetate, to give the desired product (440 mg).

MS (ESP) 331 (MH⁺) for C₁₅H₁₈N₆O₃

### Intermediate 481

### Ethyl 6′-(3-ethylureido)-4′-(hydroxymethyl)-3,3′-bipyridine-5-carboxylate

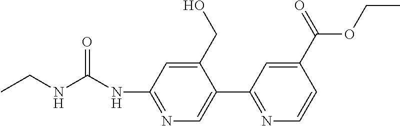

In a round bottom flask, 1-(5-bromo-4-(hydroxymethyl)pyridin-2-yl)-3-ethylurea (Intermediate 482, 5.26 g, 19.19 mmol), ethyl 5-(4,4,5,5-tetramethyl-1,3,2-dioxaborolan-2-yl)nicotinate (5.60 g, 19.19 mmol), Pd(PPh₃)₄ (2.217 g, 1.92 mmol) and cesium carbonate (12.50 g, 38.38 mmol) were suspended in a 4:1 mixture of 1,4 dioxane/water. The suspension was degassed and purged with nitrogen then heated in the microwave at 100° C. for 2 hours. The reaction was cooled to room temperature. Diluted with DCM (20 ml)/MeOH (5 ml) and washed with brine. The organic phase was dried and concentrated then purified by column chromatography on silica gel eluted with DCM/MeOH (95/5%) to give the title compound as a solid. (3.5 g)

MS (ESP) 345 (MH⁺) for C₁₇H₂₀N₄O₄

¹H-NMR (DMSO-d₆): 1.27 (t, 3H); 1.45 (t, 3H); 3.43 (m, 2H); 4.46 (q, 2H); 4.65 (s, 2H); 8.02 (br, 1H); 8.30 (br, 2H); 8.77 (s, 1H); 9.28 (s, 1H).

### Intermediate 482

### 1-(5-bromo-4-(hydroxymethyl)pyridin-2-yl)-3-ethylurea

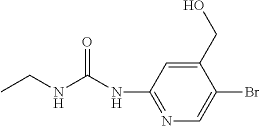

Methyl 5-bromo-2-(3-ethylureido) isonicotinate (Intermediate 483, 5 g, 15.82 mmol), NaBH₄ (1.795 g, 47.45 mmol) were mixed in EtOH (20 ml), and the mixture was refluxed for over night. The solvent was evaporated and the residue was mixed with DCM (50 ml). A 2N HCl aqueous solution was added (10 ml), and the mixture was stirred for 10 min, then the saturated NaHCO₃ was added and the mixture was extracted with DCM (20 ml×3), the organic layer was dried over MgSO₄, filtered and the volume was reduced to 10 ml. The precipitate that formed was filtered and collected as the desired product (2.34 g).

MS (ESP) 275 (MH⁺) for C₉H₁₂BrN₃O₂

¹H-NMR (CD₃OD): 1.20 (t, J=7.33 Hz, 3H); 4.59 (s, 2H); 7.35 (s, 1H); 8.22 (s, 1H) ppm.

### Intermediate 483

### Methyl 5-bromo-2-(3-ethylureido) isonicotinate

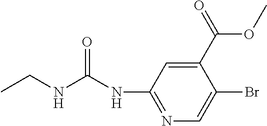

To a solution of methyl 2-amino-5-bromoisonicotinate (25 g, 108.20 mmol) in chloroform (20 mL) was added ethyl isocyanate (17.00 mL, 216.41 mmol), and the reaction mixture heated to reflux for 16 h then cooled to ambient temperature. The product was precipitated with hexane (200 mL), filtered, washed with hexane (2×50 mL) and dried to give 27.5 g light yellow color solid as the desired product.

MS (ESP): 304.00 (M+2) for C₁₀H₁₂BrN₃O₃

¹H NMR (DMSO-d₆): 1.07 (t, J=7.20 Hz, 3H); 3.01-3.25 (m, 2H); 3.91 (s, 3H); 7.17 (t, J=5.31 Hz, 1H); 8.02 (d, J=1.52 Hz, 1H); 8.46 (s, 1H); 9.41 (s, 1H)

### Intermediate 484

### 2-(5-bromo-6-(tetrahydro-2H-pyridin-3-yl)-5-methyl-1,3,4-oxadiazole

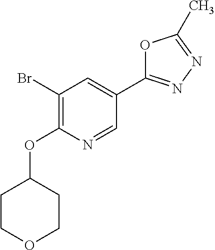

A solution of tetrahydro-2H-pyran-4-yl 5-bromo-6-(tetrahydro-2H-pyran-4-yloxy)nicotinate (Intermediate 281, 2 g) and hydrazine (2 mL) in ethanol (20 mL) was heated to reflux for 20 h. The reaction mixture was cooled to room temperature and concentrated under reduced pressure. The crude product and 1,1,1-trimethoxyethane (20 ml, 166.46 mmol) was heated reflux and treated with concentrated aqueous HCl (drop), the resulting clear colorless solution was refluxed for 20 minutes, treated with DBU (0.2 ml, 1.33 mmol) and refluxed an additional 20 min. The material was concentrated under reduced pressure to give a tan gum which was purified by flash chromatography on silica gel eluting with a gradient of ethyl acetate in hexanes to give the title compound as a white solid (1.77 g)

MS (EI) (M+H)⁺ 340/342 for C₁₃H₁₄BrN₃O₃;

### Intermediate 485

### 3-bromo-5-(5-oxo-4,5-dihydro-1,3,4-oxadiazol-2-yl)pyridine

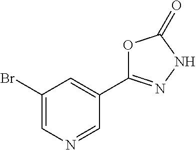

Di(1H-imidazol-1-yl)methanone (290 mg, 1.82 mmol) was added to a solution of 3-bromo-5-(hydrazinecarbonyl)pyridine (Intermediate 433, 300 mg, 1.34 mmol) in DMF (10 mL) and the reaction was allowed to proceed for 20 hours. The reaction was then diluted with ethyl acetate (100 ml), water (100 ml), and hydrochloric acid (1N, 10 ml), and the layers were separated. The aqueous phase extracted with ethyl acetate (3×100 ml), and the combined organics were washed with brine, dried over magnesium sulfate, filtered and the solvents were removed under reduced pressure. The resulting residue was purified by normal phase chromatography on silica gel eluting with a gradient of methanol in dichloromethane to afford 150 mg of the title compound as a pale yellow solid.

MS (EI) (M+H)⁺ 242/250 for C₇H₅BrN₃O₂;

### Intermediate 486

### 6-(6-(3-ethylureido)-4-(4-(6-methoxypyridin-2-yl)thiazol-2-yl)pyridin-3-yl)pyrazine-2-carboxylic acid

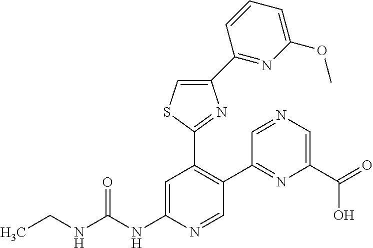

A mixture of 6-(3-ethylureido)-4-(4-(6-methoxypyridin-2-yl)thiazol-2-yl)pyridin-3-ylboronic acid (Intermediate 415, 700 mg, 0.88 mmol), 6-chloropyrazine-2-carboxylic acid (278 mg, 1.75 mmol), cesium carbonate (571 mg, 1.75 mmol), dipalladium tridibenzilidine acetone (40.1 mg, 0.04 mmol), Dicyclohexyl TriisopropylBiphenyl phosphine (84 mg, 0.18 mmol), in 1,4-dioxane (12 mL) and water (3.0 mL) was degassed, then heated in a microwave reactor at 110° C. for 1 hr. The reaction was diluted with ethyl acetate (25 ml), and the resulting solid was removed by filtration, the solid was washed with ethyl acetate, ethyl acetate/methanol, and 1N sodium hydroxide. The combined filtrates were extracted with 1N sodium hydroxide (3×50 ml). The combined aqueous extracts were acidified (con. HCl), and the aqueous phase was extracted with ethyl acetate (3×50 ml). The combined organics were washed with brine, dried over magnesium sulfate, filtered, and the solvent was removed under reduced pressure. The resulting solid was washed exhaustively with methanol solutions in methylene chloride to afford 30 mg of the title compound as a beige solid.

MS (EI) (M+H)⁺ 478 for C₂₅H₂₀N₇O₄S (M−H)⁻ 476 for C₂₅H₁₈N₇O₄S;

¹H NMR (DMSO-d6) δ: 9.62 (s, 1H), 9.13 (s, 1H), 8.85 (s, 1H), 8.49 (s, 1H), 8.36 (s, 1H), 8.28 (s, 1H), 7.70 (t, J=7.82 Hz, 1H), 7.63 (br. s., 1H), 6.96 (d, J=7.35 Hz, 1H), 6.76 (d, J=8.29 Hz, 1H), 3.91 (s, 3H), 3.13-3.28 (m, 2H), 1.12 (t, J=7.25 Hz, 3H)

1. A compound of formula (I): 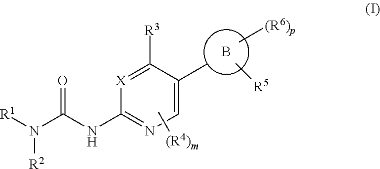
or a pharmaceutically acceptable salt thereof, wherein:
X is N, CH or CR⁴;
R¹ is selected from C₁₋₆alkyl, C₂₋₆alkenyl, C₂₋₆alkynyl or C₃₋₆cycloalkyl; wherein R¹ may be optionally substituted on carbon by one or more R⁷;
R² is selected from hydrogen or C₁₋₆alkyl; wherein said C₁₋₆alkyl may be optionally substituted by one or more groups independently selected from halo, cyano, hydroxy, nitro and amino;
or R¹ and R² together with the nitrogen to which they are attached form a heterocyclyl; wherein said heterocyclyl may be optionally substituted on one or more carbon atoms with one or more R⁸; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹;
R³ is a C₃₋₁₄carbocyclyl or a heterocyclyl; wherein the carbocyclyl or heterocyclyl may be optionally substituted on one or more carbon atoms by one or more R¹⁰; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹¹;
R⁴, for each occurrence, is independently selected from the group consisting of halo, nitro, cyano, hydroxy, amino, mercapto, C₁₋₆alkyl, C₂₋₆alkenyl, C₂₋₆alkynyl, C₁₋₆alkoxy, N—(C₁₋₆alkyl)amino, N,N—(C₁₋₆alkyl)₂amino, and C₁₋₆alkylsulfanyl; wherein R⁴, for each occurrence, is independently optionally substituted on one or more carbon atoms with one or more R¹²;
R⁵ is hydrogen or a heterocyclyl; wherein the heterocyclyl may be optionally substituted on one or more carbon atoms with an ═O, ═S, or one or more R¹⁴; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹⁵;
R⁶, for each occurrence, is independently selected from the group consisting of halo, nitro, cyano, hydroxy, amino, mercapto, sulphamoyl, ═O, ═S, C₁₋₆alkyl, C₂₋₆alkenyl, C₂₋₆alkynyl, C₁₋₆alkoxy, N—(C₁₋₆alkyl)amino, N,N—(C₁₋₆alkyl)₂amino, C₁₋₆alkylS(O)ₐ— wherein a is 0, 1 or 2, N—(C₁₋₆alkyl)sulphamoyl, N,N—(C₁₋₆alkyl)₂sulphamoyl, C₁₋₆alkylsulphonylamino, N′-hydroxycarbamimidoyl, carbamimidoyl, C₃₋₁₄carbocyclyl-L- and heterocyclyl-L-; wherein R⁶, for each occurrence, is independently optionally substituted on one or more carbon atoms with one or more R¹⁶; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹³;
m is 0 or 1;
p is 0, 1, 2, or 3;
Ring B is C₃₋₁₄carbocyclyl or heterocyclyl; wherein if said heterocyclyl contains an —NH— moiety that nitrogen may be optionally substituted by a group selected from R¹⁵; and wherein if said heterocyclyl contains an ═N— or a —S— moiety that nitrogen may be optionally substituted by one oxo group and that sulfur may be optionally substituted by one or two oxo groups;
R⁷, R⁸, R¹⁰, R¹², R¹⁴ and R¹⁶ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, C₃₋₆ carbocyclyl-L- or heterocyclyl-L-; wherein R⁷, R⁸, R¹⁰, R¹², R¹⁴ and R¹⁶ independently of each other may be optionally substituted on one or more carbon by one or more R¹⁹; and wherein if said heterocyclyl contains an —NH— moiety that nitrogen may be optionally substituted by a group selected from R²⁰; and wherein if said heterocyclyl contains an ═N— or a —S— moiety that nitrogen may be optionally substituted by one oxo group and that sulfur may be optionally substituted by one or two oxo groups
R⁹, R¹¹, R¹³, R¹⁵, and R²⁰, for each occurrence, are independently selected from C₁₋₆alkyl, C₃₋₆cycloalkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, imidazolylcarbonyl, amino, benzoyl and phenylsulphonyl; wherein R⁹, R¹¹, R¹³, R¹⁵, and R²⁰ independently of each other may be optionally substituted on carbon by one or more R²³;
R¹⁹ and R²³, for each occurrence, are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, 2-methoxyethoxy, morpholinyl, piperazinyl, acetyl, acetoxy, methylamino, ethylamino, dimethylamino, diethylamino, N-methyl-N-ethylamino, N-(2-morpholinoethyl)-amino, cyclohexylamino, cyclopentylamino, cyclohexyl, acetylamino, 2-methyoxyethylamino, tetrahydropyran-4-ylamino, N-methylcarbamoyl, N-ethylcarbamoyl, N,N-dimethylcarbamoyl, N,N-diethylcarbamoyl, N-methyl-N-ethylcarbamoyl, benzyloxy, 9H-fluoren-9-ylmethoxycarbonylamino, t-butoxycarbonylamino, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; and
L is a direct bond, —O—, —C(O)—, —C(O)NR²⁵—, —NR²⁵C(O)—, or —CH₂—; and
R²⁵ is H or a C₁₋₆alkyl.

2. The compound of claim 1, or pharmaceutically acceptable salts thereof, wherein X is CH.

3. The compound of claim 1, or pharmaceutically acceptable salts thereof, wherein X is N.

4. The compound of claim 2, or pharmaceutically acceptable salts thereof, wherein R¹ is a C₁₋₆alkyl.

5. The compound of claim 4, or pharmaceutically acceptable salts thereof, wherein R¹ is ethyl.

6. The compound of claim 4, or pharmaceutically acceptable salts thereof, wherein R² is hydrogen.

7. The compound of claim 6, or pharmaceutically acceptable salts thereof, wherein m is 0.

8. The compound of claim 2, or pharmaceutically acceptable salts thereof, wherein ring B is a 5- or 6-membered heteroaryl, wherein if said heteroaryl contains an —NH— moiety that nitrogen may be optionally substituted by a group selected from R¹⁵; and wherein if said heteroaryl contains an ═N— or a —S— moiety that nitrogen may be optionally substituted by one oxo group and that sulfur may be optionally substituted by one or two oxo groups.

9. The compound of claim 8, or pharmaceutically acceptable salts thereof, wherein ring B is pyridinyl, pyrazinyl, pyrimidinyl or thiazolyl, wherein each ═N— of pyridinyl, pyrazinyl, pyrimidinyl, or thiazolyl may be independently optionally substituted with one oxo group; and wherein the —S— moiety of the thiazolyl may be optionally by one or two oxo groups.

10. The compound of claim 2, or pharmaceutically acceptable salts thereof, wherein ring B is a bicyclic heterocyclyl, wherein if said heterocyclyl contains an —NH— moiety that nitrogen may be optionally substituted by a group selected from R¹⁵; and wherein if said heterocyclyl contains an ═N— or a —S— moiety that nitrogen may be optionally substituted by one oxo group and that sulfur may be optionally substituted by one or two oxo groups.

11. The compound of claim 10, or pharmaceutically acceptable salts thereof, wherein ring B is a quinoxalinyl, 5,6-dihydro[1,3]thiazolo[4,5-d]pyridazine-4,7-dione or 2,3-dihydrophthalazine-1,4-dione; and wherein each —NH— moiety of 5,6-dihydro[1,3]thiazolo[4,5-d]pyridazine-4,7-dione or 2,3-dihydrophthalazine-1,4-dione may be independently optionally substituted by a group selected from R¹⁵; and wherein each ═N— of quinoxalinyl or 5,6-dihydro[1,3]thiazolo[4,5-d]pyridazine-4,7-dione may be independently optionally substituted with one oxo group; and wherein the —S— moiety of the 5,6-dihydro[1,3]thiazolo[4,5-d]pyridazine-4,7-dione may be optionally by one or two oxo groups.

12. The compound of claim 8, or pharmaceutically acceptable salts thereof, wherein R³ is a 5-membered heteroaryl; wherein the heteroaryl may be optionally substituted on one or more carbon atoms by one or more R¹⁰; and wherein if said heteroaryl contains an ═N— or a —S— moiety that nitrogen may be optionally substituted by one oxo group and that sulfur may be optionally substituted by one or two oxo groups; and wherein if said heteroaryl contains an —NH— moiety that nitrogen may be optionally substituted by a group selected from R¹¹.

13. The compound of claim 12, or pharmaceutically acceptable salts thereof, wherein R³ is a thiazolyl; wherein the thiazolyl may be optionally substituted on carbon by one or more R¹⁰; and wherein the ═N— of the thiazolyl may be optionally substituted by one oxo group; and wherein the —S— of the thiazolyl may be optionally substituted by one or two oxo groups.

14. The compound of claim 12, or pharmaceutically acceptable salts thereof, wherein R³ is a 1,3,4-oxadiazolyl; wherein the 1,3,4-oxadiazolyl may be optionally substituted on one or more carbon by one or more R¹⁰; and wherein each ═N— of the 1,3,4-oxadiazolyl may be independently optionally substituted by one oxo group.

15. The compound of claim 12, or pharmaceutically acceptable salts thereof, wherein R³ is a 1H-pyrazolyl; wherein the 1H-pyrazolyl may be optionally substituted on one or more carbon by one or more R¹⁰; and wherein the ═N— of the 1H-pyrazolyl may be optionally substituted by one oxo group; and wherein the —NH— moiety of the 1H-pyrazolyl may be optionally substituted by a group selected from R¹¹.

16. The compound of claim 8, or pharmaceutically acceptable salts thereof, wherein R³ is 1,3-benzothiazolyl; wherein the 1,3-benzothiazolyl may be optionally substituted on one or more carbon by one or more R¹⁰; and wherein the ═N— of the 1,3-benzothiazolyl may be optionally substituted by one oxo group; and wherein the —S— of the 1,3-benzothiazolyl may be optionally substituted by one or two oxo groups.

17. The compound of claim 12, or pharmaceutically acceptable salts thereof, wherein R¹⁰ is selected from the group consisting of methyl, phenyl, trifluoromethyl, and pyridinyl.

18. The compound of claim 11, or pharmaceutically acceptable salts thereof, wherein R¹¹ is methyl.

19. The compound of claim 9, or pharmaceutically acceptable salts thereof, wherein, R³ is 4-trifluoromethyl-thiazol-2-yl, 4-(pyridin-2-yl)-thiazol-2-yl, 4-phenyl-thiazol-2-yl, 1,3-benzothiazol-2-yl, 2-(pyridin-4-yl)-1,3,4-oxadiazol-5-yl, 1-methyl-1H-pyrazol-5-yl, 1-methyl-1H-pyrazol-4-yl, 2-methyl-1,3,4-oxadiazol-5-yl, or 4-(pyridin-4-yl)-thiazol-2-yl.

20. The compound of claim 12, or pharmaceutically acceptable salts thereof, wherein R⁵ is a five membered aromatic heterocyclyl; wherein the heterocyclyl may be optionally substituted on one or more carbon atoms with one or more R¹⁴; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹⁵.

21. The compound of claim 20, or pharmaceutically acceptable salts thereof, wherein R⁵ is selected from the group consisting of 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, morpholinyl, 4,5-dihydro-oxazolyl, and 1H-1,2,4-triazolyl, wherein the 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, morpholinyl, 4,5-dihydro-oxazolyl, and 1H-1,2,4-triazolyl may be optionally substituted on one or more carbon atoms with one or more R¹⁴; and wherein the ═N— moiety of 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, 4,5-dihydro-oxazolyl, and 1H-1,2,4-triazolyl may be optionally substituted with one oxo group and the —S— moiety of 1,3,4-thiadiazolyl or the 3H-1,2,3,5-oxathiadiazolyl, may be optionally substituted by one or two oxo groups; and wherein the —NH— moiety of the 1H-tetrazolyl, 1H-pyrazolyl, 3H-1,2,3,5-oxathiadiazolyl, 1H-imidazolyl, morpholinyl, or the 1H-1,2,4-triazolyl may be optionally substituted by a group selected from R¹⁵.

22. The compound of claim 20, or pharmaceutically acceptable salts thereof, wherein R⁵ is 5-oxo-4,5-dihydro-1,3,4-oxadiazo-2-yl.

23. The compound of claim 20, or pharmaceutically acceptable salts thereof, wherein R¹⁴ is selected from the group consisting of C₁₋₄alkyl or hydroxy.

24. The compound of claim 20, or pharmaceutically acceptable salts thereof, wherein R¹⁵ is a C₁₋₄alkyl.

25. The compound of claim 20, or pharmaceutically acceptable salts thereof, wherein p is 0.

26. The compound of claim 20, or pharmaceutically acceptable salts thereof, wherein p is 1.

27. The compound of claim 26, or pharmaceutically acceptable salts thereof, wherein R⁶ is cyano, bromo, methylsulfonyl, sulphamoyl, or butyloxy.

28. The compound of claim 12, or pharmaceutically acceptable salts thereof, wherein R⁵ is hydrogen.

29. The compound of claim 28, or pharmaceutically acceptable salts thereof, wherein p is 0.

30. The compound of claim 29, or pharmaceutically acceptable salts thereof, wherein ring B is pyridine or quinoxalinyl

31. The compound of claim 28, or pharmaceutically acceptable salts thereof, wherein p is 1 and R⁶ is cyano, bromo, methylsulfonyl, or sulphamoyl.

32. A pharmaceutical composition comprising a compound of claim 1, or a pharmaceutically acceptable salt thereof, and a pharmaceutically acceptable excipient or carrier.

33. A method of inhibiting bacterial DNA gyrase and/or bacterial topoisomerase IV in a warm-blooded animal in need of such treatment, comprising administering to the animal an effective amount of a compound of claim 1, or a pharmaceutically acceptable salt thereof.

34. A method of producing an antibacterial effect in a warm-blooded animal in need of such treatment, comprising administering to the animal an effective amount of a compound of claim 1, or a pharmaceutically acceptable salt thereof.

35. A method of treating a bacterial infection in a warm-blooded animal in need thereof, comprising administering to the animal an effective amount of a compound of claim 1, or a pharmaceutically acceptable salt thereof.

36. The method of claim 35, wherein the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

37. The method of claim 35, wherein the warm-blooded animal is a human.
